(19) United States
(12) Patent Application Publication
(10) Pub. No.: US 2008/0051326 A1
(43) Pub. Date: Feb. 28, 2008

(54) Antiinfective Lipopeptides

(76) Inventors: Dylan Christopher Alexander, Watertown, MA (US); Richard H. Baltz, Lincoln, MA (US); Paul Brian, Maynard, MA (US); Marie-Francoise Coeffet-Le Gal, Arlington, MA (US); Sascha Doekel, Cambridge, MA (US); Xiaowei He, Lexington, MA (US); Vidya Kulkarni, Waltham, MA (US); Christopher Leitheiser, Burlington, MA (US); Vivian Pak Woon Miao, Surrey (CA); Kien Trung Nguyen, Arlington, MA (US); Ian Barrie Parr, Medford, MA (US); Daniel Ritz, Luzern (CH); Yanzhi Zhang, Sharon, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/667,645

(22) PCT Filed: Nov. 11, 2005
(86) PCT No.: PCT/US05/40919

§ 371 Date: May 10, 2007

Related U.S. Application Data
(60) Provisional application No. 60/627,056, filed on Nov. 12, 2004.
(60) Provisional application No. 60/710,705, filed on Aug. 23, 2005.

(51) Int. Cl. A61K 38/12; A61P 31/04; C07K 7/64

(52) U.S. Cl. 514/11; 530/317

(57) ABSTRACT

The present invention relates to novel depsipeptide compounds. The invention also relates to pharmaceutical compositions of these compounds and methods of using these compounds as antibacterial compounds. The invention also relates to methods of producing these novel depsipeptide compounds and intermediates used in producing these compounds.

### CROSS-REFERENCE TO RELATED APPLICATIONS

The present application claims the benefit of U.S. Provisional Application Nos. 60/710,705, filed Aug. 23, 2005 and 60/627,056, filed Nov. 12, 2004, which are hereby incorporated by reference in their entirety.

### GOVERNMENT SUPPORT

Portions of the work described herein were made with government support under Small Business Innovation Research (SBIR) Grant No. 5R44GM068173-03 and Grant No. 1R43A156858-1. The government may have certain rights to such work.

### FIELD OF THE INVENTION

The present invention relates to novel depsipeptides compounds. The invention also relates to pharmaceutical compositions of these compounds and methods of using these compounds as antibacterial agents.

### BACKGROUND OF THE INVENTION

The rapid increase in the incidence of gram-positive infections—including those caused by resistant bacteria—has sparked renewed interest in the development of novel classes of antibiotics. A class of compounds that has shown potential as useful antibiotic agents is the cyclic depsipeptides. A notable member of the cyclic depsipeptides is the A21978C lipopeptides described in, for example, U.S. Pat. Nos. RE 32,333; RE 32,455; RE 32,311; RE 32,310; 4,482,487; 4,537,717; 5,912,226; 6,911,525; and 6,794,490 and International Patent Applications WO01/44272; WO01/44274; and WO01/44271. Additionally, the A54145 class of compounds described in U.S. Pat. Nos. 4,994,270; 5,039,789; and 5,028,590 have also been shown to possess antibiotic activity.

Daptomycin, also known as LY146032, is comprised of an n-decanoyl side chain linked to the N-terminal tryptophan of a three-amino acid chain linked to a cyclic 10-amino acid peptide. Daptomycin has potent bactericidal activity in vitro and in vivo against clinically relevant gram-positive bacteria that cause serious and life-threatening diseases. These bacteria include resistant pathogens, such as vancomycin-resistant enterococci (VRE), methicillin-resistant Staphylococcus aureus (MRSA), glycopeptide intermediate susceptible Staphylococcus aureus (GISA), vancomycin-resistant Staphylococcus aureus (VRSA), coagulase-negative staphylococci (CNS), and penicillin-resistant Streptococcus pneumoniae (PRSP), for which there are few therapeutic alternatives. See, e.g., Tally et al., 1999, Exp. Opin. Invest. Drugs 8:1223-1238.

Despite the promise that existing antibacterial agents have shown, the need for novel antibiotics continues. Many pathogens have been repeatedly exposed to commonly used antibiotics. This exposure has led to the selection of variant antibacterial strains resistant to a broad spectrum of antibiotics. The loss of potency and effectiveness of an antibiotic caused by resistant mechanisms renders the antibiotic ineffective and consequently can lead to some life-threatening infections that are virtually untreatable. As new antibiotics come to market, pathogens may develop resistance or intermediate resistance to these new drugs, effectively creating a need for a stream of new antibacterial agents to combat these emerging strains. In addition compounds that exhibit bactericidal activity offer advantages over present bacteriostatic compounds. Thus, novel antibacterial agents would be expected to be useful to treat not only “natural” pathogens, but also intermediate drug resistant and drug resistant pathogens because the pathogen has never been exposed to the novel antibacterial agent. New antibacterial agents may exhibit differential effectiveness against different types of pathogens.

### SUMMARY OF THE INVENTION

The present invention provides novel compounds that have antibacterial activity against a broad spectrum of bacteria, including drug-resistant bacteria, and processes for making these compounds.

The present invention provides, in one aspect, compounds of Formula I: 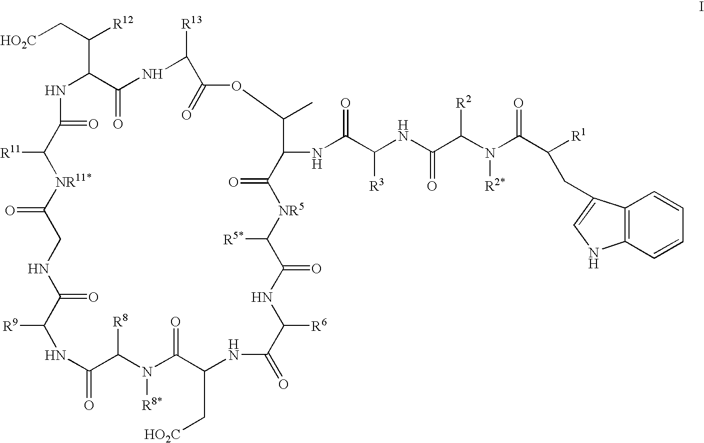
and salts thereof; wherein:

a) R² is an amino acid side chain,

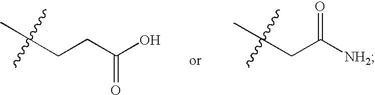

b) R²* is H or alternatively R² together with R²* forms a five or six-member heterocyclic ring;

c) R³ is 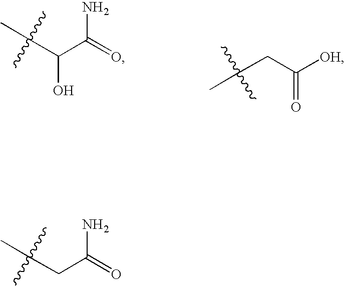
or a non-proteinogenic amino acid side chain;

d) R⁵ is H or methyl;

e) R⁵* is H or an amino acid side chain derived from an N-methylamino acid.

Alternatively R⁵ together with R⁵* forms a five or six-member heterocyclic ring;

f) R⁶ is methyl or

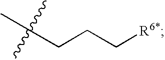

g) R⁸ is an amino acid side chain, methyl,

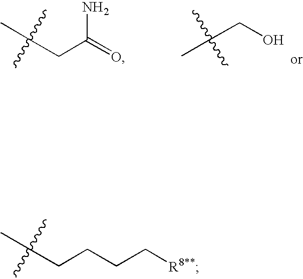

h) R⁸* is H or, alternatively, R⁸ together with R⁸* forms a five or six-member heterocyclic ring;

i) R⁹is 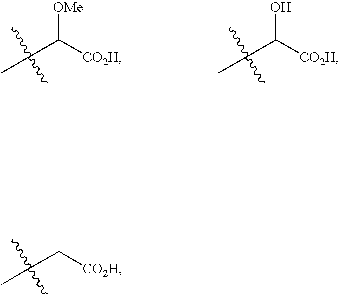
or an amino acid side chain substituted with at least one carboxylic acid;

j) R¹¹ is an amino acid side chain, methyl,

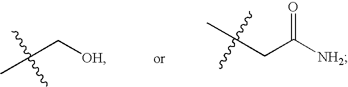

k) R¹¹* is H or, alternatively, R¹¹ together with R¹¹* forms a five or six-member heterocyclic ring;

l) R¹² is H or CH₃

m) R¹³ is CH(CH₃)₂, CH(CH₂CH₃)CH₃, 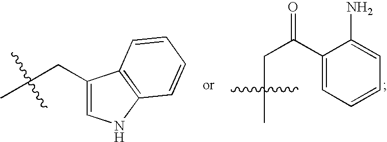
and

n) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides a compound of the Formula F1: 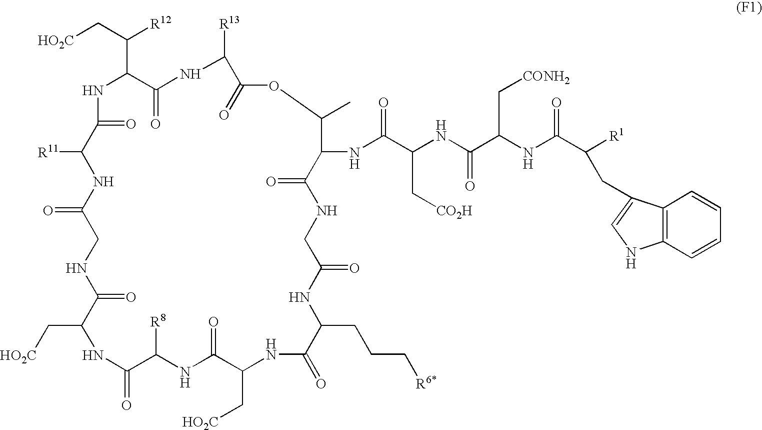
and salts thereof, wherein:
a) R⁸ is hydrogen, 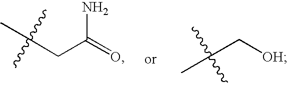
b) R¹¹ is methyl, 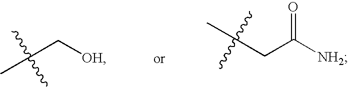
c) R¹² is H or CH₃;
d) R¹³ is CH(CH₃)₂, CH(CH₂CH₃)CH₃, 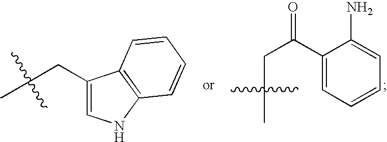
   and
e) each of R¹ and R⁶* is independently amino, monosubstituted amino, disubstituted amino, NH-amino protecting group, acylamino, ureido, guanidino, carbamoyl, sulfonamino, thioacylamino, thioureido, iminoamino, or phosphonamino;

The present invention provides, in another aspect, compounds of Formula F2: 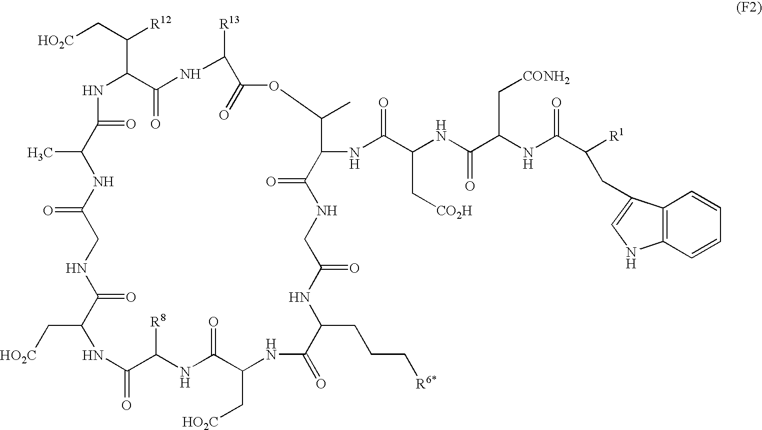
and salts thereof; wherein:
a) R⁸ is hydrogen, methyl, 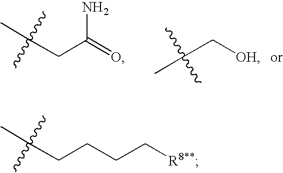
b) R¹² is H or CH₃,
c) R¹³ is CH(CH₃)₂, CH(CH₂CH₃)CH₃, 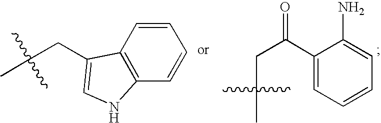
   and
d) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino;

In another aspect, the invention provides compounds of Formula F3: 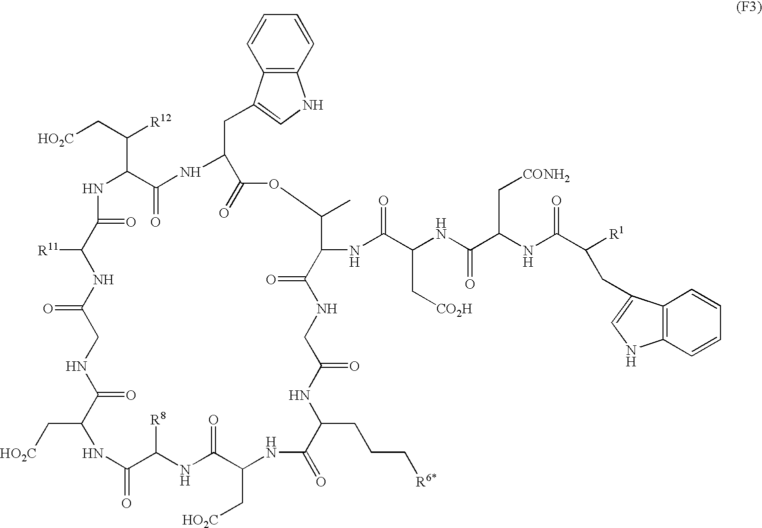
and salts thereof, wherein:
a) R⁸ is hydrogen, 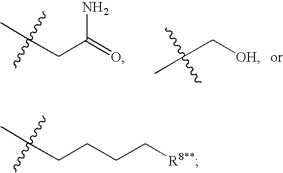
b) R¹¹ is methyl, 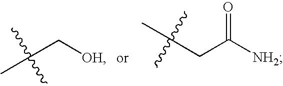
c) R¹² is H or CH₃; and
d) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

The present invention provides, in another aspect, compounds of Formula F4: 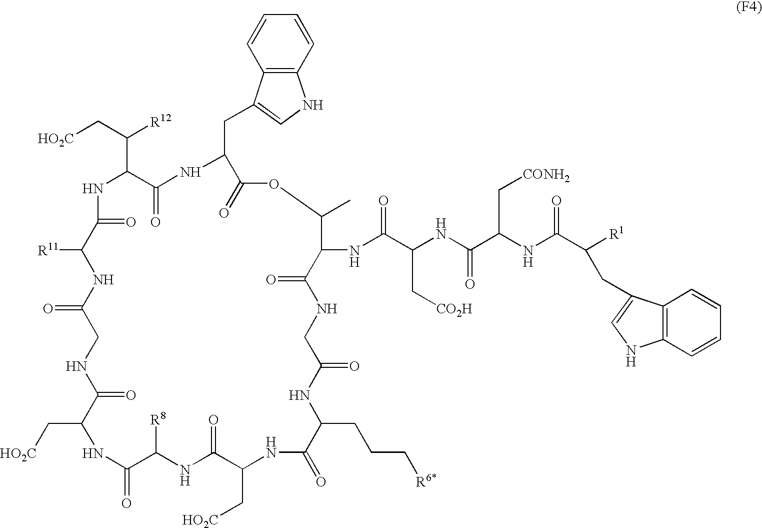
and salts thereof, wherein:
a) R⁸ is hydrogen, methyl, 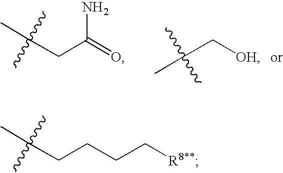
b) R¹¹ is methyl, or 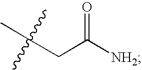
c) R¹² is H or CH₃; and
d) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides compounds of Formula F5: 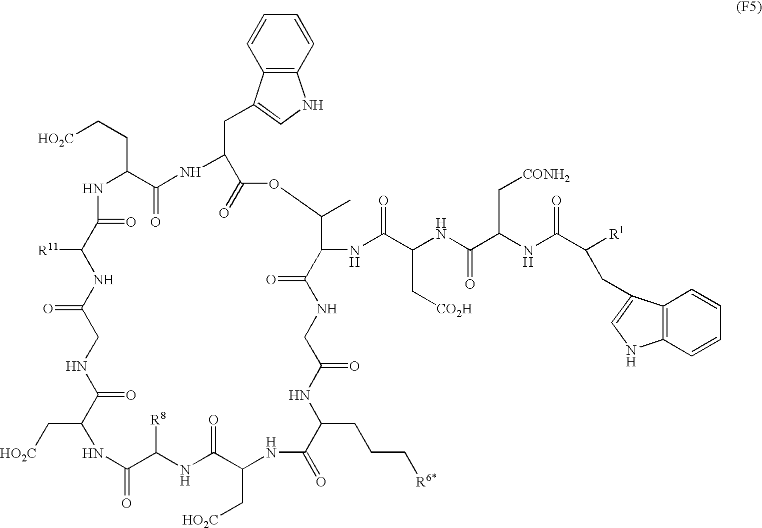
and salts thereof; wherein:
a) R⁸ is hydrogen, methyl, 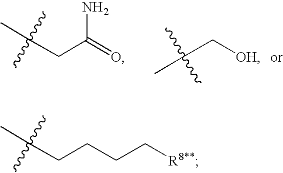
b) R¹¹ is methyl, 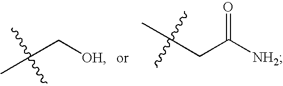
   and
c) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

The present invention provides, in another aspect, compounds of Formula F6: 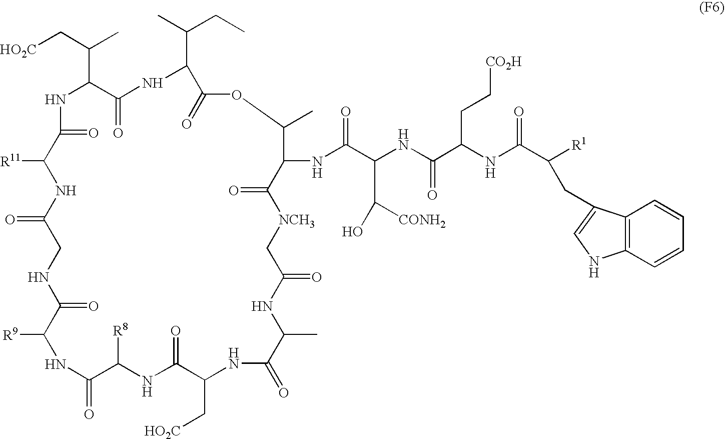
and salts thereof; wherein:
a) R⁸ is 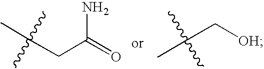
b) R⁹ is 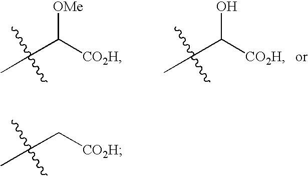
c) R¹¹ is, methyl, 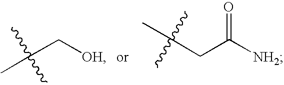
d) R¹² is H or CH₃; and
e) R¹ is amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides compounds of Formula F7: 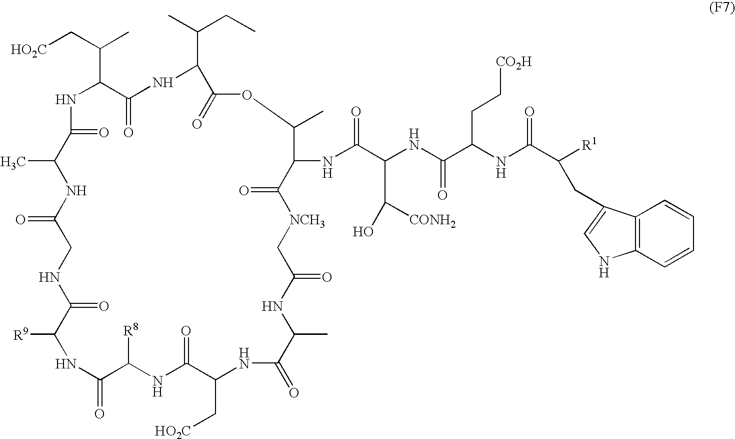
and salts thereof, wherein:
a) R⁸ is methyl, 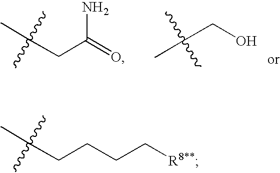
b) R⁹ is 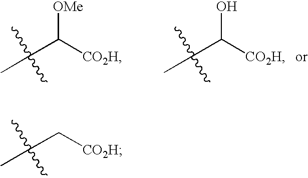
c) R¹² is H or CH₃; and
d) each of R¹ and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

The present invention provides, in another aspect, compounds of Formula F8: 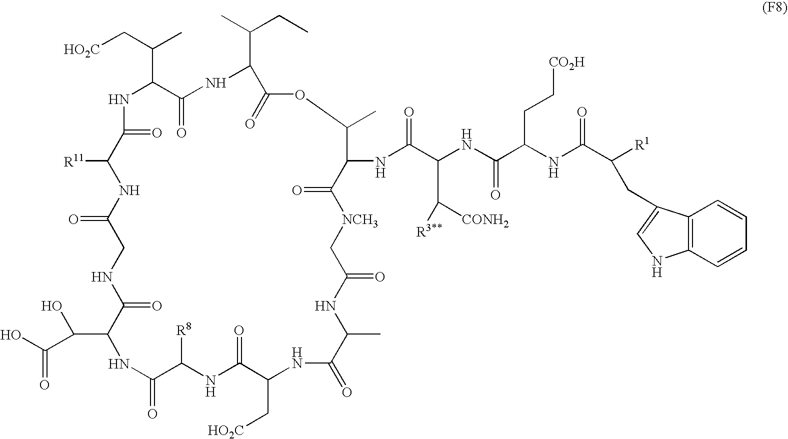
and salts thereof; wherein:
a) R³** is hydroxyl or hydrogen
b) R⁸ is methyl, 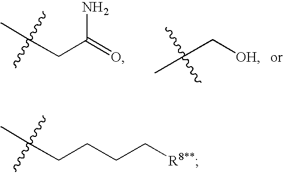
c) R¹¹ is an amino acid side chain, methyl, 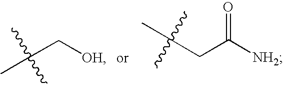
d) R¹² is H or CH₃; and
e) each of R¹ and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides compounds of Formula F9: 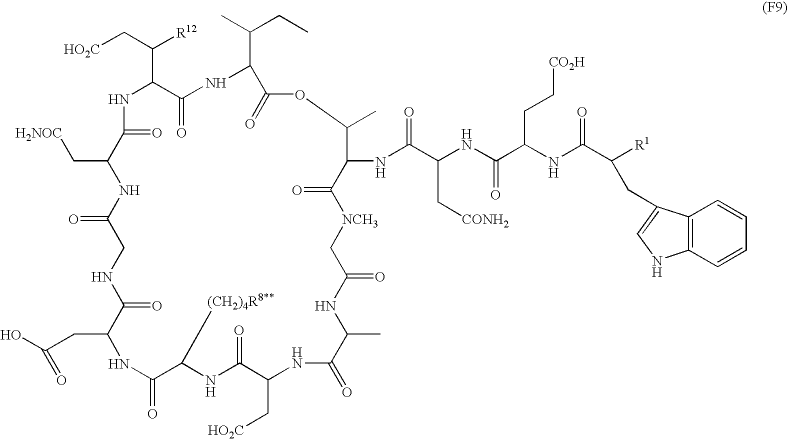
and salts thereof; wherein:
a) R¹² is H or CH₃; and
b) each of R¹, and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

The present invention provides, in another aspect, compounds of Formula F10: 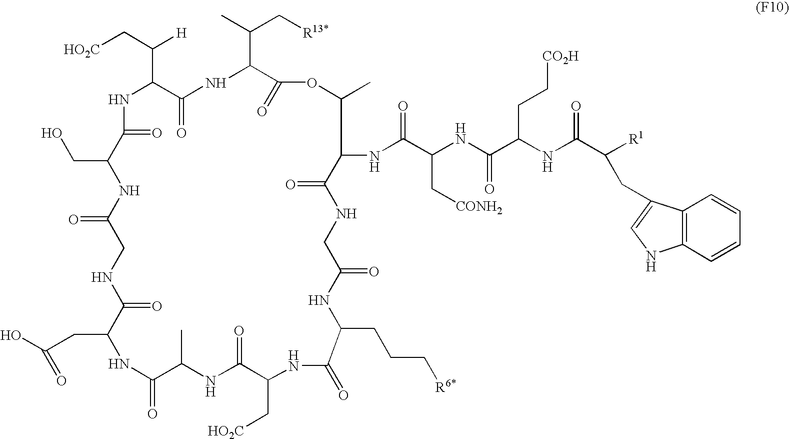
and salts thereof; wherein:
a) R¹³* is H or CH₃; and
b) each of R¹, and R⁶* is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides compounds of Formula F11: 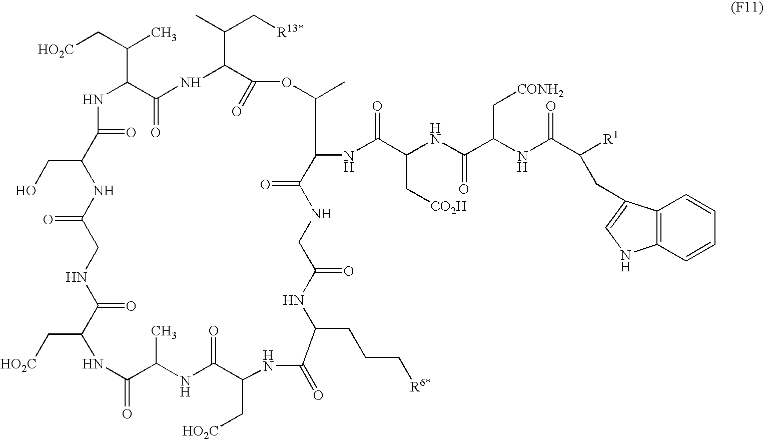
and the salts thereof; wherein:
a) R¹³* is H or CH₃; and
b) each of R⁶* is independently amino, monosubstituted amino, disubstituted amino, NH-amino protecting group, acylamino, ureido, guanidino, carbamoyl, sulfonamino, thioacylamino, thioureido, imino amino, or phosphonamino.

The present invention provides, in another aspect, compounds of Formula F12: 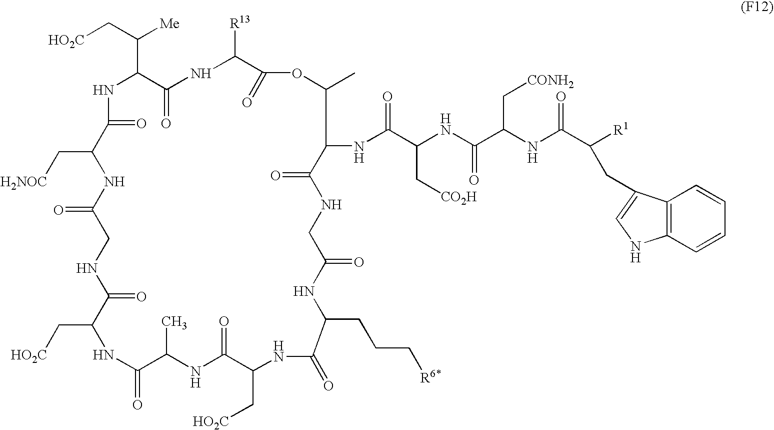
and salts thereof; wherein:
a) R¹³ is CH(CH₂CH₃)CH₃ or 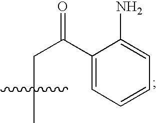
   and
b) each of R¹ and R⁶* is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides compounds of Formula F13: 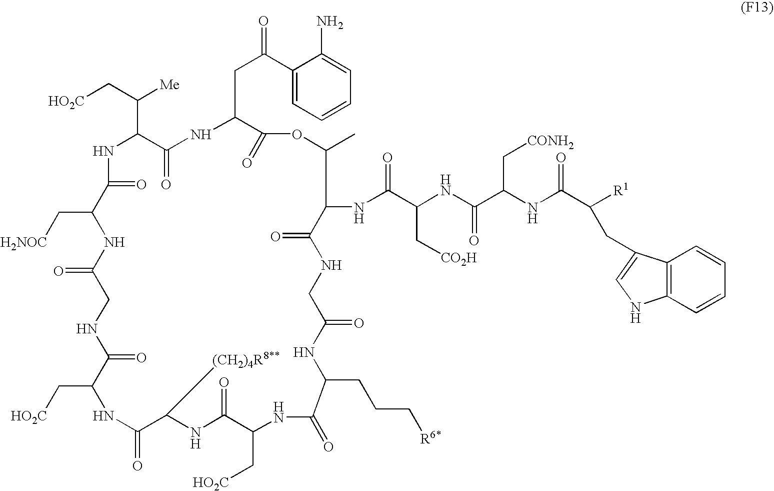
and salts thereof; wherein each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

The present invention provides, in another aspect, compounds of Formula F14: 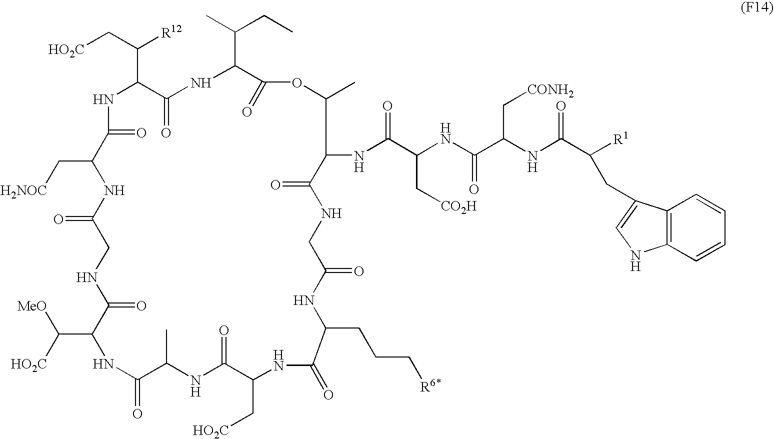
and salts thereof; wherein:
a) R¹² is H or CH₃; and
b) each of R¹ and R⁶* is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides compounds of Formula F15: 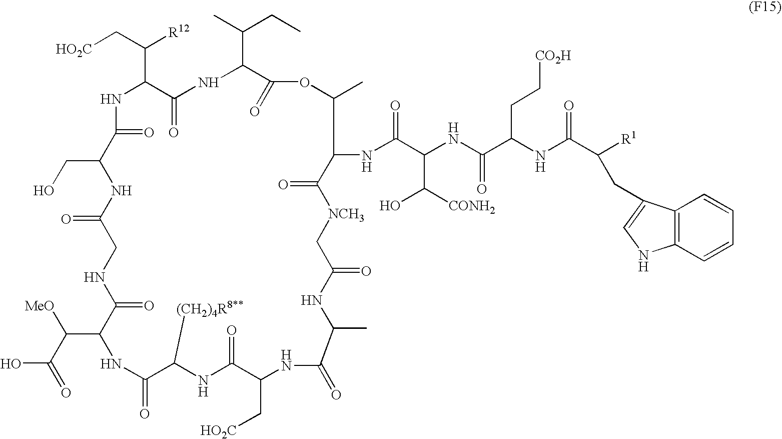
and salts thereof; wherein:
a) R¹² is H or CH₃; and
b) each of R¹ and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

The present invention provides, in another aspect, compounds of Formula F16: 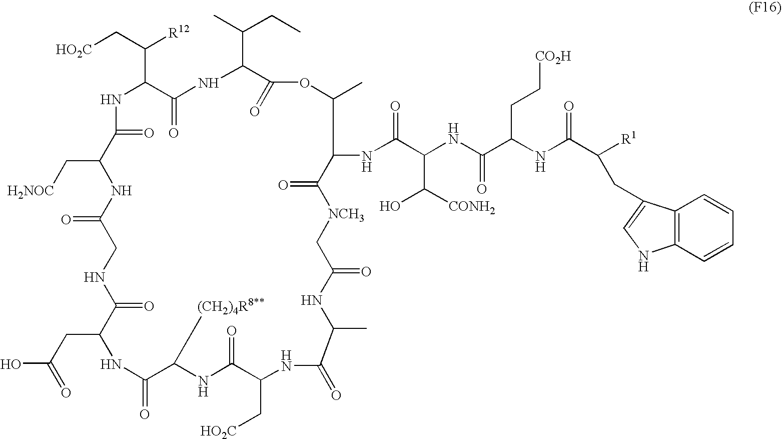
and the salts thereof; wherein:
a) R¹² is H or CH₃, and
b) each of R¹ and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides compounds of Formula F17: 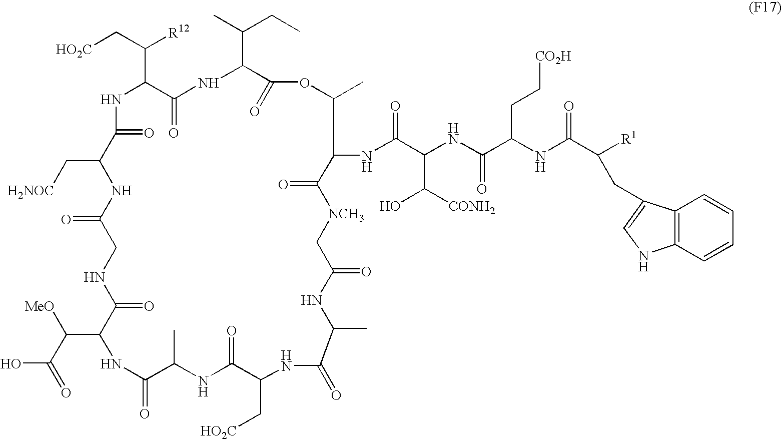
and salts thereof; wherein:
a) R¹² is H or CH₃; and
b) R¹ is amino, monosubstituted amino, disubstituted amino, NH-amino protecting group, acylamino, ureido, guanidino, carbamoyl, sulfonamino, thioacylamino, thioureido, iminoamino, or phosphonamino.

The present invention provides, in another aspect, compounds of Formula F18: 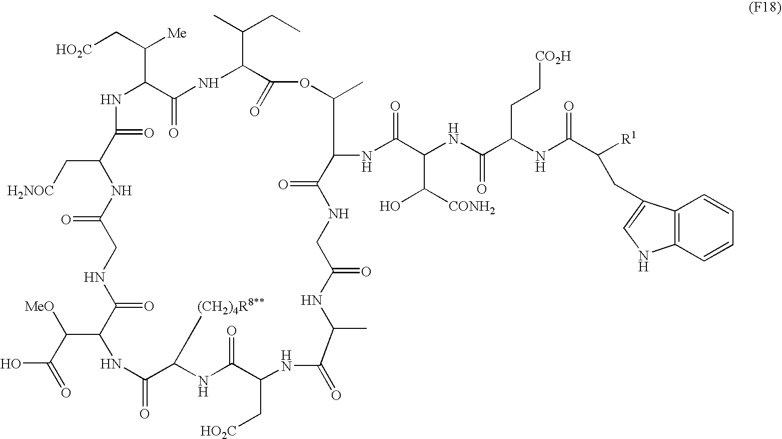
and salts thereof; wherein each of R¹ and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides compounds of Formula F19: 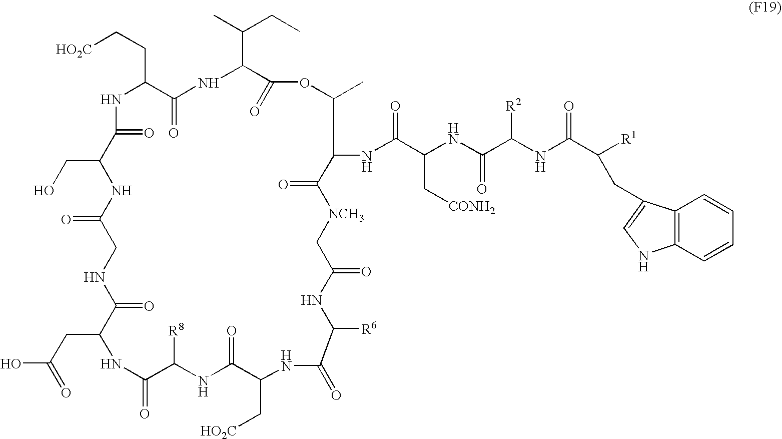
and salts thereof, wherein:
a) R² is 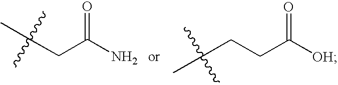
b) R⁶ is methyl or 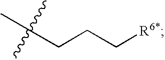
c) R⁸ is methyl or 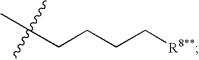
   and
d) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

The present invention provides, in another aspect, compounds of Formula F20: 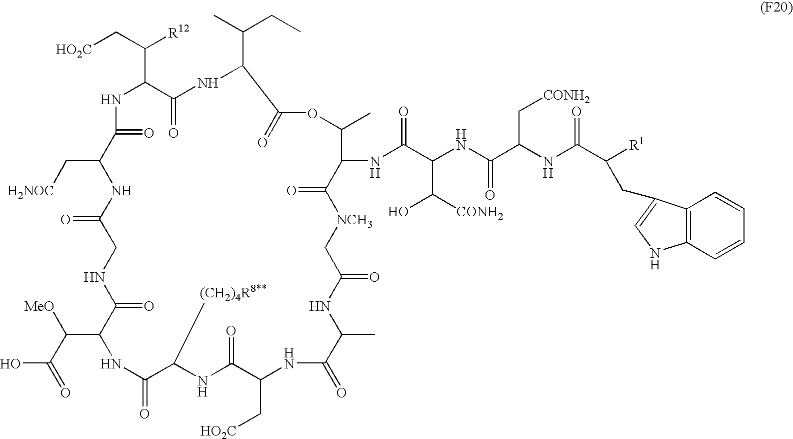
and salts thereof; wherein:
a) R¹² is H or CH₃; and
b) each of R¹ and R⁸** is amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides compounds of Formula F21 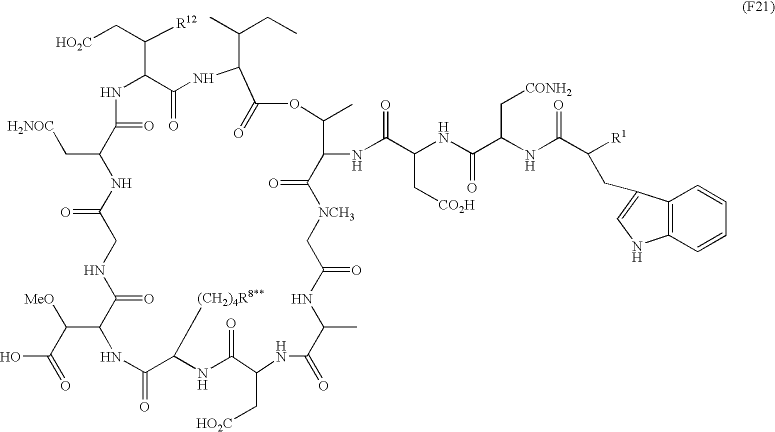
and salts thereof; wherein:
a) R¹ is 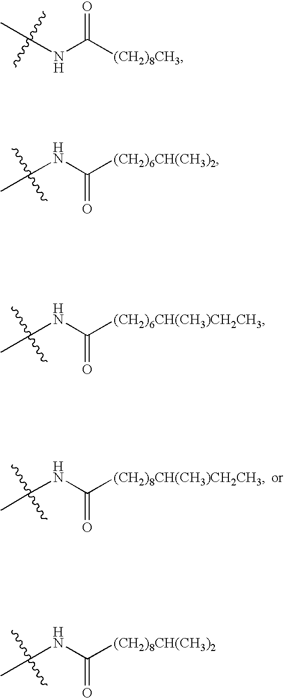
b) R¹² is H or CH₃, and
c) R⁸** is amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the invention provides compounds of Formula F22 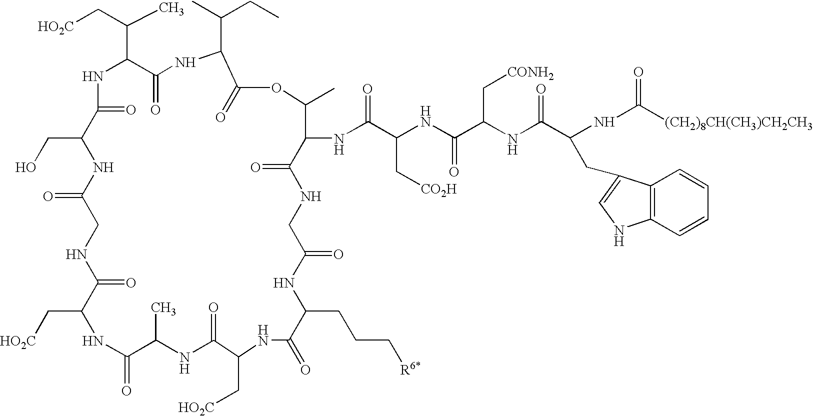
and salts thereof; wherein:
R⁶* is amino, monosubstituted amino, disubstituted amino, NH-amino protecting group, acylamino, ureido, guanidino, carbamoyl, sulfonamino, thioacylamino, thioureido, iminoamino, or phosphonamino.

In another aspect, the present invention also provides pharmaceutical compositions including compounds of Formula I and compounds of Formula F1-F22, and methods of use thereof.

In yet another aspect, the present invention also provides antibacterial compositions including compounds of Formula I and compounds of Formula F1-F22, and methods of use thereof.

In a further aspect the present invention provides a process for preparing the compounds of Formula I and compounds of Formula F1-F22.

### BRIEF DESCRIPTION OF THE DRAWINGS

FIG. 1 shows a depiction of the biosynthetic genes cluster for daptomycin, A54145, and CDA. The numbers in parenthesis denote the amino acid number. The following abbreviations are used: Trp: tryptophan; Asn: asparagine; Asp: aspartic acid; Thr: threonine; Gly: glycine; Orn: ornithine; Ala: alanine; Ser: serine; MeGlu: 3-methylglutamic acid; Kyn: kynurenine; Glu: glutamic acid; hAsn: 3-hydroxyasparagine; Sar: sarcosine; Lys: lysine; OMeAsp: 3-methoxyaspartic acid; Ile: isoleucine; Val: valine; D-HPG:D-hydroxyphenyl glycine.

FIG. 2 depicts the deletion of dptA-H in S. roseosporus whereby a dptA-H deletion was constructed in S. roseosporus, by exchanging the tsr (thiostrepton resistance) and cat (chloramphenicol) for the dptA-H locus to construct the deletion in the chromosome of S. roseosporus.

FIG. 3 depicts the general method for “Red-mediated” gene replacement in the daptomycin NRPS pathway. The bacteriophage λ-induced “hyper-recombination” state (the “Red” system or Red-mediated recombination) was used to construct both deletions within dptBC and to clone the replacement modules via a technique called “gap-repair”. Abbreviations: “C”, condensation domain; “Aₛₑᵣ”, adenylation domain for serine; “T”, thiolation domain; “E”, epimerase domain.

FIG. 4 depicts constructs from S. roseosporus combinatorial library.

FIG. 5 depicts the module organization in dptBC (internal module for a D-amino acid in dptBC) and the terminal amino acid module (kynurenine) in dptD associated with the thioesterase. C: is a condensation domain. Circles containing amino acid 3 letter codes are adenylation domains specific to the amino acid: Asn: asparagines; Ala: alanine; Asp: aspartic acid; 3MGlu: 3-methylglutamic acid; and Kyn: kynurenine. T is a thiolation domain. E is an epimerization domain. TE is a thioesterase domain.

### DETAILED DESCRIPTION OF THE INVENTION

### Definitions

The term “acyl” denotes a carbonyl radical attached to an alkyl, alkenyl, alkynyl, cycloalkyl, heterocycyl, aryl or heteroaryl group, examples including, without limitation, such radicals as 8-methyldecanoyl, 10-methylundecanoyl, 10-methyldodecanoyl, n-decanoyl, 8-methylnonanoyl, dodecanoyl, undecanoyl, acetyl and benzoyl. In one embodiment of the invention, the acyl group is an “alkanoyl” group which is defined as a carbonyl radical attached to an alkyl group. In another embodiment of the invention, the alkanoyl group is a “C₁-C₂₀-alkanoyl” group which is defined as an alkanoyl group containing a total of 1 to 20 carbon atoms, including the carbonyl carbon. The carbon atoms can be arranged in a straight chain or branched chain. In another embodiment of the invention, the alkanoyl group is a “C₁-C₁₅-alkanoyl” group which is defined as an alkanoyl group containing a total of 1 to 15 carbon atoms, including the carbonyl carbon. The carbon atoms can be arranged in a straight chain or branched chain. In another embodiment of the invention, the alkanoyl group is a “C₁-C₁₃-alkanoyl” group which is defined as an alkanoyl group containing a total of 1 to 13 carbon atoms, including the carbonyl carbon. The carbon atoms can be arranged in a straight chain or branched chain. In another embodiment of the invention, the alkanoyl group is a “C₅-C₂₀-alkanoyl” group which is defined as an alkanoyl group containing a total of 5 to 20 carbon atoms, including the carbonyl carbon. The carbon atoms can be arranged in a straight chain or branched chain. In another embodiment of the invention, the alkanoyl group is a “C₁₀-C₂₀-alkanoyl” group which is defined as an alkanoyl group containing a total of 10 to 20 carbon atoms, including the carbonyl carbon. The carbon atoms can be arranged in a straight chain or branched chain. In another embodiment of the invention, the alkanoyl group is a “C₁₀-C₁₃-alkanoyl” group which is defined as an alkanoyl group containing a total of 1 to 13 carbon atoms, including the carbonyl carbon. The carbon atoms can be arranged in a straight chain or branched chain. In another embodiment of the invention, the alkanoyl group is 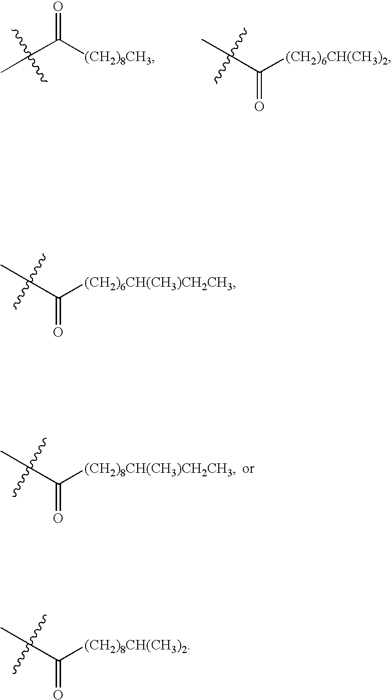
In another embodiment of the invention, the subsets of the term acyl are (1) “unsubstituted alkanoyl” which is defined as carbonyl radical attached to an unsubstituted alkyl group and (2) “unsubstituted alkenoyl” which is defined as carbonyl radical attached to an unsubstituted alkenyl group.

The term “acylamino” is defined as a nitrogen radical adjacent to an acyl group. In one embodiment of the invention, the acylamino group is an “alkanoylamino” group which is defined as a nitrogen radical attached to an alkanoyl group. In another embodiment of the invention, the alkanoylamino group is a “C₁-C₂₀-alkanoylamino” group which is defined as a alkanoylamino group containing a total of 1 to 20 carbon atoms, including the carbonyl carbon. The carbon atoms can be arranged in a straight chain or branched chain. In another embodiment of the invention, the alkanoylamino group is a “C₁-C₁₅-alkanoylamino” group which is defined as an alkanoylamino group containing a total of 1 to 15 carbon atoms, including the carbonyl carbon. The carbon atoms can be arranged in a straight chain or branched chain. In another embodiment of the invention, the alkanoylamino group is a “C₁-C₁₃-alkanoylamino” group which is defined as an alkanoylamino group containing a total of 1 to 13 carbon atoms, including the carbonyl carbon. The carbon atoms can be arranged in a straight chain or branched chain. In another embodiment of the invention, the alkanoylamino group is a “C₅-C₂₀-alkanoylamino” group which is defined as a alkanoylamino group containing a total of 5 to 20 carbon atoms, including the carbonyl carbon. The carbon atoms can be arranged in a straight chain or branched chain. In another embodiment of the invention, the alkanoylamino group is a “C₁₀-C₂₀-alkanoylamino” group which is defined as an alkanoylamino group containing a total of 10 to 20 carbon atoms, including the carbonyl carbon. The carbon atoms can be arranged in a straight chain or branched chain. In another embodiment of the invention, the alkanoylamino group is a “C₁₀-C₁₃-alkanoylamino” group which is defined as an alkanoylamino group containing a total of 1 to 13 carbon atoms, including the carbonyl carbon. The carbon atoms can be arranged in a straight chain or branched chain. In another embodiment of the invention, the alkanoylamino group is

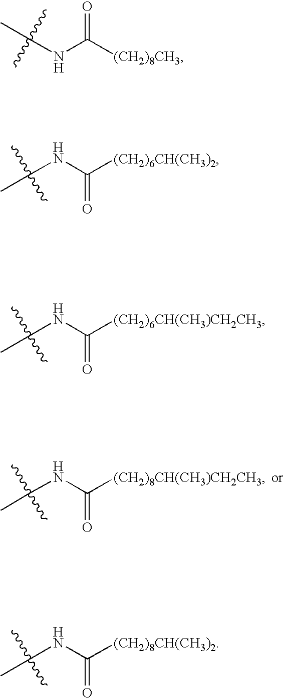

The term “acyloxy” denotes an oxygen radical adjacent to an acyl group.

The term “alkenyl” is defined as linear or branched radicals having two to about twenty carbon atoms, preferably three to about ten carbon atoms, and containing at least one carbon-carbon double bond. One or more hydrogen atoms can also be replaced by a substituent group selected from acyl, acylamino, acyloxy, alkenyl, alkoxy, alkyl, alkynyl, amino, aryl, aryloxy, carbamoyl, carboalkoxy, carboxy, carboxyamido, carboxyamino, cyano, disubstituted amino, formyl, guanidino, halo, heteroaryl, heterocyclyl, hydroxy, iminoamino, monosubstituted amino, nitro, oxo, phosphonamino, sulfinyl, sulfonamino, sulfonyl, thio, thioacylamino, thioureido, or ureido. The double bond portion(s) of the unsaturated hydrocarbon chain may be either in the cis or trans configuration. Examples of alkenyl groups include, without limitation, ethylenyl or phenyl ethylenyl. A subset of term alkenyl is “unsubstituted alkenyl” which is defined as an alkenyl group that bears no substituent groups.

The term “alkoxy” denotes oxygen radical substituted with an alkyl, cycloalkyl or heterocyclyl group. Examples include, without limitation, methoxy, tert-butoxy, benzyloxy and cyclohexyloxy.

The term “alkyl” is defined as a linear or branched, saturated radical having one to about twenty carbon atoms unless otherwise specified. The term “lower alkyl” is defined as an alkyl group containing 1-4 carbon atoms. One or more hydrogen atoms can also be replaced by a substitutent group selected from acyl, acylamino, acyloxy, alkenyl, alkoxy, alkyl, alkynyl, amino, aryl, aryloxy, carbamoyl, carboalkoxy, carboxy, carboxyamido, carboxyamino, cyano, disubstituted amino, formyl, guanidino, halo, heteroaryl, heterocyclyl, hydroxy, iminoamino, monosubstituted amino, nitro, oxo, phosphonamino, sulfinyl, sulfonamino, sulfonyl, thio, thioacylamino, thioureido, or ureido. Examples of alkyl groups include, without limitation, methyl, butyl, tert-butyl, isopropyl, trifluoromethyl, nonyl, undecyl, octyl, dodecyl, methoxymethyl, 2-(2′-aminophenacyl), 3-indolylmethyl, benzyl, and carboxymethyl. Subsets of the term alkyl are (1) “unsubstituted alkyl” which is defined as an alkyl group that bears no substituent groups and (2) “substituted alkyl” which denotes an alkyl radical in which one or more hydrogen atoms is replaced by a substitutent group selected from acyl, acylamino, acyloxy, alkenyl, alkoxy, alkyl, alkynyl, amino, aryl, aryloxy, carbamoyl, carboalkoxy, carboxy, carboxyamido, carboxyamino, cyano, disubstituted amino, formyl, guanidino, halo, heteroaryl, heterocyclyl, hydroxy, iminoamino, monosubstituted amino, nitro, oxo, phosphonamino, sulfinyl, sulfonamino, sulfonyl, thio, thioacylamino, thioureido, or ureido. In another embodiment of the invention, the alkyl group is a “C₁-C₂₀-alkyl” group which is defined as a alkyl group containing a total of 1 to 20 carbon atoms. The carbon atoms can be arranged in a straight chain or branched chain. In another embodiment of the invention, the alkyl group is a “C₁-C₁₅-alkyl” group which is defined as a alkyl group containing a total of 1 to 15 carbon atoms. The carbon atoms can be arranged in a straight chain or branched chain. In another embodiment of the invention, the alkyl group is a “C₁-C₁₃-alkyl” group which is defined as an alkyl group containing a total of 1 to 13 carbon atoms. The carbon atoms can be arranged in a straight chain or branched chain. In another embodiment of the invention, the alkyl group is a “C₅-C₂₀-alkanoyl” group which is defined as a alkyl group containing a total of 5 to 20 carbon atoms. The carbon atoms can be arranged in a straight chain or branched chain. In another embodiment of the invention, the alkyl group is a “C₁₀-C₂₀-alkyl” group which is defined as a alkyl group containing a total of 10 to 20 carbon atoms. The carbon atoms can be arranged in a straight chain or branched chain. In another embodiment of the invention, the alkyl group is a “C₁₀-C₁₃-alkyl” group which is defined as a alkyl group containing a total of 10 to 13 carbon atoms. In another embodiment of the invention, the alkyl group is a “C₉-C₁₂-alkyl” group which is defined as a alkyl group containing a total of 9 to 12 carbon atoms. The carbon atoms can be arranged in a straight chain or branched chain. In another embodiment of the invention, the alkyl group is nonyl, 7-methyloctyl, 7-methylnonyl, n-decyl, 9-methylundecyl, 9-methyldecyl, n-undecyl.

The term “alkylidenyl” is defined as a carbon radical of the formula 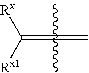
wherein R^{x} and R^{x1} are independently selected from hydrido or C₇-C₁₇ unsubstituted alkyl, wherein the total number of carbons from R^{x} and R^{x1} does not exceed 17.

The term “alkynyl” denotes linear or branched radicals having from two to about ten carbon atoms, and containing at least one carbon-carbon triple bond. One or more hydrogen atoms can also be replaced by a substituent group selected from acyl, acylamino, acyloxy, alkenyl, alkoxy, alkyl, alkynyl, amino, aryl, aryloxy, carbamoyl, carboalkoxy, carboxy, carboxyamido, carboxyamino, cyano, disubstituted amino, formyl, guanidino, halo, heteroaryl, heterocyclyl, hydroxy, iminoamino, monosubstituted amino, nitro, oxo, phosphonamino, sulfinyl, sulfonamino, sulfonyl, thio, thioacylamino, thioureido, or ureido. An example of alkynyl group includes, without limitation, propynyl.

The term “amino” is defined as an NH₂ radical.

The term “amino acid” denotes a compound of the formula

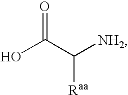

wherein R^{aa} is an amino acid side chain. A “naturally occurring amino acid” is an amino acid that is found in nature. An “essential amino acid” is one of the twenty common amino acids: alanine, arginine, asparagine, aspartic acid, cysteine, glutamine, glutamic acid, glycine, histidine, isoleucine, leucine, lysine, methionine, phenyalanine, proline, serine, threonine, tryptophan, tyrosine and valine. A “non-proteinogenic amino acid” is any amino acid other than an essential amino acid. In this specification, the following abbreviations are used to describe specific amino acids:

| Abbreviation(s) | Amino acid |
|---|---|
| (MeO)Asp or (m)Asp or mAsp | 3-methoxy-aspartic acid |
| or moAsp or mo(Asp) | |
| (OH)Asn or h(Asn) or hAsn or | 3-hydroxy-asparagine |
| h-Asn | |
| (OH)Asp or h(Asp) or hAspor | 3-hydroxy-aspartic acid |
| h-Asp | |
| 3-MG | 3-methylglutamic acid |
| D-HPG | D-hydroxyphenyl glycine |
| Ala | Alanine |
| Asn | Asparagines |
| Asp | Aspartic acid |
| Glu | Glutamic acid |
| Gly | Glycine |
| Ile | Isoleucine |
| Kyn | Kynurinine |
| Lys | Lysine |
| Orn | Ornithine |
| Sar | Sarcosine |
| Ser | Serine |
| Thr | Threonine |
| Trp | Tryptophan |
| Val | Valine |

In one aspect of the invention amino acids are 3-methoxy-aspartic acid, 3-hydroxy-asparagine, 3-hydroxy-aspartic acid, 3-methylglutamic acid, Alanine, Asparagine, Aspartic acid, Glutamic acid, Glycine, Isoleucine, Kynurinine, Lysine, Ornithine, Sarcosine, Serine, Threonine, Tryptophan, and Valine.

It will be understood by those of skill in the art, that peptides are described by the joining of the three letter codes above. For example, Asp-Asn-Trp refers to the compound 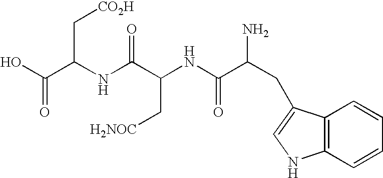
Alternatively, the compound above could also be described as Asp-Asn-Trp-NH₂. It will also be understood by one of skill in the art that the peptides of the invention may contain protecting groups (vide infra). When an amino acid contains a protecting group, the three letter code will be adapted to indicate the protecting group. For example, Thr-Asp(OtBu)-Asn(NHTrt)-Trp-NH₂, refers to the following compound:

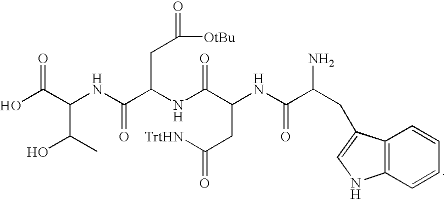

Common protecting groups for the amino acids of this invention include tert-butoxy (tBu), trityl (Trt) and tert-butoxy carbonyl (BOC) protecting groups.

It will also be understood by one of skill in the art that cyclic peptides may also be described by three letter codes. For example, the three letter structure 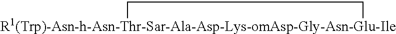
is identical with the structure:

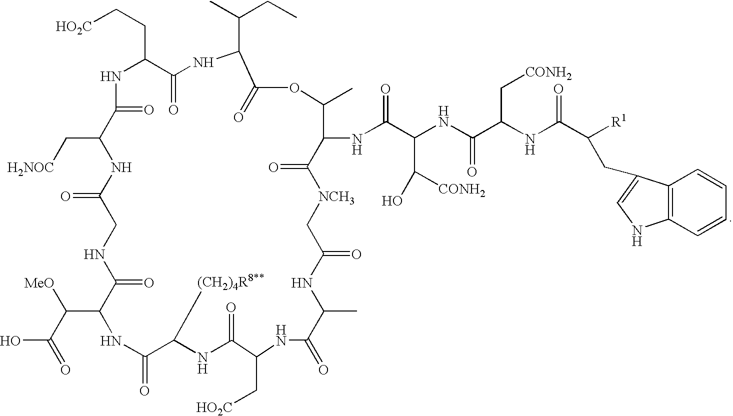

It will also be understood by one of skill in the art that amino acids can exist in either the L or D configuration. When it is desirable to indicate the configuration of the amino acid, the D or L designation is placed before the three letter code.

The term “amino acid residue” denotes a compound of the formula 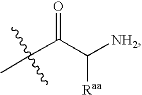
wherein R^{aa} is an amino acid side chain. In one aspect of the invention, the amino acid residue is derived from a natural amino acid. In another aspect of the invention, the amino acid residue is derived from the amino acids 3-methoxy-aspartic acid, 3-hydroxy-asparagine, 3-hydroxy-aspartic acid, 3-methylglutamic acid, Alanine, Asparagine, Aspartic acid, Glutamic acid, Glycine, Isoleucine, Kynurinine, Lysine, Ornithine, Sarcosine, Serine, Threonine, Tryptophan, and Valine.

The term “amino acid side chain” denotes any side chain (R group) from a naturally-occurring or synthetic amino acid. For example, 3-indolylmethyl could also be called a tryptophan side chain. Examples of amino acid side chains include, without limitation, 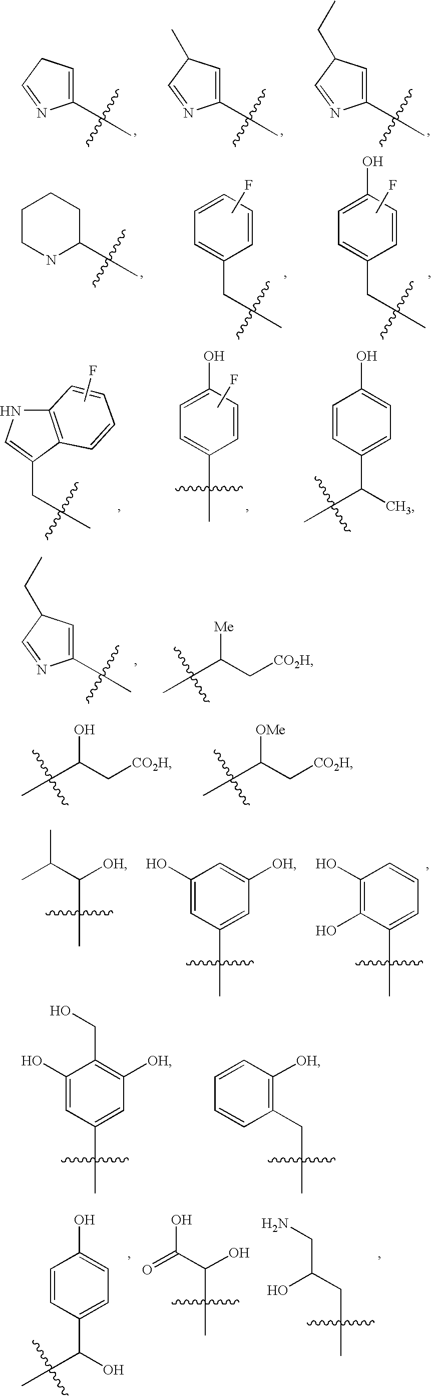 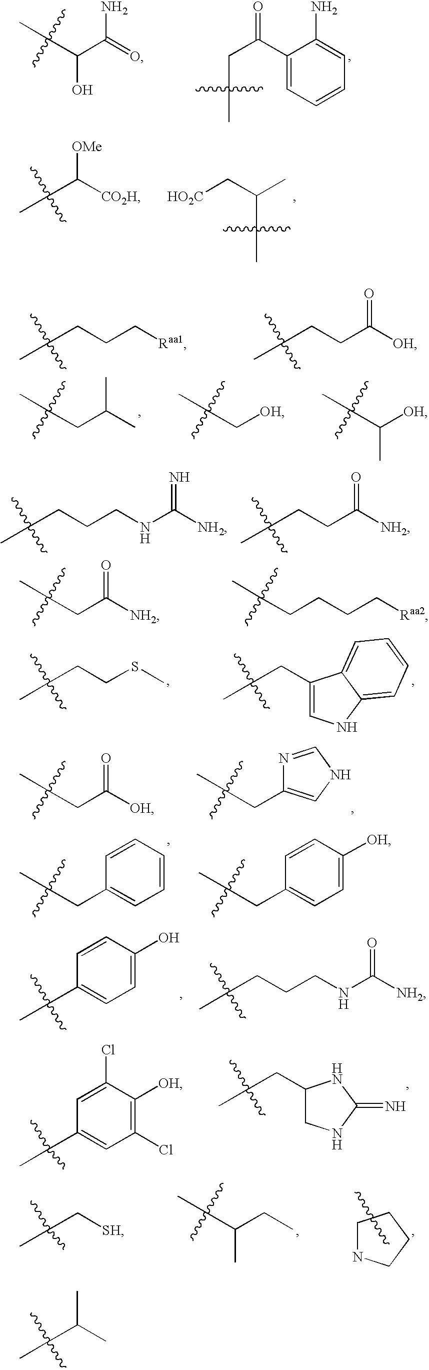
hydrido and methyl, wherein each of R^{aa1} and R^{aa2} is independently amino, monosubstituted amino, disubstituted amino, acylamino, ureido, guanidino, carbamoyl, sulfonamino, thioacylamino, thioureido, iminoamino, or phosphonamino. A “non-proteinogenic amino acid side chain” is an amino acid side chain derived from a non-proteinogenic amino acid (vide supra). Examples of a non-proteinogenic amino acid side chains include, without limitation, 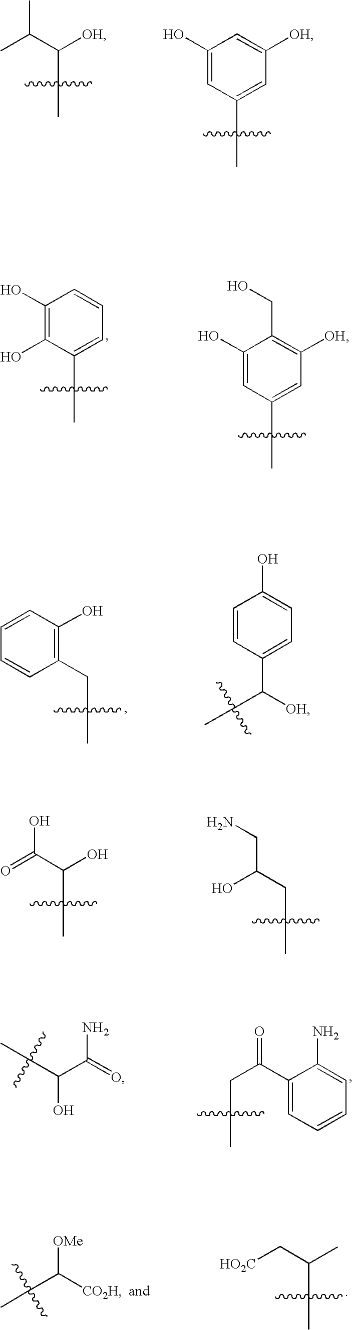
In one aspect of the invention, the amino acid side chain is derived from a natural amino acid. In another aspect of the invention, the amino acid side chain is derived from the amino acids 3-methoxy-aspartic acid, 3-hydroxy-asparagine, 3-hydroxy-aspartic acid, 3-methylglutamic acid, Alanine, Asparagine, Aspartic acid, Glutamic acid, Glycine, Isoleucine, Kynurinine, Lysine, Ornithine, Sarcosine, Serine, Threonine, Tryptophan, and Valine.

The term “2-(2′-aminophenacyl)” refers to a radical of the formula

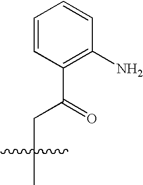

The term “aryl” or “aryl ring” is defined as an aromatic radical in a single or fused carbocyclic ring system, having from five to fourteen ring members. In a preferred embodiment, the ring system has from six to ten ring members. One or more hydrogen atoms may also be replaced by a substituent group selected from acyl, acylamino, acyloxy, alkenyl, alkoxy, alkyl, alkynyl, amino, aryl, aryloxy, azido, carbamoyl, carboalkoxy, carboxy, carboxyamido, carboxyamino, cyano, disubstituted amino, formyl, guanidino, halo, heteroaryl, heterocyclyl, hydroxy, iminoamino, monosubstituted amino, nitro, oxo, phosphonamino, sulfinyl, sulfonamino, sulfonyl, thio, thioacylamino, thioureido, or ureido. Examples of aryl groups include, without limitation, phenyl, naphthyl, biphenyl, terphenyl.

The term “aryloxy” denotes oxy-containing radicals substituted with an aryl or heteroaryl group. Examples include, without limitation, phenoxy.

The term “carbamoyl” denotes a nitrogen radical of the formula 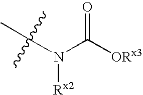
wherein R^{x2} is selected from hydrido, alkyl, aryl, cycloalkyl, heteroaryl or heterocyclyl and R^{x3} is selected from alkyl, aryl, cycloalkyl, heteroaryl or heterocyclyl.

The term “carboalkoxy” is defined as a carbonyl radical adjacent to an alkoxy or aryloxy group.

The term “carboxy” denotes a COOH radical.

The term “carboxyamino” denotes a CONH₂ radical.

The term “carboxyamido” is defined as a carbonyl radical adjacent to a monosubstituted amino or disubstituted amino group.

The term “α-carboxy amino acid side chain” is defined as a carbon radical of the formula 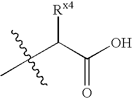
wherein R^{x4} is defined as an amino acid side chain.

The term “carboxymethyl” denotes a CH₂CO₂H radical.

The term “cycloalkyl” or “cycloalkyl ring” denotes a saturated or partially unsaturated carbocyclic ring in a single or fused carbocyclic ring system having from three to twelve ring members. In a preferred embodiment, a cycloalkyl is a ring system having three to seven ring members. One or more hydrogen atoms may also be replaced by a substituent group selected from acyl, acylamino, acyloxy, alkenyl, alkoxy, alkyl, alkynyl, amino, aryl, aryloxy, carbamoyl, carboalkoxy, carboxy, carboxyamido, carboxyamino, cyano, disubstituted amino, formyl, guanidino, halo, heteroaryl, heterocyclyl, hydroxy, iminoamino, monosubstituted amino, nitro, oxo, phosphonamino, sulfinyl, sulfonamino, sulfonyl, thio, thioacylamino, thioureido, or ureido. Examples of a cycloalkyl group include, without limitation, cyclopropyl, cyclobutyl, cyclohexyl, and cycloheptyl.

The term “disubstituted amino” is defined as a nitrogen radical containing two substituent groups independently selected from, alkyl, cycloalkyl, heterocyclyl, aryl, or heteroaryl. Preferred disubstituted amino radicals are “lower disubstituted amino” radicals, whereby the substituent groups are lower alkyl. Also preferred disubstituted amino radicals are amino radicals wherein one substituent is a lower alkyl group and the other substituent is an α-carboxy amino acid side chain.

The group “Fmoc” is a 9-fluorenylmethoxycarbonyl group.

The term “guanidino” is defined as a nitrogen radical of the formula 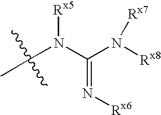
wherein each of R^{x5}, R^{x7} and R^{x8} is independently selected from hydrido, alkyl, aryl, cycloalkyl, heteroaryl or heterocyclyl group; and R^{x6} is selected from alkyl, aryl, cycloalkyl, heteroaryl or heterocyclyl group.

The term “halo” denotes a bromo, chloro, fluoro or iodo radical.

“Heteroaryl” or “heteroaryl ring” is defined as an aromatic radical which contain one to four hetero atoms or hetero groups selected from O, N, S, or SO in a single or fused heterocyclic ring system, having from five to fifteen ring members. In a preferred embodiment, the heteroaryl ring system has from six to ten ring members. One or more hydrogen atoms may also be replaced by a substituent group selected from acyl, acylamino, acyloxy, alkenyl, alkoxy, alkyl, alkynyl, amino, aryl, aryloxy, carbamoyl, carboalkoxy, carboxy, carboxyamido, carboxyamino, cyano, disubstituted amino, formyl, guanidino, halo, heteroaryl, heterocyclyl, hydroxy, iminoamino, monosubstituted amino, nitro, oxo, phosphonamino, sulfinyl, sulfonamino, sulfonyl, thio, thioacylamino, thioureido, or ureido. Examples of heteroaryl groups include, without limitation, pyridinyl, thiazolyl, thiadiazoyl, isoquinolinyl, pyrazolyl, oxazolyl, oxadiazoyl, triazolyl, and pyrrolyl groups.

The term “heterocyclyl,” “heterocyclic” or “heterocyclyl ring” denotes a saturated or partially unsaturated ring containing one to four hetero atoms or hetero groups selected from O, N, NH, N(lower alkyl), S, SO or SO₂, in a single or fused heterocyclic ring system having from three to twelve ring members. In a preferred embodiment, a heterocyclyl is a ring system having three to seven ring members. One or more hydrogen atoms may also be replaced by a substituent group selected from acyl, acylamino, acyloxy, alkenyl, alkoxy, alkyl, alkynyl, amino, aryl, aryloxy, carbamoyl, carboalkoxy, carboxy, carboxyamido, carboxyamino, cyano, disubstituted amino, formyl, guanidino, halo, heteroaryl, heterocyclyl, hydroxy, iminoamino, monosubstituted amino, nitro, oxo, phosphonamino, sulfinyl, sulfonamino, sulfonyl, thio, thioacylamino, thioureido, or ureido. Examples of a heterocyclyl group include, without limitation, morpholinyl, piperidinyl, and pyrrolidinyl.

The term “hydrido” is defined as a single hydrogen atom (H).

The term “iminoamino” denotes a nitrogen radical of the formula: 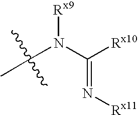
wherein each of R^{x9} and R^{x11} is independently selected from a hydrido, alkyl, cycloalkyl, aryl, heteroaryl or heterocyclyl group; and R^{x10} is selected from an alkyl, cycloalkyl, aryl, heteroaryl or heterocyclyl group.

The term “N-methylamino acid” denotes a compound of the formula 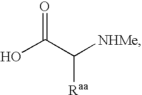
wherein R^{aa} is an amino acid side chain. Examples of amino acid side chains of an N-methyl amino acid include

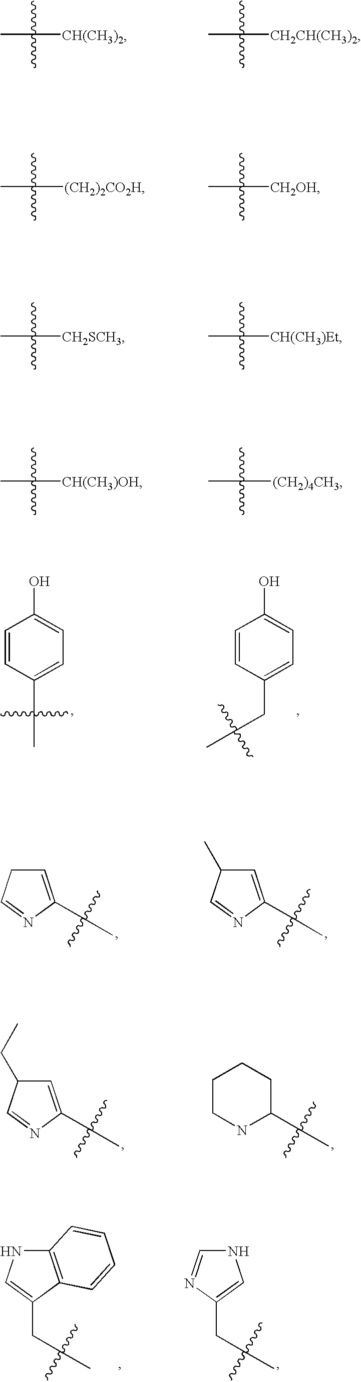

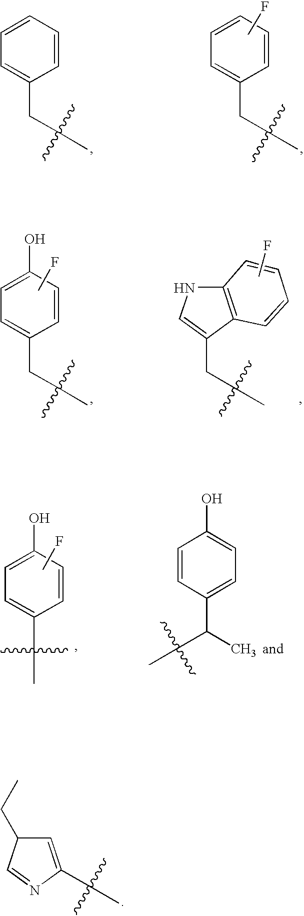

The term “monosubstituted amino” denotes a nitrogen radical containing a hydrido group and a substituent group selected from alkyl, cycloalkyl, heterocyclyl, aryl, or heteroaryl. Preferred monosubstituted amino radicals are “lower monosubstituted amino” radicals, whereby the substituent group is a lower alkyl group. More preferred monosubstituted amino radicals are amino radicals containing an α-carboxy amino acid side chain.

The term “phosphonamino” is defined as a nitrogen radical of the formula: 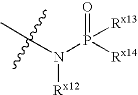
wherein R^{x12} is selected from hydrido, alkyl, aryl, cycloalkyl, heteroaryl or heterocyclyl; wherein each of R^{x13} and R^{x14} is independently selected from alkyl, alkoxy, aryl, aryloxy, cycloalkyl, heteroaryl and heterocyclyl.

The term “protecting group” refers to any chemical compound that may be used to prevent a group on a molecule from undergoing a chemical reaction while chemical change occurs elsewhere in the molecule. Groups that may need protecting include hydroxyl, amino, carboxylic acids and carboxyamino groups. Numerous protecting groups are known to those skilled in the art and examples can be found in “Protective Groups in Organic Synthesis” by Theodora W. Greene and Peter G. M. Wuts, John Wiley and Sons, New York, 3^{rd} Edition 1999, hereafter Greene.

The term “amino protecting group” refers to any chemical compound that may be used to prevent an amino group on a molecule from undergoing a chemical reaction while chemical change occurs elsewhere in the molecule. Numerous amino protecting groups are known to those skilled in the art and examples can be found in Greene. Examples of “amino protecting groups” include phthalimido, trichloroacetyl, STA-base, benzyloxycarbonyl, t-butoxycarbonyl, t-amyloxycarbonyl, isobornyloxycarbonyl, adamantyloxycarbonyl, chlorobenzyloxycarbonyl, nitrobenzyloxycarbonyl or the like. Preferred amino protecting groups are “carbamate amino protecting groups” which are defined as an amino protecting group that when bound to an amino group forms a carbamate, or the azido group. Preferred amino carbamate protecting groups are allyloxycarbonyl (alloc), carbobenzyloxy (CBZ), 9-fluorenylmethoxycarbonyl (Fmoc) and tert-butoxycarbonyl protecting groups.

The term “hydroxyl protecting group” refers to any chemical compound that may be used to prevent a hydroxyl group on a molecule from undergoing a chemical reaction while chemical change occurs elsewhere in the molecule. Numerous hydroxyl protecting groups are known to those skilled in the art and examples can be found in Greene (vide supra) Examples of hydroxyl protecting groups include esters such as, but not limited to formate, acetate, substituted acetate, crotonate, benzoate, substituted benzoates, methyl carbonate, ethyl carbonate, alkyl and aryl carbonates, borates, and sulphonates. Examples of hydroxyl protecting groups also include ethers such as, but not limited to methyl, benzyloxylmethyl, siloxymethyl, tetrahydropyranyl, substituted tetrahydropyranyl, ethyl, substituted ethyl, allyl, tert-butyl, propargyl, phenyl, substituted phenyl, benzyl, substituted benzyl, alkyl silyl and silyl ethers or the like. Preferred hydroxyl protecting groups are “acid labile ethers” which are defined as an ether protecting group that may be removed by treatment with acid. Preferred hydroxyl ether protecting groups are trityl (Trt), tert-butyl (tBu), benzyl (Bzl) and tert-butyldimethylsilyl (TBDMS) protecting groups.

The term “carboxylic acid protecting group” refers to any chemical compound that may be used to prevent a carboxylic acid on a molecule from undergoing a chemical reaction while chemical change occurs elsewhere in the molecule. Numerous carboxylic acid protecting groups are known to those skilled in the art and examples can be found in Greene (vide supra). Examples of carboxylic acid protecting groups include, but are not limited to, amides, hydrazides, and esters such as, methyl esters, substituted methyl, phenacyl, tetrahydropyranyl, tetrahydrofuranyl, cyanomethyl, triisopropylsilylmethyl, desyl, ethyl 2-substituted ethyl, phenyl, 2,6 dialkyl phenyl, benzyl, substituted benzyl, silyl, and stannyl, or the like. Preferred carboxylic acid ester protecting groups are allyl (All), tert-butyl (tBu), benzyl (Bzl), 4-{N-[1-(4,4-dimethyl-2,6-dioxocyclohexylidinene)-3-methylbutyl]-amino}benzyl (ODmab), 1-adamantyl (1Ada) and 2-phenylisopropyl (2-PhiPr) protecting groups.

The term “sulfinyl” denotes a tetravalent sulfur radical substituted with an oxo substituent and a second substituent selected from the group consisting of alkyl, cycloalkyl, heterocyclyl, aryl, or heteroaryl group.

The term sulfonamino is defined as an amino radical of the formula: 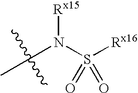
wherein R^{x15} is selected from a hydrido, alkyl, cycloalkyl, aryl, heteroaryl or heterocyclyl group; and R^{x16} is selected from alkyl, cycloalkyl, aryl, heteroaryl or heterocyclyl group.

The term “sulfonyl” denotes a hexavalent sulfur radical substituted with two oxo substituents and a third substituent selected from alkyl, cycloalkyl, heterocyclyl aryl, or heteroaryl.

The term “thio” is defined as a radical containing a substituent group independently selected from hydrido, alkyl, cycloalkyl, heterocyclyl, aryl and heteroaryl, attached to a divalent sulfur atom, such as, methylthio and phenylthio.

The term “thioacylamino” denotes an amino radical of the formula 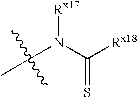
wherein R^{x17} is selected from a hydrido, alkyl, aryl, cycloalkyl, heteroaryl or heterocyclyl group; and wherein R^{x18} is selected from an alkyl, aryl, cycloalkyl, heteroaryl or heterocyclyl group.

The term “thioureido” is defined as a sulfur radical of the formula 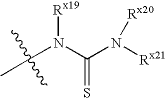
wherein each of R^{x19} and R^{x20} is independently selected from hydrido, alkyl, aryl, cycloalkyl, heteroaryl or heterocyclyl group; and R^{x21} is selected from an alkyl, aryl, cycloalkyl, heteroaryl or heterocyclyl group.

The group trityl is a triphenylmethyl group.

The term “ureido” is defined as a nitrogen radical of the formula 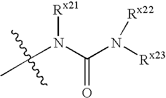
wherein each of R^{x21} and R^{x22} is independently selected from hydrido, alkyl, aryl, cycloalkyl, heteroaryl or heterocyclyl group; and R^{x23} is selected from an alkyl, aryl, cycloalkyl, heteroaryl or heterocyclyl group.

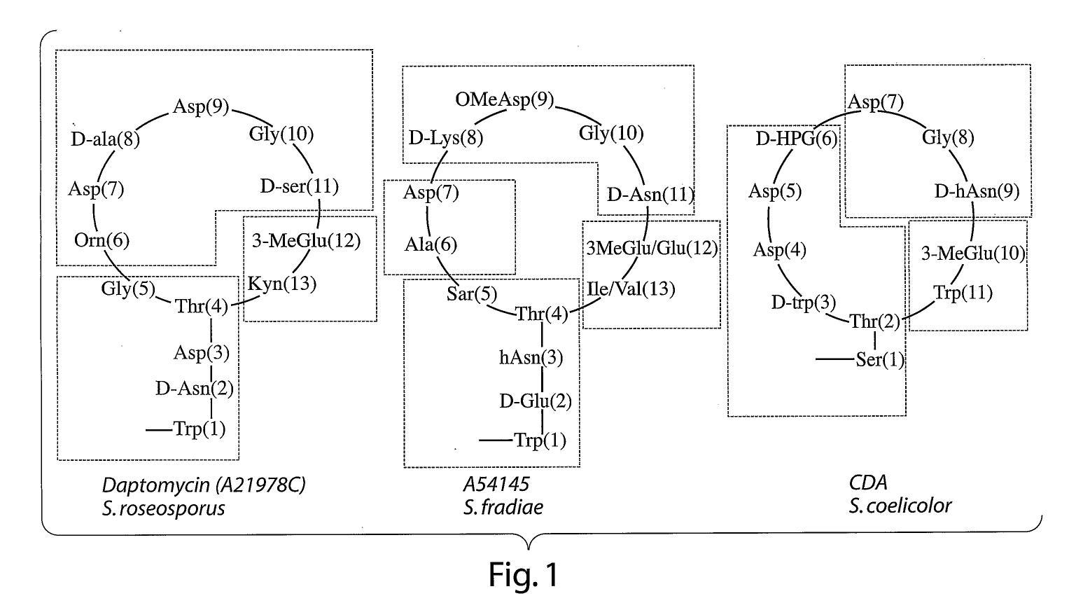

The terms “lptA”, “lptB” “lptC” and “lptD” refer to nucleic acid molecules that encode subunits of the A54145 NRPS. In a preferred embodiment, the nucleic acid molecule is derived from Streptomyces, more preferably the nucleic acid molecule is derived from S. fradiae. The lptA nucleic acid encodes for amino acids 1-5. The lptB nucleic acid encodes for amino acids 6 and 7. The lptC nucleic acid encodes for amino acids 8-11. The lptD nucleic acid encodes for amino acids 12 and 13 (FIG. 1). The terms “lptA”, “lptB” “lptC” and “lptD” also refer to allelic variants of these genes, which may be obtained from other species of Streptomyces or from other S. fradiae strains.

The terms “dptA”, “dptBC” and “dptD” refer to nucleic acid molecules that encode subunits of the daptomycin NRPS. In a preferred embodiment, the nucleic acid molecule is derived from Streptomyces, more preferably the nucleic acid molecule is derived from S. roseosporus. The dptA nucleic acid encodes amino acids 1-5. The dptBC nucleic acid encodes amino acids 6-11. The dptD nucleic acid encodes amino acids 12-13 (FIG. 1). The terms “dptA”, “dptBC” and “dptD” also refer to allelic variants of these genes, which may be obtained from other species of Streptomyces or from other S. roseosporus strains.

The salts of the compounds of the invention include acid addition salts and base addition salts. In a preferred embodiment, the salt is a pharmaceutically acceptable salt of the compound of Formula I or the compound of any of Formula F1-F22. The term “pharmaceutically acceptable salts” embraces salts commonly used to form alkali metal salts and to form addition salts of free acids or free bases. The nature of the salt is not critical, provided that it is pharmaceutically-acceptable. Suitable pharmaceutically acceptable acid addition salts of the compounds of the invention may be prepared from an inorganic acid or an organic acid. Examples of such inorganic acids include, without limitation, hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which include, without limitation, formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Suitable pharmaceutically-acceptable base addition salts of compounds of the invention include, but are not limited to, metallic salts made from aluminum, calcium, lithium, magnesium, potassium, sodium and zinc or organic salts made from N,N′-dibenzylethylenediamine, chloroprocaine, choline, diethanolamine, ethylenediamine, N-methylglucamine, lysine and procaine. All of these salts may be prepared by conventional means from the corresponding compound of the invention by treating, for example, the compound of the invention with the appropriate acid or base.

The compounds of the invention can possess one or more asymmetric carbon atoms and are thus capable of existing in the form of optical isomers as well as in the form of racemic or non-racemic mixtures thereof. The compounds of the invention can be utilized in the present invention as a single isomer or as a mixture of stereochemical isomeric forms. Diastereoisomers, i.e., nonsuperimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from the optically active salts. An alternative process for separation of optical isomers includes the use of a chiral chromatography column optimally chosen to maximize the separation of the enantiomers. Still another method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of the invention can likewise be obtained by utilizing optically active starting materials. These isomers may be in the form of a free acid, a free base, an ester or a salt.

The invention also embraces isolated compounds, preferably compounds of Formula I or compounds of any of Formulas F1-F22. An isolated compound refers to a compound, preferably a compound of Formula I or a compound of any of Formulas F1-F22, which represents at least about 1%, preferably at least about 10%, more preferably at least about 20%, even more preferably at least about 50%, yet more preferably at least about 80%, yet even more preferably at least about 90% and most preferably at least about 99% of the compound present in the mixture. In one embodiment of the invention the compound, preferably a compound of Formula I or a compound of any of Formulas F1-F22, is present in at least about 80% to about 90% of the composition. In another embodiment the compound, preferably a compound of Formula I or a compound of any of Formulas F1-F22, is present in at least 90% of the composition. In another embodiment the compound, preferably a compound of Formula I or compound of any of Formulas F1-F22, is present in greater than 90% of the composition.

The percentation of the compound, preferably a compound of Formula I or a compound of any of Formulas F1-F22, may be measured by any means including nuclear magnetic resonance (NMR), gas chromatography/mass spectroscopy (GC/MS), liquid chromatography/mass spectroscopy (LC/MS) or microbiological assays. A preferred means for measuring the purity of the compound is by analytical high pressure liquid chromatography (HPLC) or LC/MS.

In one embodiment of the invention, the compound, a pharmaceutically acceptable salt thereof or a pharmaceutical composition comprising the compound exhibits a detectable (i.e. statistically significant) antimicrobial activity when tested in conventional biological assays such as those described herein.

Depsipeptide Compounds

In one aspect, the invention provides compounds of Formula I 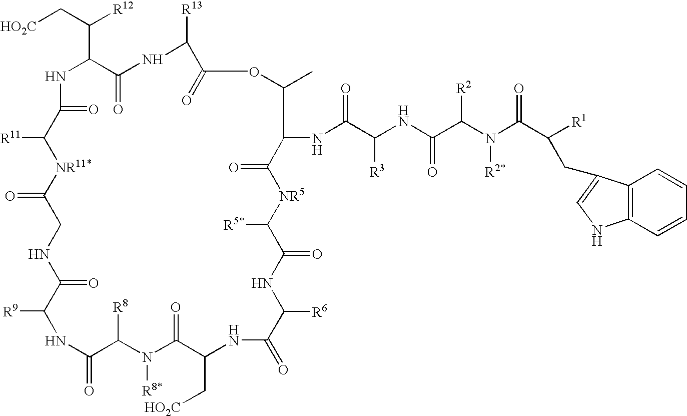
and salts thereof.

The group R² of Formula I is an amino acid side chain, 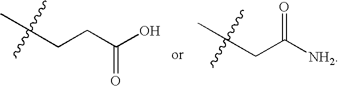
In one embodiment of the invention the amino acid side chain is 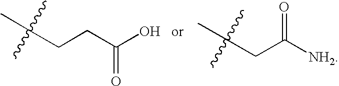
In another embodiment of the invention, the amino acid side chain is derived from a D-amino acid. In another embodiment of the invention, the amino acid side chain is 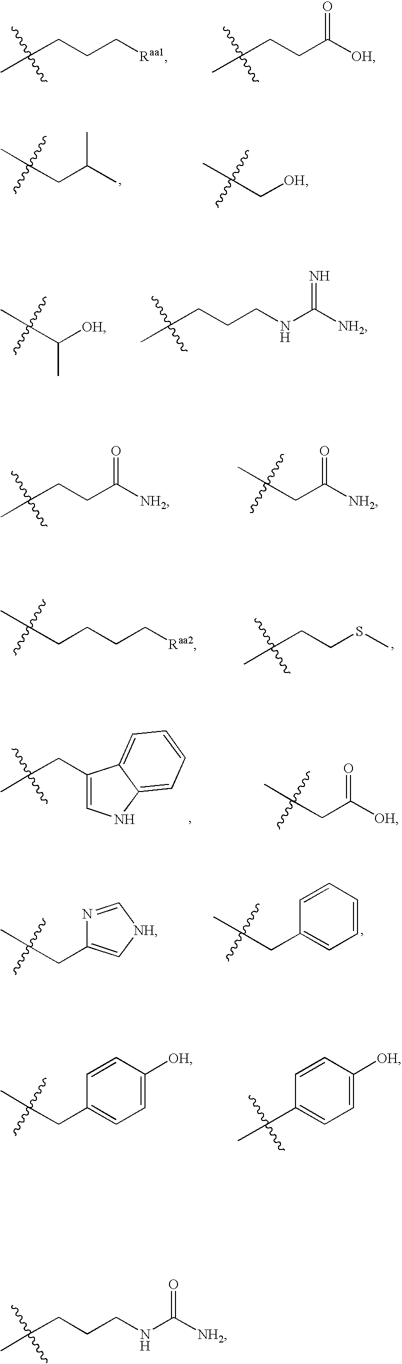 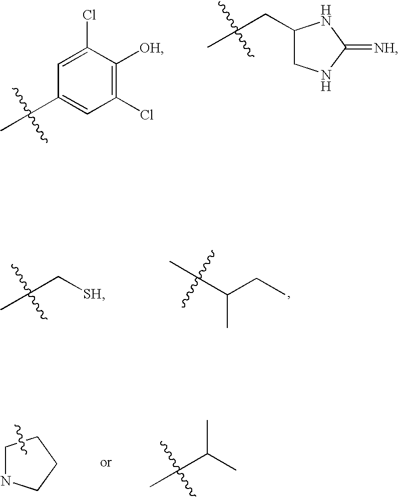
wherein each of R^{aa1} and R^{aa2} is independently amino, monosubstituted amino, disubstituted amino, acylamino, ureido, guanidino, carbamoyl, sulfonamino, thioacylamino, thioureido, iminoamino, or phosphonamino.

Substituent R²* is H. Alternatively, R² and R²* together with the atoms to which they are attached, form a five or six-member heterocyclic ring. In one embodiment of Formula I, R² and R²* together with the atoms to which they are attached, form a pyrrolidine ring.

The group R³ of Formula I is 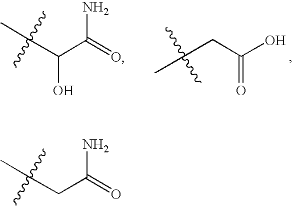
or a non-proteinogenic amino acid side chain. In one embodiment of the invention the group R³ of Formula I is 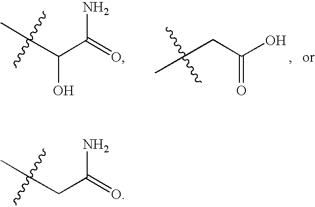
In another embodiment of the invention, the non-proteinogenic amino acid is

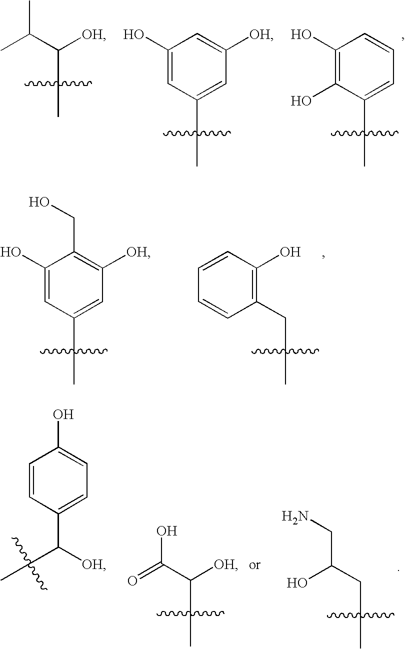

Substituent R⁵ of Formula I is H or methyl and substituent R⁵ of Formula I is H or an amino acid side chain derived from an N-methylamino acid. In one embodiment of the invention, R⁵* is methyl, 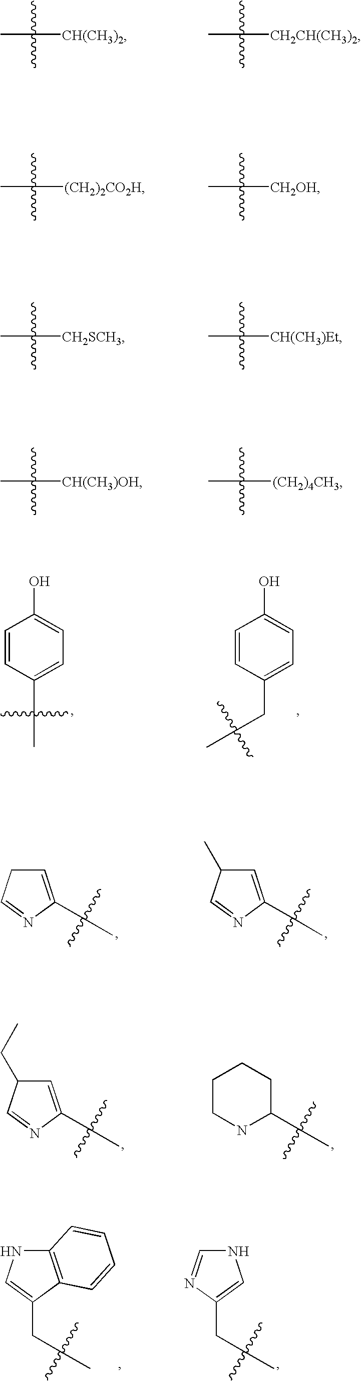 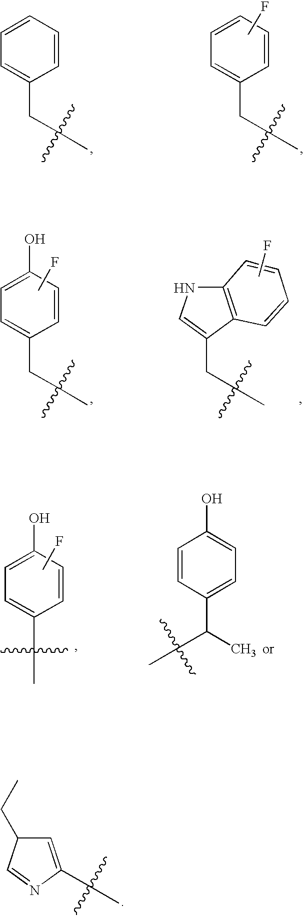
Alternatively, R⁵ and R⁵* together with the atoms to which they are attached, form a five or six-member heterocyclic ring. In one embodiment of Formula I, R⁵ and R⁵* together with the atoms to which they are attached, form a piperidine or a pyrrolidine ring.

Group R⁶ of Formula I is methyl or

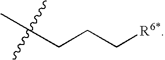

Substituent R⁸ of Formula I is an amino acid side chain, hydrogen, methyl, 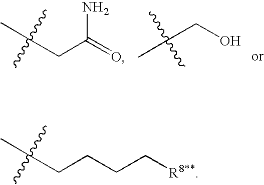
In one embodiment of the invention, substituent R⁸ of Formula I is hydrogen, methyl, 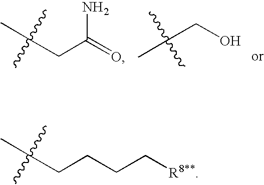
In another embodiment of the invention, the amino acid side chain is derived from a D-amino acid. In another embodiment of the invention substituent R⁸ is the amino acid side chain derived from glycine, D-alanine, D-asparagine, D-serine or D-lysine. In another embodiment of the invention, the amino acid side chain is 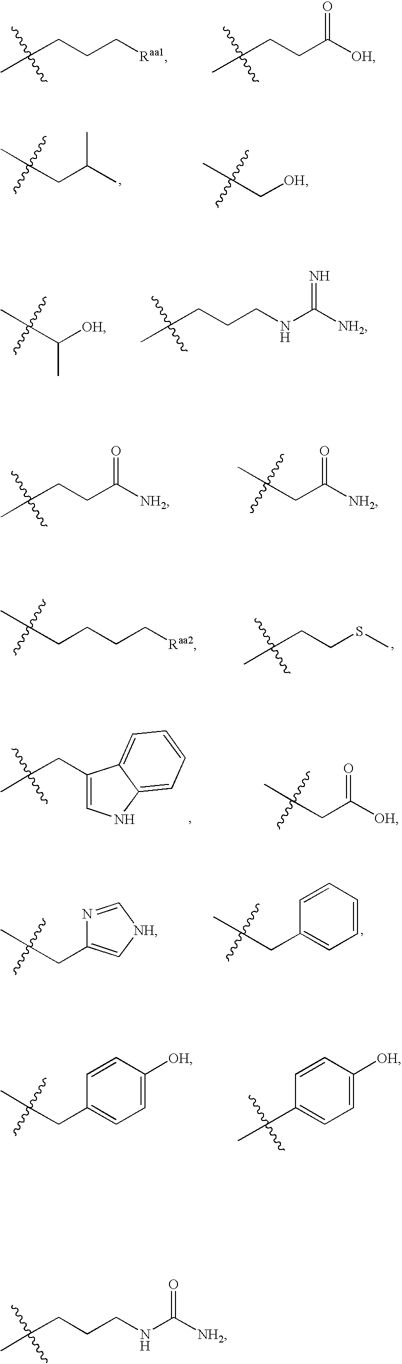 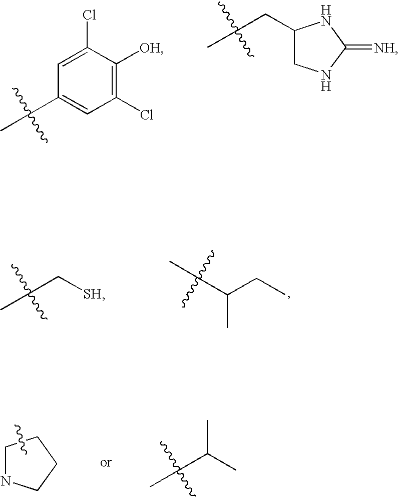
wherein each of R^{aa1} and R^{aa2} is independently amino, monosubstituted amino, disubstituted amino, acylamino, ureido, guanidino, carbamoyl, sulfonamino, thioacylamino, thioureido, iminoamino, or phosphonamino.

Substituent R⁸* of Formula I is H. Alternatively, R⁸ and R⁸* together with the atoms to which they are attached, form a five or six-member heterocyclic ring. In one embodiment of Formula I, R⁸ and R⁸* together with the atoms to which they are attached, form a pyrrolidine ring.

Group R⁹ of Formula I is 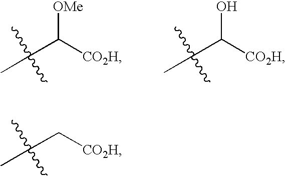
or an amino acid side chain substituted with at least one carboxylic acid. In one embodiment of the invention group R⁹ of Formula I is 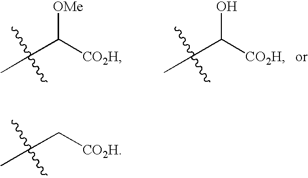
In another embodiment of the invention, the amino acid side chain is

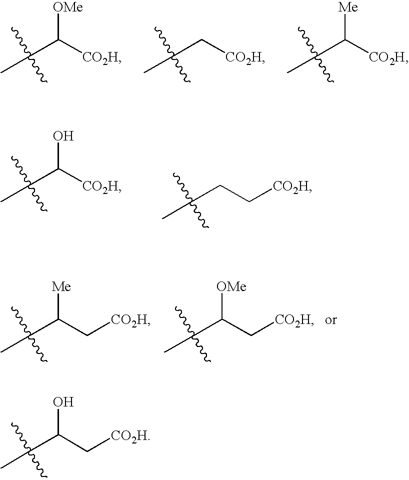

Substituent R¹¹ of Formula I is an amino acid side chain, methyl, 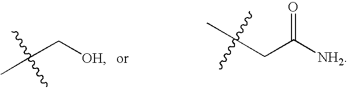
In one embodiment of the invention substituent R¹¹ of Formula I is methyl, 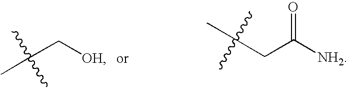
In one embodiment of the invention, the amino acid side chain is derived from a D-amino acid. In another embodiment of the invention R¹¹ of Formula I is an amino acid side chain derived from D-alanine, D-serine, or D-asparagine. In another embodiment of the invention, the amino acid side chain is 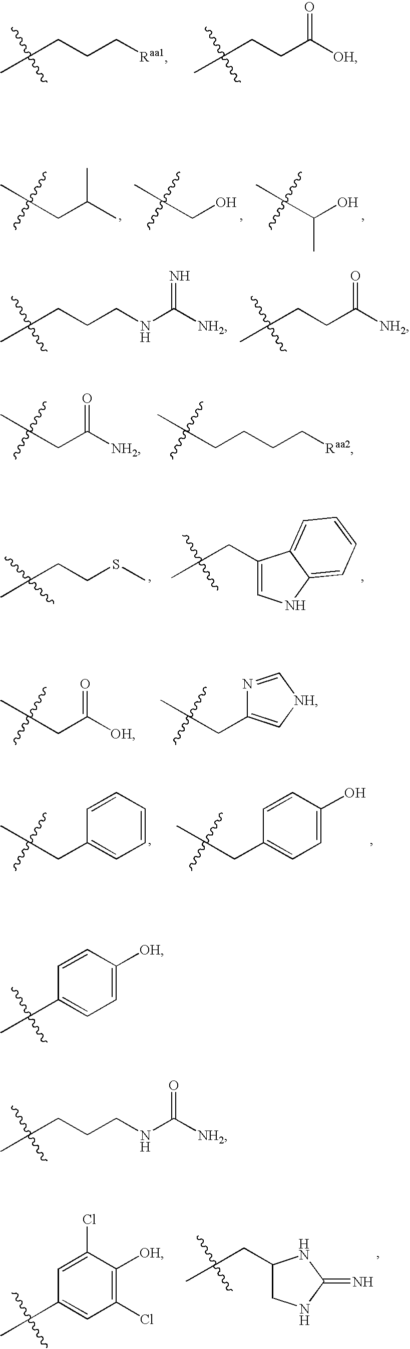 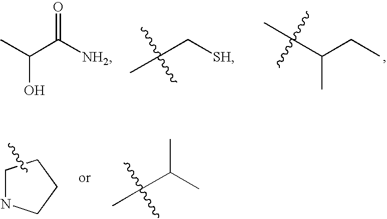
wherein each of R^{aa1} and R^{aa2} is independently amino, monosubstituted amino, disubstituted amino, acylamino, ureido, guanidino, carbamoyl, sulfonamino, thioacylamino, thioureido, iminoamino, or phosphonamino.

Substituent R¹¹* is H. Alternatively, R¹¹ and R¹¹* together with the atoms to which they are attached, form a five or six-member heterocyclic ring. In one embodiment of Formula I, R¹¹ and R¹¹* together with the atoms to which they are attached, form a pyrrolidine ring.

Group R¹² of Formula I is H or CH₃.

Substituent R¹³ of Formula I is CH(CH₃)₂, CH(CH₂CH₁₃)CH₃,

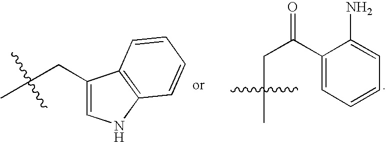

In one embodiment of the invention, R¹³ is CH(CH₂CH₃)CH₃ or

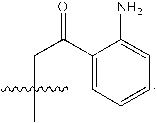

Each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino. In one embodiment of the invention R¹ is amino, NH-amino protecting group, or acylamino. In another embodiment of the invention R¹ is amino. In another embodiment of the invention, R¹ is NH-amino protecting group. In another embodiment of the invention R¹ is acylamino. In another embodiment of the invention R¹ is alkanoylamino. In yet another embodiment of the invention R¹ is C₁₀-C₁₃ alkanoylamino. In still another embodiment of the invention, R¹ is

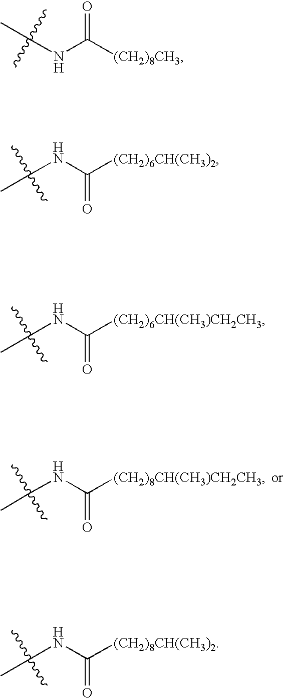

In another embodiment of the invention each of R⁶* and R⁸** is independently amino, or NH-amino protecting group. In another embodiment of the invention each of R⁶* and R⁸** is independently amino. In yet another embodiment of the invention each of R⁶* and R⁸** is independently NH-amino protecting group.

Table I provides exemplary compounds of Formula I.

TABLE I
Compounds of Formula I

| # | Compound |
|---|---|
| C1 | 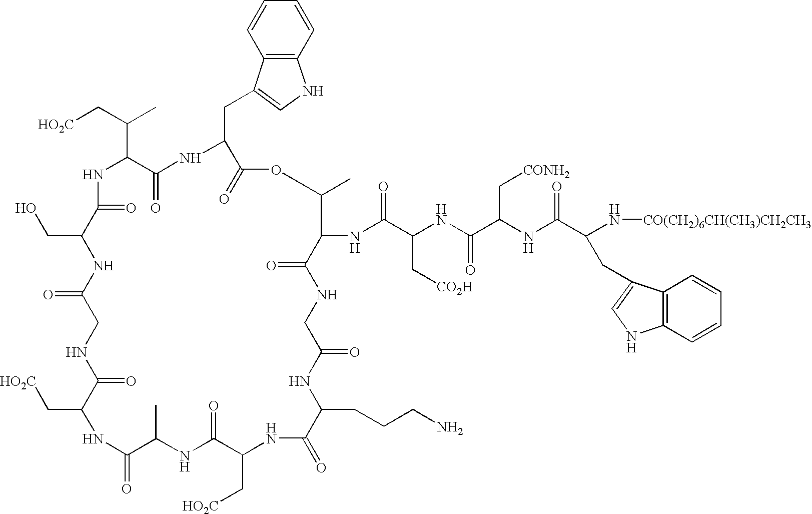 |
| C2 | 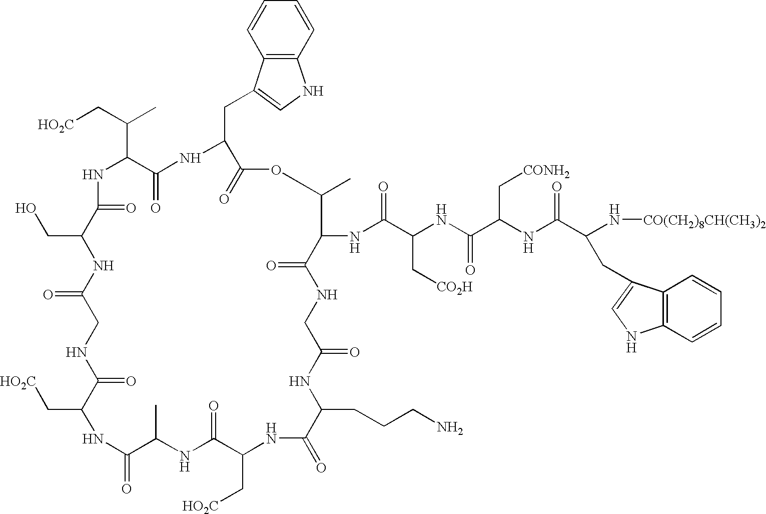 |
| C3 | 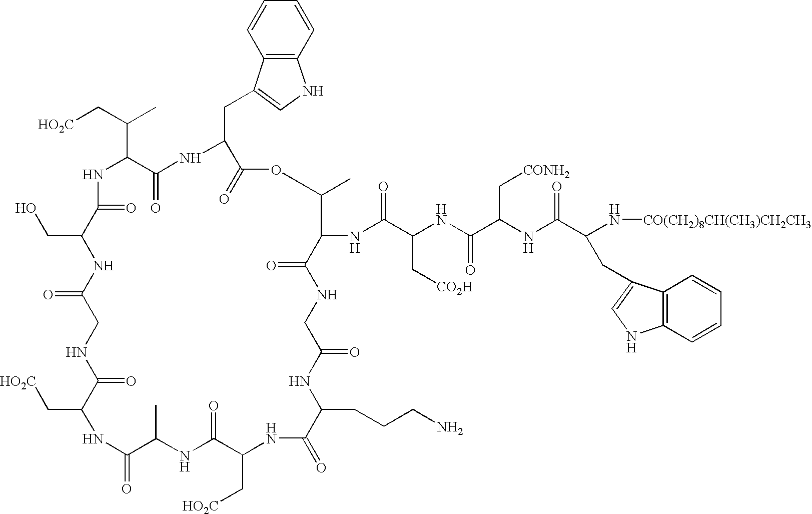 |
| C4 | 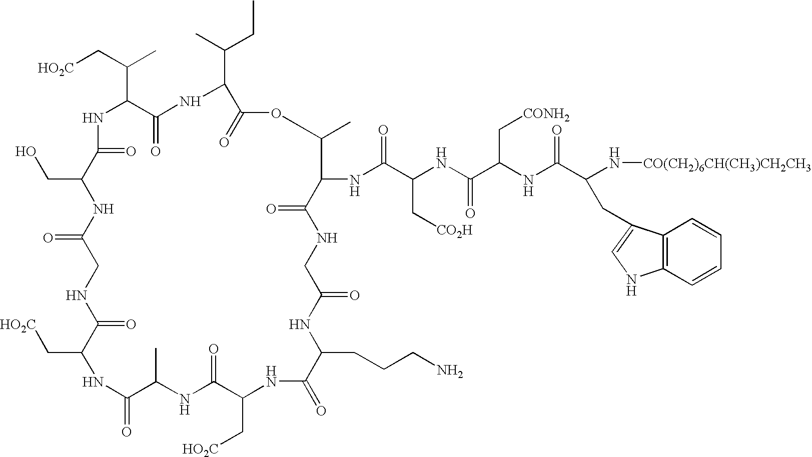 |
| C5 | 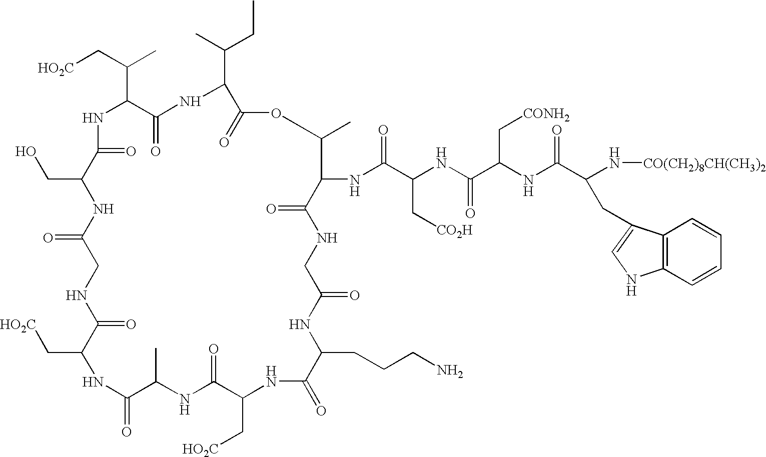 |
| C6 | 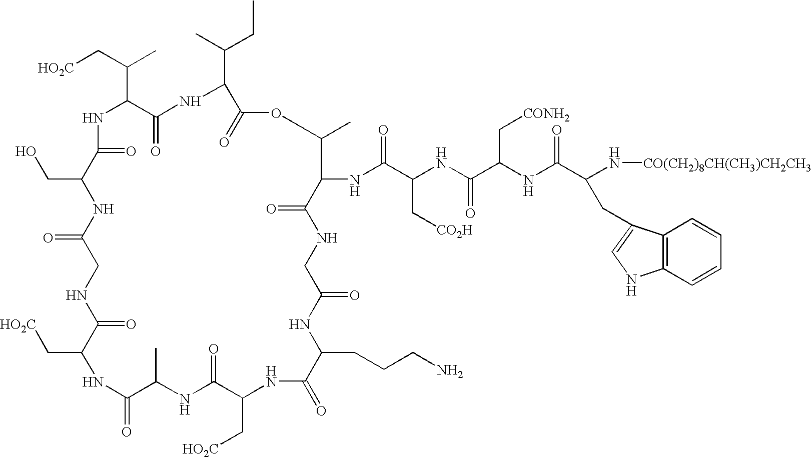 |
| C7 | 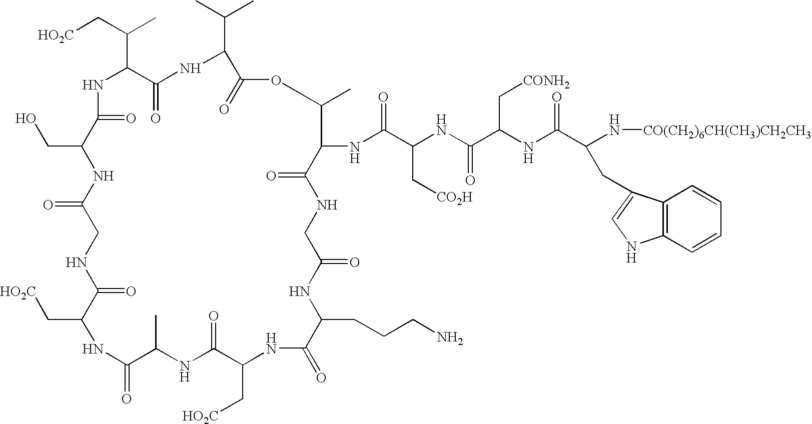 |
| C8 | 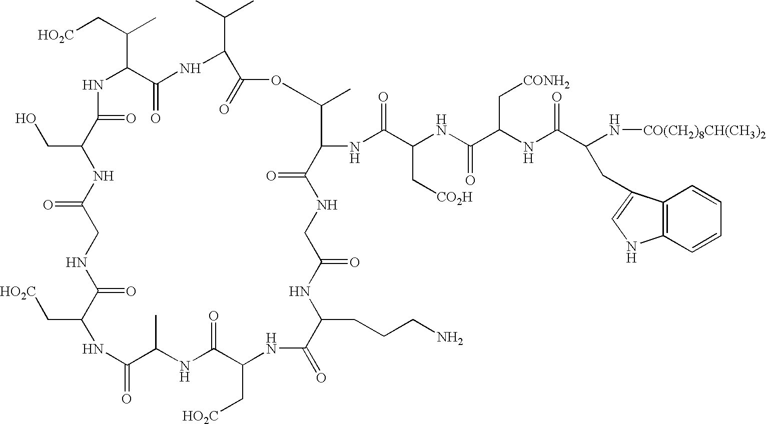 |
| C9 | 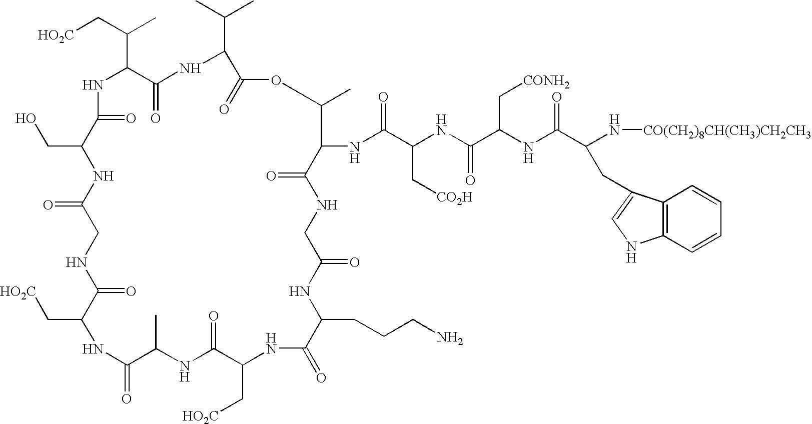 |
| C10 | 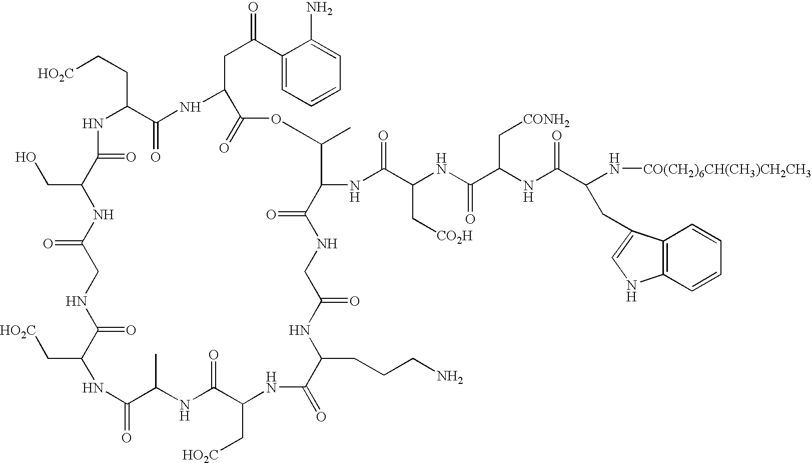 |
| C11 | 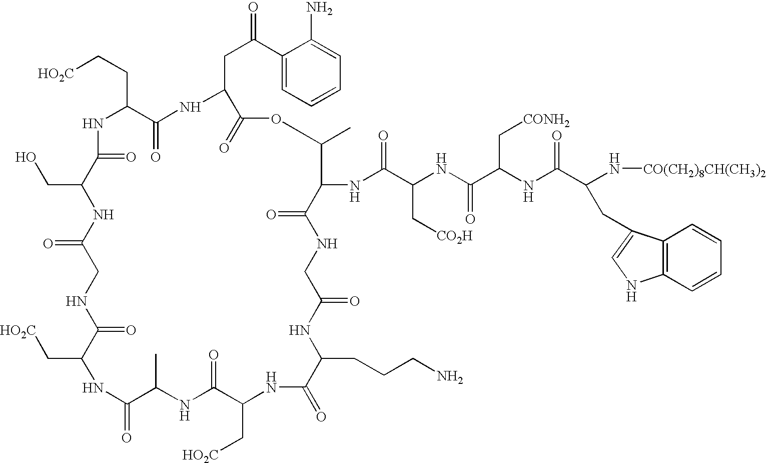 |
| C12 | 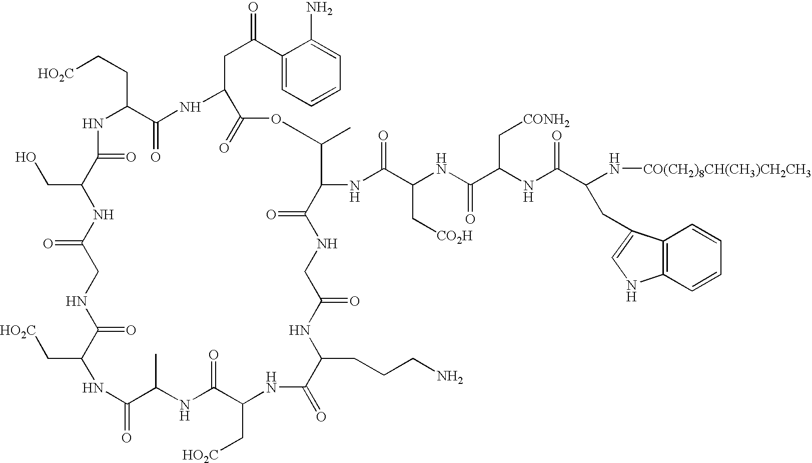 |
| C13 | 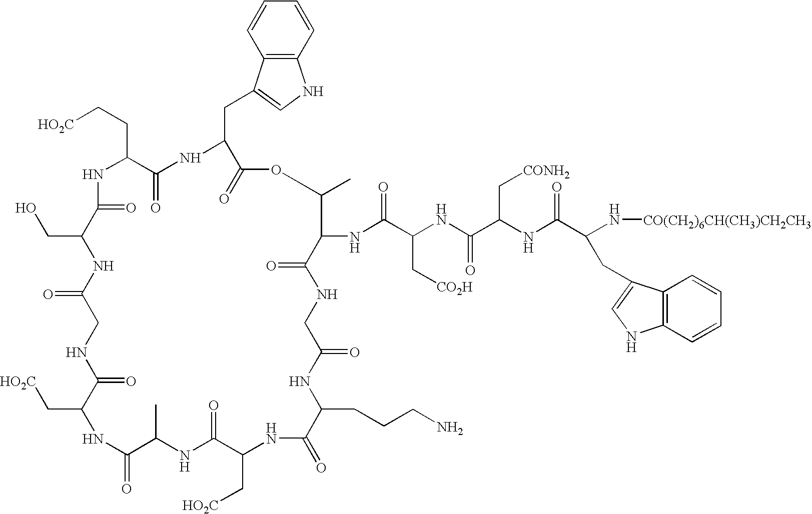 |
| C14 | 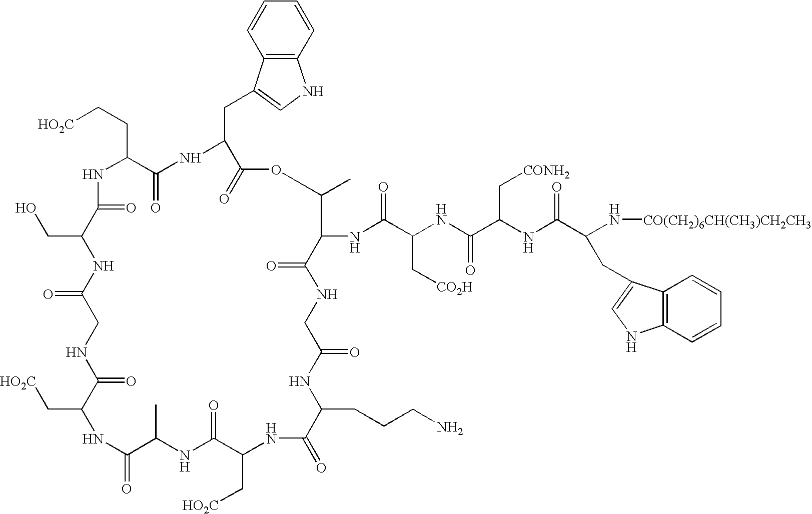 |
| C15 | 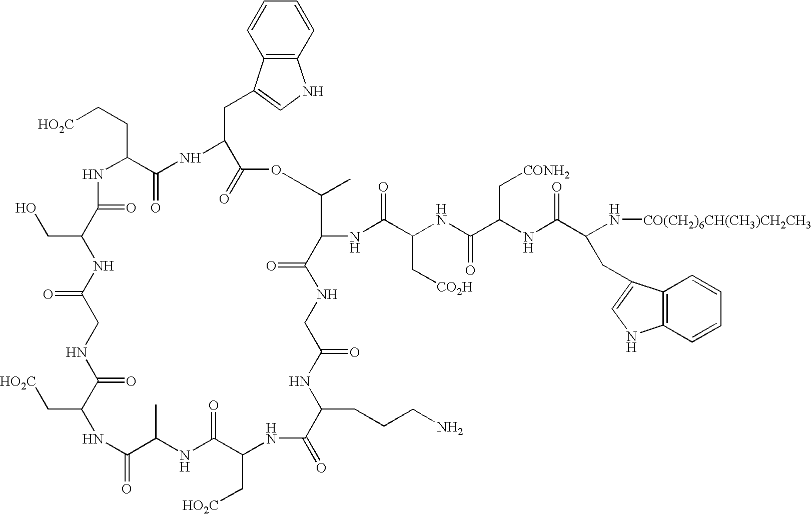 |
| C16 | 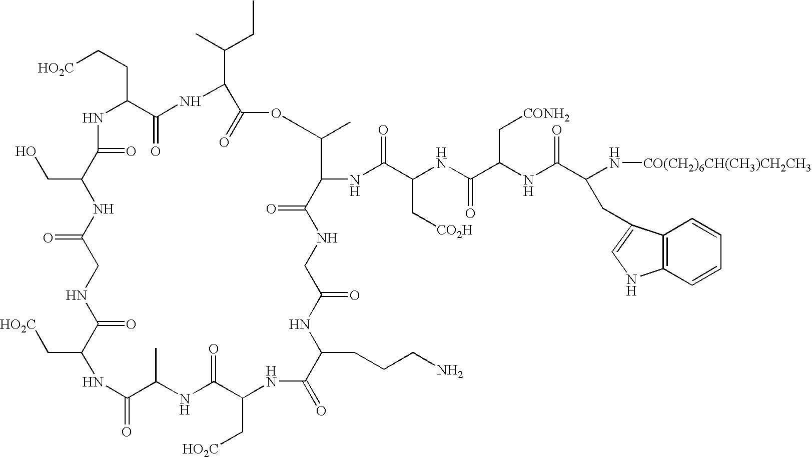 |
| C17 | 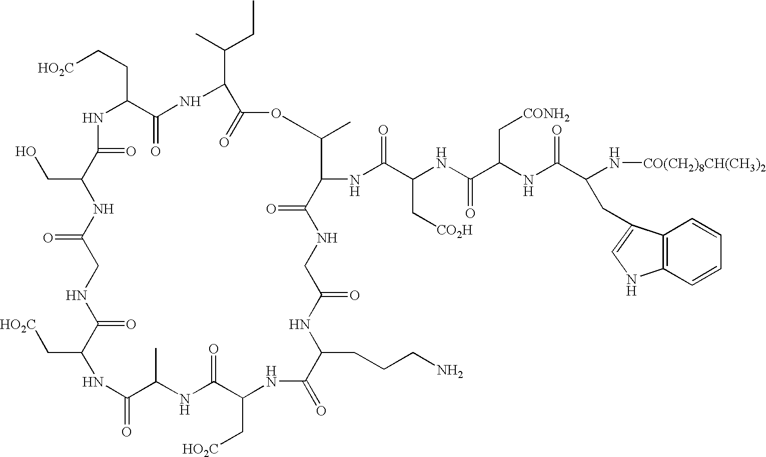 |
| C18 | 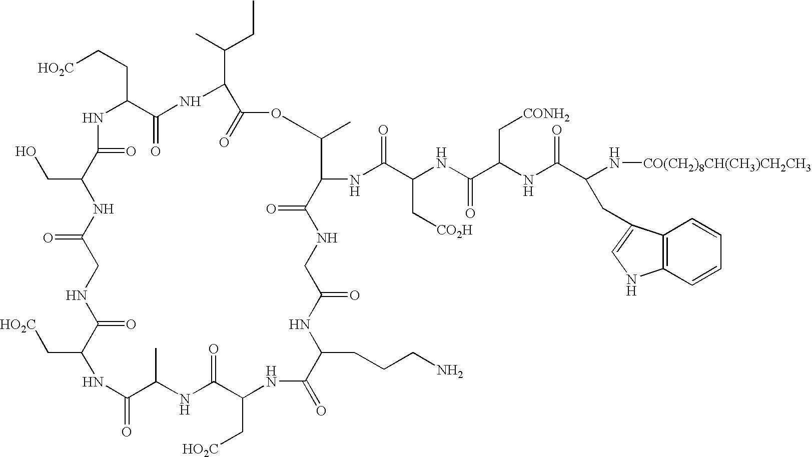 |
| C19 | 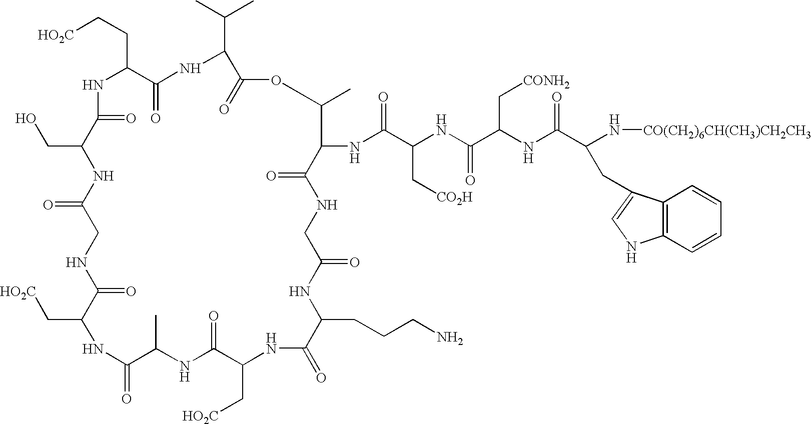 |
| C20 | 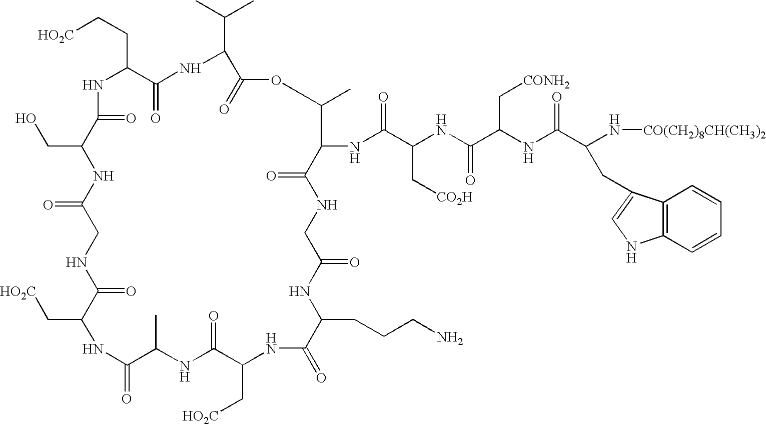 |
| C21 | 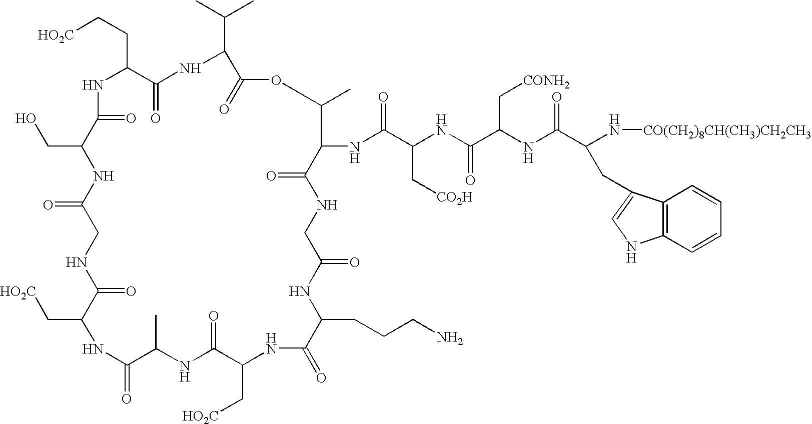 |
| C22 | 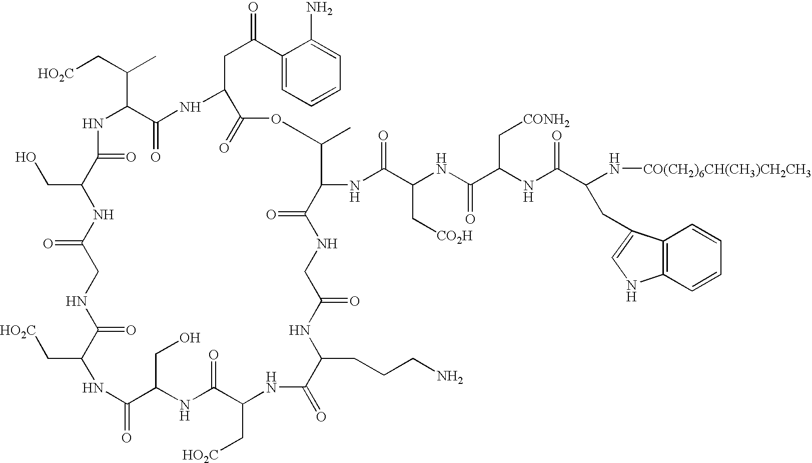 |
| C23 | 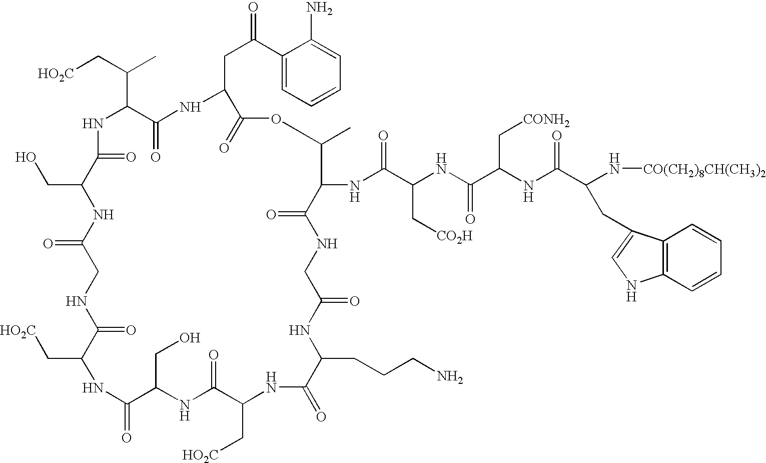 |
| C24 | 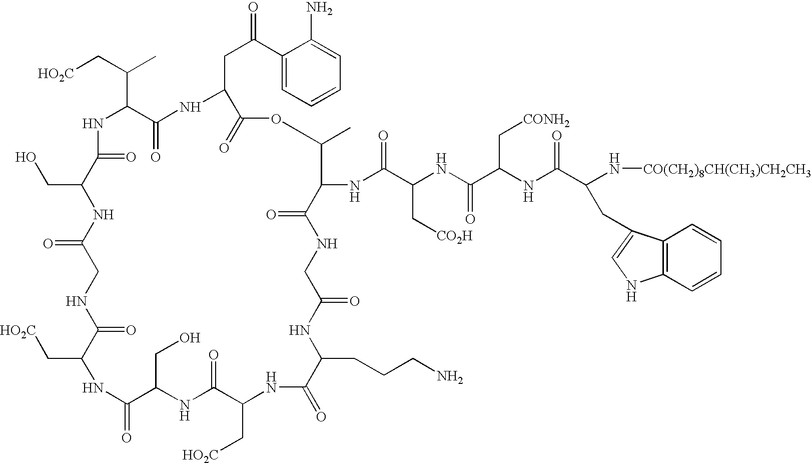 |
| C25 | 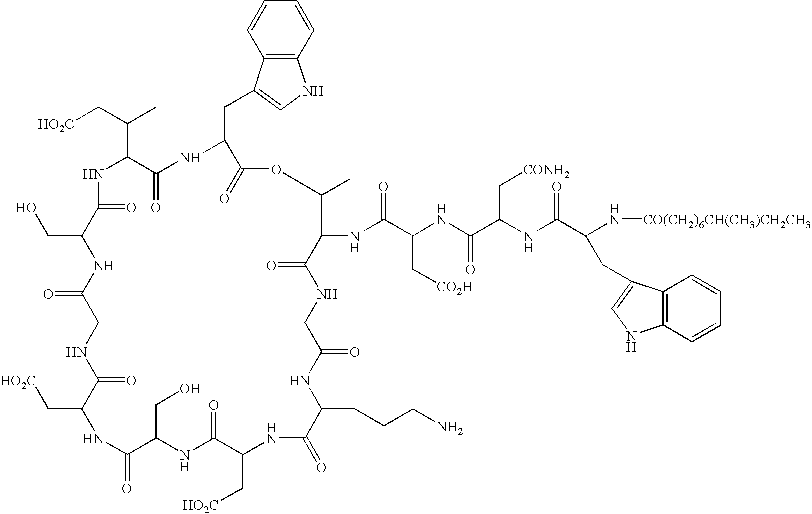 |
| C26 | 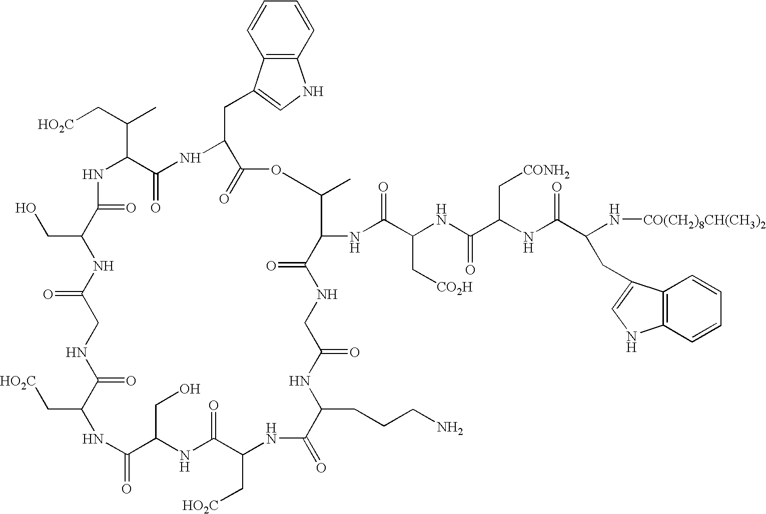 |
| C27 | 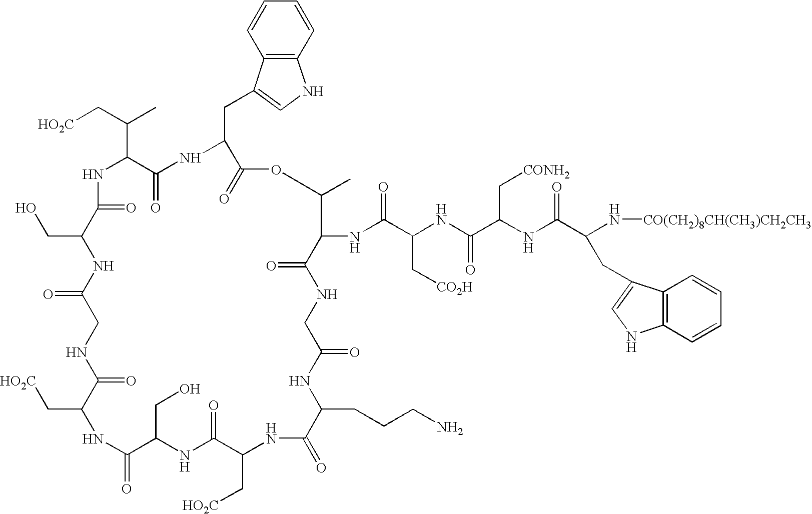 |
| C28 | 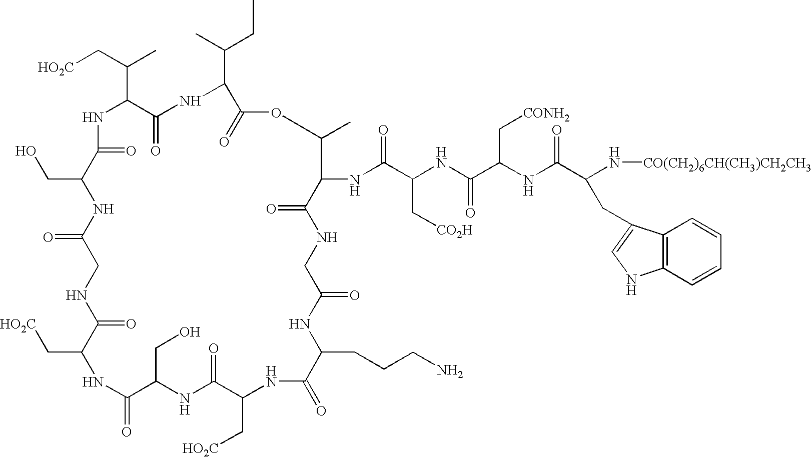 |
| C29 | 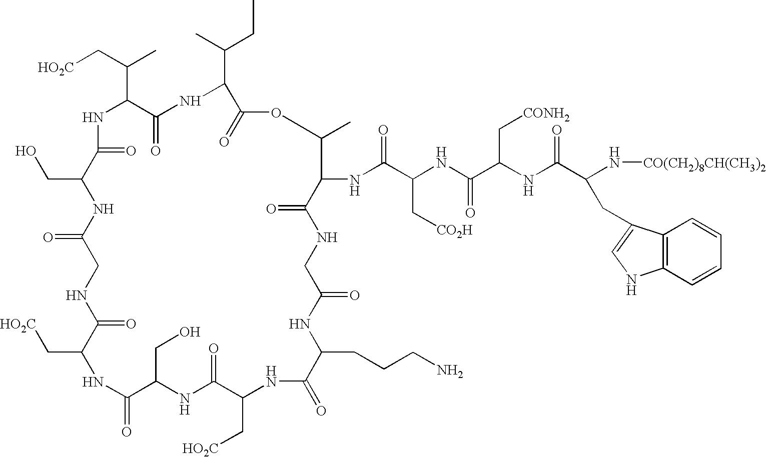 |
| C30 | 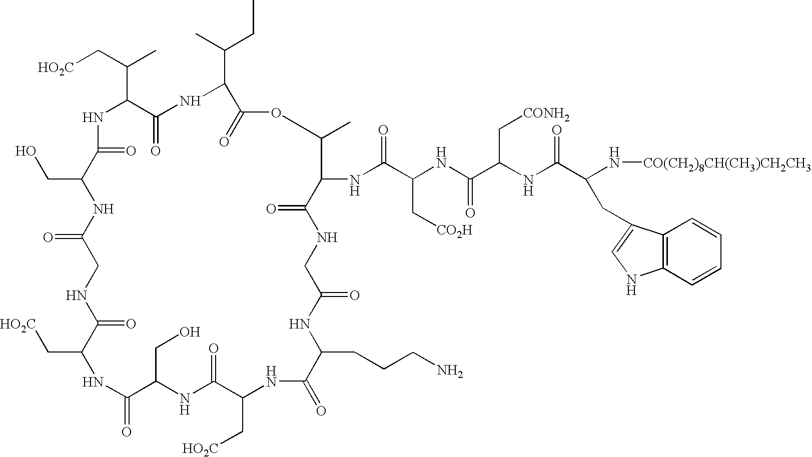 |
| C31 | 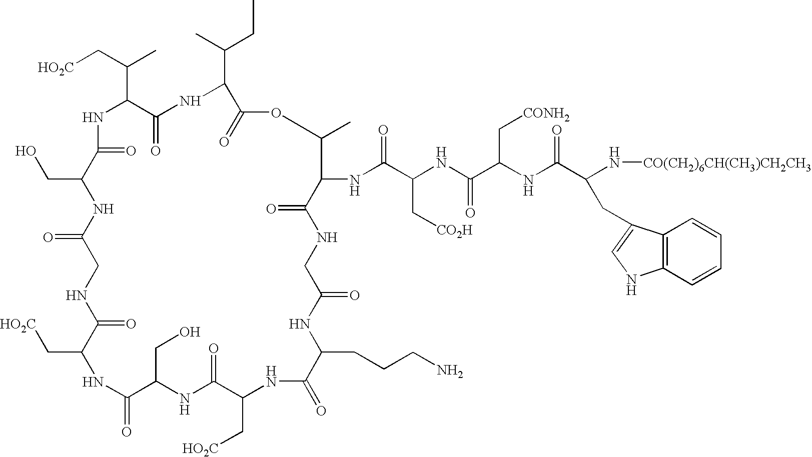 |
| C32 | 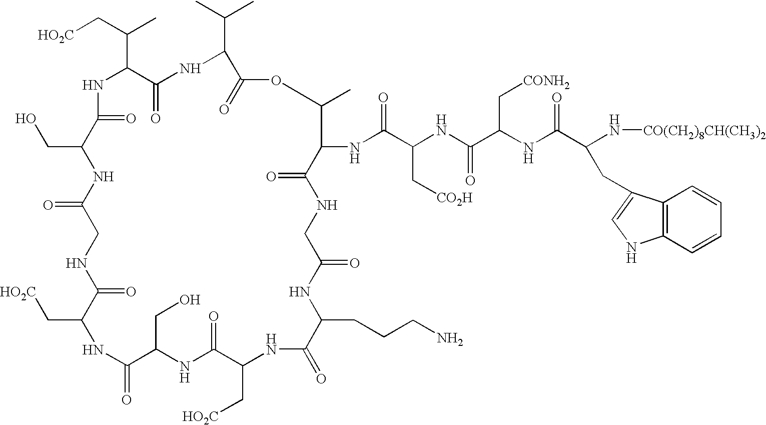 |
| C33 | 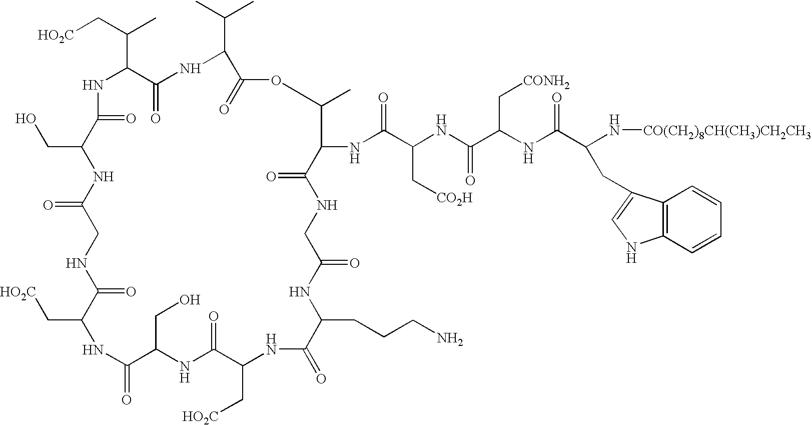 |
| C34 | 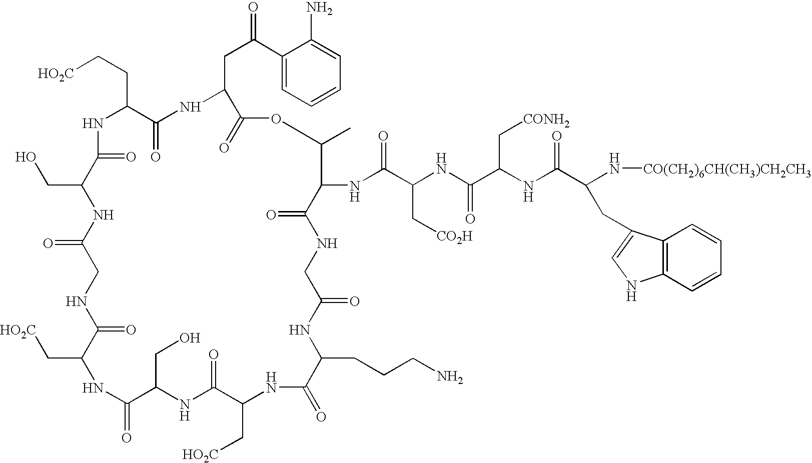 |
| C35 | 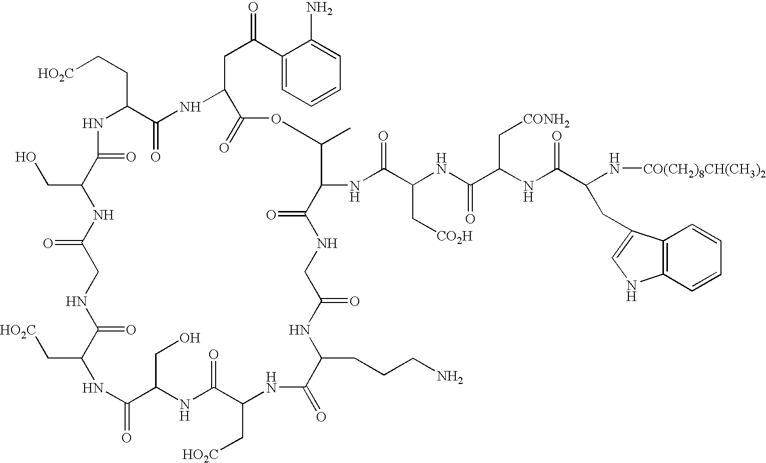 |
| C36 | 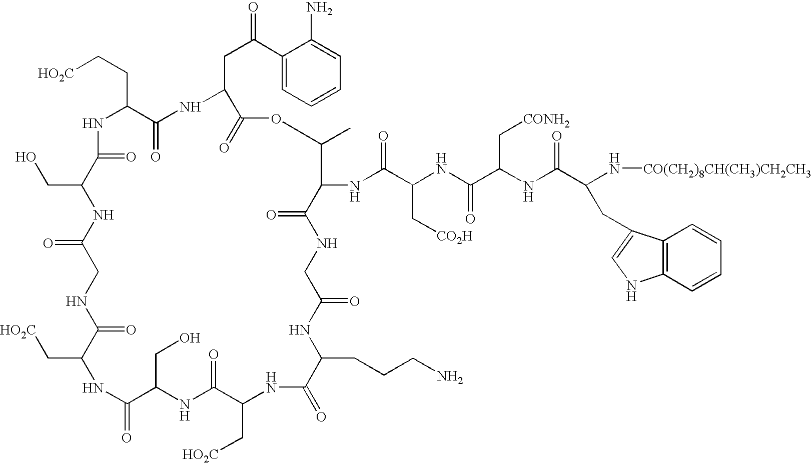 |
| C37 | 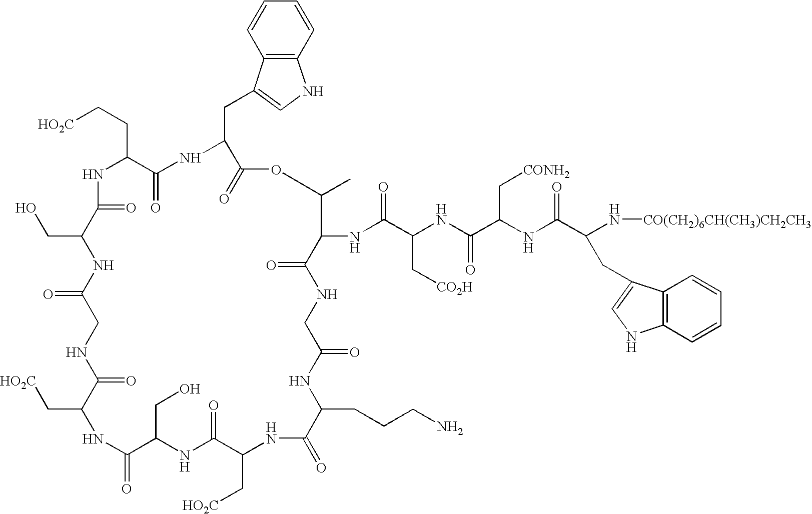 |
| C38 | 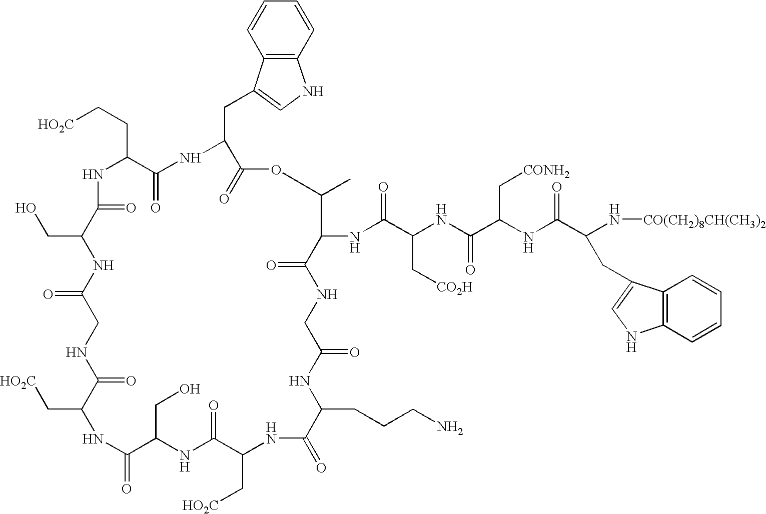 |
| C39 | 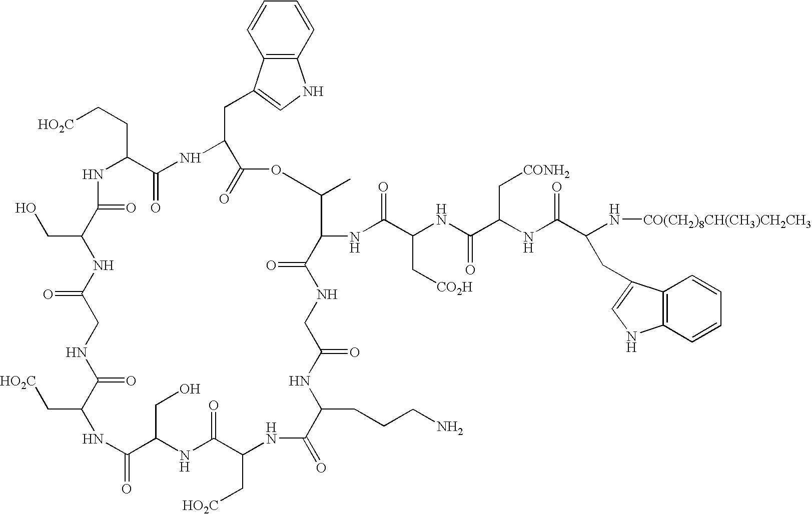 |
| C40 | 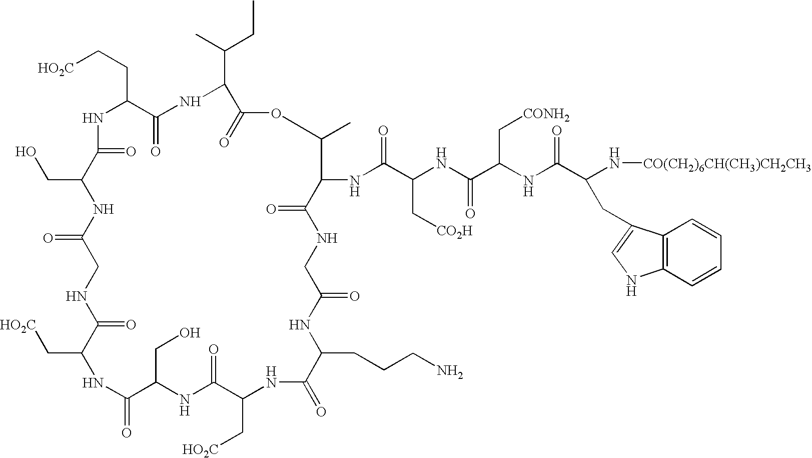 |
| C41 | 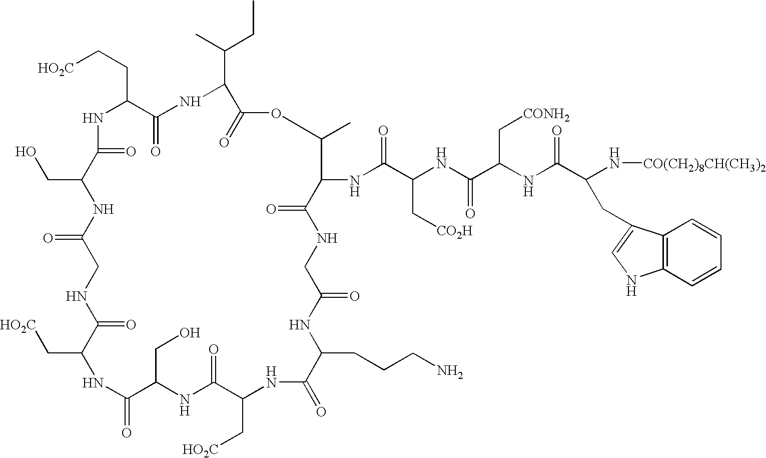 |
| C42 | 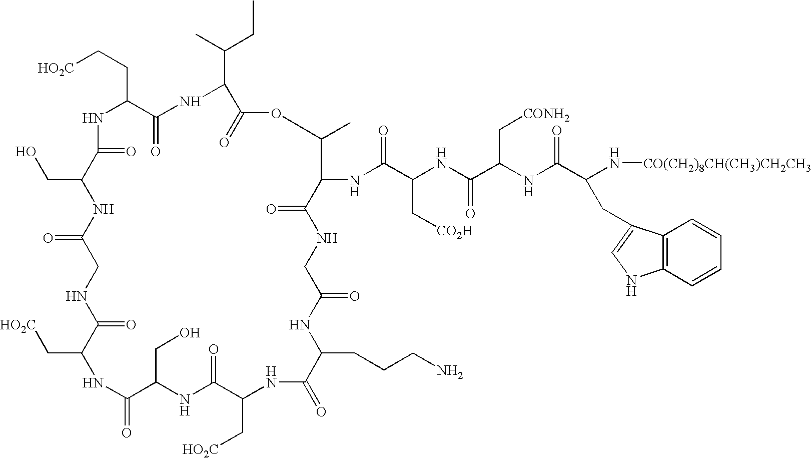 |
| C43 | 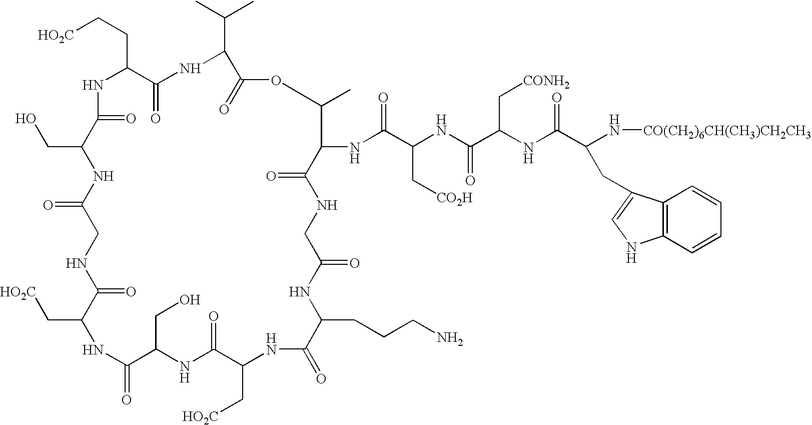 |
| C44 | 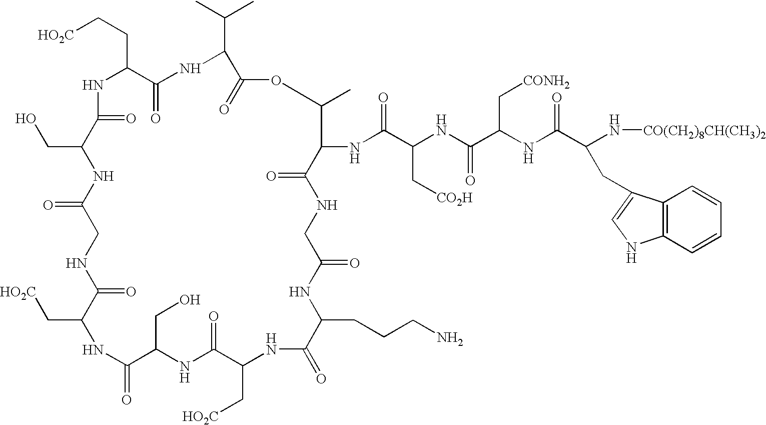 |
| C45 | 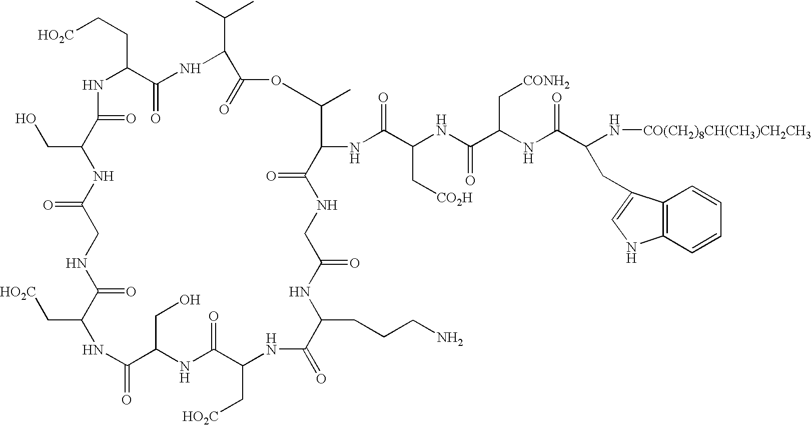 |
| C46 | 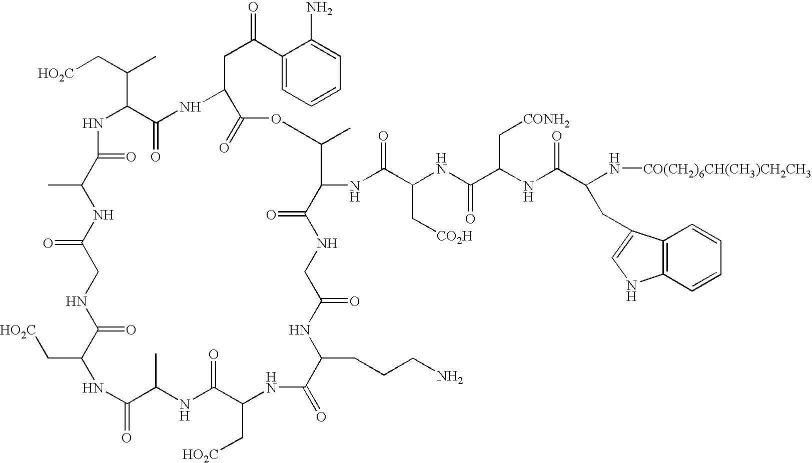 |
| C47 | 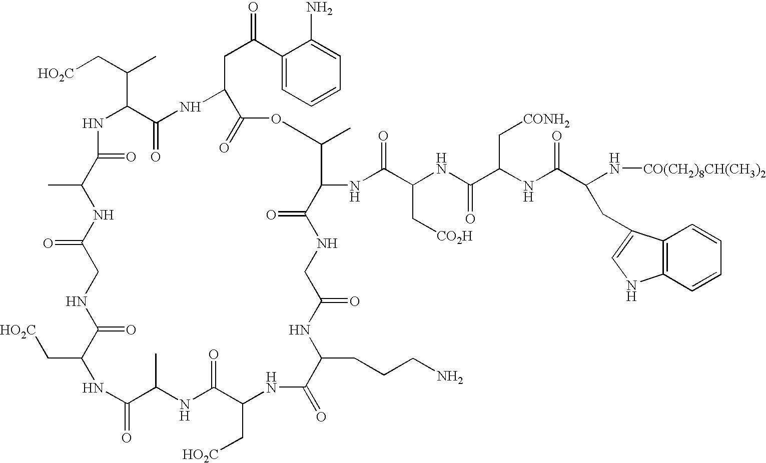 |
| C48 | 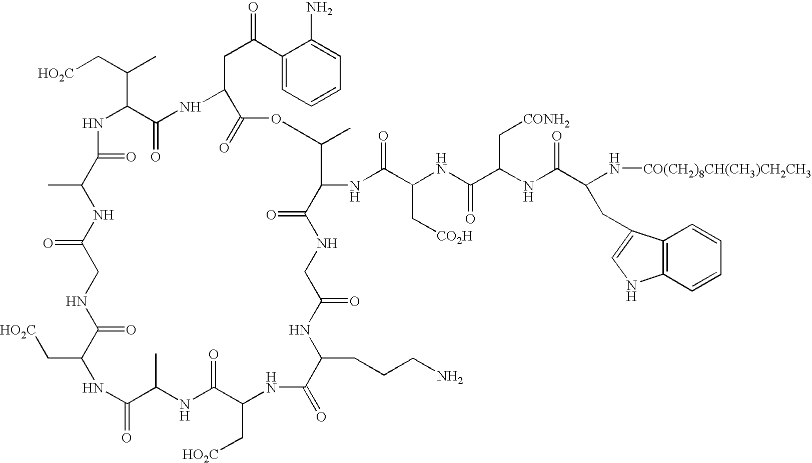 |
| C49 | 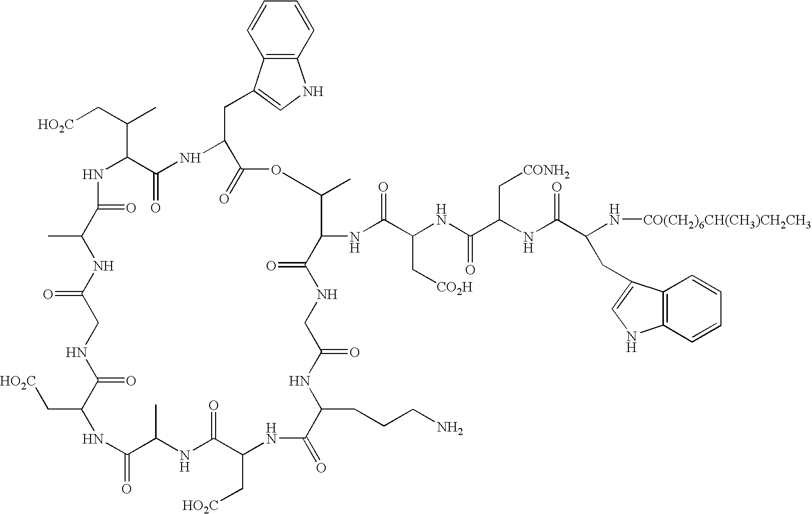 |
| C50 | 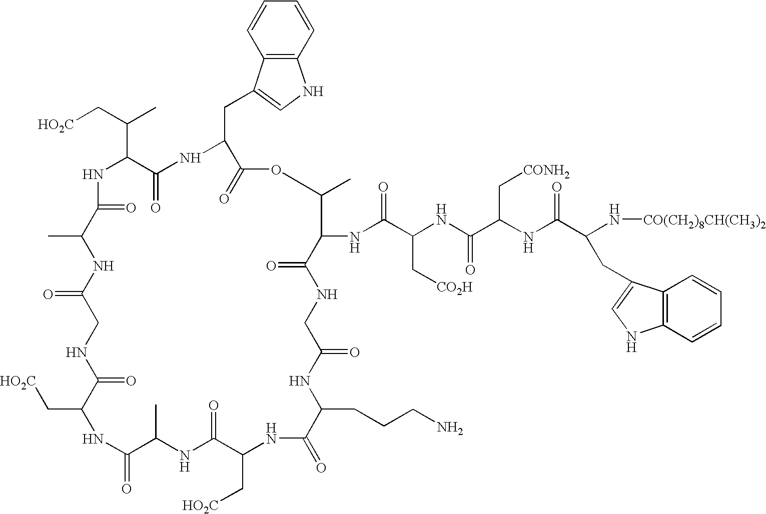 |
| C51 | 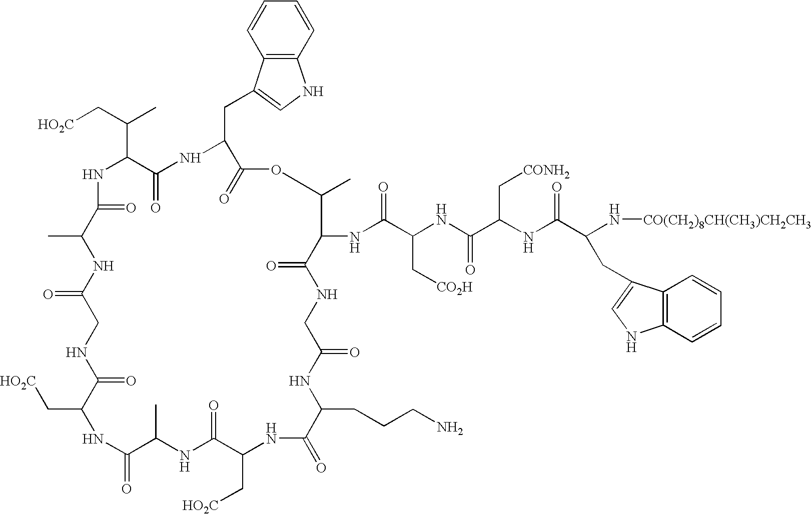 |
| C52 | 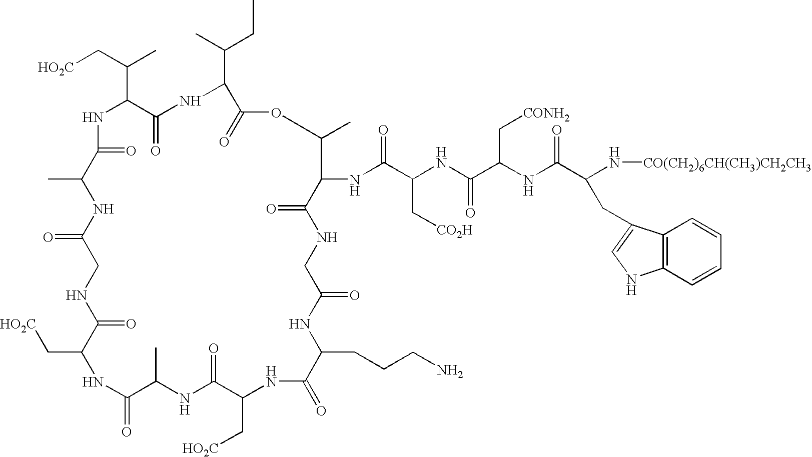 |
| C53 | 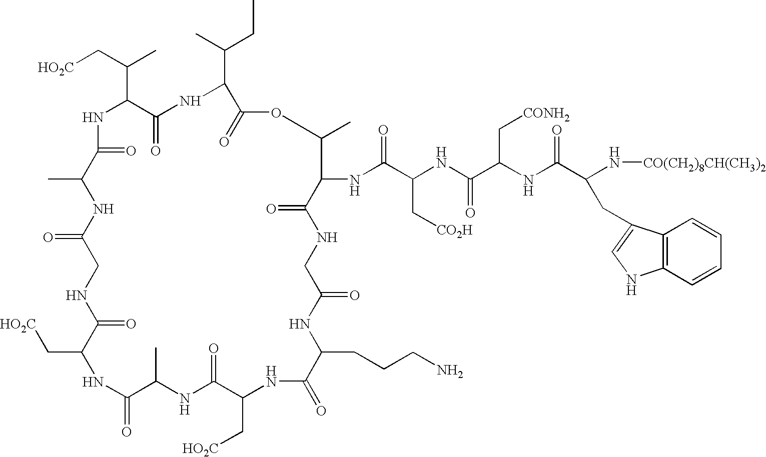 |
| C54 | 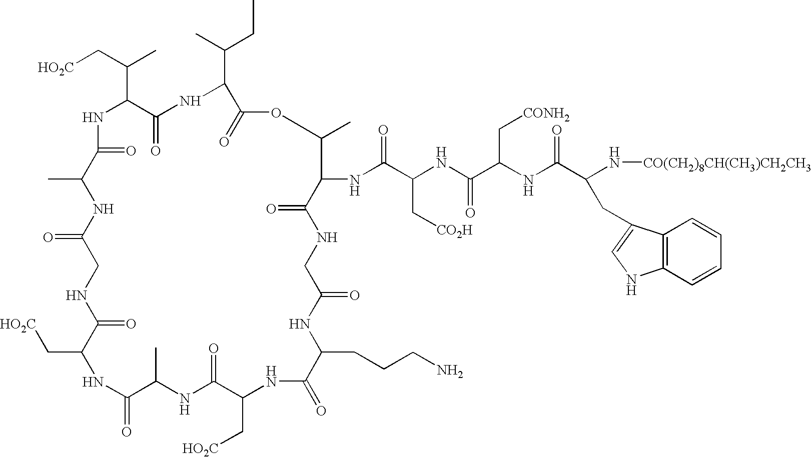 |
| C55 | 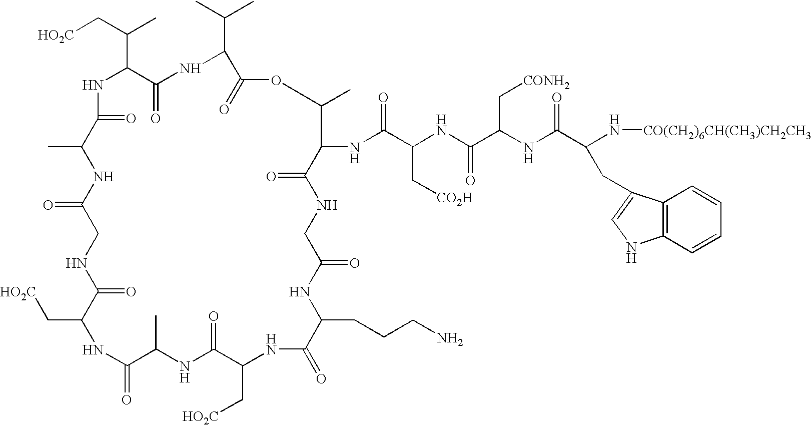 |
| C56 | 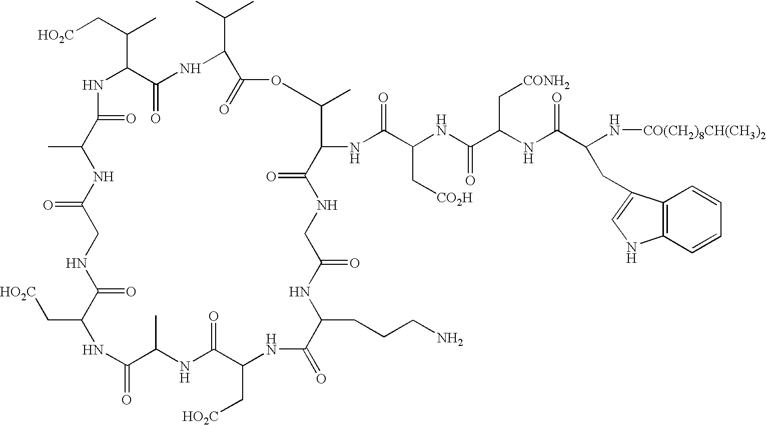 |
| C57 | 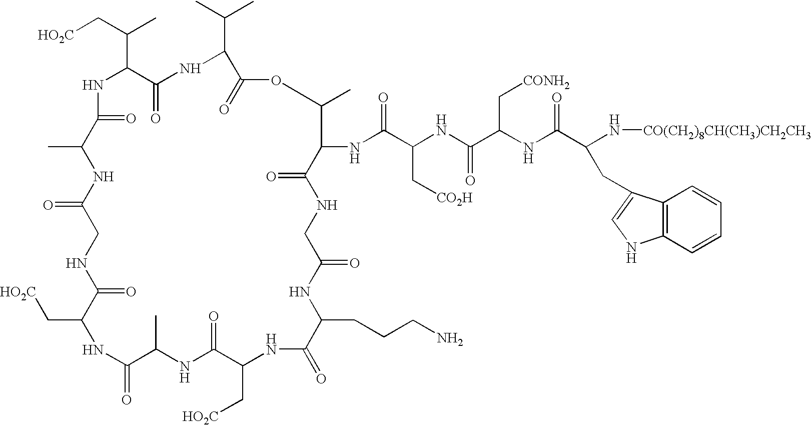 |
| C58 | 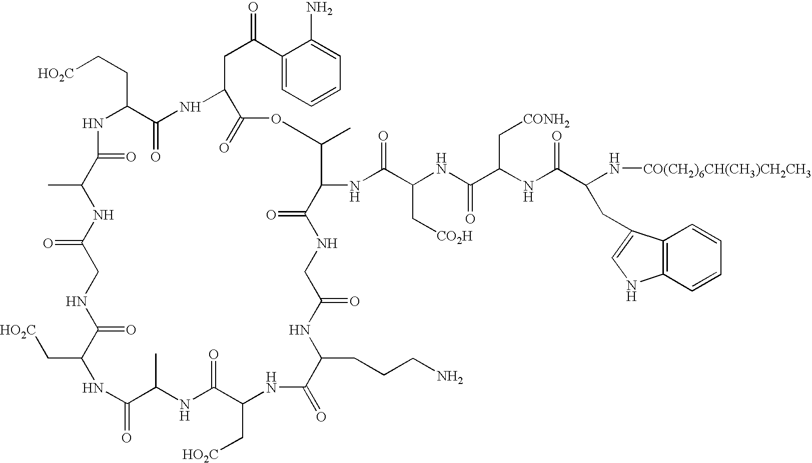 |
| C59 | 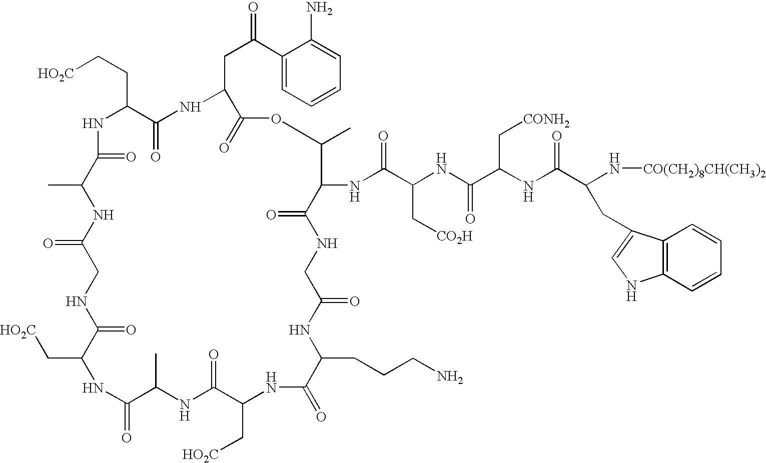 |
| C60 | 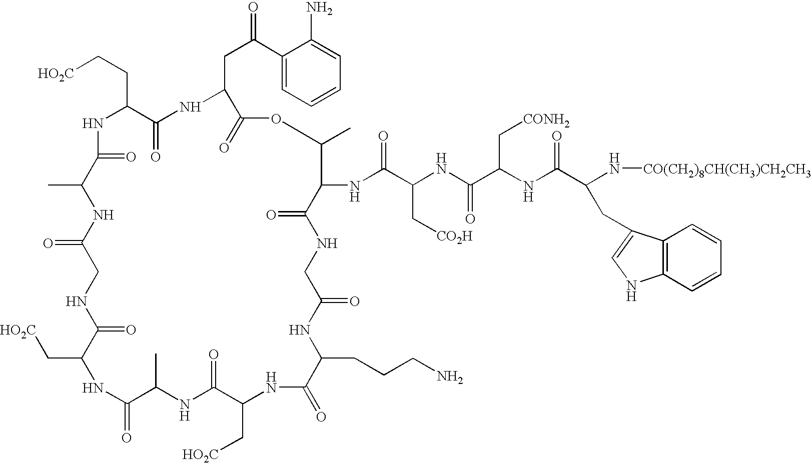 |
| C61 | 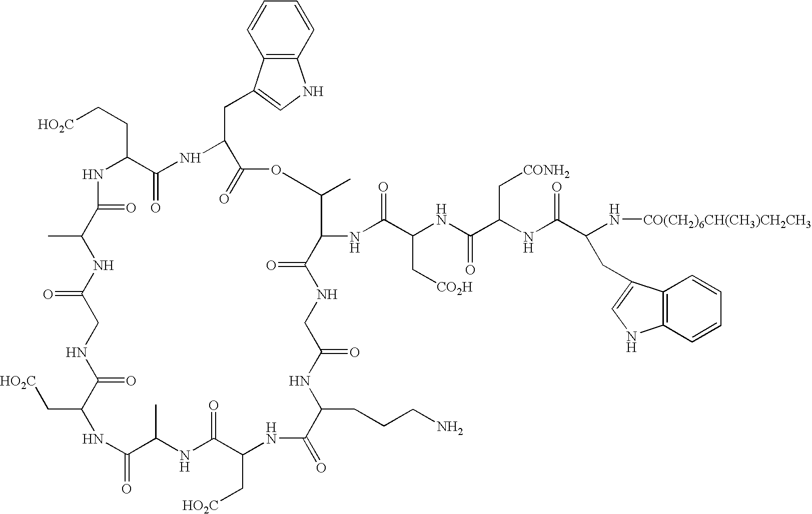 |
| C62 | 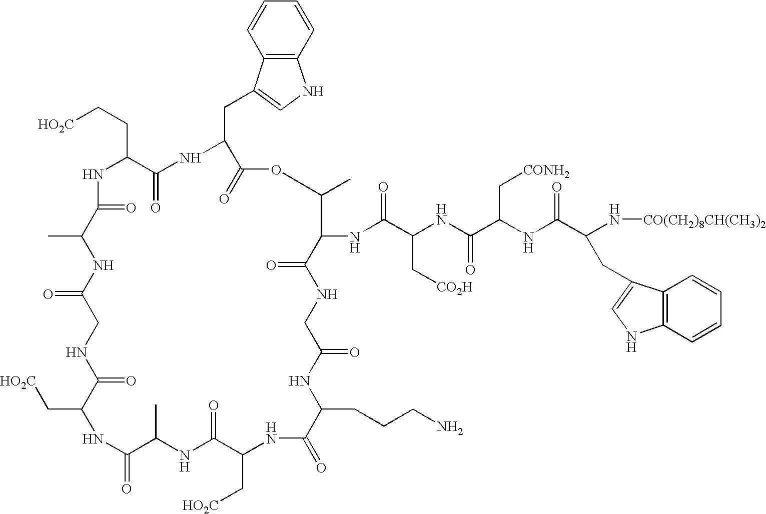 |
| C63 | 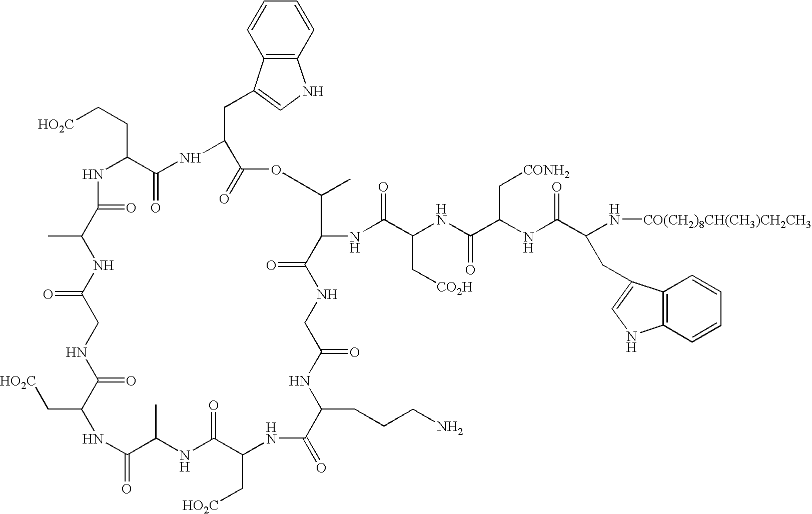 |
| C64 | 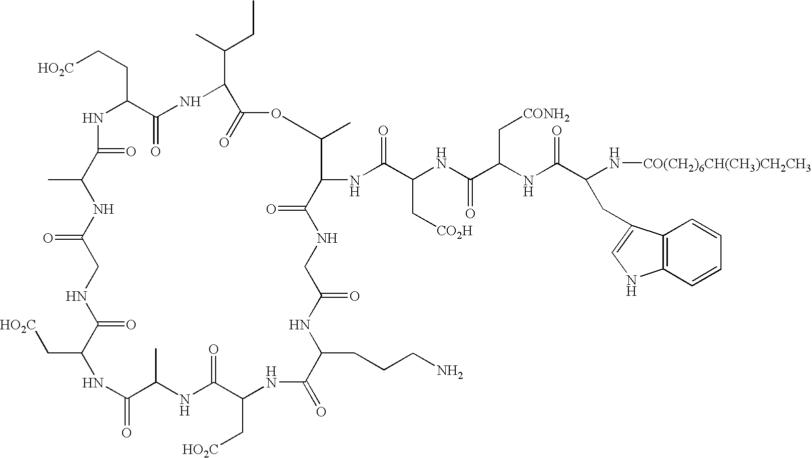 |
| C65 | 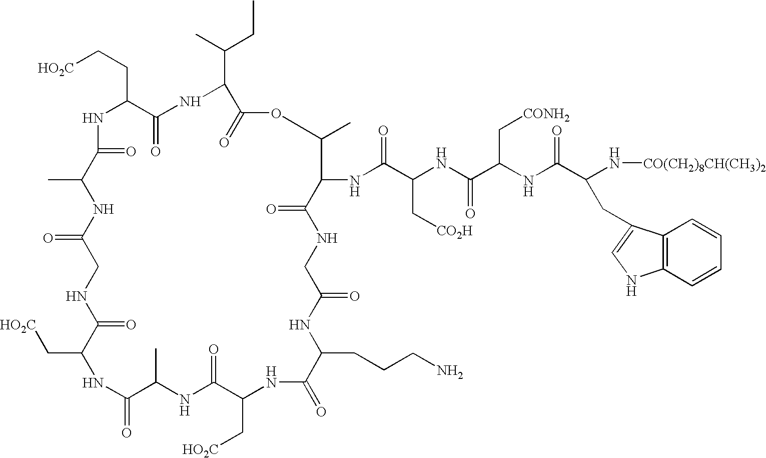 |
| C66 | 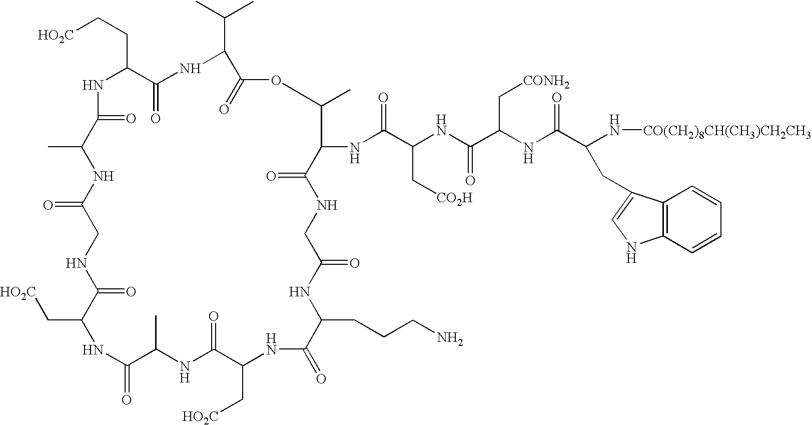 |
| C67 | 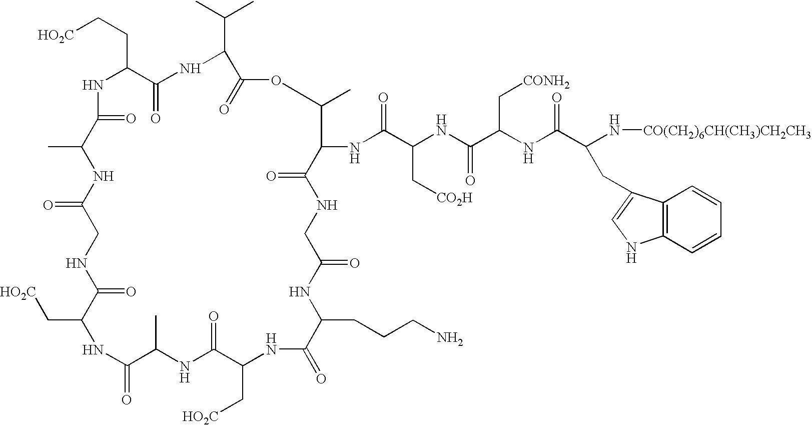 |
| C68 | 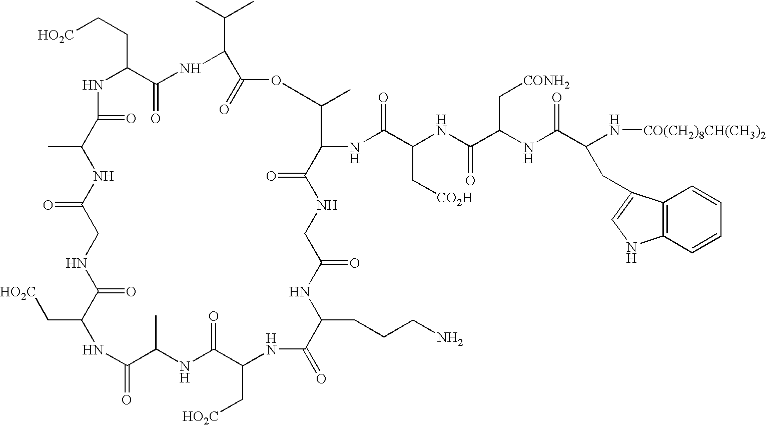 |
| C69 | 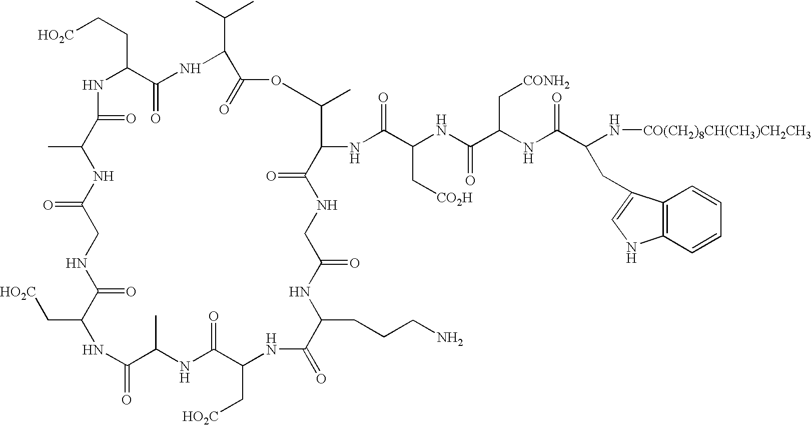 |
| C72 | 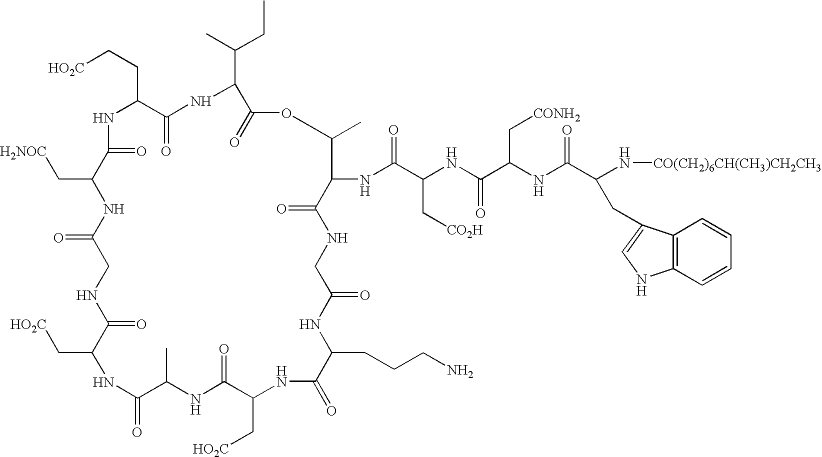 |
| C73 | 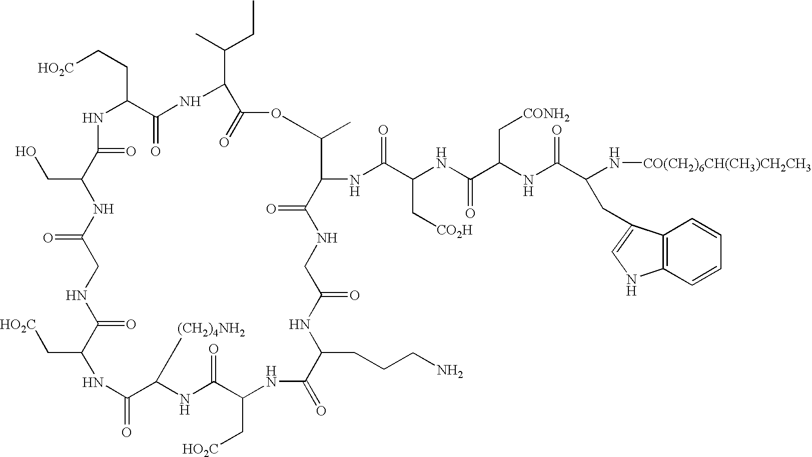 |
| C74 | 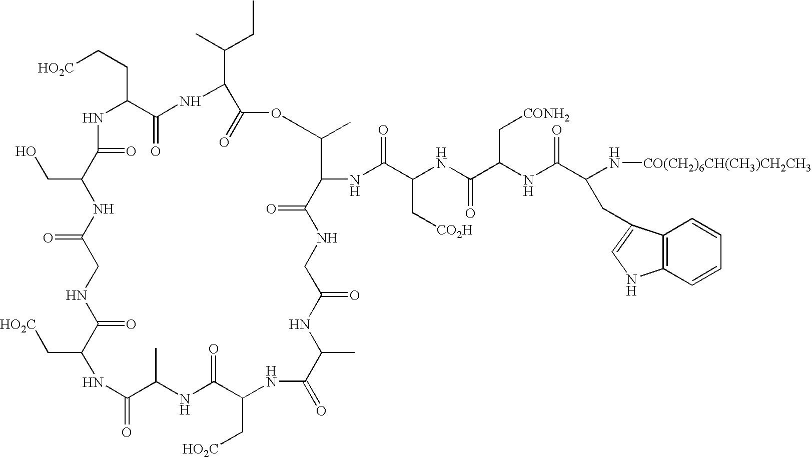 |
| C75 | 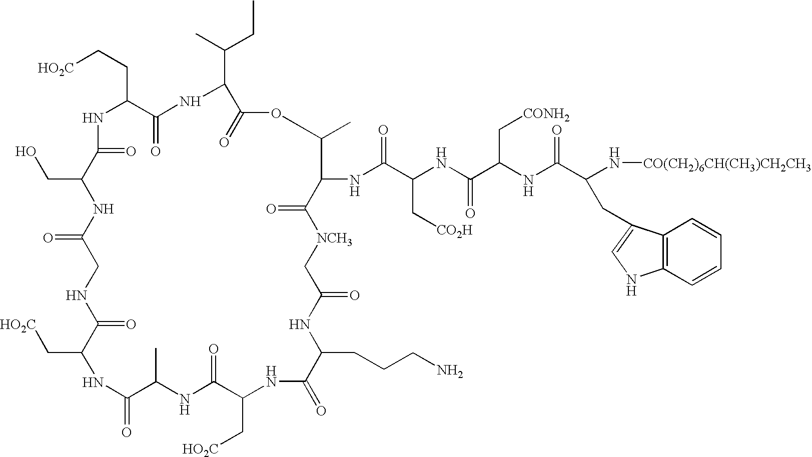 |
| C76 | 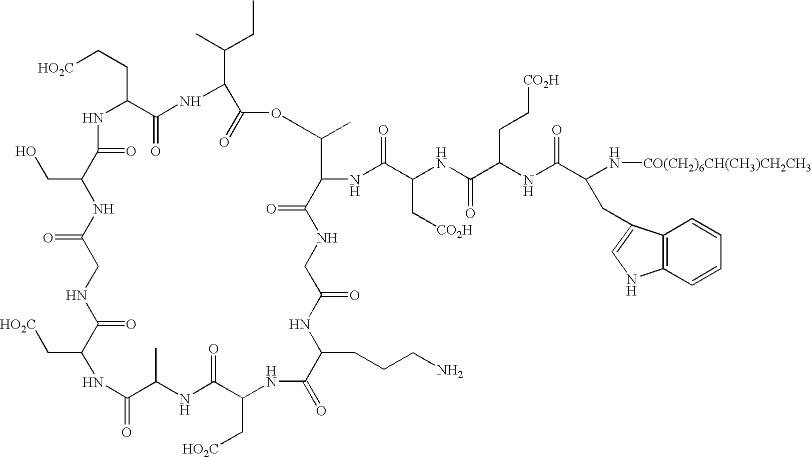 |
| C77 | 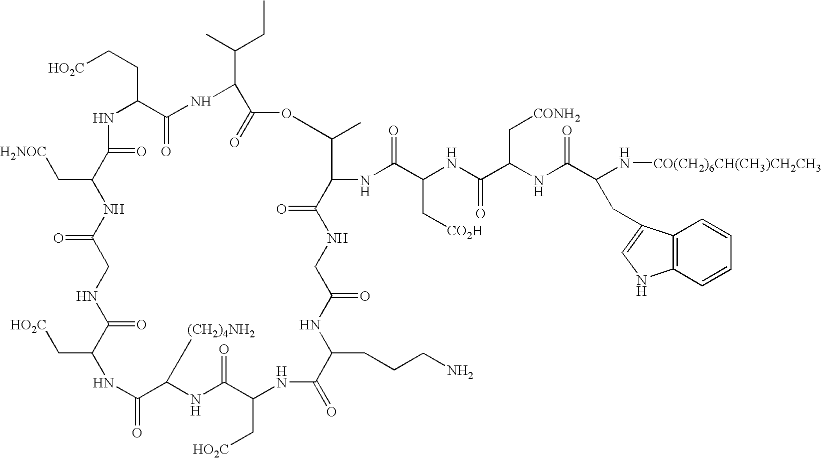 |
| C78 | 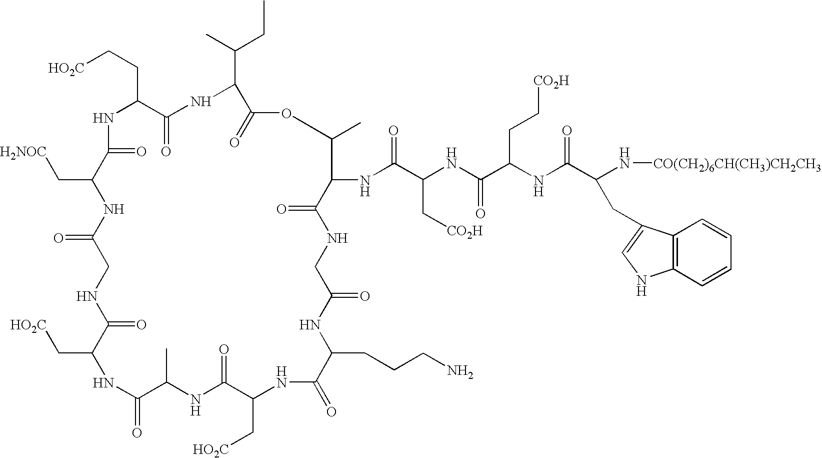 |
| C79 | 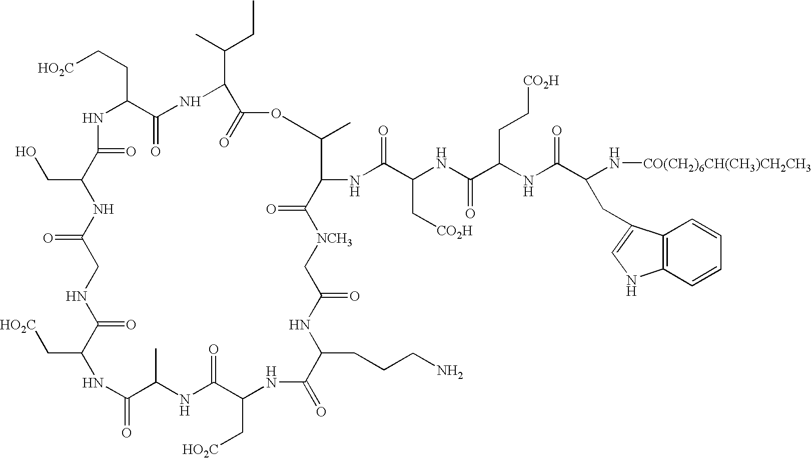 |
| C80 | 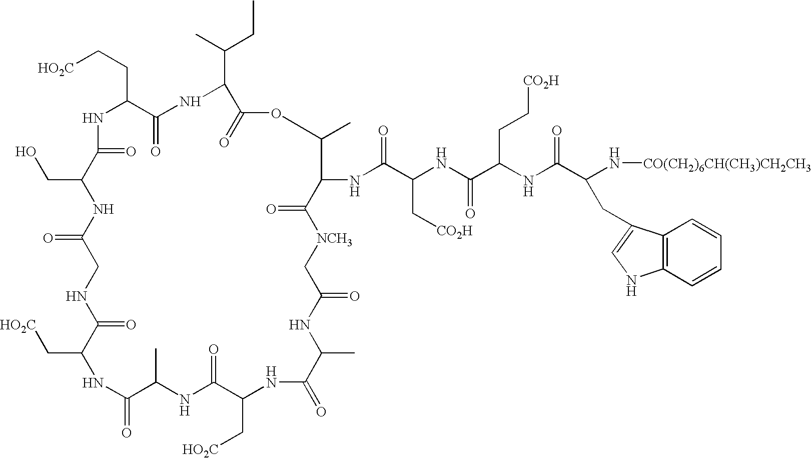 |
| C81 | 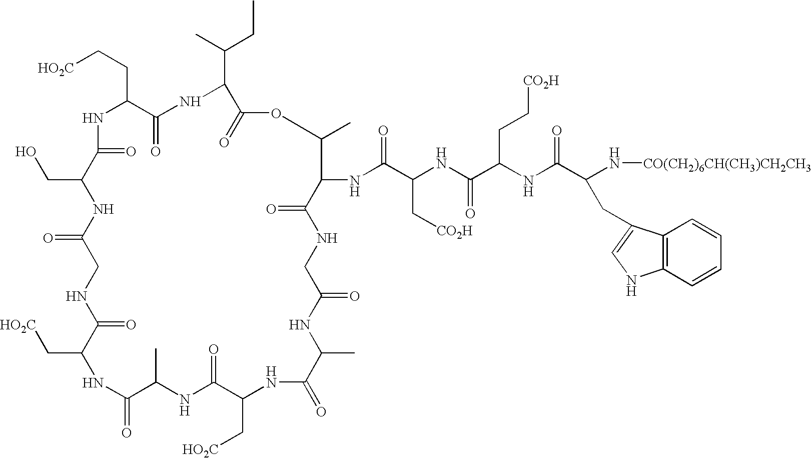 |
| C82 | 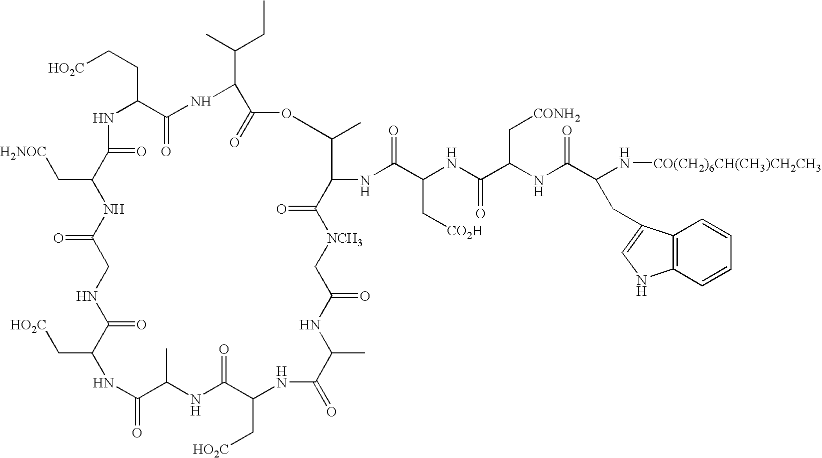 |
| C83 | 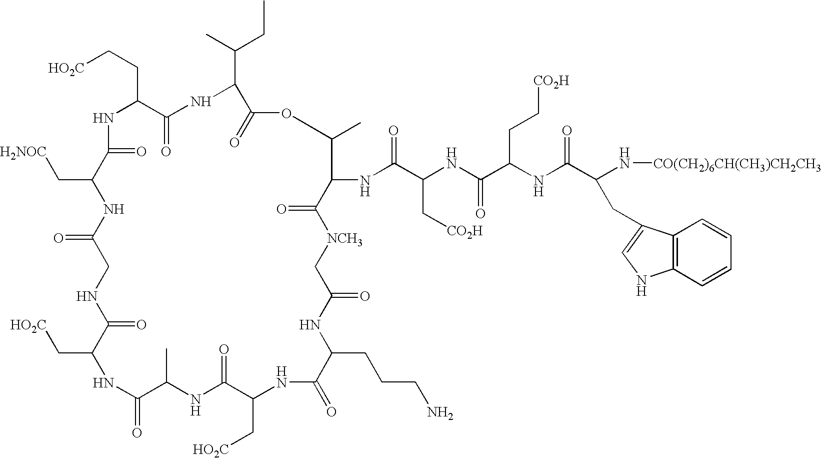 |
| C84 | 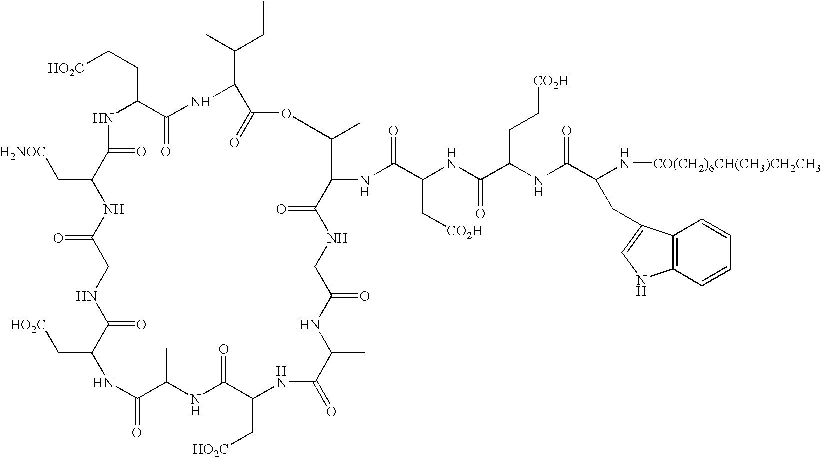 |
| C85 | 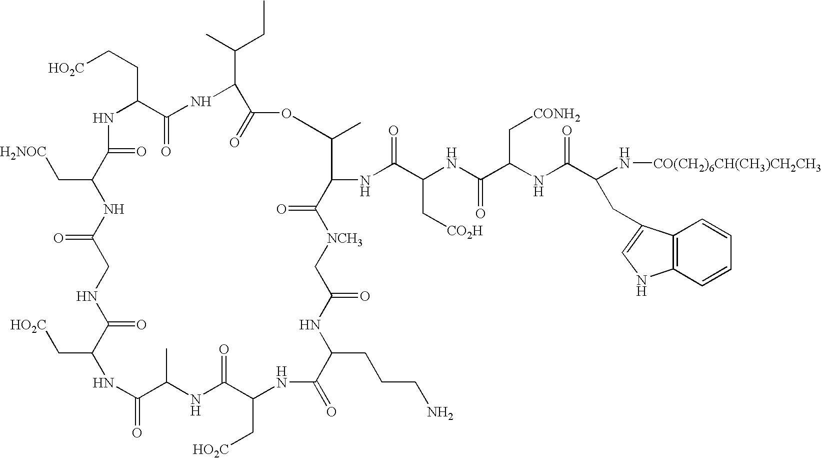 |
| C86 | 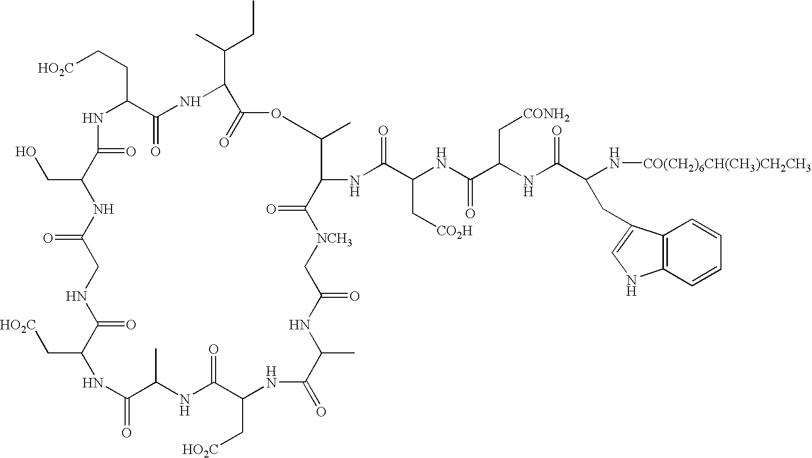 |
| C87 | 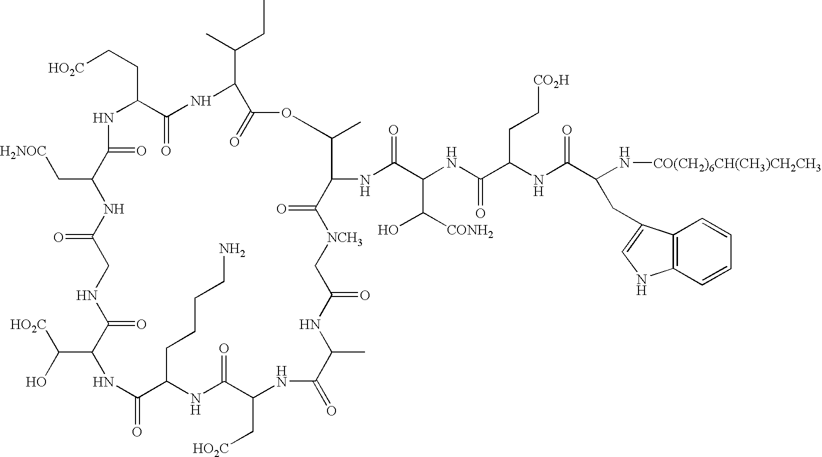 |
| C88 | 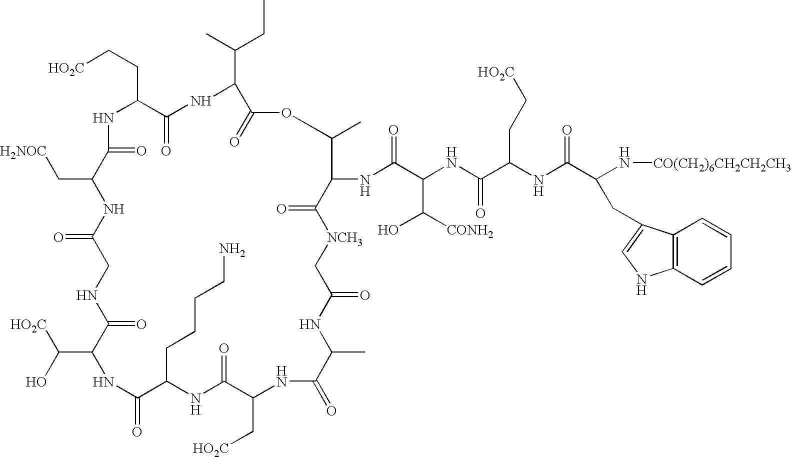 |
| C89 | 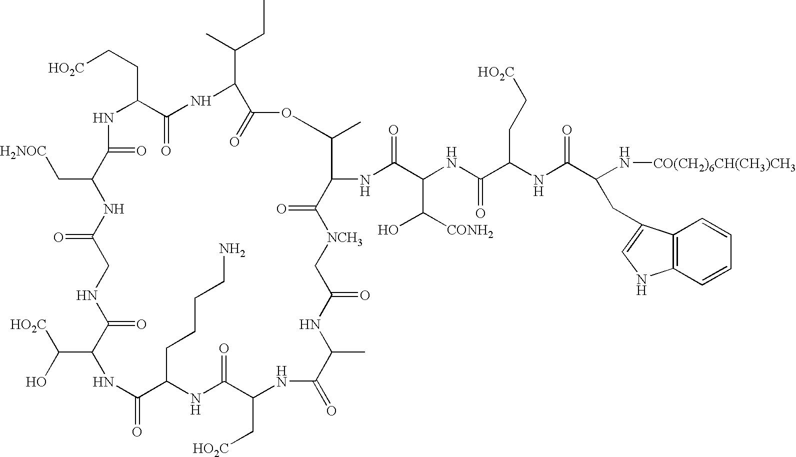 |
| C90 | 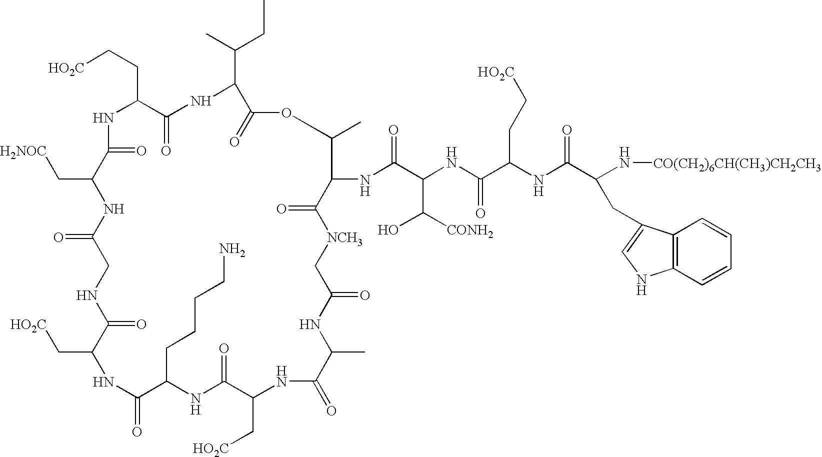 |
| C91 | 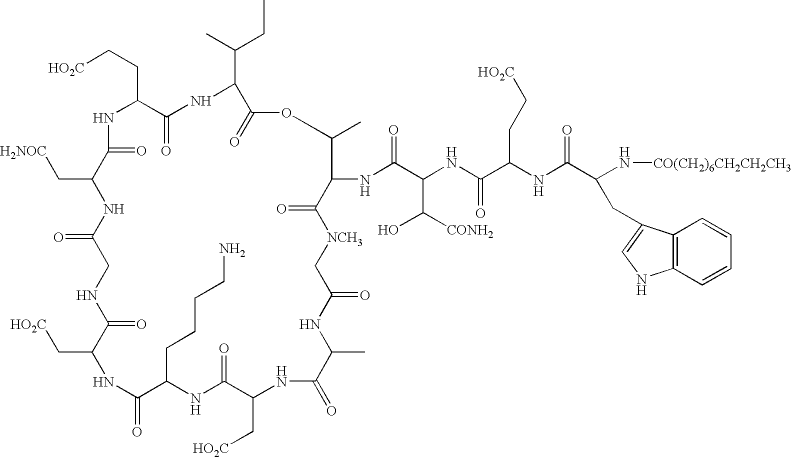 |
| C92 | 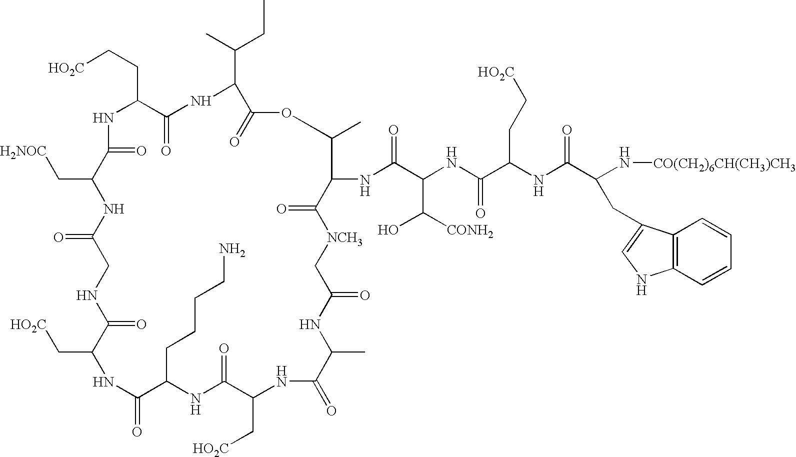 |
| C93 | 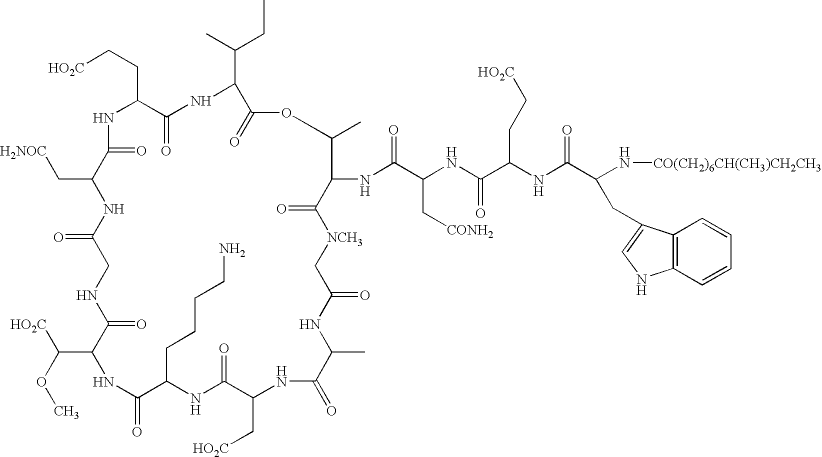 |
| C94 | 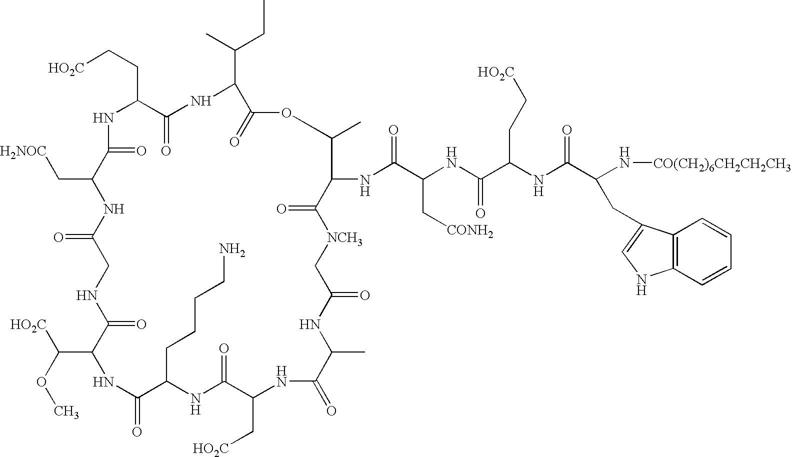 |
| C95 | 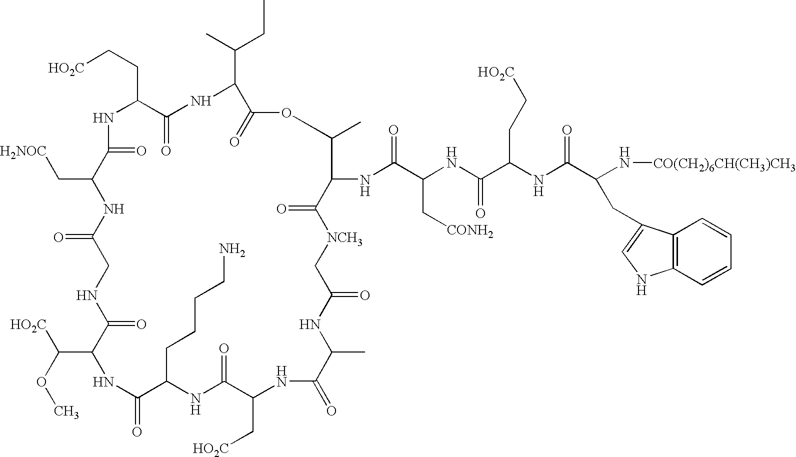 |
| C96 | 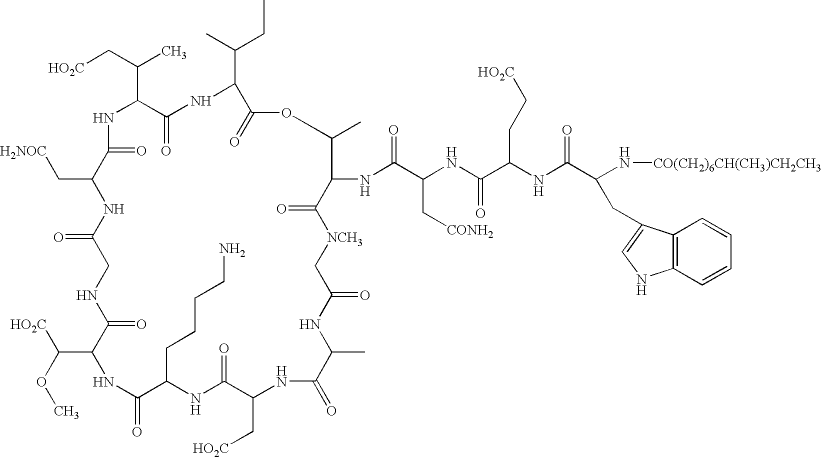 |
| C97 | 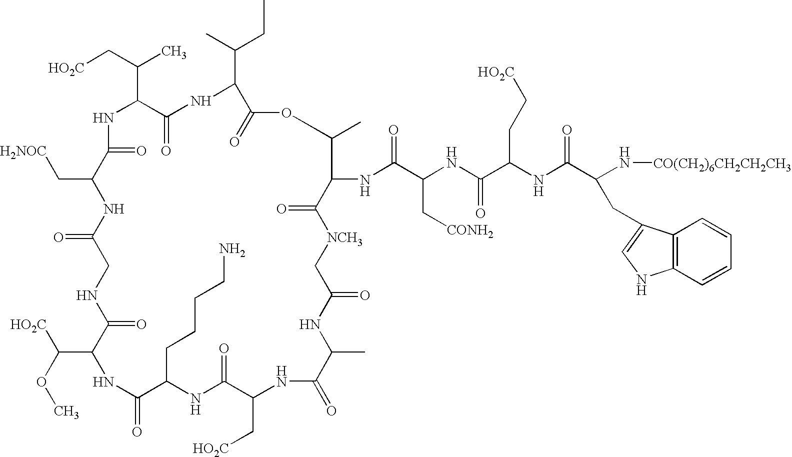 |
| C98 | 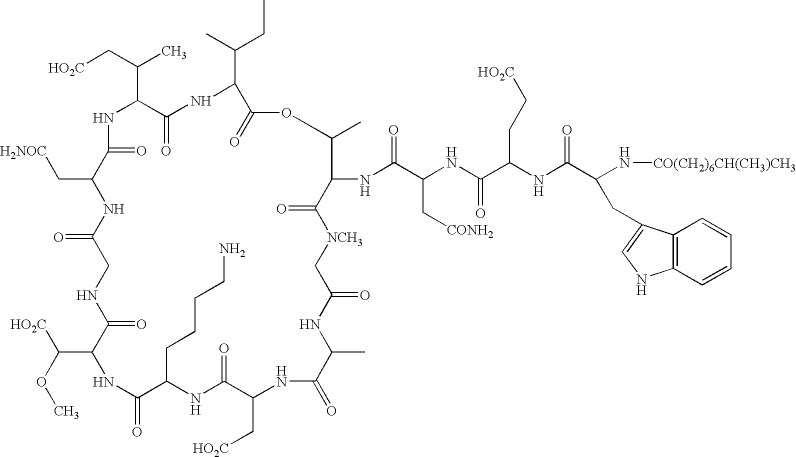 |
| C99 | 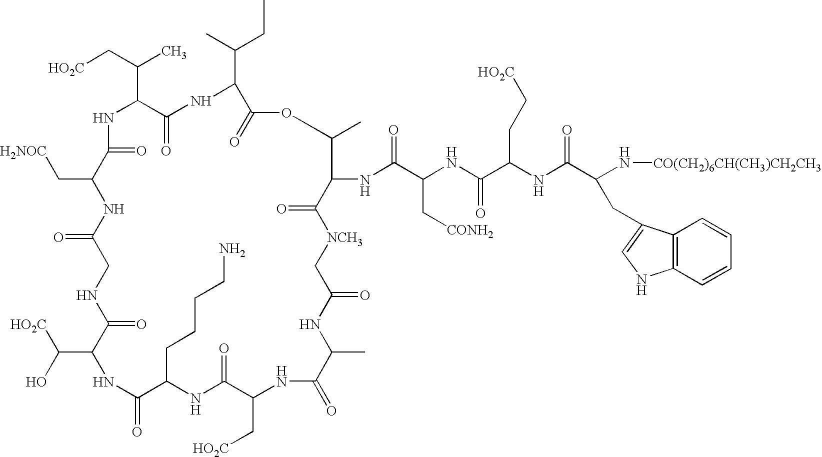 |
| C100 | 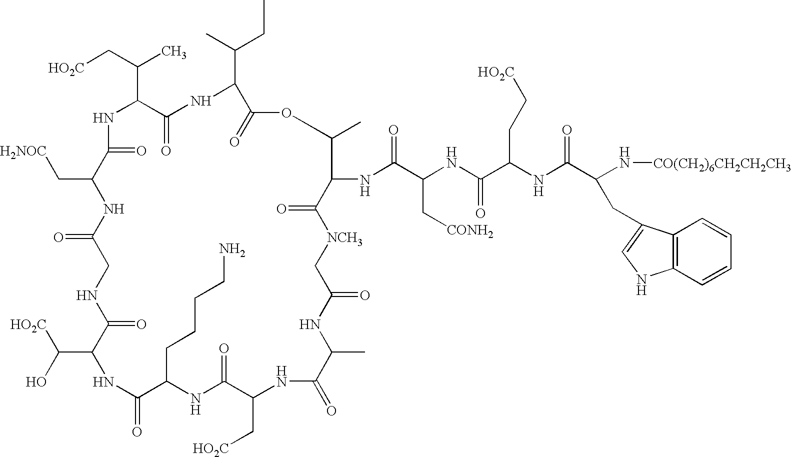 |
| C101 | 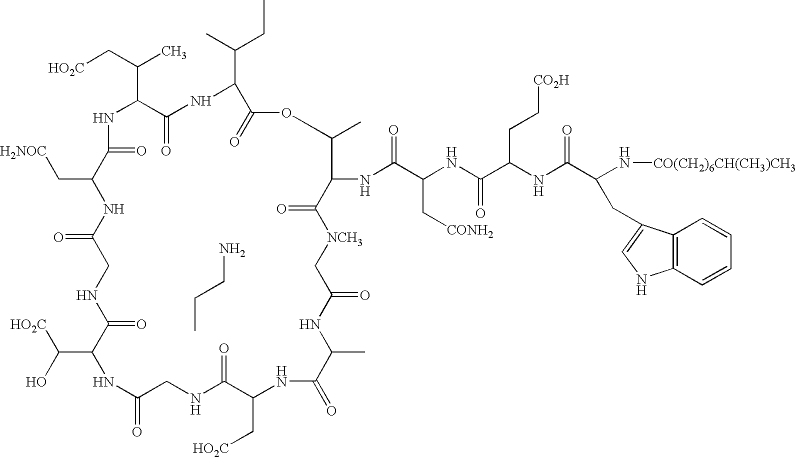 |
| C102 | 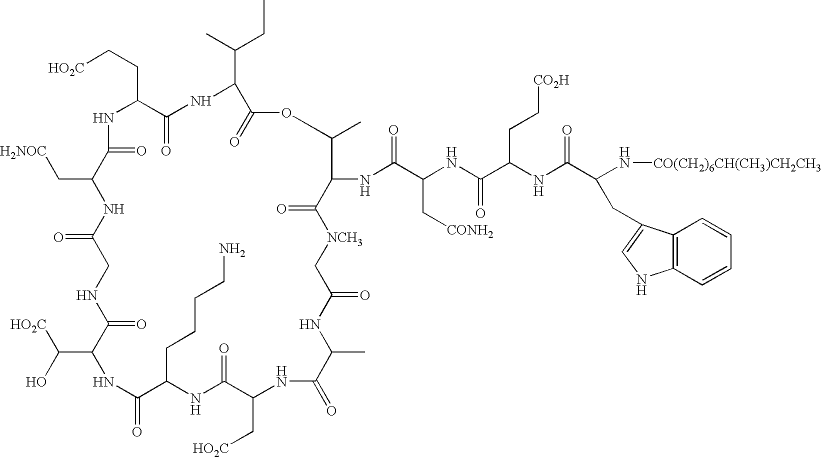 |
| C103 | 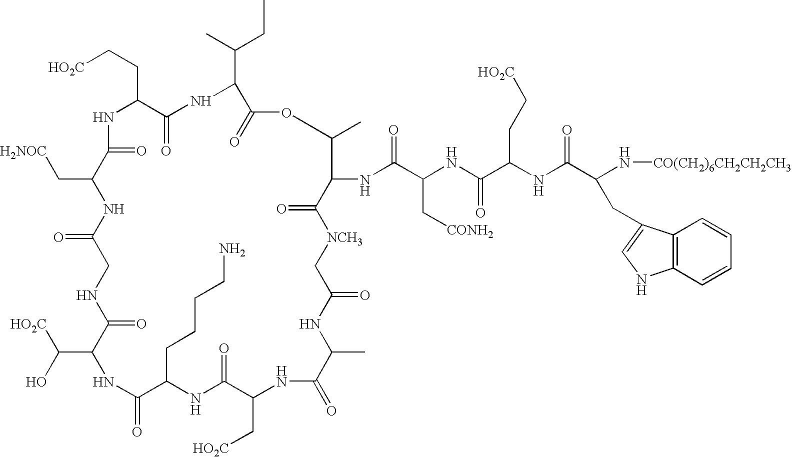 |
| C104 | 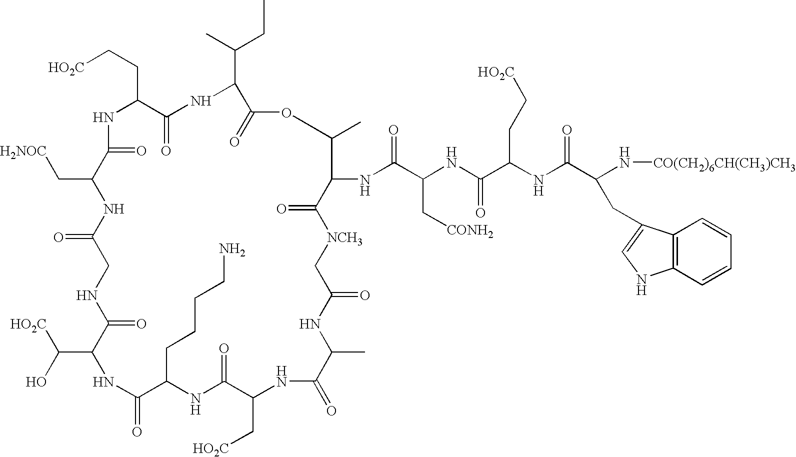 |
| C105 | 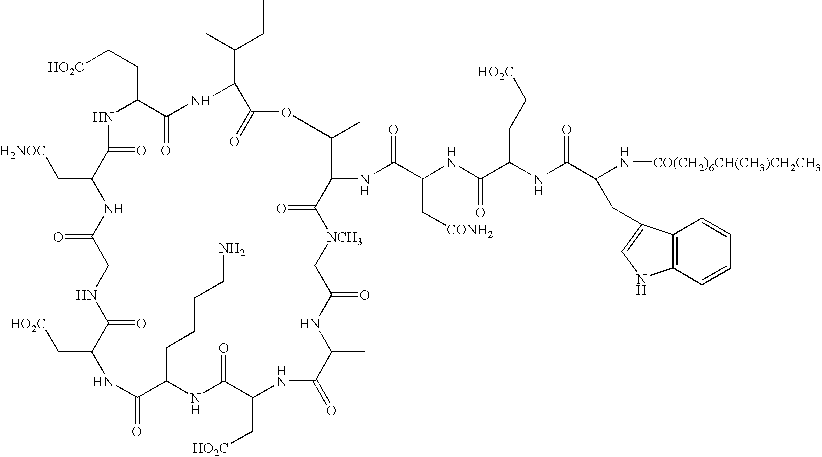 |
| C106 | 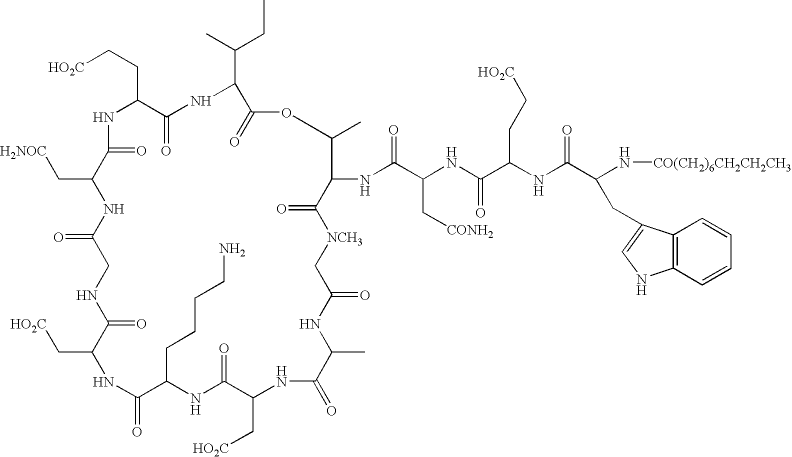 |
| C107 | 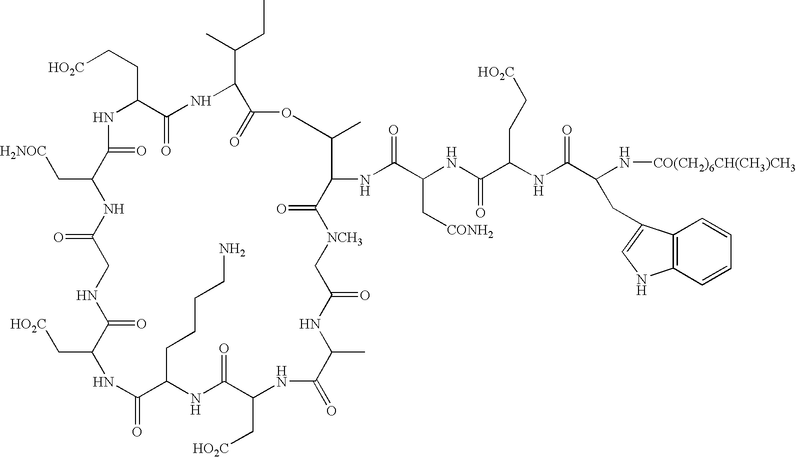 |
| C108 | 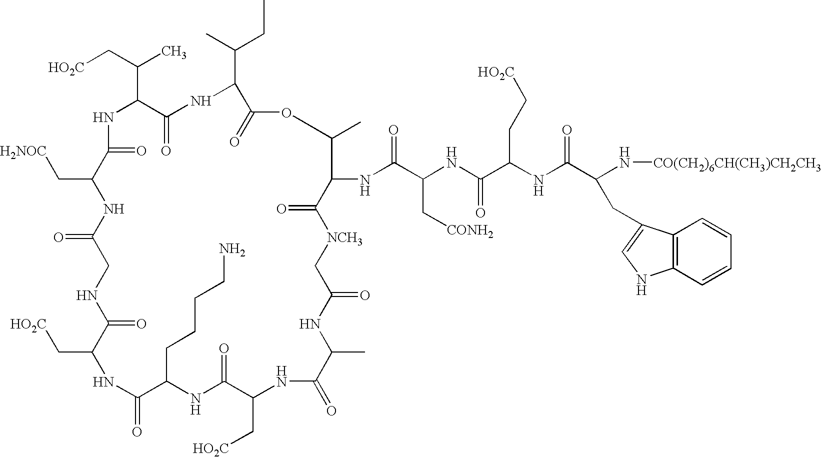 |
| C109 | 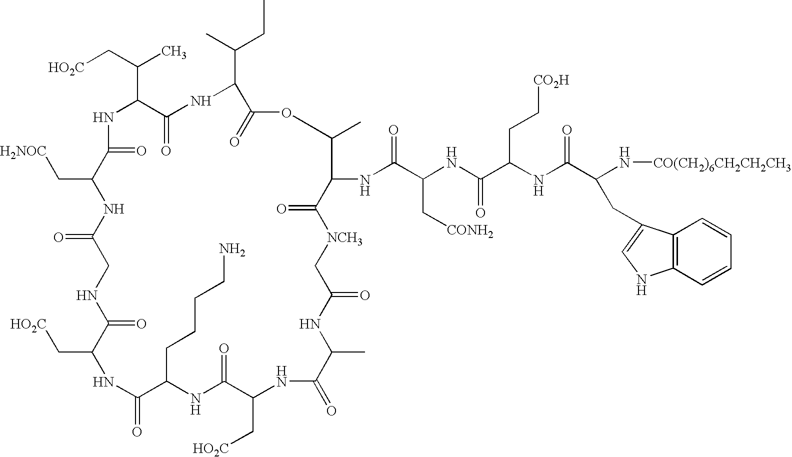 |
| C110 | 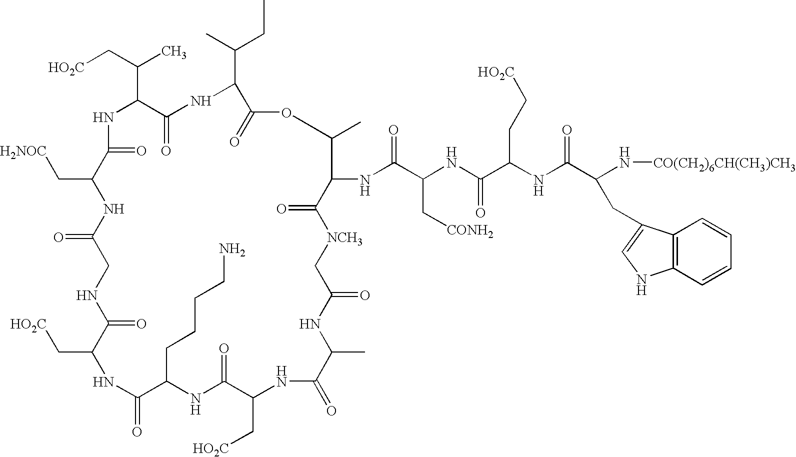 |
| C111 | 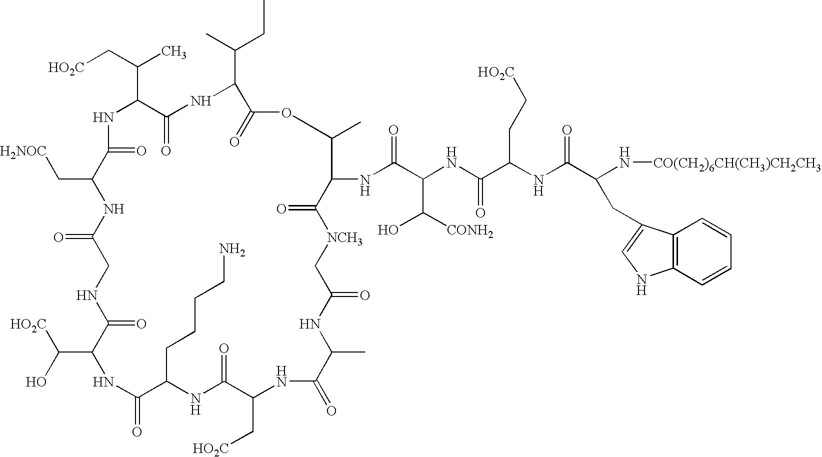 |
| C112 | 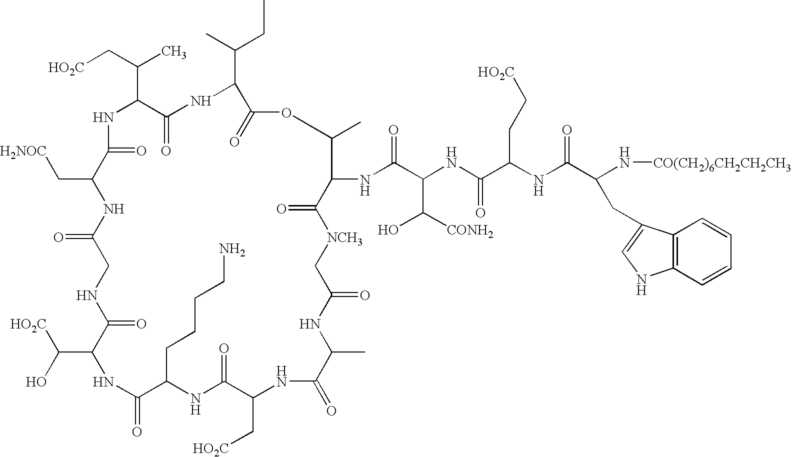 |
| C113 | 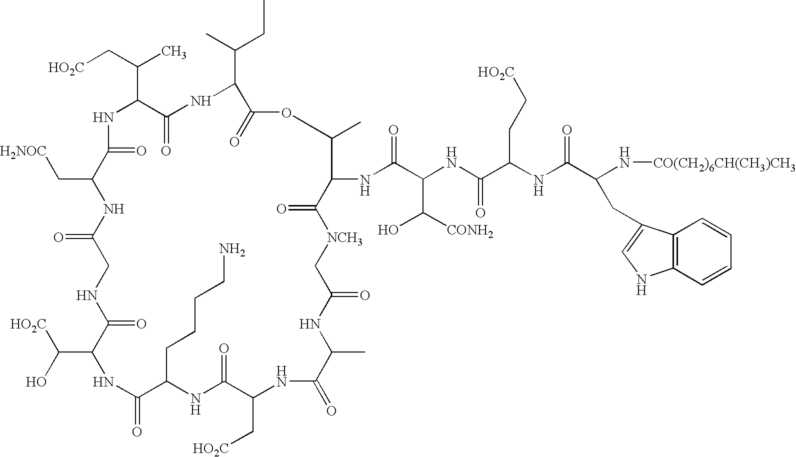 |
| C114 | 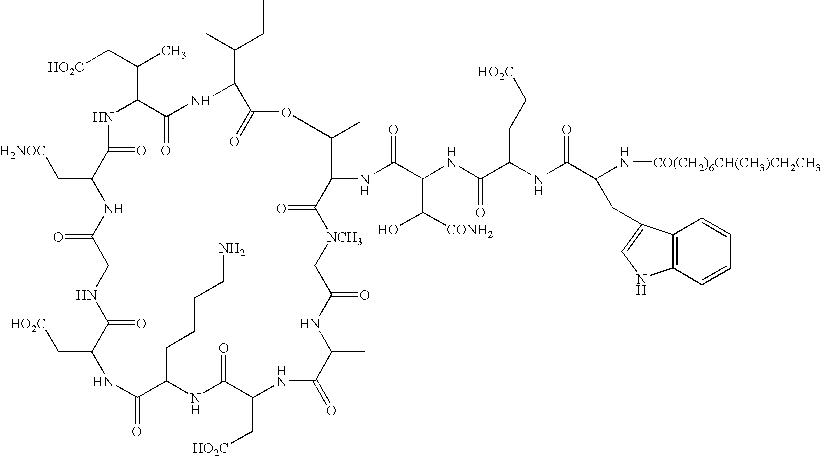 |
| C115 | 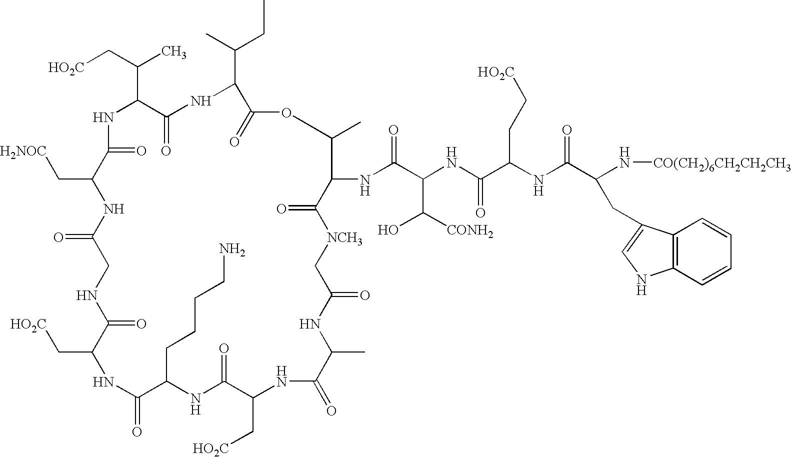 |
| C116 | 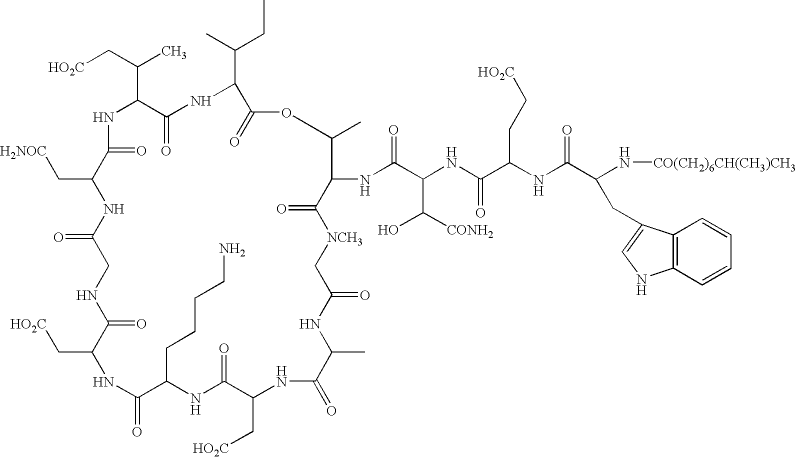 |
| C117 | 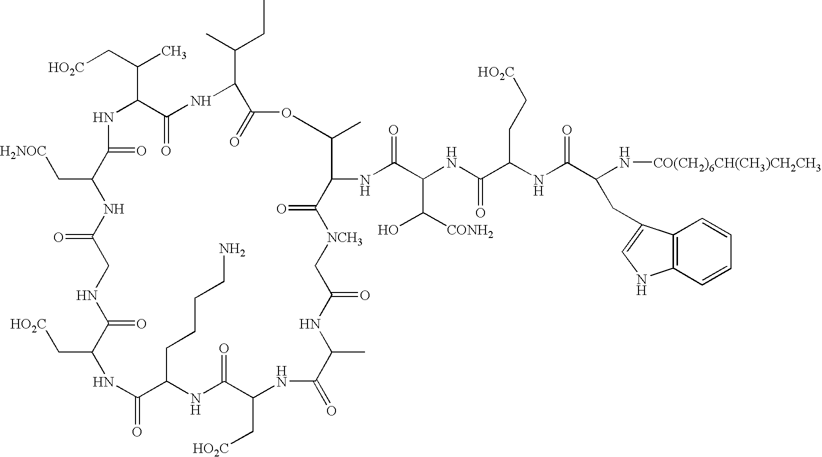 |
| C118 | 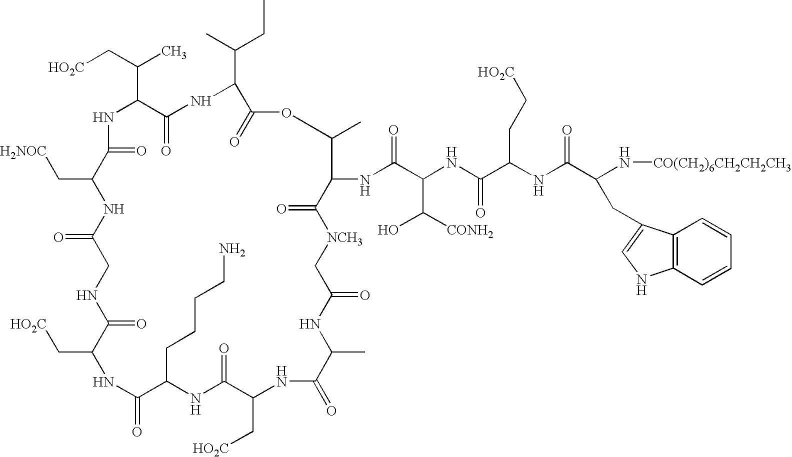 |
| C119 | 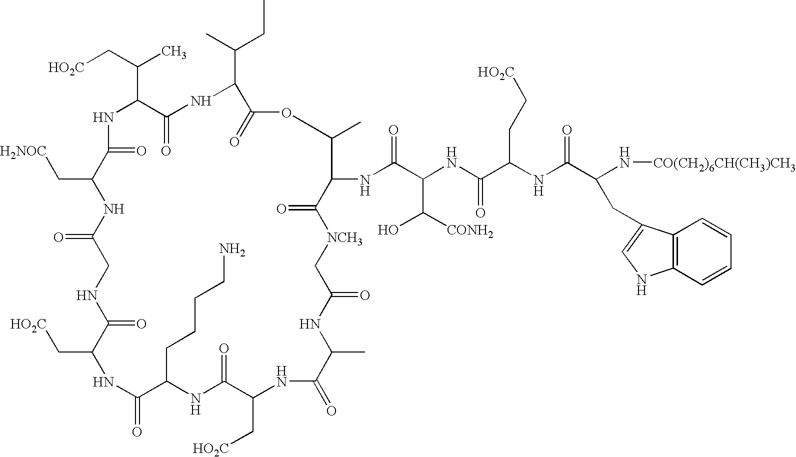 |
| C120 | 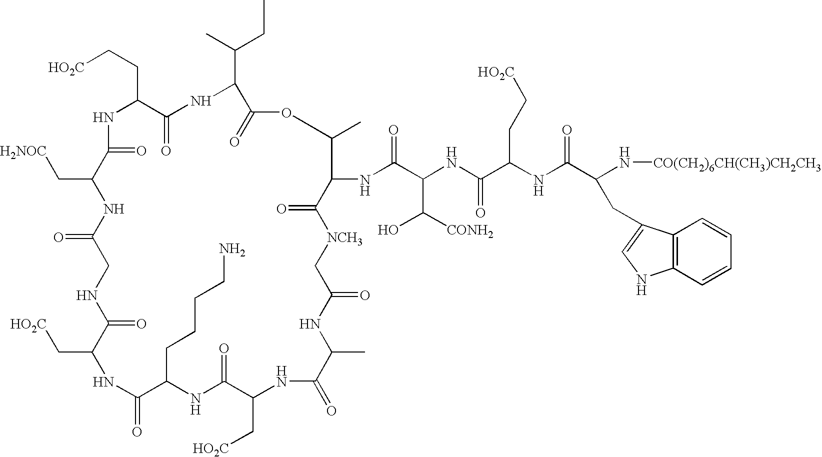 |
| C121 | 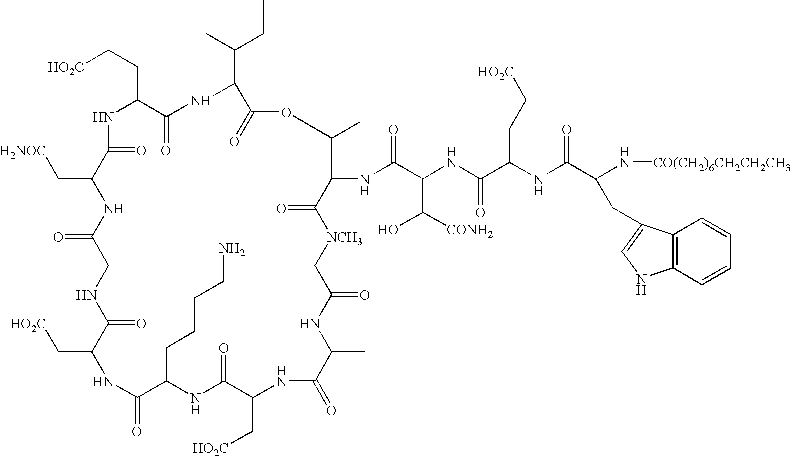 |
| C122 | 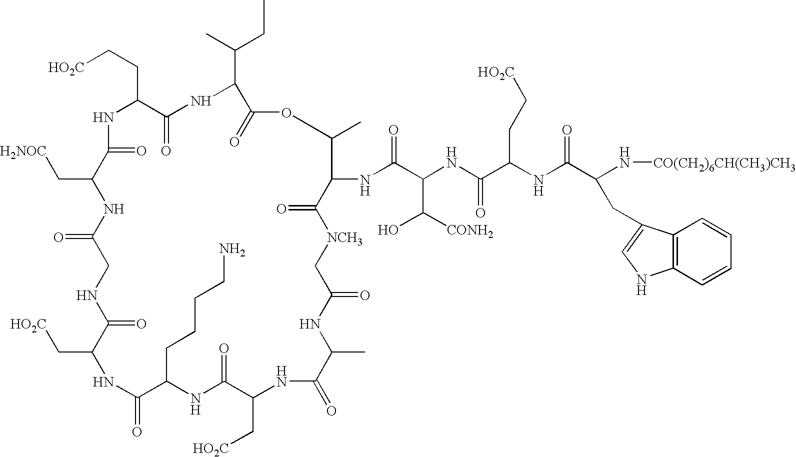 |
| C123 | 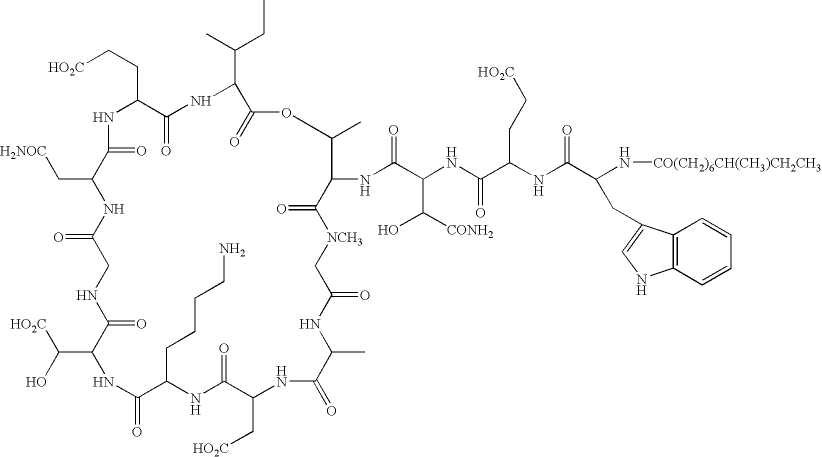 |
| C124 | 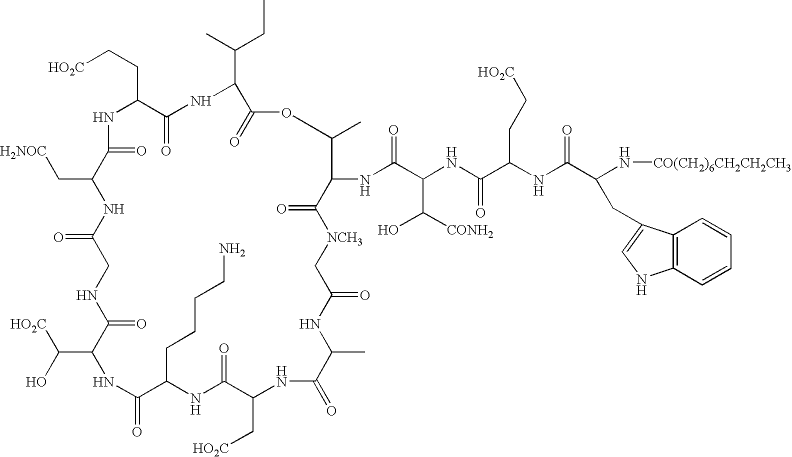 |
| C125 | 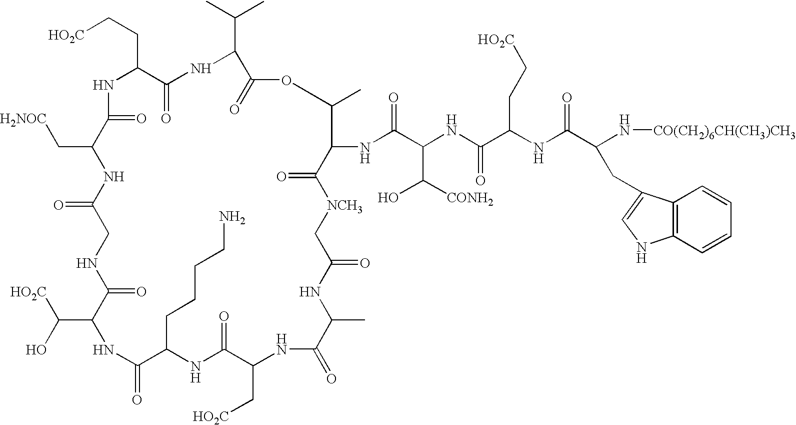 |
| C126 | 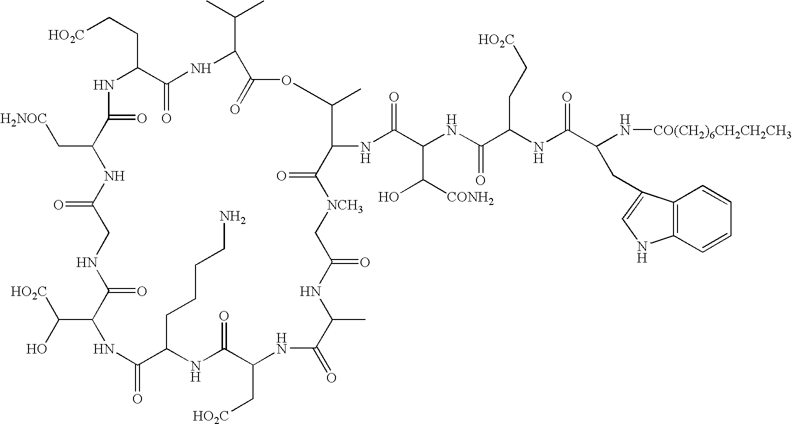 |
| C127 | 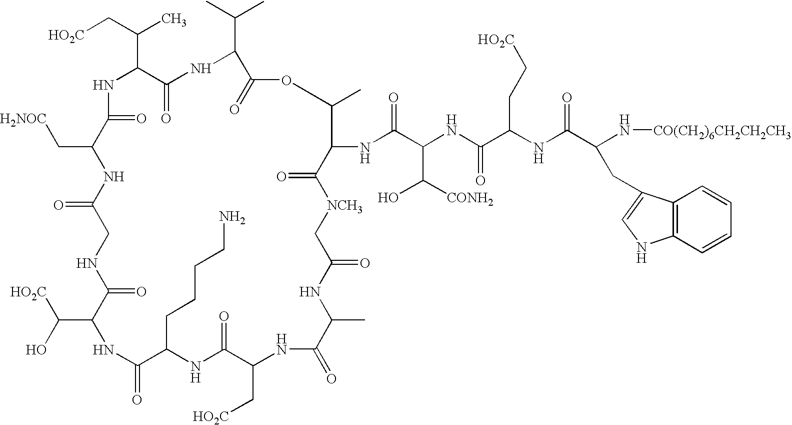 |
| C128 | 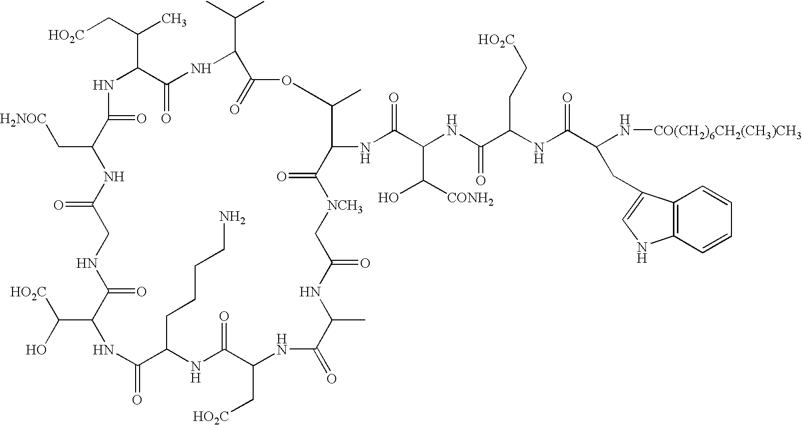 |
| C129 | 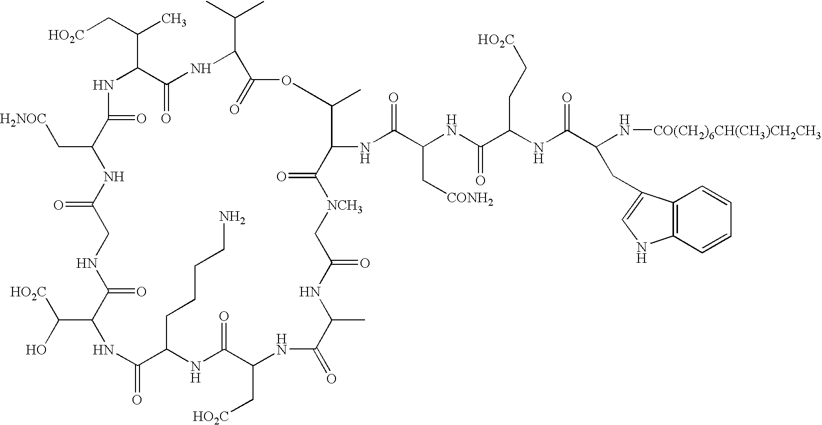 |
| C130 | 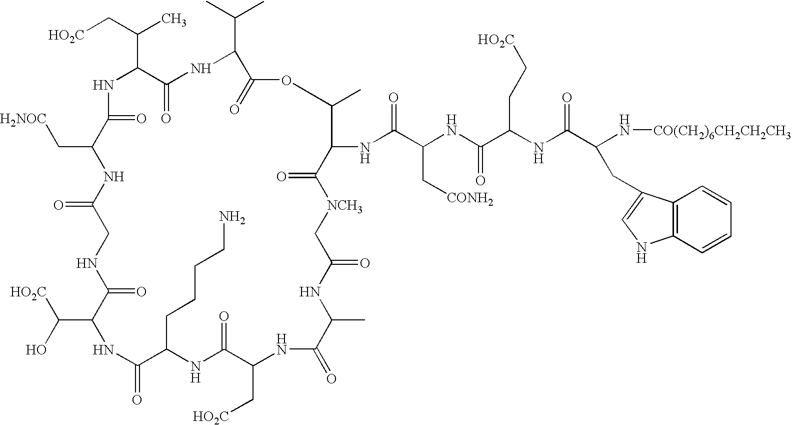 |
| C131 | 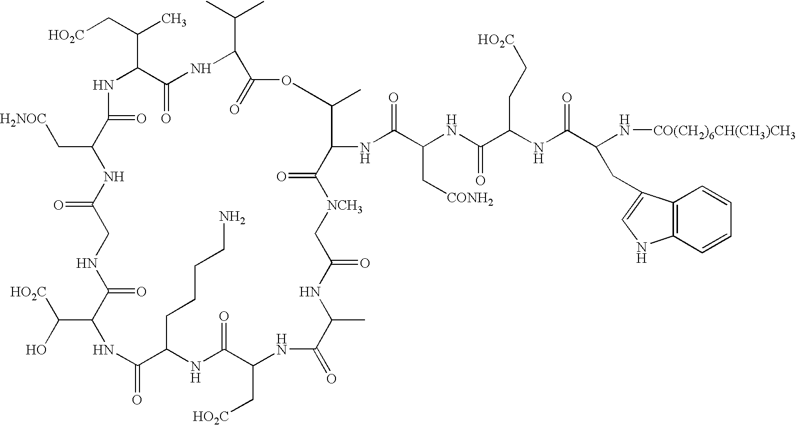 |
| C132 | 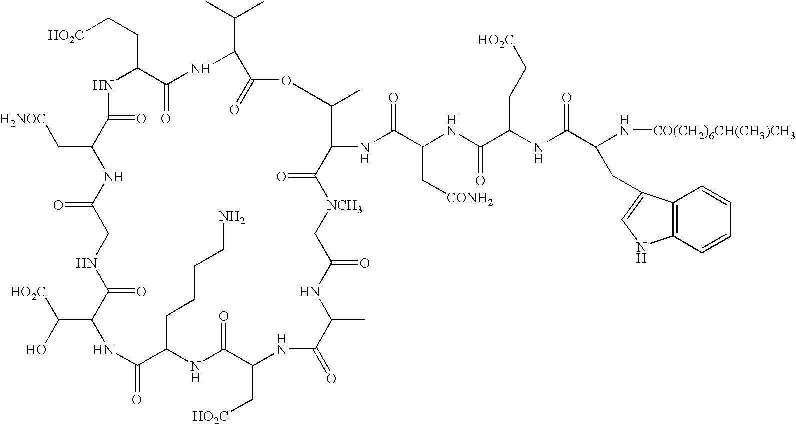 |
| C133 | 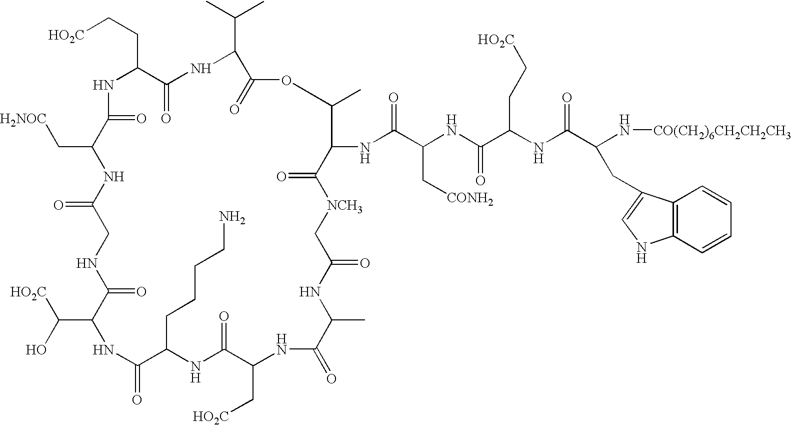 |
| C134 | 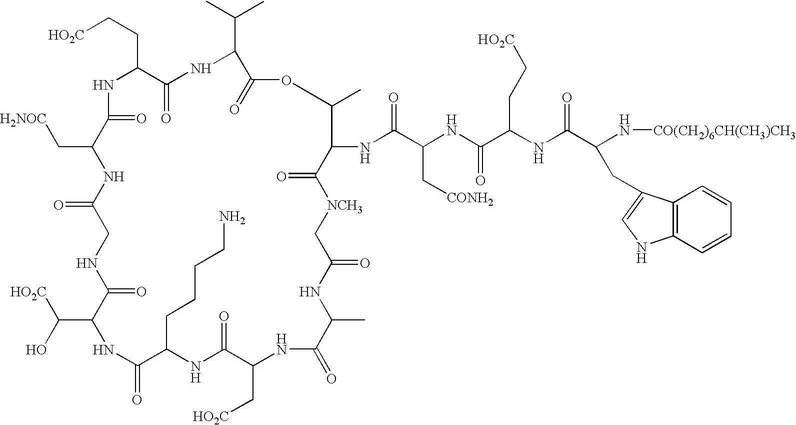 |
| C135 | 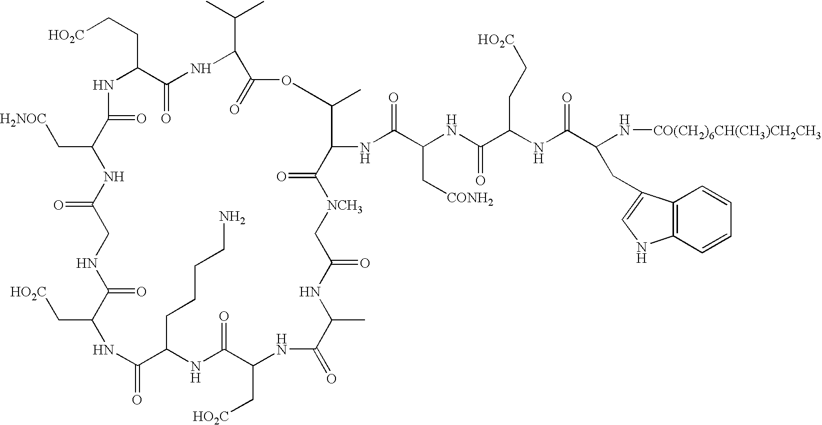 |
| C136 | 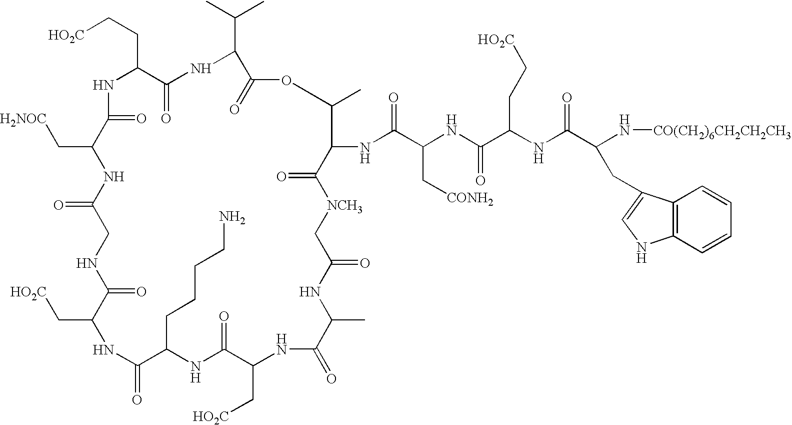 |
| C137 | 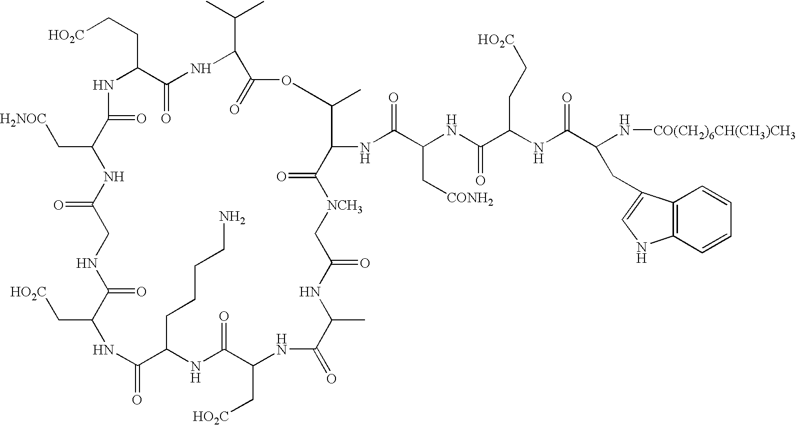 |
| C138 | 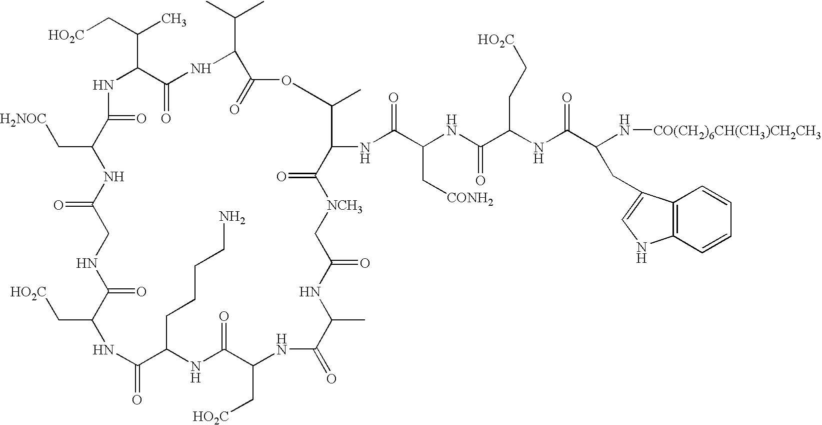 |
| C139 | 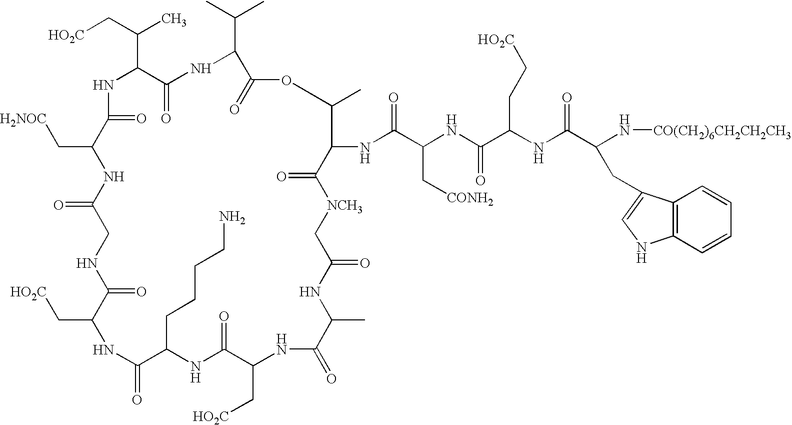 |
| C140 | 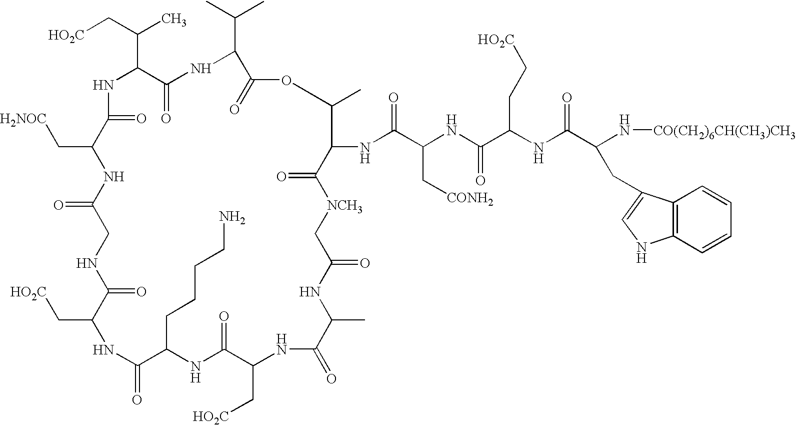 |
| C141 | 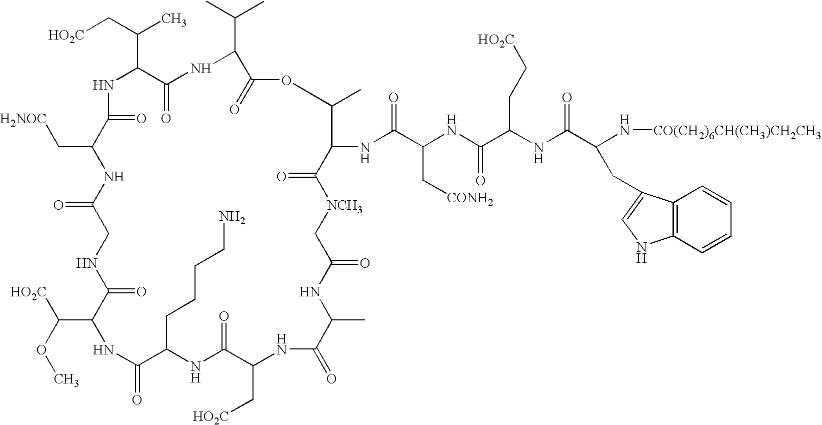 |
| C142 | 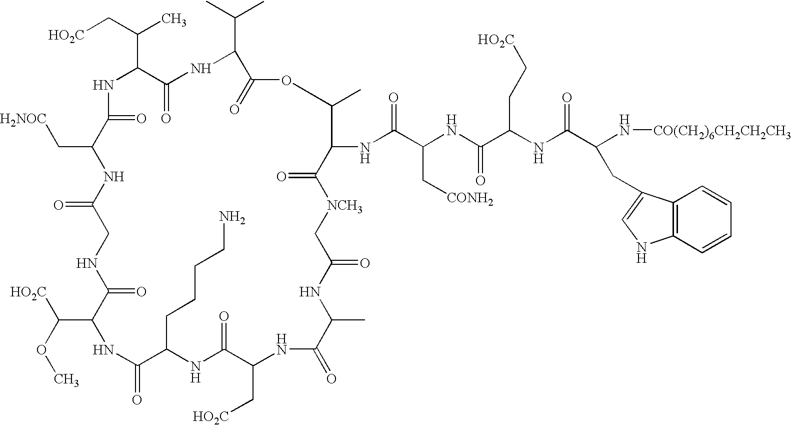 |
| C143 | 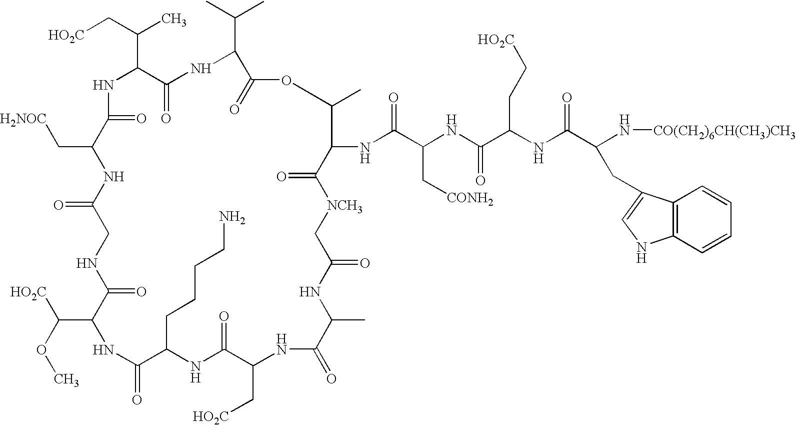 |
| C144 | 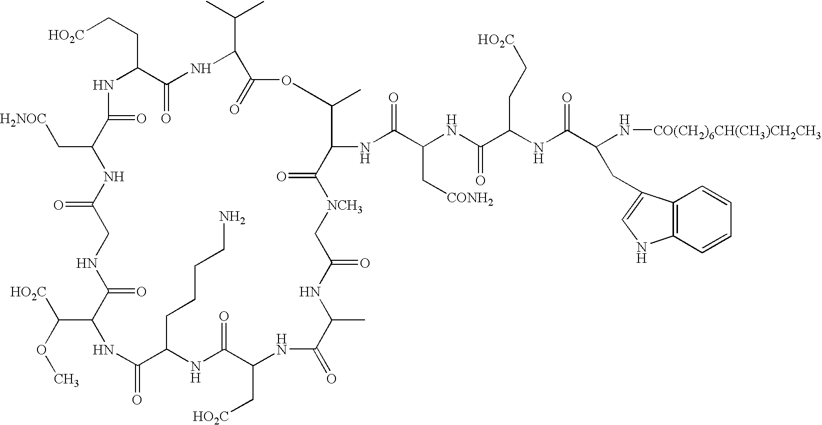 |
| C145 | 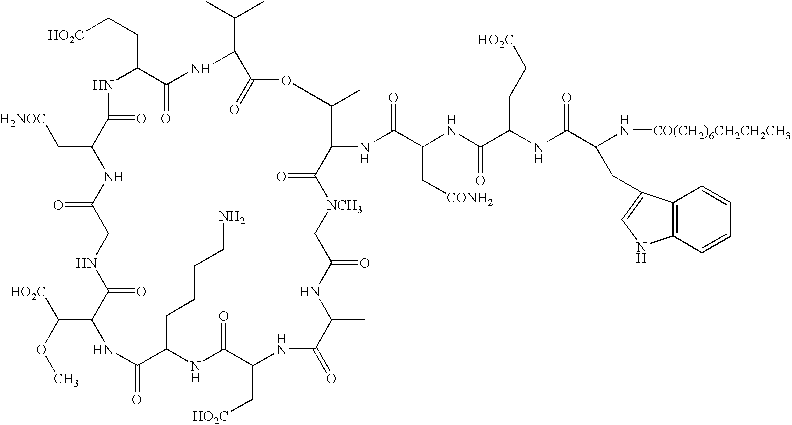 |
| C146 | 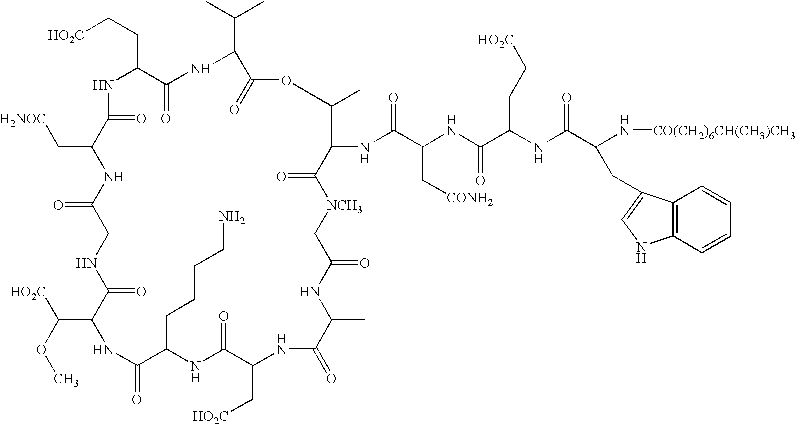 |
| C147 | 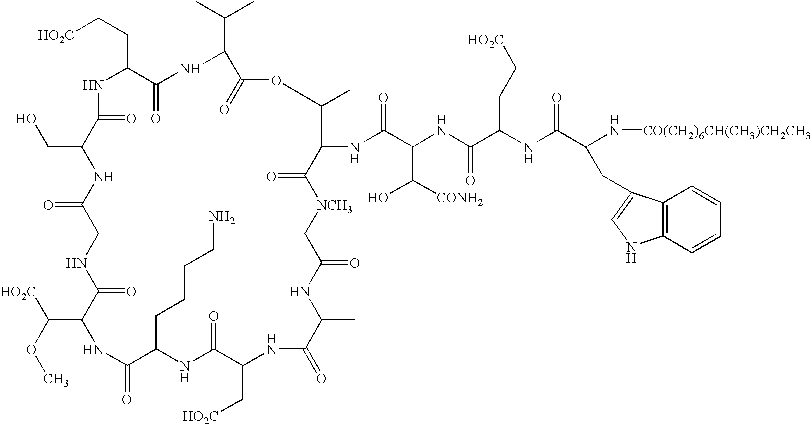 |
| C148 | 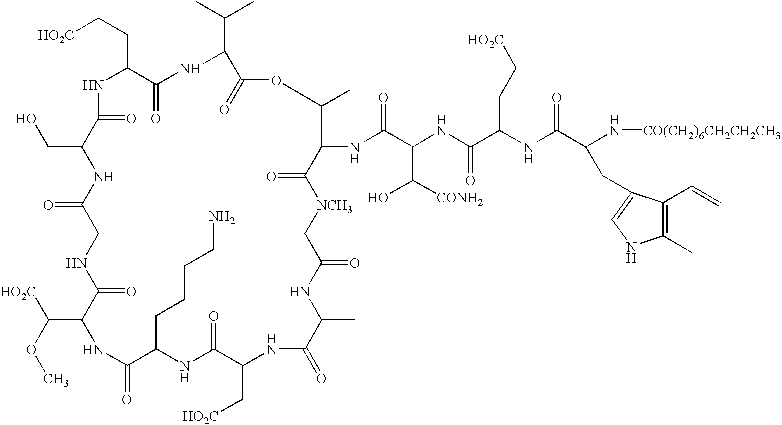 |
| C149 | 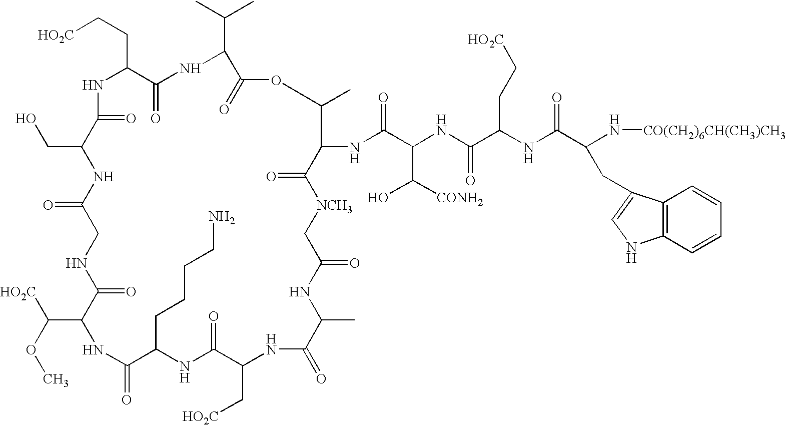 |
| C150 | 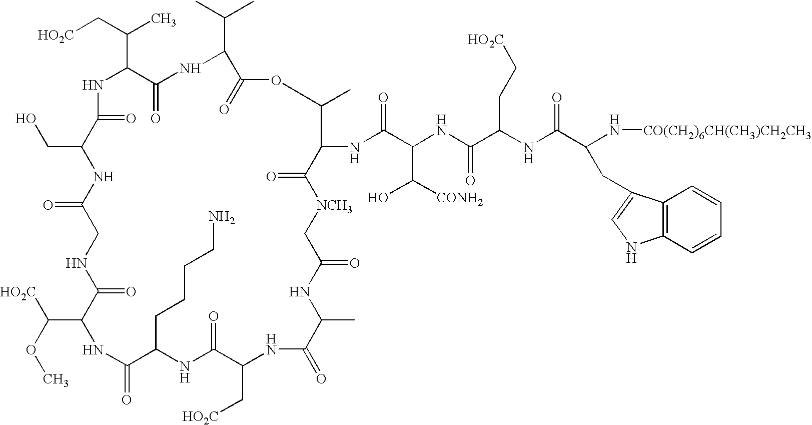 |
| C151 | 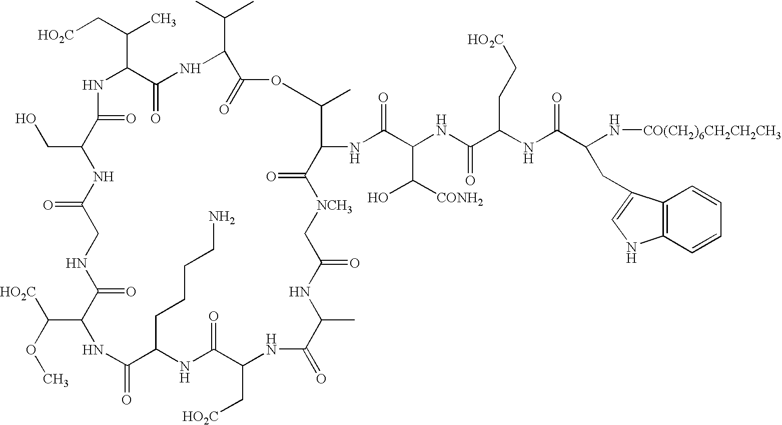 |
| C152 | 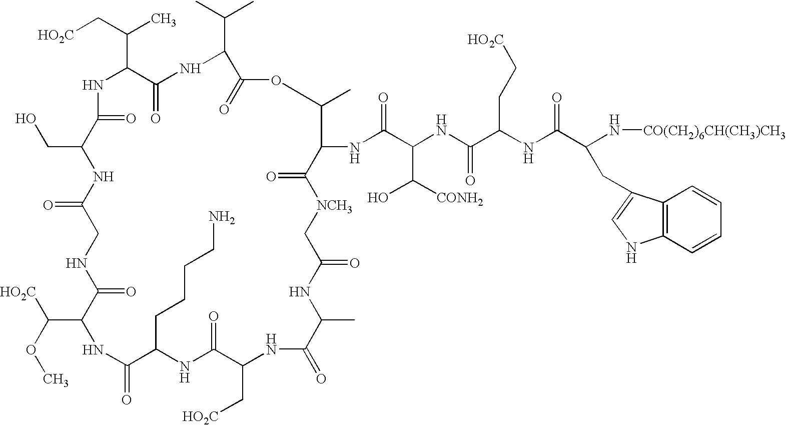 |
| C153 | 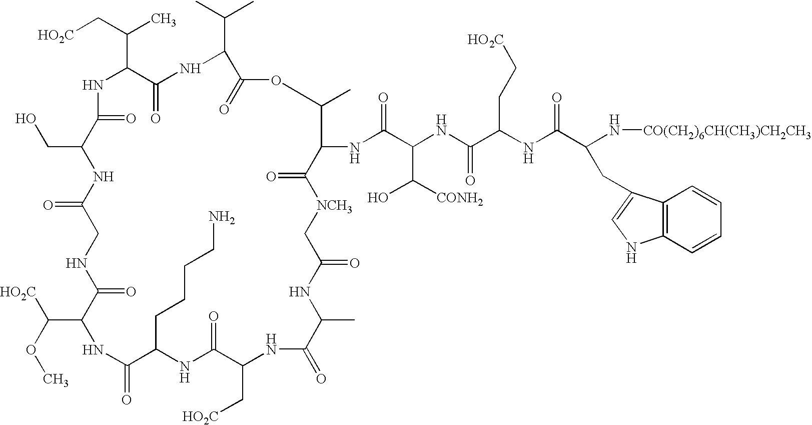 |
| C154 | 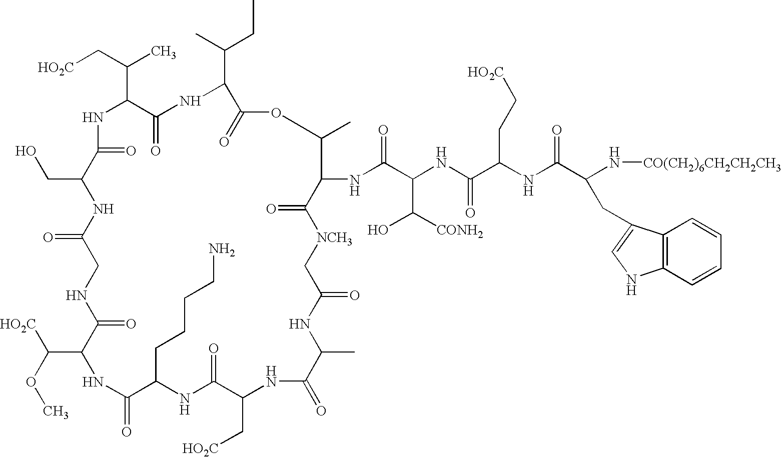 |
| C155 | 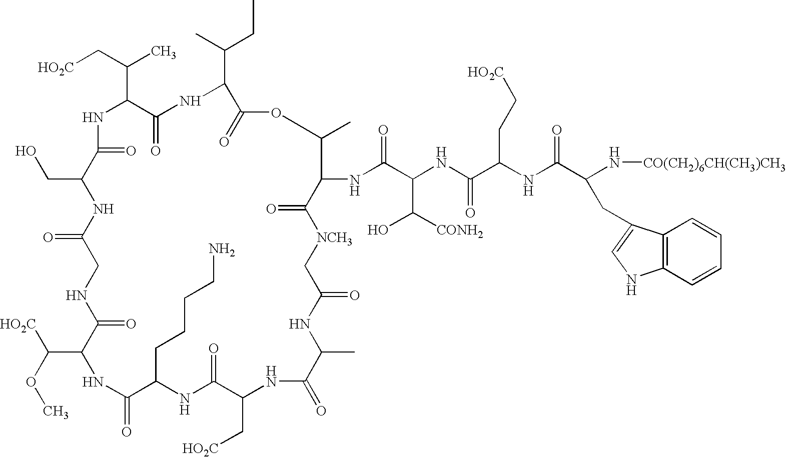 |
| C180 | 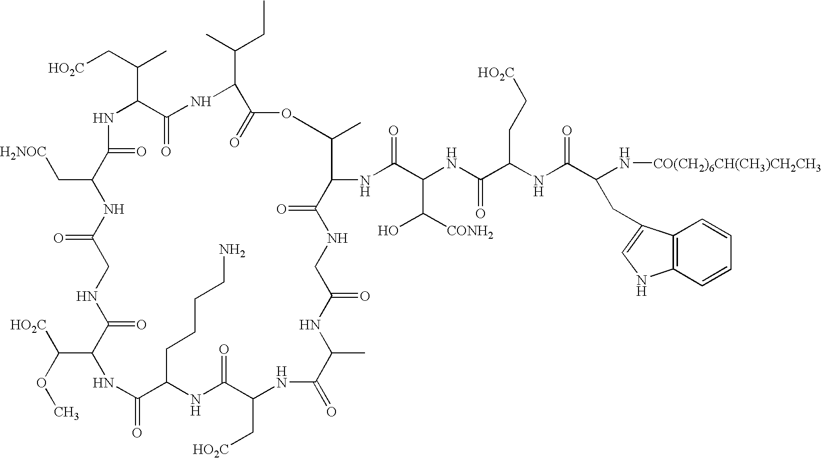 |
| C181 | 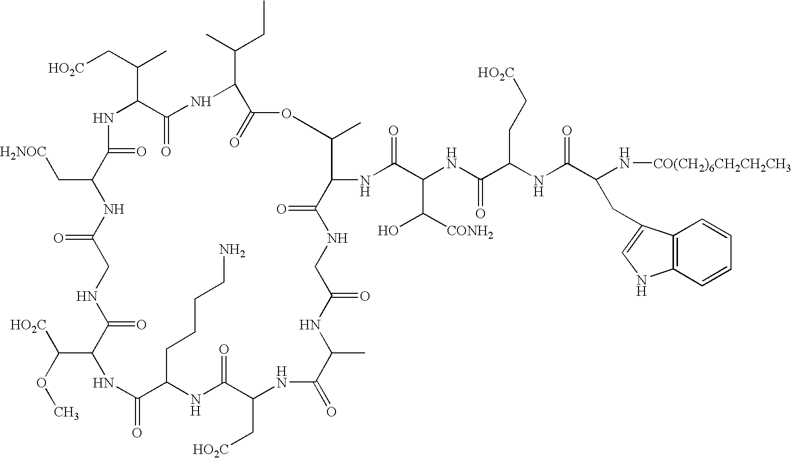 |
| C182 | 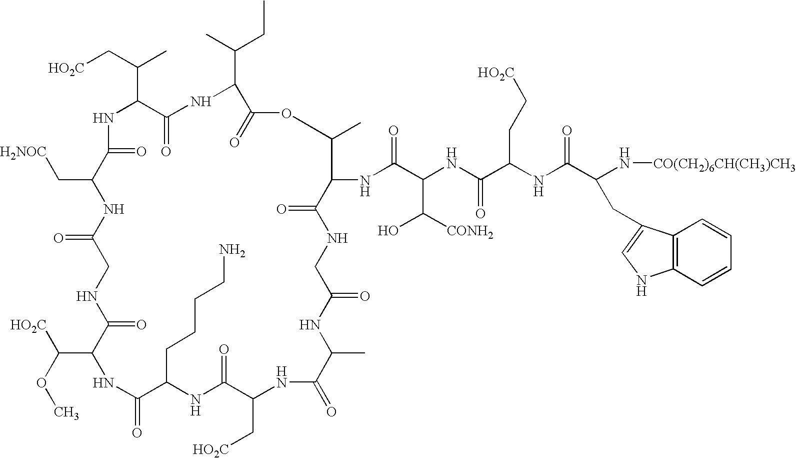 |
| C183 | 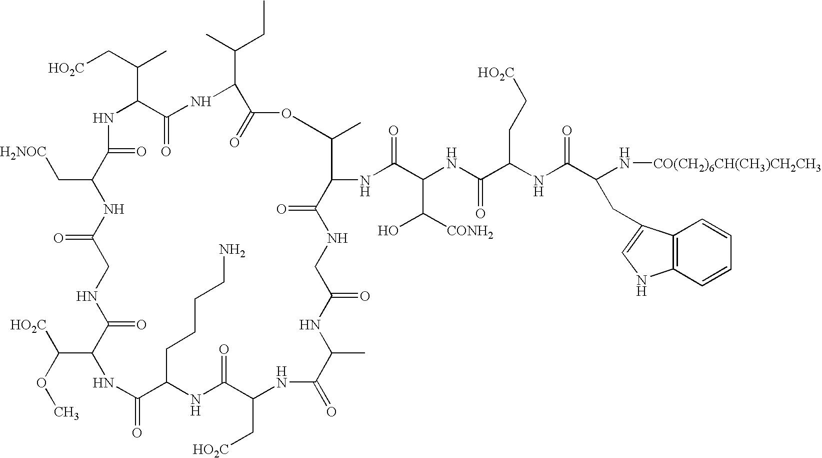 |
| C184 | 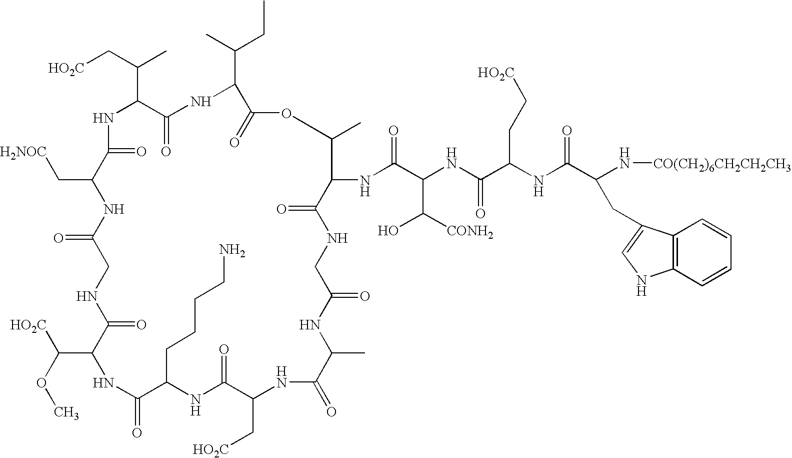 |
| C185 | 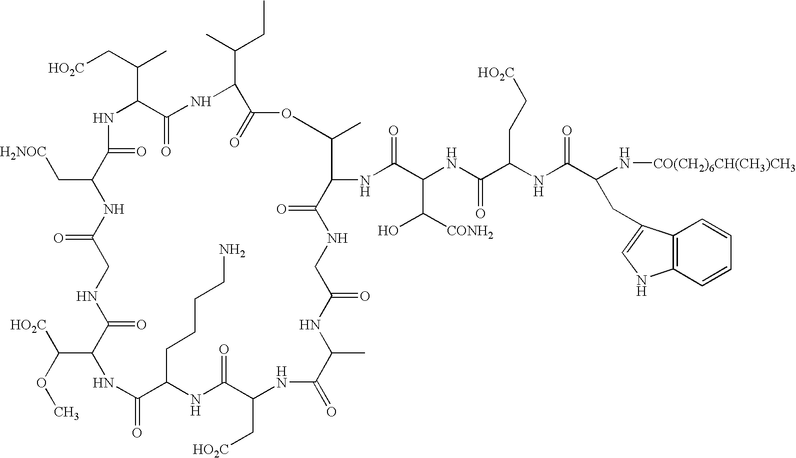 |
| C189 | 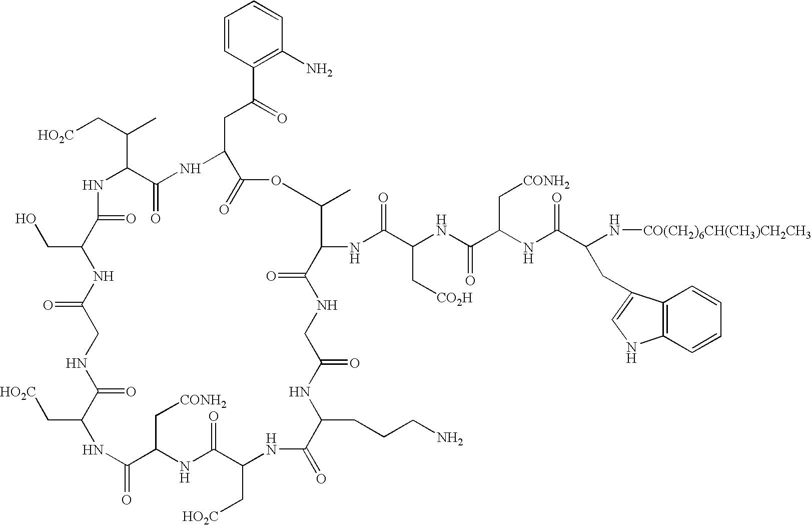 |
| C190 | 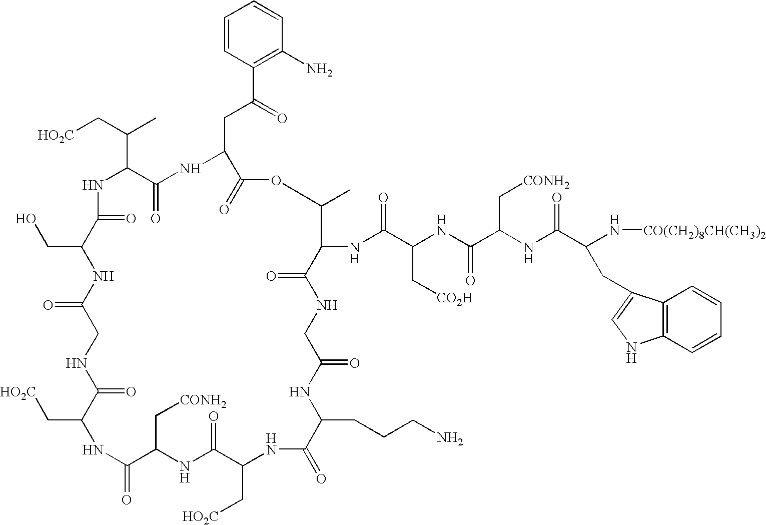 |
| C191 | 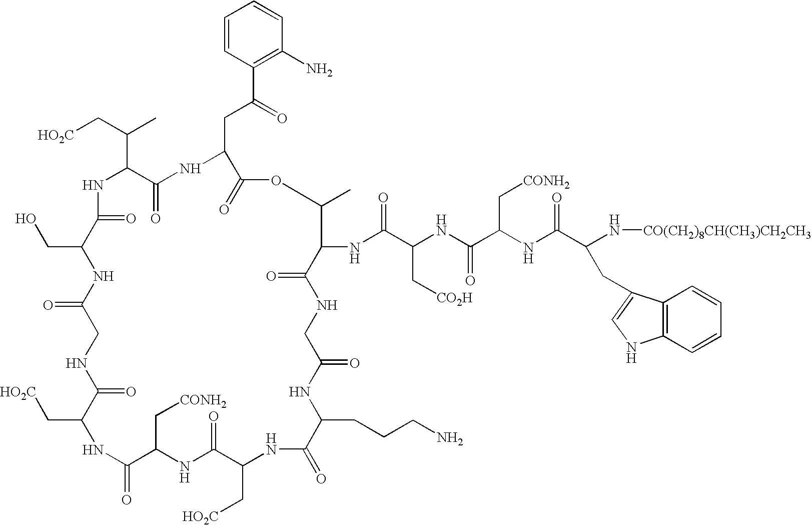 |
| C192 | 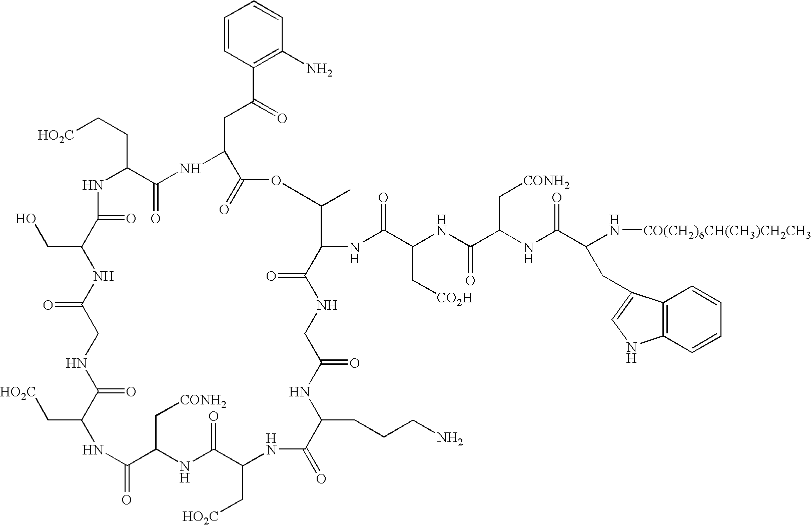 |
| C193 | 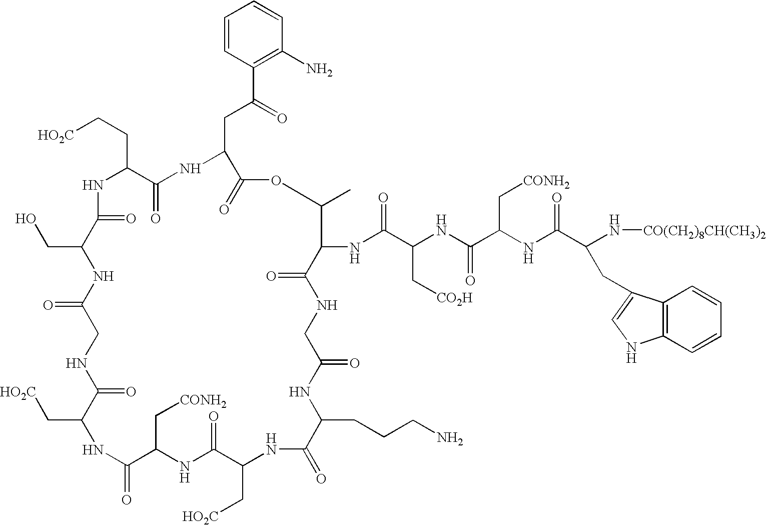 |
| C194 | 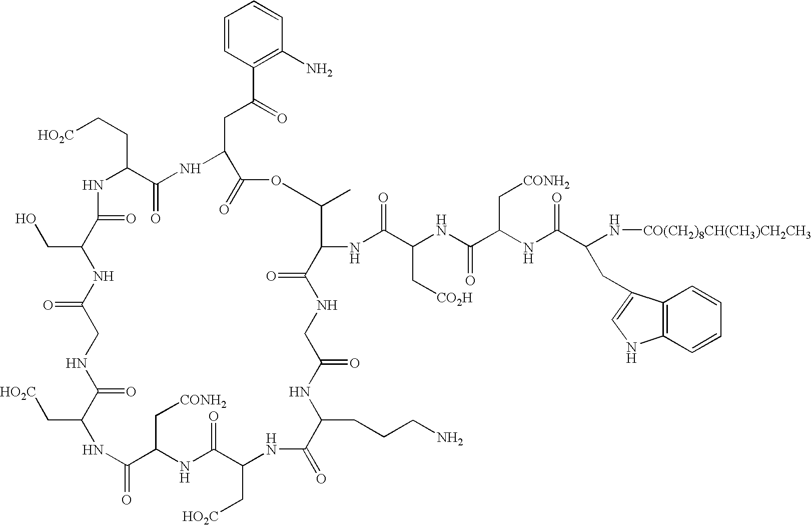 |
| C195 | 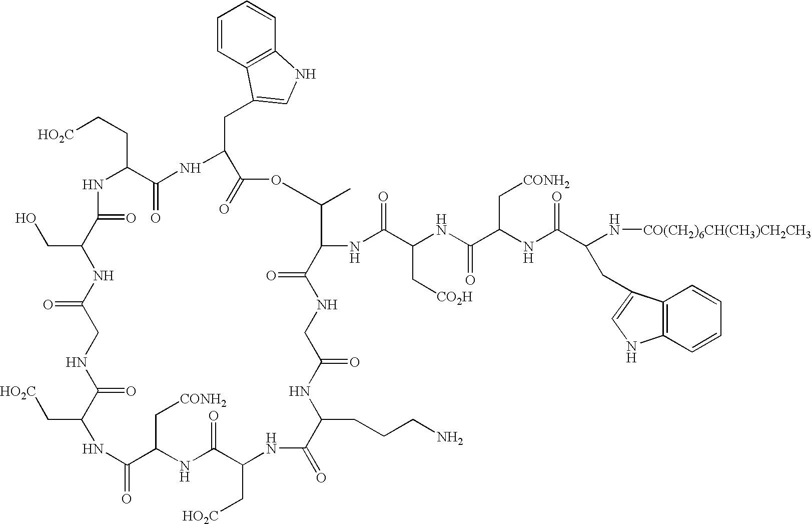 |
| C196 | 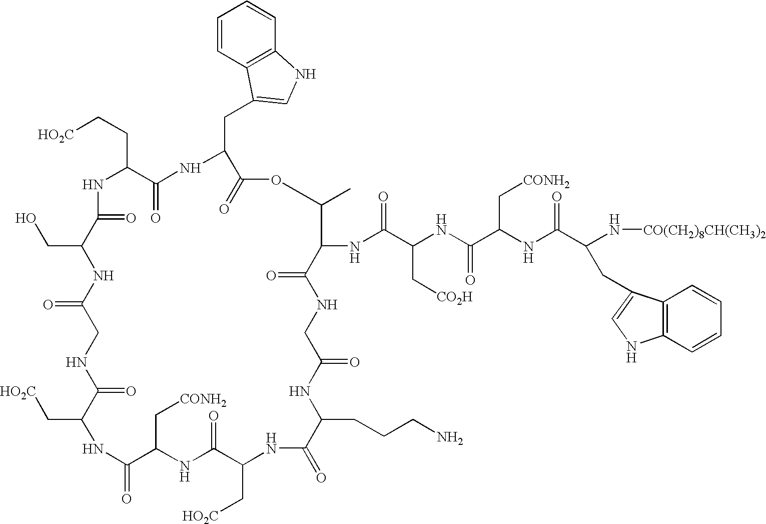 |
| C197 | 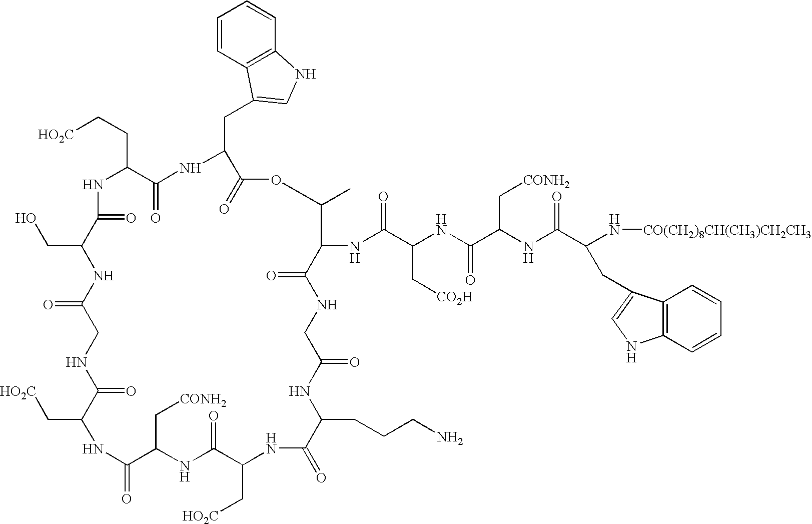 |
| C198 | 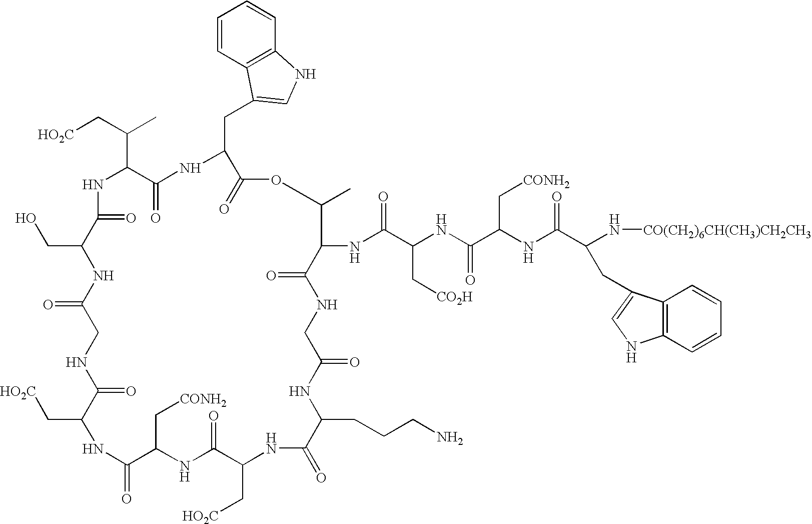 |
| C199 | 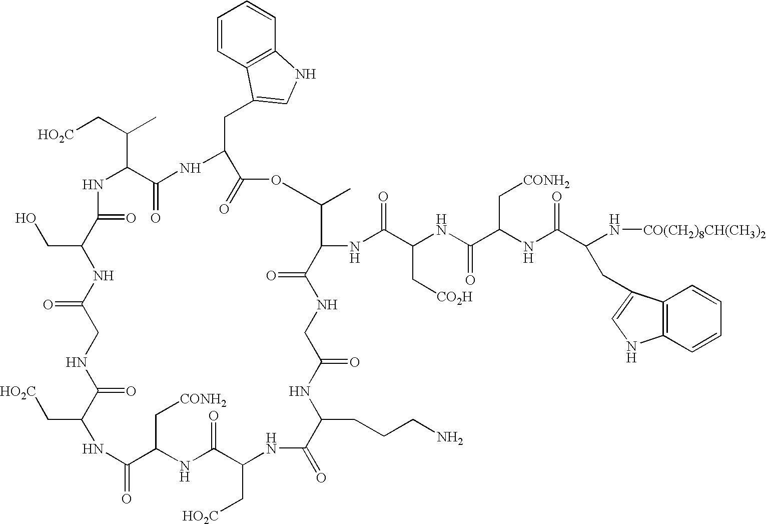 |
| C200 | 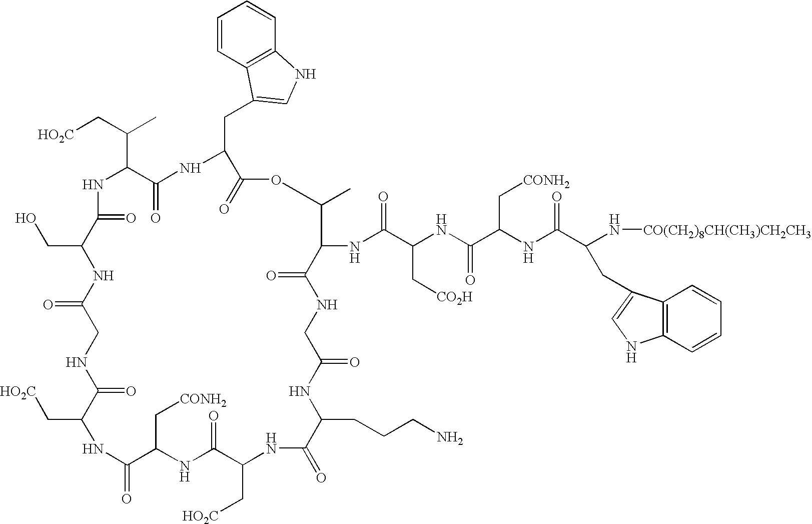 |
| C201 | 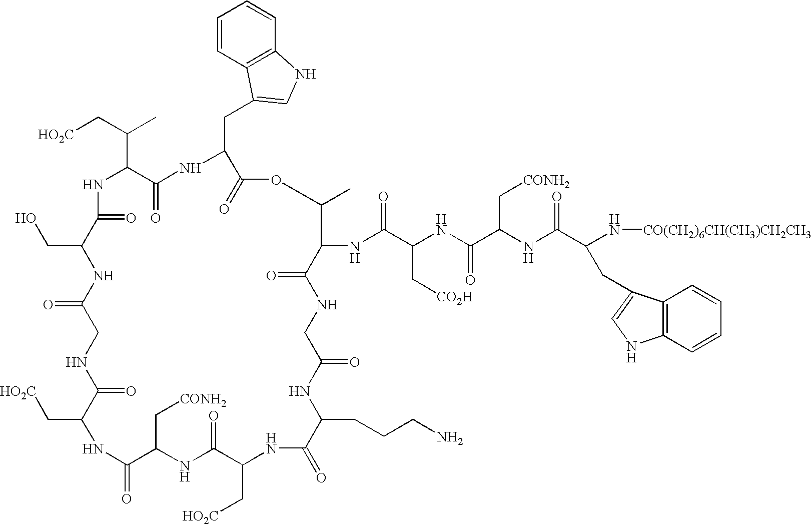 |
| C202 | 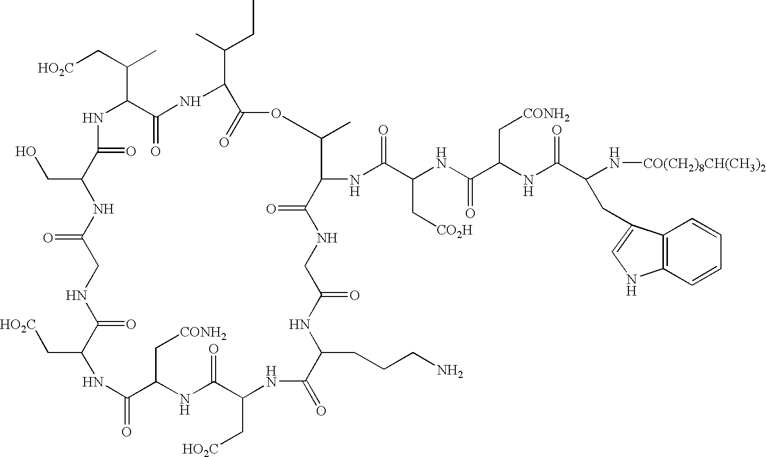 |
| C203 | 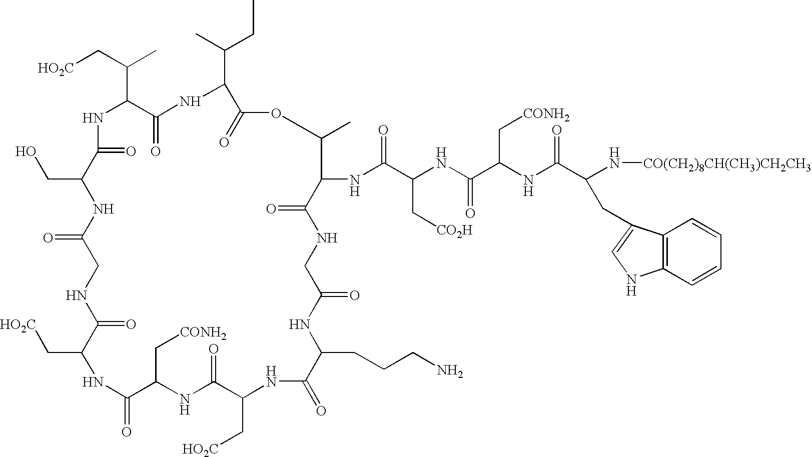 |
| C204 | 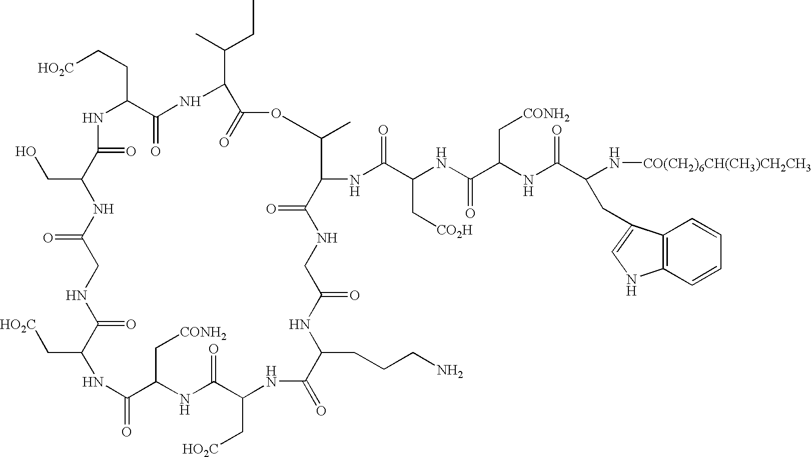 |
| C205 | 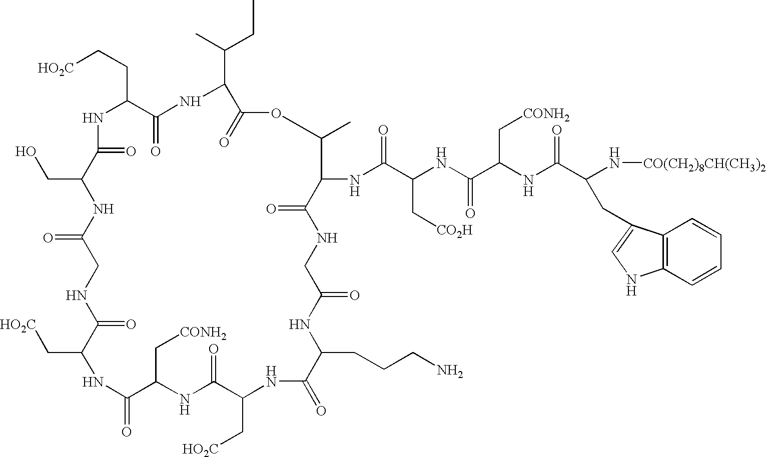 |
| C206 | 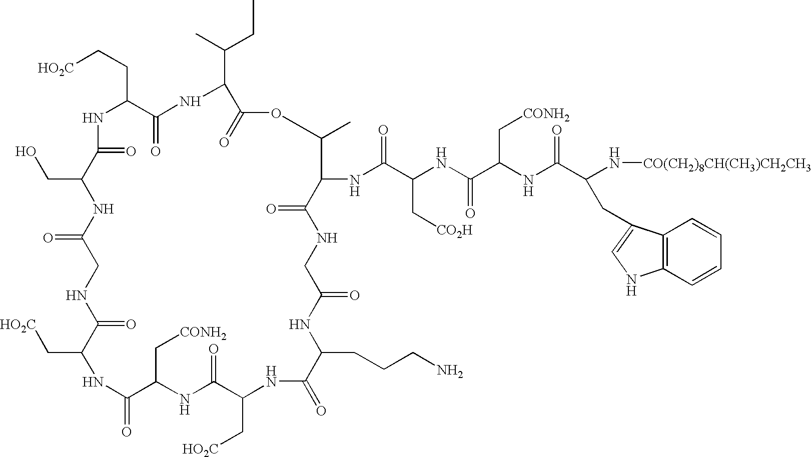 |
| C207 | 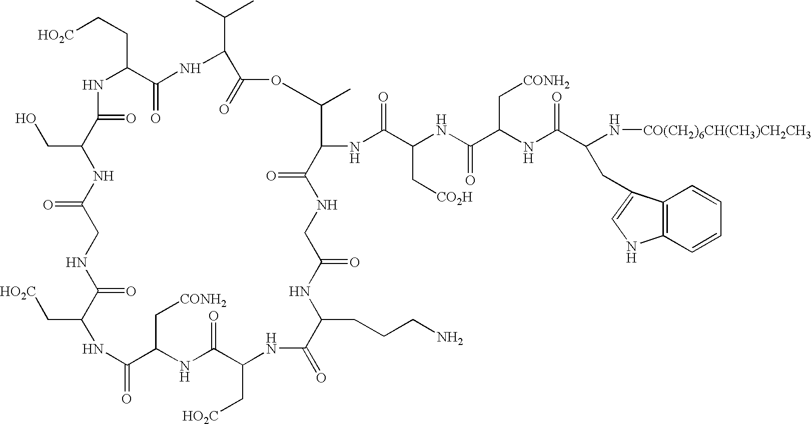 |
| C208 | 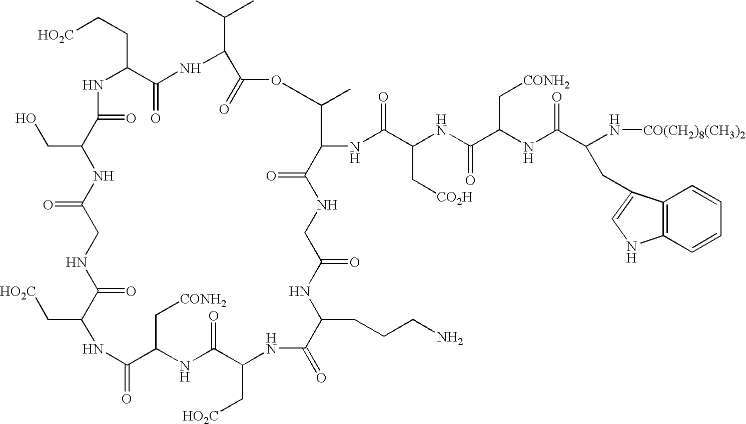 |
| C209 | 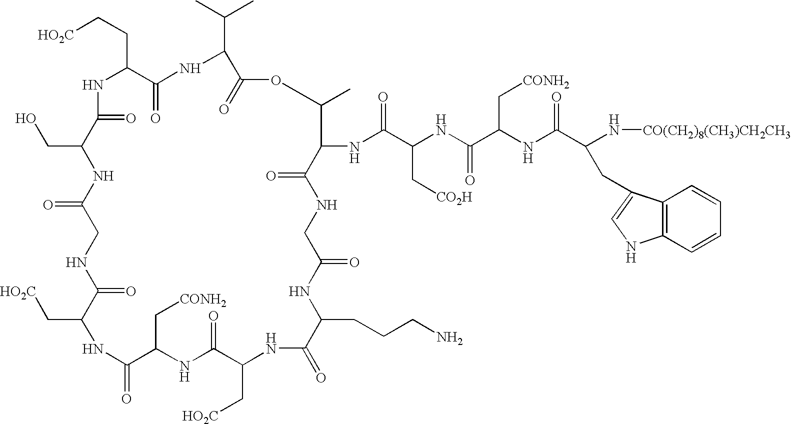 |
| C210 | 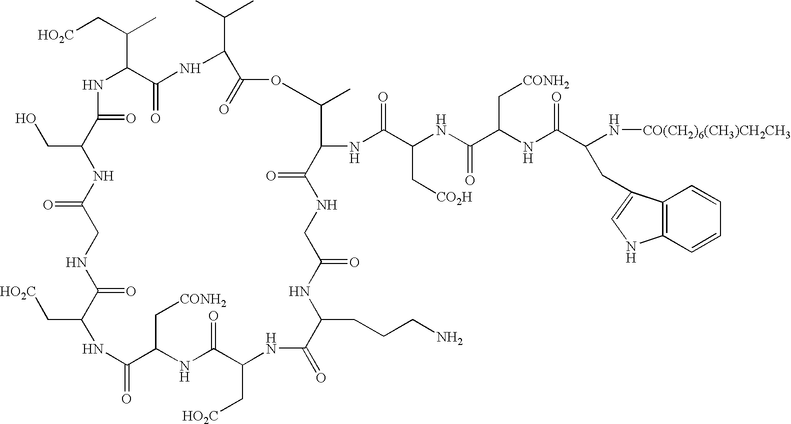 |
| C211 | 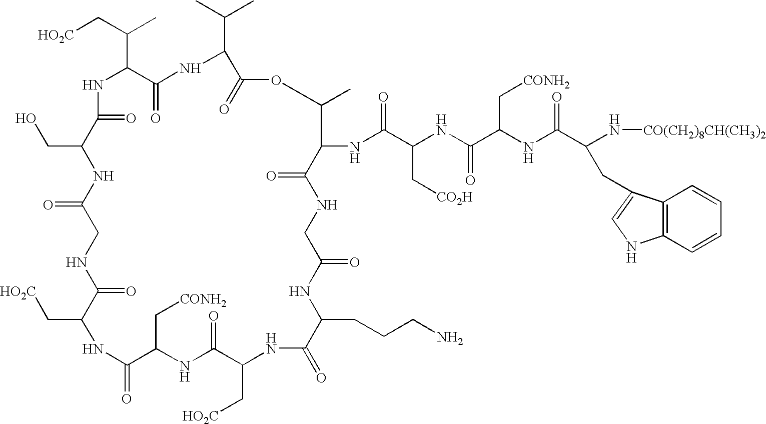 |
| C212 | 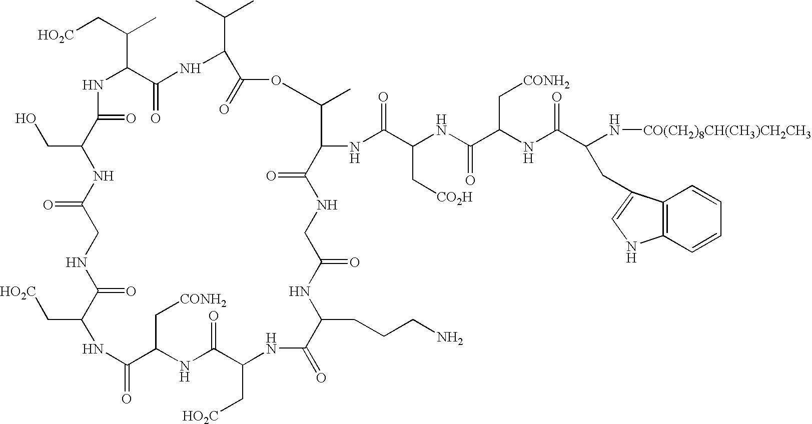 |
| C213 | 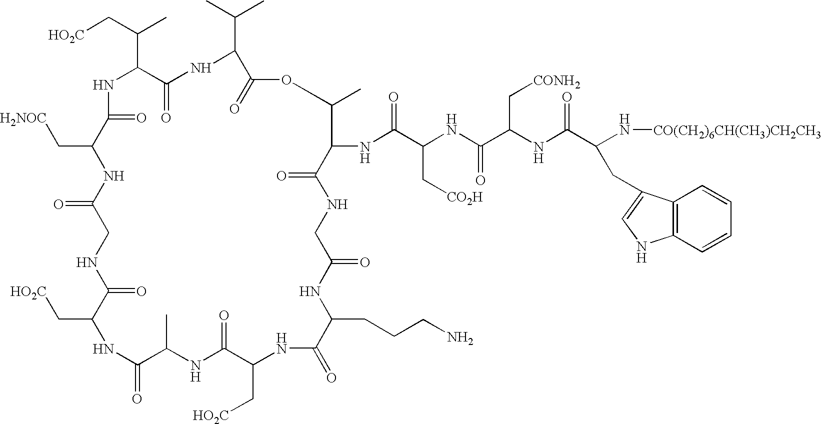 |
| C214 | 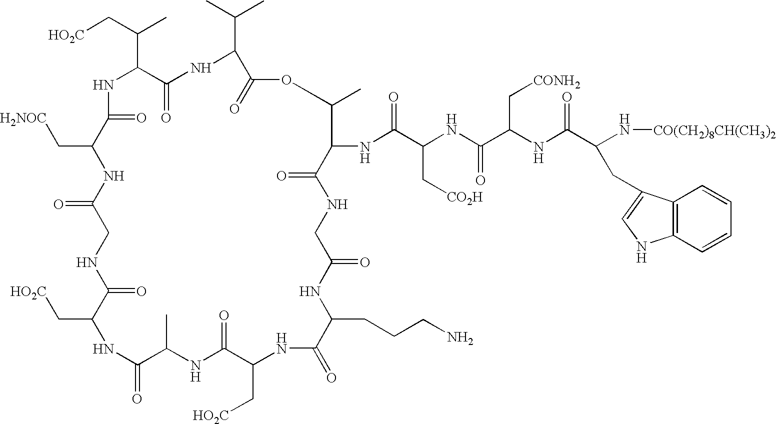 |
| C215 | 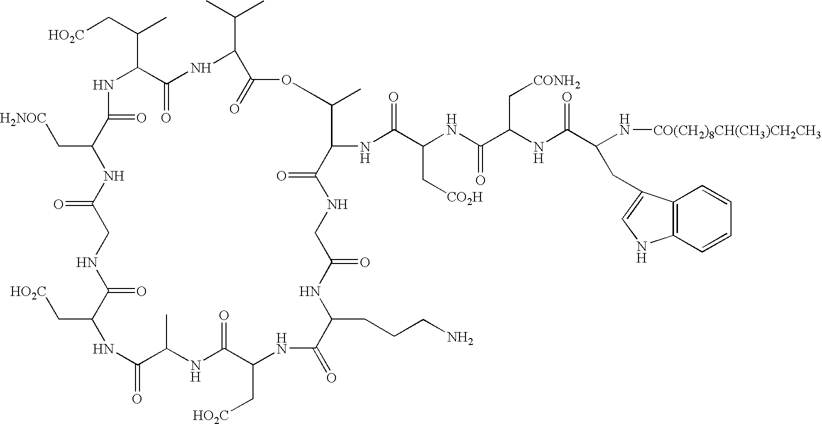 |
| C216 | 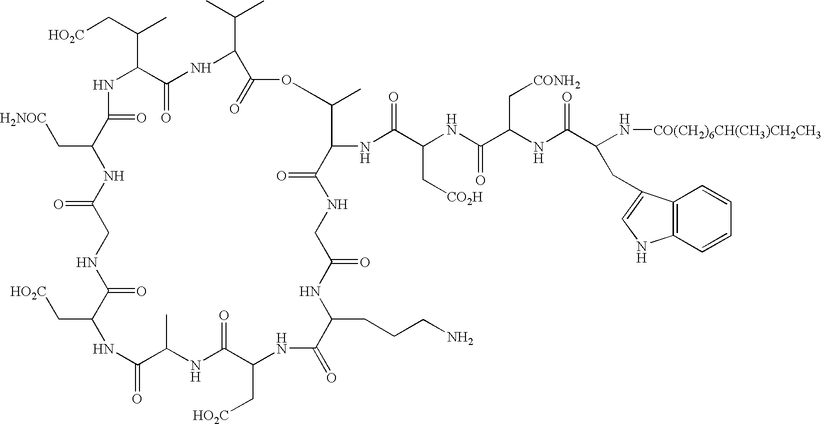 |
| C217 | 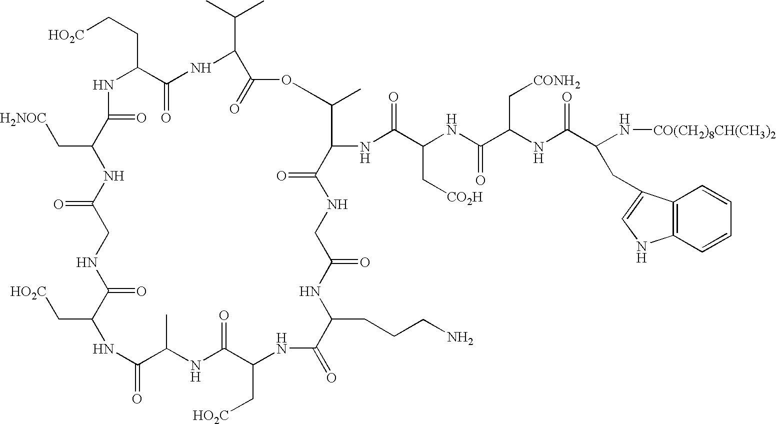 |
| C218 | 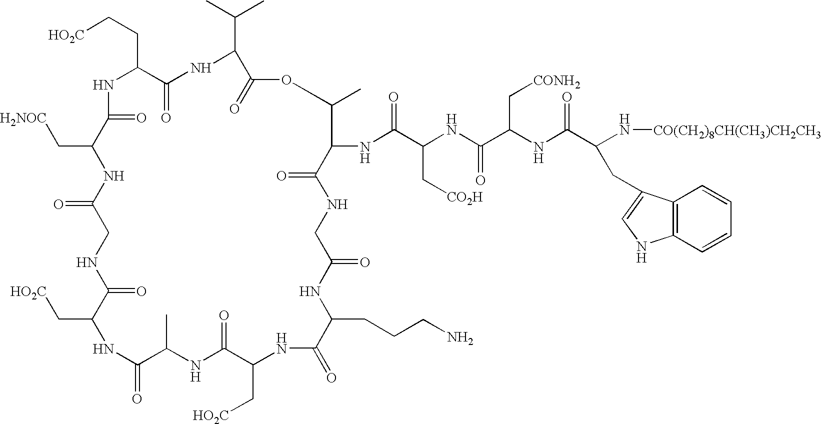 |
| C219 | 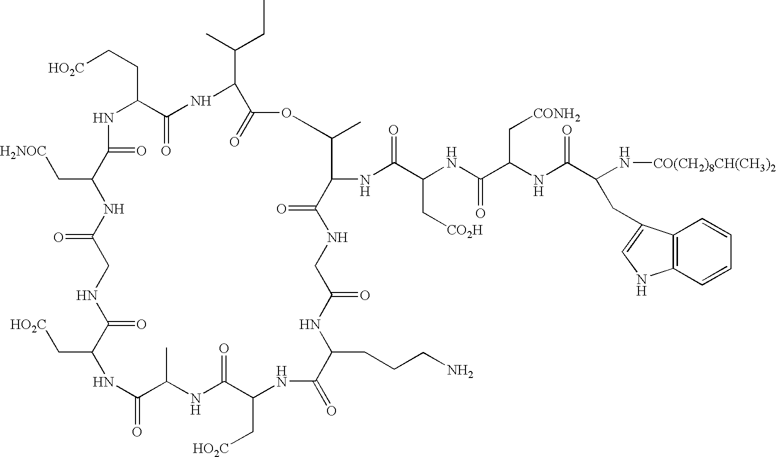 |
| C220 | 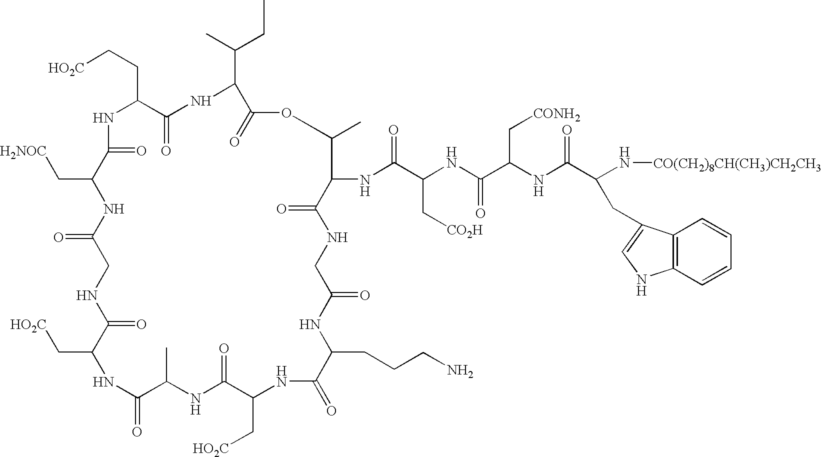 |
| C221 | 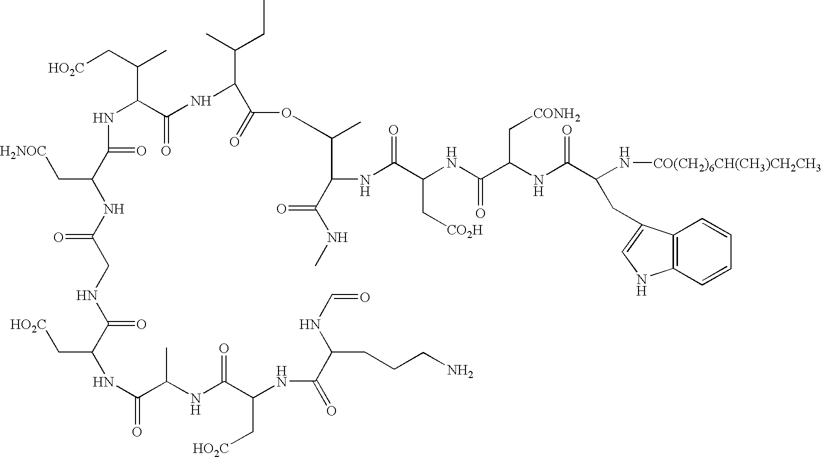 |
| C222 | 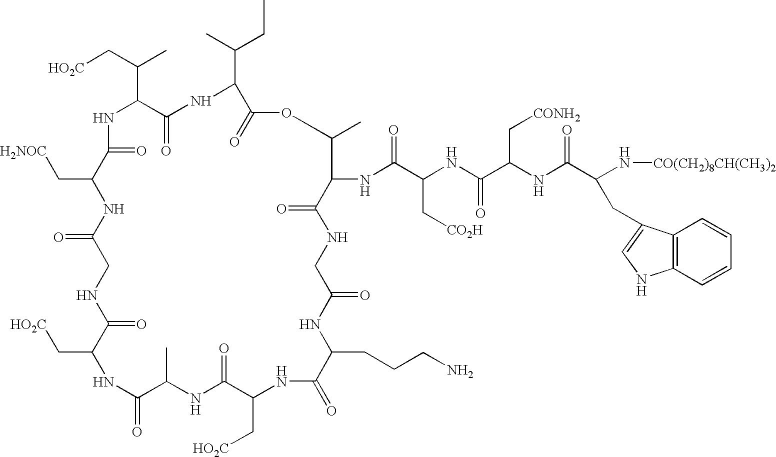 |
| C223 | 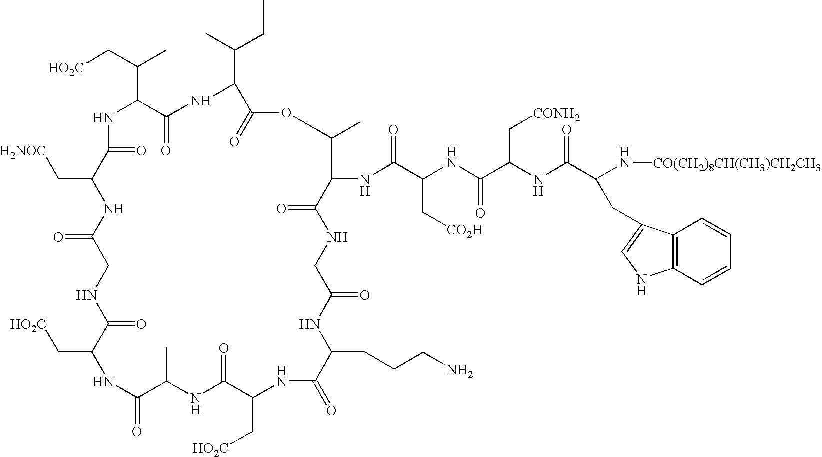 |
| C224 | 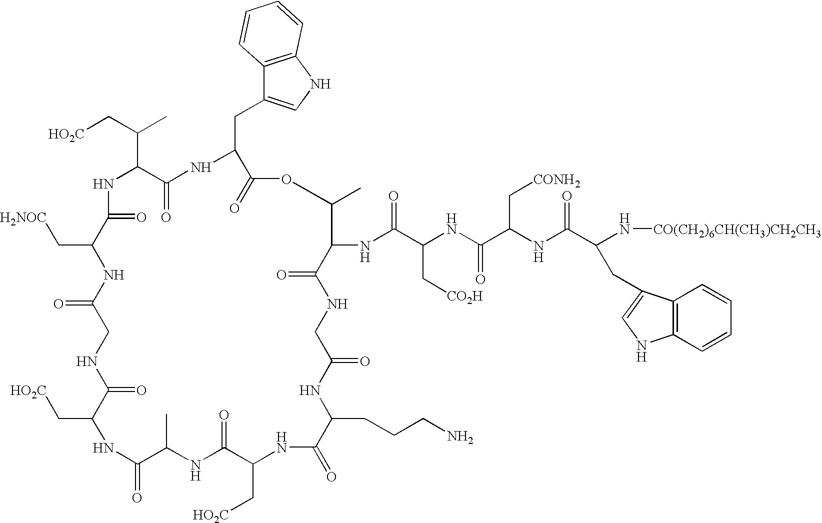 |
| C225 | 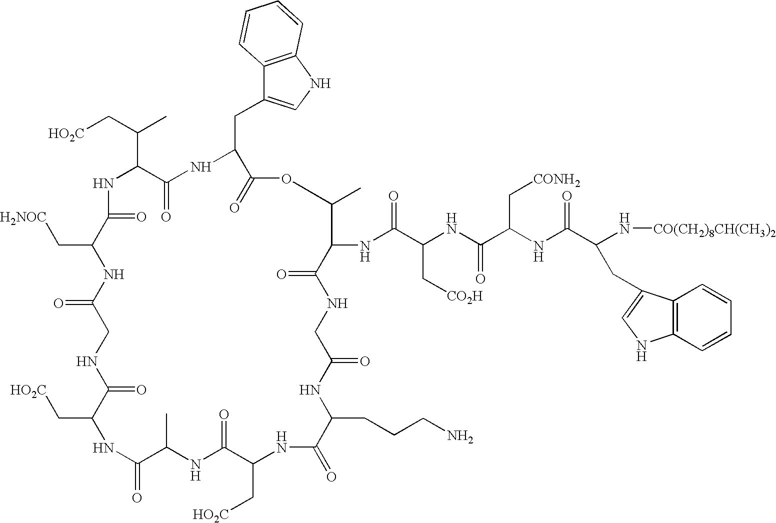 |
| C226 | 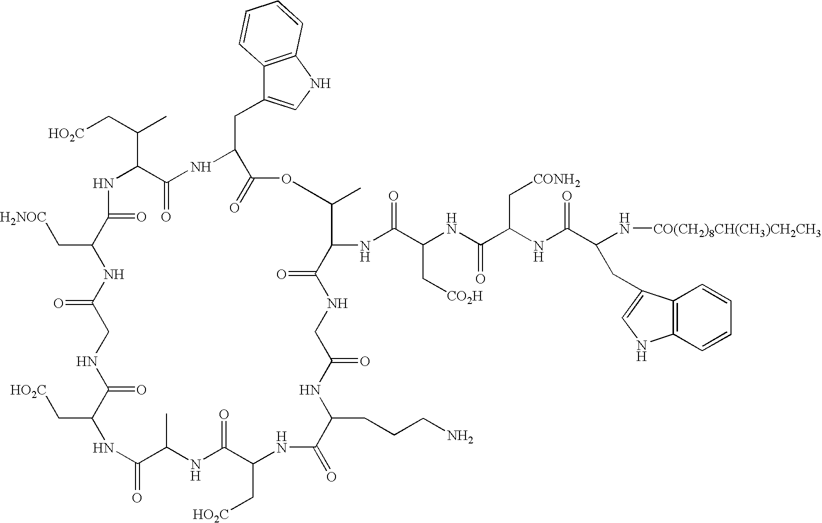 |
| C227 | 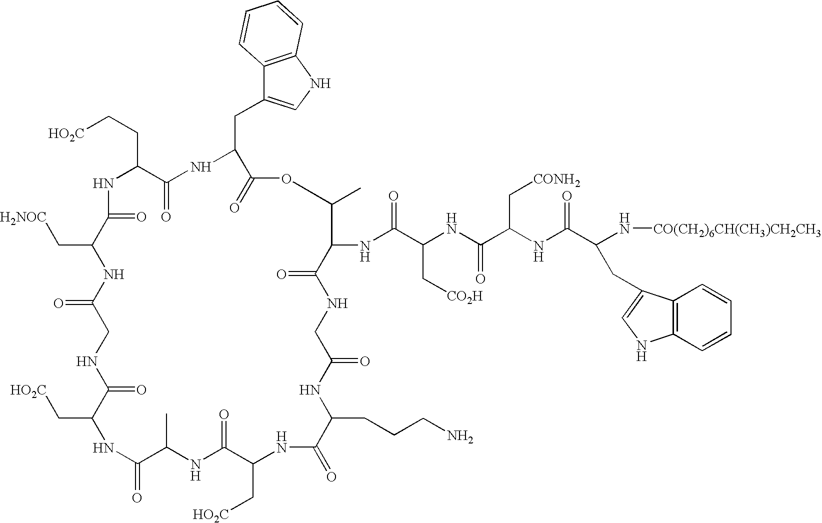 |
| C228 | 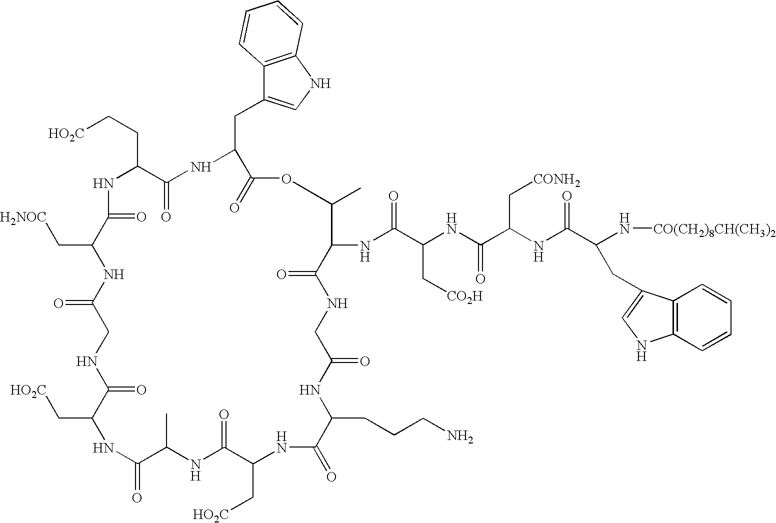 |
| C229 | 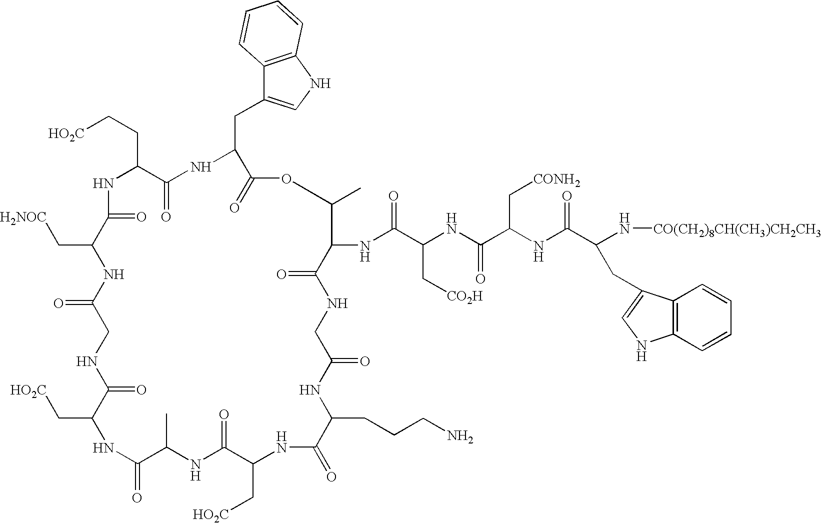 |
| C230 | 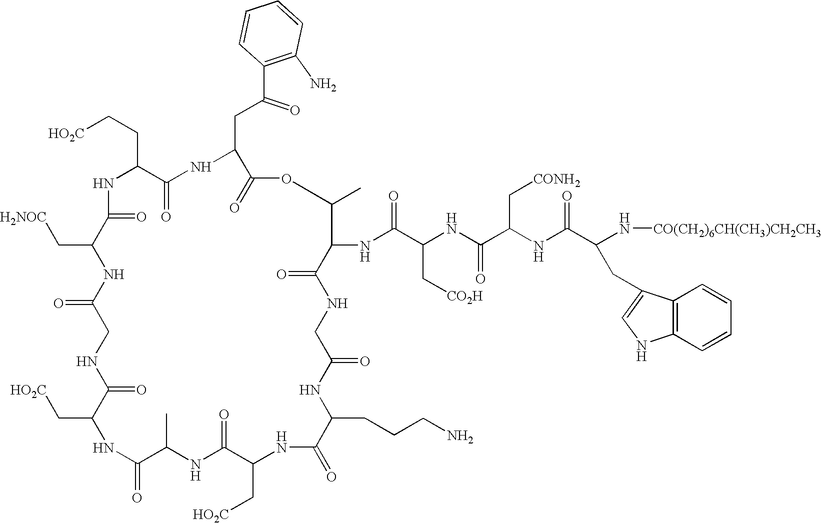 |
| C231 | 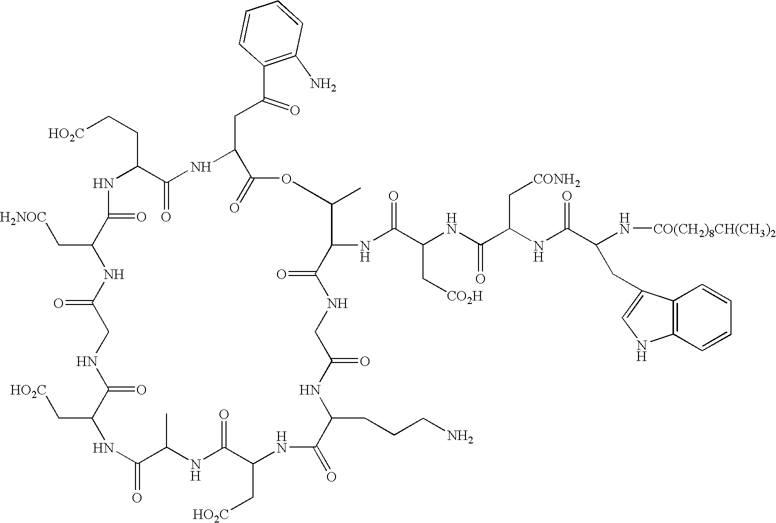 |
| C232 | 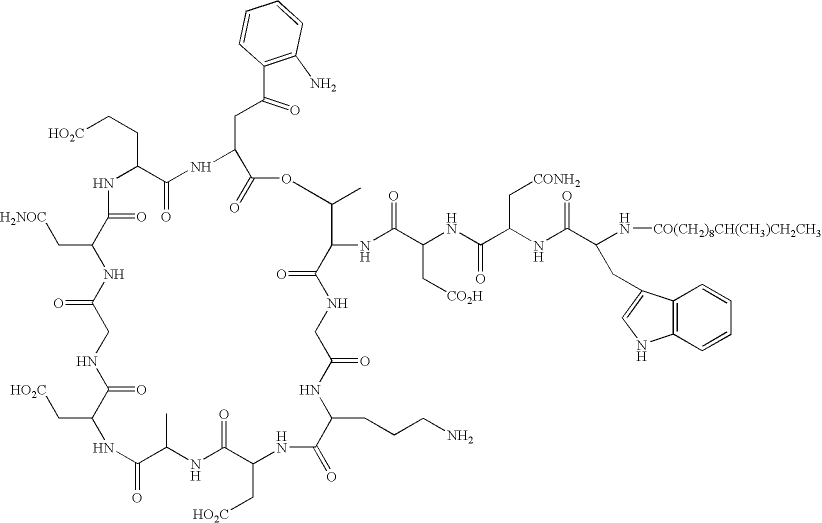 |
| C233 | 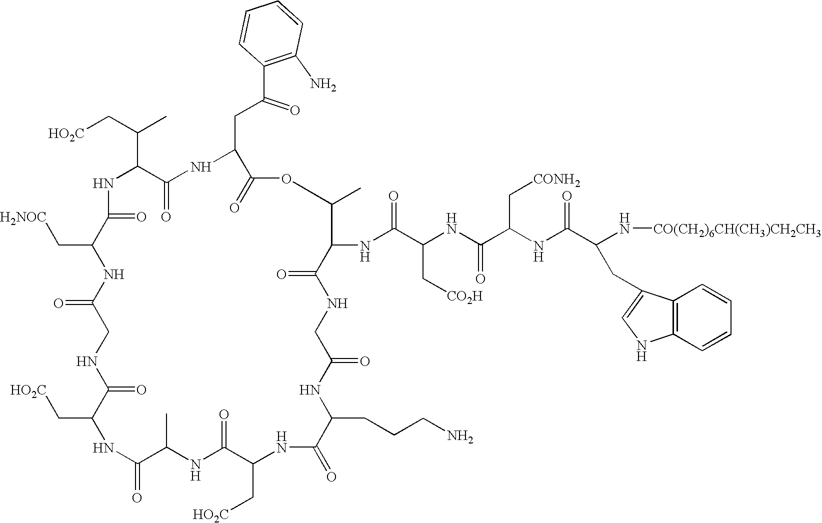 |
| C234 | 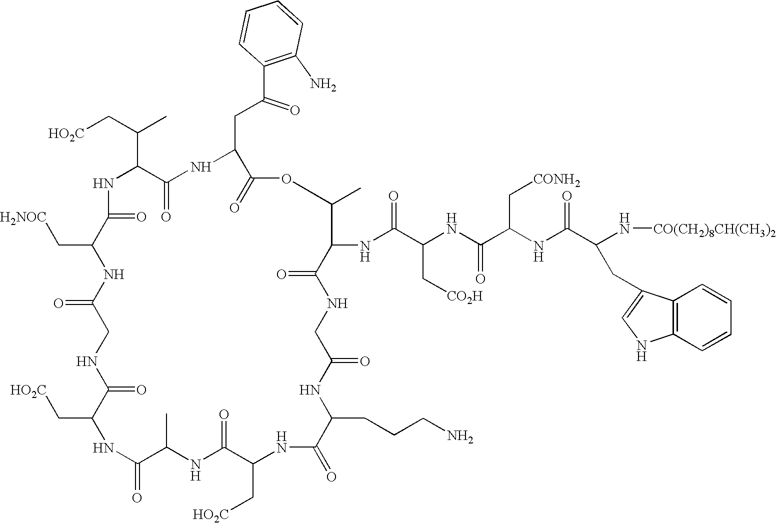 |
| C235 | 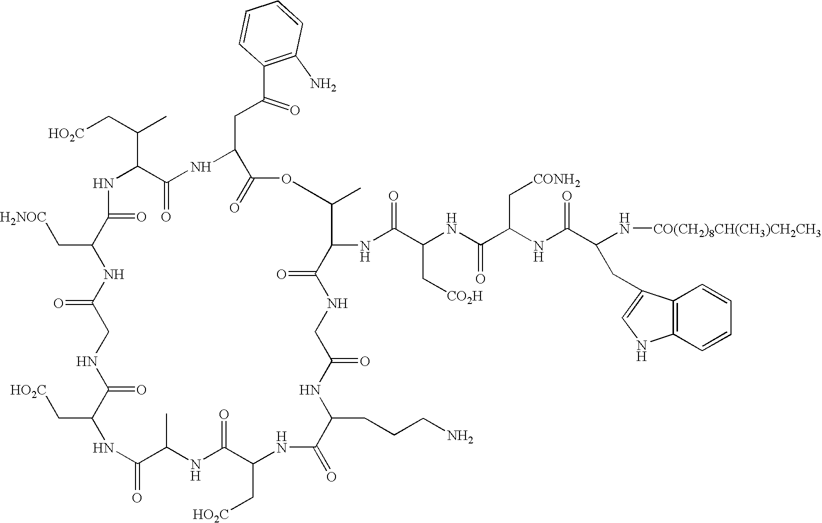 |
| C236 | 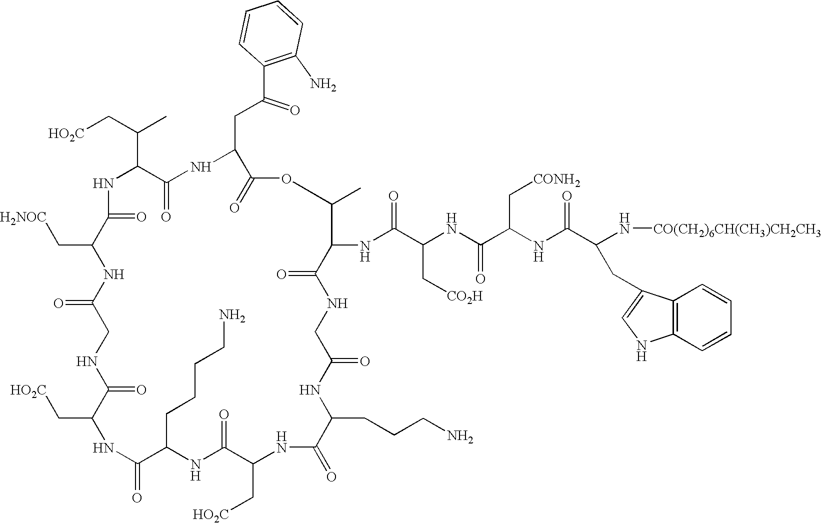 |
| C237 | 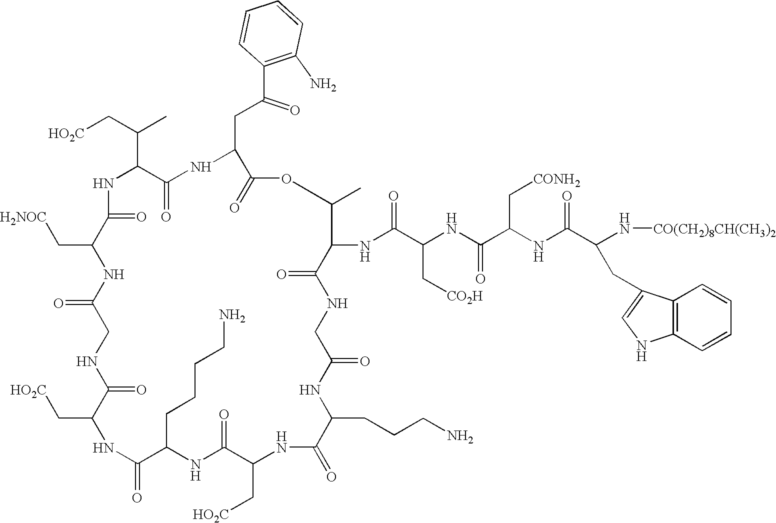 |
| C238 | 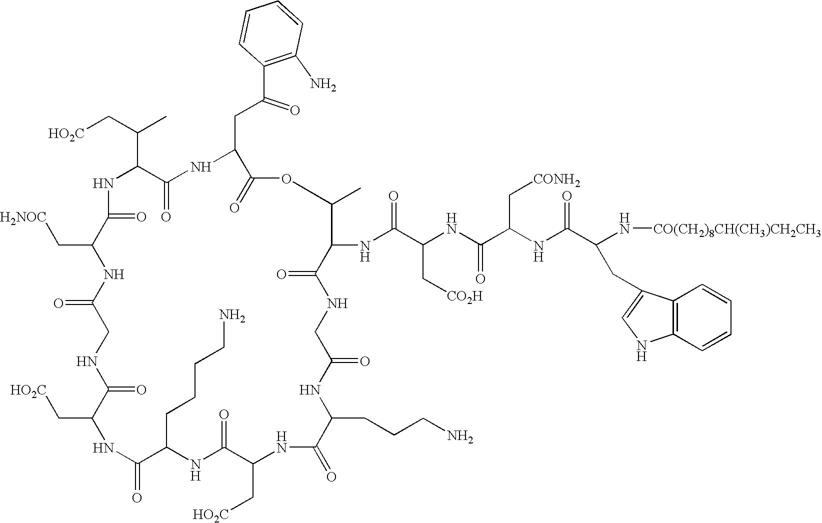 |
| C259 | 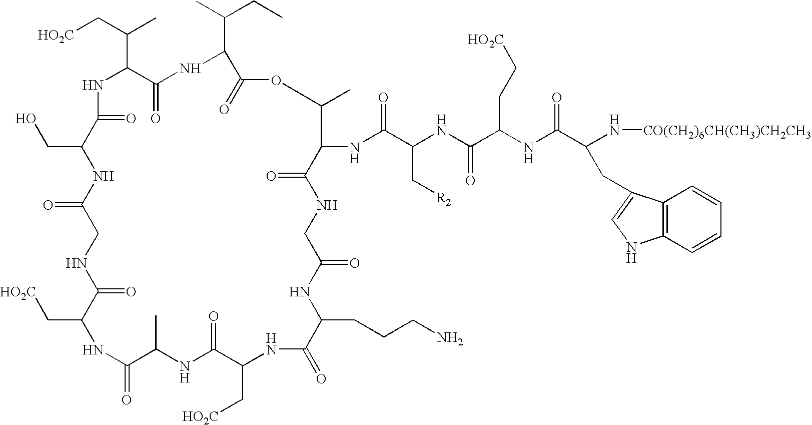 |
| C260 | 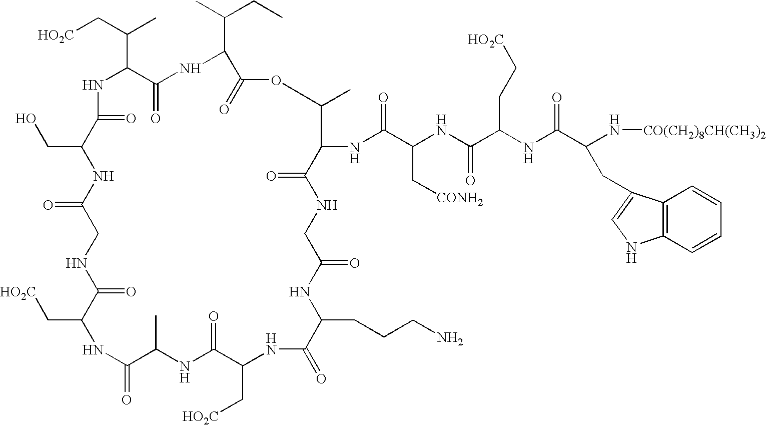 |
| C261 | 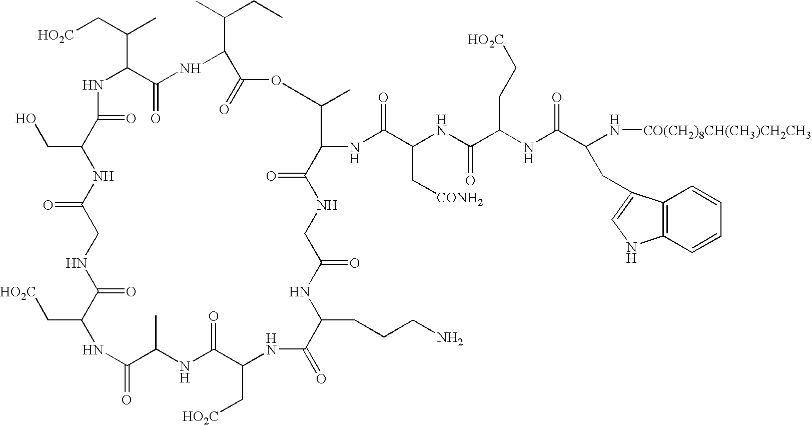 |
| C262 | 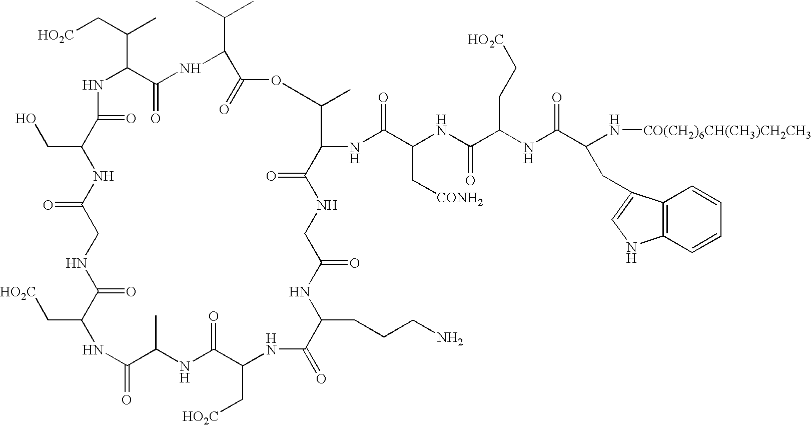 |
| C263 | 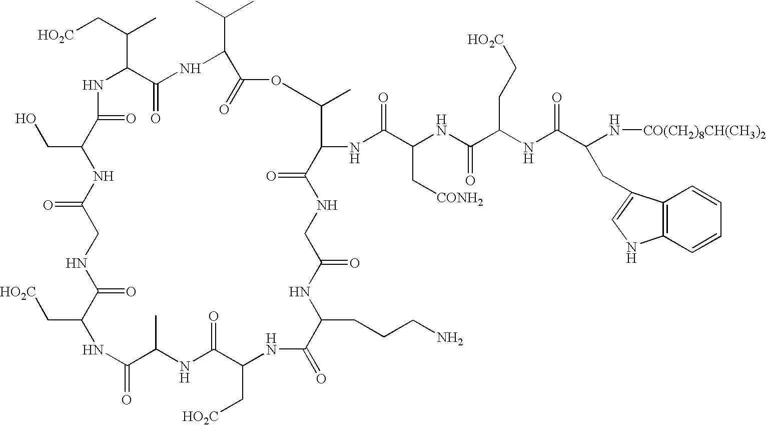 |
| C264 | 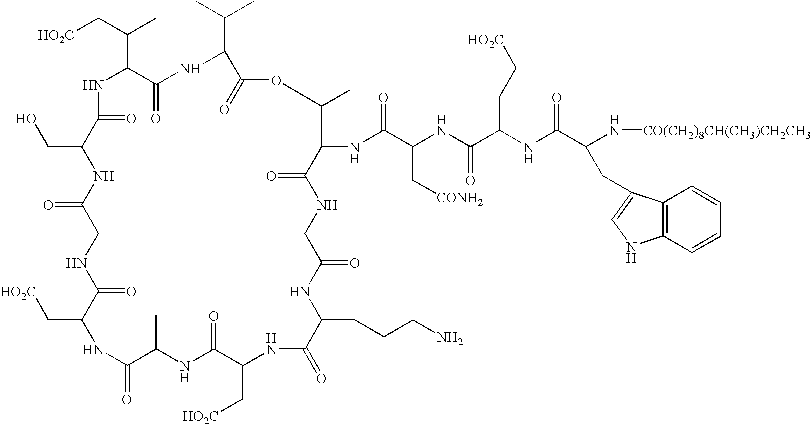 |
| C265 | 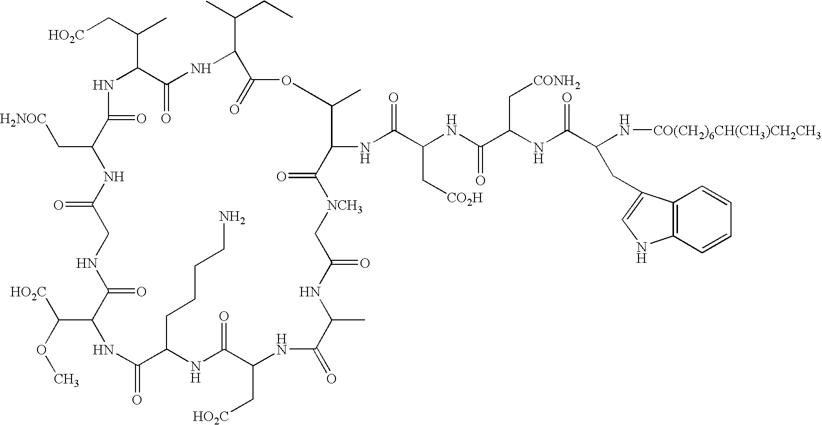 |
| C266 | 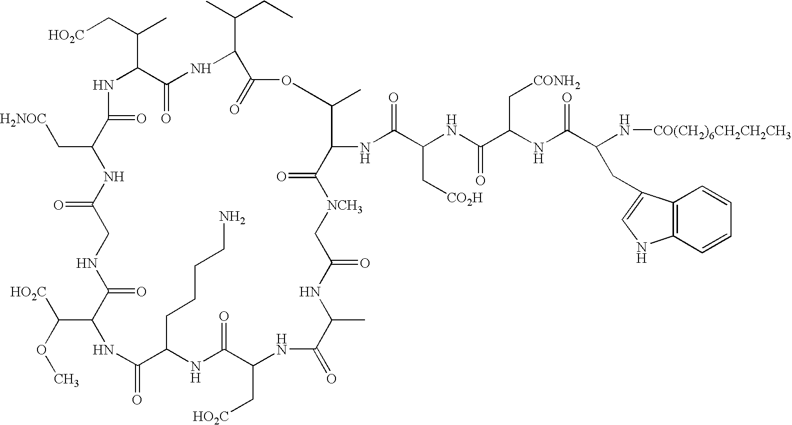 |
| C267 | 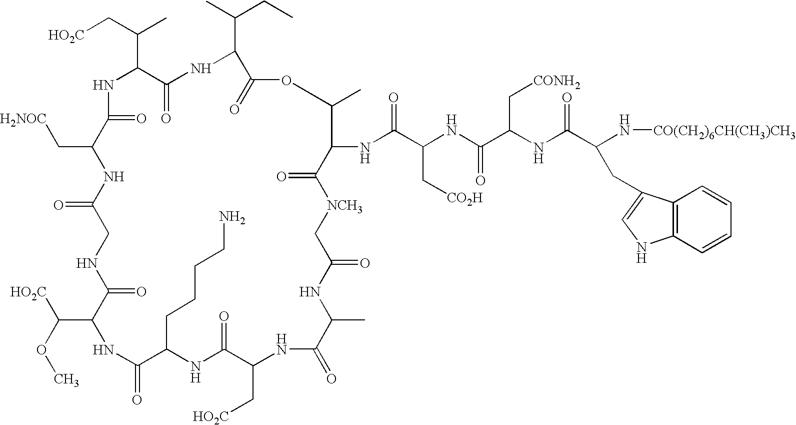 |
| C268 | 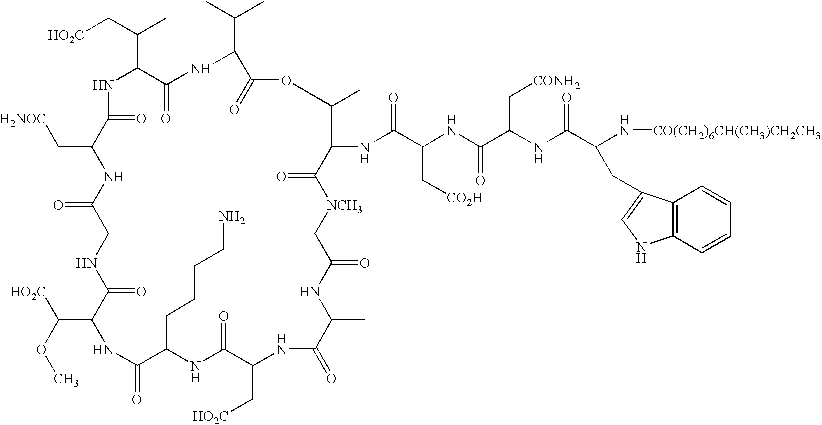 |
| C269 | 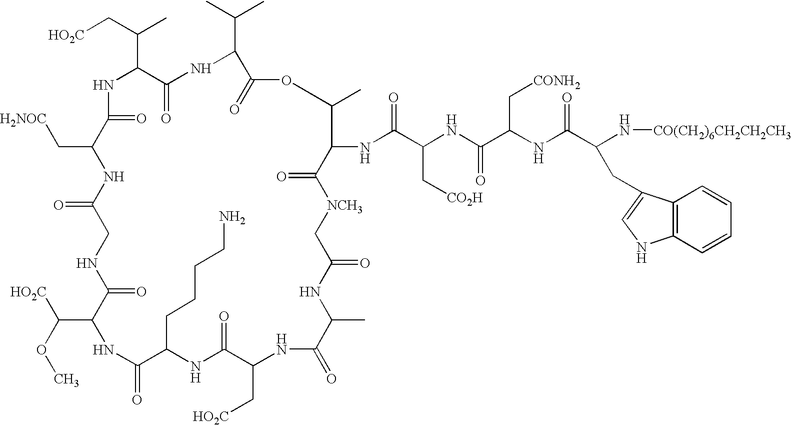 |
| C270 | 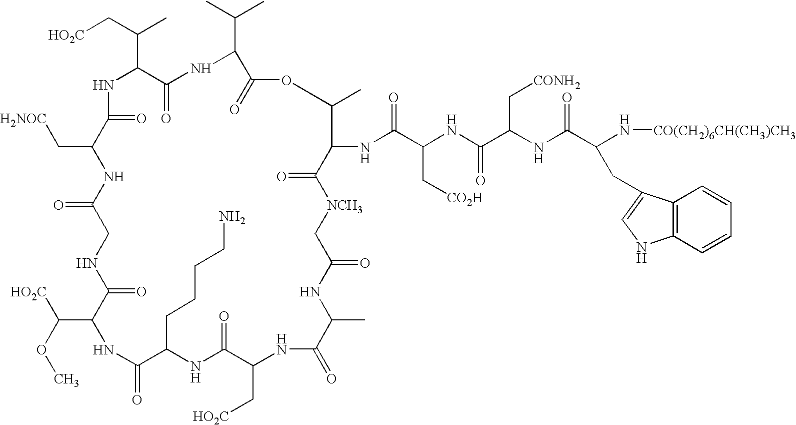 |
| C271 | 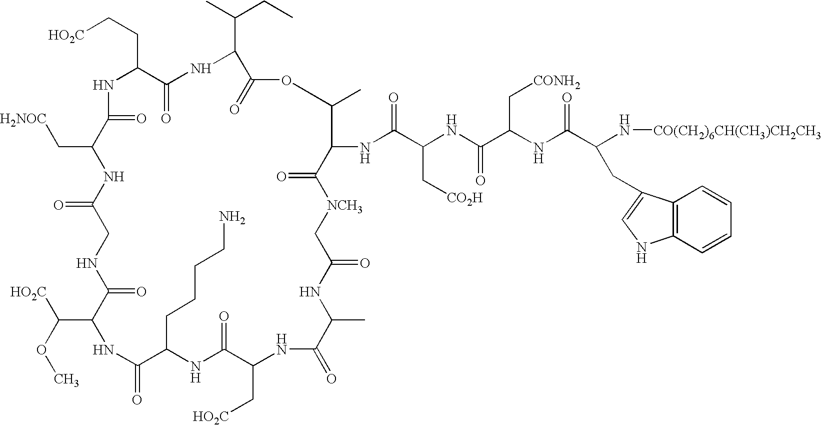 |
| C272 | 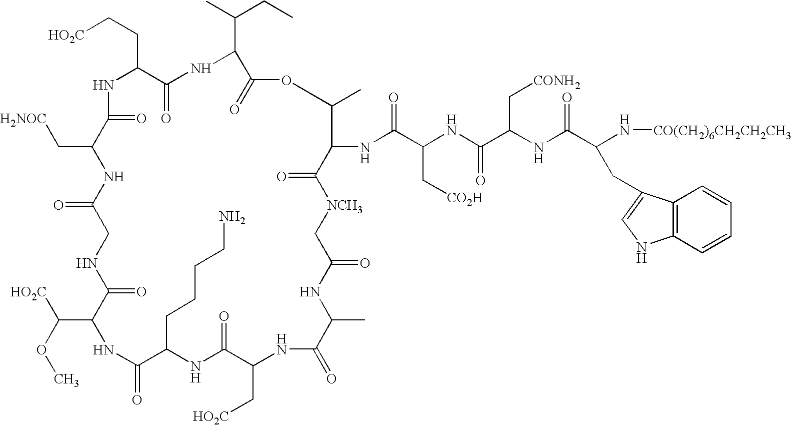 |
| C273 | 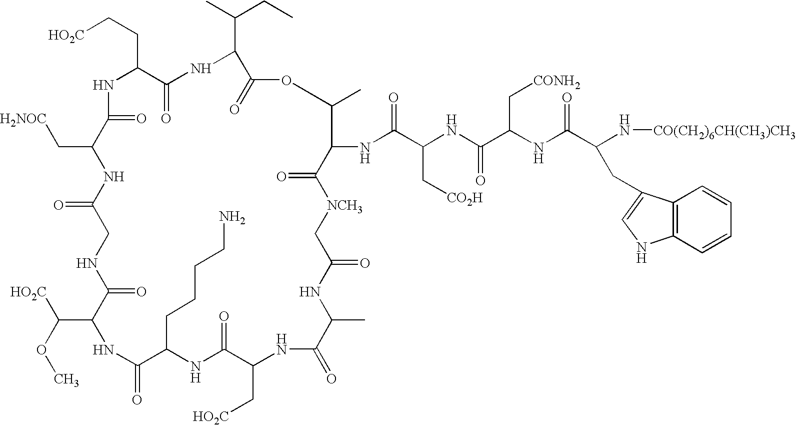 |
| C274 | 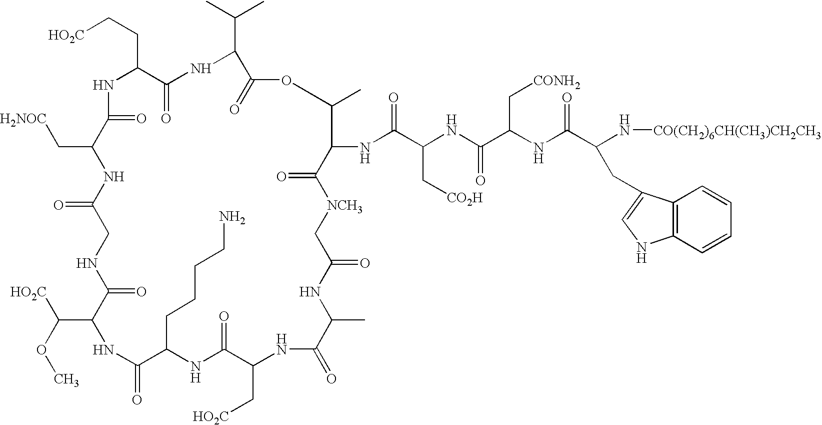 |
| C275 | 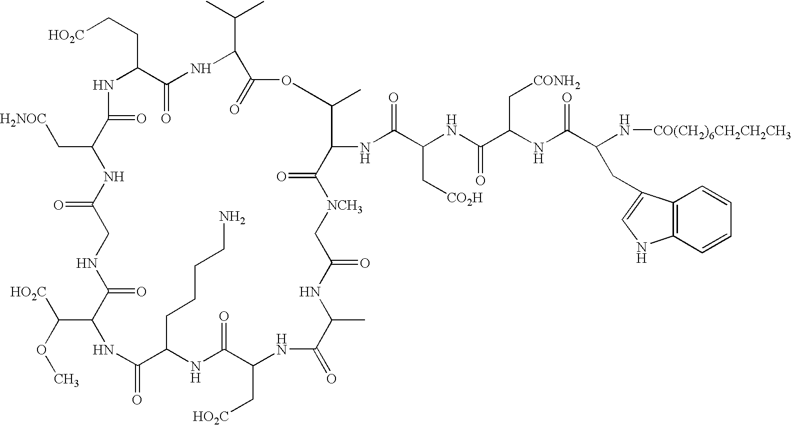 |
| C276 | 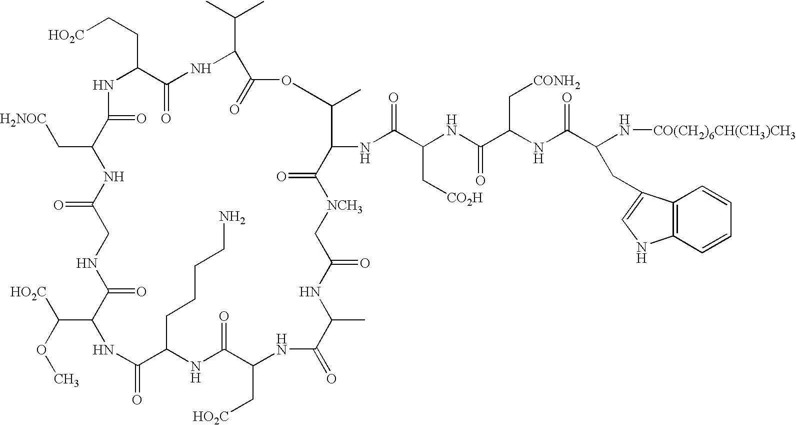 |
| C277 | 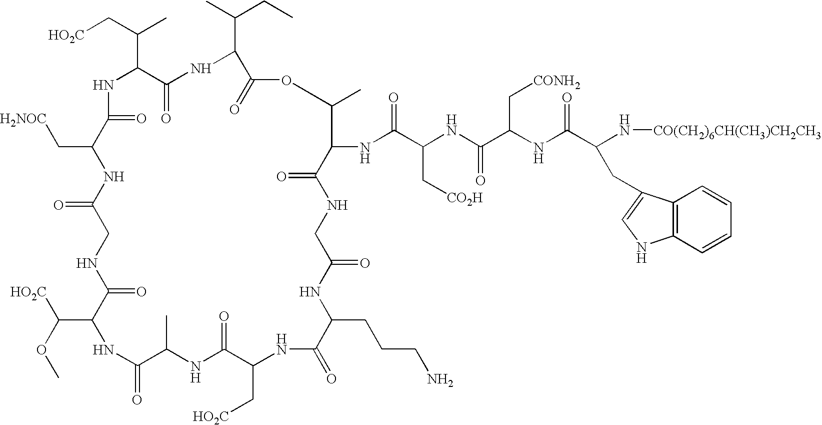 |
| C278 | 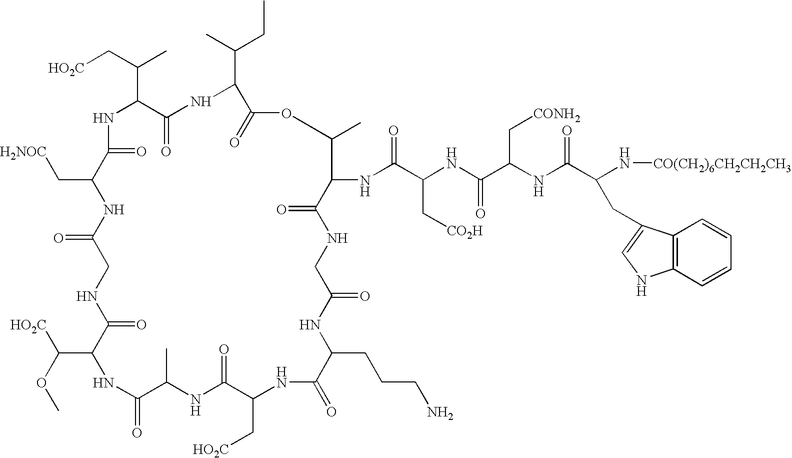 |
| C279 | 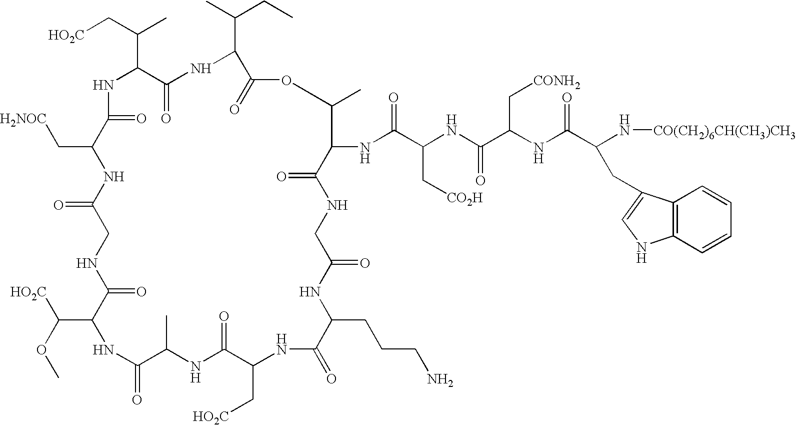 |
| C280 | 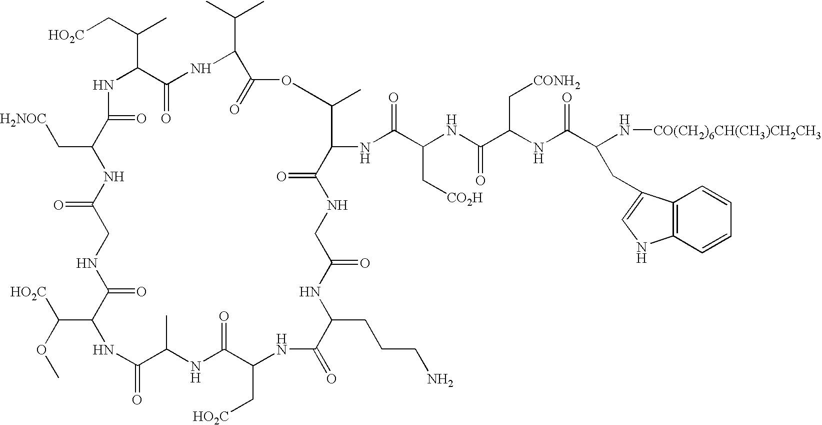 |
| C281 | 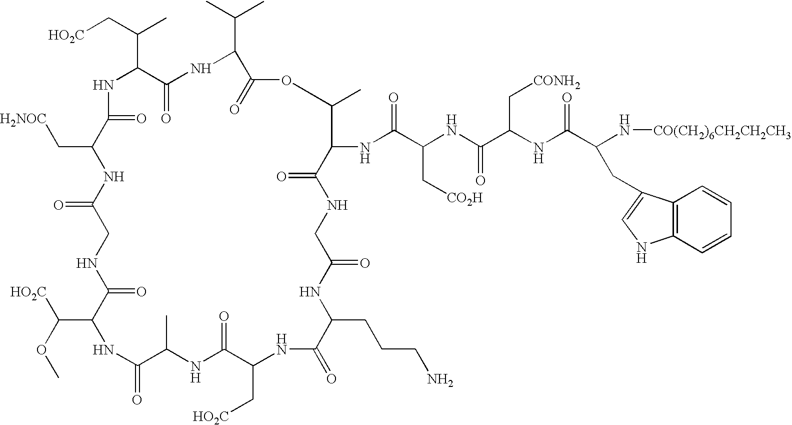 |
| C282 | 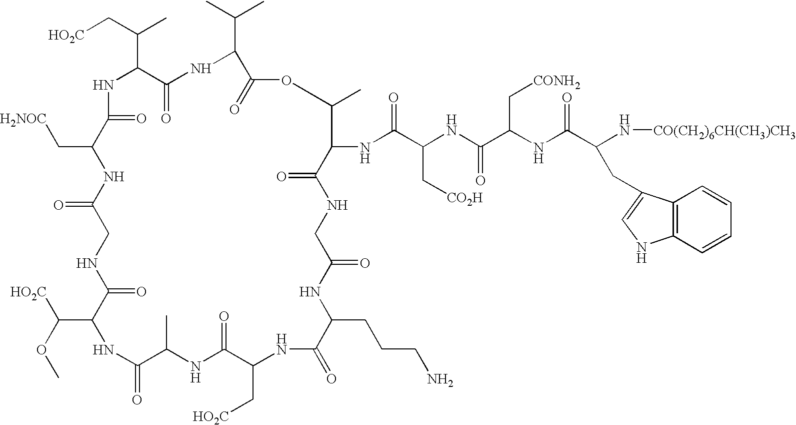 |
| C283 | 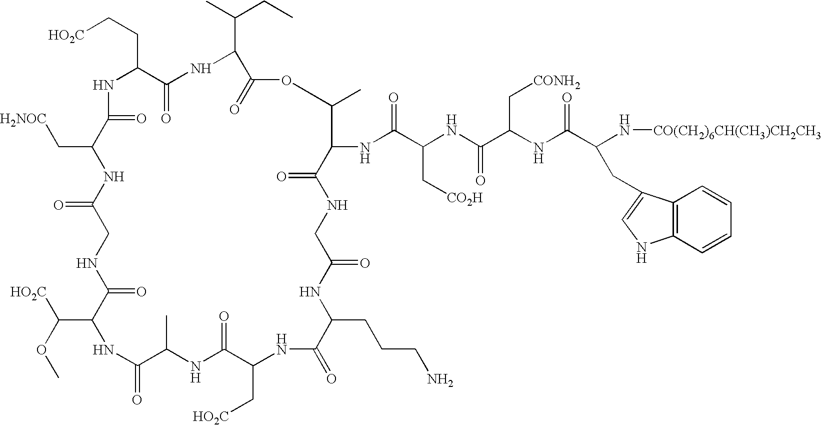 |
| C284 | 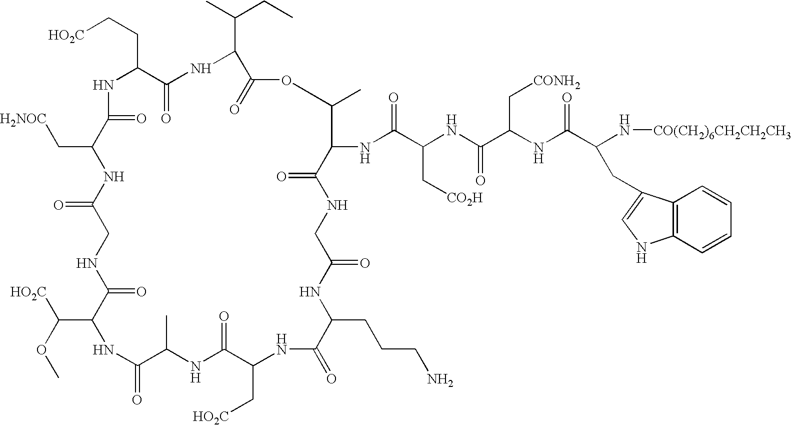 |
| C285 | 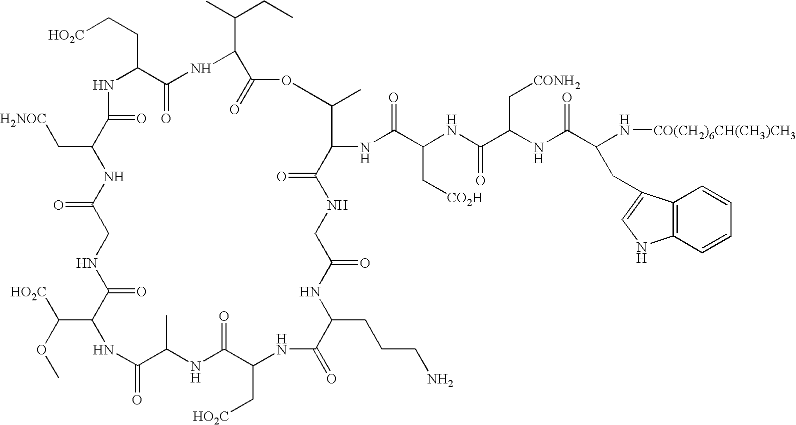 |
| C286 | 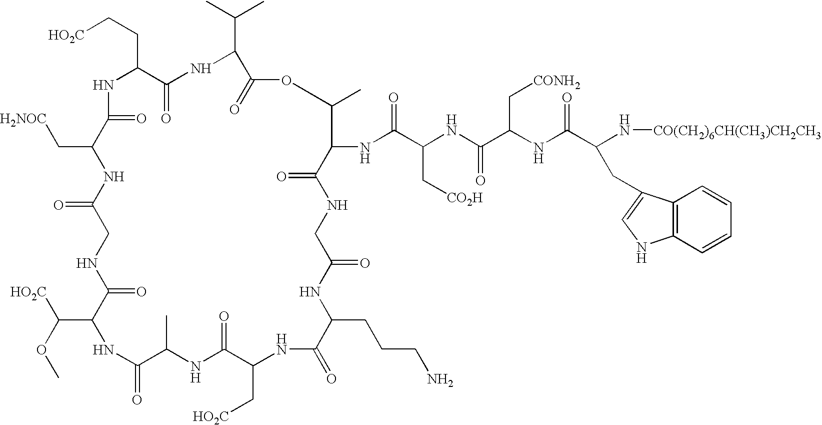 |
| C287 | 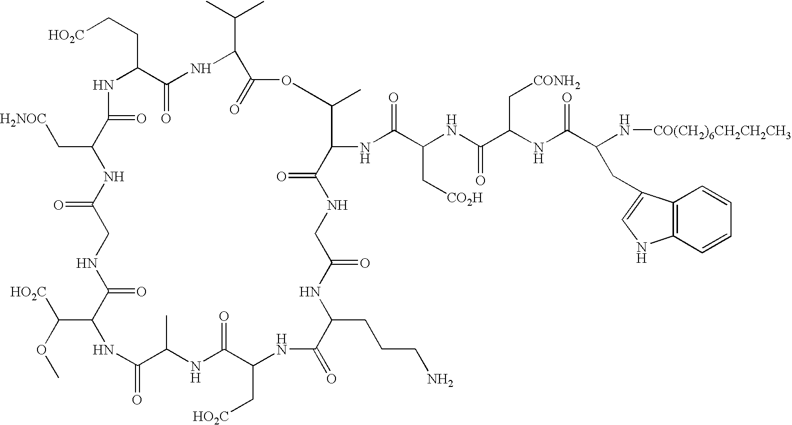 |
| C288 | 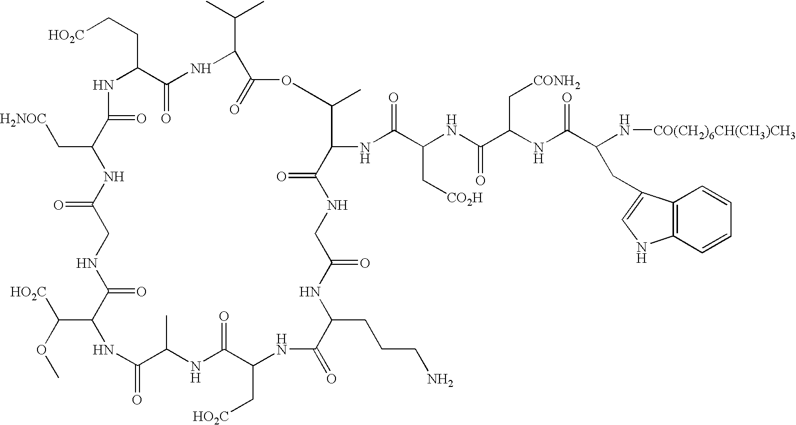 |
| C289 | 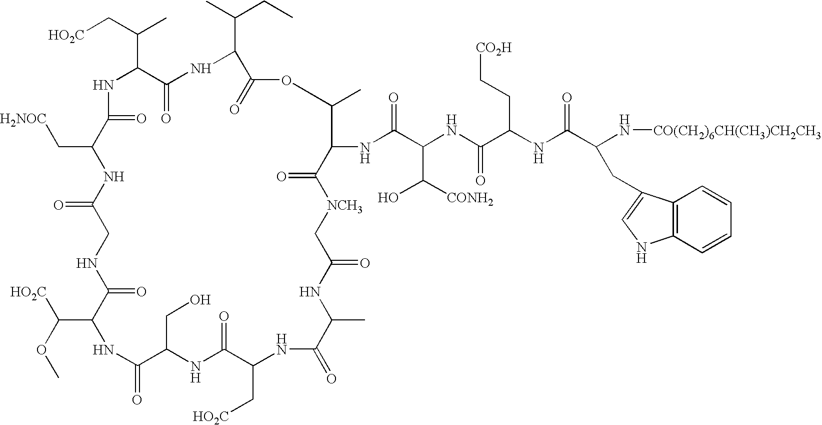 |
| C290 | 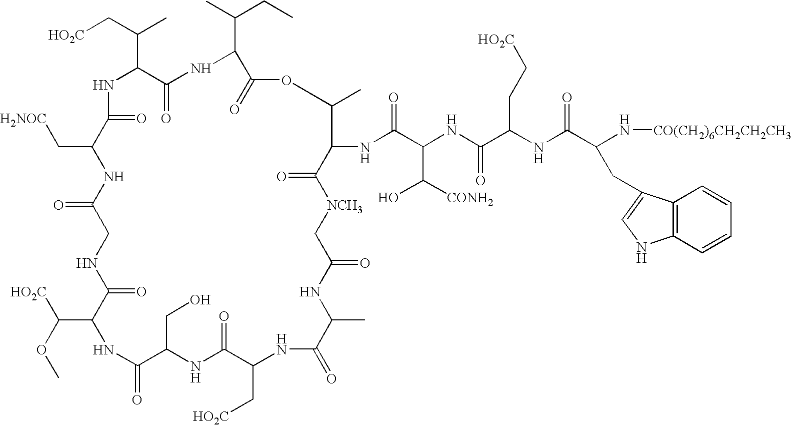 |
| C291 | 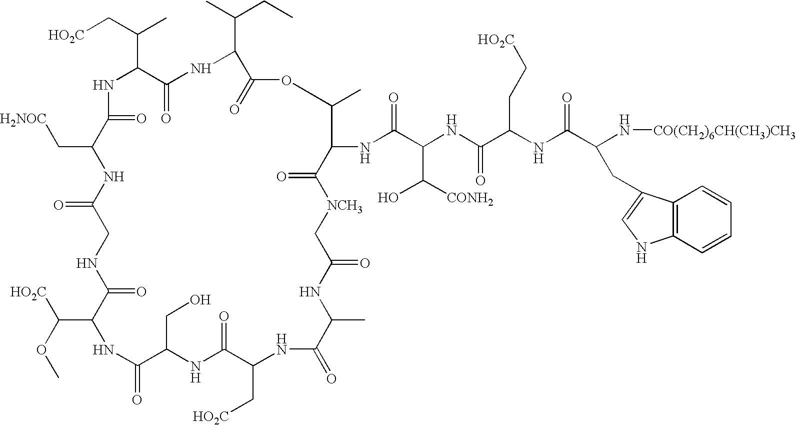 |
| C292 | 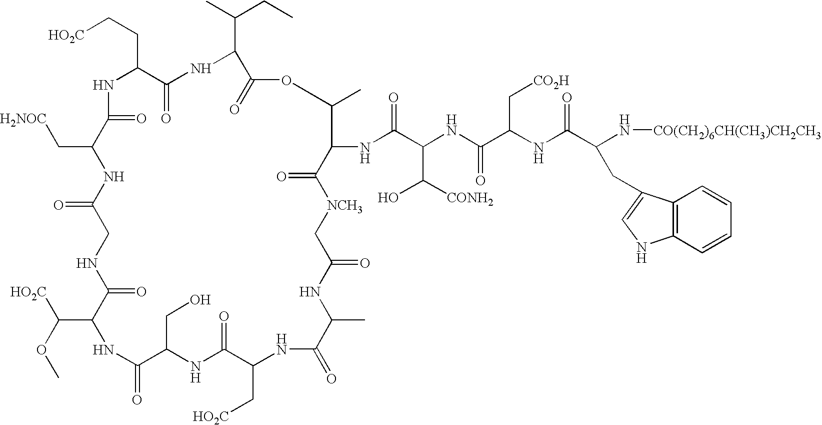 |
| C293 | 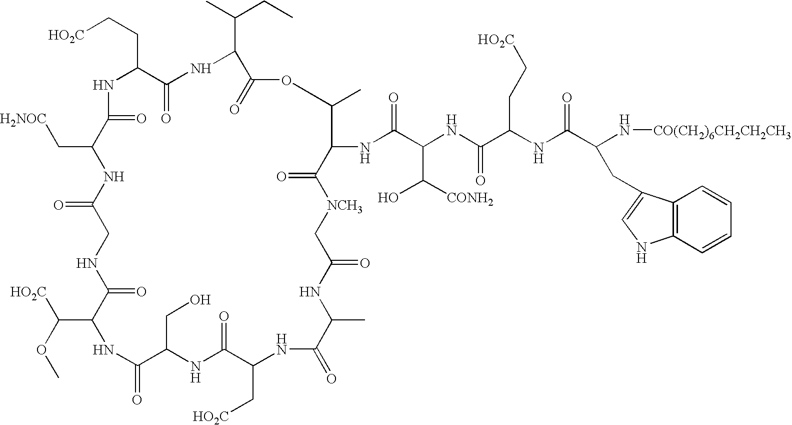 |
| C294 | 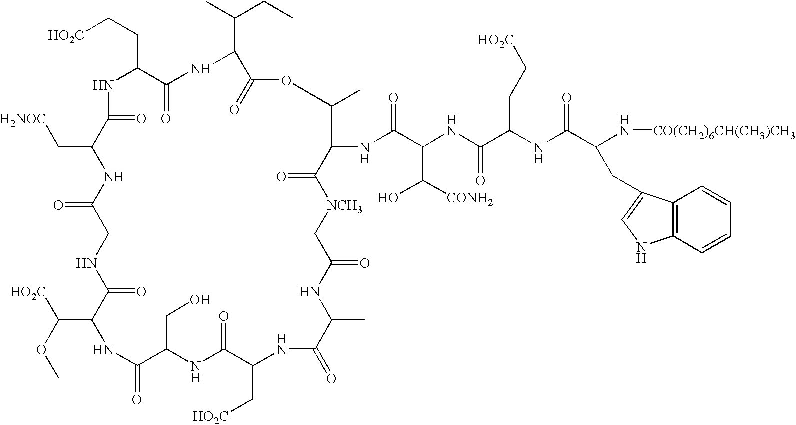 |
| C295 | 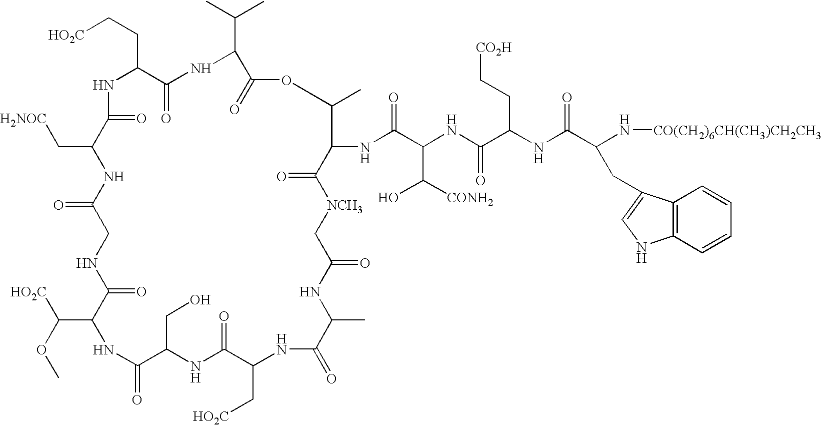 |
| C296 | 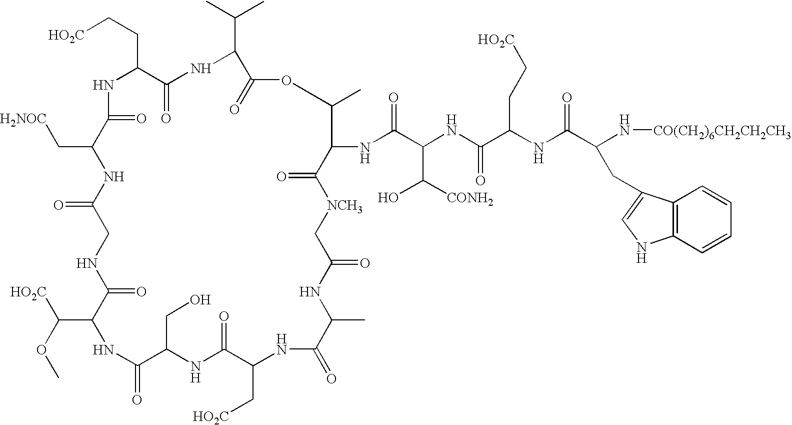 |
| C297 | 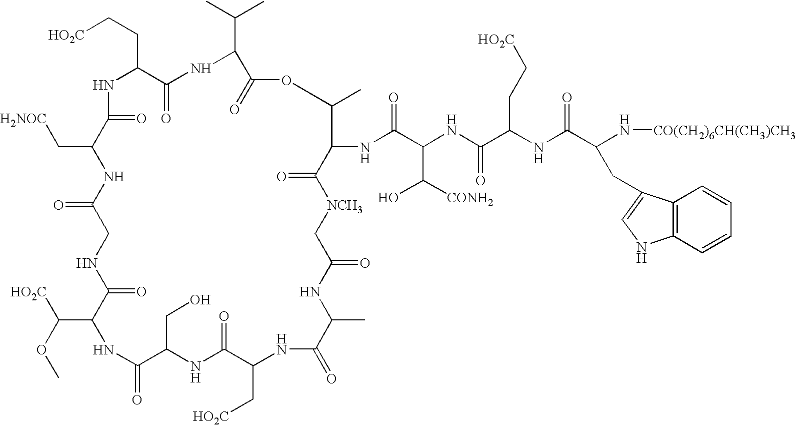 |
| C298 | 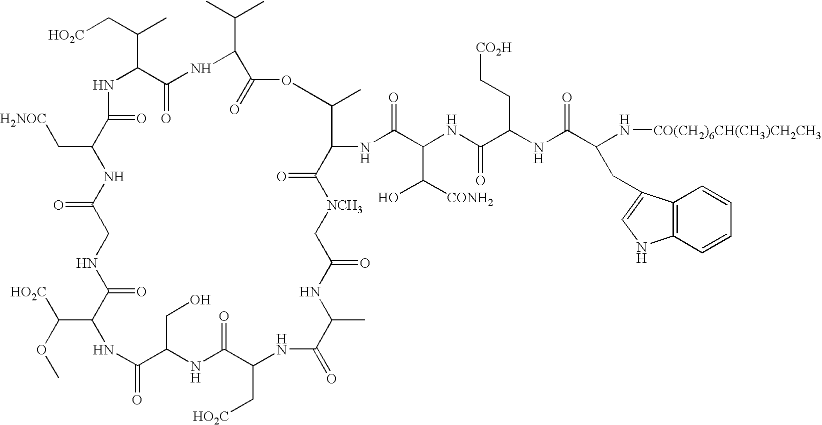 |
| C299 | 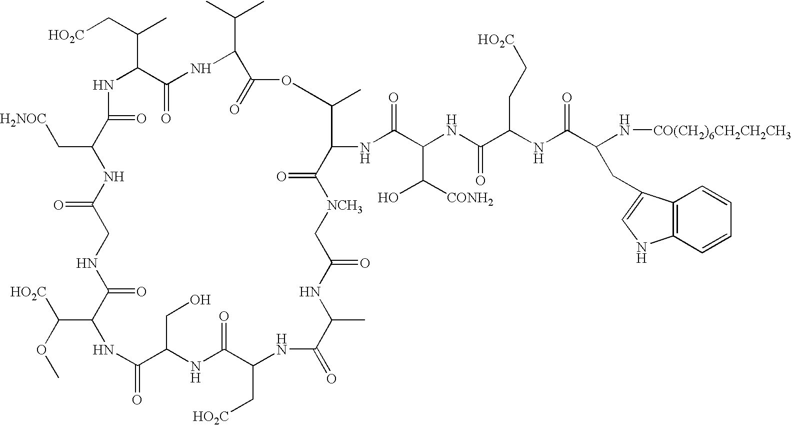 |
| C300 | 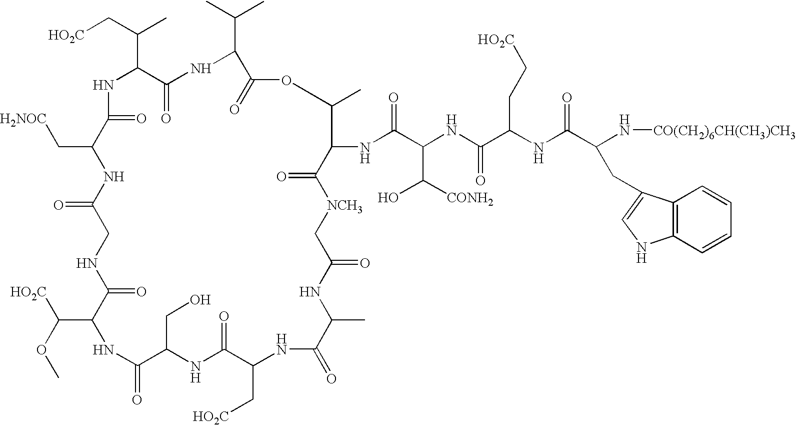 |
| C301 | 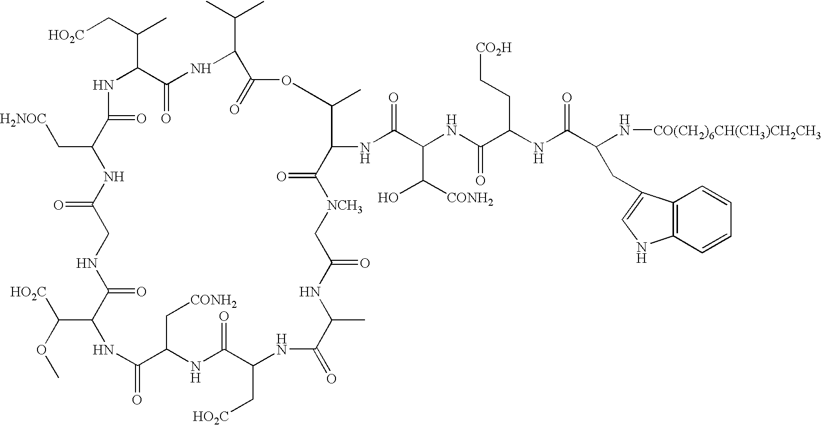 |
| C302 | 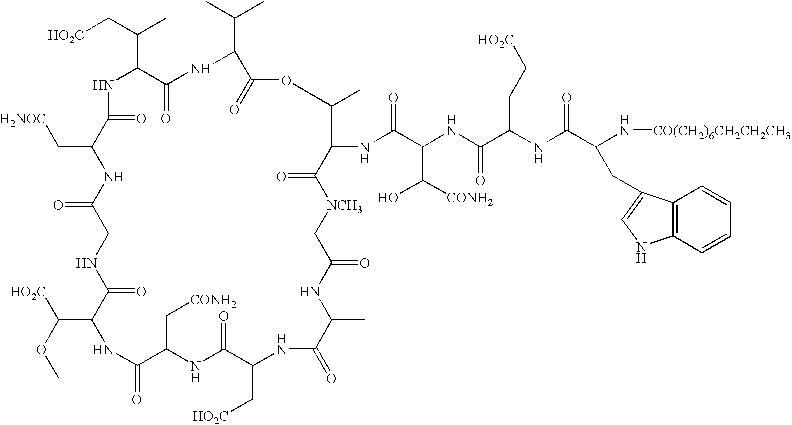 |
| C303 | 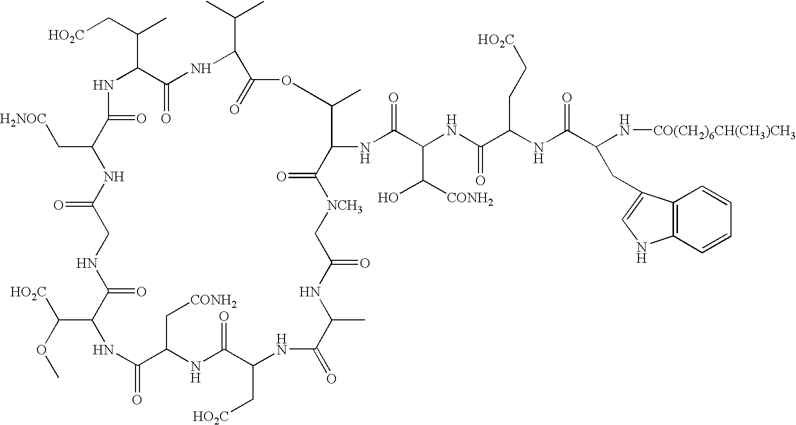 |
| C304 | 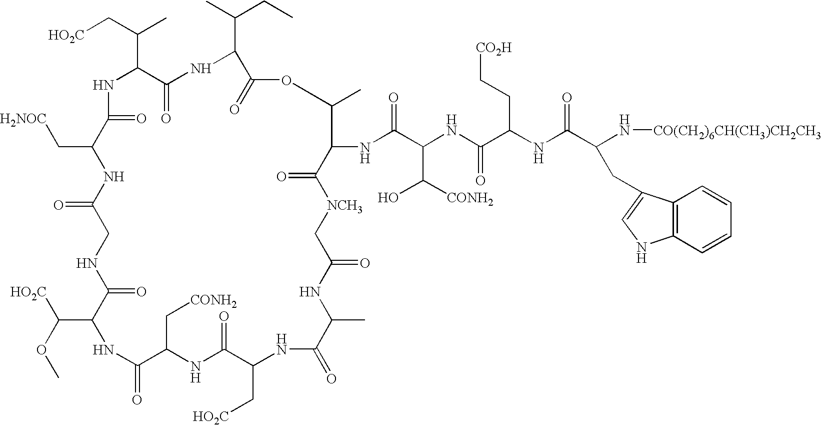 |
| C305 | 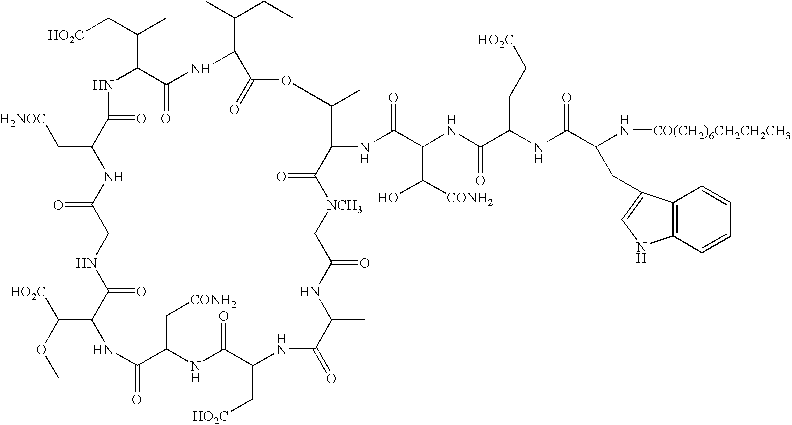 |
| C306 | 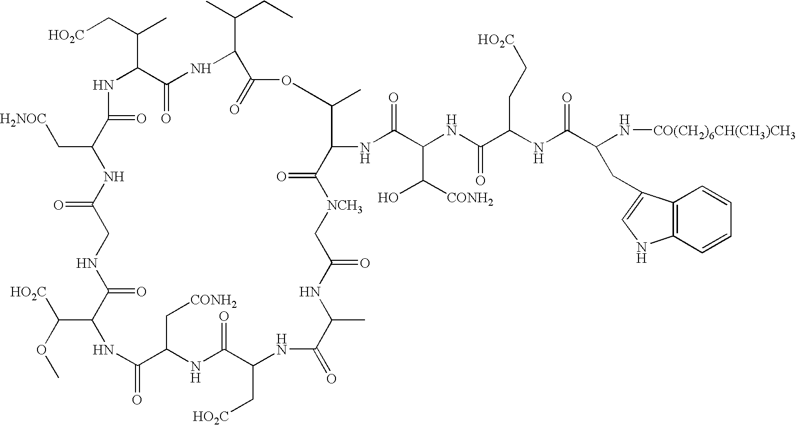 |
| C307 | 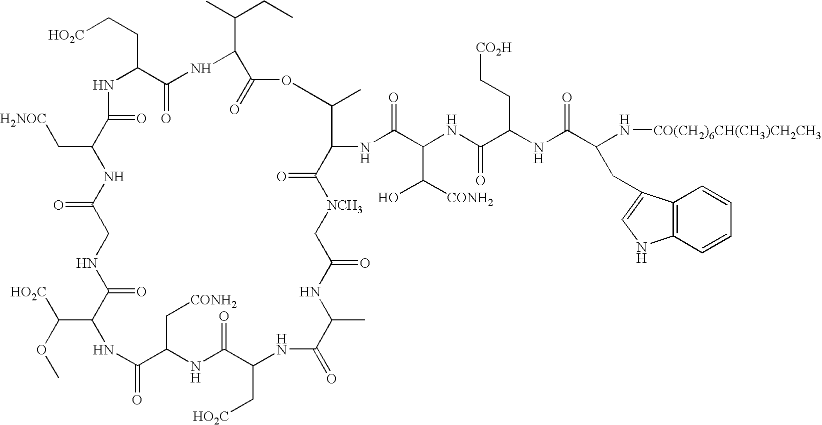 |
| C308 | 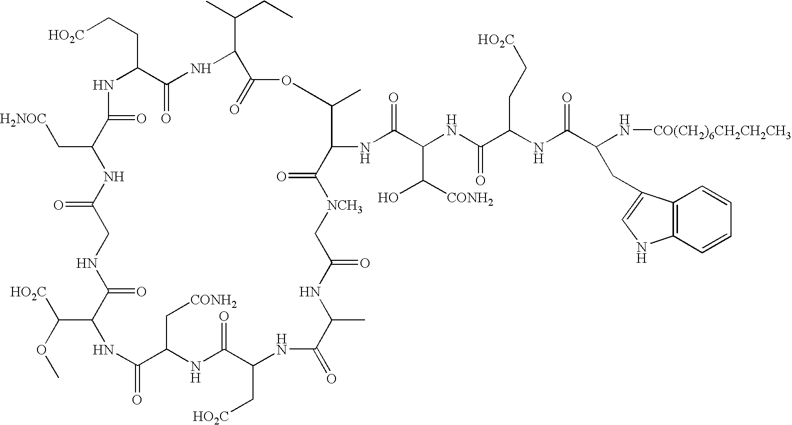 |
| C309 | 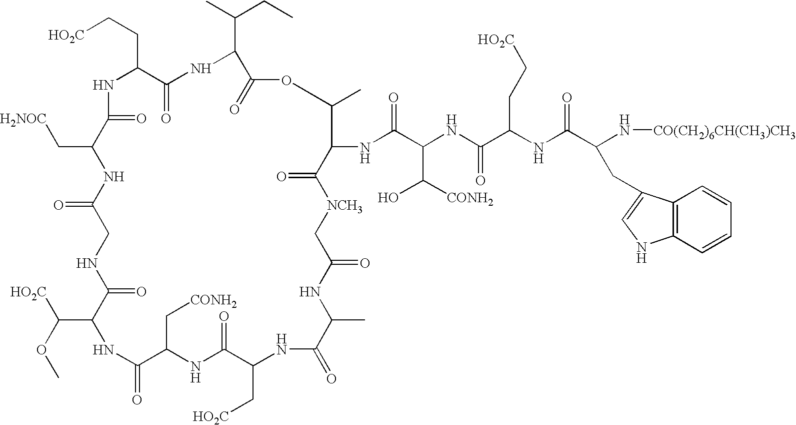 |
| C310 | 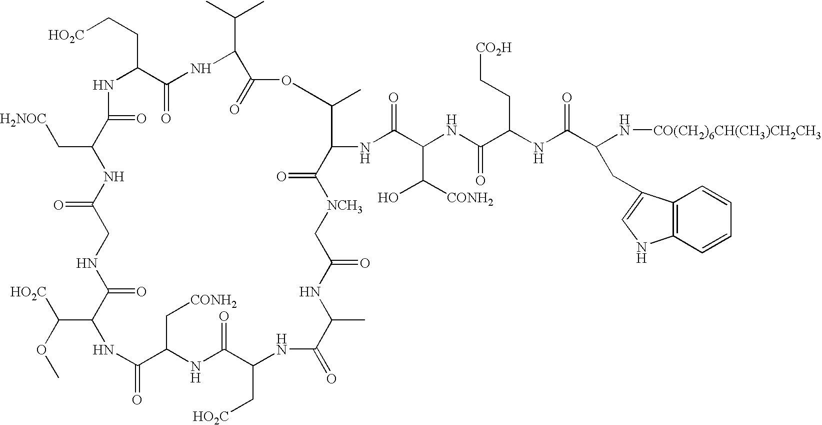 |
| C311 | 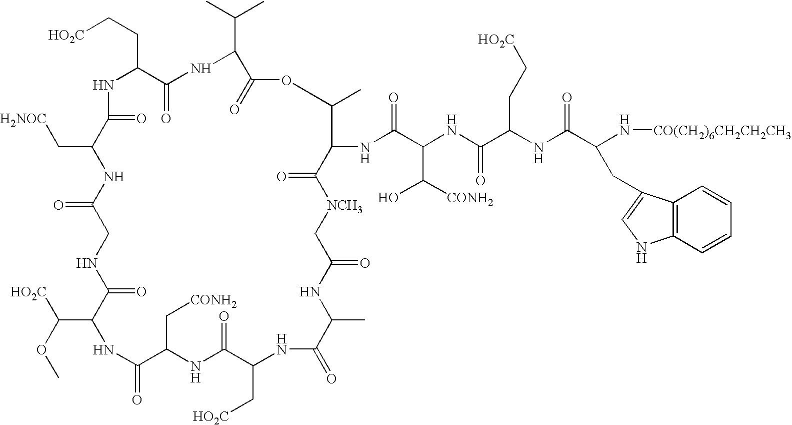 |
| C312 | 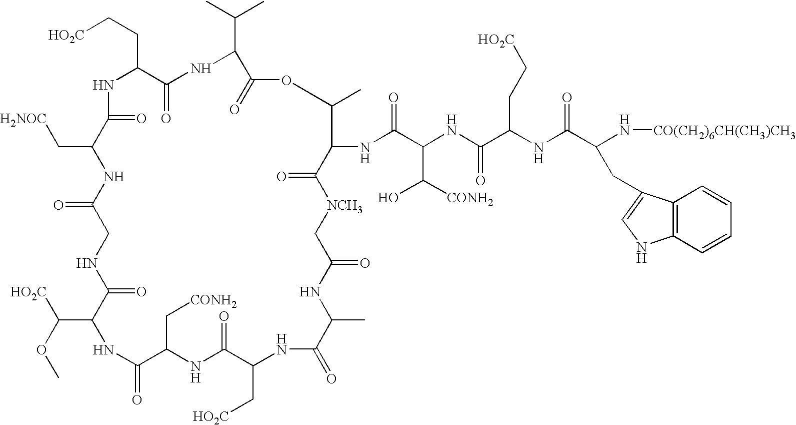 |
| C313 | 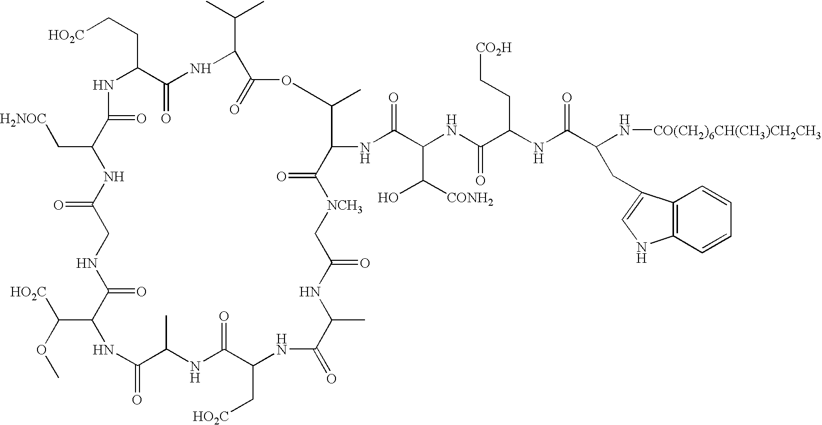 |
| C314 | 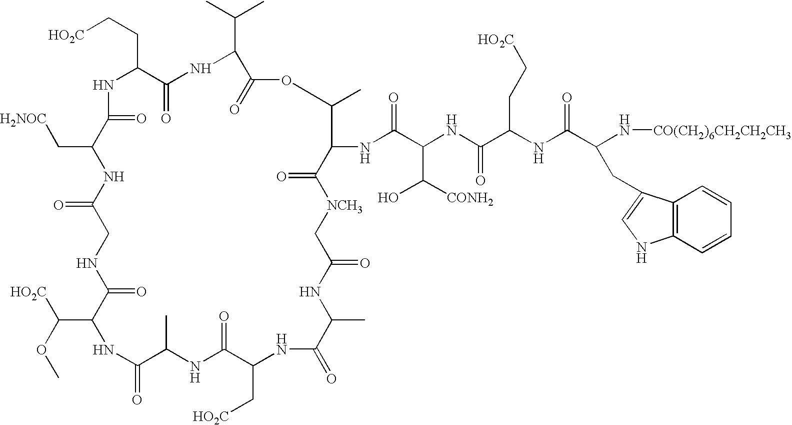 |
| C315 | 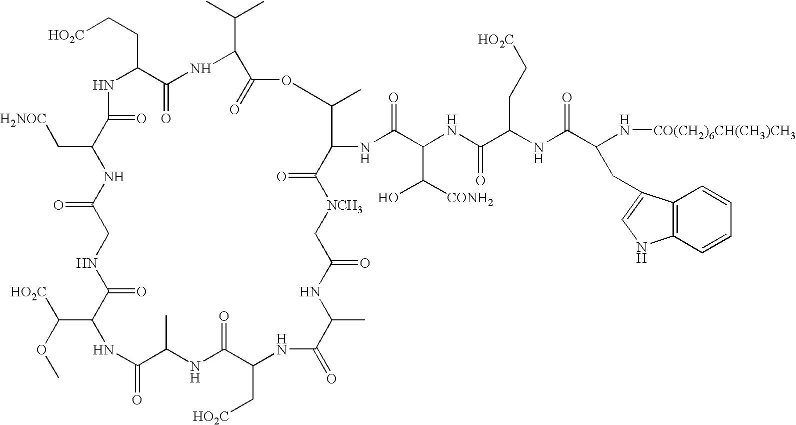 |
| C316 | 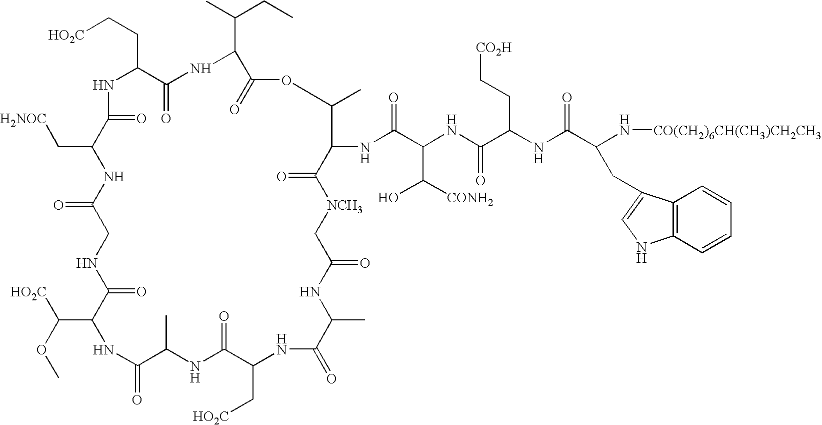 |
| C317 | 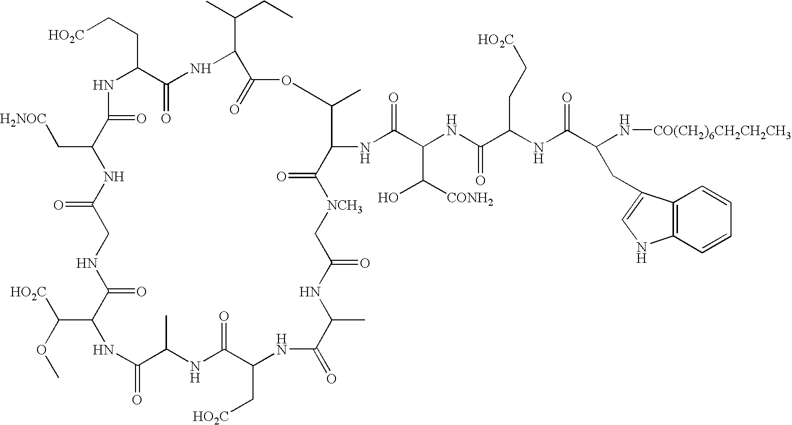 |
| C318 | 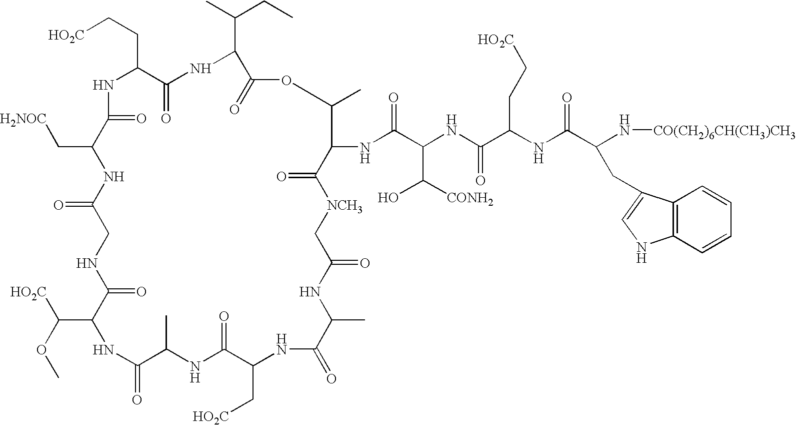 |
| C319 | 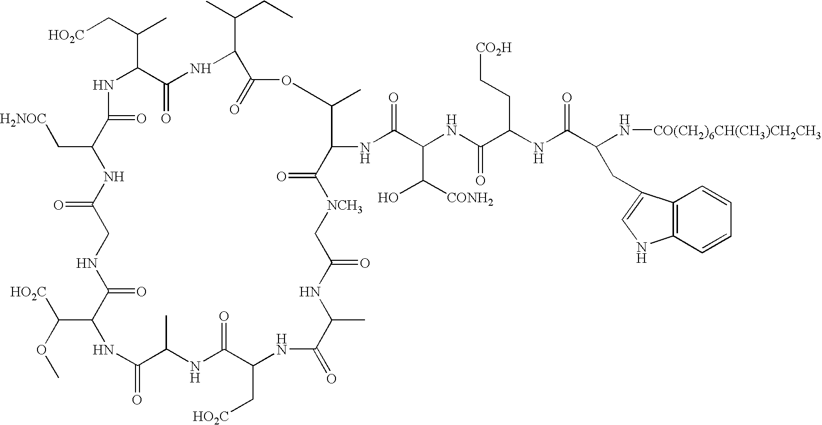 |
| C320 | 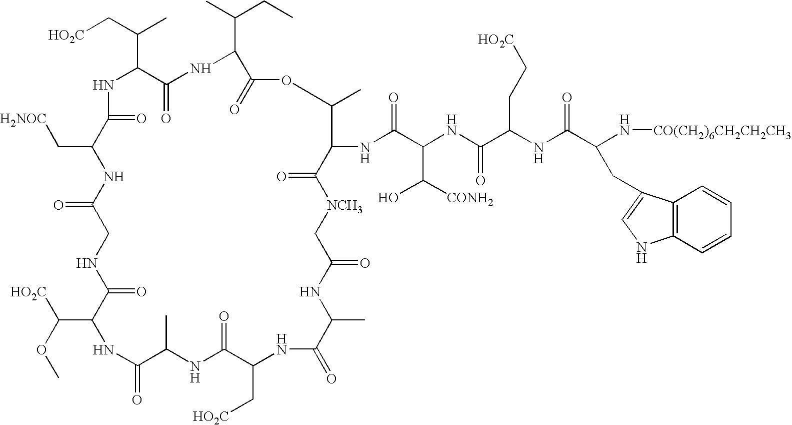 |
| C321 | 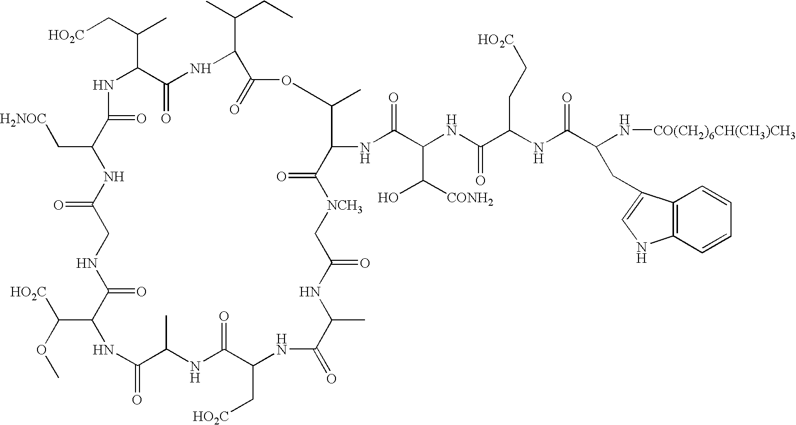 |
| C322 | 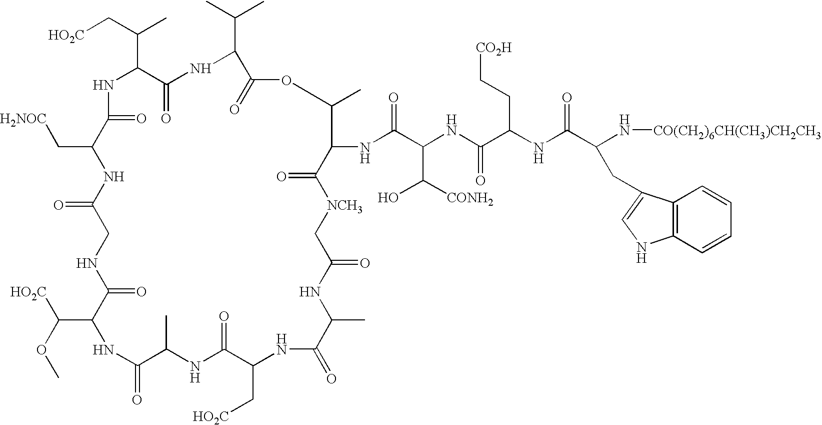 |
| C323 | 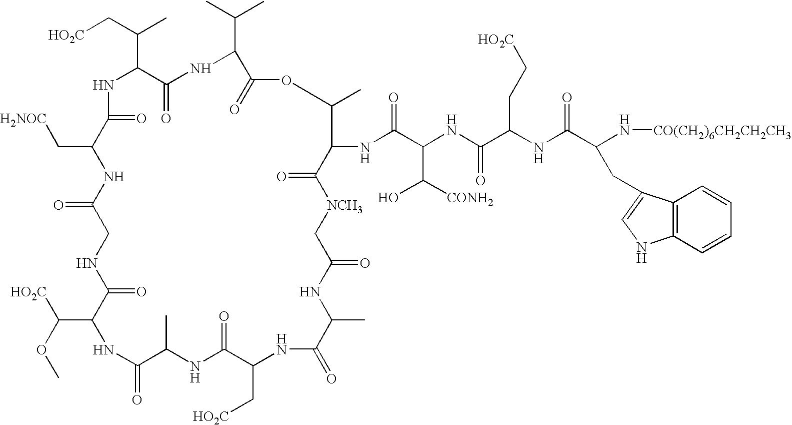 |
| C324 | 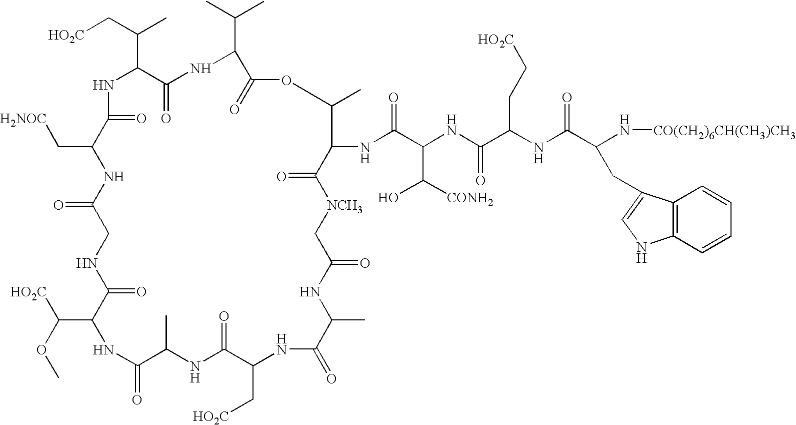 |
| C325 | 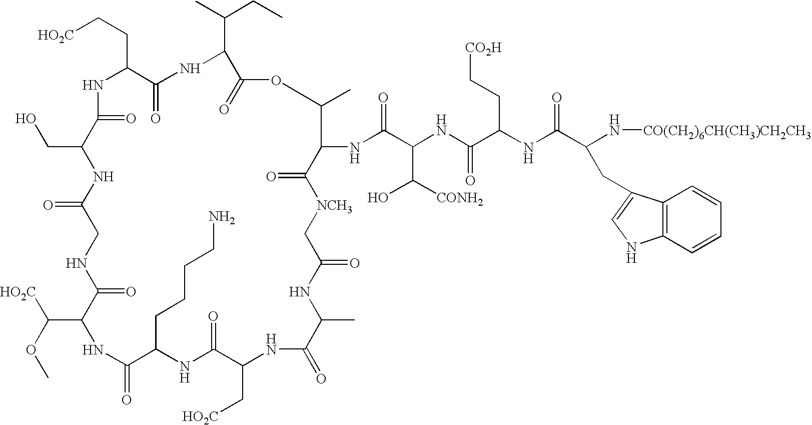 |
| C326 | 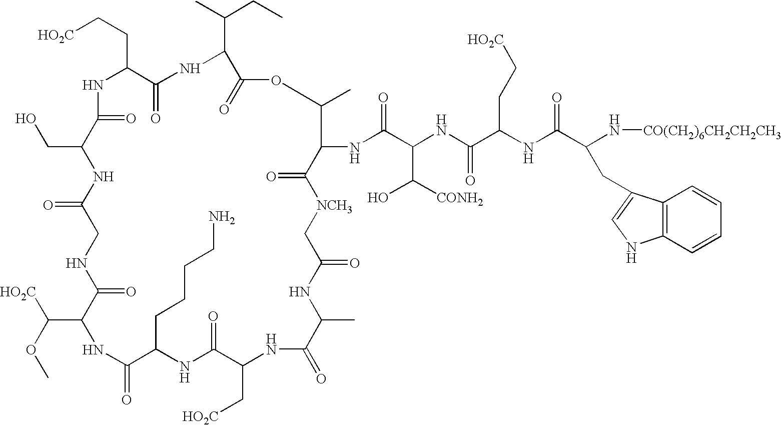 |
| C327 | 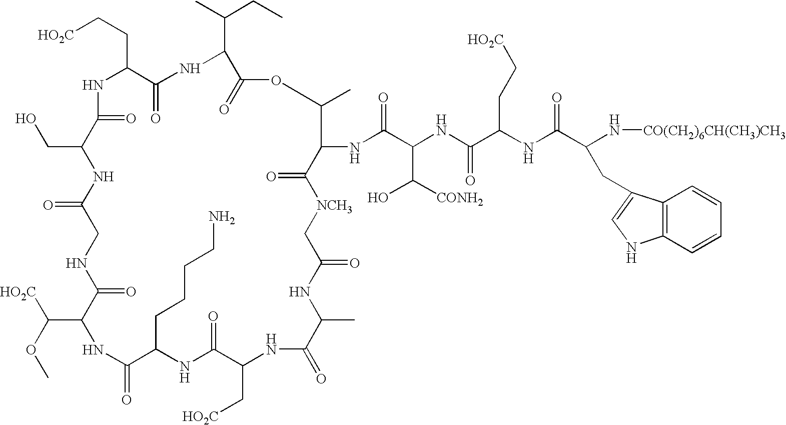 |
| C328 | 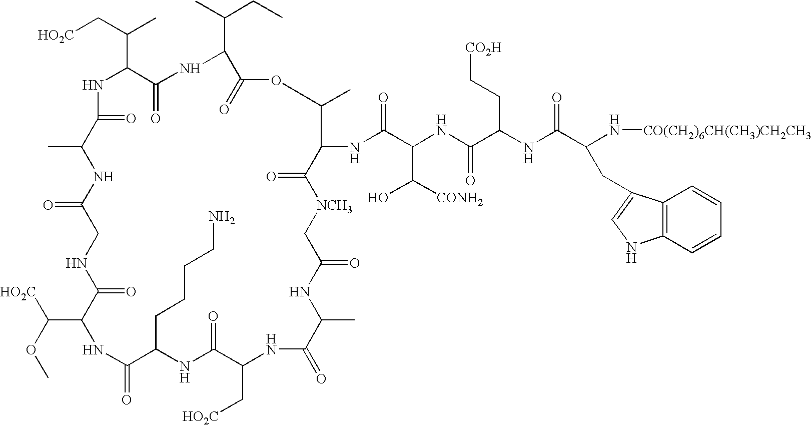 |
| C329 | 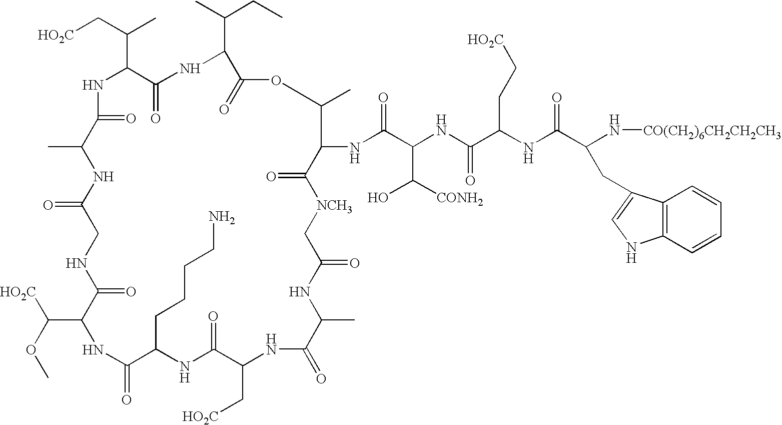 |
| C330 | 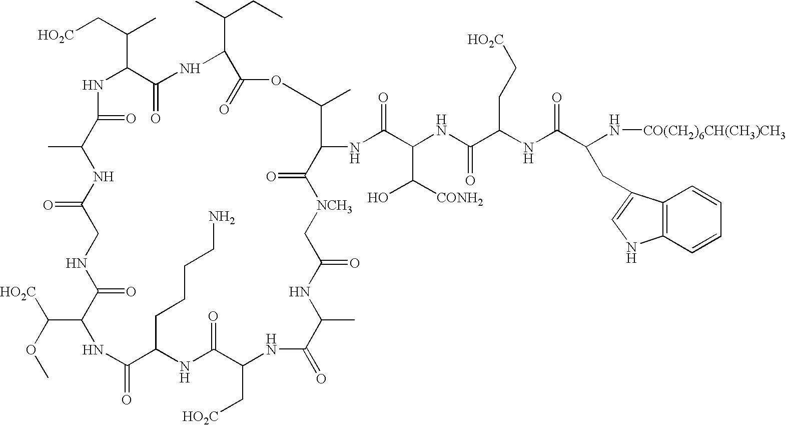 |
| C331 | 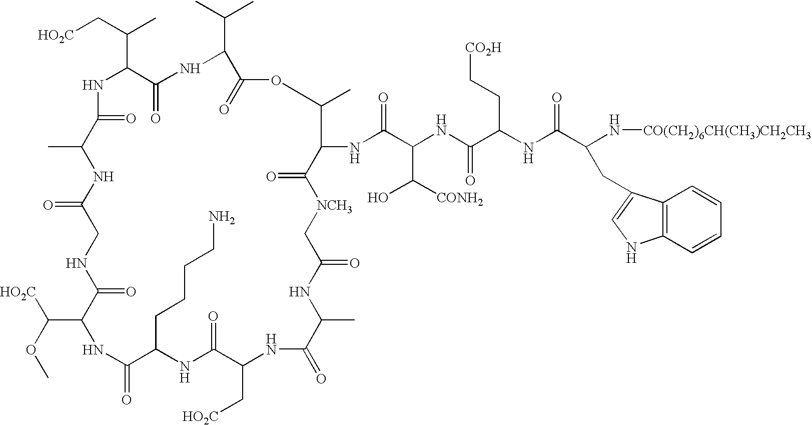 |
| C332 | 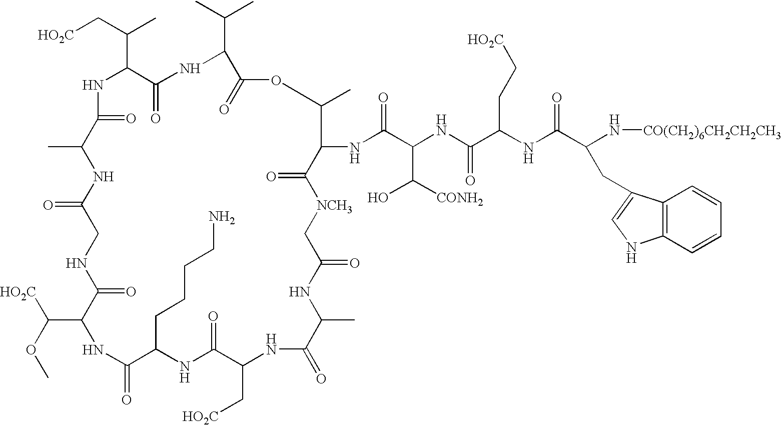 |
| C333 | 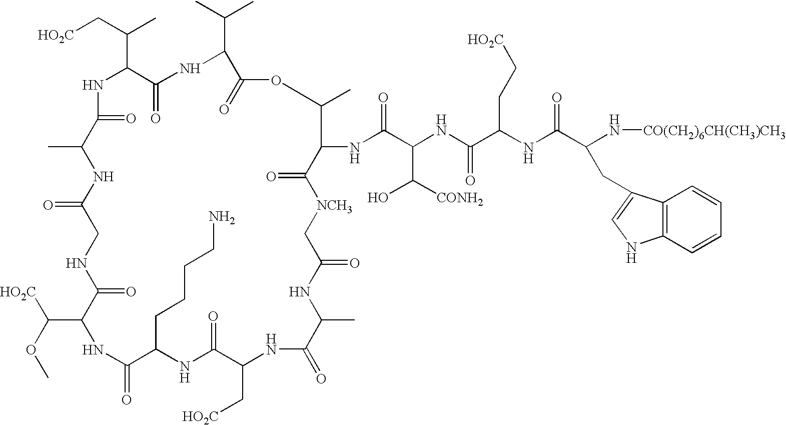 |
| C334 | 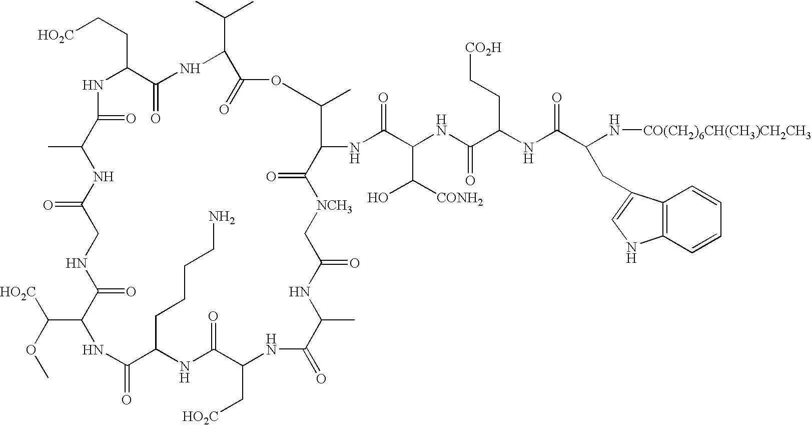 |
| C335 | 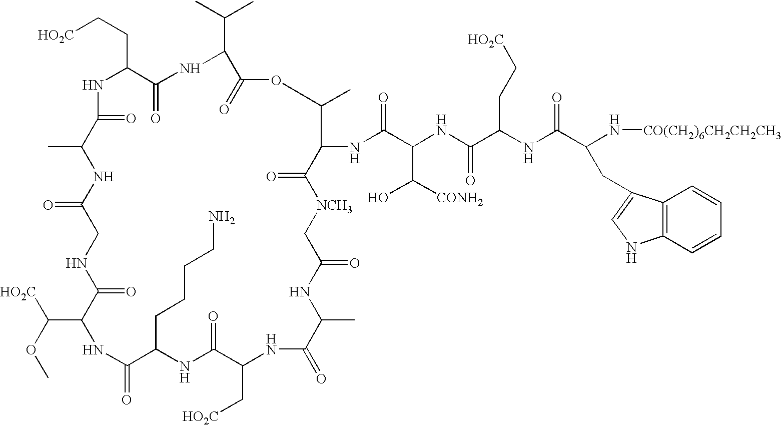 |
| C336 | 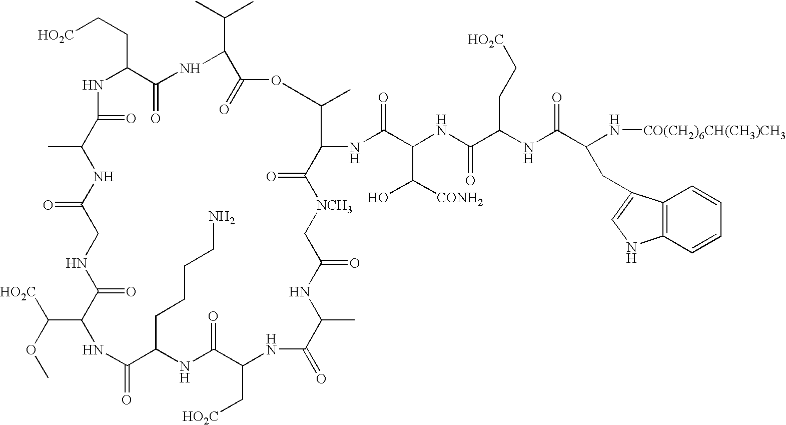 |
| C337 | 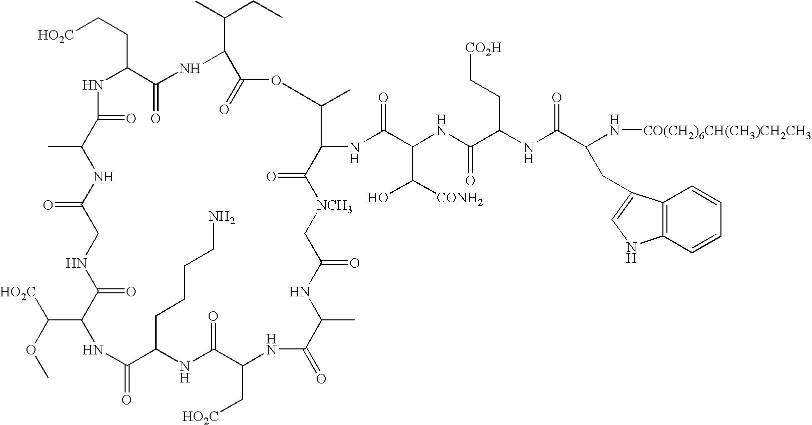 |
| C338 | 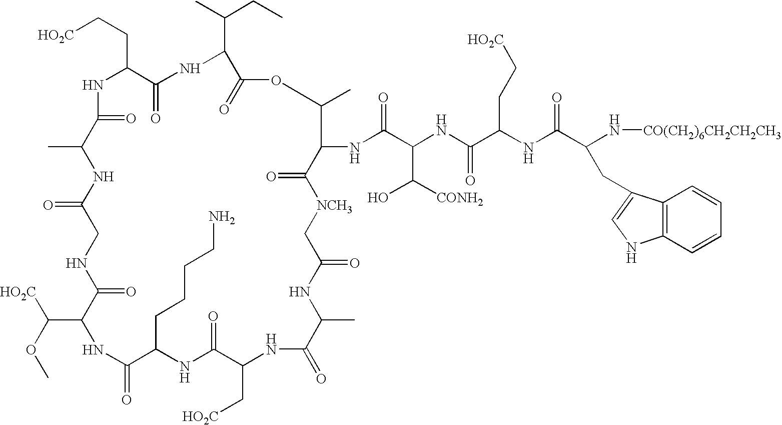 |
| C339 | 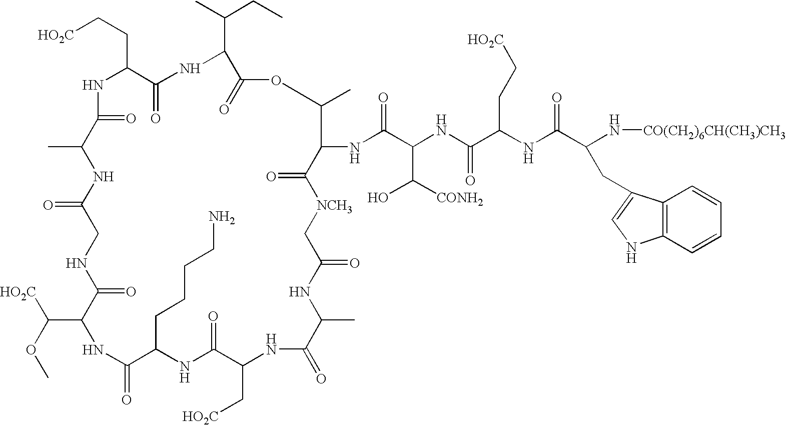 |
| C340 | 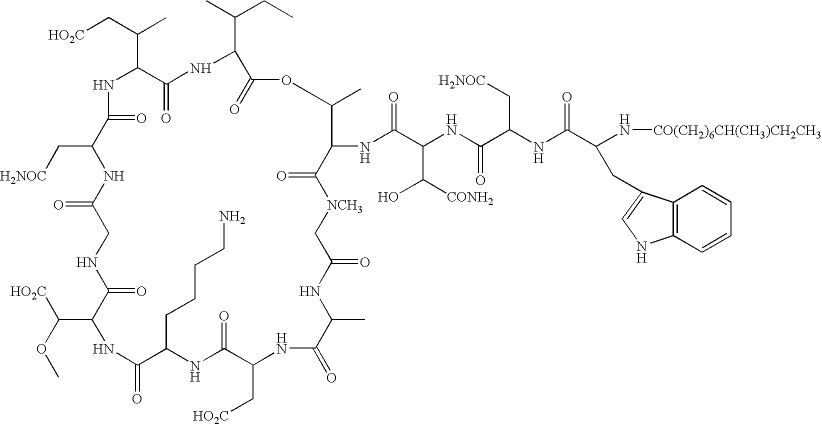 |
| C341 | 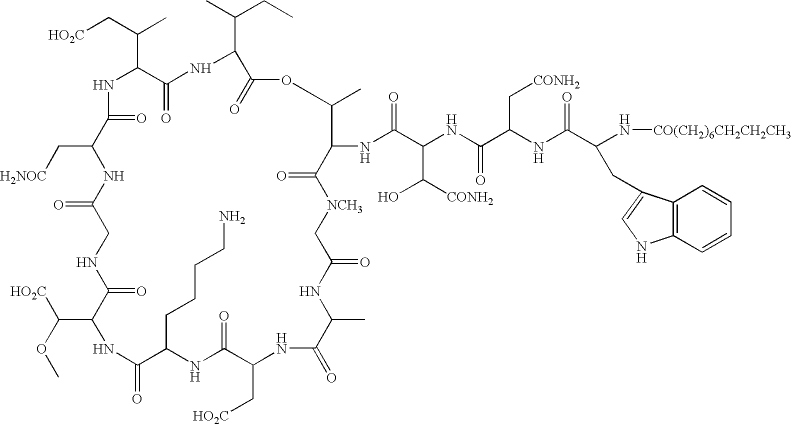 |
| C342 | 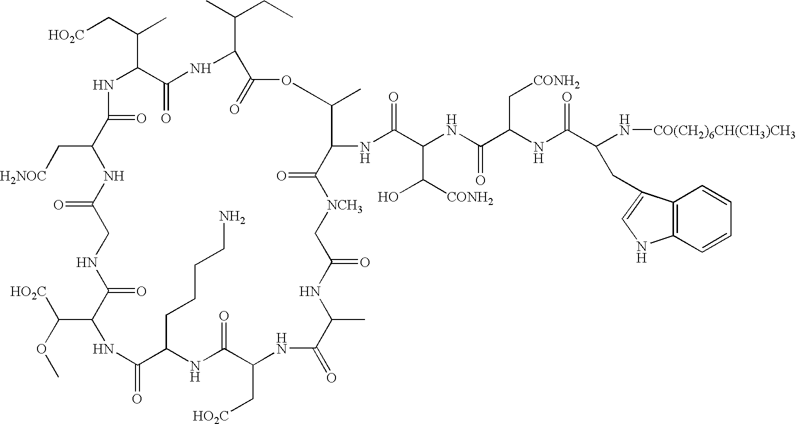 |
| C343 | 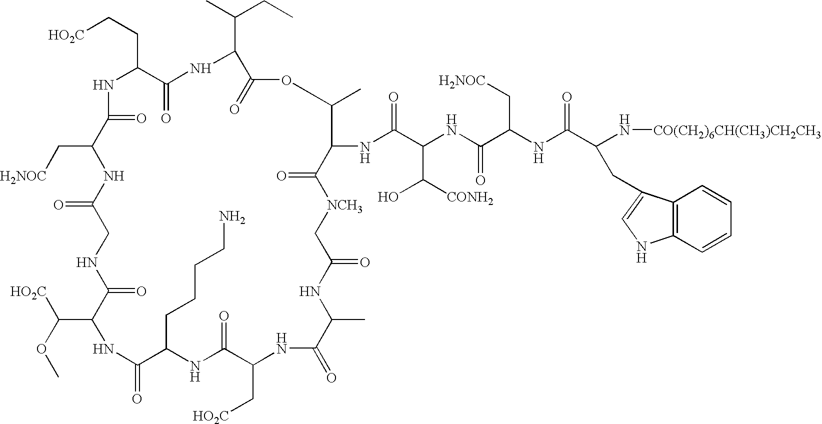 |
| C344 | 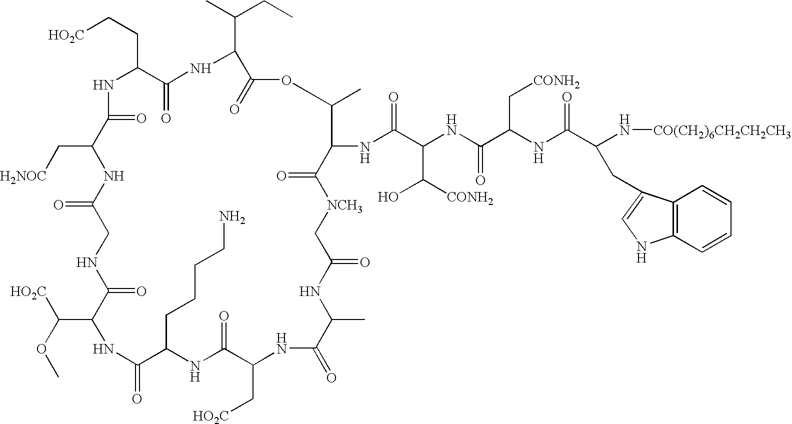 |
| C345 | 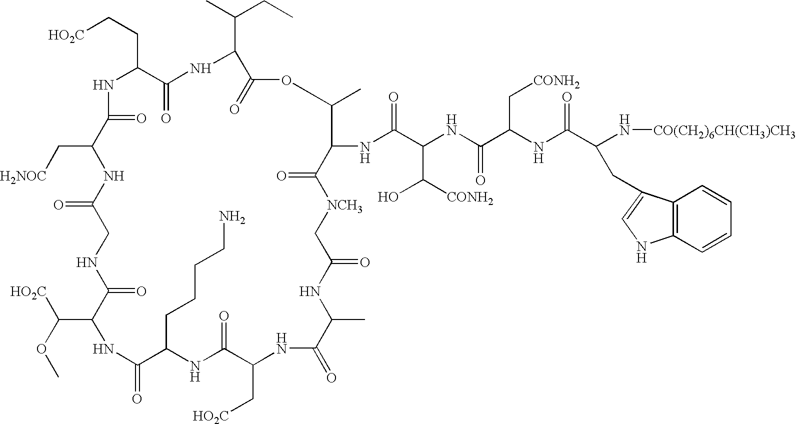 |
| C346 | 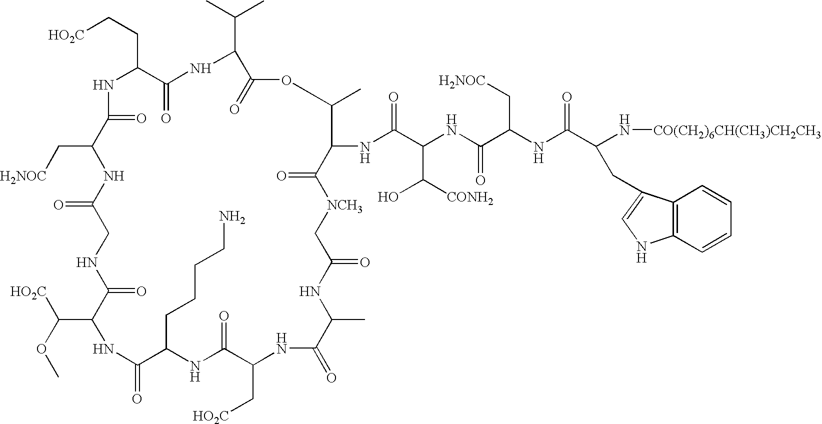 |
| C347 | 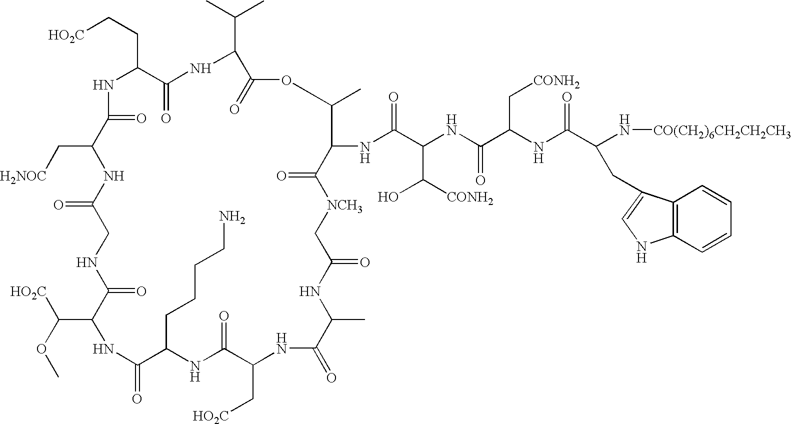 |
| C348 | 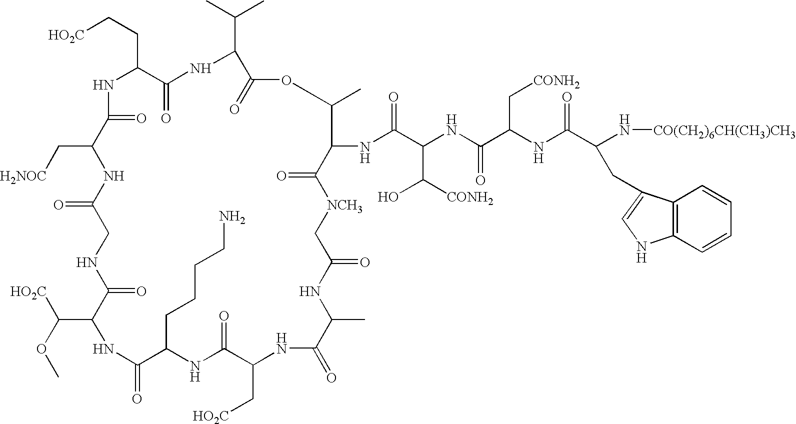 |
| C349 | 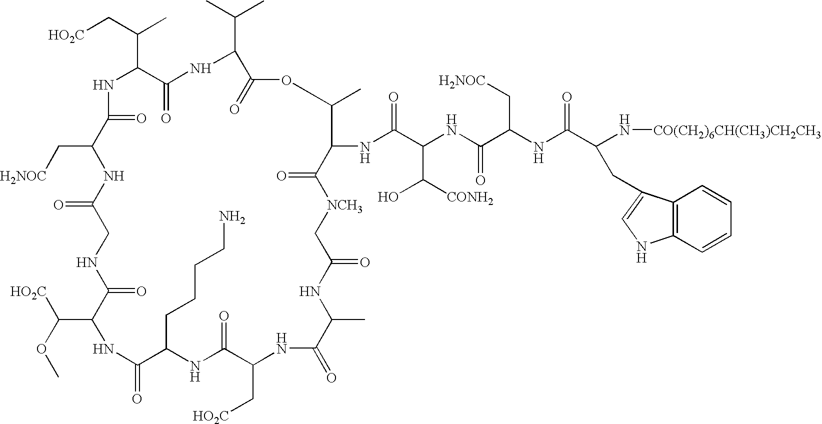 |
| C350 | 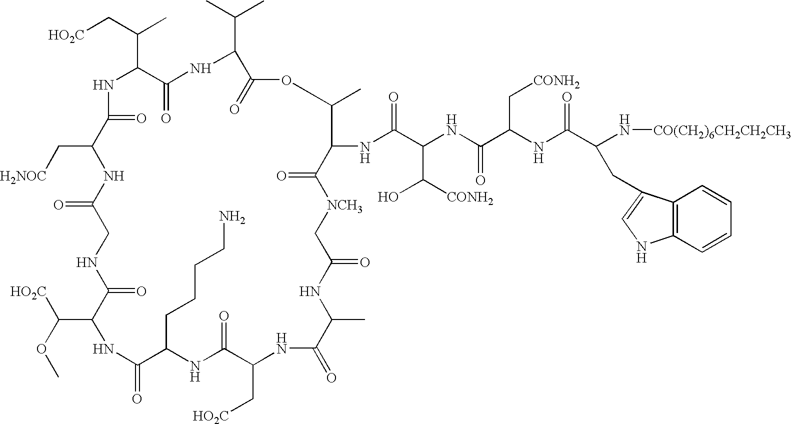 |
| C351 | 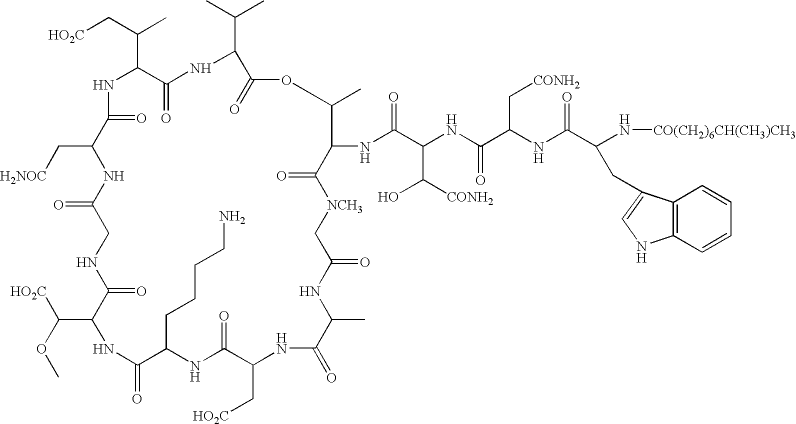 |
| C352 | 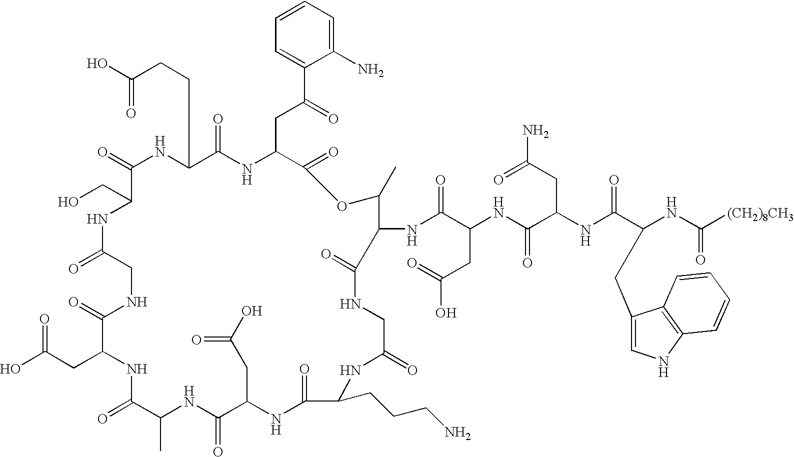 |
| C353 | 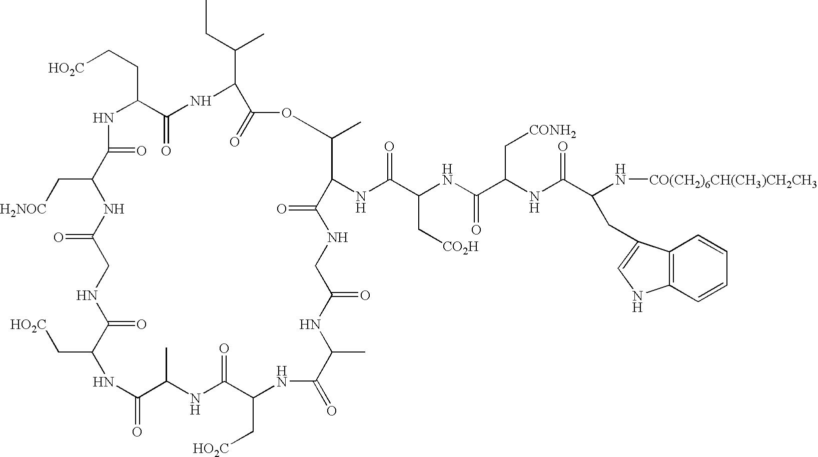 |
| C354 | 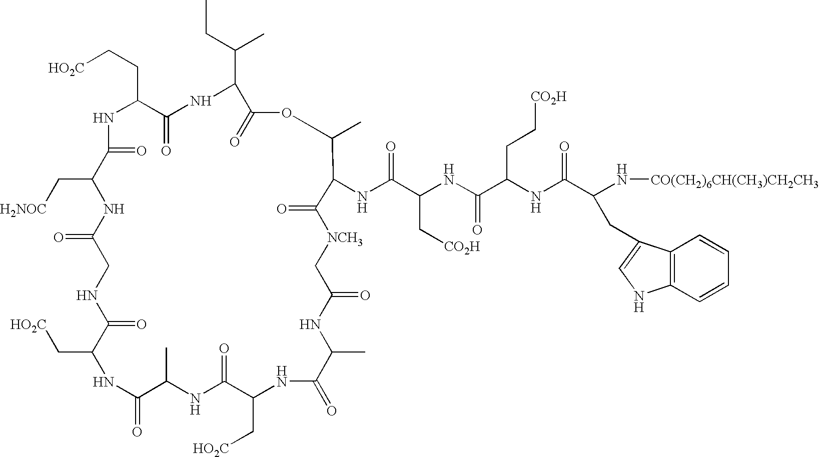 |
| C355 | 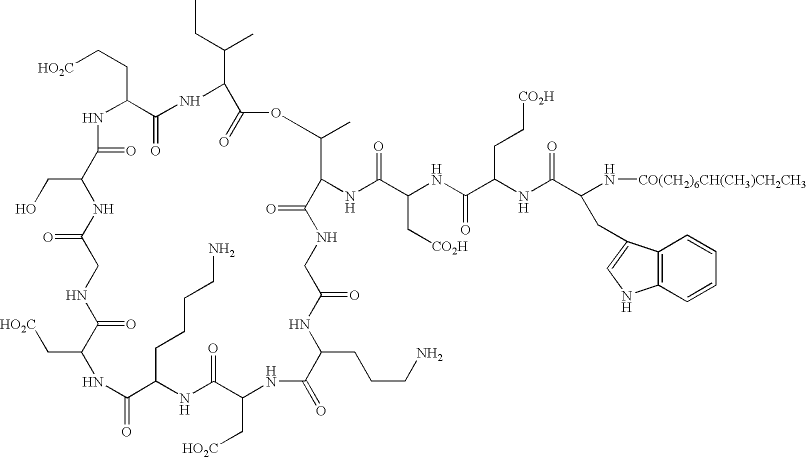 |
| C356 | 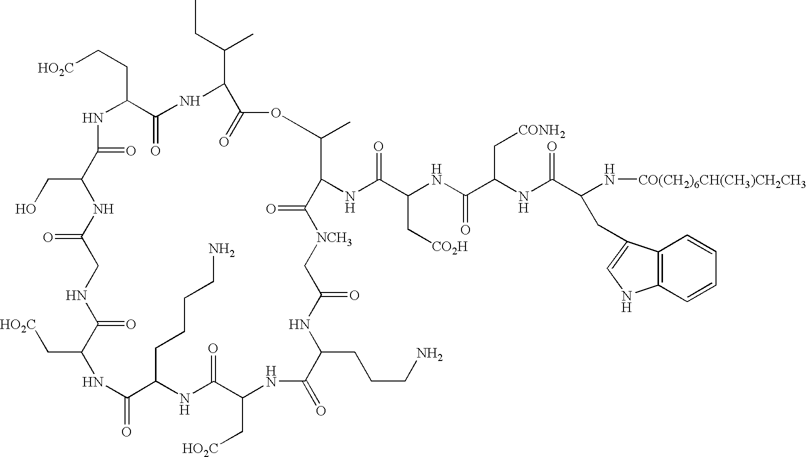 |
| C357 | 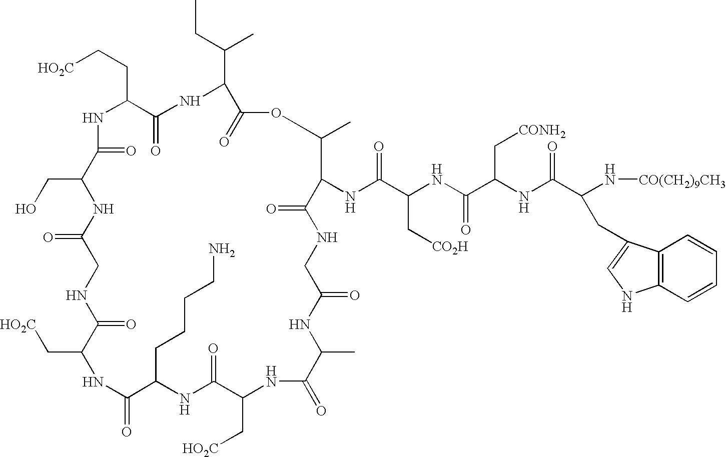 |
| C358 | 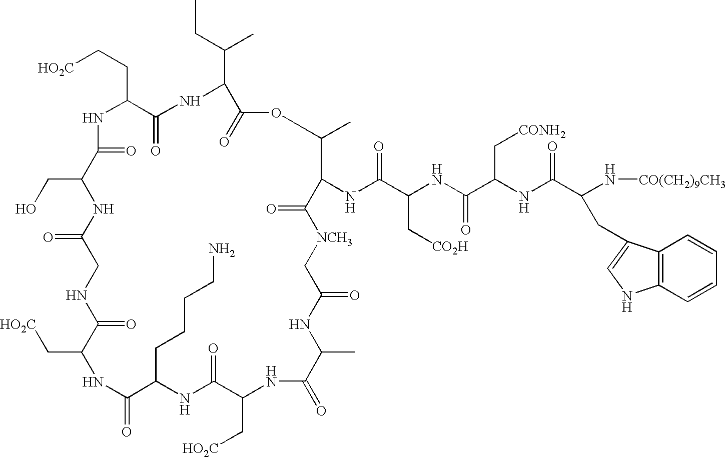 |
| C359 | 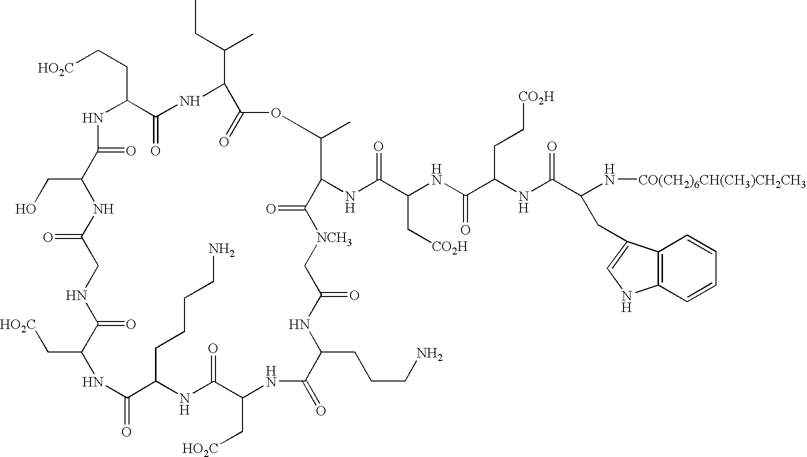 |
| C360 | 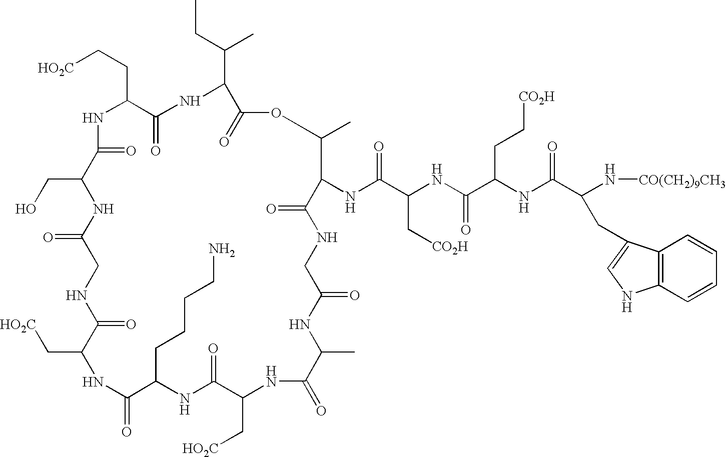 |
| C361 | 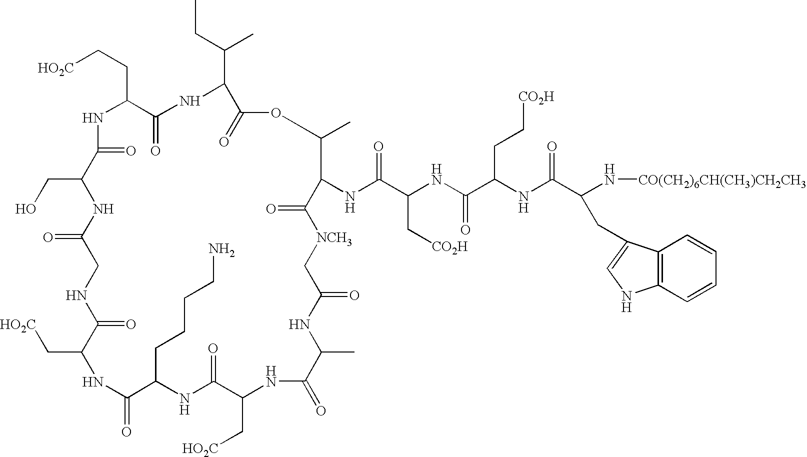 |
| C362 | 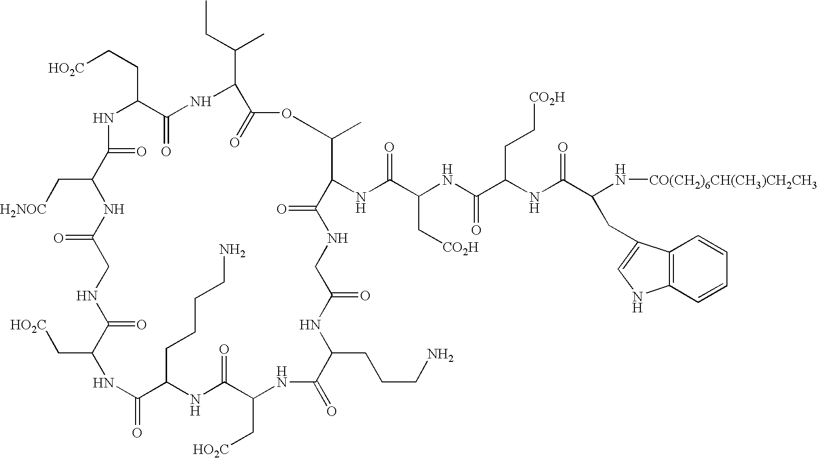 |
| C363 | 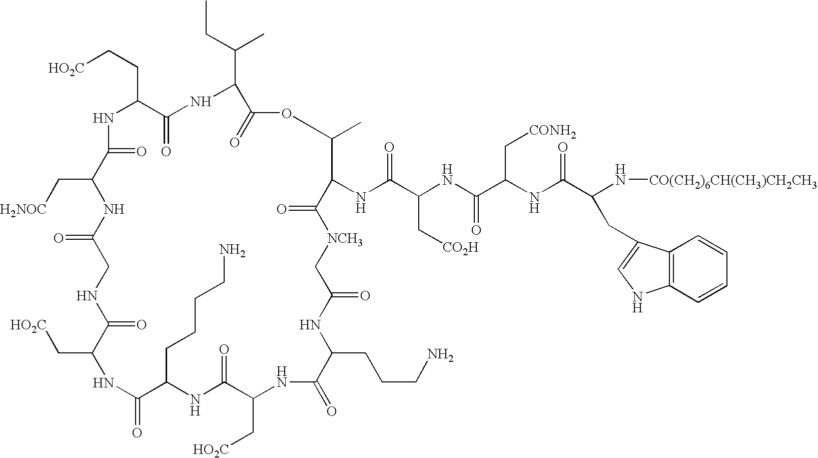 |
| C364 | 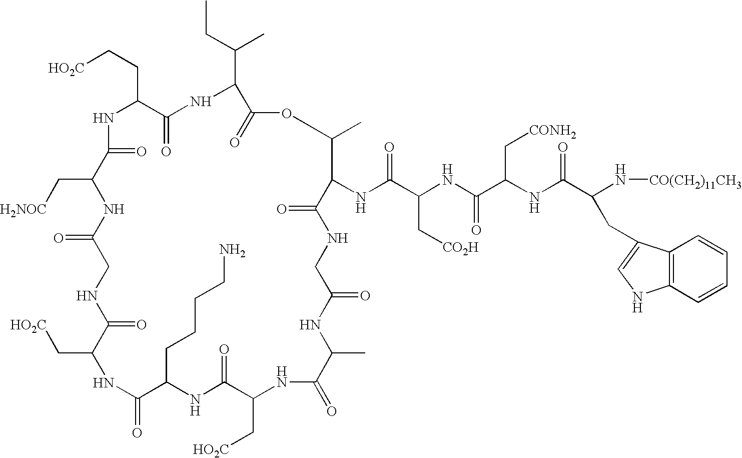 |
| C365 | 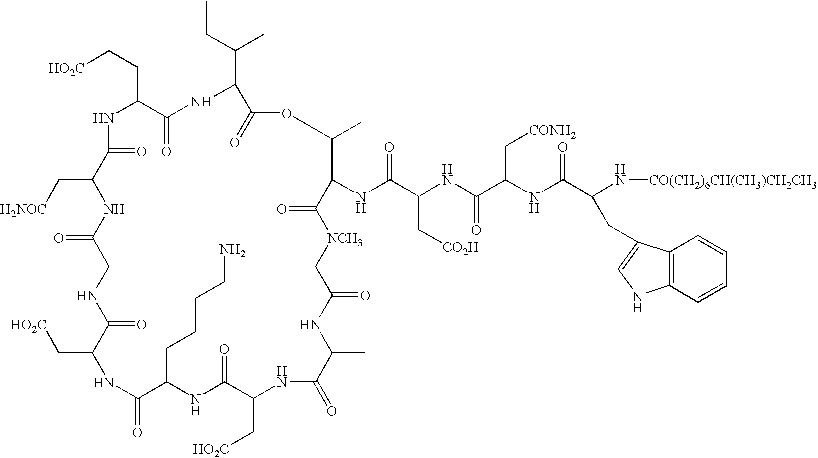 |
| C366 | 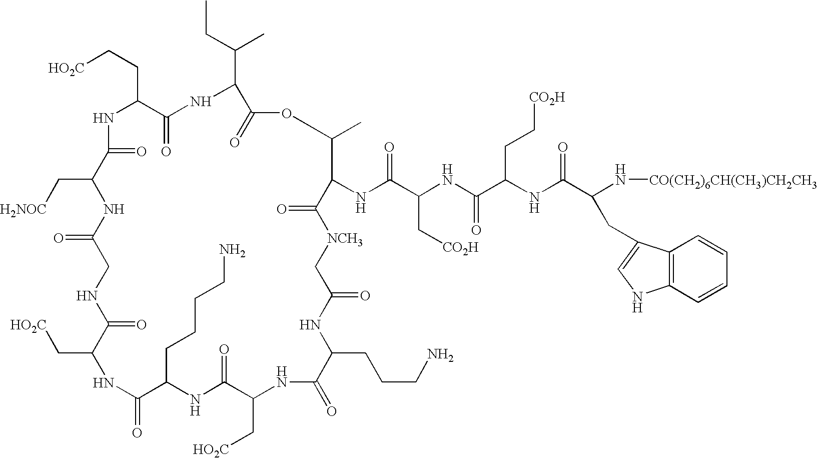 |
| C367 | 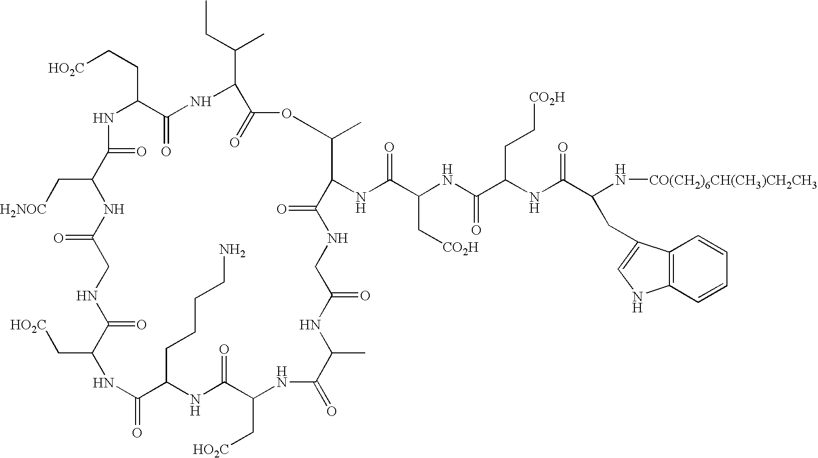 |
| C368 | 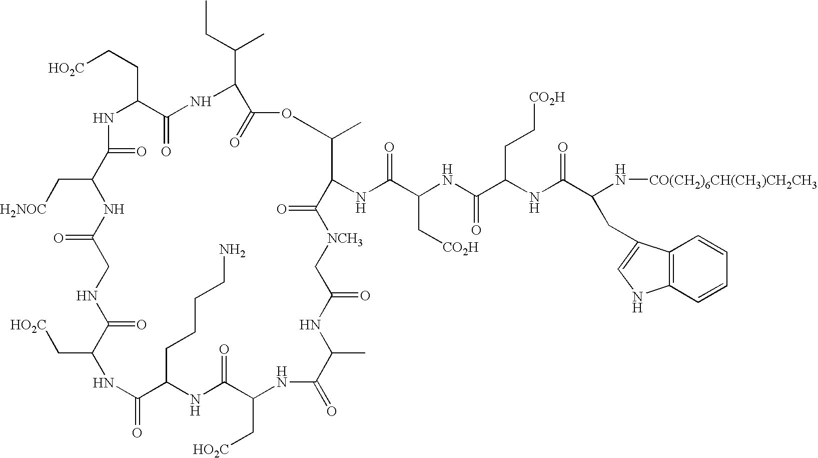 |
| C369 | 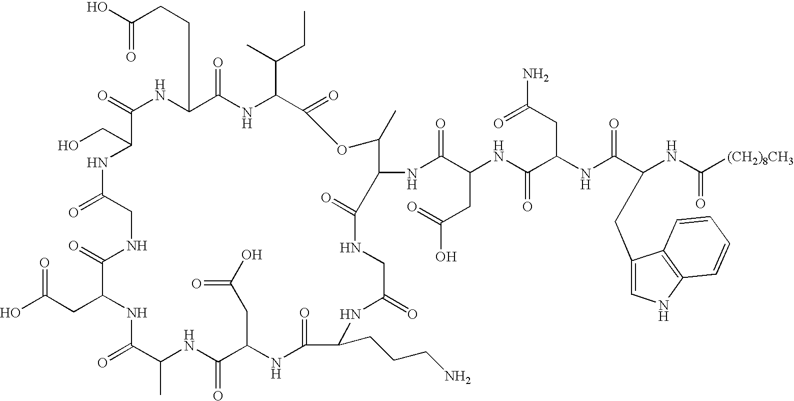 |

In one embodiment of the invention, each of R²*, R⁵*, R⁸*, R¹¹*, and R¹² is H R⁹ is 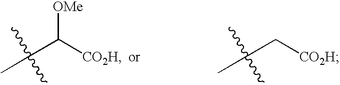
and R¹³ is CH(CH₂CH₃)CH₃. This embodiment provides a compound of Formula II. 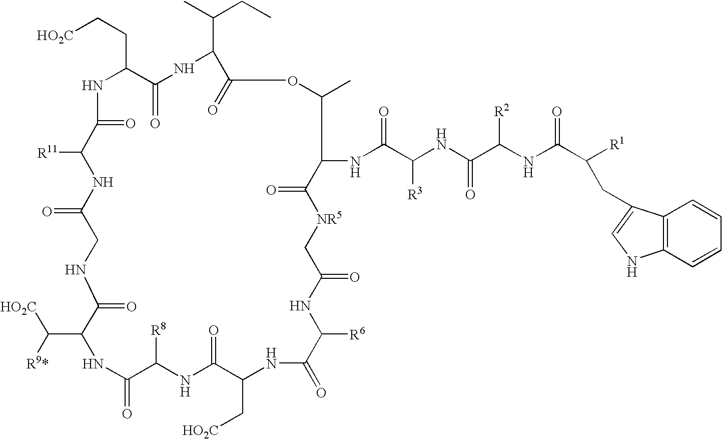
wherein R⁹* is H or OMe and R¹, R², R³, R⁵, R⁶, R⁸, and R¹¹ are as previously defined.

Table II provides exemplary compounds of Formula II.

TABLE II
Compounds of Formula II

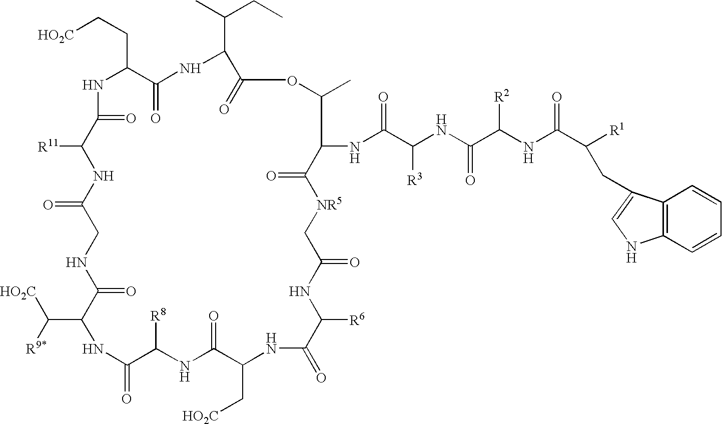

| # | R² | R³ | R⁵ | R⁶ | R⁸ | R^{9*} | R¹¹ |
|---|---|---|---|---|---|---|---|
| TII1 | 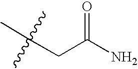 | 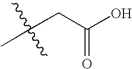 | H | 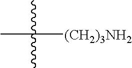 | CH₃ | H | 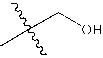 |
| TII2 | 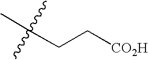 | 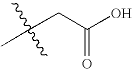 | H | 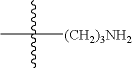 | CH₃ | H | 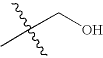 |
| TII3 | 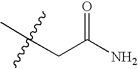 | 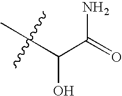 | H | 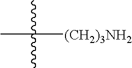 | CH₃ | H | 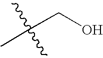 |
| TII4 | 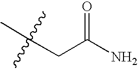 | 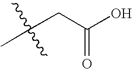 | CH₃ | 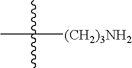 | CH₃ | H | 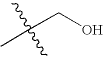 |
| TII5 | 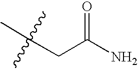 | 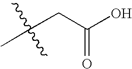 | H | CH₃ | CH₃ | H | 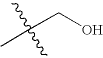 |
| TII6 | 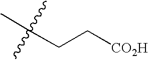 | 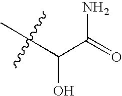 | H | 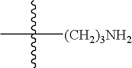 | CH₃ | H | 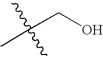 |
| TII7 | 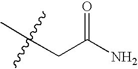 | 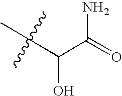 | CH₃ | 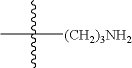 | CH₃ | H | 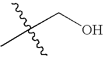 |
| TII8 | 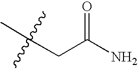 | 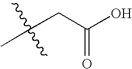 | CH₃ | CH₃ | CH₃ | H | 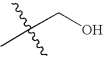 |
| TII9 | 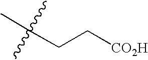 | 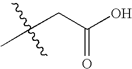 | CH₃ | 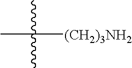 | CH₃ | H | 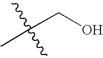 |
| TII10 | 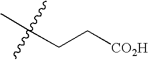 | 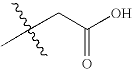 | H | CH₃ | CH₃ | H | 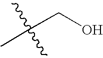 |
| TII11 | 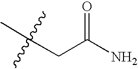 | 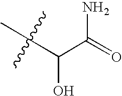 | H | CH₃ | CH₃ | H | 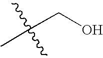 |
| TII12 | 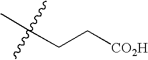 | 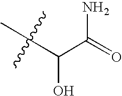 | H | CH₃ | CH₃ | H | 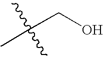 |
| TII13 | 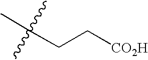 | 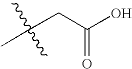 | CH₃ | CH₃ | CH₃ | H | 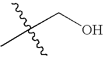 |
| TII14 | 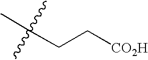 | 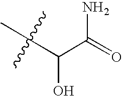 | CH₃ | 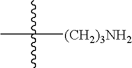 | CH₃ | H | 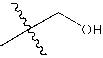 |
| TII15 | 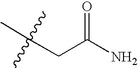 | 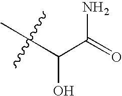 | CH₃ | CH₃ | CH₃ | H | 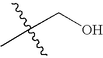 |
| TII16 | 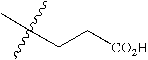 | 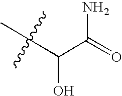 | CH₃ | CH₃ | CH₃ | H | 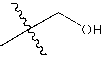 |
| TII17 | 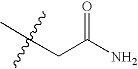 | 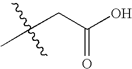 | H | 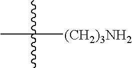 | CH₃ | H | 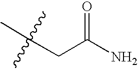 |
| TII18 | 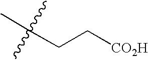 | 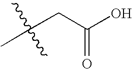 | H | 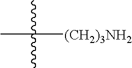 | CH₃ | H | 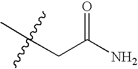 |
| TII19 | 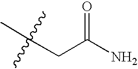 | 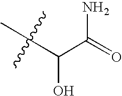 | H | 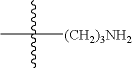 | CH₃ | H | 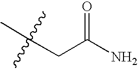 |
| TII20 | 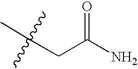 | 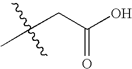 | CH₃ | 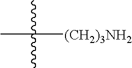 | CH₃ | H | 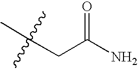 |
| TII21 | 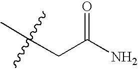 | 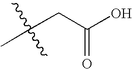 | H | CH₃ | CH₃ | H | 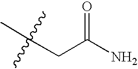 |
| TII22 | 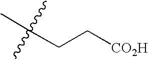 | 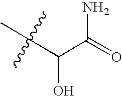 | H | 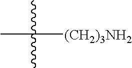 | CH₃ | H | 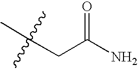 |
| TII23 | 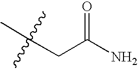 | 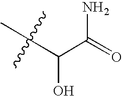 | CH₃ | 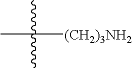 | CH₃ | H | 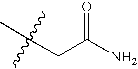 |
| TII24 | 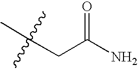 | 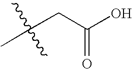 | CH₃ | CH₃ | CH₃ | H | 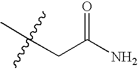 |
| TII25 | 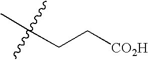 | 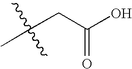 | CH₃ | 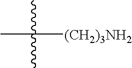 | CH₃ | H | 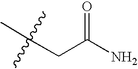 |
| TII26 | 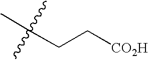 | 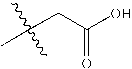 | H | CH₃ | CH₃ | H | 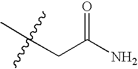 |
| TII27 | 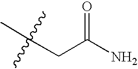 | 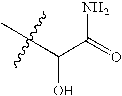 | H | CH₃ | CH₃ | H | 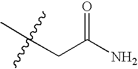 |
| TII28 | 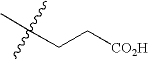 | 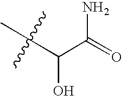 | H | CH₃ | CH₃ | H | 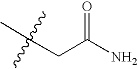 |
| TII29 | 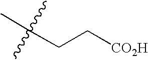 | 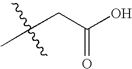 | CH₃ | CH₃ | CH₃ | H | 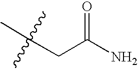 |
| TII30 | 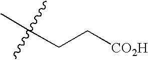 | 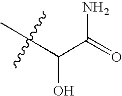 | CH₃ | 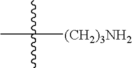 | CH₃ | H | 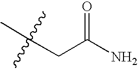 |
| TII31 | 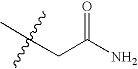 | 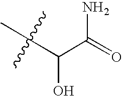 | CH₃ | CH₃ | CH₃ | H | 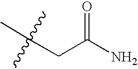 |
| TII32 | 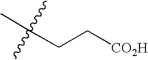 | 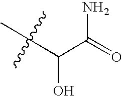 | CH₃ | CH₃ | CH₃ | H | 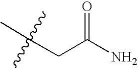 |
| TII33 | 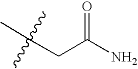 | 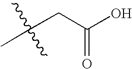 | H | 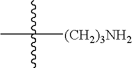 | 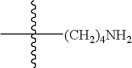 | H | 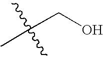 |
| TII34 | 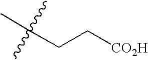 | 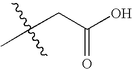 | H | 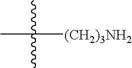 | 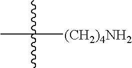 | H | 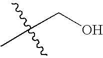 |
| TII35 | 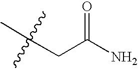 | 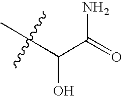 | H | 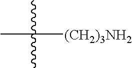 | 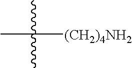 | H | 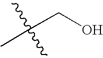 |
| TII36 | 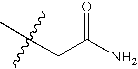 | 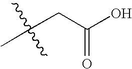 | CH₃ | 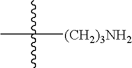 | 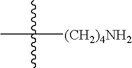 | H | 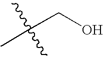 |
| TII37 | 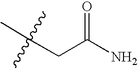 | 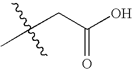 | H | CH₃ | 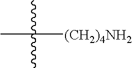 | H | 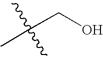 |
| TII38 | 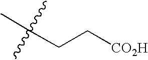 | 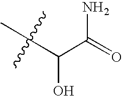 | H | 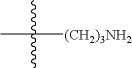 | 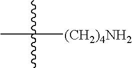 | H | 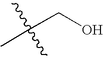 |
| TII39 | 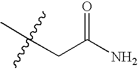 | 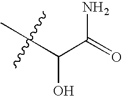 | CH₃ | 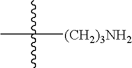 | 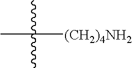 | H | 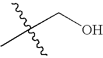 |
| TII40 | 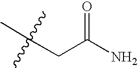 | 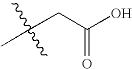 | CH₃ | CH₃ | 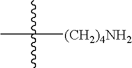 | H | 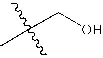 |
| TII41 | 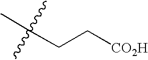 | 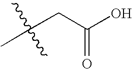 | CH₃ | 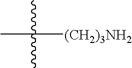 | 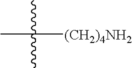 | H | 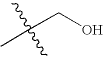 |
| TII42 | 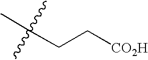 | 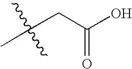 | H | CH₃ | 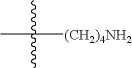 | H | 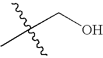 |
| TII43 | 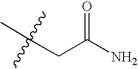 | 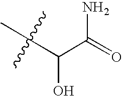 | H | CH₃ | 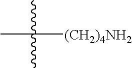 | H | 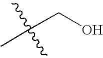 |
| TII44 | 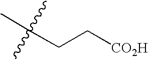 | 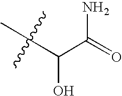 | H | CH₃ | 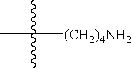 | H | 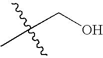 |
| TII45 | 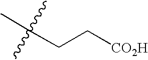 | 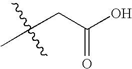 | CH₃ | CH₃ | 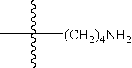 | H | 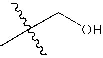 |
| TII46 | 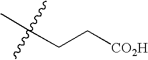 | 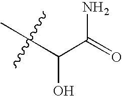 | CH₃ | 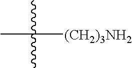 | 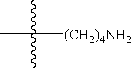 | H | 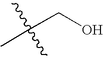 |
| TII47 | 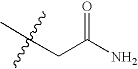 | 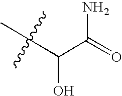 | CH₃ | CH₃ | 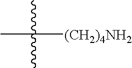 | H | 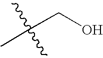 |
| TII48 | 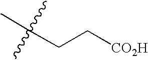 | 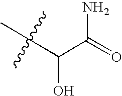 | CH₃ | CH₃ | 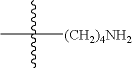 | H | 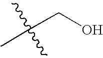 |
| TII49 | 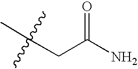 | 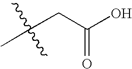 | H | 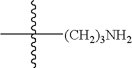 | CH₃ | OMe | 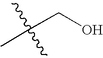 |
| TII50 | 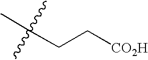 | 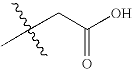 | H | 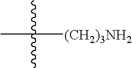 | CH₃ | OMe | 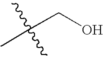 |
| TII51 | 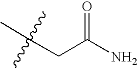 | 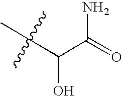 | H | 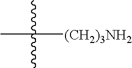 | CH₃ | OMe | 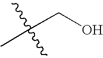 |
| TII52 | 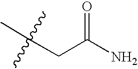 | 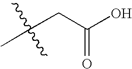 | CH₃ | 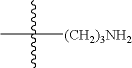 | CH₃ | OMe | 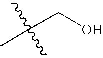 |
| TII53 | 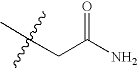 | 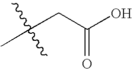 | H | CH₃ | CH₃ | OMe | 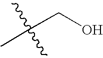 |
| TII54 | 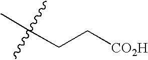 | 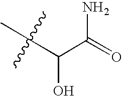 | H | 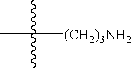 | CH₃ | OMe | 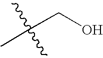 |
| TII55 | 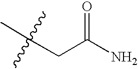 | 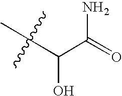 | CH₃ | 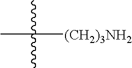 | CH₃ | OMe | 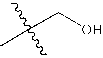 |
| TII56 | 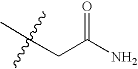 | 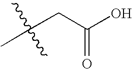 | CH₃ | CH₃ | CH₃ | OMe | 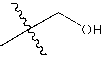 |
| TII57 | 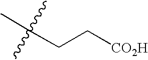 | 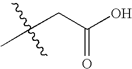 | CH₃ | 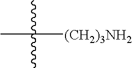 | CH₃ | OMe | 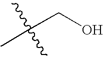 |
| TII58 | 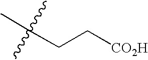 | 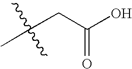 | H | CH₃ | CH₃ | OMe | 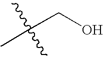 |
| TII59 | 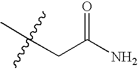 | 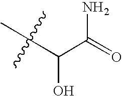 | H | CH₃ | CH₃ | OMe | 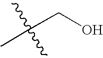 |
| TII60 | 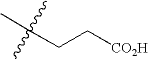 | 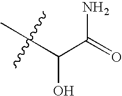 | H | CH₃ | CH₃ | OMe | 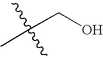 |
| TII61 | 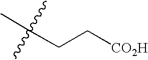 | 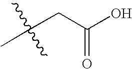 | CH₃ | CH₃ | CH₃ | OMe | 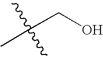 |
| TII62 | 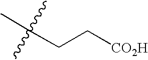 | 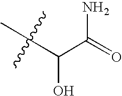 | CH₃ | 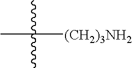 | CH₃ | OMe | 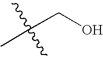 |
| TII63 | 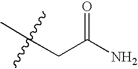 | 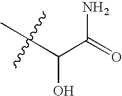 | CH₃ | CH₃ | CH₃ | OMe | 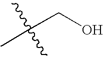 |
| TII64 | 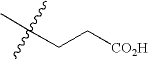 | 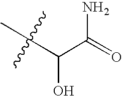 | CH₃ | CH₃ | CH₃ | OMe | 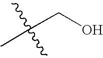 |
| TII65 | 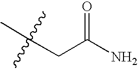 | 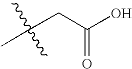 | H | 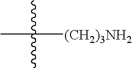 | 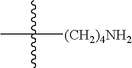 | H | 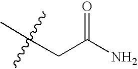 |
| TII66 | 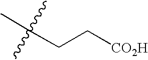 | 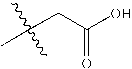 | H | 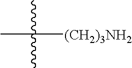 | 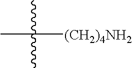 | H | 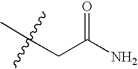 |
| TII67 | 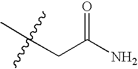 | 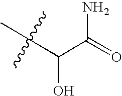 | H | 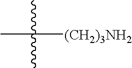 | 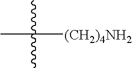 | H | 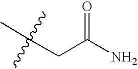 |
| TII68 | 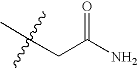 | 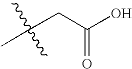 | CH₃ | 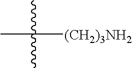 | 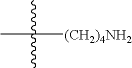 | H | 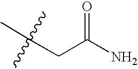 |
| TII69 | 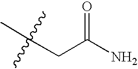 | 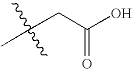 | H | CH₃ | 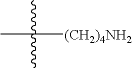 | H | 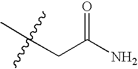 |
| TII70 | 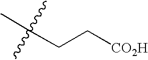 | 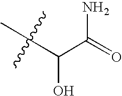 | H | 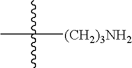 | 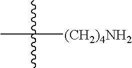 | H | 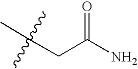 |
| TII71 | 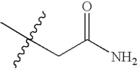 | 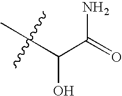 | CH₃ | 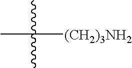 | 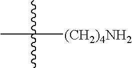 | H | 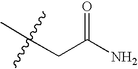 |
| TII72 | 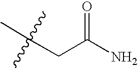 | 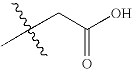 | CH₃ | CH₃ | 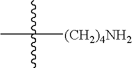 | H | 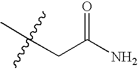 |
| TII73 | 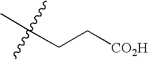 | 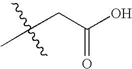 | CH₃ | 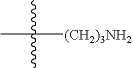 | 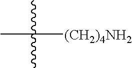 | H | 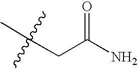 |
| TII74 | 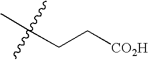 | 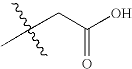 | H | CH₃ | 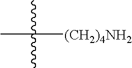 | H | 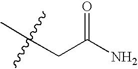 |
| TII75 | 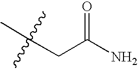 | 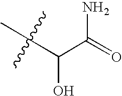 | H | CH₃ | 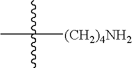 | H | 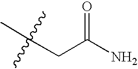 |
| TII76 | 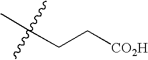 | 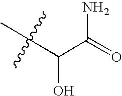 | H | CH₃ | 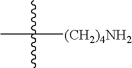 | H | 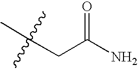 |
| TII77 | 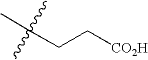 | 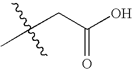 | CH₃ | CH₃ | 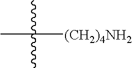 | H | 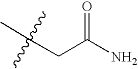 |
| TII78 | 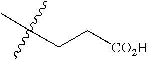 | 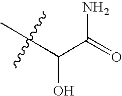 | CH₃ | 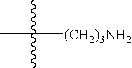 | 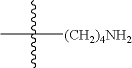 | H | 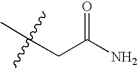 |
| TII79 | 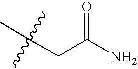 | 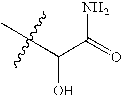 | CH₃ | CH₃ | 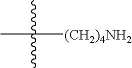 | H | 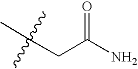 |
| TII80 | 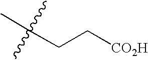 | 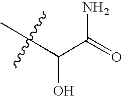 | CH₃ | CH₃ | 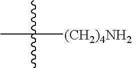 | H | 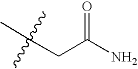 |
| TII81 | 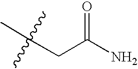 | 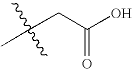 | H | 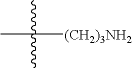 | CH₃ | OMe | 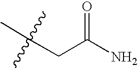 |
| TII82 | 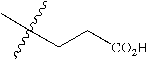 | 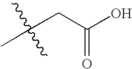 | H | 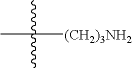 | CH₃ | OMe | 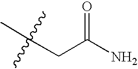 |
| TII83 | 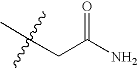 | 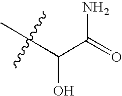 | H | 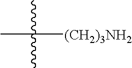 | CH₃ | OMe | 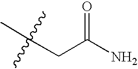 |
| TII84 | 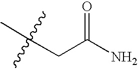 | 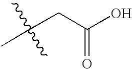 | CH₃ | 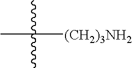 | CH₃ | OMe | 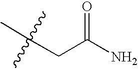 |
| TII85 | 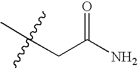 | 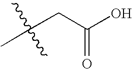 | H | CH₃ | CH₃ | OMe | 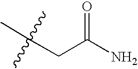 |
| TII86 | 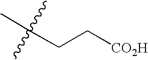 | 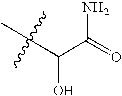 | H | 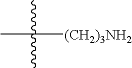 | CH₃ | OMe | 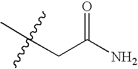 |
| TII87 | 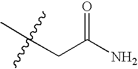 | 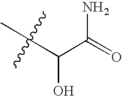 | CH₃ | 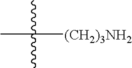 | CH₃ | OMe | 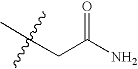 |
| TII88 | 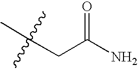 | 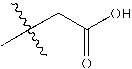 | CH₃ | CH₃ | CH₃ | OMe | 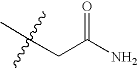 |
| TII89 | 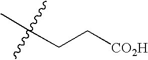 | 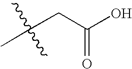 | CH₃ | 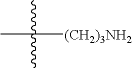 | CH₃ | OMe | 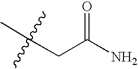 |
| TII90 | 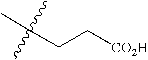 | 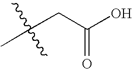 | H | CH₃ | CH₃ | OMe | 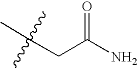 |
| TII91 | 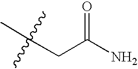 | 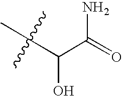 | H | CH₃ | CH₃ | OMe | 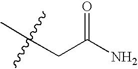 |
| TII92 | 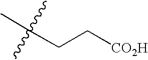 | 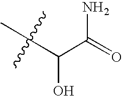 | H | CH₃ | CH₃ | OMe | 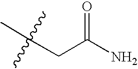 |
| TII93 | 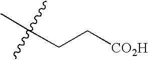 | 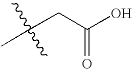 | CH₃ | CH₃ | CH₃ | OMe | 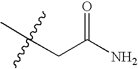 |
| TII94 | 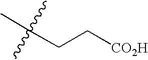 | 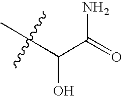 | CH₃ | 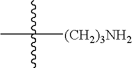 | CH₃ | OMe | 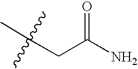 |
| TII95 | 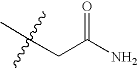 | 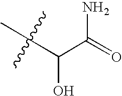 | CH₃ | CH₃ | CH₃ | OMe | 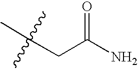 |
| TII96 | 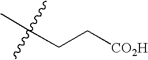 | 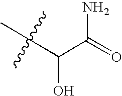 | CH₃ | CH₃ | CH₃ | OMe | 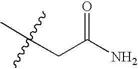 |
| TII97 | 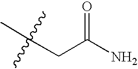 | 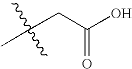 | H | 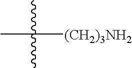 | 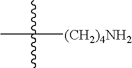 | OMe | 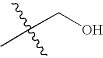 |
| TII98 | 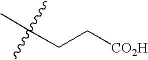 | 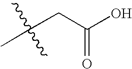 | H | 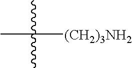 | 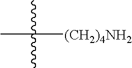 | OMe | 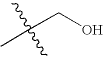 |
| TII99 | 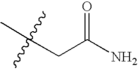 | 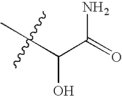 | H | 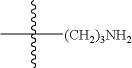 | 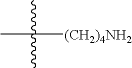 | OMe | 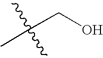 |
| TII100 | 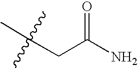 | 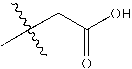 | CH₃ | 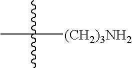 | 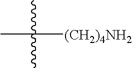 | OMe | 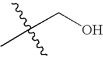 |
| TII101 | 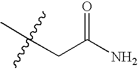 | 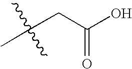 | H | CH₃ | 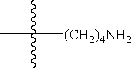 | OMe | 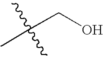 |
| TII102 | 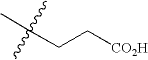 | 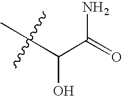 | H | 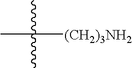 | 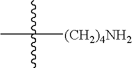 | OMe | 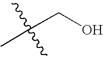 |
| TII103 | 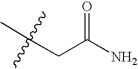 | 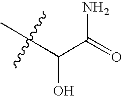 | CH₃ | 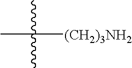 | 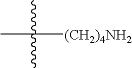 | OMe | 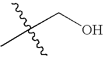 |
| TII104 | 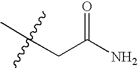 | 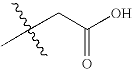 | CH₃ | CH₃ | 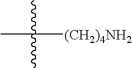 | OMe | 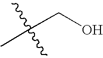 |
| TII105 | 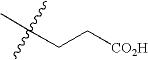 | 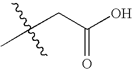 | CH₃ | 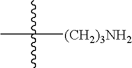 | 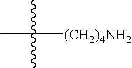 | OMe | 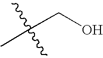 |
| TII106 | 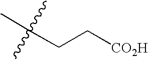 | 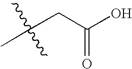 | H | CH₃ | 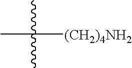 | OMe | 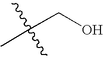 |
| TII107 | 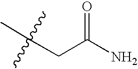 | 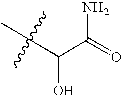 | H | CH₃ | 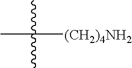 | OMe | 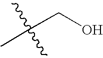 |
| TII108 | 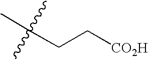 | 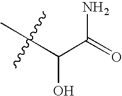 | H | CH₃ | 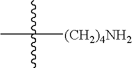 | OMe | 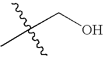 |
| TII109 | 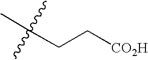 | 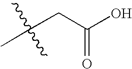 | CH₃ | CH₃ | 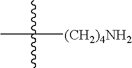 | OMe | 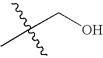 |
| TII110 | 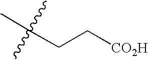 | 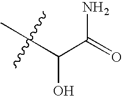 | CH₃ | 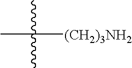 | 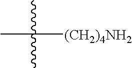 | OMe | 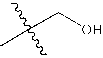 |
| TII111 | 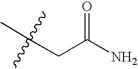 | 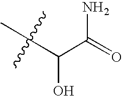 | CH₃ | CH₃ | 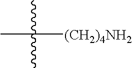 | OMe | 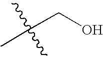 |
| TII112 | 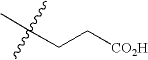 | 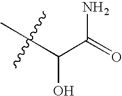 | CH₃ | CH₃ | 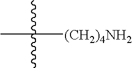 | OMe | 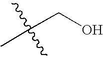 |
| TII113 | 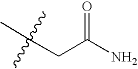 | 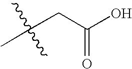 | H | 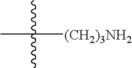 | 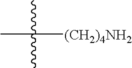 | OMe | 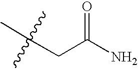 |
| TII114 | 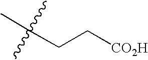 | 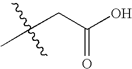 | H | 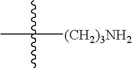 | 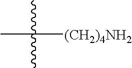 | OMe | 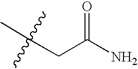 |
| TII115 | 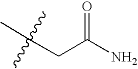 | 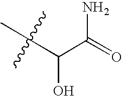 | H | 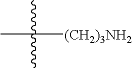 | 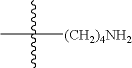 | OMe | 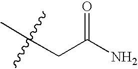 |
| TII116 | 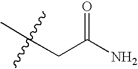 | 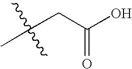 | CH₃ | 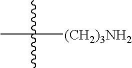 | 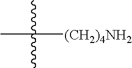 | OMe | 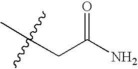 |
| TII117 | 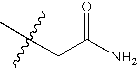 | 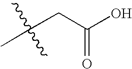 | H | CH₃ | 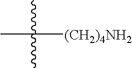 | OMe | 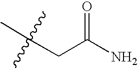 |
| TII118 | 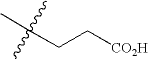 | 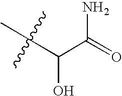 | H | 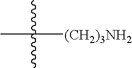 | 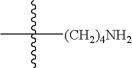 | OMe | 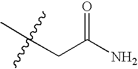 |
| TII119 | 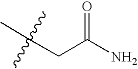 | 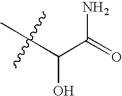 | CH₃ | 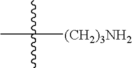 | 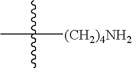 | OMe | 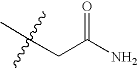 |
| TII120 | 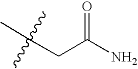 | 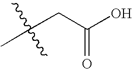 | CH₃ | CH₃ | 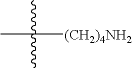 | OMe | 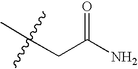 |
| TII121 | 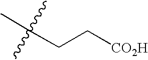 | 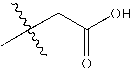 | CH₃ | 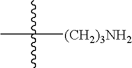 | 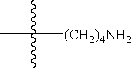 | OMe | 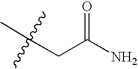 |
| TII122 | 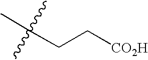 | 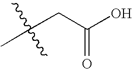 | H | CH₃ | 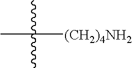 | OMe | 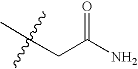 |
| TII123 | 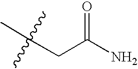 | 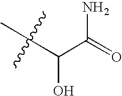 | H | CH₃ | 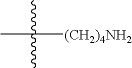 | OMe | 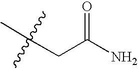 |
| TII124 | 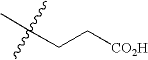 | 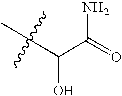 | H | CH₃ | 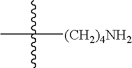 | OMe | 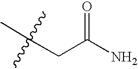 |
| TII125 | 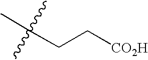 | 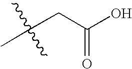 | CH₃ | CH₃ | 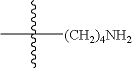 | OMe | 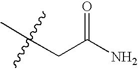 |
| TII126 | 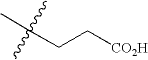 | 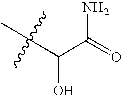 | CH₃ | 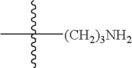 | 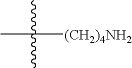 | OMe | 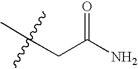 |
| TII127 | 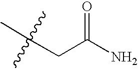 | 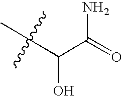 | CH₃ | CH₃ | 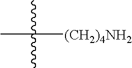 | OMe | 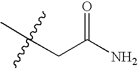 |
| TII128 | 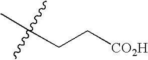 | 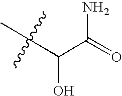 | CH₃ | CH₃ | 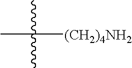 | OMe | 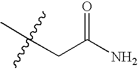 |

In another embodiment of the invention,
R² is 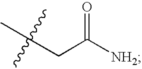
R³ is 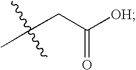
R⁹ is 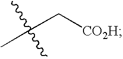
R²*, R⁵, R⁵*, R⁸*, and R¹¹* are each H; and
R⁶ is 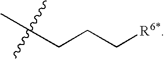
This embodiment gives a compound of Formula III. 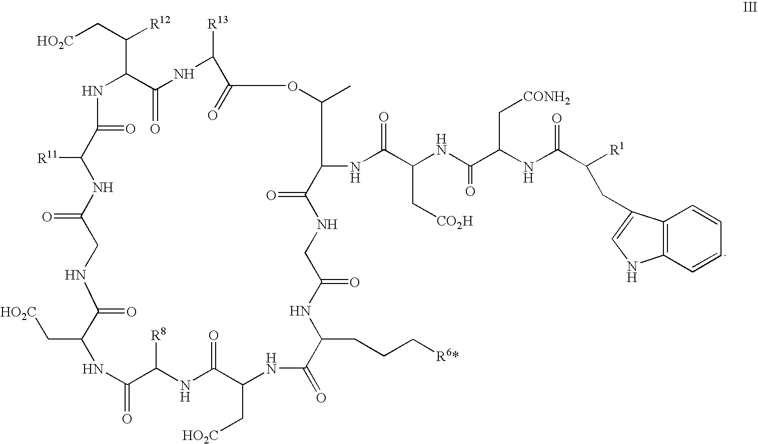
wherein R¹, R⁶*, R⁸, R¹¹, R¹² and R¹³ are as previously defined.

Table III provides exemplary compounds of Formula III.

TABLE III
Compounds of Formula III

| | |
|---|---|
| | III |
| 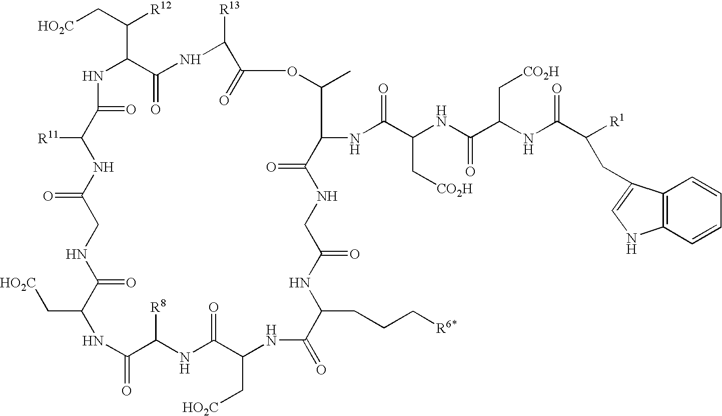 | |

| # | R⁸ | R¹¹ | R¹² | R¹³ |
|---|---|---|---|---|
| TIII1 | CH₃ | 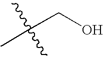 | CH₃ | 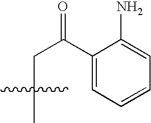 |
| TIII2 | CH₃ | 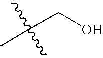 | CH₃ | 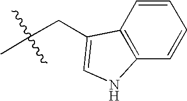 |
| TIII3 | CH₃ | 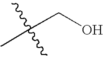 | CH₃ | 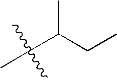 |
| TIII4 | CH₃ | 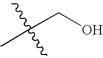 | CH₃ | 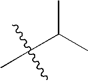 |
| TIII5 | CH₃ | 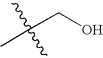 | H | 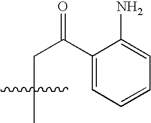 |
| TIII6 | CH₃ | 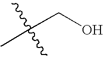 | H | 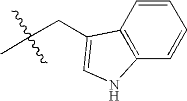 |
| TIII7 | CH₃ | 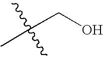 | H | 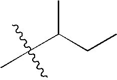 |
| TIII8 | CH₃ | 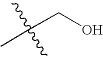 | H | 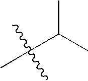 |
| TIII9 | 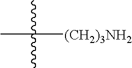 | 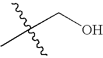 | CH₃ | 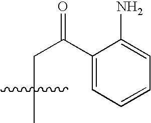 |
| TIII10 | 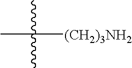 | 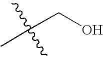 | CH₃ | 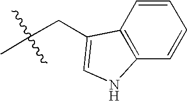 |
| TIII11 | 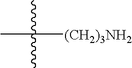 | 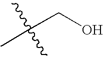 | CH₃ | 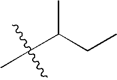 |
| TIII12 | 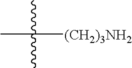 | 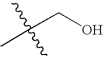 | CH₃ | 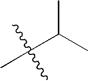 |
| TIII13 | 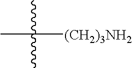 | 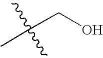 | H | 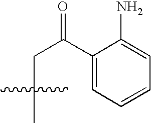 |
| TIII14 | 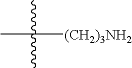 | 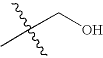 | H | 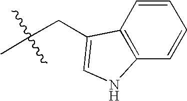 |
| TIII15 | 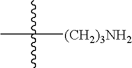 | 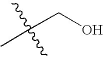 | H | 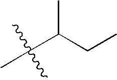 |
| TIII16 | 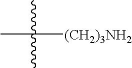 | 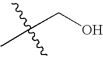 | H | 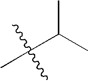 |
| TIII17 | 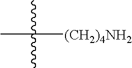 | 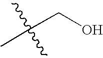 | CH₃ | 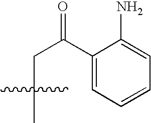 |
| TIII18 | 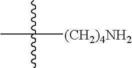 | 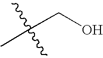 | CH₃ | 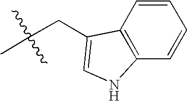 |
| TIII19 | 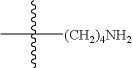 | 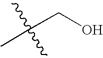 | CH₃ | 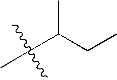 |
| TIII20 | 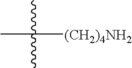 | 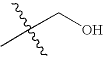 | CH₃ | 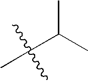 |
| TIII21 | 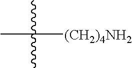 | 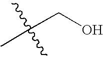 | H | 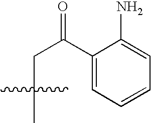 |
| TIII22 | 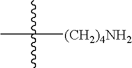 | 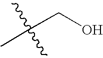 | H | 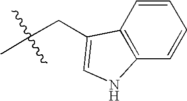 |
| TIII23 | 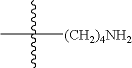 | 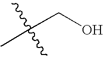 | H | 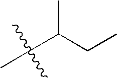 |
| TIII24 | 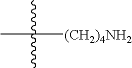 | 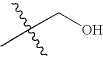 | H | 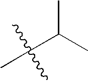 |
| TIII25 | 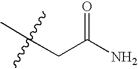 | 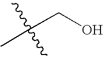 | CH₃ | 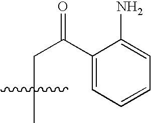 |
| TIII26 | 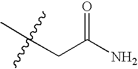 | 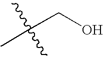 | CH₃ | 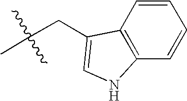 |
| TIII27 | 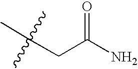 | 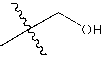 | CH₃ | 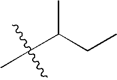 |
| TIII28 | 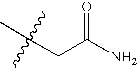 | 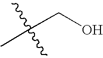 | CH₃ | 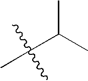 |
| TIII29 | 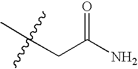 | 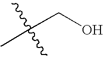 | H | 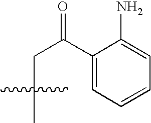 |
| TIII30 | 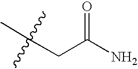 | 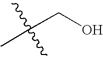 | H | 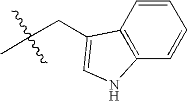 |
| TIII31 | 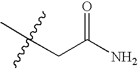 | 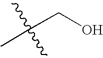 | H | 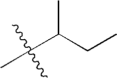 |
| TIII32 | 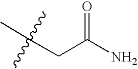 | 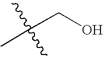 | H | 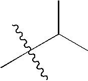 |
| TIII33 | 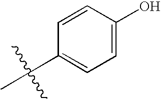 | 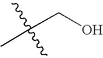 | CH₃ | 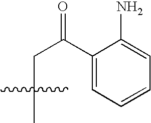 |
| TIII34 | 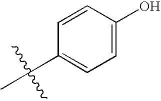 | 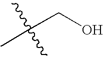 | CH₃ | 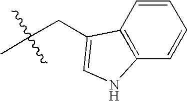 |
| TIII35 | 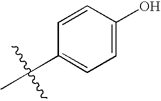 | 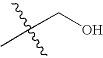 | CH₃ | 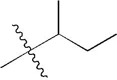 |
| TIII36 | 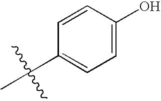 | 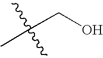 | CH₃ | 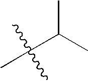 |
| TIII37 | 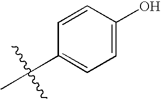 | 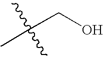 | H | 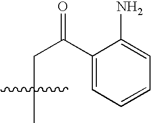 |
| TIII38 | 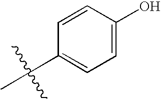 | 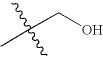 | H | 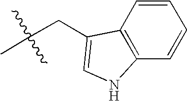 |
| TIII39 | 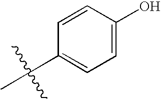 | 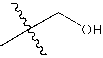 | H | 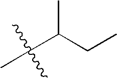 |
| TIII40 | 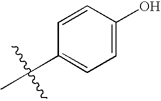 | 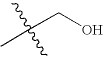 | H | 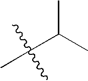 |
| TIII41 | 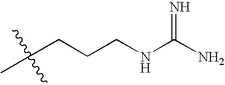 | 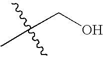 | CH₃ | 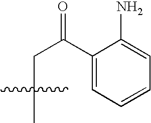 |
| TIII42 | 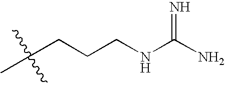 | 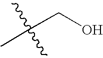 | CH₃ | 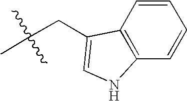 |
| TIII43 | 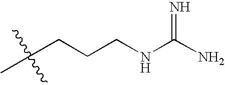 | 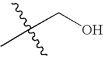 | CH₃ | 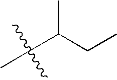 |
| TIII44 | 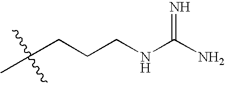 | 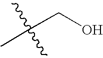 | CH₃ | 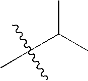 |
| TIII45 | 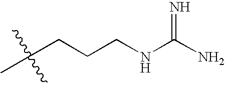 | 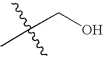 | H | 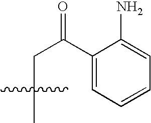 |
| TIII46 | 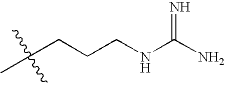 | 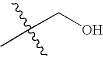 | H | 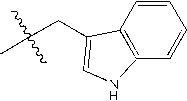 |
| TIII47 | 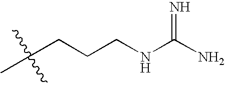 | 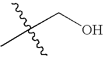 | H | 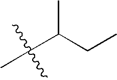 |
| TIII48 | 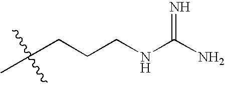 | 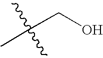 | H | 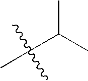 |
| TIII49 | 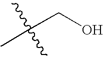 | 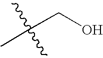 | CH₃ | 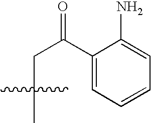 |
| TIII50 | 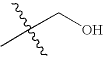 | 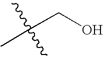 | CH₃ | 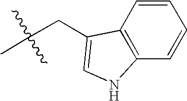 |
| TIII51 | 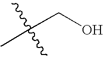 | 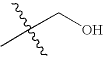 | CH₃ | 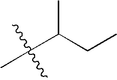 |
| TIII52 | 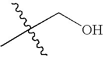 | 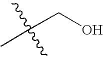 | CH₃ | 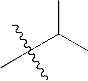 |
| TIII53 | 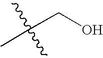 | 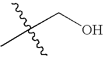 | H | 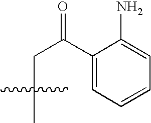 |
| TIII54 | 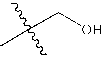 | 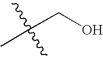 | H | 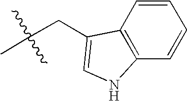 |
| TIII55 | 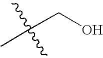 | 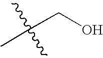 | H | 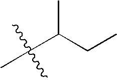 |
| TIII56 | 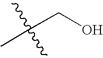 | 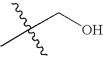 | H | 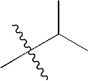 |
| TIII57 | 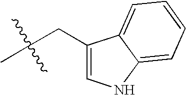 | 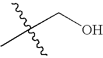 | CH₃ | 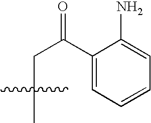 |
| TIII58 | 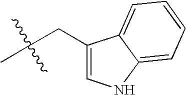 | 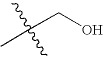 | CH₃ | 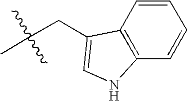 |
| TIII59 | 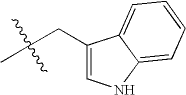 | 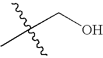 | CH₃ | 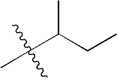 |
| TIII60 | 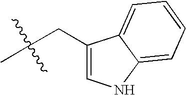 | 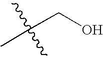 | CH₃ | 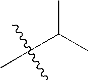 |
| TIII61 | 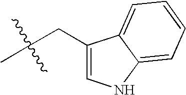 | 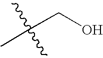 | H | 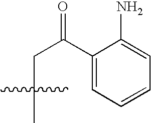 |
| TIII62 | 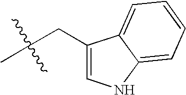 | 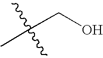 | H | 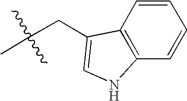 |
| TIII63 | 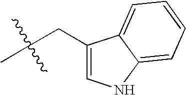 | 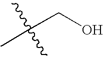 | H | 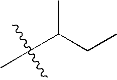 |
| TIII64 | 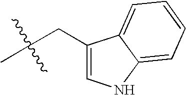 | 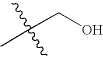 | H | 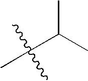 |
| TIII65 | 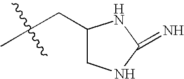 | 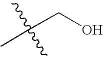 | CH₃ | 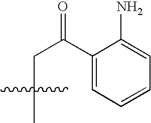 |
| TIII66 | 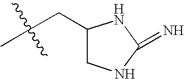 | 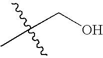 | CH₃ | 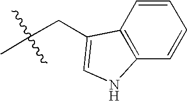 |
| TIII67 | 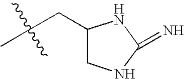 | 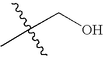 | CH₃ | 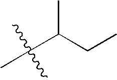 |
| TIII68 | 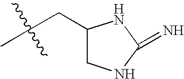 | 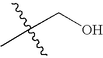 | CH₃ | 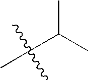 |
| TIII69 | 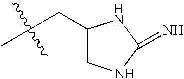 | 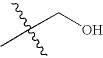 | H | 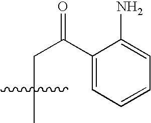 |
| TIII70 | 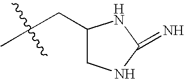 | 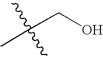 | H | 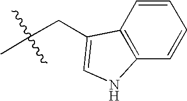 |
| TIII71 | 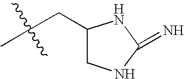 | 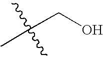 | H | 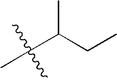 |
| TIII72 | 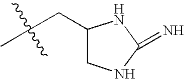 | 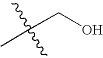 | H | 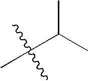 |
| TIII73 | 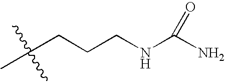 | 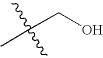 | CH₃ | 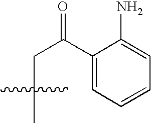 |
| TIII74 | 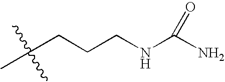 | 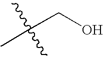 | CH₃ | 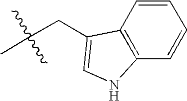 |
| TIII75 | 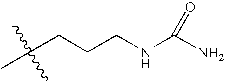 | 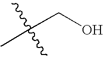 | CH₃ | 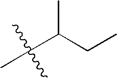 |
| TIII76 | 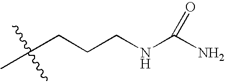 | 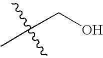 | CH₃ | 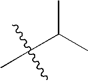 |
| TIII77 | 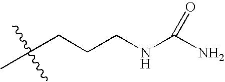 | 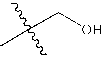 | H | 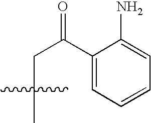 |
| TIII78 | 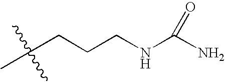 | 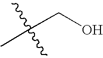 | H | 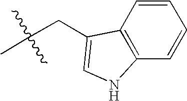 |
| TIII79 | 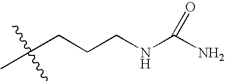 | 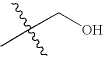 | H | 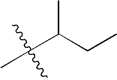 |
| TIII80 | 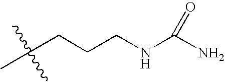 | 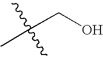 | H | 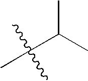 |
| TIII81 | 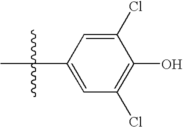 | 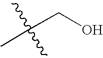 | CH₃ | 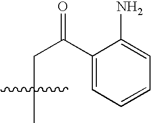 |
| TIII82 | 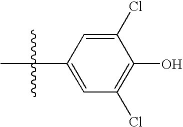 | 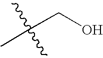 | CH₃ | 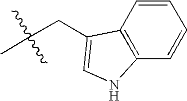 |
| TIII83 | 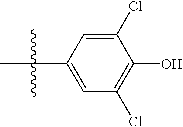 | 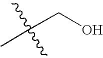 | CH₃ | 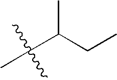 |
| TIII84 | 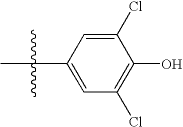 | 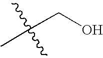 | CH₃ | 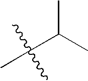 |
| TIII85 | 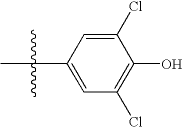 | 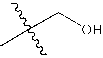 | H | 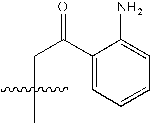 |
| TIII86 | 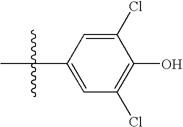 | 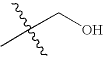 | H | 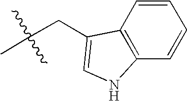 |
| TIII87 | 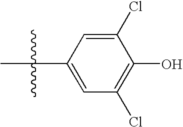 | 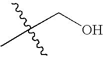 | H | 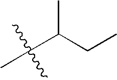 |
| TIII88 | 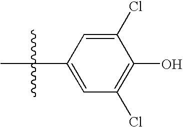 | 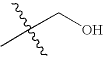 | H | 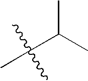 |
| TIII89 | CH₃ | CH₃ | CH₃ | 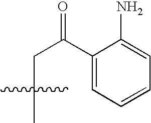 |
| TIII90 | CH₃ | CH₃ | CH₃ | 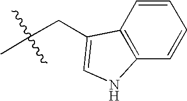 |
| TIII91 | CH₃ | CH₃ | CH₃ | 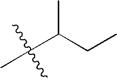 |
| TIII92 | CH₃ | CH₃ | CH₃ | 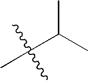 |
| TIII93 | CH₃ | CH₃ | H | 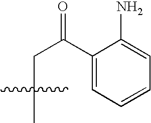 |
| TIII94 | CH₃ | CH₃ | H | 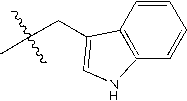 |
| TIII95 | CH₃ | CH₃ | H | 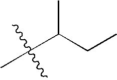 |
| TIII96 | CH₃ | CH₃ | H | 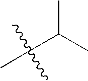 |
| TIII97 | CH₃ | 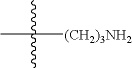 | CH₃ | 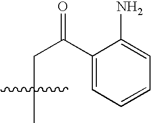 |
| TIII98 | CH₃ | 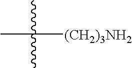 | CH₃ | 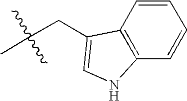 |
| TIII99 | CH₃ | 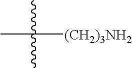 | CH₃ | 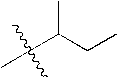 |
| TIII100 | CH₃ | 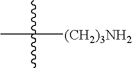 | CH₃ | 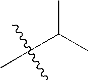 |
| TIII101 | CH₃ | 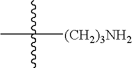 | H | 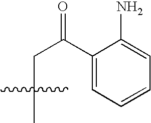 |
| TIII102 | CH₃ | 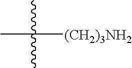 | H | 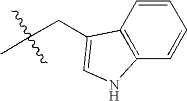 |
| TIII103 | CH₃ | 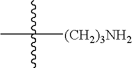 | H | 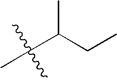 |
| TIII104 | CH₃ | 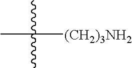 | H | 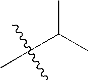 |
| TIII105 | CH₃ | 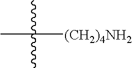 | CH₃ | 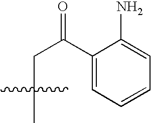 |
| TIII106 | CH₃ | 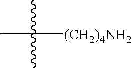 | CH₃ | 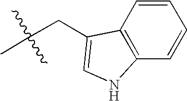 |
| TIII107 | CH₃ | 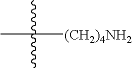 | CH₃ | 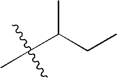 |
| TIII108 | CH₃ | 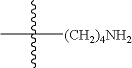 | CH₃ | 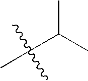 |
| TIII109 | CH₃ | 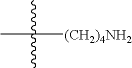 | H | 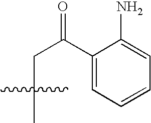 |
| TIII110 | CH₃ | 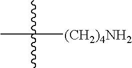 | H | 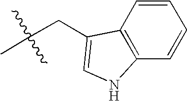 |
| TIII111 | CH₃ | 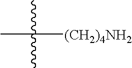 | H | 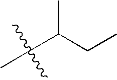 |
| TIII112 | CH₃ | 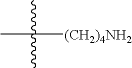 | H | 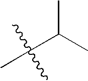 |
| TIII113 | CH₃ | 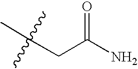 | CH₃ | 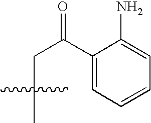 |
| TIII114 | CH₃ | 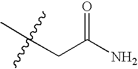 | CH₃ | 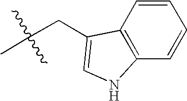 |
| TIII115 | CH₃ | 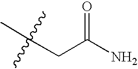 | CH₃ | 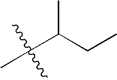 |
| TIII116 | CH₃ | 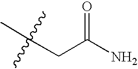 | CH₃ | 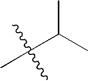 |
| TIII117 | CH₃ | 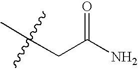 | H | 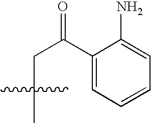 |
| TIII118 | CH₃ | 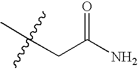 | H | 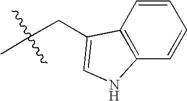 |
| TIII119 | CH₃ | 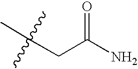 | H | 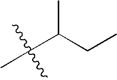 |
| TIII120 | CH₃ | 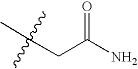 | H | 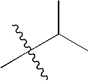 |
| TIII121 | CH₃ | 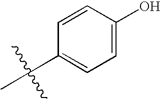 | CH₃ | 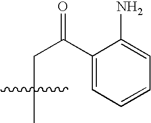 |
| TIII122 | CH₃ | 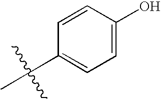 | CH₃ | 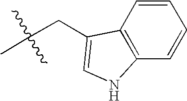 |
| TIII123 | CH₃ | 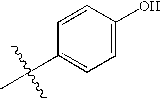 | CH₃ | 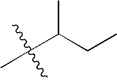 |
| TIII124 | CH₃ | 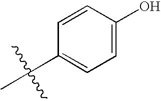 | CH₃ | 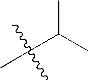 |
| TIII125 | CH₃ | 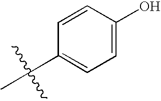 | H | 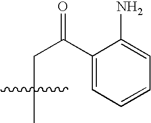 |
| TIII126 | CH₃ | 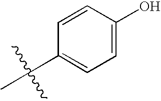 | H | 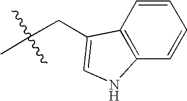 |
| TIII127 | CH₃ | 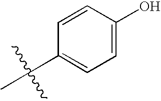 | H | 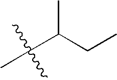 |
| TIII128 | CH₃ | 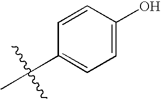 | H | 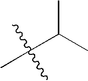 |
| TIII129 | CH₃ | 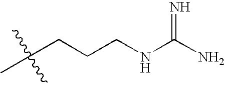 | CH₃ | 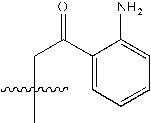 |
| TIII130 | CH₃ | 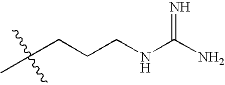 | CH₃ | 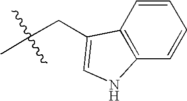 |
| TIII131 | CH₃ | 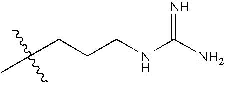 | CH₃ | 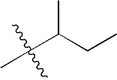 |
| TIII132 | CH₃ | 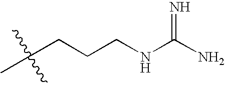 | CH₃ | 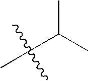 |
| TIII133 | CH₃ | 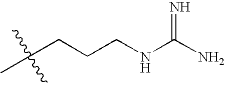 | H | 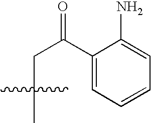 |
| TIII134 | CH₃ | 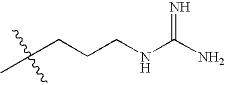 | H | 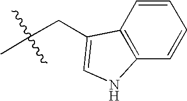 |
| TIII135 | CH₃ | 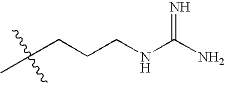 | H | 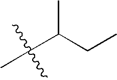 |
| TIII136 | CH₃ | 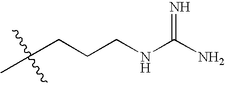 | H | 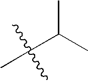 |
| TIII137 | CH₃ | 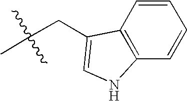 | CH₃ | 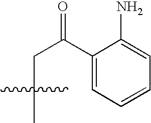 |
| TIII138 | CH₃ | 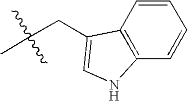 | CH₃ | 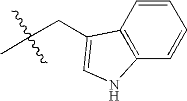 |
| TIII139 | CH₃ | 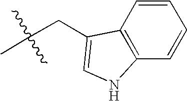 | CH₃ | 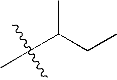 |
| TIII140 | CH₃ | 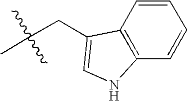 | CH₃ | 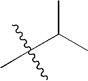 |
| TIII141 | CH₃ | 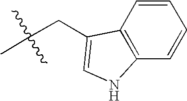 | H | 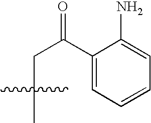 |
| TIII142 | CH₃ | 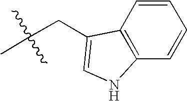 | H | 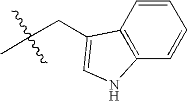 |
| TIII143 | CH₃ | 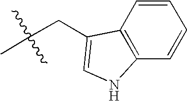 | H | 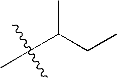 |
| TIII144 | CH₃ | 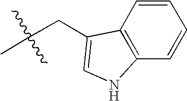 | H | 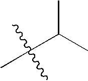 |
| TIII145 | CH₃ | 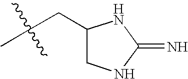 | CH₃ | 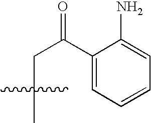 |
| TIII146 | CH₃ | 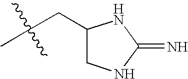 | CH₃ | 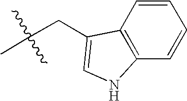 |
| TIII147 | CH₃ | 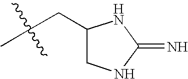 | CH₃ | 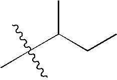 |
| TIII148 | CH₃ | 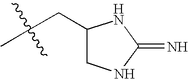 | CH₃ | 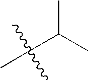 |
| TIII149 | CH₃ | 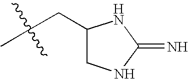 | H | 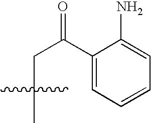 |
| TIII150 | CH₃ | 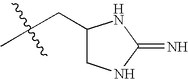 | H | 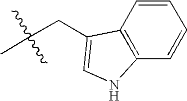 |
| TIII151 | CH₃ | 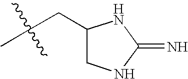 | H | 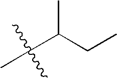 |
| TIII152 | CH₃ | 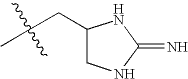 | H | 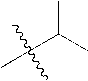 |
| TIII153 | CH₃ | 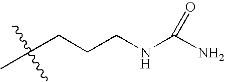 | CH₃ | 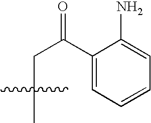 |
| TIII154 | CH₃ | 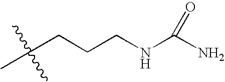 | CH₃ | 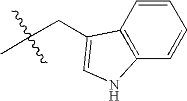 |
| TIII155 | CH₃ | 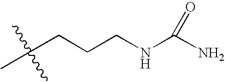 | CH₃ | 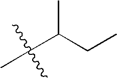 |
| TIII156 | CH₃ | 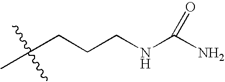 | CH₃ | 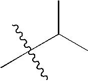 |
| TIII157 | CH₃ | 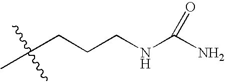 | H | 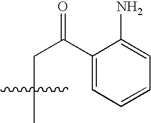 |
| TIII158 | CH₃ | 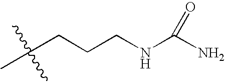 | H | 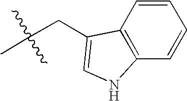 |
| TIII159 | CH₃ | 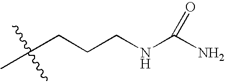 | H | 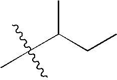 |
| TIII160 | CH₃ | 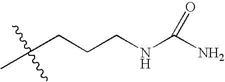 | H | 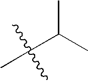 |
| TIII161 | CH₃ | 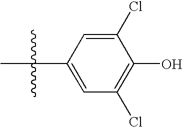 | CH₃ | 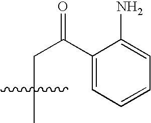 |
| TIII162 | CH₃ | 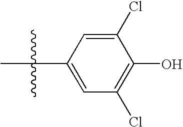 | CH₃ | 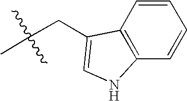 |
| TIII163 | CH₃ | 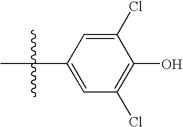 | CH₃ | 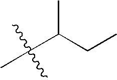 |
| TIII164 | CH₃ | 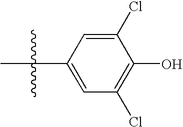 | CH₃ | 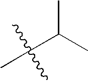 |
| TIII165 | CH₃ | 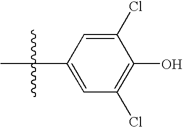 | H | 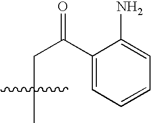 |
| TIII166 | CH₃ | 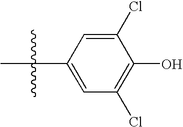 | H | 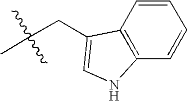 |
| TIII167 | CH₃ | 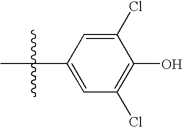 | H | 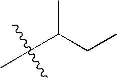 |
| TIII168 | CH₃ | 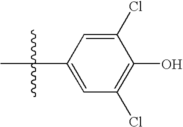 | H | 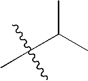 |

In another embodiment of the invention each of R²*, R⁸* and R¹¹* is H. This embodiment gives a compound of Formula IV.

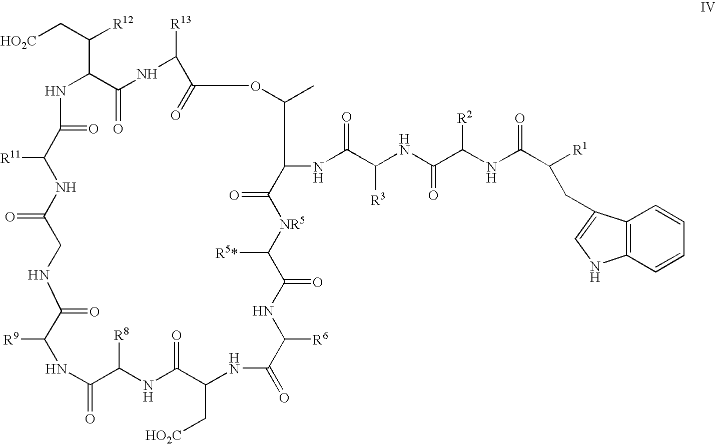

Table IV provides exemplary compounds of Formula IV.

TABLE IV
Compounds of Formula IV

| | |
|---|---|
| | (IV) |
| 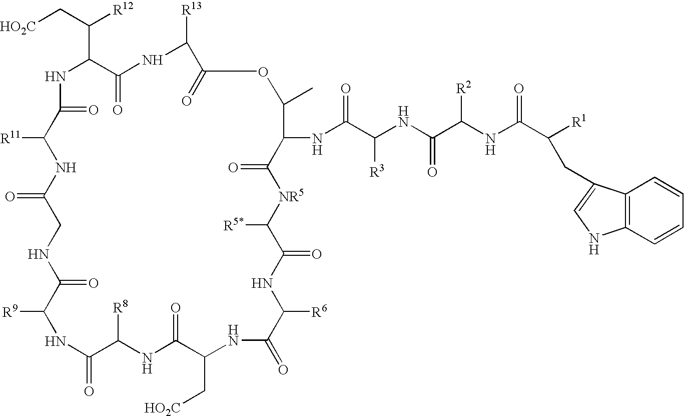 | |

| # | R² | R³ | R⁵ | R^{5*} | R⁶ |
|---|---|---|---|---|---|
| TIV1 | 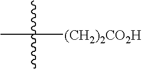 | 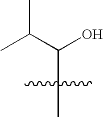 | CH₃ | H | CH₃ |
| TIV2 | 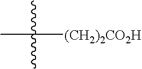 | 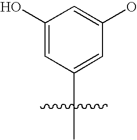 | CH₃ | H | CH₃ |
| TIV3 | 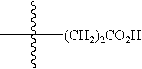 | 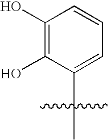 | CH₃ | H | CH₃ |
| TIV4 | 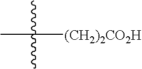 | 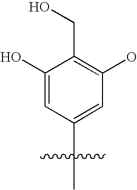 | CH₃ | H | CH₃ |
| TIV5 | 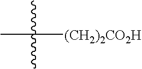 | 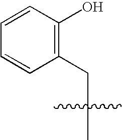 | CH₃ | H | CH₃ |
| TIV6 | 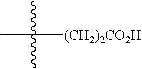 | 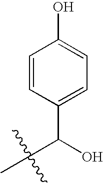 | CH₃ | H | CH₃ |
| TIV7 | 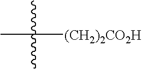 | 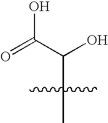 | CH₃ | H | CH₃ |
| TIV8 | 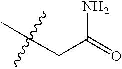 | 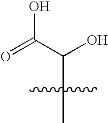 | CH₃ | H | CH₃ |
| TIV9 | 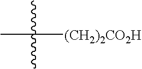 | 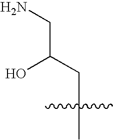 | CH₃ | H | CH₃ |
| TIV10 | 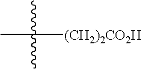 | 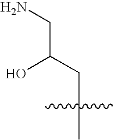 | CH₃ | H | CH₃ |
| TIV11 | 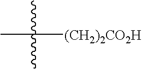 | 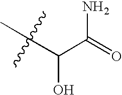 | CH₃ | 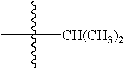 | CH₃ |
| TIV12 | 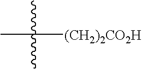 | 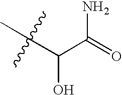 | CH₃ | 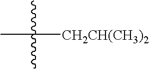 | CH₃ |
| TIV13 | 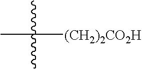 | 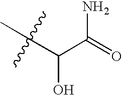 | CH₃ | CH₃ | CH₃ |
| TIV14 | 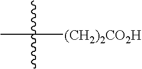 | 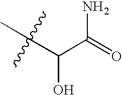 | CH₃ | 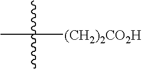 | CH₃ |
| TIV15 | 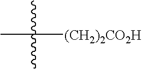 | 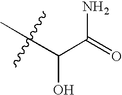 | CH₃ | 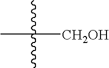 | CH₃ |
| TIV16 | 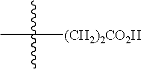 | 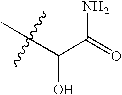 | CH₃ | 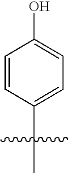 | CH₃ |
| TIV17 | 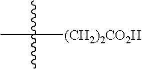 | 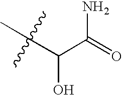 | CH₃ | 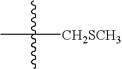 | CH₃ |
| TIV18 | 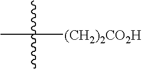 | 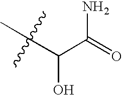 | CH₃ | 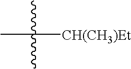 | CH₃ |
| TIV19 | 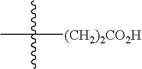 | 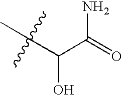 | CH₃ | 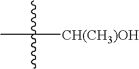 | CH₃ |
| TIV20 | 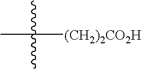 | 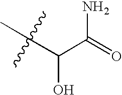 | CH₃ | 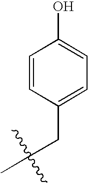 | CH₃ |
| TIV21 | 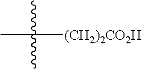 | 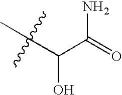 | CH₃ | 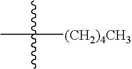 | CH₃ |
| TIV22 | 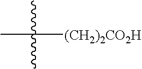 | 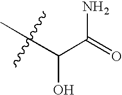 | CH₃ | 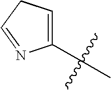 | CH₃ |
| TIV23 | 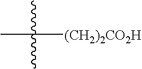 | 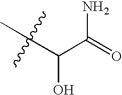 | CH₃ | 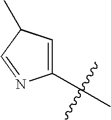 | CH₃ |
| TIV24 | 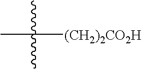 | 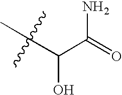 | CH₃ | 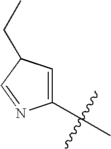 | CH₃ |
| TIV25 | 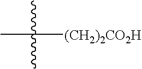 | 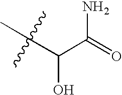 | H | 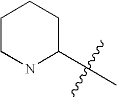 | CH₃ |
| TIV26 | 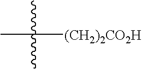 | 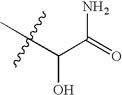 | CH₃ | 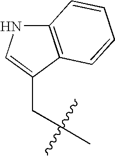 | CH₃ |
| TIV27 | 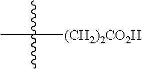 | 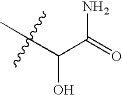 | CH₃ | 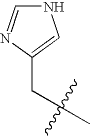 | CH₃ |
| TIV28 | 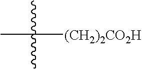 | 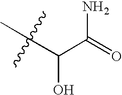 | CH₃ | 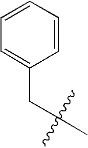 | CH₃ |
| TIV29 | 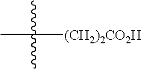 | 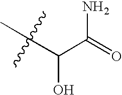 | CH₃ | 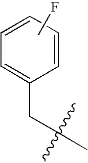 | CH₃ |
| TIV30 | 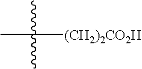 | 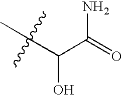 | CH₃ | 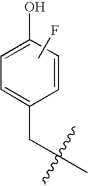 | CH₃ |
| TIV31 | 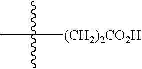 | 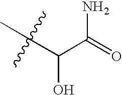 | CH₃ | 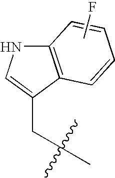 | CH₃ |
| TIV32 | 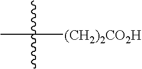 | 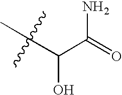 | CH₃ | 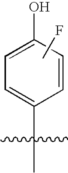 | CH₃ |
| TIV33 | 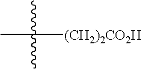 | 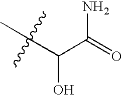 | CH₃ | 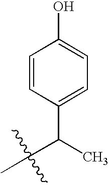 | CH₃ |
| TIV34 | 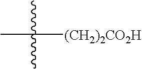 | 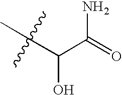 | CH₃ | 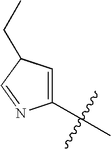 | CH₃ |
| TIV35 | 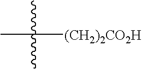 | 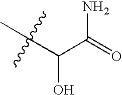 | CH₃ | H | CH₃ |
| TIV36 | 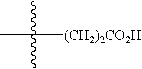 | 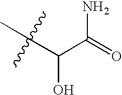 | CH₃ | H | CH₃ |
| TIV37 | 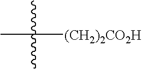 | 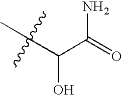 | CH₃ | H | CH₃ |
| TIV38 | 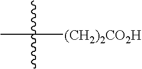 | 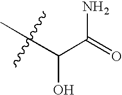 | CH₃ | H | CH₃ |
| TIV39 | 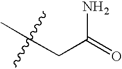 | 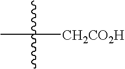 | H | H | 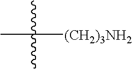 |
| TIV40 | 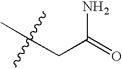 | 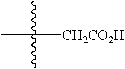 | H | H | 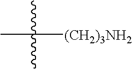 |
| TIV41 | 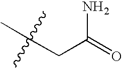 | 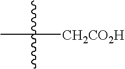 | H | H | 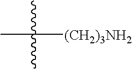 |
| TIV42 | 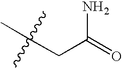 | 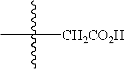 | H | H | 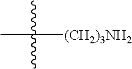 |
| TIV43 | 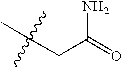 | 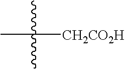 | H | H | 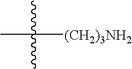 |
| TIV44 | 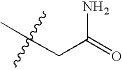 | 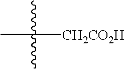 | H | H | 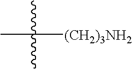 |
| TIV45 | 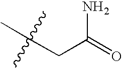 | 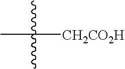 | H | H | 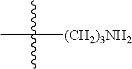 |
| TIV46 | 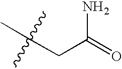 | 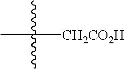 | H | H | 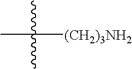 |
| TIV47 | 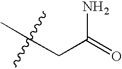 | 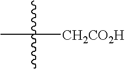 | H | H | 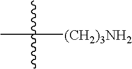 |
| TIV48 | 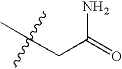 | 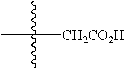 | H | H | 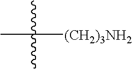 |
| TIV49 | 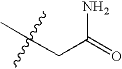 | 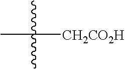 | H | H | 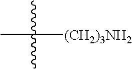 |
| TIV50 | 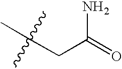 | 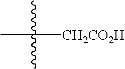 | H | H | 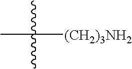 |
| TIV51 | 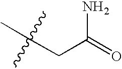 | 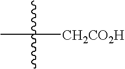 | H | H | CH₃ |
| TIV52 | 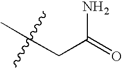 | 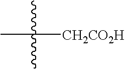 | H | H | CH₃ |
| TIV53 | 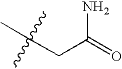 | 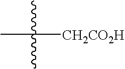 | H | H | CH₃ |
| TIV54 | 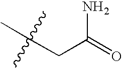 | 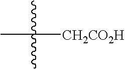 | H | H | CH₃ |
| TIV55 | 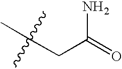 | 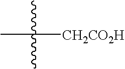 | H | H | CH₃ |
| TIV56 | 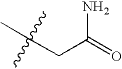 | 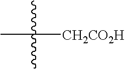 | H | H | CH₃ |
| TIV57 | 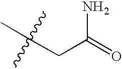 | 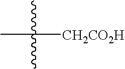 | H | H | CH₃ |
| TIV58 | 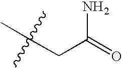 | 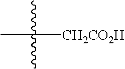 | H | H | CH₃ |
| TIV59 | 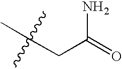 | 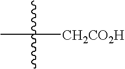 | H | H | CH₃ |
| TIV60 | 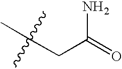 | 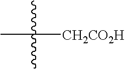 | H | H | CH₃ |
| TIV61 | 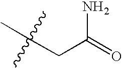 | 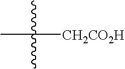 | H | H | CH₃ |
| TIV62 | 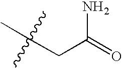 | 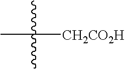 | H | H | CH₃ |
| TIV63 | 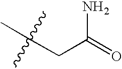 | 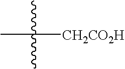 | H | H | 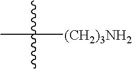 |
| TIV64 | 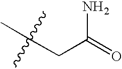 | 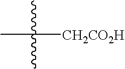 | H | H | 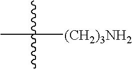 |
| TIV65 | 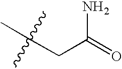 | 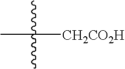 | H | H | 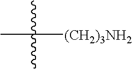 |
| TIV66 | 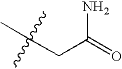 | 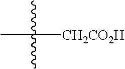 | H | H | 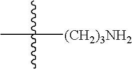 |
| TIV67 | 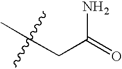 | 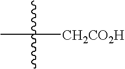 | H | H | 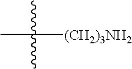 |
| TIV68 | 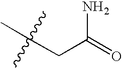 | 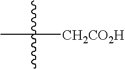 | H | H | 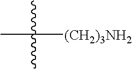 |
| TIV69 | 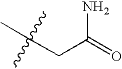 | 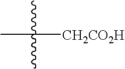 | H | H | 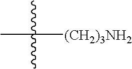 |
| TIV70 | 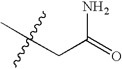 | 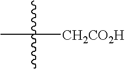 | H | H | 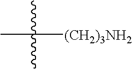 |
| TIV71 | 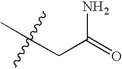 | 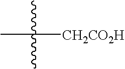 | H | H | 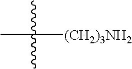 |
| TIV72 | 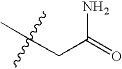 | 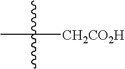 | H | H | 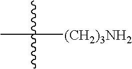 |
| TIV73 | 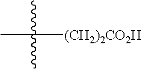 | 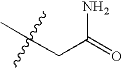 | H | H | 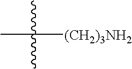 |
| TIV74 | 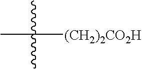 | 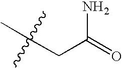 | H | H | 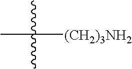 |
| TIV75 | 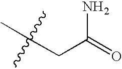 | 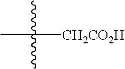 | CH₃ | H | CH₃ |
| TIV76 | 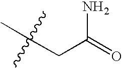 | 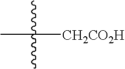 | CH₃ | H | CH₃ |
| TIV77 | 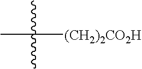 | 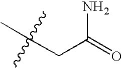 | CH₃ | H | CH₃ |
| TIV78 | 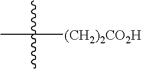 | 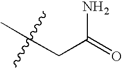 | CH₃ | H | CH₃ |

| # | R⁸ | R⁹ | R¹¹ | R¹² | R¹³ |
|---|---|---|---|---|---|
| TIV1 | 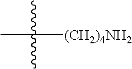 | 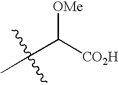 | 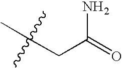 | H | 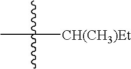 |
| TIV2 | 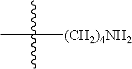 | 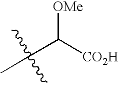 | 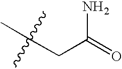 | H | 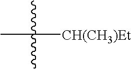 |
| TIV3 | 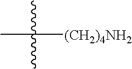 | 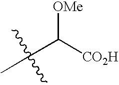 | 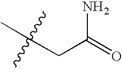 | H | 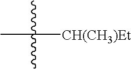 |
| TIV4 | 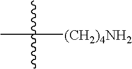 | 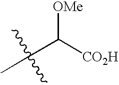 | 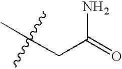 | H | 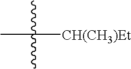 |
| TIV5 | 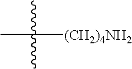 | 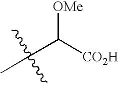 | 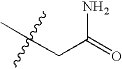 | H | 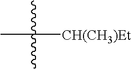 |
| TIV6 | 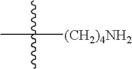 | 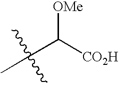 | 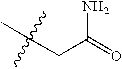 | H | 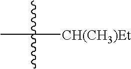 |
| TIV7 | 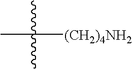 | 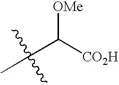 | 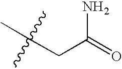 | H | 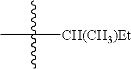 |
| TIV8 | 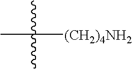 | 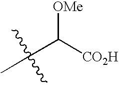 | 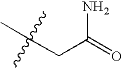 | H | 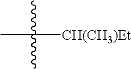 |
| TIV9 | 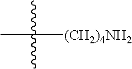 | 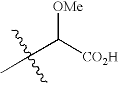 | 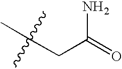 | H | 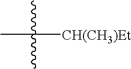 |
| TIV10 | 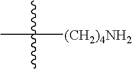 | 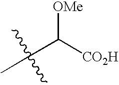 | 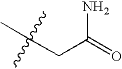 | H | 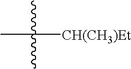 |
| TIV11 | 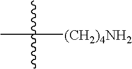 | 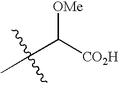 | 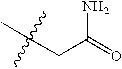 | H | 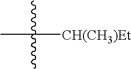 |
| TIV12 | 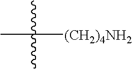 | 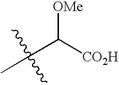 | 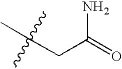 | H | 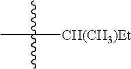 |
| TIV13 | 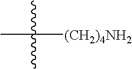 | 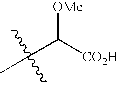 | 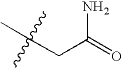 | H | 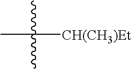 |
| TIV14 | 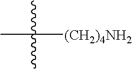 | 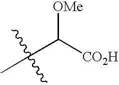 | 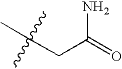 | H | 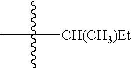 |
| TIV15 | 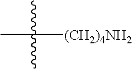 | 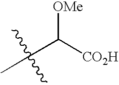 | 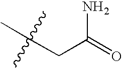 | H | 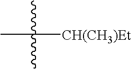 |
| TIV16 | 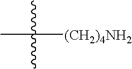 | 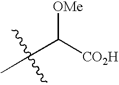 | 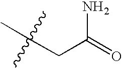 | H | 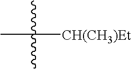 |
| TIV17 | 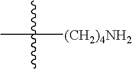 | 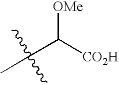 | 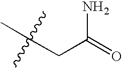 | H | 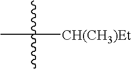 |
| TIV18 | 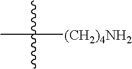 | 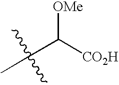 | 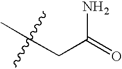 | H | 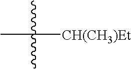 |
| TIV19 | 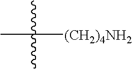 | 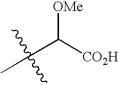 | 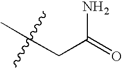 | H | 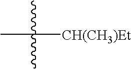 |
| TIV20 | 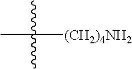 | 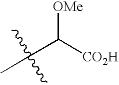 | 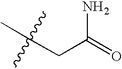 | H | 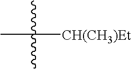 |
| TIV21 | 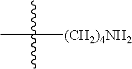 | 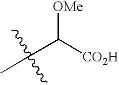 | 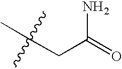 | H | 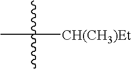 |
| TIV22 | 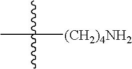 | 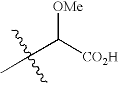 | 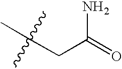 | H | 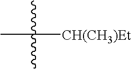 |
| TIV23 | 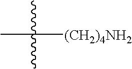 | 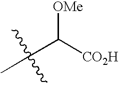 | 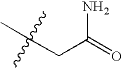 | H | 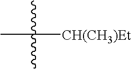 |
| TIV24 | 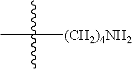 | 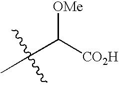 | 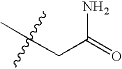 | H | 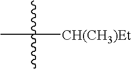 |
| TIV25 | 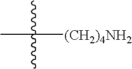 | 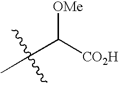 | 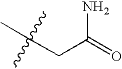 | H | 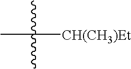 |
| TIV26 | 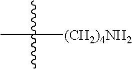 | 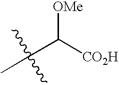 | 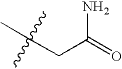 | H | 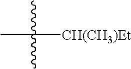 |
| TIV27 | 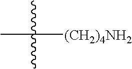 | 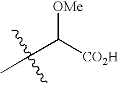 | 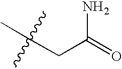 | H | 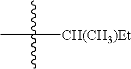 |
| TIV28 | 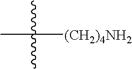 | 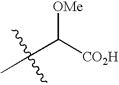 | 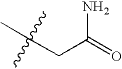 | H | 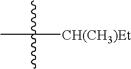 |
| TIV29 | 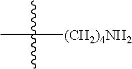 | 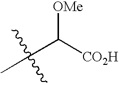 | 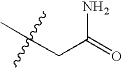 | H | 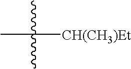 |
| TIV30 | 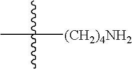 | 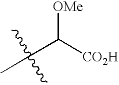 | 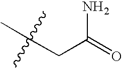 | H | 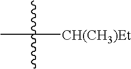 |
| TIV31 | 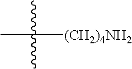 | 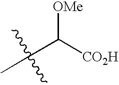 | 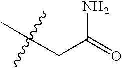 | H | 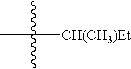 |
| TIV32 | 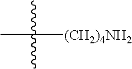 | 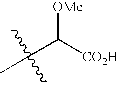 | 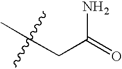 | H | 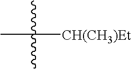 |
| TIV33 | 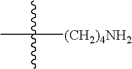 | 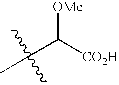 | 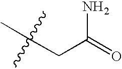 | H | 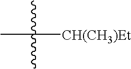 |
| TIV34 | 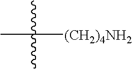 | 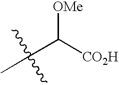 | 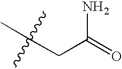 | H | 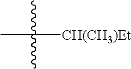 |
| TIV35 | 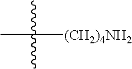 | 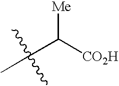 | 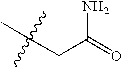 | H | 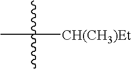 |
| TIV36 | 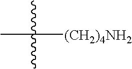 | 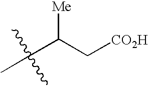 | 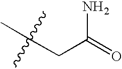 | H | 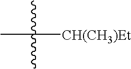 |
| TIV37 | 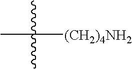 | 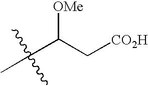 | 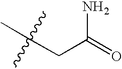 | H | 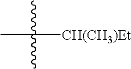 |
| TIV38 | 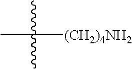 | 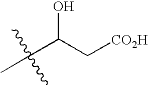 | 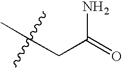 | H | 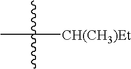 |
| TIV39 | CH₃ | 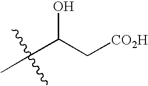 | 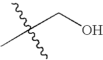 | CH₃ | 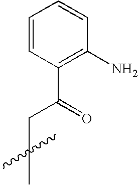 |
| TIV40 | CH₃ | 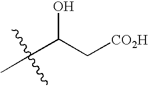 | 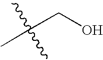 | H | 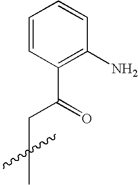 |
| TIV41 | CH₃ | 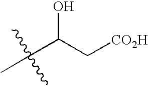 | 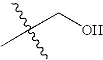 | CH₃ | 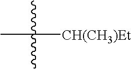 |
| TIV42 | CH₃ | 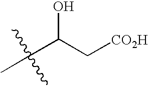 | 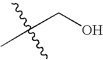 | H | 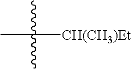 |
| TIV43 | CH₃ | 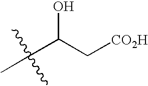 | 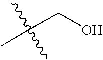 | CH₃ | 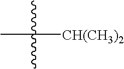 |
| TIV44 | CH₃ | 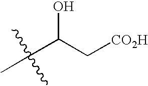 | 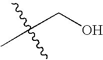 | H | 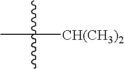 |
| TIV45 | CH₃ | 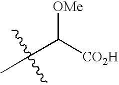 | 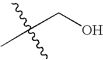 | CH₃ | 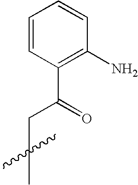 |
| TIV46 | CH₃ | 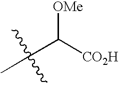 | 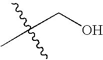 | H | 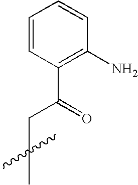 |
| TIV47 | CH₃ | 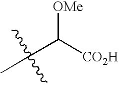 | 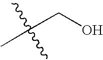 | CH₃ | 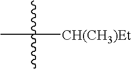 |
| TIV48 | CH₃ | 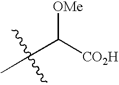 | 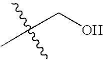 | H | 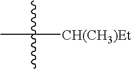 |
| TIV49 | CH₃ | 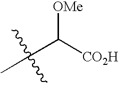 | 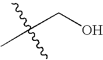 | CH₃ | 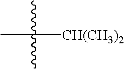 |
| TIV50 | CH₃ | 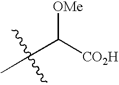 | 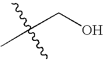 | H | 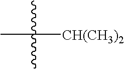 |
| TIV51 | 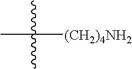 | 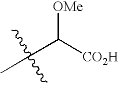 | 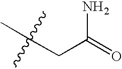 | CH₃ | 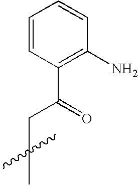 |
| TIV52 | 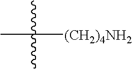 | 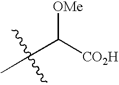 | 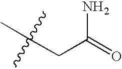 | H | 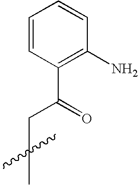 |
| TIV53 | 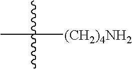 | 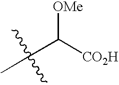 | 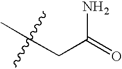 | CH₃ | 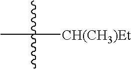 |
| TIV54 | 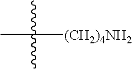 | 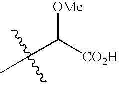 | 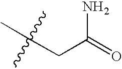 | H | 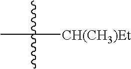 |
| TIV55 | 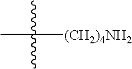 | 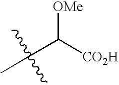 | 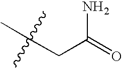 | CH₃ | 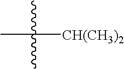 |
| TIV56 | 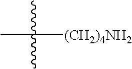 | 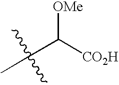 | 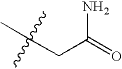 | H | 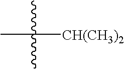 |
| TIV57 | 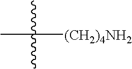 | 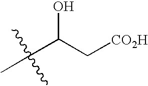 | 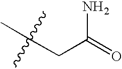 | CH₃ | 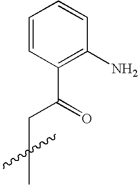 |
| TIV58 | 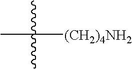 | 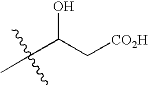 | 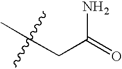 | H | 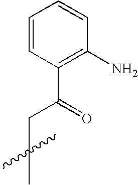 |
| TIV59 | 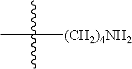 | 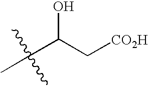 | 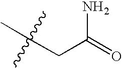 | CH₃ | 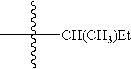 |
| TIV60 | 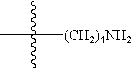 | 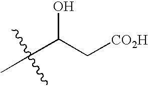 | 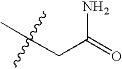 | H | 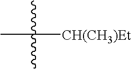 |
| TIV61 | 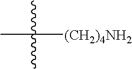 | 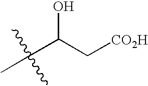 | 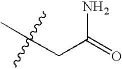 | CH₃ | 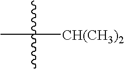 |
| TIV62 | 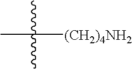 | 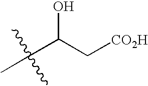 | 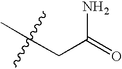 | H | 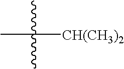 |
| TIV63 | CH₃ | 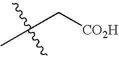 | 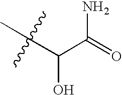 | H | 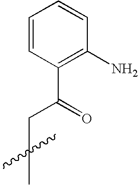 |
| TIV64 | CH₃ | 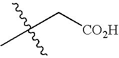 | 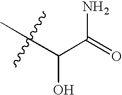 | CH₃ | 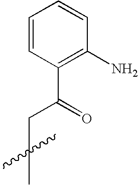 |
| TIV65 | 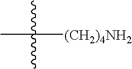 | 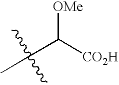 | 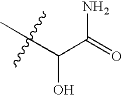 | H | 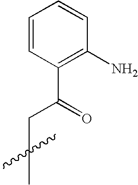 |
| TIV66 | 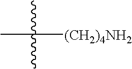 | 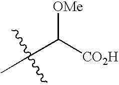 | 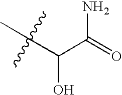 | CH₃ | 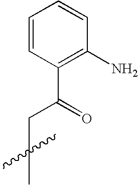 |
| TIV67 | 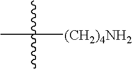 | 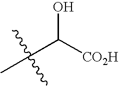 | 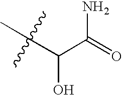 | H | 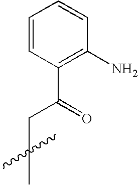 |
| TIV68 | 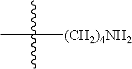 | 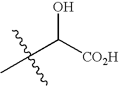 | 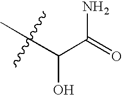 | CH₃ | 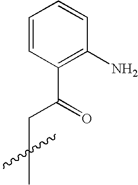 |
| TIV69 | 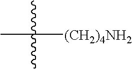 | 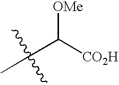 | 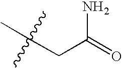 | H | 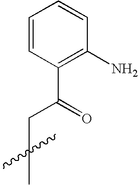 |
| TIV70 | 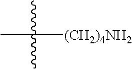 | 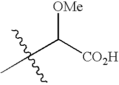 | 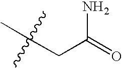 | CH₃ | 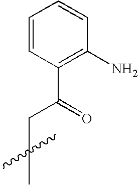 |
| TIV71 | 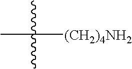 | 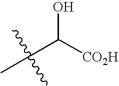 | 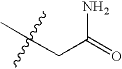 | H | 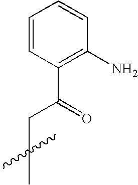 |
| TIV72 | 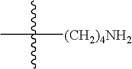 | 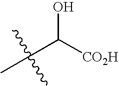 | 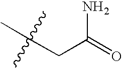 | CH₃ | 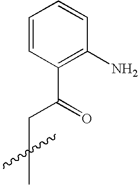 |
| TIV73 | CH₃ | 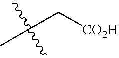 | 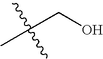 | CH₃ | 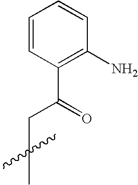 |
| TIV74 | CH₃ | 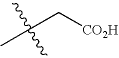 | 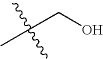 | H | 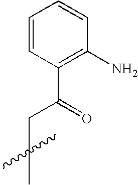 |
| TIV75 | 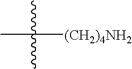 | 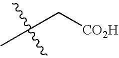 | 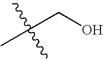 | H | 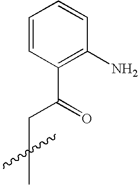 |
| TIV76 | 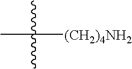 | 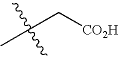 | 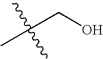 | CH₃ | 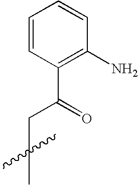 |
| TIV77 | 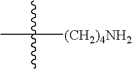 | 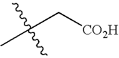 | 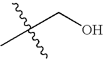 | H | 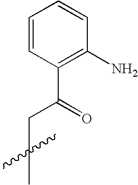 |
| TIV78 | 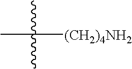 | 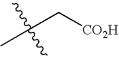 | 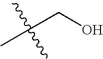 | CH₃ | 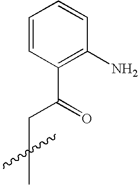 |

In another embodiment, the invention provides a compound of the Formula F1: 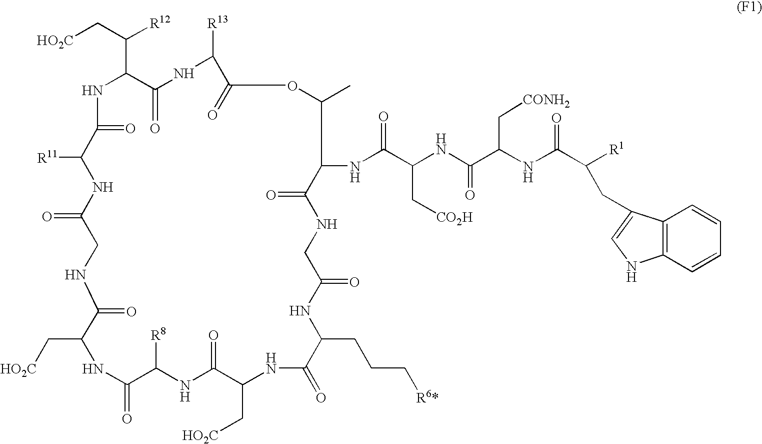
and salts thereof; wherein:
a) R⁸ is hydrogen, 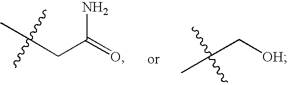
b) R¹¹ is methyl, 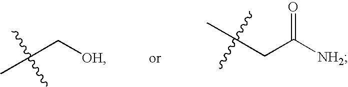
c) R¹² is H or CH₃;
d) R¹³ is CH(CH₃)₂, CH(CH₂CH₃)CH₃, 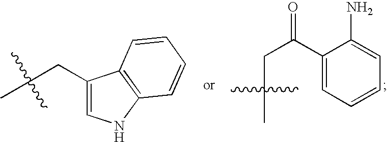
   and
e) each of R¹ and R⁶* is independently amino, monosubstituted amino, disubstituted amino, NH-amino protecting group, acylamino, ureido, guanidino, carbamoyl, sulfonamino, thioacylamino, thioureido, iminoamino, or phosphonamino.

In one embodiment of the invention, substituent R¹³ of Formula F1 is CH(CH₂CH₃)CH₃,

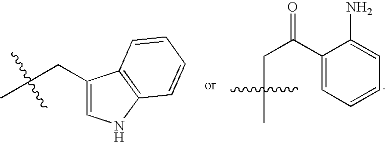

In another embodiment of the invention, a compound of Formula F1 is selected from

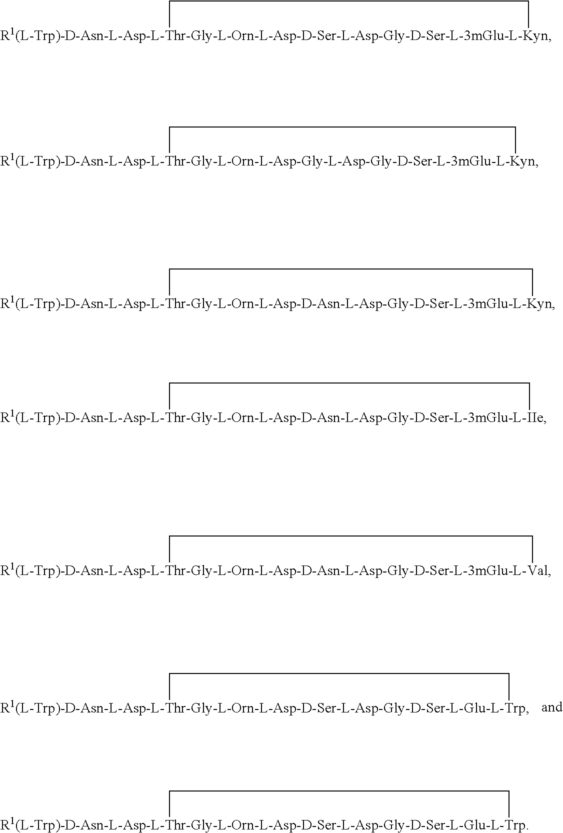

In one embodiment of the invention, substituent R¹ of Formula F1 is not C₁₀-alkanoyl when substitutent R⁸**is hydrogen or

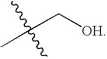

Exemplary compounds Formula F1 include, without limitation, compounds C22, C189, C201, C210, C37 and C39 (vide supra).

In another embodiment, the invention provides a compound of the Formula F2: 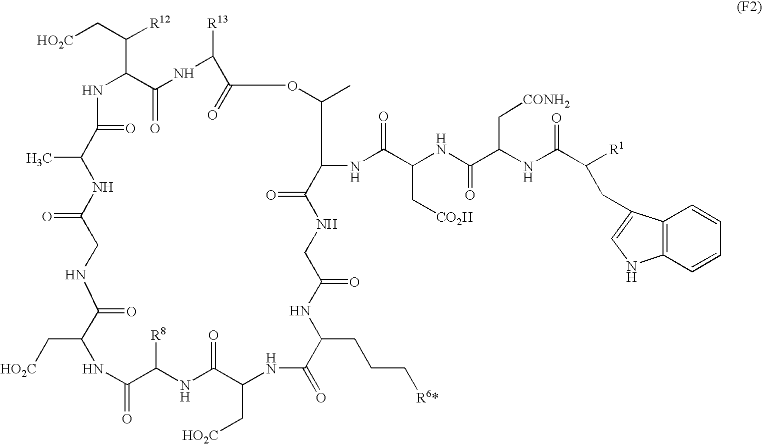
and salts thereof; wherein:
a) R⁸ is hydrogen, methyl, 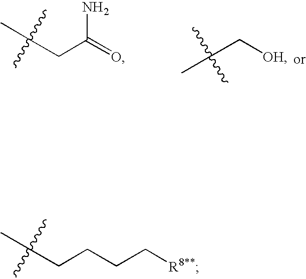
b) R² is H or CH₃;
c) R³ is CH(CH₃)₂, CH(CH₂CH₃)CH₃, 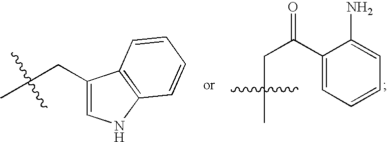
   and
d) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F2 is selected from 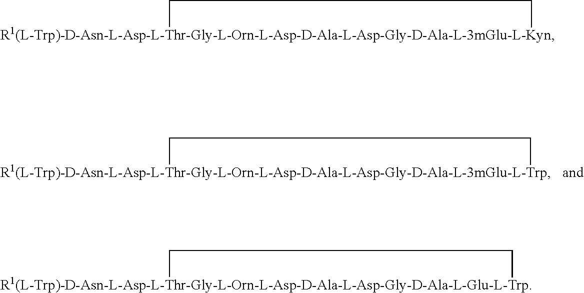
and

Exemplary compounds Formula F2 include, without limitation, compounds C46, C49, and C61 (vide supra).

In another embodiment, the invention provides a compound of the Formula F3: 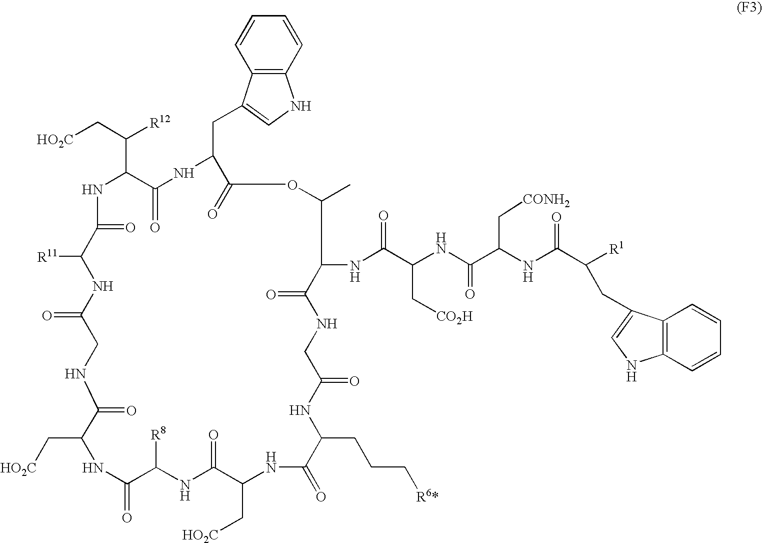
and salts thereof; wherein:
a) R⁸ is hydrogen, 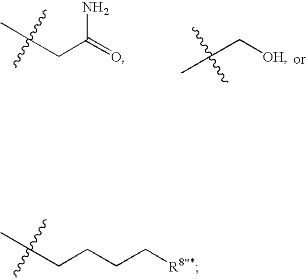
b) R¹¹ is methyl, 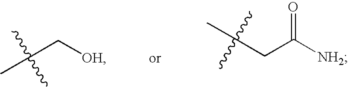
c) R¹² is H or CH₃; and
d) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

The present invention provides, in another aspect, compounds of Formula F4: 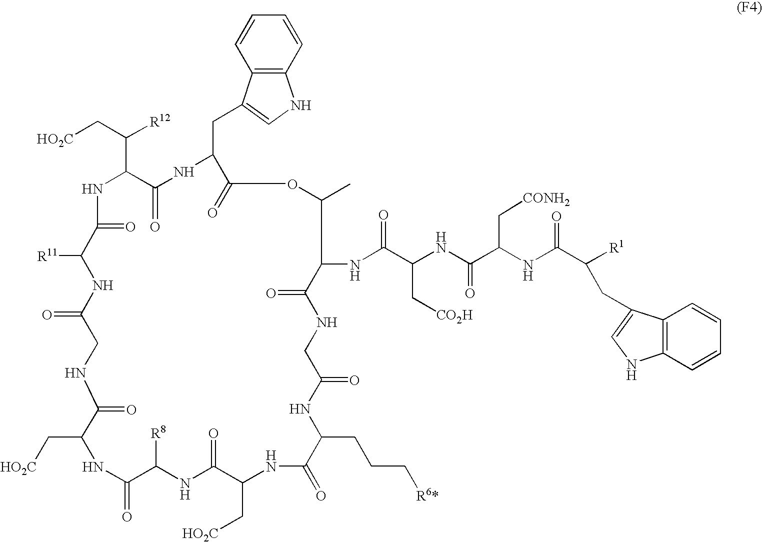
and salts thereof; wherein:
a) R⁸ is hydrogen, methyl, 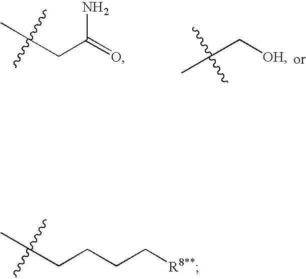
b) R¹¹ is methyl, or 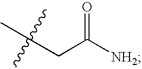
c) R¹² is H or CH₃; and
d) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment, the invention provides a compound of the Formula F5: 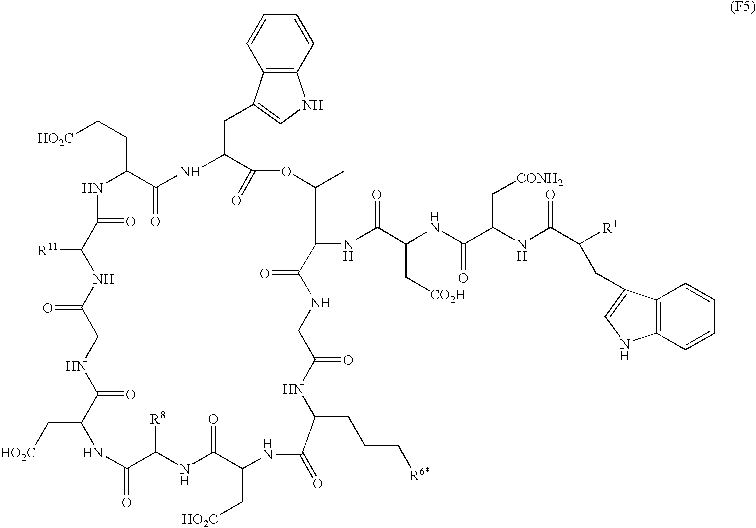
and salts thereof; wherein:
a) R⁸ is hydrogen, methyl, 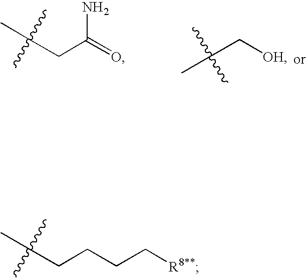
b) R¹¹ is methyl, 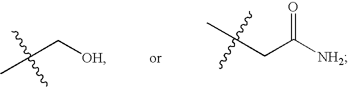
   and
c) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment, the invention provides a compound of the Formula F6: 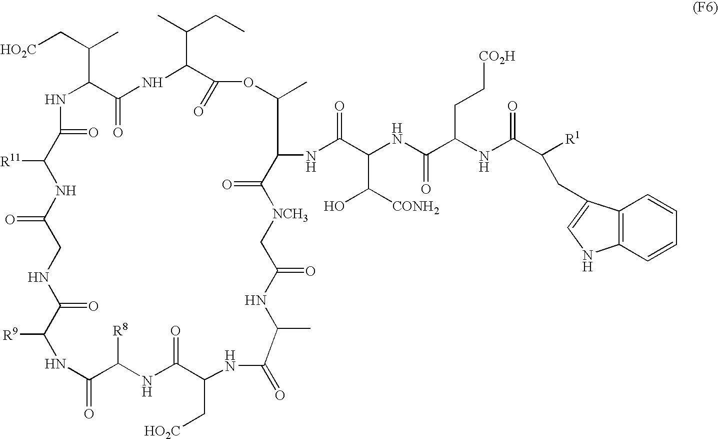
and salts thereof; wherein:
a) R⁸ is 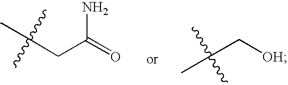
b) R⁹ is 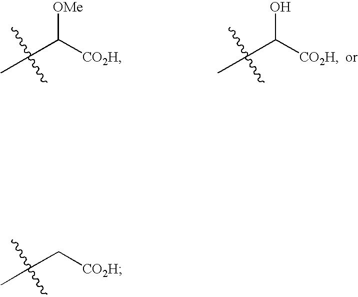
c) R¹¹ is, methyl, 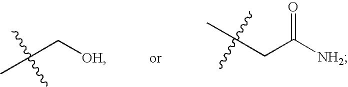
d) R¹² is H or CH₃; and
e) R¹ is amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F6 is selected from

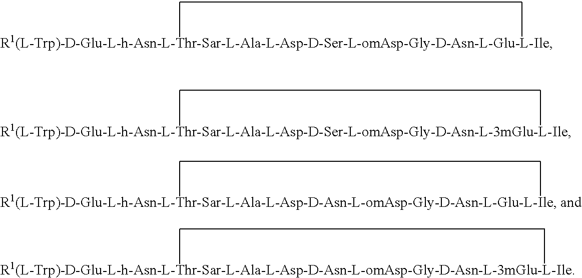

Exemplary compounds Formula F6 include, without limitation, compounds C292, C289, C307 and C304 (vide supra).

In another embodiment, the invention provides a compound of the Formula F7: 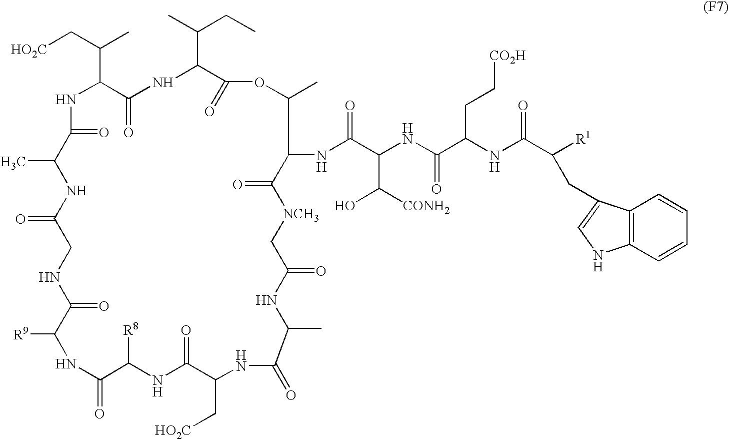
and salts thereof; wherein:
a) R⁸ is methyl, 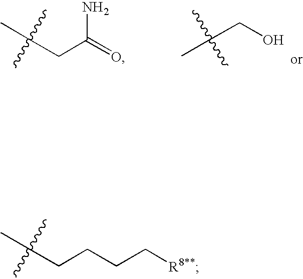
b) R⁹ is 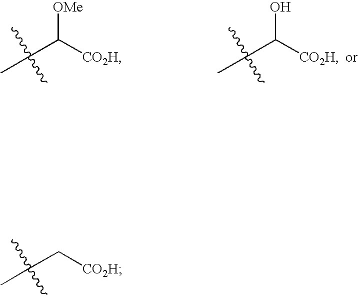
c) R¹² is H or CH₃; and
d) each of R¹ and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F7 is selected from

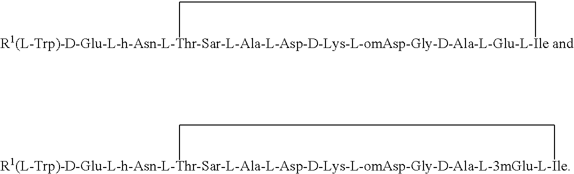

Exemplary compounds Formula F7 include, without limitation, compounds C337, and C328 (vide supra).

In another embodiment, the invention provides a compound of the Formula F8: 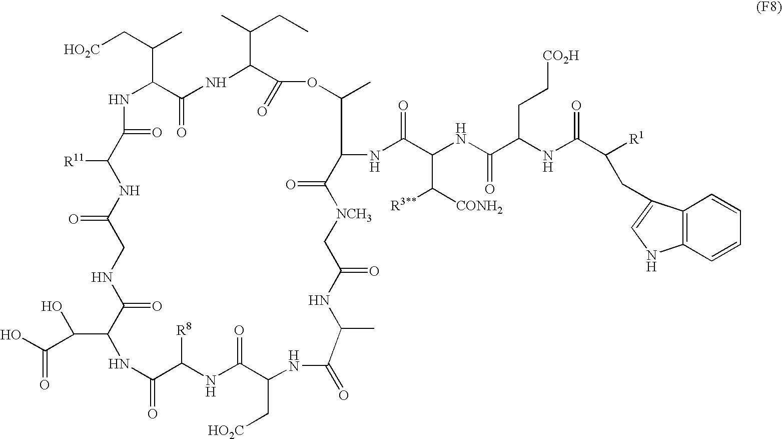
and salts thereof; wherein:
a) R³** is hydroxyl or hydrogen
b) R⁸ is methyl, 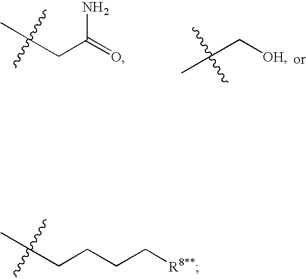
c) R¹¹ is an amino acid side chain, methyl, 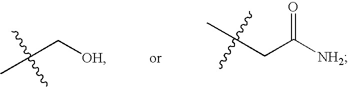
d) R¹² is H or CH₃; and
e) each of R¹ and R⁸**is independently amino, monosubstituted amino, disubstituted amino, NH-amino protecting group, acylamino, ureido, guanidino, carbamoyl, sulfonamino, thioacylamino, thioureido, iminoamino, or phosphonamino.

In one embodiment of the invention group R³** of Formula F8 is hydroxyl. This gives a compound of Formula F8A: 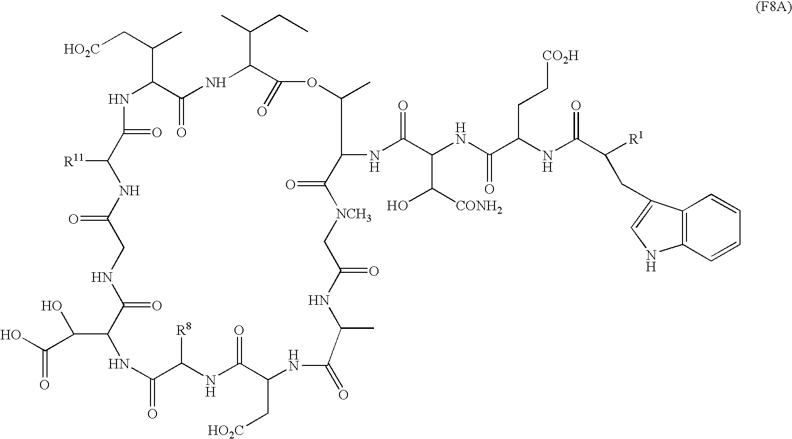
wherein R¹, R⁸, R⁸**, R¹¹, and R¹², are as described for Formula F8.

In another embodiment of the invention, a compound of Formula F8A is selected from,

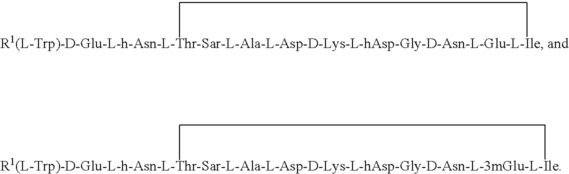

Exemplary compounds Formula F8A include, without limitation, compounds C87 and C111 (vide supra).

In another embodiment of the invention group R³** of Formula F8 is hydrogen. This gives a compound of Formula F8B: 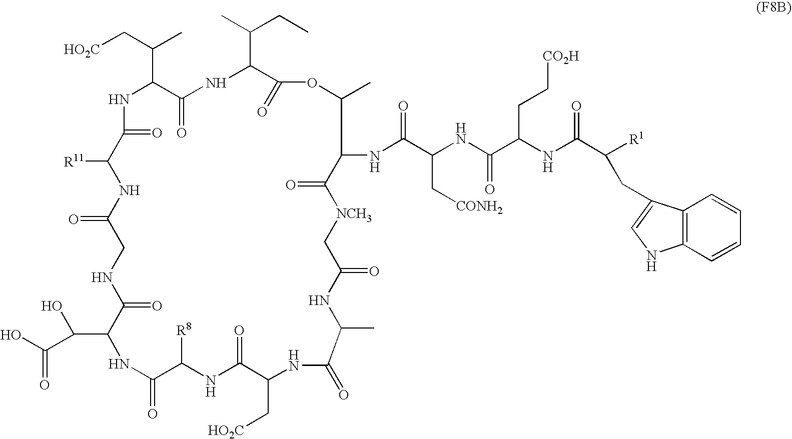
wherein R¹, R⁸, R⁸**, R¹¹, and R¹², are as described for Formula F8.

In another embodiment of the invention, a compound of Formula F8B is selected from

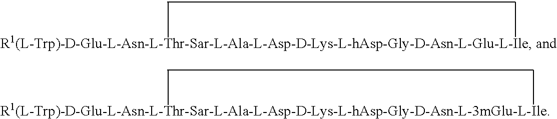

Exemplary compounds Formula F8B include, without limitation, compounds C102, and C99 (vide supra).

In another embodiment, the invention provides a compound of the Formula F9: 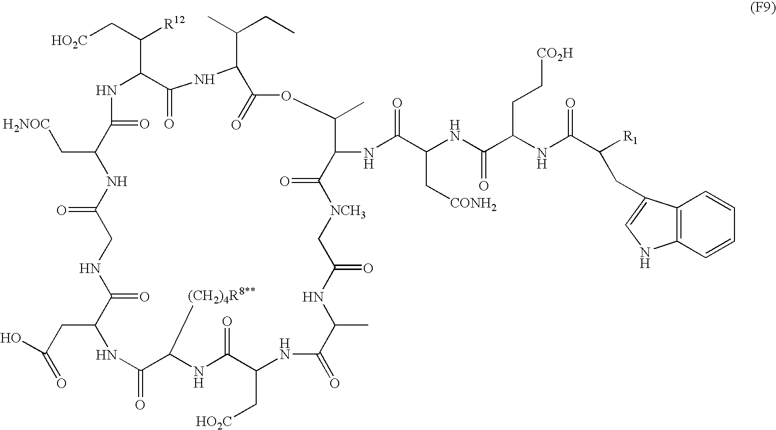
and salts thereof; wherein:
a) R¹² is H or CH₃; and
b) each of R¹, and R⁸** is independently amino, monosubstituted amino, disubstituted amino, NH-amino protecting group, acylamino, ureido, guanidino, carbarnoyl, sulfonamino, thioacylamino, thioureido, iminoamino, or phosphonamino.

In one embodiment of the invention, substituent group R¹² of Formula F9 is methyl.

In another embodiment of the invention, a compound of Formula F9 is selected from

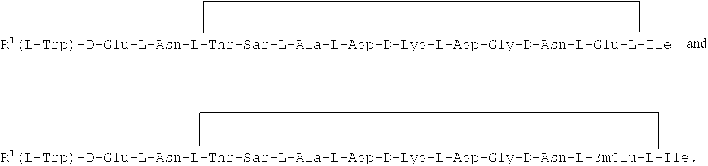

Exemplary compounds Formula F2 include, without limitation, compounds C105, and C108 (vide supra).

In another embodiment, the invention provides a compound of the Formula F10: 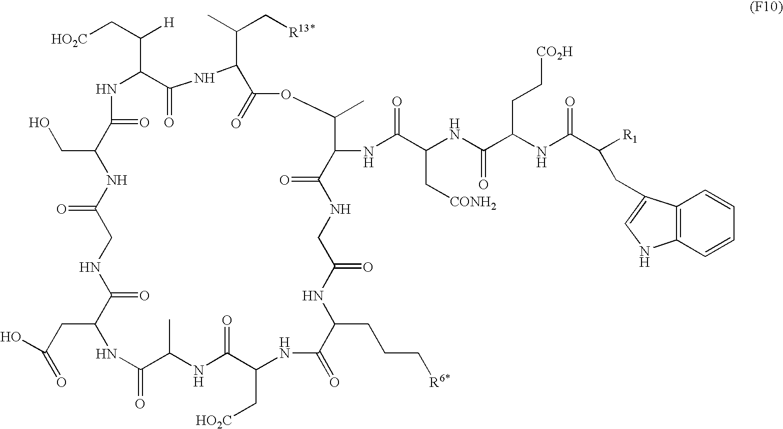
and salts thereof; wherein:
a) R¹³* is H or CH₃; and
b) each of R¹, and R⁶* is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F10 is selected from

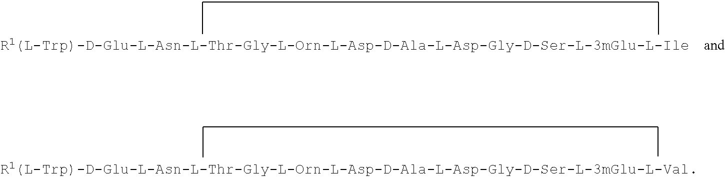

Exemplary compounds Formula F10 include, without limitation, compounds C259, and C262 (vide supra).

In another embodiment, the invention provides a compound of the Formula F11: 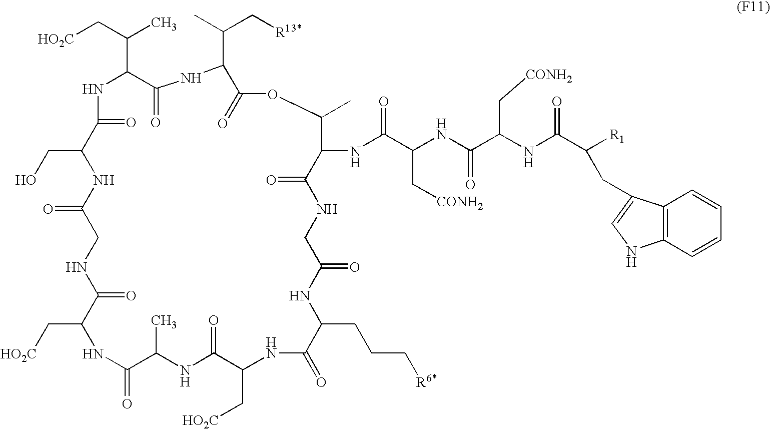
and salts thereof; wherein:
a) R¹³* is H or CH₃; and
b) each of R¹, and R⁶* is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F11 is selected from

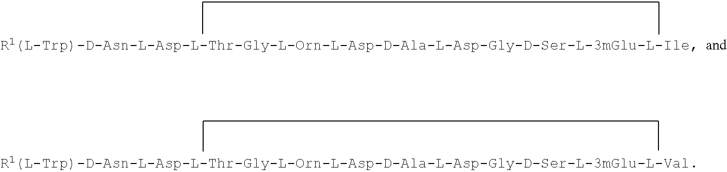

Exemplary compounds Formula F11 include, without limitation, compounds C4, and C8 (vide supra).

In another embodiment, the invention provides a compound of the Formula F12: 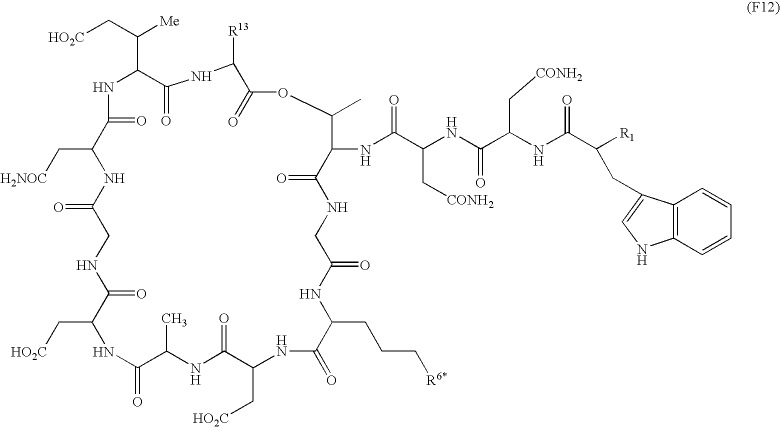
and salts thereof; wherein:
a) R¹³ is CH(CH₂CH₃)CH₃ or 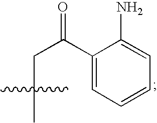
   and
b) each of R¹ and R⁶* is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F12 is selected from

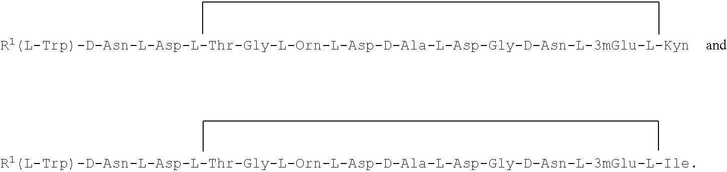

Exemplary compounds Formula F12 include, without limitation, compounds C233, and C221 (vide supra).

In another embodiment, the invention provides a compound of the Formula F13: 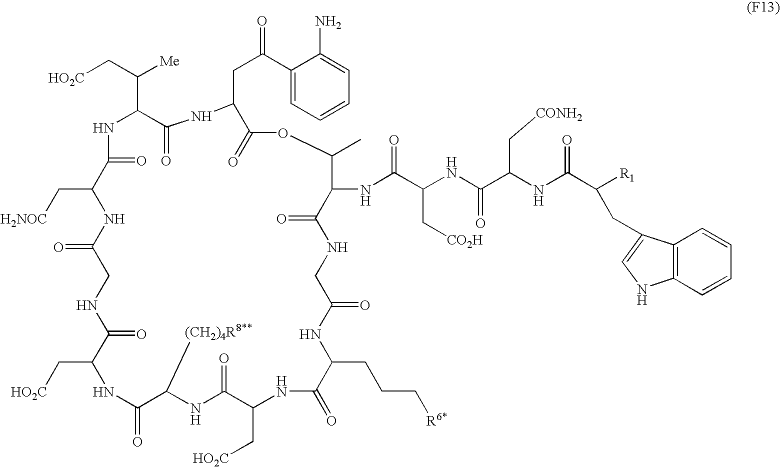
and salts thereof; wherein each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F13 is selected from

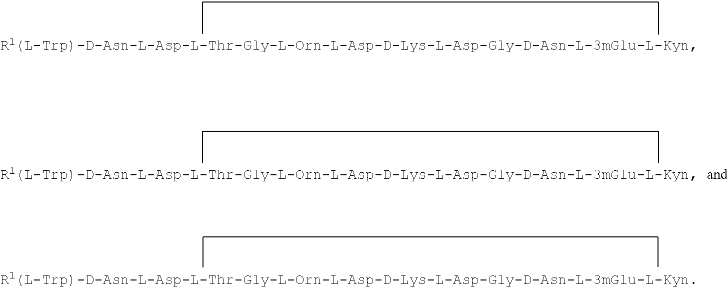

Exemplary compounds Formula F13 include, without limitation, compounds C236, C237, and C238 (vide supra).

In another embodiment, the invention provides a compound of the Formula F14: 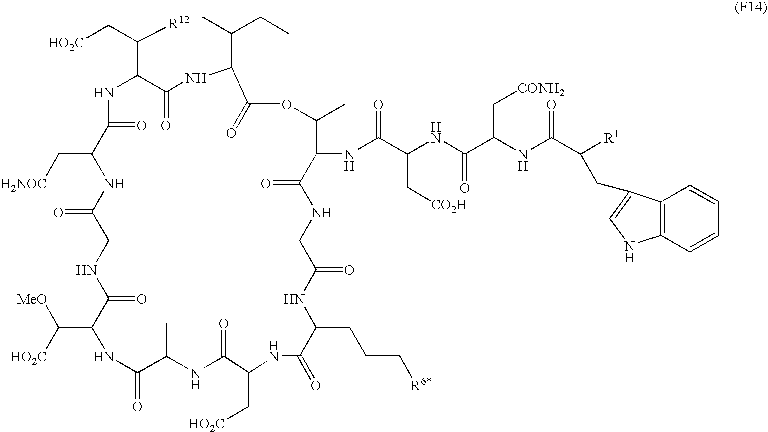
and salts thereof; wherein:
a) R¹² is H or CH₃; and
b) each of R¹ and R⁶* is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F14 is selected from

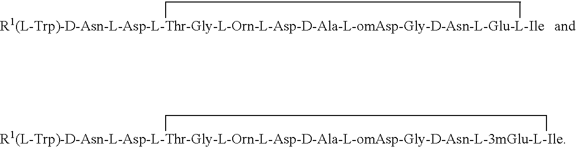

Exemplary compounds Formula F14 include, without limitation, compounds C283, and C277 (vide supra).

In another embodiment, the invention provides a compound of the Formula F15: 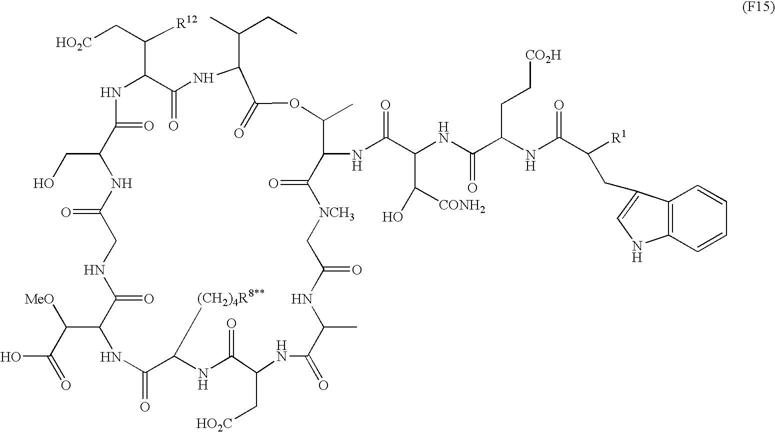
and salts thereof; wherein:
a) R¹² is H or CH₃; and
b) each of R¹ and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In one embodiment of the invention, substituent group R¹² of Formula F15 is methyl.

In another embodiment of the invention, a compound of Formula F15 is selected from

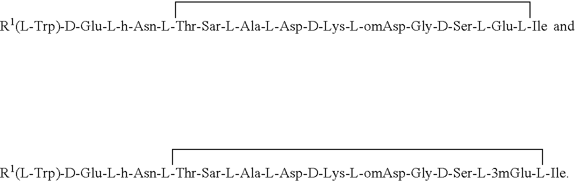

Exemplary compounds Formula F15 include, without limitation, compounds C325, and C153 (vide supra).

In another embodiment, the invention provides a compound of the Formula F16: 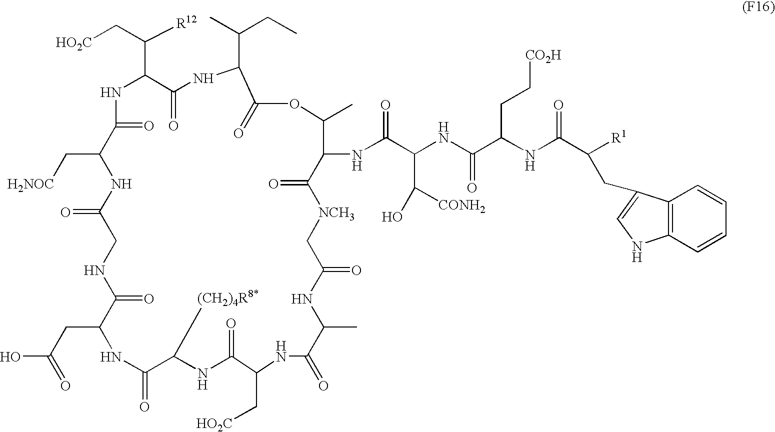
and salts thereof; wherein:
a) R¹² is H or CH₃, and
b) each of R¹ and R⁸* is independently amino, monosubstituted amino, disubstituted amino, NH-amino protecting group, acylamino, ureido, guanidino, carbamoyl, sulfonamino, thioacylamino, thioureido, iminoamino, or phosphonamino.

In one embodiment of the invention, substituent group R¹² of Formula F16 is methyl.

In another embodiment of the invention, a compound of Formula F16 is selected from

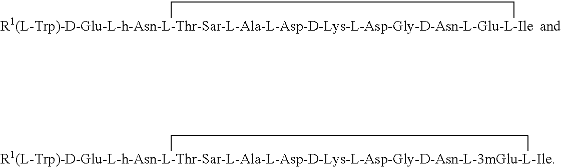

Exemplary compounds Formula F16 include, without limitation, compounds C90, and C114 (vide supra).

In another embodiment, the invention provides a compound of the Formula F17: 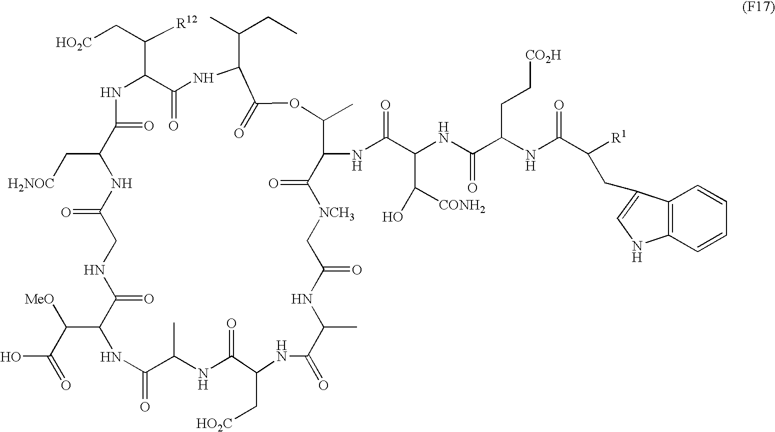
and salts thereof; wherein:
a) R¹² is H or CH₃; and
b) R¹ is amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F17 is selected from

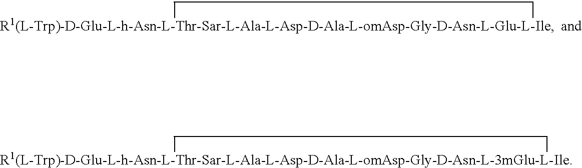

Exemplary compounds Formula F17 include, without limitation, compounds C316, and C319 (vide supra).

In another embodiment, the invention provides a compound of the Formula F18: 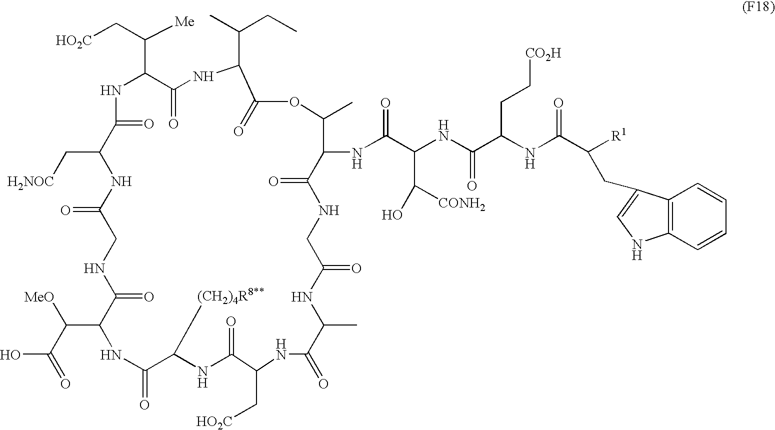
and salts thereof;
wherein each of R¹ and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F18 is

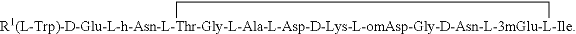

An exemplary compound of Formula F18 is, without limitation, compound C180 (vide supra).

In another embodiment, the invention provides a compound of the Formula F19: 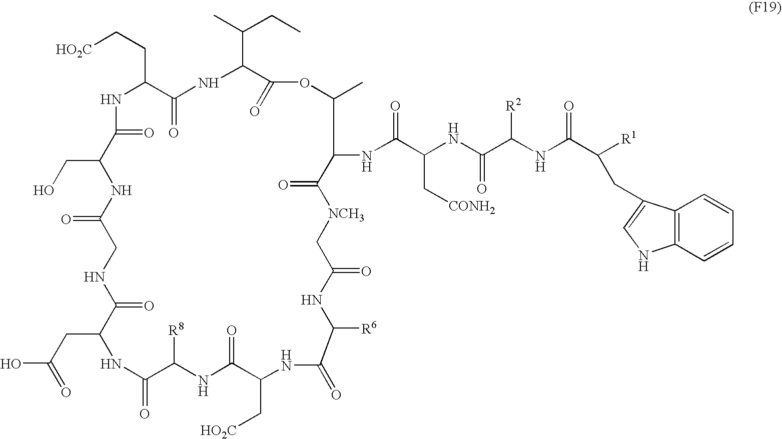
and salts thereof; wherein:
a) R² is 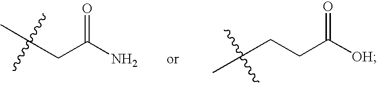
b) R⁶ is methyl or 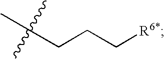
c) R⁸ is methyl or 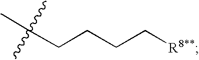
   and
d) each of R¹, R⁶*, and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F19 is selected from

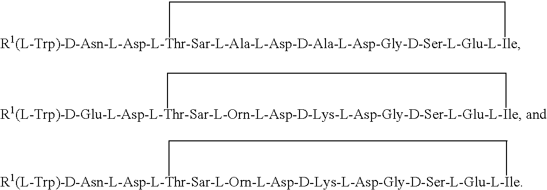

Exemplary compounds Formula F19 include, without limitation, compounds C86, C359, and C356 (vide supra).

In another embodiment, the invention provides a compound of the Formula F20: 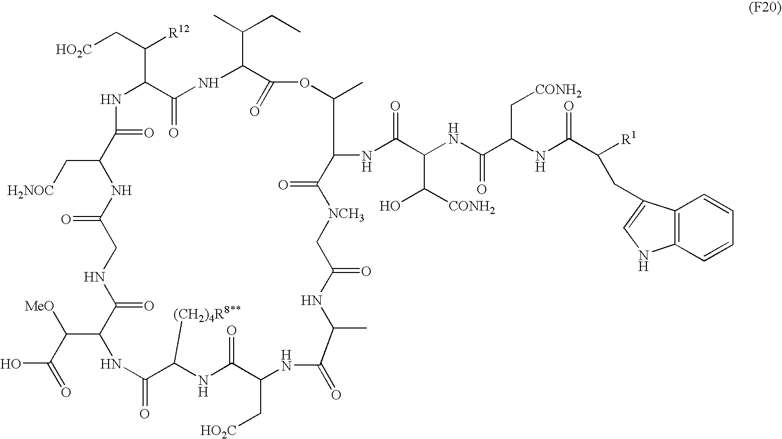
and salts thereof; wherein:
a) R¹² is H or CH₃; and
b) each of R¹ and R⁸** is amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F20 is selected from

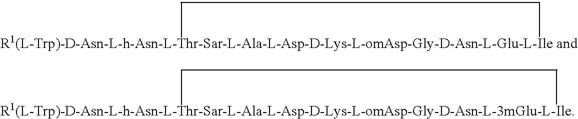

Exemplary compounds Formula F20 include, without limitation, compounds C343, and C340 (vide supra).

In another embodiment, the invention provides a compound of the Formula F21 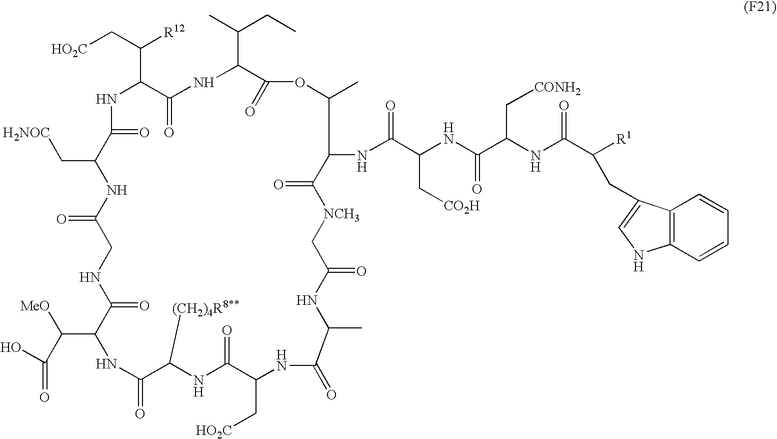
and salts thereof; wherein:
a) R¹ is 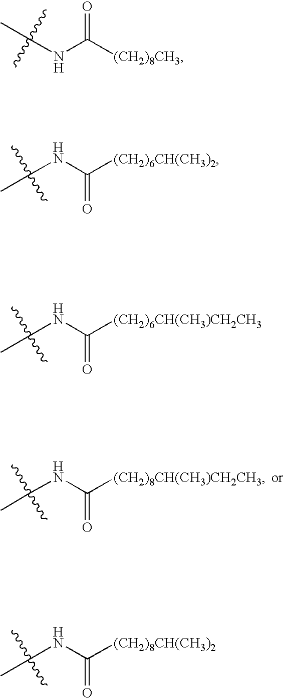
b) R¹² is H or CH₃, and
c) R⁸** is amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F21 is selected from

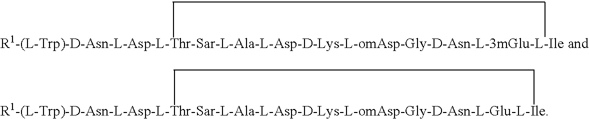

Exemplary compounds Formula F21 include, without limitation, compounds C265, and C271 (vide supra).

In another embodiment, the invention provides a compound of the Formula F22 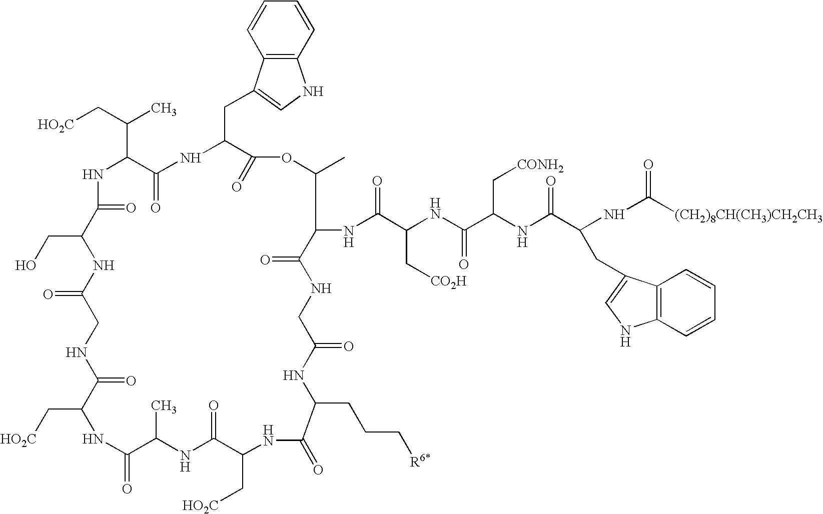
(F22)
and salts thereof, wherein:
R⁶* is amino, monosubstituted amino, disubstituted amino, NH-amino protecting group, acylamino, ureido, guanidino, carbamoyl, sulfonamino, thioacylamino, thioureido, iminoamino, or phosphonamino.

In another embodiment of the invention, a compound of Formula F22 is

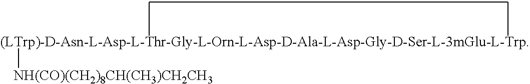

An exemplary compound Formula F22 includes, without limitation, compound C3 (vide supra).

In one embodiment of the invention, substituent R¹ of any of the compounds of Formula F1-F20 is amino, acylamino, NH-amino protecting group or carbamoyl. In another embodiment of the invention, substituent R¹ of any of the compounds of Formula F1-F20 is a C₁₀-C₁₃ alkanoylamino. In yet another embodiment of the invention, substituent R¹ of any of the compounds of Formula F1-F20 is 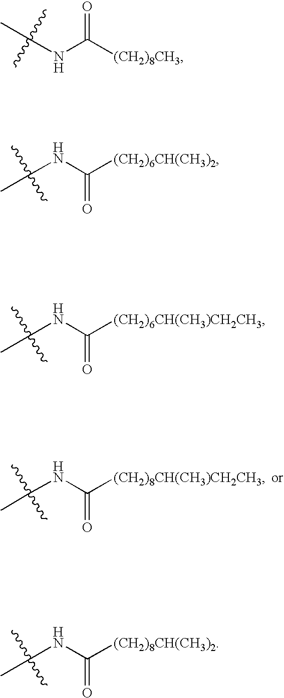
In yet another embodiment of the invention, substituent R¹ of any of the compounds of Formula F1-F20 is

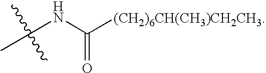

In one embodiment of the invention, substituent R⁶* of any of the compounds of Formula F1-F5, F10-F14, F19 and F22 is amino, NH-amino protecting group or carbamoyl. In another embodiment of the invention, substituent R⁶* of any of the compounds of Formula of F1-F5, F10-F14, F19 and F22 is amino.

In one embodiment of the invention, substituent R⁸** of any of the compounds of Formula F2-F5, F7-F9, F13, F15, F16, F18 and F20-F21 is amino, NH-amino protecting group or carbamoyl. In another embodiment of the invention, substituent R⁸** of any of the compounds of Formula F2-F5, F7-F9, F13, F15, F16, F18 and F20-F21 is amino. It will be understood by one of skill in the art that the compounds of the invention, particularly compounds of Formula I and Formula F1-F22, are useful as intermediates for the preparation of other compounds of Formula I and Formula F1-F22. Particularly useful compounds that are also intermediates are compounds of Formula I, F2-F5, F13 and F19 wherein at least one of R¹, R⁶ or R⁸**is amino, NH-amino protecting group or carbamoyl; compounds of Formula F1 or F10-F14 wherein at least one of R¹ or R⁶* is amino, NH-amino protecting group or carbamoyl; compounds of Formula F7-9, F15-16, F18 and F20 wherein at least one of R¹ or R⁸**is amino, NH-amino protecting group or carbamoyl; compounds of Formula F22 wherein R⁶* is amino, NH-amino protecting group or carbamoyl; compounds of Formula F21 wherein R⁸** is amino, NH-amino protecting group or carbamoyl; and compounds of Formula F6 and F17 wherein R¹ is amino, NH-amino protecting group or carbamoyl.

Pharmaceutical Compositions and Methods of Use Thereof

The instant invention provides pharmaceutical compositions or formulations comprising, in one embodiment, compounds of Formula I or compounds of any of Formula F1-F22, or salts thereof.

Compounds of the present invention, preferably compounds Formula I or compounds of any of Formula F1-F22, or pharmaceutically acceptable salts thereof, can be formulated for oral, intravenous, intramuscular, subcutaneous or parenteral administration for the therapeutic or prophylactic treatment of diseases, particularly bacterial infections. For oral or parenteral administration, compounds of the present invention can be mixed with conventional pharmaceutical carriers and excipients and used in the form of tablets, capsules, elixirs, suspensions, syrups, wafers and the like. The compositions comprising a compound of this invention will contain from about 0.1 to about 99% by weight of the active compound, and more generally from about 10 to about 30%.

The pharmaceutical preparations disclosed herein are prepared in accordance with standard procedures and are administered at dosages that are selected to reduce, prevent or eliminate the infection (See, e.g., “Remington's Pharmaceutical Sciences”, Mack Publishing Company, Easton, Pa. and “Goodman and Gilman's The Pharmaceutical Basis of Therapeutics”, Pergamon Press, New York, N.Y., the contents of which are incorporated herein by reference, for a general description of the methods for administering various antimicrobial agents for human therapy). The compositions of the present invention, preferably compositions of Formulas I or compositions of any of Formulas F1-F22, can be delivered using controlled (e.g., capsules) or sustained release delivery systems (e.g., bioerodable matrices). Exemplary delayed release delivery systems for drug delivery that are suitable for administration of the compositions of the invention, preferably compositions of Formula I or any of Formulas F1-F22, are described in U.S. Pat. Nos. 4,452,775 (issued to Kent), 5,239,660 (issued to Leonard), and 3,854,480 (issued to Zaffaroni).

The pharmaceutically-acceptable compositions of the present invention comprise one or more compounds of the invention, preferably compounds of Formula I or compounds of any of Formulas F1-F22, in association with one or more nontoxic, pharmaceutically-acceptable carriers and/or diluents and/or adjuvants and/or excipients, collectively referred to herein as “carrier” materials, and if desired other active ingredients. The compositions may contain common carriers and excipients, such as corn starch or gelatin, lactose, sucrose, microcrystalline cellulose, kaolin, mannitol, dicalcium phosphate, sodium chloride and alginic acid. The compositions may contain croscarmellose sodium, microcrystalline cellulose, corn starch, sodium starch glycolate and alginic acid.

Tablet binders that can be included are acacia, methylcellulose, sodium carboxymethylcellulose, polyvinylpyrrolidone (Povidone), hydroxypropyl methylcellulose, sucrose, starch and ethylcellulose.

Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. It may also be desirable to add a coloring agent to make the dosage form more aesthetic in appearance or to help identify the product.

For oral use, solid formulations such as tablets and capsules are particularly useful. Sustained release or enterically coated preparations may also be devised. For pediatric and geriatric applications, suspensions, syrups and chewable tablets are especially suitable. For oral administration, the pharmaceutical compositions are in the form of, for example, a tablet, capsule, suspension or liquid. The pharmaceutical composition is preferably made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example, potato starch, flavoring or coloring agents, or acceptable wetting agents. Oral liquid preparations generally are in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For intravenous (IV) use, a compound of the present invention can be dissolved or suspended in any of the commonly used intravenous fluids and administered by infusion. Intravenous fluids include, without limitation, physiological saline or Ringer's solution. Intravenous administration may be accomplished by using, without limitation, syringe, minipump or intravenous line.

Formulations for parenteral administration can be in the form of aqueous or non-aqueous isotonic sterile injection solutions or suspensions. These solutions or suspensions can be prepared from sterile powders or granules having one or more of the carriers mentioned for use in the formulations for oral administration. The compounds can be dissolved in polyethylene glycol, propylene glycol, ethanol, corn oil, benzyl alcohol, sodium chloride, and/or various buffers.

For intramuscular preparations, a sterile formulation of a compound of the present invention, or a suitable soluble salt form of the compound, for example the hydrochloride salt, can be dissolved and administered in a pharmaceutical diluent such as Water-for-Injection (WFI), physiological saline or 5% glucose. A suitable insoluble form of the compound may be prepared and administered as a suspension in an aqueous base or a pharmaceutically acceptable oil base, e.g., an ester of a long chain fatty acid such as ethyl oleate.

A dose of an intravenous, intramuscular or parental formulation of a compound of the present invention may be adminstered as a bolus or by slow infusion. A bolus is a dose that is administered in less than 30 minutes. In a preferred embodiment, a bolus is administered in less than 15 or less than 10 minutes. In a more preferred embodiment, a bolus is administered in less than 5 minutes. In an even more preferred embodiment, a bolus is administered in one minute or less. An infusion is a dose that is administered at a rate of 30 minutes or greater. In a preferred embodiment, the infusion is one hour or greater. In another embodiment, the infusion is substantially constant.

For topical use the compounds of the present invention, preferably compounds of Formula I or compounds of any of Formula F1-F22, can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient.

For application to the eyes or ears, the compounds of the present invention, preferably compounds Formula I or compounds of any of Formula F1-F22, can be presented in liquid or semi-liquid form formulated in hydrophobic or hydrophilic bases as ointments, creams, lotions, paints or powders.

For rectal administration the compounds of the present invention, preferably compounds Formula I or compounds of any of Formula F1-F22, can be administered in the form of suppositories admixed with conventional carriers such as cocoa butter, wax or other glyceride.

Alternatively, the compounds of the present invention, in one embodiment, compounds of Formula I or compounds of any of Formulas F1-F22, can be in powder form for reconstitution in the appropriate pharmaceutically acceptable carrier at the time of delivery. In another embodiment, the unit dosage form of the compound can be a solution of the compound or preferably a salt thereof in a suitable diluent in sterile, hermetically sealed ampoules or sterile syringes. The concentration of the compound in the unit dosage may vary, e.g. from about 1 percent to about 50 percent, depending on the compound used and its solubility and the dose desired by the physician. If the compositions contain dosage units, each dosage unit preferably contains from 1-500 mg of the active material. For adult human treatment, the dosage employed preferably ranges from 5 mg to 10 g, per day, depending on the route and frequency of administration.

In another aspect, the invention provides a method for inhibiting the growth of microorganisms, preferably bacteria, comprising contacting said organisms with a compound of the present invention under conditions which permit contact of the compound with said organism and with said microorganism. Such conditions are known to one skilled in the art and are exemplified in the Examples. This method involves contacting a microbial cell with a therapeutically-effective amount of compound(s) of the invention, preferably compound(s) of s Formula I or compound(s) of any of Formula F1-F22 in vivo or in vitro.

According to this aspect of the invention, the novel compositions disclosed herein are placed in a pharmaceutically acceptable carrier and are delivered to a recipient subject (preferably a human) in accordance with known methods of drug delivery. In general, the methods of the invention for delivering the compositions of the invention in vivo utilize art-recognized protocols for delivering the agent with the only substantial procedural modification being the substitution of the compounds of the present invention, preferably compounds of Formula I or compounds of any of Formula F1-F22, for the drugs in the art-recognized protocols. Likewise, the methods for using the claimed composition for treating cells in culture, for example, to eliminate or reduce the level of bacterial contamination of a cell culture, utilize art-recognized protocols for treating cell cultures with antibacterial agent(s) with the only substantial procedural modification being the substitution of the compounds of the invention, preferably compounds of Formula I or compounds of any of Formula F1-F22, for the agents used in the art-recognized protocols.

In one embodiment, the invention provides a method for treating an infection, especially those caused by gram-positive bacteria, in a subject with a therapeutically-effective amount of a compound of the invention. Exemplary procedures for delivering an antibacterial agent are described in U.S. Pat. No. 5,041,567, and PCT patent application number EP94/02552 (publication number WO 95/05384), the entire contents of which documents are incorporated in their entirety herein by reference. As used herein, the phrase “therapeutically-effective amount” means an amount of a compound of the present invention that prevents the onset, alleviates the symptoms, or stops the progression of a bacterial infection. The term “treating” is defined as administering, to a subject, a therapeutically-effective amount of a compound of the invention both to prevent the occurrence of an infection and to control or eliminate an infection. The term “subject,” as described herein, is defined as a mammal, a plant or a cell culture. In a preferred embodiment, a subject is a human or other animal patient in need of antibacterial treatment.

The method comprises administering to the subject an effective dose of a compound of the present invention. An effective dose is generally between about 0.1 and about 100 mg/kg of a compound of the invention or a pharmaceutically acceptable salt thereof. A preferred dose is from about 0.1 to about 50 mg/kg of a compound of the invention or a pharmaceutically acceptable salt thereof. A more preferred dose is from about 1 to 25 mg/kg of a compound of the invention or a pharmaceutically acceptable salt thereof. An effective dose for cell culture is usually between 0.1 and 1000 μg/mL, more preferably between 0.1 and 200 μg/mL.

Compositions containing the compounds of the invention can be administered as a single daily dose or in multiple doses per day. The treatment regime may require administration over extended periods of time, e.g., for several days or for from two to four weeks. The amount per administered dose or the total amount administered will depend on such factors as the nature and severity of the infection, the age and general health of the patient, the tolerance of the patient to the compound and the microorganism or microorganisms involved in the infection. A method of administration to a patient of daptomycin, another member of the depsipeptide compound class, is disclosed in U.S. Pat. Nos. 6,468,967 and 6,852,689, the contents of which are herein incorporated by reference.

A compound of the present invention may also be administered in the diet or feed of a patient or animal. If administered as part of a total dietary intake, the amount of compound employed can be less than 1% by weight of the diet and preferably no more than 0.5% by weight. The diet for animals can be normal foodstuffs to which the compound can be added or it can be added to a premix.

The present invention also provides methods of administering a compound of the invention, preferably a compound of Formula I or a compound of any of Formulas F1-F22, or a pharmaceutical composition thereof to a subject in need thereof in an amount that is efficacious in reducing, ameliorating or eliminating the bacterial infection. The compound may be administered orally, parenterally, by inhalation, topically, rectally, nasally, buccally, vaginally, or by an implanted reservoir, external pump or catheter. The compound may be prepared for opthalmic or aerosolized uses. The compounds of the present invention can be administered as an aerosol. A preferred aerosol delivery vehicle is an anhydrous or dry powder inhaler. Compounds of Formula I or compounds of any of Formula F1-F22, or a pharmaceutical composition thereof may also be directly injected or administered into an abscess, ventricle or joint. Parenteral administration includes subcutaneous, intravenous, intramuscular, intra-articular, intra-synovial, cisternal, intrathecal, intrahepatic, intralesional and intracranial injection or infusion. In a preferred embodiment, the compounds of the present invention are administered intravenously, subcutaneously or orally. In a preferred embodiment for administering a compound according to Formula I or a compound of any of Formula F1-F22 to a cell culture, the compound may be administered in a nutrient medium.

The method of the instant invention may be used to treat a subject having a bacterial infection in which the infection is caused or exacerbated by any type of bacteria, particularly gram-positive bacteria. In one embodiment, a compound of the present invention or a pharmaceutical composition thereof is administered to a patient according to the methods of this invention. In a preferred embodiment, the bacterial infection may be caused or exacerbated by gram-positive bacteria. These gram-positive bacteria include, but are not limited to, methicillin-susceptible and methicillin-resistant staphylococci (including Staphylococcus aureus, S. epidermidis, S. haemolyticus, S. hominis, S. saprophyticus, and coagulase-negative staphylococci), glycopeptide intermediary-susceptible S. aureus (GISA), vancomycin-resistant Staphylococcus aureus (VRSA), penicillin-susceptible and penicillin-resistant streptococci (including Streptococcus pneumoniae, S. pyogenes, S. agalactiae, S. avium, S. bovis, S. lactis, S. sangius and Streptococci Group C, Streptococci Group G and viridans streptococci), enterococci (including vancomycin-susceptible and vancomycin-resistant strains such as Enterococcus faecalis and E. faecium), Clostridium difficile, C. clostridiiforme, C. innocuum, C. perfringens, C. ramosum, Haemophilus influenzae, Listeria monocytogenes, Corynebacterium jeikeium, Bifidobacterium spp., Eubacterium aerofaciens, E. lentum, Lactobacillus acidophilus, L. casei, L. plantarum, Lactococcus spp., Leuconostoc spp., Pediococcus, Peptostreptococcus anaerobius, P. asaccarolyticus, P. magnus, P. micros, P. prevotii, P. productus, Propionibacterium acnes, Actinoniyces spp., Moraxella spp. (including M. catarrhalis) and Escherichia spp. (including E. coli).

In a preferred embodiment, the antibacterial activity of compounds of Formula I or compounds of any of Formula F1-F22 against classically “resistant” strains is comparable to that against classically “susceptible” strains in in vitro experiments. In another preferred embodiment, the minimum inhibitory concentration (MIC) value for compounds according to this invention, against susceptible strains, is typically the same or lower than that of vancomycin or daptomycin. Thus, in a preferred embodiment, a compound of this invention or a pharmaceutical composition thereof is administered according to the methods of this invention to a patient who exhibits a bacterial infection that is resistant to other compounds, including vancomycin or daptomycin. In addition, unlike glycopeptide antibiotics, depsipeptide compounds such as those disclosed in the present invention, exhibit rapid, concentration-dependent bactericidal activity against gram-positive organisms. Thus, in a preferred embodiment, a compound according to this invention or a pharmaceutical composition thereof is administered according to the methods of this invention to a patient in need of rapidly acting antibiotic therapy.

The method of the instant invention may be used for any bacterial infection of any organ or tissue in the body. In a preferred embodiment, the bacterial infection is caused by gram-positive bacteria. These organs or tissue include, without limitation, skeletal muscle, skin, bloodstream, kidneys, heart, lung and bone. The method of the invention may be used to treat, without limitation, skin and soft tissue infections, bacteremia and urinary tract infections. The method of the invention also may be used to treat mixed infections that comprise different types of gram-positive bacteria, or which comprise both gram-positive and gram-negative bacteria. These types of infections include intra-abdominal infections and obstetrical/gynecological infections. The method of the invention also may be used to treat an infection including, without limitation, endocarditis, nephritis, septic arthritis, intra-abdominal sepsis, bone and joint infections. and osteomyelitis. In a preferred embodiment, any of the above-described diseases may be treated using compounds according to this invention or pharmaceutical compositions thereof.

The method of the present invention may also be practiced while concurrently administering one or more other antimicrobial agents, such as antibacterial agents (antibiotics) or antifungal agents. In one aspect, the method may be practiced by administering more than one compound according to this invention. In another embodiment, the method may be practiced by administering a compound according to this invention with a lipopeptide compound, such as daptomycin or the lipopeptide compounds described, for example in U.S. Pat. Nos. 6,911,525; and 6,794,490 and in International Patent Applications WO01/44272; WO01/44274; WO01/44271 and WO03/014147.

Antibacterial agents and classes thereof that may be co-administered with a compound according to the invention include, without limitation, penicillins and related drugs, carbapenems, cephalosporins and related drugs, aminoglycosides, bacitracin, gramicidin, mupirocin, chloramphenicol, thiamphenicol, fusidate sodium, lincomycin, clindamycin, macrolides, novobiocin, polymyxins, rifamycins, spectinomycin, tetracyclines, vancomycin, teicoplanin, streptogramins, anti-folate agents including sulfonamides, trimethoprim and its combinations and pyrimethamine, synthetic antibacterials including nitrofurans, methenamine mandelate and methenamine hippurate, nitroimidazoles, quinolones, fluoroquinolones, isoniazid, ethambutol, pyrazinamide, para-aminosalicylic acid (PAS), cycloserine, capreomycin, ethionamide, prothionamide, thiacetazone, viomycin, everninomycin, glycopeptide, glycylcylcline, ketolides, oxazolidinone; imipenen, amikacin, netilmicin, fosfomycin, gentamicin, ceftriaxone, ZIRACIN®, LY 333328, CL 331002, HMR 3647, ZYVOX®, SYNERCID®, aztreonam metronidazole, epiroprim, OCA-983, GV-143253, sanfetrinem sodium, CS-834, biapenem, A-99058.1, A-165600, A-179796, KA 159, dynemicin A, DX8739, DU 6681; cefluprenam, ER 35786, cefoselis, sanfetrinem celexetil, HGP-31, cefpirome, HMR-3647, RU-59863, mersacidin, KP 736, rifalazil; AM 1732, MEN 10700, lenapenem, BO 2502A, NE-1530, PR 39, K130, OPC 20000, OPC 2045, veneprim, PD 138312, PD 140248, CP 111905, sulopenem, ritipenam acoxyl, RO-65-5788, cyclothialidine, Sch-40832, SEP-132613, micacocidin A, SB-275833, SR-15402, SUN A0026, TOC 39, carumonam, cefozopran, cefetamet pivoxil, and T 3811.

Antifungal agents that may be co-administered with a compound according to the invention include, without limitation, caspofungen, voriconazole, sertaconazole, IB-367, FK-463, LY-303366, Sch-56592, sitafloxacin, DB-289 polyenes, such as amphotericin, nystatin, primaricin; azoles, such as fluconazole, itraconazole, and ketoconazole; allylamines, such as naftifine and terbinafine; and anti-metabolites such as flucytosine. Other antifungal agents include without limitation, those disclosed in Fostel, et al., 2000, Drug Discovery Today 5: 25-32, herein incorporated by reference. Fostel et al. discloses antifungal compounds including corynecandin, Mer-WF3010, fusacandins, artrichitin/LL 15G256, sordarins, cispentacin, azoxybacillin, aureobasidin and khafrefungin.

A compound according to this invention may be administered according to this method until the bacterial infection is eradicated or reduced. In one embodiment, a compound of Formula I or a compound of any of Formulas F1-F22 is administered for a period of time from 2 days to 6 months. In a preferred embodiment, a compound of Formula I or a compound of any of Formulas F1-F22 is administered for 7 to 56 days. In a more preferred embodiment a compound of Formula I or a compound of any of Formulas F1-F22 is administered for 7 to 28 days. In an even more preferred embodiment, a compound of Formula I or a compound of any of Formulas F1-F22 is administered for 7 to 14 days. A compound of Formula I or or a compound of any of Formulas F1-F22 may be administered for a longer or shorter time period if it is so desired.

The instant invention provides antibacterial compositions or formulations comprising, in one embodiment, compounds of Formula I or compounds of any of Formula F1-F22, or salts thereof. In one embodiment the antibacterial compositions may be contained in an aqueous solution. In another embodiment the aqueous solution may be buffered. In another embodiment the buffer may have an acidic, neutral, or basic pH.

### Preparation of Novel Depsipeptides

### 1. Synthetic Processes

In one embodiment of the invention, the compounds of Formula I or Formula F1-F22 may be prepared using solid support chemistry. Three preferred methods, Methods A-C, produce resin bound linear precursor nn3, nn3a or nn3b.

As outlined in Scheme I, Method A utilizes a resin-bound 7 amino acid-derived polypeptide fragment, nn1, and a six amino acid-derived polypeptide fragment, nn2. This method is referred to as a “7+6 fragment synthesis”.

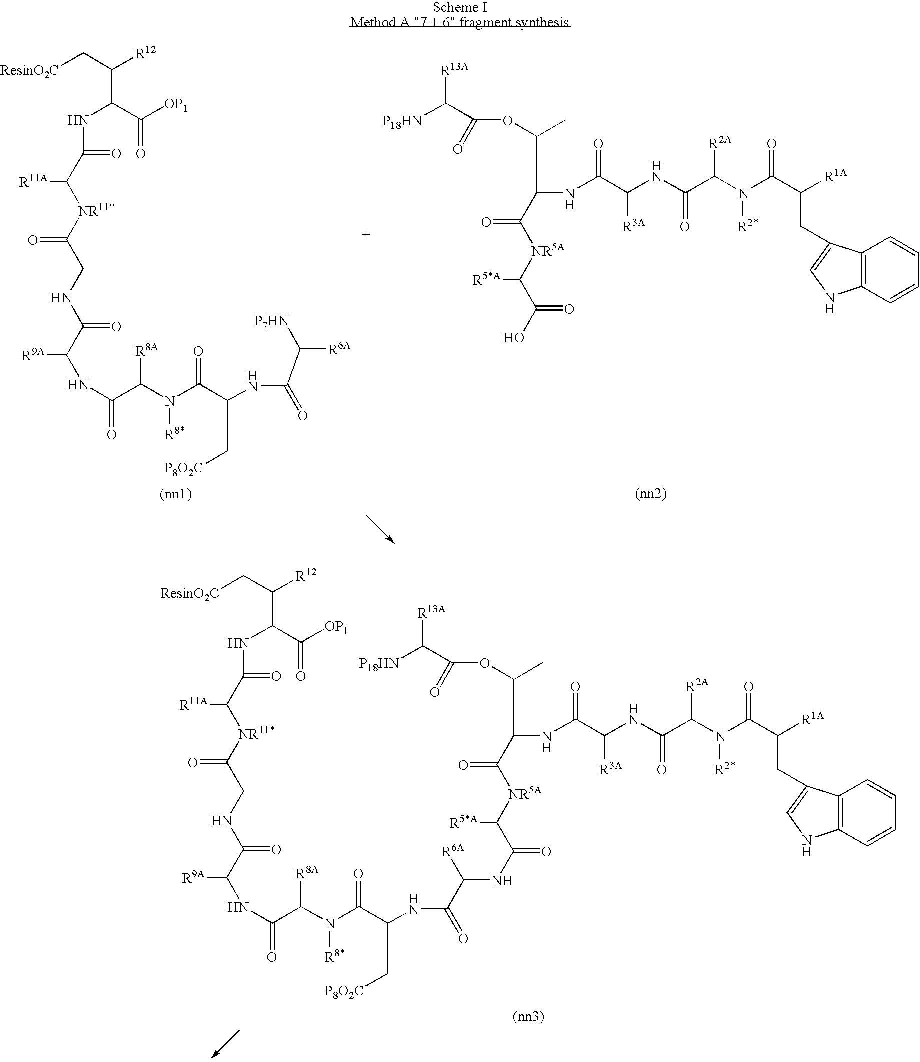

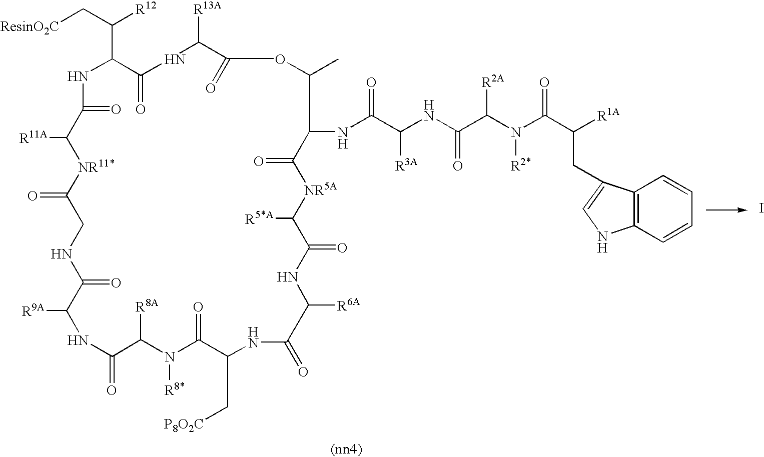

Alternatively, as described in Scheme II, Method B utilizes a resin-bound 6 amino acid-derived polypeptide fragment, nn1a, and a seven amino acid-derived polypeptide fragment, nn2a. This method is referred to as a “6+7 fragment synthesis”.

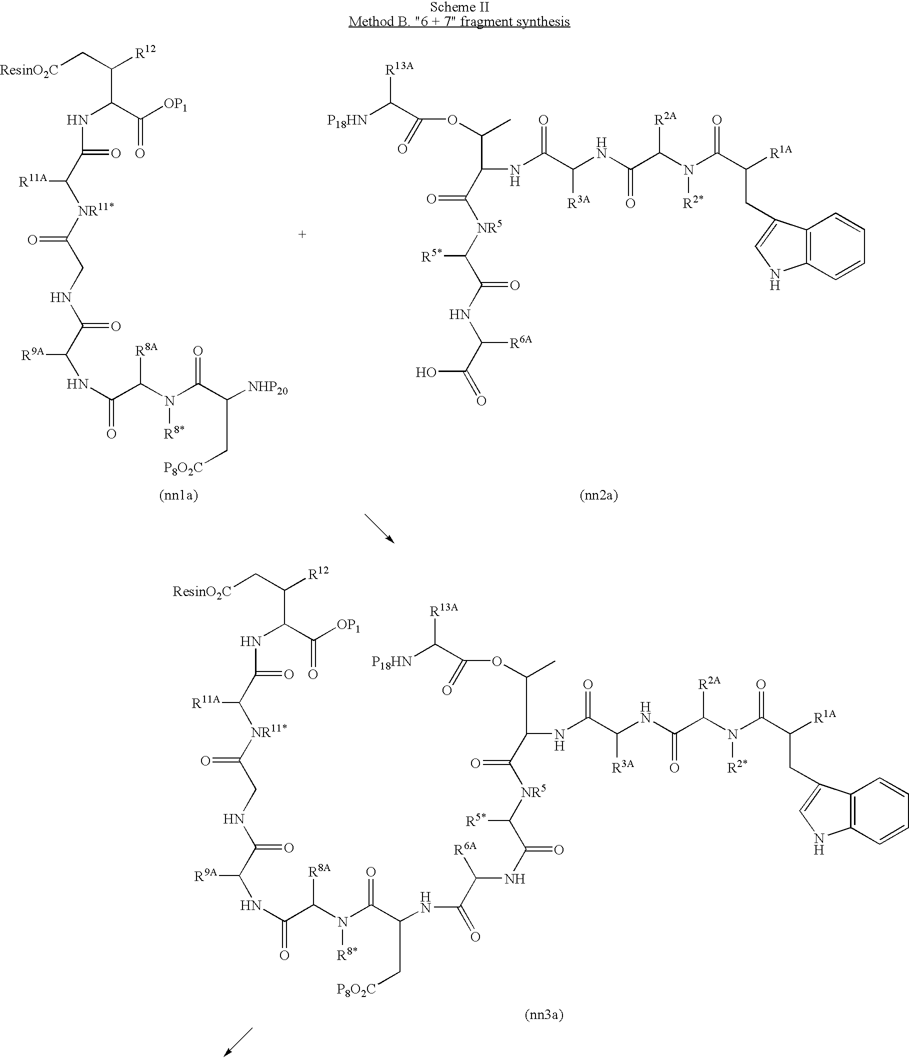

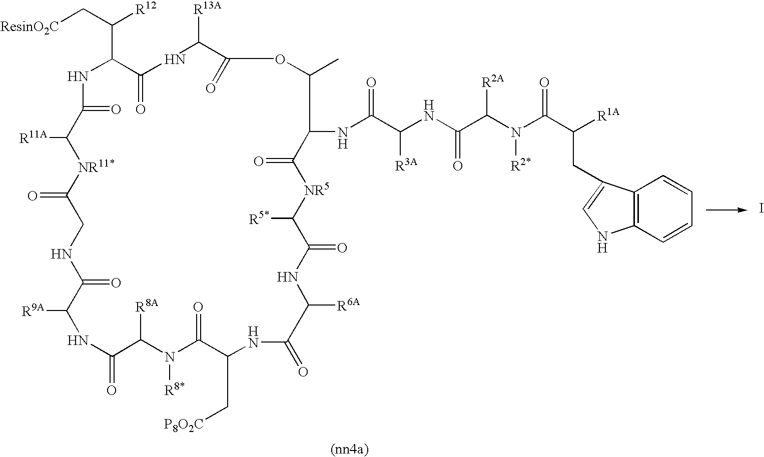

Another method, Method C, utilizes a 6 amino acid derived polypeptide, a resin bound-amino acid, and a second 6 amino acid derived polypeptide. This method is referred to as a “1+6+6 fragment synthesis”. 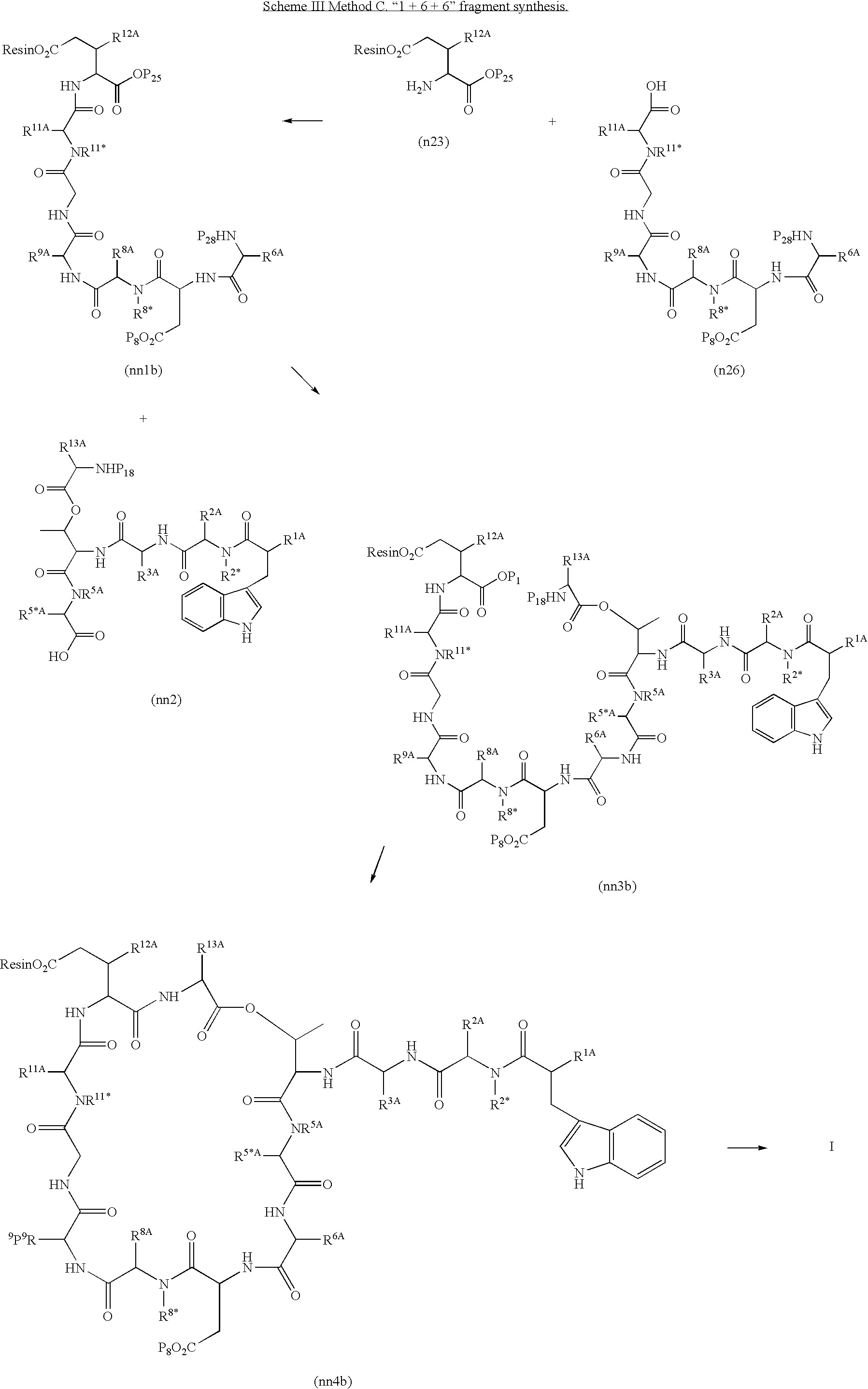
Solid Support Synthesis of Depsipeptide Compounds Methods A: 7+6 Fragment Synthesis

The depsipeptide compounds of Formula I may be synthesized on a solid support as outlined in Scheme IV, Scheme V and Scheme VI as follows.

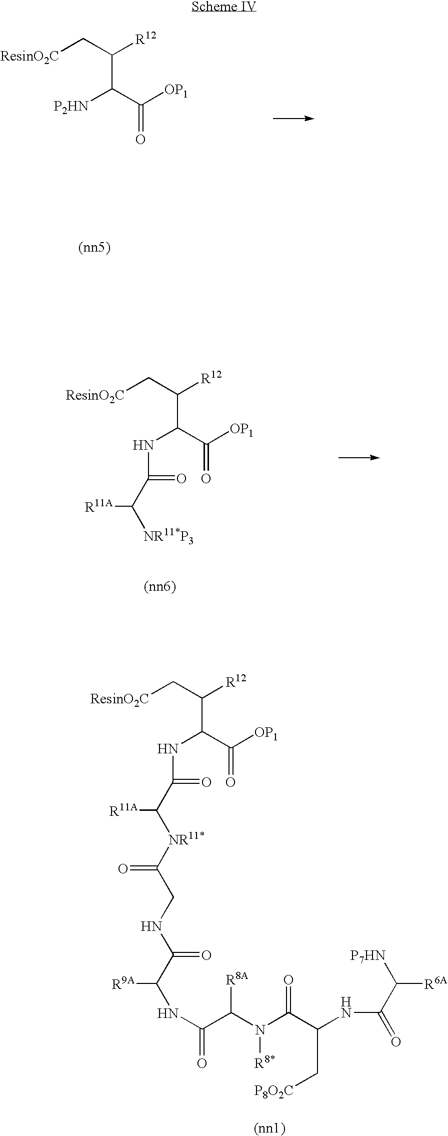

In the first step, a protected glutamic acid-derivative such as commercially available N-a-Fmoc-L-glutamic acid a-allyl ester or N-Fmoc-L-3-methyl glutamic acid a-allyl ester (See Examples 1-68 and 1-69, vide infra) is coupled to a resin to give Compound nn5, wherein R¹² is as defined previously. A resin or solid support, such as, but not limited to, Wang, HMPA, Safety Catch, Rink Acid, 2-chlorotrityl-chloride resin, trityl-chloride resin, 4-methyltrityl-chloride resin, 4-methoxytrityl-chloride resin or PAM resin may be used in this reaction. Protecting groups P₁ and P₂ are chosen so that they may be removed independently of one another and without effecting cleavage of the peptide from the resin. Examples of protecting groups can be found in “Protecting Groups in Organic Synthesis” by Theodora W. Greene, (vide supra), hereafter “Greene”, incorporated herein by reference. A protecting group combination, such as, but not limited to P₁ is allyl ester and P₂ is Fmoc is suitable for this reaction.

Deprotection of the amine of Compound nn5, followed by coupling of the free amino with an amino acid or a protected amino acid affords Compound nn6, wherein P₃ is a protecting group that can be removed independently of P₁ and without effecting cleavage of the peptide from the resin; R^{11A} is an amino acid side chain, a protected amino acid side chain, methyl, CH₂—OP₄, or CH₂—CONHP₅; each of P₄ and P₅ is independently a suitable protecting group and each of P₁ and R¹¹* is as defined previously. This peptide coupling process, i.e., deprotection of the alpha-amino group, followed by coupling to a protected amino acid, is repeated until the desired number of amino acids has been coupled to the resin. In Scheme IV, a total of seven amino acids have been coupled to give compound nn1 wherein, R^{6A} is methyl or 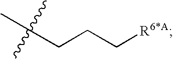
R^{6*A} is a protected amino, monosubstituted amino, disubstituted amino, acylamino, ureido, guanidino, carbamoyl, sulfonamino, thioacylamino, thioureido, iminoamino, or phosphonamino, provided that R^{6*A} is compatible with the conditions required to remove the resin from the peptide;

R^{8A} is an amino acid side chain, a protected amino acid side chain, methyl, CH₂—OP₆, CH₂—CONHP₅* or 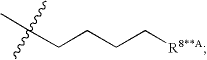
wherein each of P₅* and P₆ is independently a suitable protecting group; wherein R^{8**A} is a protected amino, monosubstituted amino, disubstituted amino, acylamino, ureido, guanidino, carbamoyl, sulfonamino, thioacylamino, thioureido, iminoamino, or phosphonamino, provided that R^{8**A} is compatible with the conditions required to remove the resin from the peptide;
wherein R^{9A} is 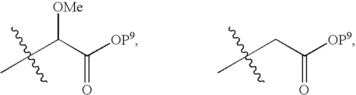
or an amino acid side chain substituted with at least one carboxylic acid group of the formula, 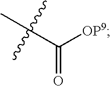
P₇ is a protecting group that can be removed independently of P₁ without effecting cleavage of the peptide from the resin; each of P₈ and P₉ is independently a suitable protecting group such that P₁ and P₇ may be removed independently of each of P₈ and P₉ and that each of P₈ and P₉ is cleaved upon cleavage from the resin; and P₁, R⁸*, R^{9A}, R¹¹*, R^{11A} and R¹² are as defined previously.

A second peptide is coupled to a resin in a similar fashion, as outlined in Scheme V.

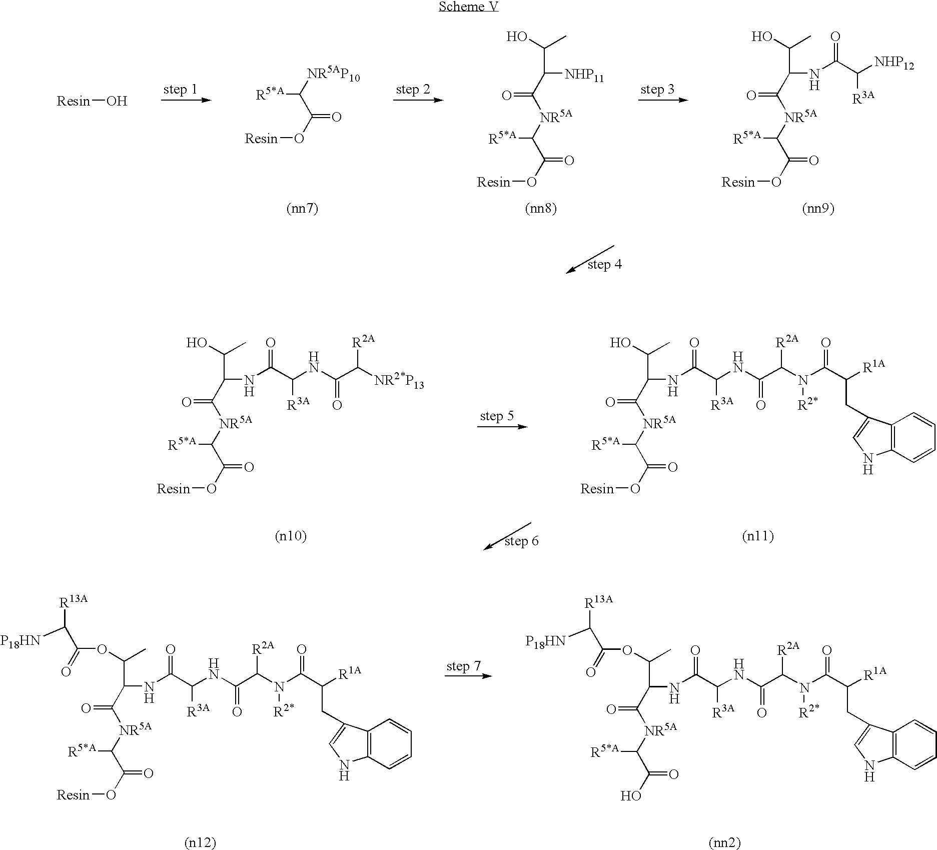

In step 1, an N-protected-glycine, such as commercially available Fmoc-N-glycine, is coupled to a resin to give Compound nn7 wherein R^{5A} and R^{5*A} are independently hydrido and P₁₀ is a protecting group chosen so that it may be removed without effecting cleavage of the peptide from the resin. The choice of resin used in step 1 is dependent upon the nature of the amino acid that is coupled in steps 2-6. If the amino acid side chains contain protecting groups, a resin must be chosen such that the protecting groups remain intact when the resin is removed from the peptide in step 7. Resins that can be cleaved while preserving the protecting groups of peptides include, but are not limited to, Safety Catch, Rink Acid, 2-chlorotrityl-chloride resin, trityl-chloride resin, 4-methyltrityl-chloride resin, 4-methoxytrityl-chloride resin or PAM resin.

Deprotection of the protected amino of Compound nn7, followed by coupling of the free amino with n14 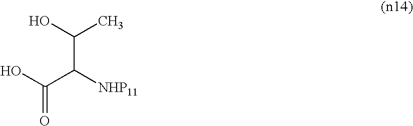
affords Compound nn8, wherein P₁₁ is a protecting group chosen so that it may be removed without effecting cleavage of the peptide from the resin. This peptide coupling process, i.e., deprotection of the alpha-amino group, followed by coupling to a protected amino acid, is repeated until the desired number of amino acids has been coupled to the resin. In Scheme V, five amino acids have been coupled to give Compound n11 wherein R^{1A} is a protected amino, monosubstituted amino, disubstituted amino, acylamino, ureido, guanidino, carbamoyl, sulfonamino, thioacylamino, thioureido, imino amino, or phosphonamino, provided that R^{1A} is compatible with the conditions required to remove the resin from the peptide; R^{2A} is an amino acid side chain, a protected amino acid side chain, CH₂—CH₂—CO₂P₁₄, or CH₂—CONHP₁₅; R^{3A} is CH₂—CO₂P₁₆, CH(OP₁₇)CONH₂, CH₂CONH₂, a non-protienogenic amino acid side chain, or a protected non-proteinogenic amino acid side chain; each of P₁₂ and P₁₃ is a protecting group chosen so that it may be removed without effecting cleavage of the peptide from the resin; each P₁₄, P₁₅, P₁₆ and P₁₇ is independently a suitable protecting group; and R²*, R^{5A} and R^{5*A} is as previously defined.

Compound n11 is coupled with 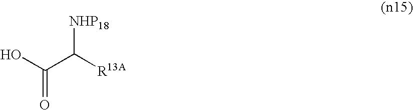
to give Compound n12, wherein P₁₈ is a suitable protecting group and R^{13A} is CH(CH₃)₂, CH(CH₂CH₃)CH₃,

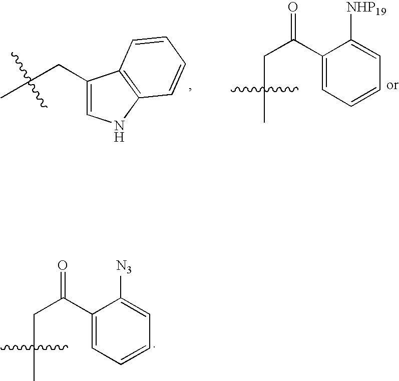

The peptide n12 is then removed from the resin to give compound nn2 wherein P₁₉ is a suitable protecting group.

Coupling of the peptide fragments nn1 and nn2 is outlined in Scheme VI.

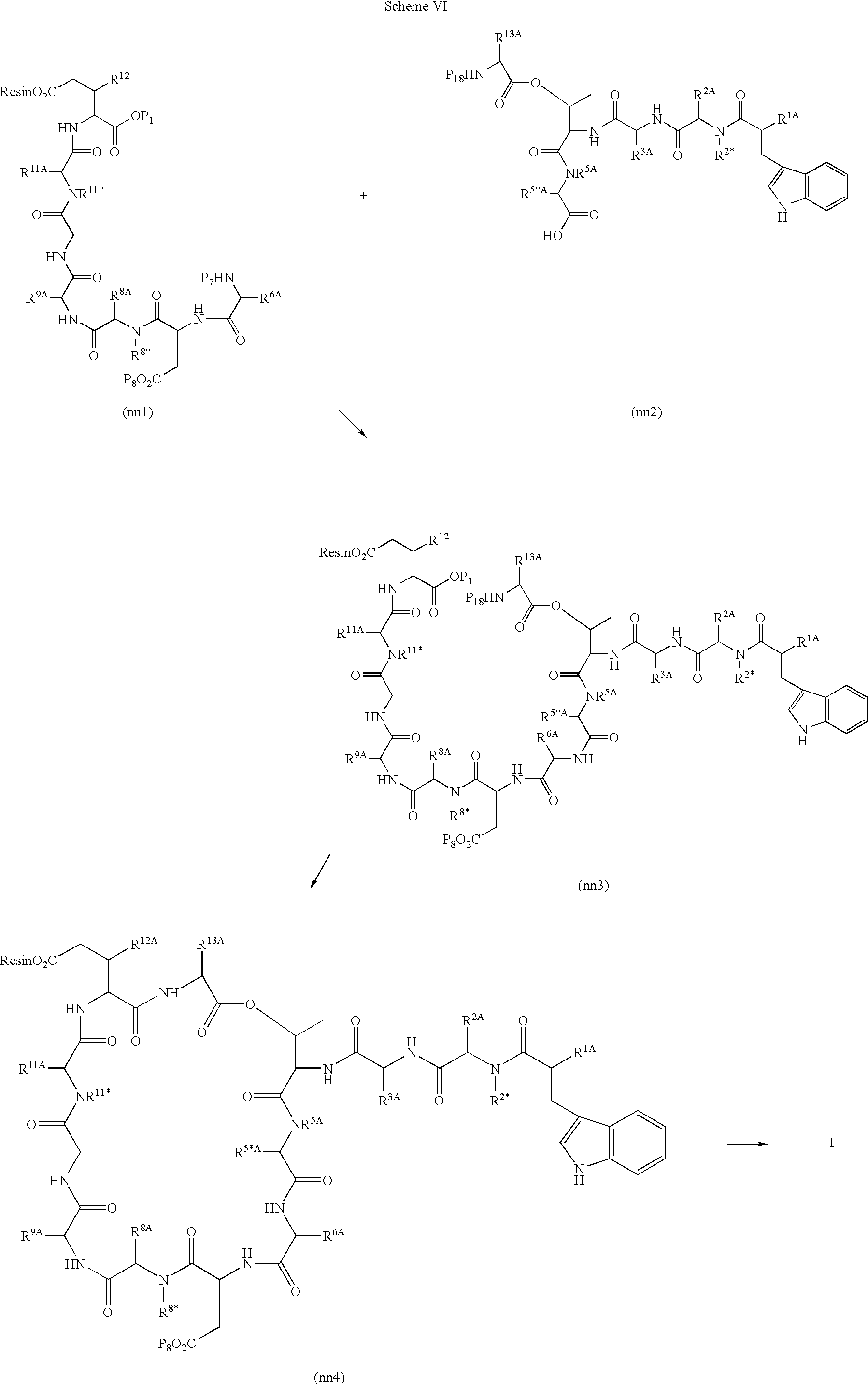

The peptide fragments nn1 and nn2 are coupled to yield the resin bound peptide nn3 wherein, R^{1A}, R²*, R^{2A}, R^{3A}, R^{5A}, R^{5*A}, R^{6A}, R⁸*, R^{8A}, R^{9A}, R^{11A}, R¹¹*, R¹², R^{13A}, P₁, P₈, P₁₈ are as previously defined Deprotection of the P₁ and P₁₈ protecting groups, followed by cyclization affords a resin-bound depsipeptide nn4 wherein, R^{1A}, R²*, R^{2A}, R^{3A}, R^{5A}, R^{5*A}, R^{6A}, R⁸*, R^{8A}, R^{9A}, R^{11A}, R¹¹*, R¹², R^{13A}, and P₈ are as previously defined. Cleavage of the depsipeptide from the resin and deprotection of any remaining protecting groups yields compounds of Formula I.

Solid Support Synthesis of Depsipeptide Compounds Method B:

6+7 Fragment Synthesis

The depsipeptide compounds of Formula I may be synthesized on a solid support as described in Schemes VII, VIII and IX.

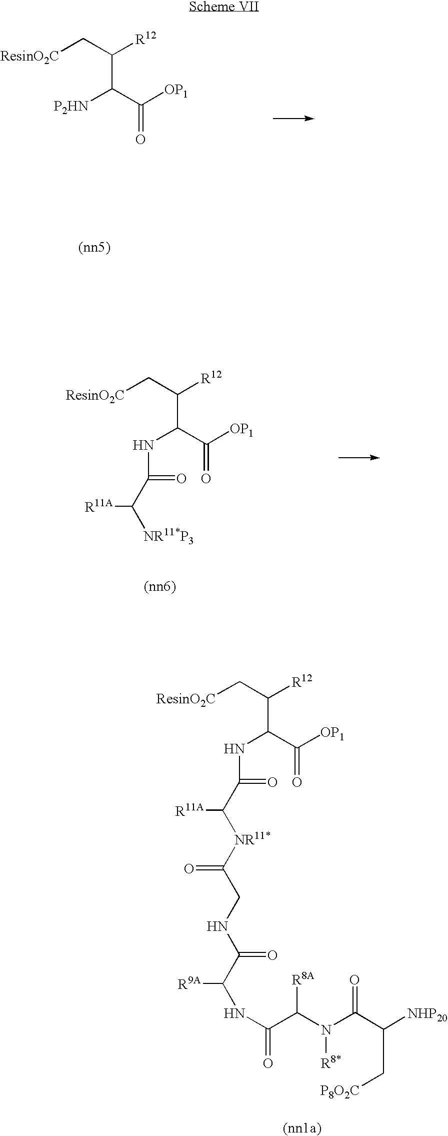

Compound nn6 is prepared as described in Method A. The peptide coupling process (vide supra), i.e., deprotection of the alpha-amino group, followed by coupling to a protected amino acid, is repeated until the desired number of amino acids has been coupled to the resin. In Scheme VII, a total of six amino acids have been coupled to give compound nn1a wherein, R⁸*, R^{8A}, R^{9A}, R¹¹*, R^{11A}, R¹², P₁, and P₈ are as defined previously and P₂₀ is a protecting group that can be removed independently of P₁ and without effecting cleavage of the peptide from the resin, such as P₁ is allyl and P₂₀ is Fmoc.

A second peptide is coupled to a resin in a similar fashion, as outlined in Scheme VIII.

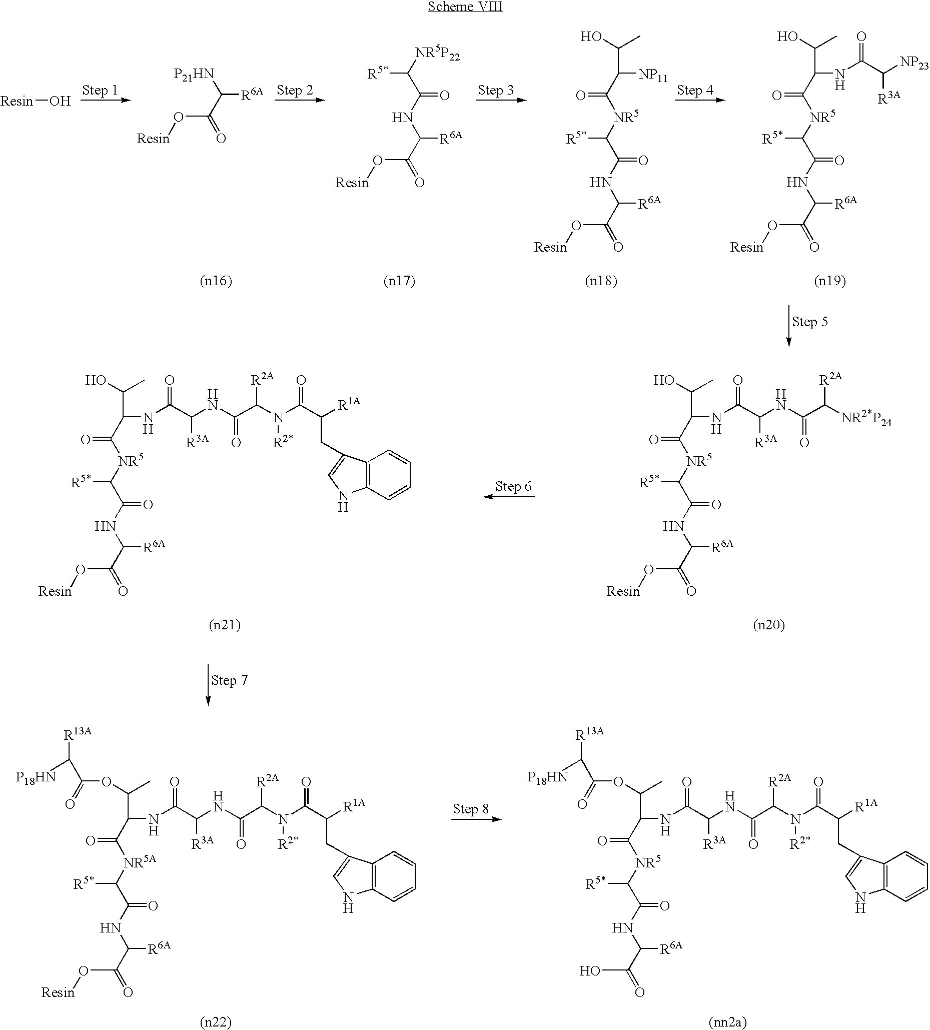

In step 1, a N-protected-amino acid is coupled to a resin to give Compound n16 wherein P₂₁ is a protecting group that can be removed without effecting cleavage of the peptide from the resin and R^{6A} is as defined previously. The choice of resin used in step 1 is dependent upon the nature of the amino acid that is coupled in steps 2-6. If the amino acid side chains contain protecting groups, a resin must be chosen such that the protecting groups remain intact when the resin is removed from the peptide in step 8. Resins that can be cleaved while preserving the protecting groups of peptides include, but are not limited to, Safety Catch, Rink Acid, 2-chlorotrityl-chloride resin, trityl-chloride resin, 4-methyltrityl-chloride resin, 4-methoxytrityl-chloride resin or PAM resin.

Deprotection of the protected amino of Compound n16, followed by coupling of the free amino with a second protected amino acid affords Compound n17 wherein P₂₂ is a protecting group that can be removed without effecting cleavage of the peptide from the resin; and R⁵, R⁵*, and R⁶ are as defined previously.

Deprotection of the protected amino of Compound n17, followed by coupling of the free amino with n14 (vide supra) affords Compound n18, wherein, R⁵, R⁵*, R^{6A} and P₉ are as described previously. The peptide coupling process, i.e., deprotection of the alpha-amino group, followed by coupling to a protected amino acid, is repeated until the desired number of amino acids has been coupled to the resin. In Scheme VIII, six amino acids have been coupled to give Compound n21, wherein each of P₂₃ and P₂₄ is a protecting group that can be removed without effecting cleavage of the peptide from the resin; R^{1A}, R^{2A}, R²*, R^{3A}, R⁵, R⁵*, and R^{6A} are as described previously.

Compound n21 is coupled with n15 (vide supra) to give Compound n22, wherein R^{1A}, R^{2A}, R²*, R^{3A}, R⁵, R⁵*, R^{6A}, R^{13A} and P₁₈ are as described previously.

The peptide n22 is then removed from the resin to give compound nn2a, wherein R^{1A}, R^{1A}, R²*, R^{3A}, R⁵, R⁵*, R^{6A}, R^{13A} and P₁₈ are as described previously.

Coupling of the peptide fragments nn1a and nn2a is outlined in Scheme IX.

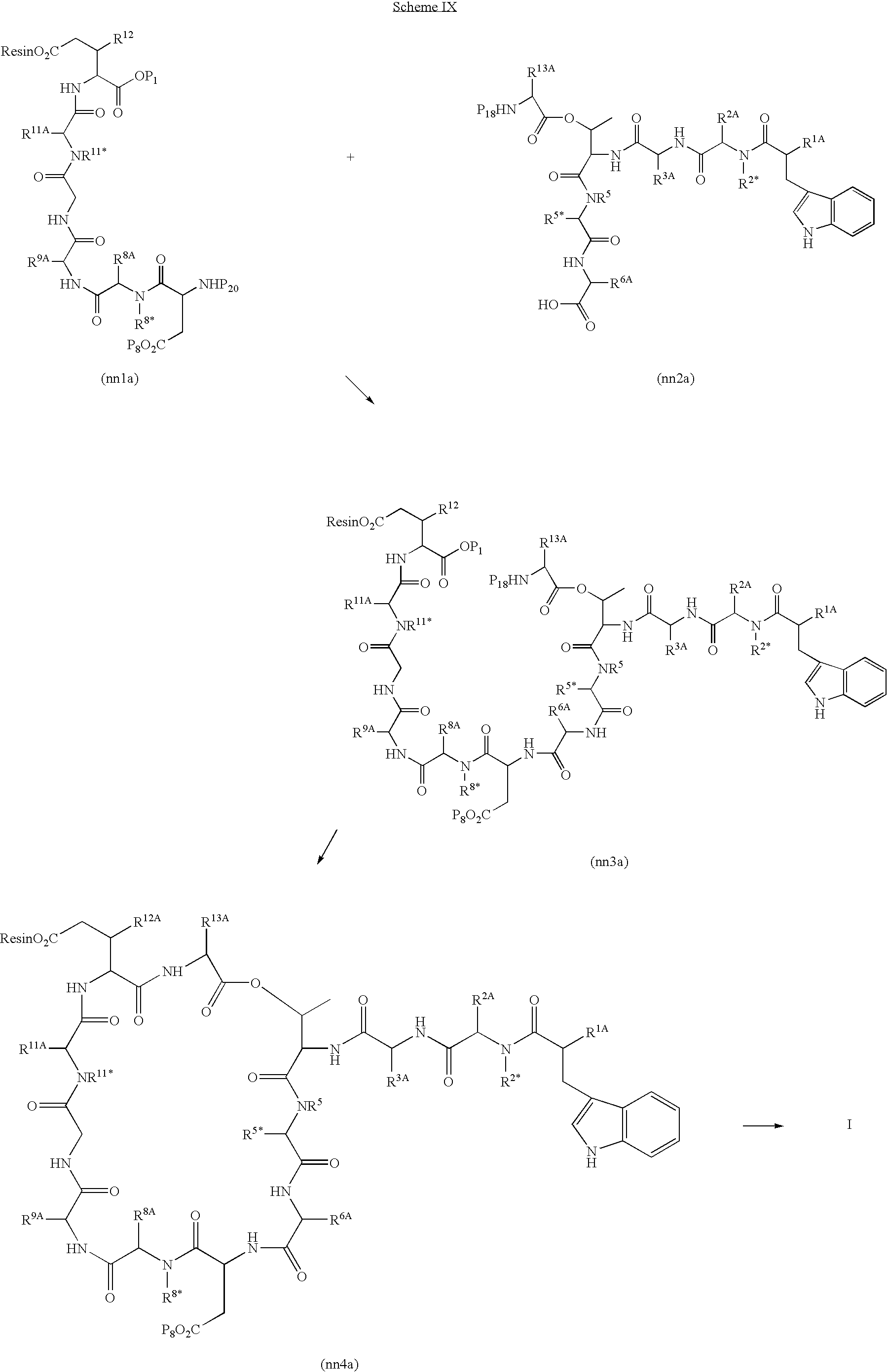

The peptide fragments nn1a and nn2a are coupled to yield the resin bound peptide nn3a wherein R^{1A}, R^{2A}, R²*, R^{3A}, R⁵, R⁵*, R^{6A}, R⁸*, R^{8A}, R^{9A}, R¹¹*, R^{11A}, R¹², R^{13A}, P₁, P₈, P₉ and P₁₈ are as described previously.

Deprotection of the P₁ and P₁₈ protecting groups, followed by cyclization affords a resin-bound depsipeptide nn4a, wherein R^{1A}, R^{2A}, R²*, R^{3A}, R⁵, R⁵*, R^{6A}, R⁸*, R^{8A}, R^{9A}, R¹¹*, R^{11A}, R¹², R^{13A}, and P₈ are as described previously.

Cleavage of the depsipeptide from the resin and deprotection of any remaining protecting groups yields compounds of Formula I.

Solid Support Synthesis of Depsipeptide Compounds Method C 1+6+6 Fragment Synthesis.

In an alternative embodiment of the invention, the depsipeptide compounds of Formula I may be synthesized as described in Schemes X-XII.

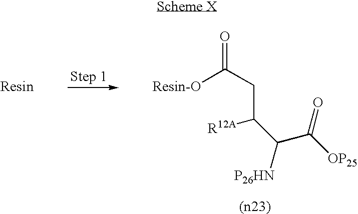

In step 1, a protected-β-methyl glutamic acid derivative such as commercially available N-a-Fmoc-L-glutamic acid a-allyl ester or N-Fmoc-L-3-methyl glutamic acid a-allyl ester (See Examples 1-68 and 1-69, vide infra) is coupled to a resin to give Compound n23 wherein R^{12A} is methyl. A resin or solid support, such as, but not limited to, Wang, HMPA, Safety Catch, Rink Acid, 2-chlorotrityl-chloride resin, trityl-chloride resin, 4-methyltrityl-chloride resin, 4-methoxytrityl-chloride resin or PAM resin may be used in this reaction. Protecting groups P₂₅ and P₂₆ are chosen so that they can be removed independently of one another and without effecting cleavage of the peptides from the resin. A protecting group combination, such as, but not limited to P₂₅ is allyl ester and P₂₆ is Fmoc is suitable for this reaction.

A second peptide is coupled to a resin in a similar fashion, as outlined in Scheme XI.

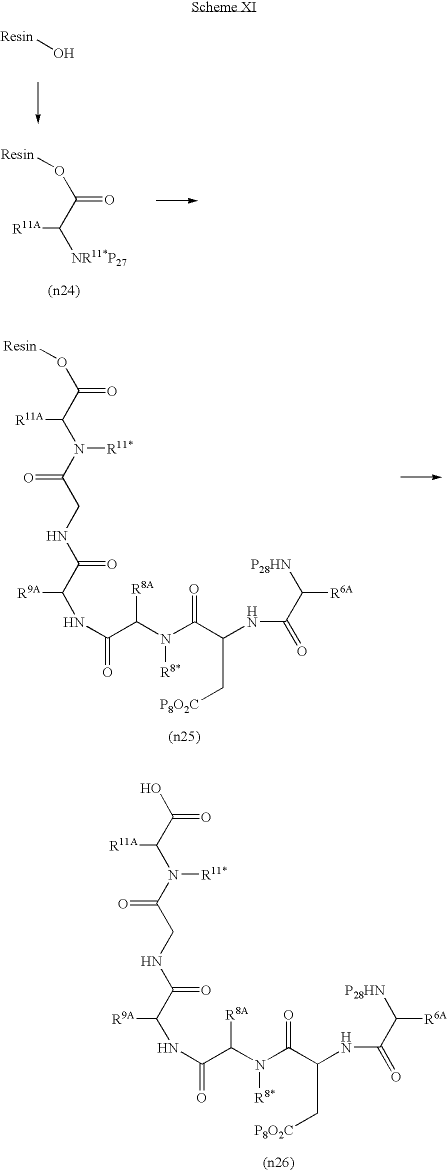

In step 1, a protected amino acid is coupled to a resin to give Compound n24, wherein P₂₇ is a protecting group that can be removed without effecting cleavage of the peptide from the resin; R¹¹* and R^{11A} are as previously defined. The choice of resin used in the first step is dependent upon the nature of the amino acid that is coupled in the proceeding steps. If the amino acid side chains contain protecting groups, a resin must be chosen such that these protecting groups remain intact when the peptide is removed from the resin. Resins that can be cleaved while preserving the protecting groups of peptides include, but are not limited to, Safety Catch, Rink Acid, 2-chlorotrityl-chloride resin, trityl-chloride resin, 4-methyltrityl-chloride resin, 4-methoxytrityl-chloride resin or PAM resin.

This peptide coupling process, i.e., deprotection of the alpha-amino group, followed by coupling to a protected amino acid, is repeated until the desired number of amino acids has been coupled to the resin. In Scheme XI, a total of six amino acids have been coupled to give compound n25 wherein, P₂₈ is a protecting group that can be removed without effecting cleavage of the peptide from the resin; R^{6A}, R⁸*, R^{8A}, R^{9A}, R¹¹*, R^{11A}, and P₈ are as previously defined.

Cleavage of the peptide from the resin affords compound n26. Coupling of the 3 peptide fragments is outlined in Scheme XII.

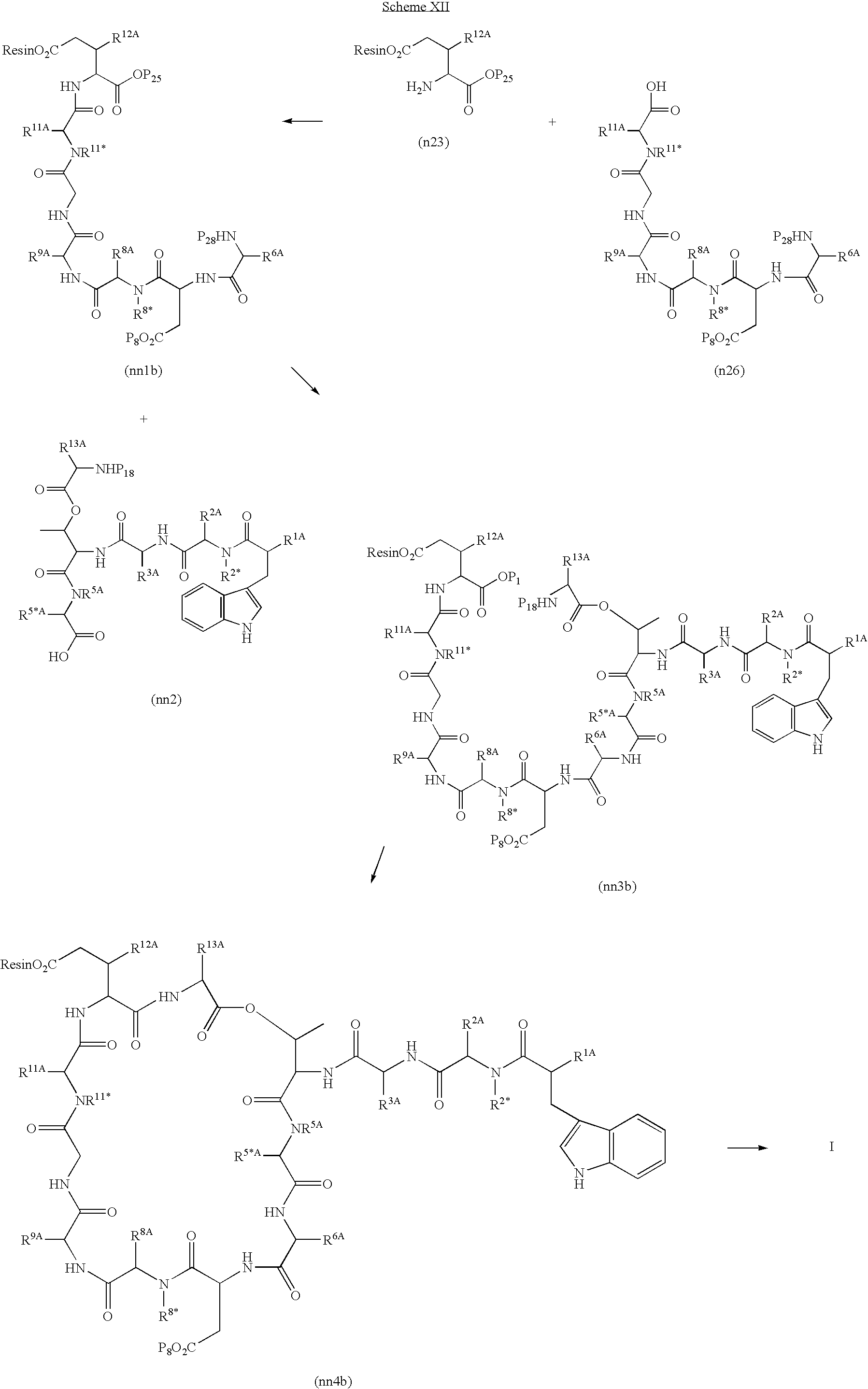

The resin bound 3-methylglutamate n23, where R^{12A} is as described previously is deprotected to give the free amine then coupled to fragment n26 to give resin bound fragment nn1b, wherein R^{11A}, R¹¹*, R^{9A}, R^{8A}, R⁸*, R^{6A}, P₈, P₂₅, and P₂₈, are as previously described. This is then coupled to the previously described fragment nn2, to give nn3b wherein R^{1A}, R^{2A}, R²*, R^{3A}, R^{5*A}, R^{5*A}, R^{6A}, R⁸*, R^{8A}, R^{9A}, R¹¹*, R^{11A}, R^{12A}, R^{13A}, P₁, P₈, and P₁₈ are as described previously. Deprotection and cyclization as described in Methods A affords a resin-bound depsipeptide nn4b wherein R^{1A}, R^{2A}, R²*, R^{3A}, R^{5*A}, R^{5*A}, R^{6A}, R⁸*, R^{8A}, R^{9A}, R¹¹*, R^{11A}, R^{12A}, R^{13A}, and P₈ are as described previously. Cleavage of the depsipeptide from the resin followed by deprotection of any remaining protecting groups yields compounds of Formula I.

Following the synthetic schemes above (Schemes IV-XII), it is understood that both the amino acid amino group and the amino acid side chain functional groups must be orthogonally protected prior to attaching them to the growing peptide chain. Suitable protecting groups can be any protecting group useful in peptide synthesis. Such pairings of protecting groups are well known. See, e.g., “Synthesis Notes” in the Novabiochem Catalog and Peptide Synthesis Handbook, 1999, pages S1-S93 and references cited therein.

It will also be understood by those skilled in the art that the choice of protecting group on the amino acid side chain functional groups will either result or not result in the protecting group being cleaved concomitantly with the peptide's final cleavage from the resin, which will give the natural amino acid functionality or a protected derivative thereof, respectively. When the protecting groups are not concomitantly cleaved when the depsipeptide is cleaved from the resin, additional deprotection may be necessary.

It would be clear to one of skill in the art that the linear precursor nn3 nn3a or nn3b and hence intermediate nn4 nn4a and nn4b and final product I can be obtained not only by Methods A-C as described above, but also, by combining any two fragment pairs. These fragment pairs can be envisioned by fragmenting the compound of Formula I between any two amino acids in the sequence, i.e. 1+12, 2+11, 3+10, etc.

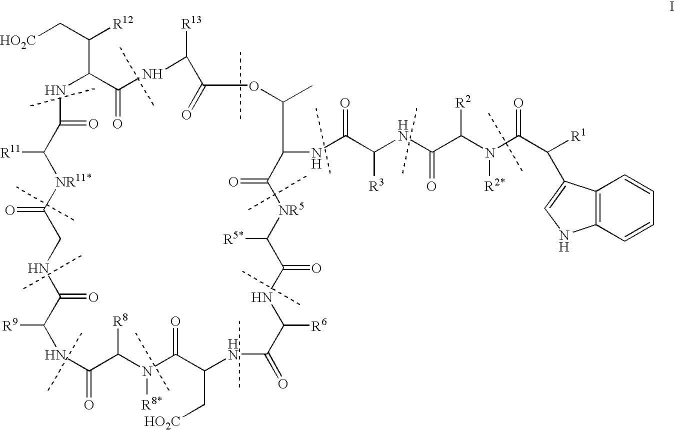

Alternatively, the compounds can be formed by linear assembly prior to ester formation by the methods described in U.S. Pat. Nos. 6,911,525 and 6,794,490, and International Patent Application Numbers WO01/44272, WO01/44274, WO01/44271 and WO03/014147. Alternatively, the compounds can be formed by assembly of multiple fragments.

Although the methods described above employ resin chemistry, the methods would also be suitable for solution-phase peptide chemistry.

Alternatively, the compounds of the present invention can be formed by the methods described in International Patent Application Number WO2005/012541.

### 2. Biosynthetic Process

Non-Ribosomal Peptide Synthetases Pathways

Bacteria, including actinomycetes, and fungi synthesize a diverse array of low molecular weight peptide and polyketide compounds (approx. 2-48 residues in length). The biosynthesis of these compounds is catalyzed by non-ribosomal peptide synthetases (NRPSs) and by polyketide synthetases (PKSs). The NRPS process, which does not involve ribosome-mediated RNA translation according to the genetic code, is capable of producing peptides that exhibit enormous structural diversity, compared to peptides translated from RNA templates by ribosomes. These include the incorporation of D- and L-amino acids and hydroxy acids; variations within the peptide backbone which form linear, cyclic or branched cyclic structures; and additional structural modifications, including oxidation, acylation, glycosylation, N-methylation and heterocyclic ring formation. Many non-ribosomally synthesized peptides have been found which have useful pharmacological (e.g., antibiotic, antiviral, antifungal, antiparasitic, siderophore, cytostatic, immunosuppressive, anti-cholesterolemic and anticancer), agrochemical or physicochemical (e.g., biosurfactant) properties.

Non-ribosomally synthesized peptides are assembled by large (e.g., about 200-2000 kDa), multifunctional NRPS enzyme complexes comprising one or more subunits. Examples include daptomycin, A54145, vancomycin, echinocandin and cyclosporin. Likewise, polyketides are assembled by large multifunctional PKS enzyme complexes comprising one or more subunits. Examples include erythromycin, tylosin, monensin and avermectin. In some cases, complex molecules can be synthesized by mixed PKS/NRPS systems. Examples include rapamycin, bleomycin and epothilone.

An NRPS usually consists of one or more open reading frames that make up an NRPS complex. The NRPS complex acts as a protein template, comprising a series of protein biosynthetic units configured to bind and activate specific building block substrates and to catalyze peptide chain formation and elongation. (See, e.g., Konz and Marahiel, 1999, Chem. Biol. 6: 39-48 and references cited therein; von Döhren et al., 1999, Chem. Biol. 6: 273-279, and references cited therein; and Cane and Walsh, 1999, Chem. Biol. 6: 319-325, and references cited therein—each hereby incorporated by reference in its entirety). Each NRPS or NRPS subunit comprises one or more modules. A “module” is defined as the catalytic unit that incorporates a single building block (e.g., an amino acid) into the growing peptide chain. The order and specificity of the biosynthetic modules that form the NRPS protein template dictates the sequence and structure of the ultimate peptide products.

Each module of an NRPS acts as a semi-autonomous active site containing discrete, folded protein domains responsible for catalyzing specific reactions required for peptide chain elongation. A minimal module (in a single module complex) consists of at least two core domains: 1) an adenylation domain responsible for activating an amino acid (or, occasionally, a hydroxy acid); and 2) a thiolation or acyl carrier domain responsible for transferring activated intermediates to an enzyme-bound pantetheine cofactor. Most modules also contain 3) a condensation domain responsible for catalyzing peptide bond formation between activated intermediates. Supplementing these three core domains are a variable number of additional domains which can mediate, e.g., N-methylation (M or methylation domain) and L- to D-conversion (E or epimerization domain) of a bound amino acid intermediate, and heterocyclic ring formation (Cy or cyclization domain). The domains are usually characterized by specific amino acid motifs or features. It is the combination of such auxiliary domains acting locally on tethered intermediates within nearby modules that contributes to the enormous structural and functional diversity of the mature peptide products assembled by NRPS and mixed NRPS/PKS enzyme complexes.

The adenylation domain of each minimal module catalyzes the specific recognition and activation of a cognate amino acid. In this early step of non-ribosomal peptide biosynthesis, the cognate amino acid of each NRPS module is bound to the adenylation domain and activated as an unstable acyl adenylate (with concomitant ATP-hydrolysis). See, e.g., Stachelhaus et al., 1999, Chem. Biol. 6: 493-505 and Challis et al., 2000, Chem. Biol. 7: 211-224, each incorporated herein by reference in its entirety. In most NRPS modules, the acyl adenylate intermediate is next transferred to the T (thiolation) domain (also referred to as a peptidyl carrier protein or PCP domain) of the module where it is converted to a thioester intermediate and tethered via a transthiolation reaction to a covalently bound enzyme cofactor (4′-phosphopantetheinyl (4′-PP) intermediate). Modules responsible for incorporating D-configured or N-methylated amino acids may have extra modifying domains which, in several NRPSs studied, are located between the A and T domains.

The enzyme-bound intermediates in each module are then assembled into the peptide product by stepwise condensation reactions involving transfer of the thioester-activated carboxyl group of one residue in one module to, e.g., the adjacent amino group of the next amino acid in the next module while the intermediates remain linked covalently to the NRPS. Each condensation reaction is catalyzed by a condensation domain which is usually positioned between two minimal modules. The number of condensation domains in a NRPS generally corresponds to the number of peptide bonds present in the final (linear) peptide. An extra C domain has been found in several NRPSs (e.g., at the amino terminus of cyclosporin synthetase and the carboxyl terminus of rapamycin; see, e.g., Konz and Marahiel, supra) that has been proposed to be involved in peptide chain termination and cyclization reactions. Many other NRPS complexes, however, release the fall length chain in a reaction catalyzed by a C-terminal thioesterase (Te) domain (of approximately 28K-35K relative molecular weight).

Thioesterase domains of most NRPS complexes use a catalytic triad (similar to that of the well-known chymotrypsin mechanism) which includes a conserved serine (less often a cysteine or aspartate) residue in a conserved three-dimensional configuration relative to a histidine and an acidic residue. See, e.g. V. De Crecy-Lagard in “Comprehensive Natural Products Chemistry”, Volume 4, ed. by J. W. Kelly, Elsevier, N.Y., 1999, pp. 221-238, each incorporated herein by reference in its entirety. Thioester cleavage is a two step process. In the first (acylation) step, the full length peptide chain is transferred from the thiol tethered enzyme intermediate in the thiolation domain (see above) to the conserved serine residue in the Te domain, forming an acyl-O—Te ester intermediate. In the second (deacylation) step, the Te domain serine ester intermediate is either hydrolyzed (thereby releasing a linear, full length product) or undergoes cyclization, depending on whether the ester intermediate is attacked by water (hydrolysis) or by an activated intramolecular nucleophile (cyclization).

Sequence comparisons of C-terminal thioesterase domains from diverse members of the NRPS superfamily have revealed a conserved motif comprising the serine catalytic residue (GXSXG motif), often followed by an aspartic acid residue about 25 amino acids downstream from the conserved serine residue. A second type of thioesterase, a free thioesterase enzyme, is known to participate in the biosynthesis of some peptide and polyketide secondary metabolites. See e.g., Schneider and Marahiel, 1998, Arch. Microbiol. 169: 404-410, and Butler et al., 1999, Chem. Biol. 6: 87-292, each incorporated herein by reference in its entirety. These thioesterases are often required for efficient natural product synthesis (See United States Patent Application Number 20020192773). Butler et al. have postulated that the free thioesterase found in the polyketide tylosin gene cluster—which is required for efficient tylosin production—may be involved in editing and proofreading functions.

The modular organization of the NRPS multienzyme complex is mirrored at the level of the genomic DNA encoding the modules. The organization and DNA sequences of the genes encoding several different NRPSs have been studied. (See, e.g., Marahiel, 1997, Chem. Biol. 4: 561-567, incorporated herein by reference in its entirety). Conserved sequences characterizing particular NRPS functional domains have been identified by comparing NRPS sequences derived from many diverse organisms and those conserved sequence motifs have been used to design probes useful for identifying and isolating new NRPS genes and modules.

The modular structures of PKS and NRPS enzyme complexes can be exploited to engineer novel enzymes having new specificities by changing the numbers and positions of the modules at the DNA level by genetic engineering and recombination in vivo. Functional hybrid NRPSs have been constructed, for example, based on whole-module fusions. See, e.g., Gokhale et al., 1999, Science 284: 482-485; Mootz et al., 2000, Proc. Natl. Acad. Sci. U.S.A. 97: 5848-5853, incorporated herein by reference in their entirety. Recombinant techniques may be used to successfully swap domains originating from a heterologous PKS or NRPS complex. See, e.g., Schneider et al., 1998, Mol. Gen. Genet. 257: 308-318; McDaniel et al., 1999, Proc. Natl. Acad. Sci. U.S.A. 96: 1846-1851; U.S. Pat. Nos. 5,652,116 and 5,795,738; and International Patent Number WO 00/56896; incorporated herein by reference in their entirety.

Engineering a new substrate specificity within a module, by altering residues which form the substrate binding pocket of the adenylation domain, has also been described. See, e.g., Cane and Walsh, 1999, Chem. Biol. 6: 319-325; Stachelhaus et al., 1999, Chem. Biol. 6: 493-505; and International Patent Application Number WO 00/52152; each incorporated herein by reference in its entirety. By comparing the sequence of the B. subtilis peptide synthetase GrsA adenylation domain (PheA, whose structure is known) with sequences of 160 other adenylation domains from pro- and eukaryotic NRPSs, for example, Stachelhaus et al. (supra) and Challis et al., 2000, Chem. Biol. 7: 211-224 defined adenylation (A) domain signature sequences (analogous to codons of the genetic code) for a variety of amino acid substrates. From the collection of those signature sequences, a putative NRPS selectivity-conferring code (with degeneracies like the genetic code) was formulated.

The ability to engineer NRPSs having new modular template structures and new substrate specificities by adding, deleting or exchanging modules (or by adding, deleting or exchanging domains within one or more modules) will enable the production of novel peptides having altered and potentially advantageous properties. A combinatorial library comprising over 50 novel polyketides, for example, was prepared by systematically modifying the PKS that synthesizes an erythromycin precursor (DEBS) by substituting counterpart sequences from the rapamycin PKS (which encodes alternative substrate specificities). See, e.g., International Patent Application NumberWO 00/63361 and McDaniel et al., 1999, supra, each incorporated herein by reference in its entirety.

Daptomycin is an example of a non-ribosomally synthesized peptide made by a NRPS (FIG. 1). Modification of the genes encoding the proteins involved in the daptomycin biosynthetic pathway, including the daptomycin NRPS, provide a first step in producing modified Streptomyces roseosporus (NRRL 11379) as well as other host strains which can produce an improved antibiotic (for example, having greater potency); which can produce natural or new antibiotics in increased quantities; or which can produce other peptide products having useful biological properties. Compositions and methods relating to the Streptomyces roseosporus daptomycin biosynthetic gene cluster, including isolated nucleic acids and isolated proteins, are described in International Patent Application Number WO03/014297; hereby incorporated by reference.

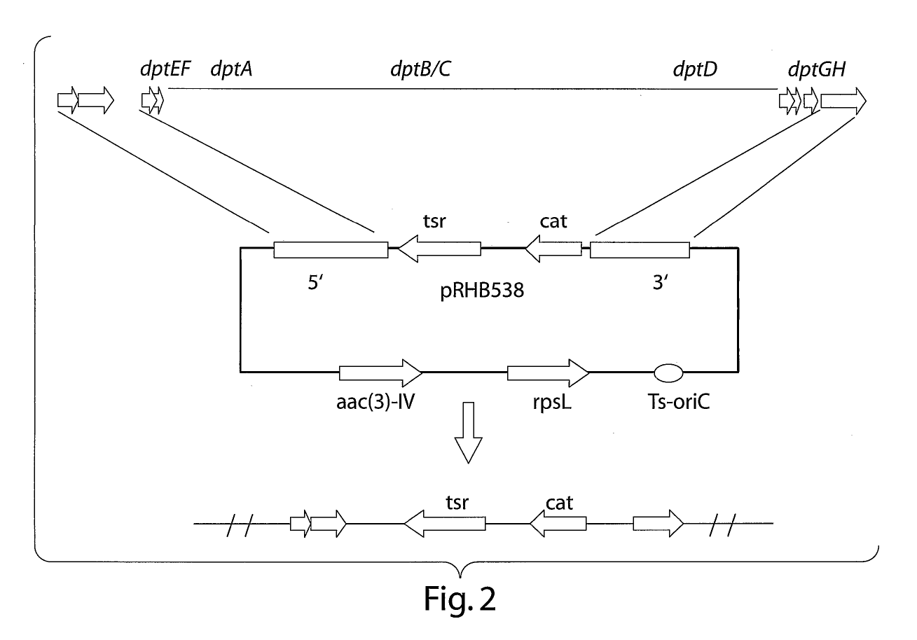

A54145 is another example of a non-ribosomally synthesized peptide made by a NRPS. A54145 is a cyclic lipopeptide antibiotic that is produced by the fermentation of Streptomyces fradiae (NRRL 18158). A54145 comprises a fatty acid chain linked via a three-amino acid chain to the N-terminal tryptophan of a cyclic 10-amino acid peptide (FIG. 2). The compound has similar in vitro anti-bactericidal activity to A21978C/daptomycin factors against various strains of S. aureus, S. epidermidis, Streptococcus pyogenes, and enterococci. Compositions and methods relating to the Streptomyces fradiae A54145 biosynthetic gene cluster, including isolated nucleic acids and isolated proteins, are described in International Patent Application Number WO03/060127; hereby incorporated by reference.

The genes encoding the proteins involved in the A54145 biosynthetic pathway, including the A54145 NRPS, provide a first step in producing modified Streptomyces fradiae as well as other host strains which can produce an improved antibiotic (for example, having greater potency); which can produce natural or new antibiotics in increased quantities; or which can produce other peptide products having useful biological properties.

Methods of Altering Gene Clusters for Production of Novel Compounds by NRPS

Alteration of NRPS Polypeptide Modules and Domains

In one aspect, the invention provides a method of altering the number or position of the modules in an NRPS to obtain the compounds of Formula I or compounds of any of Formula F1-F22. In one embodiment, one or more domains may be deleted from the NRPS. In this case, the product produced by the NRPS will have a chemical change relative to the peptide produced in the absence of the deletion, e.g., if an epimerization and/or methylation domain is deleted.

In another embodiment, one or more domains may be added to the NRPS. In this case, the peptide synthesized by the NRPS will have an additional chemical change. For instance, if an epimerization domain or a methylation domain is added, the resultant peptide will contain an extra D-amino acid or will contain a methylated amino acid, respectively. In a yet further embodiment, one or more modules may be mutated, e.g., an adenylation domain may be mutated such that it has a different amino acid specificity than the naturally-occurring adenylation domain. With the amino acid code in hand, one of skill in the art can perform mutagenesis, by a variety of well known techniques, to exchange the code in one module for another code, thus altering the ultimate amino acid composition and/or sequence of the resulting peptide synthesized by the altered NRPS. In another embodiment, one or more subunits may be added or deleted to the NRPS.

In a still further embodiment, one or more domains, modules or subunits may be substituted with another domain, module or subunit in order to produce novel peptides by complementation (See International Patent Application Number WO 01/30985, providing, inter alia, methods for substituting modules). In this case, the peptide produced by the altered NRPS will have, e.g., one or more different amino acids compared to the naturally-occurring peptide. In addition, different combinations of insertions, deletions, substitutions and mutations of domains, modules or subunits may be used to produce a peptide of interest. For instance, one may substitute a modified module, domain or subunit for a naturally-occurring one, or may substitute a naturally-occurring module, domain or subunit from the NRPS from one organism for a module, domain or subunit of an NRPS from another organism. Modifications of the modules, domains and subunits may be performed by site-directed mutagenesis, domain exchange (for module or subunit modification), deletion, insertion or substitution of a domain in a module or subunit, or deletion, insertion or substitution of a module in a subunit. Further, a domain, module or subunit may be disrupted such that it does not function using any method known in the art. These disruptions include, e.g., such techniques as a single crossover disruptant or replacement through homologous recombination by another gene (e.g., a gene that permits selection or screening).

The products produced by the modified NRPS complexes will have different incorporated amino acids, different chemical alterations of the amino acids (e.g., methylation and epimerization). The domains, modules or subunits may be derived from any number of NRPS desired, including two, three or four NRPS. Further, the invention contemplates these altered NRPS complexes with and without an integral thioesterase domain.

The source of the modules, domains and/or subunits may be derived from the daptomycin biosynthetic gene cluster NRPS, the A54145 biosynthetic gene cluster NRPS, or may be derived from any NRPS that encodes another lipopeptide or other peptide source. These peptide sources include glycopeptide gene clusters, mixed pathway gene clusters and siderophore gene clusters. Artificial NRPSs and methods for making them, have been described in International Patent Application Number WO01/30985, herein incorporated by reference. Further, the source of the modules, domains and/or subunits may be obtained from any appropriate source, including both streptomycete and non-streptomycete sources. Non-streptomycete sources include actinomycetes, e.g., Amycolatopsis; prokaryotic non-actinomycetes, e.g., Bacillus and cyanobacteria; and non-bacterial sources, e.g., fungi.

An NRPS or portion thereof may be heterologous to a host cell of interest or may be endogenous to the host cell. In one embodiment, the NRPS or a portion thereof (e.g., a domain, module or subunit thereof) is introduced into the host cell on any vector known to one having ordinary skill in the art, e.g., a plasmid, a cosmid, bacteriophage or BAC. The host cell into which the NRPS or portion thereof is introduced may contain an endogenous NRPS or portion thereof (e.g., a domain, module or subunit thereof). Alternatively, a heterologous NRPS or portion thereof may be introduced into the host cell containing the heterologous NRPS or portion thereof. The first NRPS, or another NRPS, or domain, module or subunit of an NRPS may have either a naturally-occurring sequence or a modified sequence. In another embodiment, the NRPS or portion thereof is endogenous to the host cell, e.g., the host cell is S. fradiae in the case of A54145 or is S. roseosporus in the case of daptomycin. A naturally-occurring or modified NRPS, or a domain, module or subunit thereof may be introduced into the host cell comprising the endogenous NRPS or portion thereof. The heterologous domains, modules, subunits or NRPS may comprise a constitutive or regulatable promoter, which are known to those having ordinary skill in the art. The promoter can be either homologous or heterologous to the nucleic acid molecule being introduced into the cell. In certain embodiments, the promoter may be from the A54145 biosynthetic gene cluster or the daptomycin biosynthetic gene cluster, as described above.

The nucleic acid molecule comprising the NRPS or portion thereof (e.g., a domain, module or subunit) may be maintained episomally or integrated into the genome. The nucleic acid molecule may be introduced into the genome at, e.g., phage integration sites. Further, the nucleic acid molecule may be introduced into the genome at the site of an endogenous or heterologous NRPS or portion thereof or elsewhere in the genome. The nucleic acid molecule may be introduced in such a way to disrupt all or part of the function of a domain, module or subunit of an NRPS already present in the genome, or may be introduced in a manner that does not disturb the function of the NRPS or portion thereof.

The peptides produced by these NRPSs may be useful as new compounds or may be useful in producing new compounds. In a preferred embodiment, the new compounds are useful as or may be used to produce antibiotic compounds. In another preferred embodiment, the new compounds are useful as or may be used to produce other peptides having useful activities, including but not limited to antibiotic, antifungal, antiviral, antiparasitic, antimitotic, cytostatic, antitumor, immuno-modulatory, anti-cholesterolemic, siderophore, agrochemical (e.g., insecticidal) or physicochemical (e.g., surfactant) properties.

Further diversity of non-ribosomally synthesized peptides and polyketides may also be achieved by expressing one or more NRPS and PKS genes (encoding natural, hybrid or otherwise altered modules or domains) in heterologous host cells, i.e., in host cells other than those from which the NRPS and PKS genes or modules originated.

### 3. Post Peptide Modification

The compounds of the present invention may be obtained by first assembling the core of the molecule by any of the methods described above followed by synthetic manipulation of all or some of the remaining primary amino groups as described in U.S. Pat. Nos. 6,911,525; and 6,794,490 and in International Patent Application Numbers WO01/44272; WO01/44274; and WO01/44271.

Treatment of the primary amino group(s) with reagents such as isocyanates, isothiocyanates, activated esters, acid chlorides, sulfonylchlorides or activated sulfonamides, heterocycles bearing readily displaceable groups, imidates, lactones or reductively with aldehydes affords compounds in which one or more of substituents R¹, R^{aa1}, R^{aa2}, R⁶*, and R⁸** is independently monosubstituted amino, disubstituted amino, acylamino, ureido, guanidino, carbamoyl, sulfonamino, thioacylamino, thioureido, iminoamino, or phosphonamino.

In order to achieve these modifications, it may be necessary to protect certain functionalities in the molecule. Protecting these functionalities should be within the expertise of one skilled in the art following the disclosure of this invention. See, e.g., Greene, supra.

Cells and Methods for Making Cells that Can Express Recombinant NRPS

The present invention includes cells and methods for making cells that can express recombinant NRPS gene clusters that are capable of expressing the recombinant NRPS and capable of producing the various compounds of the invention. In certain specific embodiments, the cells are gram positive cells, including Streptomyces lividans, Streptomyces coelicolor, or Streptomyces roseosporus. In other specific embodiments of the invention, a recombinant NRPS is assembled from modules from a daptomycin or A54145 NRPS gene cluster. These genes may be “swapped” using recombination techniques known in the art or exemplified herein. In other embodiments, certain genes in the recombinant NRPS are deactivated or “knocked out” to avoid the expression product and its activity in the cell. [JILL, SHOULD WE MENTION 3MG HERE AND lptI?]

In a preferred embodiment, bacterial host cells are used to express the nucleic acid molecules of the instant invention. Useful expression vectors for bacterial hosts include bacterial plasmids, such as those from E. coli or Streptomyces, including pBluescript, pGEX-2T, pUC vectors, col E1, pCR1, pBR322, pMB9 and their derivatives, wider host range plasmids, such as RP4, phage DNAs, e.g., the numerous derivatives of phage lambda, e.g., NM989, ?GT10 and ?GT11, and other phages, e.g., M13 and filamentous single stranded phage DNA. A preferred vector is a bacterial artificial chromosome (BAC). A more preferred vector is pStreptoBAC, as described in Example 2 of International Patent Application Number 03/014297.

In other embodiments, eukaryotic host cells, such as yeast, insect or mammalian cells, may be used. Yeast vectors include Yeast Integrating plasmids (e.g., YIp5) and Yeast Replicating plasmids (the YRp and YEp series plasmids), Yeast centromere plasmids (the YCp series plasmids), pGPD-2, 2μ plasmids and derivatives thereof, and improved shuttle vectors such as those described in Gietz and Sugino, Gene, 74, pp. 527-34 (1988) (YIplac, YEplac and YCplac). Expression in mammalian cells can be achieved using a variety of plasmids, including pSV2, pBC12B1, and p91023, as well as lytic virus vectors (e.g., vaccinia virus, adeno virus, and baculovirus), episomal virus vectors (e.g., bovine papillomavirus), and retroviral vectors (e.g., murine retroviruses). Useful vectors for insect cells include baculoviral vectors and pVL 941.

Other aspects of the invention provide compounds and methods for making the compounds from recombinant cells described herein. The compounds can be produced by culturing the cells using techniques and conditions that are known in the art or described herein. The conditions for culturing the cells may include fermenting the cells with a lipopeptide tail precursor that promotes the production of a particular compound of the invention. This precursor may be taken up by the cell during fermentation and increase the production of a particular compound in the cell. A precursor provided to the cell during fermentation is sometimes called a fermentation feed and the resulting compound a feed product. The compounds of the invention produced by culturing or fermenting the cells of the invention may be further isolated from the fermentation product and/or purified.

### Preparation of Novel Depsipetides

### 1. Synthetic Processes

In order that this invention may be more fully understood, the following examples are set forth. These examples are for illustrative purposes only and are not to be construed as limiting the scope of the invention in any way.

### EXAMPLE 1-1

### Synthesis of Peptide Resin Compound 1

### Resin-Gly-Thr-Asp(OtBu)-DAsn(NHTrt)-Trp-NH₂ (1)

### Reaction 1

### Preparation of Resin-Gly-Thr-NHFmoc (2)

A solution of commercially available Nα-(9-Fluorenylmethoxycarbonyl)-L-threonine (2 mL of a 0.5 molar solution in N-methylpyrrolidine), 1,3-diisopropylcarbodiimide (2 mL of a 0.5 molar solution in N-methylpyrrolidine), and 1-hydroxy-benzotriazole (2 mL of a 0.5 molar solution in N-methylpyrrolidine) was added to commercially available glycine 2-chlorotrityl resin (334 mg). The mixture was shaken for one hour, filtered through a glass sinter funnel and a few beads were tested for the presence of a free amine using the standard Kaiser test (see E. Kaiser, et al., 1970, Anal. Biochem. 34: 595; and “Advanced Chemtech Handbook of Combinatorial, Organic and Peptide Chemistry” 2003-2004, page 208). The Kaiser test gave a blue color indicating that the reaction was incomplete therefore the coupling conditions above was repeated. After filtration through a glass sinter funnel the product bearing resin was washed with N-methylpyrrolidine (3×6 mL), methanol (3×6 mL), and again with N-methylpyrrolidine (3×6 mL) to give compound 2.

### Reaction 2

### Preparation of Resin-Gly-Thr-NH₂ (3)

Compound 2 was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then the solid was washed with N-methylpyrrolidine (3×6 mL), methanol (3×6 mL), and again with N-methylpyrrolidine (3×6 mL) to give compound 3.

### Reaction 3

### Preparation of Resin-Gly-Thr-Asp(OtBu)-NHFmoc (4)

Commercially available Nα-(9-Fluorenylmethoxycarbonyl)-L-aspartic acid β-tert-butyl ester (2 mL of a 0.5 molar solution in N-methylpyrrolidine), 1,3-diisopropylcarbodiimide (2 mL of a 0.5 molar solution in N-methylpyrrolidine), and 1-hydroxy-benzotriazole (2 mL of a 0.5 molar solution in N-methylpyrrolidine) were added to compound 3. The mixture was shaken for one hour, filtered through a glass sinter funnel and the coupling was repeated. The reaction mixture was filtered through a glass sinter funnel then the solid was washed with N-methylpyrrolidine (3×6 mL), methanol (3×6 mL), and again with N-methylpyrrolidine (3×6 mL) to give compound 4.

### Reaction 4

### Preparation of Resin-Gly-Thr-Asp(OtBu)-NH₂ (5)

Compound 4 was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then washed with N-methylpyrrolidine (3×6 mL), methanol (3×6 mL), and again with N-methylpyrrolidine (3×6 mL) to give compound 5.

### Reaction 5

### Preparation of Resin-Gly-Thr-Asp(OtBu)-DAsn(NHTrt)-NHFmoc (6)

A solution of commercially available Nα-(9-Fluorenylmethoxycarbonyl)-D-asparagine δ-N-trityl (2 mL of a 0.5 molar solution in N-methylpyrrolidine), 1,3-diisopropylcarbodiimide (2 mL of a 0.5 molar solution in N-methylpyrrolidine), and 1-hydroxy-benzotriazole (2 mL of a 0.5 molar solution in N-methylpyrrolidine) was added to resin 5. The reaction mixture was shaken for one hour, filtered through a glass sinter funnel and the coupling was repeated. The reaction mixture was filtered through a glass sinter funnel then the solid was washed with N-methylpyrrolidine (3×6 mL), methanol (3×6 mL), and again with N-methylpyrrolidine (3×6 mL) to give compound 6.

### Reaction 6

### Preparation of Resin-Gly-Thr-Asp(OtBu)-DAsn(NHTrt)-NH₂

Compound 6 was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then the solid was washed with N-methylpyrrolidine (3×6 mL), methanol (3×6 mL), and again with N-methylpyrrolidine (3×6 mL) to give compound 7.

Reaction 7

### Preparation of Resin-Gly-Thr-Asp(OtBu)-DAsn(NHTrt)-Trp-NHFmoc (8)

Commercially available Nα-(9-Fluorenylmethoxycarbonyl)-L-tryptophan (2 mL of a 0.5 molar solution in N-methylpyrrolidine), 1,3-diisopropylcarbodiimide (2 mL of a 0.5 molar solution in N-methylpyrrolidine), and 1-hydroxy-benzotriazole (2 mL of a 0.5 molar solution in N-methylpyrrolidine) were added to resin 7. The reaction mixture was shaken for one hour, then filtered through a glass sinter funnel and the coupling was repeated. The reaction mixture was filtered through a glass sinter funnel then the solid was washed with N-methylpyrrolidine (3×6 mL), methanol (3×6 mL), and again with N-methylpyrrolidine (3×6 mL) to give compound 8.

### Reaction 8

### Preparation of Resin-Gly-Thr-Asp(OtBu)-DAsn(NHTrt)-Trp-NH₂ (1)

Compound 8 was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then the solid was washed with N-methylpyrrolidine (3×6 mL), methanol (3×6 mL), and again with N-methylpyrrolidine (3×6 mL) to give resin peptide compound 1.

### EXAMPLE 1-2

### Synthesis of Peptide Resin Compound 9

### Resin-Glu(αOAllyl)-DSer(OtBu)-Gly-Asp(OtBu)-DAla-Asp-Orn(HNBoc)-NH₂ (9)

### Reaction 1

### Preparation of Resin-Glu(αOAllyl)-NHFmoc (10)

To a suspension of commercially available 4-hydroxymethylphenoxy resin (Wang resin, 5 g, 0.4 mmol/g) in dichloromethane (60 mL) was added 1,3-diisopropylcarbodiimide (0.940 mL), 4-dimethylaminopyridine (24 mg in N-methylpyrrolidine (1 mL)), and commercially available Nα-(9-Fluorenylmethoxycarbonyl)-L-glutamic acid α-allyl ester (2.46 g in N-methylpyrrolidine (9 mL)). The reaction mixture was stirred for 16 hours, filtered through a glass sinter funnel, and the solid was washed with N-methylpyrrolidine and dichloromethane and dried under reduced pressure to give compound 10.

### Reaction 2

### Preparation of Resin-Glu(αOAllyl)-NH₂ (11)

Compound 10 (526 mg) was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then the solid was washed with N-methylpyrrolidine (3×6 mL), methanol (3×6 mL), and again with N-methylpyrrolidine (3×6 mL) to give compound 11.

### Reaction 3

### Preparation of Resin-Glu(αOAllyl)-DSer(OtBu)-NHFmoc (12)

Commercially available Nα-(9-Fluorenylmethoxycarbonyl)-D-serine-tert-butyl ether (2 mL of a 0.5 molar solution in N-methylpyrrolidine), 1,3-diisopropylcarbodiimide (2 mL of a 0.5 molar solution in N-methylpyrrolidine), and 1-hydroxy-benzotriazole (2 mL of a 0.5 molar solution in N-methylpyrrolidine) were added to resin 11. The reaction mixture was shaken for one hour, then filtered through a glass sinter funnel and the coupling was repeated. The reaction mixture was filtered through a glass sinter funnel then the solid was washed with N-methylpyrrolidine (3×6 mL), methanol (3×6 mL), and again with N-methylpyrrolidine (3×6 mL) to give compound 12.

### Reaction 4

### Preparation of Resin-Glu(αOAllyl)-DSer(OtBu)-NH— (13)

Compound 12 was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then the solid was washed with N-methylpyrrolidine (3×6 mL), methanol (3×6 mL), and again with N-methylpyrrolidine (3×6 mL) to give compound 13.

### Reaction 5

### Preparation of Resin-Glu(αOAllyl)-DSer(OtBu)-Gly-NHFmoc (14)

Commercially available Nα-(9-Fluorenylmethoxycarbonyl)-L-glycine (2 mL of a 0.5 molar solution in N-methylpyrrolidine), 1,3-diisopropylcarbodiimide (2 mL of a 0.5 molar solution in N-methylpyrrolidine), and 1-hydroxy-benzotriazole (2 mL of a 0.5 molar solution in N-methylpyrrolidine) were added to resin 13. The reaction mixture was shaken for one hour, then filtered through a glass sinter funnel and the coupling was repeated. The reaction mixture was filtered through a glass sinter funnel then the solid was washed with N-methylpyrrolidine (3×6 mL), methanol (3×6 mL), and again with N-methylpyrrolidine (3×6 mL) to give compound 14.

### Reaction 6

### Preparation of Resin-Glu(αOAllyl)-DSer(OtBu)-Gly-NH₂ (15)

Compound 14 was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then the solid was washed with N-methylpyrrolidine (3×6 mL), methanol (3×6 mL), and again with N-methylpyrrolidine (3×6 mL) to give compound 15.

### Reaction 7

### Preparation of Resin-Glu(αOAllyl)-DSer(OtBu)-Gly-Asp(OtBu)-NHFmoc (16)

Commercially available Nα-(9-Fluorenylmethoxycarbonyl)-L-aspartic acid β-tert-butyl ester (2 mL of a 0.5 molar solution in N-methylpyrrolidine), 1,3-diisopropylcarbodiimide (2 mL of a 0.5 molar solution in N-methylpyrrolidine), and 1-hydroxy-benzotriazole (2 mL of a 0.5 molar solution in N-methylpyrrolidine) were added to resin 15. The reaction mixture was shaken for one hour, through a glass sinter tunnel and the coupling was repeated. The reaction mixture was filtered through a glass sinter funnel then the solid was washed with N-methylpyrrolidine (3×6 mL), methanol (3×6 mL), and again with N-methylpyrrolidine (3×6 mL) to give compound 16.

### Reaction 8

### Preparation of Resin-Glu(αOAllyl)-DSer(OtBu)-Gly-Asp(OtBu)-NH₂ (17)

Compound 16 was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then the solid was washed with N-methylpyrrolidine (3×6 mL), methanol (3×6 mL), and again with N-methylpyrrolidine (3×6 mL) to give compound 17.

### Reaction 9

### Preparation of Resin-Glu(αOAllyl)-DSer(OtBu)-Gly-Asp(OtBu)-DAla-NHFmoc (18)

A solution of commercially available Nα-(9-Fluorenylmethoxycarbonyl)-D-alanine ((2 mL of a 0.5 molar solution in N-methylpyrrolidine), 1,3-diisopropylcarbodiimide (2 mL of a 0.5 molar solution in N-methylpyrrolidine), and 1-hydroxy-benzotriazole (2 mL of a 0.5 molar solution in N-methylpyrrolidine) was added to resin 17. The reaction mixture was shaken for one hour, filtered through a glass sinter funnel and the coupling was repeated. The reaction mixture was filtered through a glass sinter funnel then the solid was washed with N-methylpyrrolidine (3×6 mL), methanol (3×6 mL), and again with N-methylpyrrolidine (3×6 mL) to give compound 18.

### Reaction 10

### Preparation of Resin-Glu((αOAllyl)-DSer(OtBu)-Gly-Asp(OtBu)-DAla-NH₂ (19)

Compound 18 was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then the solid was washed with N-methylpyrrolidine (3×6 mL), methanol (3×6 mL), and again with N-methylpyrrolidine (3×6 mL) to give compound 19.

### Reaction 11

### Preparation of Glu(αOAllyl)-DSer(OtBu)-Gly-Asp(OtBu)-DAla-Asp(OtBu)-NHFmoc (20)

Commercially available Nα-(9-Fluorenylmethoxycarbonyl)-L-aspartic acid β-tertbutyl ester ((2 mL of a 0.5 molar solution in N-methylpyrrolidine), 1,3-diisopropylcarbodiimide (2 mL of a 0.5 molar solution in N-methylpyrrolidine), and 1-hydroxy-benzotriazole (2 mL of a 0.5 molar solution in N-methylpyrrolidine) was added to resin 19. The reaction mixture was shaken for one hour, filtered through a glass sinter funnel and the coupling was repeated. The reaction mixture was filtered through a glass sinter funnel then the solid was washed with N-methylpyrrolidine (3×6 mL), methanol (3×6 mL), and again with N-methylpyrrolidine (3×6 mL) to give compound 20.

### Reaction 12

### Preparation of Resin-Glu(αOAllyl)-DSer(OtBu)-Gly-Asp(OtBu)-DAla-Asp(OtBu)-NH₂ (21)

Compound 20 was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then the solid was washed with N-methylpyrrolidine (3×6 μL), methanol (3×6 mL), and again with N-methylpyrrolidine (3×6 mL) to give compound 21.

### Reaction 13

### Preparation of Resin-Glu(αOAllyl)-DSer(OtBu)-Gly-Asp(OtBu)-DAla-Asp(OtBu)-Orn-NHFmoc (22)

A solution of commercially available Nα-(9-Fluorenylmethoxycarbonyl)-N-δ-(tertbutoxycarbonyl)-L-ornithine (2 mL of a 0.5 molar solution in N-methylpyrrolidine), 1,3-diisopropylcarbodiimide (2 mL of a 0.5 molar solution in N-methylpyrrolidine), and 1-hydroxy-benzotriazole (2 mL of a 0.5 molar solution in N-methylpyrrolidine) was added to resin 21. The reaction mixture was shaken for one hour, then filtered through a glass sinter funnel and the coupling was repeated. The reaction mixture was filtered through a glass sinter funnel then the solid was washed with N-methylpyrrolidine (3×6 mL), methanol (3×6 mL), and again with N-methylpyrrolidine (3×6 mL) to give compound 22.

### Reaction 14

### Preparation of Resin-Glu(αOAllyl)-DSer(OtBu)-Gly-Asp(OtBu-DAla-AsM(OtBu)-Orn(NHBoc)-NH₂ (9)

Compound 22 was agitated in 20% piperidine in N-methylpyrrolidine (6 mL) for 30 minutes. The resin was filtered through a glass sinter funnel and re-suspended in 20% piperidine in N-methylpyrrolidine (6 mL) and agitated for 30 minutes. The reaction mixture was filtered through a glass sinter funnel then the solid was washed with N-methylpyrrolidine (3×6 mL), methanol (3×6 mL), and again with N-methylpyrrolidine (3×6 mL) to give compound 9.

### EXAMPLE 1-3

### Synthesis of Peptide Resin Compound 23

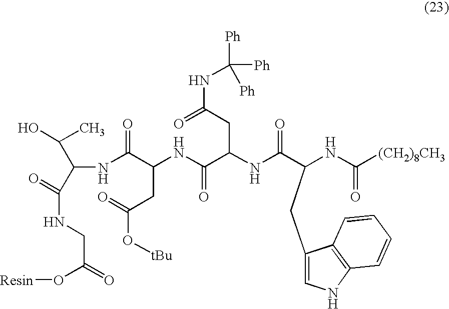

Reaction 1: Preparation of Compound 24

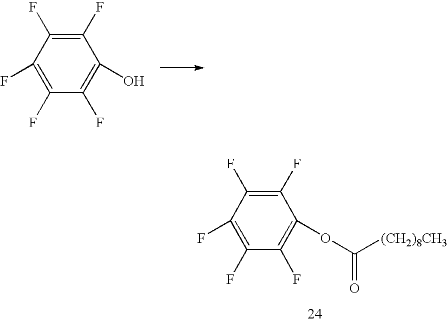

Pentafluorophenol (3.68 g) was dissolved in dichloromethane (40 mL) and cooled to 0° C. in an ice/NaCl bath. Decanoylchloride (4.15 mL) was added dropwise such that the temperature remained below 2° C. Once addition was complete, the reaction was stirred for an additional 2.5 hours at 0° C. The cooling bath was then removed and the reaction warmed to ambient temperature and stirred for 17 hours. The volatiles were removed under reduced pressure to give the crude product pentafluorophenyl ester 24, which could be used subsequently without further purification.

Reaction 2 Preparation of Compound 23

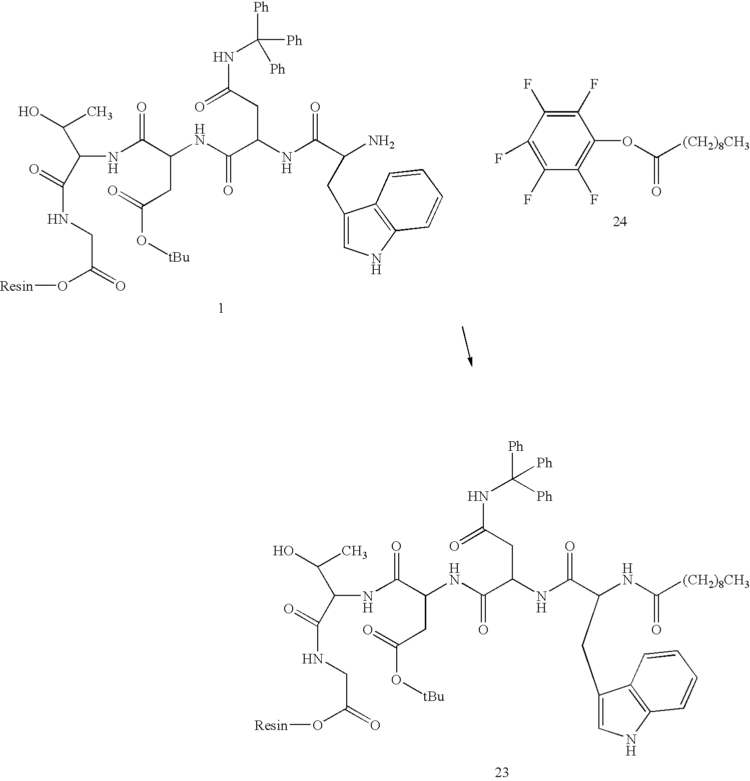

Resin peptide compound 1 (2 g) was added to a solution of the pentafluorophenyl ester of decanoic acid, 24, (440 mg) in dichloromethane. The mixture was shaken for 17 hours, filtered through a glass sinter funnel, and the reaction was judged to be incomplete using the Kaiser Test (vide supra). Decanoic acid (517 mg), 1-hydroxy-benzotriazole (446 mg), and 1,3-diisopropylcarbodiimide (438 μL) were dissolved in N-methylpyrrolidine (8 mL) and stirred for one hour. The resin was then added to the decanoic acid mixture then stirred for 8 hours, filtered through a glass sinter funnel and washed with N-methylpyrrolidine (3×6 mL), methanol (3×6 mL), and again with N-methylpyrrolidine (3×6 mL). The reaction was found to be complete using the Kaiser Test, yielding the resin bound lipopeptide 23.

### EXAMPLE 1-4

### Synthesis of Compound C352

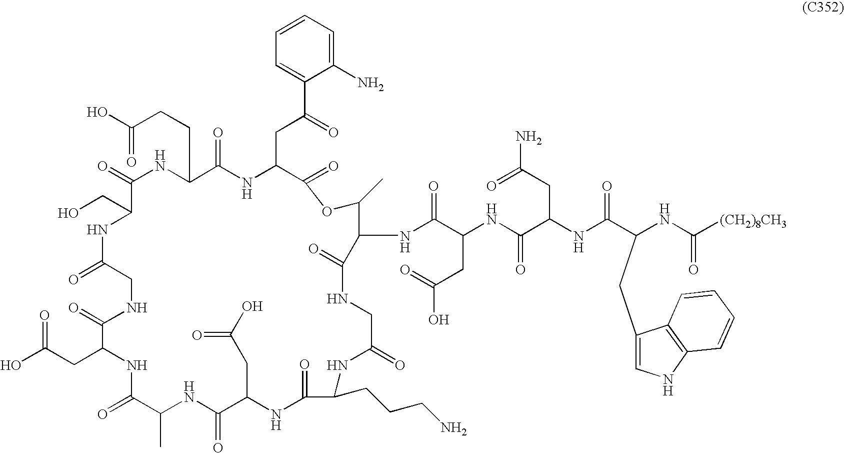

### Reaction 1

### Preparation of Compound (25)

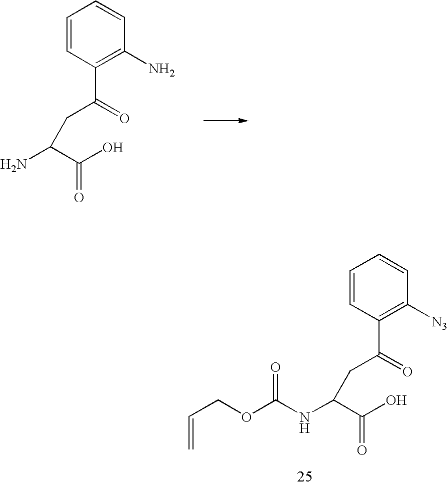

Commercially available Kynurenine (3 g) was suspended in acetonitrile (100 mL) and water (30 mL). Diisopropylethylamine (DIPEA, 5.0mL) was added dropwise to the solution and stirring was continued until the solution was homogeneous. The solution was then cooled to 0° C. in an ice/sodium chloride bath and a solution of allyloxycarbonyl oxysuccinimide (AllocOSu, 4.3 g) in acetonitrile (30 mL) was added. The reaction mixture was stirred for 3 hours then concentrated to remove acetonitrile, basified with 5% K₂CO₃ solution (220 mL) and washed with ethyl acetate (5×90 mL) and dichloromethane (1×90 mL). The aqueous portion was then acidified to pH 1 and extracted with ethyl acetate (4×90 mL). Combined acidic organic washes were dried with anhydrous MgSO₄ and evaporated to give crude product (4.85 g). Purification by column chromatography on silica gel, eluting with dichloromethane methanol 19:1, gave the desired intermediate, L-2-N-(allyloxycarbonyl)-4-(2-aminophenyl)-4-oxobutanoic acid, after evaporation of the solvent as a yellow solid 2.92 g. This solid (2.9 g) was dissolved in 4N HCl (100 mL) and cooled to 0° C. in an ice/sodium chloride bath. A solution of NaNO₂ (0.76 g) in water (10 mL) was added dropwise such that the temperature remained below 3° C., and the resultant solution was stirred for 2.5 hours at 0° C. A solution of NaN₃ (1.95 g) in water (10 mL) was added dropwise such that the temperature remained below 3° C. and the resultant solution was warmed to ambient temperature and stirred over 19 hours. The reaction mixture was poured into water (250 mL) and extracted with dichloromethane (4×100 ml). The combined organic washes were dried with anhydrous MgSO₄ and evaporated to the desired product compound 25 (2.86 g).

### Reaction 2

### Preparation of Compound (26)

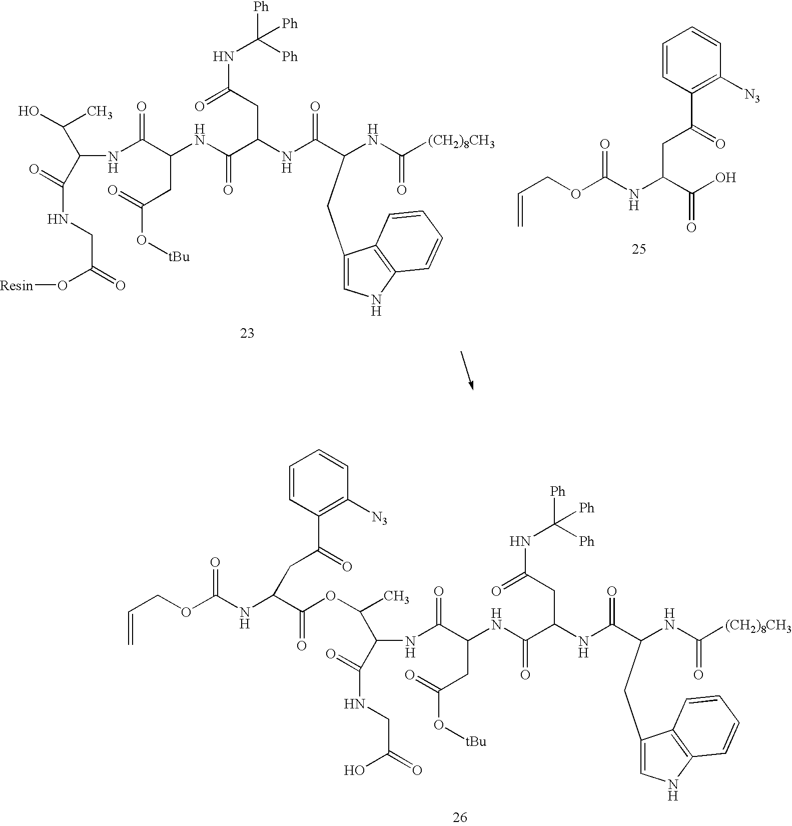

L-2-N-(allyloxycarbonyl)-4-(2-azidophenyl)-4-oxobutanoic acid 25 (636 mg), 4-dimethylaminopyridine (25 mg), and N-methyl-2-chloropyridinium iodide (511 mg) were flushed well with argon, then suspended in dichloromethane (10 mL). Triethylamine (560 μL) was added and the reaction mixture was stirred to give a homogeneous solution. Resin lipopeptide 23 (667 mg) was added to the solution and the flask was flushed again with argon and shaken for 17 hours. A 20 mg sample of the resin was removed to test the reaction for completion (20 mg of resin in dichloromethane (0.6 mL) was treated with 2,2,2-trifluoroethanol, (0.2 mL) and acetic acid (0.2 mL) and stirred for 3 hours. The reaction mixture was filtered through a glass sinter funnel, and the solvent was evaporated to give a residue. Liquid Chromatography/Mass Spectral analysis of the residue indicated the reaction was incomplete). Coupling was judged to be incomplete so the resin was dried under reduced pressure for 5 days, and the above coupling was repeated over another 17 hours. The reaction mixture was filtered through a glass sinter funnel and the solid was washed well with dichloromethane. The solid was then suspended in dichloromethane (6 mL), 2,2,2-trifluoroethanol (2 mL), acetic acid (2 mL), and shaken for 5 hours. The reaction mixture was filtered through a glass sinter funnel and evaporation of the filtrate gave the crude desired peptide 26 (44 mg). The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were freeze-dried to give the pure product 26 (10.6 mg).

### Reaction 3

### Preparation of Compound (27)

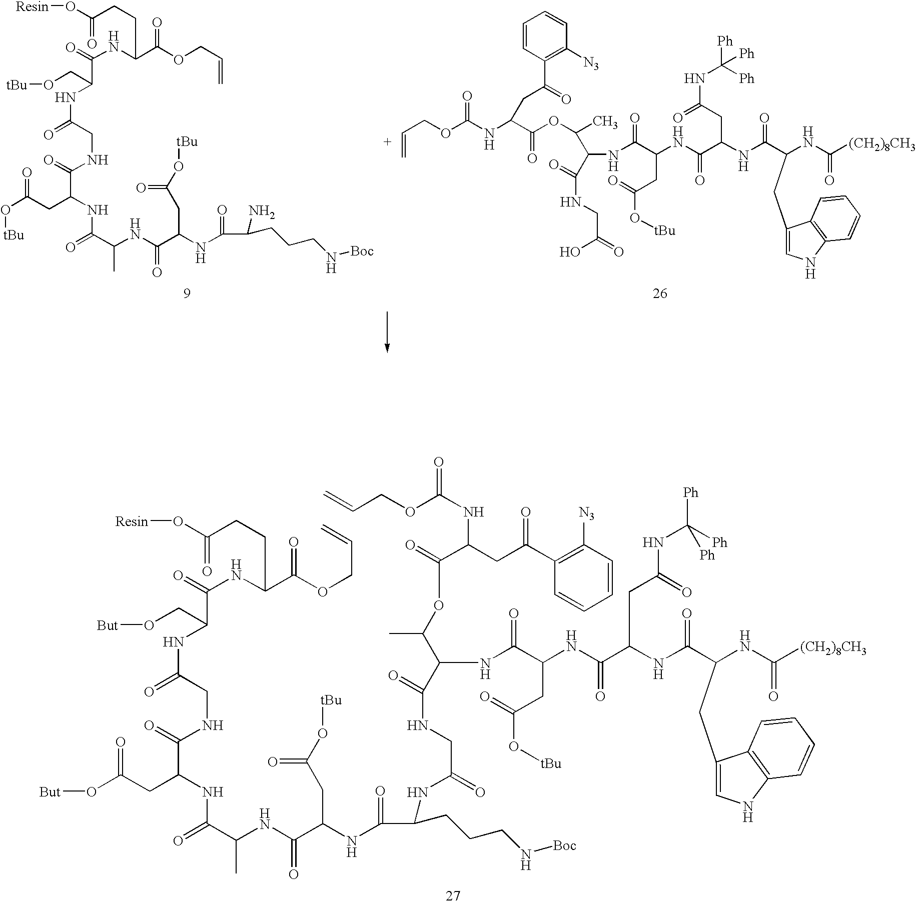

Hydroxy-benzotriazole (5 mg), 1,3-diisopropylcarbodiimide (6 μL), and peptide resin compound 9 (12.3 mg) were added to a solution of compound 26 (10.6 mg) in N-methylpyrrolidine (0.7 mL) then shaken for 22 hours. The resin was filtered through a glass sinter funnel and the coupling was judged to be complete using the Kaiser Test (vide supra), yielding resin bound lipopeptide 27.

### Reaction 4

### Preparation of Compound (28)

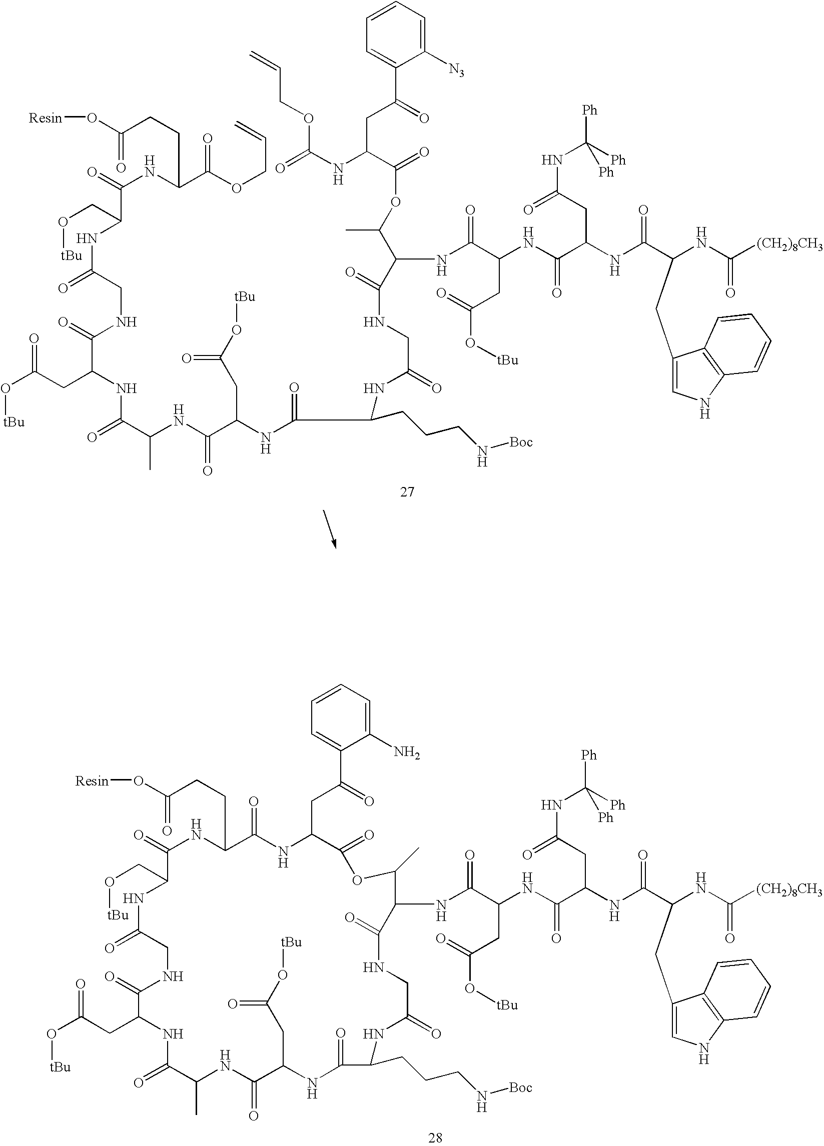

The dried resin 27 was placed under an argon atmosphere, and treated with a solution of tetrakis-(triphenylphosphine)palladium(0) (19 mg) in dichloromethane (1.47 mL), acetic acid (74 μL), and N-methylmorpholine (37 μL). The mixture was shaken for 4 hours at ambient temperature, filtered through a glass sinter tunnel, and the solid was washed with two times with N-methylmorpholine, two times with methanol, and again two times with N-methylmorpholine. 1-Hydroxy-benzotriazole (0.5 mL of a 0.5 molar solution in N-methylmorpholine) and 1,3-diisopropylcarbodiimide (0.5 mL of a 0.5 molar solution in N-methylmorpholine) were added to the resin. The reaction was shaken for 17 hours, filtered through a glass sinter funnel, and washed well with N-methylmorpholine to give the resin bound cyclized depsipeptide 28.

Reaction 5: Preparation of Compound (C352)

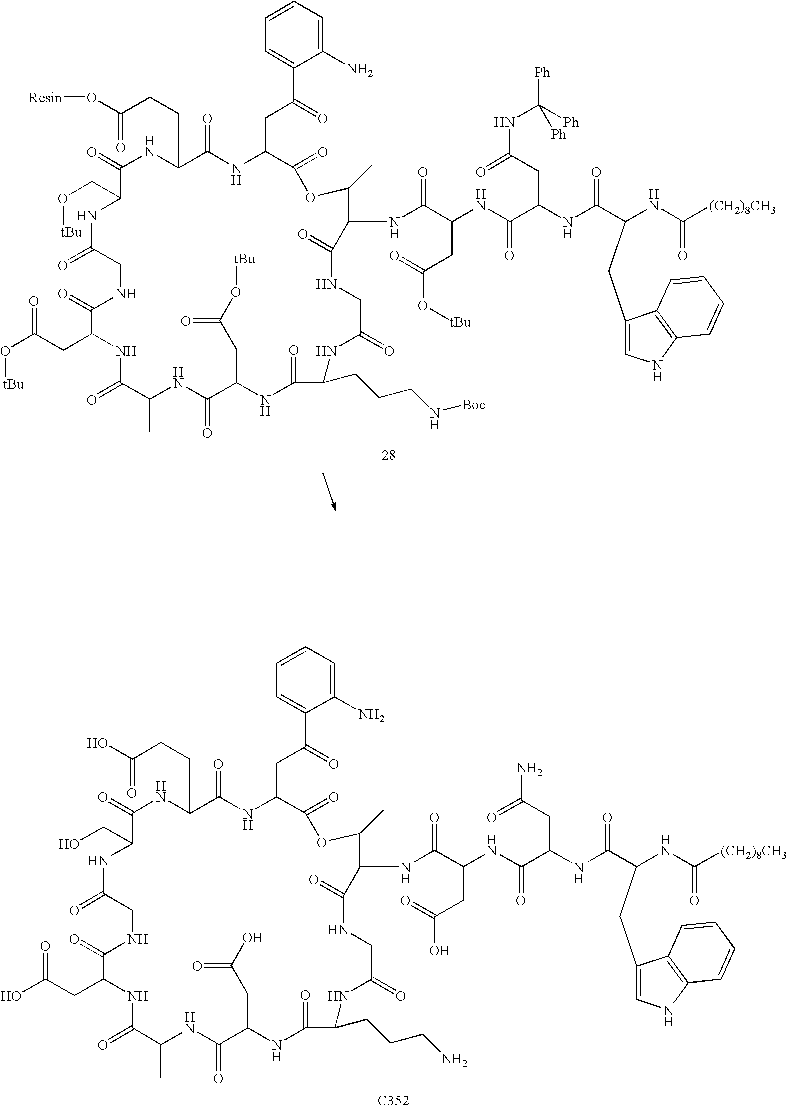

The dried resin 28 was suspended in dichloromethane, (4 mL) trifluoroacetic acid, (6 mL) ethanedithiol (250 μl), and triisopropylsilane (250 μl), and the reaction mixture was stirred for 3 hours at ambient temperature. The resin was filtered through a glass sinter funnel and the combined filtrates were evaporated under reduced pressure. Crude product was then partitioned between diethyl ether (6 mL), and water (3 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give the pure product C352(1.0 mg).

### EXAMPLE 1-5

### Synthesis of Compound C369

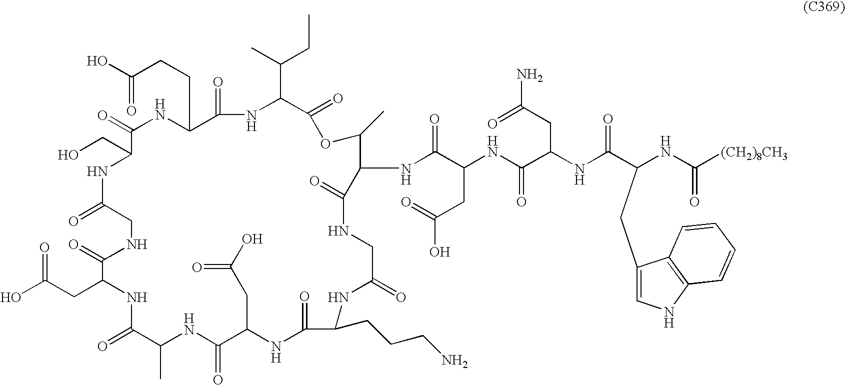

### Reaction 1

### Preparation of Compound (30

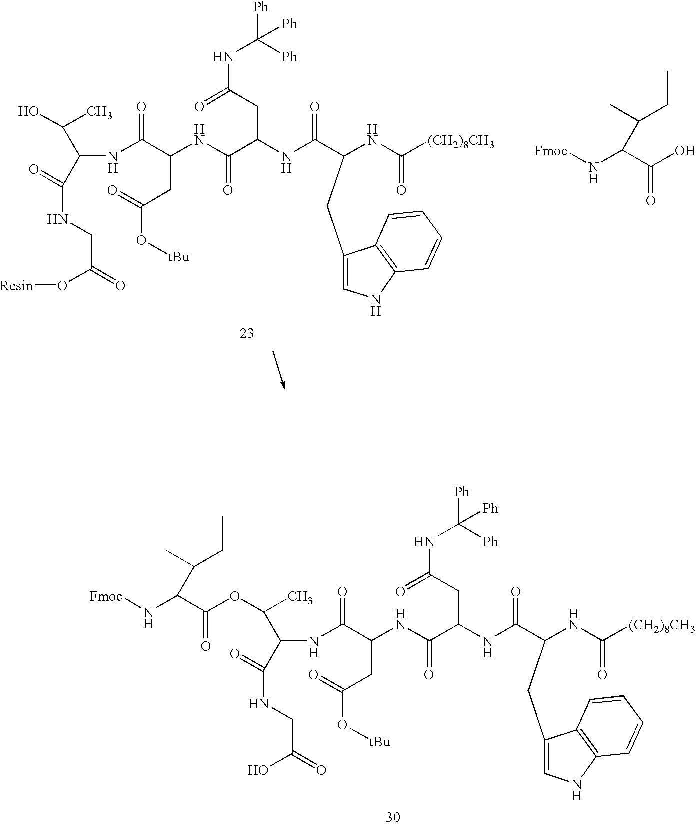 Compound 30 is obtained from compound 23 using either Method D or Method E (vide infra).
Method D

To the resin bound lipopeptide 23 (1 g) was added a solution of commercially available Nα-(9-Fluorenylmethoxycarbonyl)-L-isoleucine (618 mg), bromo-tris-pyrrolidinophosphonium hexafluorophosphate (PyBrOP, 815 mg), and Di-isopropylethylamine (914 μL), in dichloromethane (5 mL). Dimethylaminopyridine (5 mg) was added and the mixture was shaken for 2 hours. After 2 h, the mixture was filtered through a glass sinter funnel and washed with dichloromethane (3×10 mL) and the coupling procedure was repeated. The resulting resin was filtered through a glass sinter funnel, washed with dichloromethane (3×10 mL) and methanol (3×10 mL), and dried under diminished pressure over potassium hydroxide pellets. This dried resin was suspended in dichloromethane (3 mL), 2,2,2-trifluoroethanol (1 mL), and acetic acid (1 mL), and shaken for 3 hours. The resin was filtered through a glass sinter funnel and evaporation of the filtrate gave the desired peptide 30 (400 mg) as a white solid.

Method E

Commercially available Nα-(9-Fluorenylmethoxycarbonyl)-L-isoleucine (95 mg), 4-dimethylaminopyridine (6 mg), and N-methyl-2-chloropyridinium iodide (69 mg) were flushed well with argon then suspended in dichloromethane (2.7 mL). Triethylamine (76 μL) was added and the reaction mixture was stirred to give a homogeneous solution. Resin lipopeptide 23 (200 mg) was added to the solution, the flask was flushed again with argon and then the reaction mixture was shaken for 14 hours. The resulting resin was then filtered through a glass sinter funnel and washed well with dichloromethane. The solid was suspended in dichloromethane (6 mL), 2,2,2-trifluoroethanol (2 mL), and acetic acid (2 mL), and shaken for 3 hours. The resin was filtered through a glass sinter funnel and evaporation of the filtrate gave the desired peptide 30 (54 mg) as a white solid.

### Reaction 2

### Preparation of Compound (31)

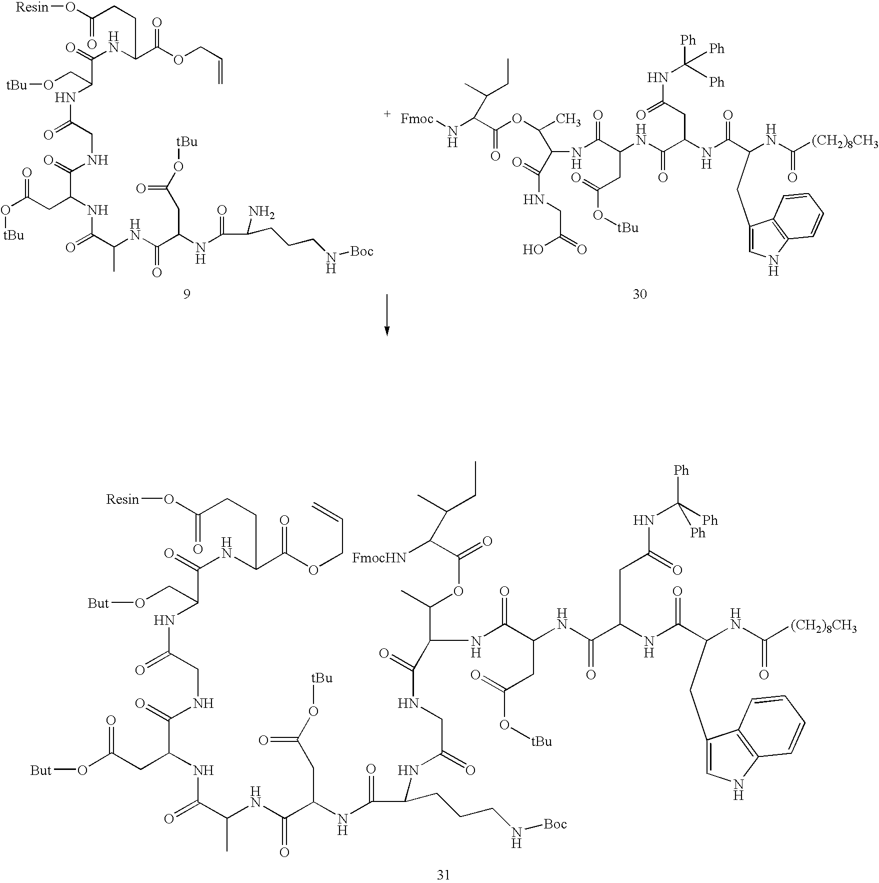

1-Hydroxy-benzotriazole (26 mg), 1,3-diisopropylcarbodiimide (30 μL), and peptide resin compound 9 (64 mg) were added to a solution of the depsipeptide 30 (54 mg) in N-methylmorpholine (3.8 mL), and the resulting mixture was shaken for 22 hours. The resin was filtered through a glass sinter funnel, and the coupling was judged to be complete using the Kaiser Test (vide supra), yielding the resin bound depsipeptide 31.

### Reaction 3

### Preparation of Compound (32)

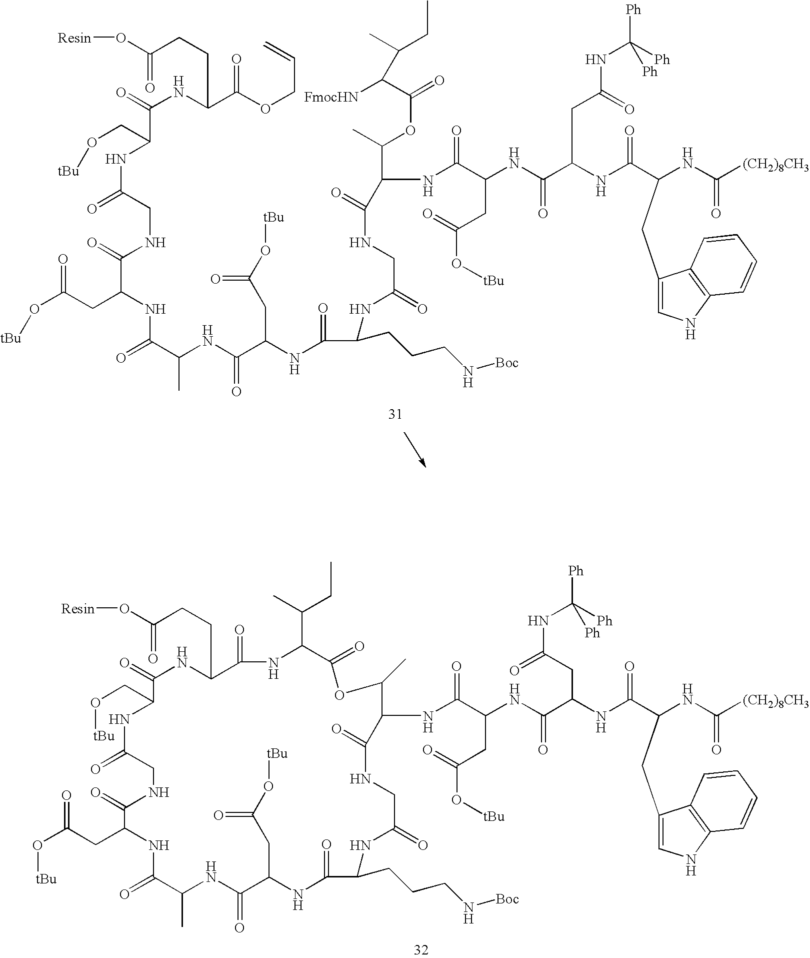

The dried resin 31 was placed under an argon atmosphere, and treated with a solution of tetrakis-(triphenylphosphine)palladium(0) (48 mg in dichloromethane (7.63 mL)), acetic acid (0.38 mL), and N-methylmorpholine (0.19 mL). The mixture was shaken for 4 hours at ambient temperature, filtered through a glass sinter funnel, and the solid was washed two times with N-methylmorpholine, two times with methanol, and again two times with N-methylmorpholine. The solid resin was suspended in 20% piperidine in N-methylmorpholine (7 mL) for 105 minutes, filtered through a glass sinter funnel and the solid was washed well with N-methylmorpholine. 1-Hydroxy-benzotriazole (0.3 mL of a 0.5 molar solution in N-methylmorpholine) and 1,3-diisopropylcarbodiimide (0.3 mL of a 0.5 molar solution in N-methylmorpholine) were added to the resin. The reaction mixture was shaken for 17 hours, filtered through a glass sinter funnel, and the precipitate was washed well with N-methylmorpholine to give the resin bound cyclized depsipeptide 32.

Reaction 4: Preparation of Compound C369

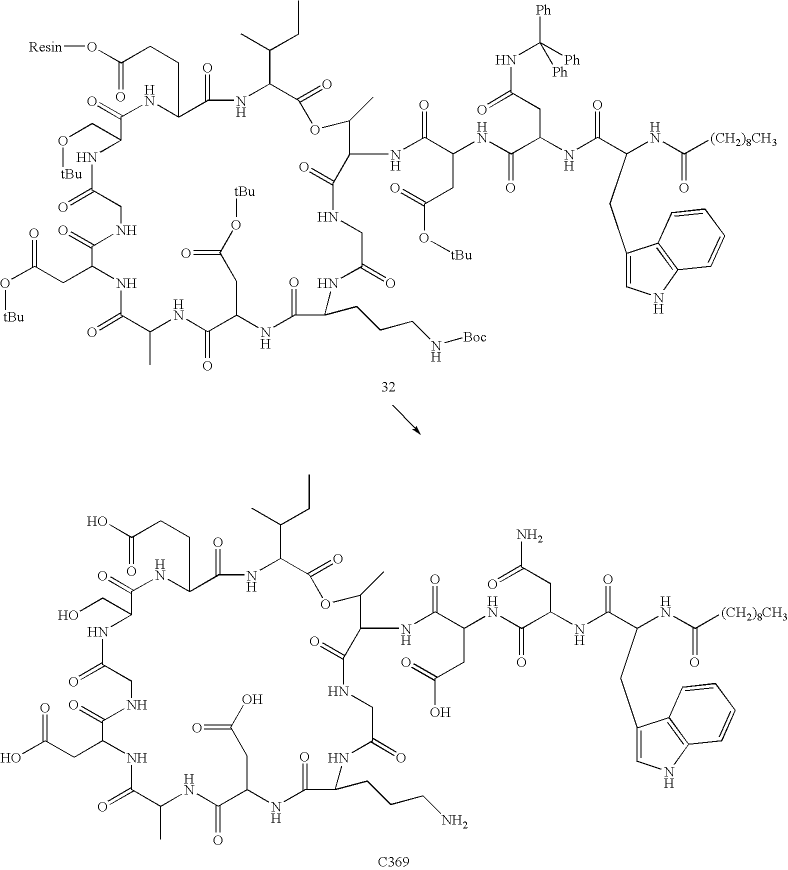

The dried resin 32 was suspended in dichloromethane (4 mL), trifluoroacetic acid (6 mL), ethanedithiol (250 μL), and triisopropylsilane (250 μL), and stirred for 3 hours at ambient temperature. The reaction mixture was filtered through a glass sinter funnel and washed with dichloromethane (2×2 mL) and the combined filtrates were evaporated under reduced pressure. Crude product was then partitioned between diethyl ether (6 mL) and water (3 mL). The aqueous layer was separated and freeze dried to give the crude product 33 (21.5 mgs). The crude product was then purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give the pure product C369 (1.8 mg).

### EXAMPLE 1-6

### Synthesis of Peptide Resin Compound 34

### Resin-Glu(αOAllyl)-DSer(OtBu)-Gly-Asp(OtBu)-DLys(NHBoc)-Asp-(OtBu)NH₂ (34)

### Reaction 1

### Preparation of Resin-Glu(αOAllyl)-DSer(OtBu)-Gly-Asp(OtBu)-DLys(NHBoc)-NHFmoc (35)

Commercially available Nα-(9-Fluorenylmethoxycarbonyl)-Nε-(t-butyloxycarbonyl D-lysine (1.48 g), 1,3-diisopropylcarbodiimide (0.49 mL), 1-hydroxy-benzotriazole (425 mg) and 4-dimethylaminopyridine (37 mg) as a solution in N-methylpyrrolidine (20 mL) was added to resin 17 (vide supra). The reaction mixture was shaken for three hours, filtered through a glass sinter funnel and the coupling was repeated for 15 hours. The reaction mixture was filtered, through a glass sinter funnel then the solid was washed with N-methylpyrrolidine (3×15 mL), methanol (3×15 mL), and again with N-methylpyrrolidine (3×15 mL) to give compound 35.

### Reaction 2

### Preparation of Resin-Glu(αOAllyl)-DSer(OtBu)-Gly-Asp(OtBu)-DLys(HBoc)-NH₂ (36)

Compound 35 was agitated in 20% piperidine in N-methylpyrrolidine (25 mL) for one hour. The reaction mixture was filtered through a glass sinter funnel then the solid was washed with N-methylpyrrolidine (3×15 mL), methanol (3×15 mL), and again with N-methylpyrrolidine (3×15 mL) to give compound 36.

### Reaction 3

### Preparation of Resin-Glu(αOAllyl)-DSer(OtBu)-Gly-Asp(OtBu)-DLys(NHBoc)-Asp(OtBu)-NHFmoc (37)

Commercially available Nα-(9-Fluorenylmethoxycarbonyl)-L-aspartic acid β-tertbutyl ester (2.16 g), 1,3-diisopropylcarbodiimide (822 μL), and 1-hydroxy-benzotriazole (710 mg) as a solution in N-methylpyrrolidine (20 mL) was added to resin 36. The reaction mixture was shaken for four hours. The reaction mixture was filtered through a glass sinter funnel then the solid was washed with N-methylpyrrolidine (3×15 mL), methanol (3×15 mL), and again with N-methylpyrrolidine (3×15 mL) to give compound 37.

### Reaction 4

### Preparation of Resin-Glu(αOAllyl)-DSer(OtBu)-Gly-Asp(OtBu)-DLys(NHBoc)-Asp(OtBu)-NH₂ (34)

Compound 37 was agitated in 20% piperidine in N-methylpyrrolidine (25 mL) for one hour. The reaction mixture was filtered through a glass sinter funnel then the solid was washed with N-methylpyrrolidine (3×15 mL), methanol (3×15 mL), and again with N-methylpyrrolidine (3×15 mL) to give compound 34.

### EXAMPLE 1-7

### Synthesis of Peptide Resin Compound 38

### Resin-Glu(αOAllyl)-DSer(OtBu)-Gly-Asp(OtBu)-DAla-Asp(OtBu)-Ala-NH₂ (38)

### Reaction 1

### Preparation of Resin-Glu(αOAllyl)-DSer(OtBu)-Gly-Asp-(OtBu)-DAla-Asp(OtBu)-Ala-NHFmoc (39)

Commercially available Nα-(9-Fluorenylmethoxycarbonyl)-L-alanine (1.62 g), 1,3-diisopropylcarbodiimide (825 μL), and 1-hydroxy-benzotriazole (715 mg) as a solution in N-methylpyrrolidine (20 mL) was added to resin 21 (vide supra). The reaction mixture was shaken for 17 hours. The reaction mixture was filtered through a glass sinter funnel then the solid was washed with N-methylpyrrolidine (3×15 mL), methanol (3×15 mL), and again with N-methylpyrrolidine (3×15 mL) to give compound 39.

### Reaction 2

### Preparation of Resin-Glu(αOAllyl)-DSer(OtBu)-Gly-Asp(OtBu)-DAla-Asp(OtBu)-Ala-NH₂ (38)

Compound 39 (227 mg) was agitated in 20% piperidine in N-methylpyrrolidine (1 mL) for 0.5 hour. The reaction mixture was filtered through a glass sinter funnel then the solid was washed with N-methylpyrrolidine (3×5 mL), methanol (3×5 mL), and again with N-methylpyrrolidine (3×5 mL) to give 38

### EXAMPLE 1-8

### Synthesis of Peptide Resin Compound 40

### Resin-Glu(αOAllyl)-DAsn(NHTrt)-Gly-Asp(OtBu)-DAla-Asp(OtBu)-NH₂ (40)

### Reaction 1

### Preparation of Resin-Glu(αOAllyl)-DAsn(NHTrt)-NHFmoc (41)

A solution of commercially available Nα-(9-Fluorenylmethoxycarbonyl)-D-asparagine (NHTrt)OH (3.1 g), 2-(1H-Benzotriazol-yl)-1,1,3,3-tetramethyluronium tetrafluoroborate (TBTU, 1.67 g), Hydroxy-benzotriazole (0.56 g) and diisopropylethylamine (DIPEA, 2.7 mL) as a solution in N-methylpyrrolidone (NMP, 40 mL) was added to Resin-Glu-NH₂ (11, vide supra, 4 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×40 mL), methanol (3×40 mL), and again with N-methylpyrrolidine (3×40 mL) to give compound 41.

### Reaction 2

### Preparation of Resin-Glu(αOAllyl)-DAsn(HTrt)-NH (42)

Compound 41 was agitated in 20% piperidine in N-methylpyrrolidine (30 mL) for 30 minutes. The reaction mixture was filtered through a glass sinter funnel and was re-suspended in 20% piperidine in N-methylpyrrolidine (30 mL) and was agitated for 30 minutes. The reaction mixture was filtered through a glass sinter funnel then the solid was washed with N-methylpyrrolidine (3×30 mL), methanol (3×30 mL), and again with N-methylpyrrolidine (3×30 mL) to give compound 42.

### Reaction 3

### Preparation of Resin-Glu(αOAllyl)-DAsn(NHTrt)-Gly-NHFmoc (43)

Commercially available Nα-(9-Fluorenylmethoxycarbonyl)-glycine (1.55 g), 2-(1H-Benzotriazol-yl)-1,1,3,3-tetramethyluronium tetrafluoroborate (TBTU, 1.67 g), Hydroxy-benzotriazole (HOBt, 0.56 g) and diisopropylethylamine (DIPEA, 2.7 mL) as a solution in N-methylpyrrolidone (NMP, 40 mL) was added to compound 42 (4 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×40 mL), methanol (3×40 mL), and again with N-methylpyrrolidine (3×40 mL) to give compound 43.

### Reaction 4

### Preparation of Resin-Glu(αOAllyl)-DAsn(NHTrt)-Gly-NH₂ (44)

Compound 43 was agitated in 20% piperidine in N-methylpyrrolidine (30 mL) for 30 minutes. The reaction mixture was filtered through a glass sinter funnel and was re-suspended in 20% piperidine in N-methylpyrrolidine (30 mL) and agitated for 30 minutes. The reaction mixture was filtered through a glass sinter funnel then the solid was washed with N-methylpyrrolidine (3×30 mL), methanol (3×30 mL), and again with N-methylpyrrolidine (3×30 mL) to give compound 44.

### Reaction 5

### Preparation of Resin-Glu(αOAllyl)-DAsn(NHTrt)-Gly-Asp(OtBu)-NHFmoc (45)

Commercially available Nα-(9-Fluorenylmethoxycarbonyl)-L-aspartic acid α-tertbutyl ester (2.14 g), 2-(1H-Benzotriazol-yl)-1,1,3,3-tetramethyluronium tetrafluoroborate (TBTU, 1.67 g), HOBt (0.56 g) and diisopropylethylamine (DIPEA, 2.7 mL) as a solution in N-methylpyrrolidone (NMP, 40 mL) was added to compound 44 (4 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×40 mL), methanol (3×40 mL), and again with N-methylpyrrolidine (3×40 mL) to give compound 45

### Reaction 6

### Preparation of Resin-Glu(αOAllyl)-DAsn(NHTrt)-Gly-Asp(OtBu)-NH₂ (46)

Compound 45 was agitated in 20% piperidine in N-methylpyrrolidine (30 mL) for 30 minutes. The reaction mixture was filtered through a glass sinter funnel and was re-suspended in 20% piperidine in N-methylpyrrolidine (30 mL) and agitated for 30 minutes. The reaction mixture was filtered through a glass sinter funnel then the solid was washed with N-methylpyrrolidine (3×30 mL), methanol (3×30 mL), and again with N-methylpyrrolidine (3×30 mL) to give compound 46.

### Reaction 7

### Preparation of Resin-Glu(αOAllyl)-DAsn(NHTrt)-Gly-Asp(OtBu)-DAla-NHFmoc (47)

Commercially available Nα-(9-Fluorenylmethoxycarbonyl)-D-alanine (0.81 g), 2-(1H-Benzotriazol-yl)-1,1,3,3-tetramethyluronium tetrafluoroborate (TBTU, 0.84 g), HOBt (0.28 g) and diisopropylethylamine (DIPEA, 1.4 mL) as a solution in N-methylpyrrolidone (NMP, 20 mL) was added to compound 46 (2 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×20 mL), methanol (3×20 mL), and again with N-methylpyrrolidine (3×20 mL) to give compound 47.

### Reaction 8

### Preparation of Glu(αOAllyl)-DAsn(NHTrt)-Gly-Asp(OtBu)-DAla-NH (48)

Compound 47 was agitated in 20% piperidine in N-methylpyrrolidine (15 mL) for 30 minutes. The reaction mixture was filtered through a glass sinter funnel and was re-suspended in 20% piperidine in N-methylpyrrolidine (15 mL) and agitated for 30 minutes. The reaction mixture was filtered through a glass sinter funnel then the solid was washed with N-methylpyrrolidine (3×10 mL), methanol (3×10 mL), and again with N-methylpyrrolidine (3×10 mL) to give compound 48.

### Reaction 9

### Preparation of Resin-Glu(αOAllyl)-DAsn(HTrt)-Gly-Asp(OtBu)-DAla-Asp(OtBu)-NHFmoc (49)

Commercially available Nα-(9-Fluorenylmethoxycarbonyl)-L-aspartic acid β-tertbutyl ester (1.07 g), 2-(1H-Benzotriazol-yl)-1,1,3,3-tetramethyluronium tetrafluoroborate (TBTU, 0.84 g), HOBt (0.28 g) and diisopropylethylamine (DIPEA, 1.4 mL) as a solution in N-methylpyrrolidone (NMP, 20 mL) was added to compound 48 (2 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×20 mL), methanol (3×20 mL), and again with N-methylpyrrolidine (3×20 mL) to give compound 49

### Reaction 10

### Preparation of Glu((αOAllyl)-DAsn(NHTrt)-Gly-Asp(OtBu)-DAla-Asp(OtBu)-NH₉ (40)

Compound 49 was agitated in 20% piperidine in N-methylpyrrolidine (15 mL) for 30 minutes. The reaction mixture was filtered through a glass sinter funnel and was re-suspended in 20% piperidine in N-methylpyrrolidine (15 mL) and agitated for 30 minutes. The reaction mixture was filtered through a glass sinter funnel then the solid was washed with N-methylpyrrolidine (3×10 mL), methanol (3×10 mL), and again with N-methylpyrrolidine (3×10 mL) to give compound 40

### EXAMPLE 1-9

### Synthesis of Peptide Resin Compound 50

### Resin-Glu(αOAllyl)-DAsn(NHTrt)-Gly-Asp(OtBu)-DAla-Asp(OtBu)-Orn(NHBoc)-NH₂ 50

### Reaction 1

### Preparation of Resin-Glu(αOAllyl)-DAsn(NHTrt)-Gly-Asp(OtBu)-DAla-Asp(OtBu)-Orn(NHBoc)-NHFmoc (51)

Commercially available Nα-(9-Fluorenylmethoxycarbonyl)-L-ornithine (Boc)-OH (1.17 g), 2-(1H-Benzotriazol-yl)-1,1,3,3-tetramethyluronium tetrafluoroborate (TBTU, 0.83 g), HOBt (0.31 g) and diisopropylethylamine (DIPEA, 1.4 mL) as a solution in N-methylpyrrolidone (NMP, 20 mL) was added to compound 40 (2.8 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×20 mL), methanol (3×20 mL), and again with N-methylpyrrolidine (3×20 mL) to give compound 51.

### Reaction 2 Preparation of Resin-Glu(αOAllyl)-DAsn(NHTrt)-Gly-Asp(OtBu)-DAla-Asp(OtBu)-Orn(NHBoc)-NH₂ (50)

Compound 51 was agitated in 20% piperidine in N-methylpyrrolidine (15 mL) for 30 minutes. The reaction mixture was filtered through a glass sinter funnel, re-suspended in 20% piperidine in N-methylpyrrolidine (15 mL) and agitated for 30 minutes. The reaction mixture was filtered through a glass sinter funnel then the solid was washed with N-methylpyrrolidine (3×10 mL), methanol (3×10 mL), and again with N-methylpyrrolidine (3×10 mL) to give compound 50.

### EXAMPLE 1-10

### Synthesis of Peptide Resin Compound 52

### Resin-Glu(αOAllyl)-DAsn(NHTrt)-Gly-Asp(OtBu)-DAla-Asp(OtBu)-Ala-NH₂ (52)

### Reaction 1

### Preparation of Resin-Glu(αOAllyl)-DAsn(NHTrt)-Gly-Asp(OtBu)-DAla-Asp(OtBu)-Ala-NHFmoc (53)

Commercially available Nα-(9-Fluorenylmethoxycarbonyl)-L-alanine (63 mg), 2-(1H-Benzotriazol-yl)-1,1,3,3-tetramethyluronium tetrafluoroborate (TBTU, 64 mg), HOBt (27 mg) and diisopropylethylamine (DIPEA, 70 μL) as a solution in N-methylpyrrolidone (NMP, 1 mL) was added to compound 40 (340 mg). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×2 mL), methanol (3×2 mL), and again with N-methylpyrrolidine (3×2 mL) to give compound 53.

Reaction 2: Preparation of Resin-Glu(αOAllyl)-DAsn(NHTrt)-Gly-Asp(OtBu)-DAla-Asp(OtBu)-Ala-NH₂ (52)

Compound 53 was agitated in 20% piperidine in N-methylpyrrolidine (1.5 mL) for 30 minutes. The reaction mixture was filtered through a glass sinter funnel, re-suspended in 20% piperidine in N-methylpyrrolidine (1.5 mL) and agitated for 30 minutes. The reaction mixture was filtered through a glass sinter funnel then the solid was washed with N-methylpyrrolidine (3×1 mL), methanol (3×1 mL), and again with N-methylpyrrolidine (3×1 mL) to give compound 52.

### EXAMPLE 1-11

### Synthesis of Peptide Resin Compound 54

### Resin-Glu(αOAllyl)-DSer(OtBu)-Gly-Asp(OtBu)-DLys(NHBoc)-Asp(OtBu)-Orn(NHBoc)-NH₂ (54)

### Reaction 1

### Preparation of Resin-Glu(αOAllyl)-DSer(OtBu)-Gly-Asp(OtBu)-DLs(NHBoc)-Asp(OtBu)-Orn(NHBoc)-NHFmoc(55)

Commercially available Nα-(9-Fluorenylmethoxycarbonyl)-L-ornithine (Boc)-OH (0.44 g), 2-(1H-Benzotriazol-yl)-1,1,3,3-tetramethyluronium tetrafluoroborate (TBTU, 0.31 g), HOBt (0.13 g) and diisopropylethylamine (DIPEA, 0.3 mL) as a solution in N-methylpyrrolidone (NMP, 20 mL) was added to compound 34 (vide supra, 0.8 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×20 mL), methanol (3×20 mL), and again with N-methylpyrrolidine (3×20 mL) to give compound 55.

### Reaction 2

### Preparation of Resin-Glu(αOAllyl)-DSer(OtBu)-Gly-Asp(OtBu)-DLys(THBoc)-Asp(OtBu)-Orn(NHBoc)-NH₂ (54)

Compound 55 was agitated in 20% piperidine in N-methylpyrrolidine (8 mL) for 30 minutes. The reaction mixture was filtered through a glass sinter funnel, re-suspended in 20% piperidine in N-methylpyrrolidine (8 mL) and agitated for 30 minutes. The reaction mixture was filtered through a glass sinter funnel then the solid was washed with N-methylpyrrolidine (3×8 mL), methanol (3×8 mL), and again with N-methylpyrrolidine (3×8 mL) to give compound 54.

### EXAMPLE 1-12

### Synthesis of Peptide Resin Compound 56

### Resin-Glu(αOAllyl)-DAsn(NHTrt)-Gly-Asp(OtBu)-DLys(NHBoc)-Asp(OtBu)-NH₂ (56)

### Reaction 1

### Preparation of Resin-Glu(αOAllyl)-DAsn(NHTrt)-Gly-Asp(OtBu)-DLys(NHBoc)-NHFmoc (57)

Commercially available Nα-(9-Fluorenylmethoxycarbonyl)-D-Nα-(9-Fluorenylmethoxycarbonyl)-Nε-(t-butyloxycarbonyl L-lysine (1.28 g), 2-(1H-Benzotriazol-yl)-1,1,3,3-tetramethyluronium tetrafluoroborate (TBTU, 0.84 g), HOBt (0.28 g) and diisopropylethylamine (DIPEA, 1.4 mL) as a solution in N-methylpyrrolidone (NMP, 20 mL) was added to compound 46 (2 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×20 mL), methanol (3×20 mL), and again with N-methylpyrrolidine (3×20 mL) to give compound 57.

### Reaction 2

### Preparation of Resin-Glu(αOAllyl)-DAsn(NHTrt)-Gly-Asp(OtBu)-DLys(NHBoc)-NH₂

Compound 57 was agitated in 20% piperidine in N-methylpyrrolidine (15 mL) for 30 minutes. The reaction mixture was filtered through a glass sinter funnel, re-suspended in 20% piperidine in N-methylpyrrolidine (15 mL) and agitated for 30 minutes. The reaction mixture was filtered through a glass sinter funnel then the solid was washed with N-methylpyrrolidine (3×10 mL), methanol (3×10 mL), and again with N-methylpyrrolidine (3×10 mL) to give compound 58.

### Reaction 3

### Preparation of Resin-Glu(αOAllyl)-DAsn(NHTrt)-Gly-AsM(OtBu)-DLys(NHBoc)-Asp(OtBu)-NHFmoc (59)

Commercially available Nα-(9-Fluorenylmethoxycarbonyl)-L-aspartic acid β-tertbutyl ester (1.07 g), 2-(1H-Benzotriazol-yl)-1,1,3,3-tetramethyluronium tetrafluoroborate (TBTU, 0.84 g), HOBt (0.28 g) and diisopropylethylamine (DIPEA, 1.4 mL) as a solution in N-methylpyrrolidone (NMP, 20 mL) was added to compound 58 (2 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×20 mL), methanol (3×20 mL), and again with N-methylpyrrolidine (3×20 mL) to give compound 591

### Reaction 4

### Preparation of Resin-Glu(αOAllyl)-DAsn(NHTrt)-Gly-Asp(OtBu)-DLys(NHBoc)-Asp(OtBu)-NH₂ (56)

Compound 59 was agitated in 20% piperidine in N-methylpyrrolidine (15 mL) for 30 minutes. The reaction mixture was filtered through a glass sinter funnel, re-suspended in 20% piperidine in N-methylpyrrolidine (15 mL) and agitated for 30 minutes. The reaction mixture was filtered through a glass sinter funnel then the solid was washed with N-methylpyrrolidine (3×10 mL), methanol (3×10 mL), and again with N-methylpyrrolidine (3×10 mL) to give compound 56.

### EXAMPLE 1-13

### Synthesis of Peptide Resin Compound 60

### Resin-Glu(αOAllyl)-DAsn(NHTrt)-Gly-Asp(OtBu)-DLys(NHBoc)-Asp(OtBu)-Orn(NHBoc)-NH (60)

### Reaction 1

### Preparation of Resin-Glu(αOAllyl)-DAsn(NHTrt)-Gly-Asp(OtBu)-DLys(HBoc)-Asp(OtBu)-Orn(NHBoc)-NHFmoc (61)

Commercially available Nα-(9-Fluorenylmethoxycarbonyl)-L-ornithine (Boc)-OH (0.54 g), 2-(1H-Benzotriazol-yl)-1,1,3,3-tetramethyluronium tetrafluoroborate (TBTU, 0.38 g), HOBt (0.12 g) and diisopropylethylamine (DIPEA, 0.63 mL) as a solution in N-methylpyrrolidone (NMP, 12 mL) was added to compound 56 (1.2 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×20 mL), methanol (3×20 mL), and again with N-methylpyrrolidine (3×20 mL) to give compound 61.

### Reaction 2

### Preparation of Resin-Glu(αOAllyl)-DAsn(NHTrt)-Gly-Asp(OtBu)-DLys(NHBoc)-Asp(OtBu)-Orn(NHBoc)-NH₂ (60)

Compound 61w as agitated in 20% piperidine in N-methylpyrrolidine (12 mL) for 30 minutes. The reaction mixture was filtered through a glass sinter funnel, re-suspended in 20% piperidine in N-methylpyrrolidine (12 mL) and agitated for 30 minutes. The reaction mixture was filtered through a glass sinter funnel then the solid was washed with N-methylpyrrolidine (3×10 mL), methanol (3×10 mL), and again with N-methylpyrrolidine (3×10 mL) to give compound 60.

### EXAMPLE 1-14

### Synthesis of Peptide Resin Compound 62

### Resin-Glu(αOAllyl)-DAsn(NHTrt)-Gly-Asp(OtBu)-DLys(NHBoc)-Asp(OtBu)-Ala-NH₂ (62)

### Reaction 1

### Preparation of Resin-Glu(αOAllyl)-DAsn(NHTrt)-Gly-Asp(OtBu)-DLys(HBoc)-Asp(OtBu)-Ala-NHFmoc(63)

Commercially available Nα-(9-Fluorenylmethoxycarbonyl)-L-alanine (0.78 g), 2-(1H-Benzotriazol-yl)-1,1,3,3-tetramethyluronium tetrafluoroborate (TBTU, 0.80 g), HOBt (0.27 g) and diisopropylethylamine (DIPEA, 0.81 mL) as a solution in N-methylpyrrolidone (NMP, 20 mL) was added to compound 56 (2 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×20 mL), methanol (3×20 mL), and again with N-methylpyrrolidine (3×20 mL) to give compound.63.

### Reaction 2

### Preparation of Resin-Glu(αOAllyl)-DAsn(NHTrt)-Gly-Asp(OtBu)-DLys(NHBoc)-Asp(OtBu)-Ala-NH₂ (62)

Compound 63 was agitated in 20% piperidine in N-methylpyrrolidine (20 mL) for 30 minutes. The reaction mixture was filtered through a glass sinter funnel, re-suspended in 20% piperidine in N-methylpyrrolidine (20 mL) and agitated for 30 minutes. The reaction mixture was filtered through a glass sinter funnel then the solid was washed with N-methylpyrrolidine (3×20 mL), methanol (3×20 mL), and again with N-methylpyrrolidine (3×20 mL) to give compound 62.

### EXAMPLE 1-15

### Synthesis of Peptide Resin Compound 64

### Resin-Ala-Sar-Thr-Asp(OtBu)-DAsn(NHTrt)-Trp-NH₂ (64)

### Reaction 1

### Preparation of Resin-Ala-Sar-NMeFmoc (65)

A solution of commercially available Nα-(9-Fluorenylmethoxycarbonyl)-sarcosine (1.56 g), 2-(1H-Benzotriazol-yl)-1,1,3,3-tetramethyluronium tetrafluoroborate (TBTU, 1.61 g), and diisopropylethylamine (DIPEA, 871 μl) as a solution in N-methylpyrrolidone (NMP, 25 mL) was added to commercially available alanine 2-chlorotrityl resin (66, 2.5 g). The mixture was shaken for 30 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×15 mL), methanol (3×15 mL), and again with N-methylpyrrolidine (3×15 mL) to give compound 65.

### Reaction 2

### Preparation of Resin-Ala-Sar-NMeH (67)

Compound 65 was agitated in 20% piperidine in N-methylpyrrolidine (20 mL) for 1 hour. The reaction mixture was filtered through a glass sinter funnel then the solid washed with N-methylpyrrolidine (3×15 mL), methanol (3×15 mL), and again with N-methylpyrrolidine (3×15 mL) to give compound 67.

### Reaction 3

### Preparation of Resin-Ala-Sar-Thr-NHFmoc (68)

Commercially available Nα-(9-Fluorenylmethoxycarbonyl)-L-threonine (853 mg), bromo-tris-pyrrolidinophosphonium hexafluorophosphate (PyBrOP, 1.165 g), and DIPEA (1.31 mL) as a solution in dichloromethane (25 mL) was added to compound 67 (334 mg). The mixture was shaken for one hour. The reaction mixture was filtered through a glass sinter funnel then the solid was washed with N-methylpyrrolidine (3×15 mL), methanol (3×15 mL), and again with N-methylpyrrolidine (3×15 mL) to give compound 68.

### Reaction 4

### Preparation of Resin-Ala-Sar-Thr-NH₂ (69)

Compound 38 (vide supra) was agitated in 20% piperidine in N-methylpyrrolidine (25 mL) for 1 hour. The reaction mixture was filtered through a glass sinter funnel then the solid was washed with N-methylpyrrolidine (3×15 mL), methanol (3×15 mL), and again with N-methylpyrrolidine (3×15 mL) to give compound 69.

### Reaction 5

### Preparation of Resin-Ala-Sar-Thr-Asp(OtBu)-NHFmoc (70)

Commercially available Nα-(9-Fluorenylmethoxycarbonyl)-L-aspartic acid β-tert-butyl ester (2.06 g), TBTU (1.61 g), and DIPEA (871 μL) as a solution in NMP (25 mL) were added to compound 69. The mixture was shaken for three hours. The reaction mixture was filtered through a glass sinter funnel then the solid was washed with N-methylpyrrolidine (3×15 mL), methanol (3×15 mL), and again with N-methylpyrrolidine (3×15 mL) to give compound 70.

### Reaction 6

### Preparation of Resin-Ala-Sar-Thr-Asp(OtBu)-NH₂ (71)

Compound 70 was agitated in 20% piperidine in N-methylpyrrolidine (25 mL) for one hour. The reaction mixture was filtered through a glass sinter funnel then washed with N-methylpyrrolidine (3×15 mL), methanol (3×15 mL), and again with N-methylpyrrolidine (3×15 mL) to give compound 71.

### Reaction 7

### Preparation of Resin-Ala-Sar-Thr-Asp(OtBu)-DAsn(NHTrt)-NHFmoc (72)

Commercially available Nα-(9-Fluorenylmethoxycarbonyl)-D-asparagine δ-N-trityl (1.49 g), TBTU (1.61 g), and DIPEA (871 μL) as a solution in NMP (25 mL) was added to compound 71. The reaction mixture was shaken for seventeen hours. The reaction mixture was filtered through a glass sinter funnel then the solid was washed with N-methylpyrrolidine (3×15 mL), methanol (3×15 mL), and again with N-methylpyrrolidine (3×15 mL) to give compound 72.

### Reaction 8

### Preparation of Resin-Ala-Sar-Thr-Asp(OtBu)-DAsn(HTrt)-NH₂ (73)

Compound 72 was agitated in 20% piperidine in N-methylpyrrolidine (25 mL) for 2 hours. The reaction mixture was filtered through a glass sinter funnel then the solid was washed with N-methylpyrrolidine (3×15 mL), methanol (3×15 mL), and again with N-methylpyrrolidine (3×15 mL) to give compound 73.

### Reaction 9

### Preparation of Resin-Ala-Sar-Thr-Asp(OtBu)-DAsn(NHTrt)-Trp-NHFmoc (74)

Commercially available Nα-(9-Fluorenylmethoxycarbonyl)-L-tryptophan (1.07 g), TBTU (802 mg), and DIPEA (435 μL) as a solution in NMP (10 mL) was added to resin 73. The reaction mixture was shaken for forty three hours. The reaction mixture was filtered through a glass sinter funnel then the solid was washed with N-methylpyrrolidine (3×15 mL), methanol (3×15 mL), and again with N-methylpyrrolidine (3×15 mL) to give compound 74.

### Reaction 10

### Preparation of Resin-Ala-Sar-Thr-Asp(OtBu)-DAsn(NHTrt)-Trp-NH₂ (64)

Compound 74 was agitated in 20% piperidine in N-methylpyrrolidine (25 mL) for one hour. The reaction mixture was filtered through a glass sinter funnel then the solid was washed with N-methylpyrrolidine (3×15 mL), methanol (3×15 mL), and again with N-methylpyrrolidine (3×15 mL) to give resin peptide compound 64.

### EXAMPLE 1-16

### Synthesis of Peptide Resin Compound (75)

### Resin-Ala-Sar-Thr-Asp(OtBu)-DAsn(NlTrt)-Trp-Undecanoic amide. (75)

Commercially available undecanoic acid (930 mg), 1,3-diisopropylcarbodiimide (0.78 mL), and 1-hydroxy-benzotriazole (676 mg) as a solution in N-methylpyrrolidine (20 mL) was added to compound 64. The mixture was shaken for 23 hours, filtered through a glass sinter funnel, and the reaction was judged to be incomplete using the Kaiser Test (vide supra). The resin was then filtered through a glass sinter funnel and washed with N-methylpyrrolidine (3×15 mL), methanol (3×15 mL), and again with N-methylpyrrolidine (3×15 mL). The reaction was found to be complete using the Kaiser Test, yielding the resin bound compound 75.

### EXAMPLE 1-17

### Synthesis of Peptide Resin Compound (76)

### Resin-Gly-Thr-Asp(OtBu)-DAsn(NHTrt)-Trp-8-Methyldecanoic amide (76)

Commercially available 8-methyldecanoic acid (1.55 g), 2-(1H-Benzotriazol-yl)-1,1,3,3-tetramethyluronium tetrafluoroborate (TBTU, 2.67 g), diisopropylethylamine (DIPEA, 2.9 mL), and 1-hydroxy-benzotriazole (1.12 g) as a solution in N-methylpyrrolidine (80 mL) was added to compound 1 (7.6 g). The mixture was shaken for 18 hours, filtered through a glass sinter funnel, and the reaction was judged to be complete using the Kaiser Test (vide supra), yielding the resin bound compound 76.

### EXAMPLE 1-18

### Synthesis of Peptide Resin Compound (77)

### Resin-Gly-Thr-Asp(OtBu)-DAsn(NHTrt)-Trp-tridecanoic amide (77)

Commercially available tridecanoic acid (2.39 g), 2-(1H-Benzotriazol-yl)-1,1,3,3-tetramethyluronium tetrafluoroborate (TBTU, 3.47 g), diisopropylethylamine (DIPEA, 3.75 mL), and 1-hydroxy-benzotriazole (1.46 g) as a solution in N-methylpyrrolidine (80 mL) was added to compound 1 (10 g). The mixture was shaken for 17 hours, filtered through a glass sinter funnel, and the reaction was judged to be complete using the Kaiser Test (vide supra), yielding the resin bound compound 77.

### EXAMPLE 1-19

### Synthesis of Peptide Resin Compound (78)

### Resin-Gly-Thr-Asp(OtBu)-DGlu(OtBu)-Trp-8-Methyldecanoic amide (78)

### Reaction 1

### Preparation of Resin-Gly-Thr-Asp(OtBu)-DGlu(OtBu)-NHFmoc (79)

Commercially available Nα-(9-Fluorenylmethoxycarbonyl)-D-glutamic acid 7-t-butyl ester (1.14 g), TBTU (0.87 g), HOBt (0.37 g) and DIPEA (940 μL) as a solution in NMP (20 mL) was added to compound 5. The reaction mixture was shaken for one hour. The reaction mixture was filtered through a glass sinter funnel then the solid was washed with N-methylpyrrolidine (3×15 mL), methanol (3×15 mL), and again with N-methylpyrrolidine (3×15 mL). The reaction was judged to be complete using the Kaiser Test (vide supra), yielding the resin bound compound 79.

### Reaction 2

### Preparation of Resin-Gly-Thr-Asp(OtBu)-DGlu(OtBu)-NH₂ (80)

Compound 79 was agitated in 20% piperidine in N-methylpyrrolidine (20 mL) for 15 minutes. The resin was filtered through a glass sinter funnel and re-suspended in 20% piperidine in N-methylpyrrolidine (20 mL) and agitated for 15 minutes. The reaction mixture was filtered through a glass sinter funnel then the solid was washed with N-methylpyrrolidine (3×15 mL), methanol (3×15 mL), and again with N-methylpyrrolidine (3×15 mL) to give resin bound compound 80.

### Reaction 3

### Preparation of Resin-Gly-Thr-Asp(OtBu)-DGlu(OtBu)-Trp-NHFmoc

Commercially available Nα-(9-Fluorenylmethoxycarbonyl)-L-tryptophan (1.15 g), TBTU (0.87 g), HOBt (0.37 g) and DIPEA (940 μL) as a solution in NMP (20 mL) was added to the compound 80. The reaction mixture was shaken for one hour. The reaction mixture was filtered through a glass sinter funnel then the solid was washed with N-methylpyrrolidine (3×15 mL), methanol (3×15 mL), and again with N-methylpyrrolidine (3×15 mL). The reaction was judged to be complete using the Kaiser Test (vide supra), yielding the resin bound 81.

### Reaction 4

### Preparation of Resin-Gly-Thr-Asp(OtBu)-DGlu(OtBu)-Trp-NH₂ (82)

Resin bound compound 81 was agitated in 20% piperidine in N-methylpyrrolidine (20 mL) for 15 minutes. The resin was filtered through a glass sinter funnel and re-suspended in 20% piperidine in N-methylpyrrolidine (20 mL) and agitated for 15 minutes. The reaction mixture was filtered through a glass sinter funnel then the solid was washed with N-methylpyrrolidine (3×15 mL), methanol (3×15 mL), and again with N-methylpyrrolidine (3×15 mL) to give resin bound compound 82.

### Reaction 5

### Preparation of Resin-Gly-Thr-Asp(OtBu)-DGlu(OtBu)-TrM-8-Methyldecanoic amide (78)

Commercially available 8-methyldecanoic acid (0.71 g), 2-(1H-Benzotriazol-yl)-1,1,3,3-tetramethyluronium tetrafluoroborate (TBTU, 1.21 g), diisopropylethylamine (DIPEA, 2.0 mL), and 1-hydroxy-benzotriazole (0.508 g) as a solution in N-methylpyrrolidine (80 mL) was added to compound 82 (4.0 g). The mixture was shaken for 18 hours, filtered through a glass sinter funnel), and the reaction was judged to be complete using the Kaiser Test (vide supra). The reaction mixture was filtered through a glass sinter funnel then the solid was washed with N-methylpyrrolidine (3×6 mL), methanol (3×6 mL), and again with N-methylpyrrolidine (3×6 mL) to give resin bound compound 78.

### EXAMPLE 1-20

### Synthesis of Peptide Resin Compound (83)

### Resin-Ala-Sar-Thr-Asp(OtBu)-DAsn(NHTrt)-Trp-8-Methyldecanoic amide (83)

Commercially available 8-methyldecanoic acid (0.71 g), 2-(1H-Benzotriazol-yl)-1,1,3,3-tetramethyluronium tetrafluoroborate (TBTU, 0.60 g), diisopropylethylamine (DIPEA, 0.64 mL), and 1-hydroxy-benzotriazole (0.25 g) as a solution in N-methylpyrrolidine (20 mL) was added to compound 34 (1.8 g). The mixture was shaken for 18 hours, filtered through a glass sinter funnel, and the reaction was judged to be complete using the Kaiser Test (vide supra). The reaction mixture was filtered through a glass sinter funnel then the solid was washed with N-methylpyrrolidine (3×6 mL), methanol (3×6 mL), and again with N-methylpyrrolidine (3×6 mL) to give resin bound compound 83.

### EXAMPLE 1-21

### Synthesis of Peptide Resin Compound (84)

### Resin-Ala-Sar-Thr-Asp(OtBu)-DGlu(OtBu)-Trp-8-Methyldecanoic amide (84)

### Reaction 1

### Preparation of Resin-Ala-Sar-Thr-Asp(OtBu)-DGlu(OtBu)-NHFmoc (85)

Commercially available Nα-(9-Fluorenylmethoxycarbonyl)-D-glutamic acid γ-t-butyl ester (0.98 g), TBTU (0.74 g), HOBt (0.31 g) and DIPEA (810 μL) as a solution in NMP (20 mL) was added to compound 71 (1.8 g). The reaction mixture was shaken for seventeen hours. The reaction mixture was filtered through a glass sinter funnel then the solid was washed with N-methylpyrrolidine (3×15 mL), methanol (3×15 mL), and again with N-methylpyrrolidine (3×15 mL) to give compound 85.

### Reaction 2

### Preparation of Resin-Ala-Sar-Thr-Asp(OtBu)-DGlu(OtBu) NH (86)

Compound 85 was agitated in 20% piperidine in N-methylpyrrolidine (20 mL) for 30 minutes. The reaction mixture was filtered through a glass sinter funnel, re-suspended in 20% piperidine in N-methylpyrrolidine (20 mL) and agitated for 30 minutes. The reaction mixture was filtered through a glass sinter funnel then the solid was washed with N-methylpyrrolidine (3×20 mL), methanol (3×20 mL), and again with N-methylpyrrolidine (3×20 mL) to give compound 86.

### Reaction 3

### Preparation of Resin-Ala-Sar-Thr-Asp(OtBu)-DGlu(OtBu)-Trp-NHFmoc (87)

Commercially available Ncc-(9-Fluorenylmethoxycarbonyl)-L-tryptophan (0.98 g), TBTU (0.74 g), HOBt (0.31 g) and DIPEA (810 μL) as a solution in NMP (25 mL) was added to compound 86 (2.2 g). The reaction mixture was shaken for seventeen hours. The reaction mixture was filtered through a glass sinter funnel then the solid was washed with N-methylpyrrolidine (3×25 mL), methanol (3×25 mL), and again with N-methylpyrrolidine (3×25 mL) to give compound 87.

### Reaction 4

### Preparation of Resin-Ala-Sar-Thr-Asp(OtBu)-DGlu(OtBu)-Trp-NH₂ (88)

Compound 87 was agitated in 20% piperidine in N-methylpyrrolidine (25 mL) for 30 minutes. The reaction mixture was filtered through a glass sinter funnel, re-suspended in 20% piperidine in N-methylpyrrolidine (25 mL) and agitated for 30 minutes. The reaction mixture was filtered through a glass sinter funnel then the solid was washed with N-methylpyrrolidine (3×30 mL), methanol (3×30 mL), and again with N-methylpyrrolidine (3×30 mL) to give compound 88.

### Reaction 5

### Preparation of Resin-Ala-Sar-Thr-Asp(OtBu)-DGlu(OtBu)-Trp-8-Methyldecanoic amide (84)

Commercially available 8-methyldecanoic acid (0.34 g), 2-(1H-Benzotriazol-yl)-1,1,3,3-tetramethyluronium tetrafluoroborate (TBTU, 0.60 g), diisopropylethylamine (DIPEA, 0.64 mL), and 1-hydroxy-benzotriazole (0.25 g) as a solution in N-methylpyrrolidine (20 mL) was added to compound 88 (2.0 g). The mixture was shaken for 18 hours, filtered through a glass sinter funnel, and the reaction was judged to be complete using the Kaiser Test (vide supra). The solid was washed with N-methylpyrrolidine (3×15 mL), methanol (3×16 mL), and again with N-methylpyrrolidine (3×16 mL) to give resin bound compound 84.

### EXAMPLE 1-22

### Synthesis of Peptide Resin Compound 89

### Resin-Ala-Gly-Thr-Asp(OtBu)-DAsn(NHTrt)-Trp-Undecanoic amide (89)

### Reaction 1

### Preparation of Resin-Ala-Gly-NHFmoc (90)

A solution of commercially available Nα-(9-Fluorenylmethoxycarbonyl)-glycine (1.49 g), TBTU (1.61 g), and DIPEA (871 μL) as a solution in NMP (25 mL) were added to the commercially available Alanine-2-cholrotrityl-resin (66, 2.5 g). The mixture was shaken for three hours, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×15 mL), methanol (3×15 mL), and again with N-methylpyrrolidine (3×15 mL) to give compound 90.

### Reaction 2

### Preparation of Resin-Ala-Gly-NH₂ (91)

Compound 90 was agitated in 20% piperidine in N-methylpyrrolidine (20 mL) for 1 hour. The reaction mixture was filtered through a glass sinter funnel then the solid washed with N-methylpyrrolidine (3×15 mL), methanol (3×15 mL), and again with N-methylpyrrolidine (3×15 mL) to give compound 91.

### Reaction 3

### Preparation of Resin-Ala-Gly-Thr-NHFmoc (92)

Commercially available Ncc-(9-Fluorenylmethoxycarbonyl)-L-threonine (853 mg), bromo-tris-pyrrolidinophosphonium hexafluorophosphate (PyBrOP, 1.165 g), and DIPEA (1.31 mL) as a solution in dichloromethane (25 mL) was added to compound 91 (334 mg). The mixture was shaken for one hour. The reaction mixture was filtered through a glass sinter funnel then the solid was washed with N-methylpyrrolidine (3×15 mL), methanol (3×15 mL), and again with N-methylpyrrolidine (3×15 mL) to give compound 92.

### Reaction 4

### Preparation of Resin-Ala-Gly-Thr-NH₂ (93)

Compound 92 was agitated in 20% piperidine in N-methylpyrrolidine (20 mL) for 1.5 hours. The reaction mixture was filtered through a glass sinter funnel then the solid washed with N-methylpyrrolidine (3×15 mL), methanol (3×15 mL), and again with N-methylpyrrolidine (3×15 mL) to give resin bound compound 93.

### Reaction 5

### Preparation of Resin-Ala-Gly-Thr-Asp(OtBu)-NHFmoc (94)

Commercially available Nα-(9-Fluorenylmethoxycarbonyl)-L-aspartic acid β-tert-butyl ester (2.06 g), TBTU (1.61 g), and DIPEA (871 μL) as a solution in NMP (25 mL) was added to compound 93. The mixture was shaken for three hours. The reaction mixture was filtered through a glass sinter funnel then the solid was washed with N-methylpyrrolidine (3×15 mL), methanol (3×15 mL), and again with N-methylpyrrolidine (3×15 mL) to give compound 94.

### Reaction 6

### Preparation of Resin-Ala-Gly-Thr-Asp(OtBu)-NH₂ (95)

Compound 94 was agitated in 20% piperidine in N-methylpyrrolidine (20 mL) for 1 hour. The reaction mixture was filtered through a glass sinter funnel then the solid washed with N-methylpyrrolidine (3×15 mL), methanol (3×15 mL), and again with N-methylpyrrolidine (3×15 mL) to give compound 94.

### Reaction 7

### Preparation of Resin-Ala-Gly-Thr-Asp-(OtBu)-DAsn(NHTrt)-NHFmoc (96)

Commercially available Nα-(9-Fluorenylmethoxycarbonyl)-D-asparagine δ-N-trityl (1.49 g), TBTU (0.80 g), and DIPEA (435 μL) as a solution in DMF (10 mL) were added to compound 95. The mixture was shaken seventeen hours. The reaction mixture was filtered through a glass sinter funnel then the solid was washed with N-methylpyrrolidine (3×15 mL), methanol (3×15 mL), and again with N-methylpyrrolidine (3×15 mL) to give compound 96.

### Reaction 8

### Preparation of Resin-Ala-Gly-Thr-Asp(OtBu)-DAsn(NHTrt)-NH₂ (97)

Compound 96 was agitated in 20% piperidine in N-methylpyrrolidine (20 mL) for 2 hours. The reaction mixture was filtered through a glass sinter funnel then the solid washed with N-methylpyrrolidine (3×15 mL), methanol (3×15 mL), and again with N-methylpyrrolidine (3×15 mL) to give compound 97.

### Reaction 9

### Preparation of Resin-Ala-Gly-Thr-Asp(OtBu)-DAsn(NHTrt)-Trp-NHFmoc (98)

Commercially available Ncc-(9-Fluorenylmethoxycarbonyl)-L-tryptophan (1.07 g), TBTU (0.80 g), and DIPEA (435 μL) as a solution in NMP (25 mL) was added to compound 97. The mixture was shaken for forty three hours. The reaction mixture was filtered through a glass sinter funnel then the solid was washed with N-methylpyrrolidine (3×15 mL), methanol (3×15 mL), and again with N-methylpyrrolidine (3×15 mL) to give compound 98.

### Reaction 10

### Preparation of Resin-Ala-Gly-Thr-Asp(OtBu)-DAsn(NHTrt)-Trp-NH₂ (99)

Compound 98 was agitated in 20% piperidine in N-methylpyrrolidine (20 mL) for 1 hour. The reaction mixture was filtered through a glass sinter funnel then the solid washed with N-methylpyrrolidine (3×15 mL), methanol (3×15 mL), and again with N-methylpyrrolidine (3×15 mL) to give compound 99.

### Reaction 11

### Preparation of Resin-Ala-Gly-Thr-Asp(OtBu)-DAsn(NHTrt)-Tpr-Undecanoic amide (89)

Commercially available undecanoic acid (930 mg), 1,3-diisopropylcarbodiimide (0.78 mL), and 1-hydroxy-benzotriazole (676 mg) as a solution in N-methylpyrrolidine (20 mL) was added to compound 99. The mixture was shaken for 23 hours, filtered through a glass sinter funnel, and the reaction was judged to be incomplete using the Kaiser Test (vide supra). The resin was then filtered through a glass sinter funnel and washed with N-methylpyrrolidine (3×15 mL), methanol (3×15 mL), and again with N-methylpyrrolidine (3×15 mL). The reaction was found to be complete using the Kaiser Test, yielding compound 89.

### EXAMPLE 1-23

### Synthesis of peptide Resin Compound 100

### Resin-Ala-Gly-Thr-Asp(OtBu)-DGlu(OtBu)-Trp-Undecanoic amide (100)

### Reaction 1

### Preparation of Resin-Ala-Gly-Thr-Asp(OtBu)-DGlu(OtBu)-NHFmoc. (101)

Commercially available Nα-(9-Fluorenylmethoxycarbonyl)-D-glutamic acid γ-t-butyl ester (1.06 g), TBTU (0.80 g), and DIPEA (435 μL) as a solution in DMF (10 mL) were added to compound 95. The mixture was shaken seventeen hours. The reaction mixture was filtered through a glass sinter funnel then the solid was washed with N-methylpyrrolidine (3×15 mL), methanol (3×15 mL), and again with N-methylpyrrolidine (3×15 mL) to give compound compound 101.

### Reaction 2

### Preparation of Resin-Ala-Gly-Thr-Asp(OtBu)-DGlu(OtBu)-NH₂ (102)

Compound 101 was agitated in 20% piperidine in N-methylpyrrolidine (20 mL) for 1 hour. The reaction mixture was filtered through a glass sinter funnel then the solid washed with N-methylpyrrolidine (3×15 mL), methanol (3×15 mL), and again with N-methylpyrrolidine (3×15 mL) to give compound 102.

### Reaction 3

### Preparation of Resin-Ala-Gly-Thr-Asp(OtBu)-DGlu(OtBu)-Trp-NHFmoc (103)

Commercially available Nα-(9-Fluorenylmethoxycarbonyl)-L-tryptophan (1.07 g), TBTU (0.80 g), and DIPEA (435 μL) as a solution in NMP (25 mL) was added to compound 102. The mixture was shaken for forty three hours. The reaction mixture was filtered through a glass sinter funnel then the solid was washed with N-methylpyrrolidine (3×15 mL), methanol (3×15 mL), and again with N-methylpyrrolidine (3×15 mL) to give 103.

### Reaction 4

### Preparation of Resin-Ala-Gly-Thr-Asp(OtBu)-DGlu(OtBu)-Trp-NH₂ (104)

Compound 103 was agitated in 20% piperidine in N-methylpyrrolidine (20 mL) for 1 hour. The reaction mixture was filtered through a glass sinter funnel then the solid washed with N-methylpyrrolidine (3×15 mL), methanol (3×15 mL), and again with N-methylpyrrolidine (3×15 mL) to give compound 104.

### Reaction 5

### Preparation of Resin-Ala-Gly-Thr-Asp(OtBu)-DGlu(OtBu)-Trp Undecanoic amide. (100)

Commercially available undecanoic acid (930 mg), 1,3-diisopropylcarbodiimide (0.78 mL), and 1-hydroxy-benzotriazole (676 mg) as a solution in N-methylpyrrolidine (20 mL) was added to compound 104. The mixture was shaken for 23 hours, filtered through a glass sinter funnel, and the reaction was judged to be incomplete using the Kaiser Test (vide supra). The resin was then filtered through a glass sinter funnel and washed with N-methylpyrrolidine (3×15 mL), methanol (3×15 mL), and again with N-methylpyrrolidine (3×15 mL). The reaction was found to be complete using the Kaiser Test, yielding the compound 100.

### EXAMPLE 1-24

### Synthesis of Peptide Resin 105

### Resin-Orn(NHIBoc)-Sar-Thr-Asp(OtBu)-DAsn(NHTrt)-Trp-8-Methyldecanoic amide (105)

### Reaction 1

### Preparation of Resin-Orn(NHBoc)-NHFmoc (106)

Commercially available Nα-(9-Fluorenylmethoxycarbonyl)-N-δ-tertbutoxycarbonyl-L-ornithine (8.73 g) as a solution in dichloromethane (100 mL) and diisopropylethylamine (DIPEA, 13.4 mL), were added to a pre-swollen commercially available 2-chlorotrityl resin 107 (10.0 g). The mixture was shaken for 1 hour, filtered through a glass sinter funnel, and the reaction was judged to be complete using the Kaiser Test (vide supra). The reaction mixture was filtered through a glass sinter funnel then the solid was washed with N-methylpyrrolidine (3×100 mL), methanol (3×100 mL), and again with N-methylpyrrolidine (3×100 mL) to give compound 106.

### Reaction 2

### Preparation of Resin-Orn(NHBoc)-NH₂ (108)

Compound 106 was agitated in 20% piperidine in N-methylpyrrolidine (100 mL) for 30 minutes. The reaction mixture was filtered through a glass sinter funnel, re-suspended in 20% piperidine in N-methylpyrrolidine (100 mL) and agitated for 30 minutes. The reaction mixture was filtered through a glass sinter funnel then the solid was washed with N-methylpyrrolidine (3×130 mL), methanol (3×130 mL), and again with N-methylpyrrolidine (3×130 mL) to give compound 108.

### Reaction 3

### Preparation of Resin-Orn(NHBoc)-Sar-NMeFmoc (109)

A solution of commercially available Nα-(9-Fluorenylmethoxycarbonyl)-sarcosine (2.6 g), 2-(1H-Benzotriazol-yl)-1,1,3,3-tetramethyluronium tetrafluoroborate (TBTU, 2.7 g), HOBt (1.13 g) and diisopropylethylamine (DIPEA, 2.9 mL) as a solution in N-methylpyrrolidone (100 mL) was added to compound 108 (10 g). The mixture was shaken for 60 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×115 mL), methanol (3×115 mL), and again with N-methylpyrrolidine (3×115 mL) to give compound 109.

### Reaction 4

### Preparation of Resin-Orn(NHBoc)-Sar-NMeH (110)

Compound 109 was agitated in 20% piperidine in N-methylpyrrolidine (100 mL) for 30 minutes. The reaction mixture was filtered through a glass sinter funnel, re-suspended in 20% piperidine in N-methylpyrrolidine (100 mL) and agitated for 30 minutes. The reaction mixture was filtered through a glass sinter funnel then the solid was washed with N-methylpyrrolidine (3×130 mL), methanol (3×130 mL), and again with N-methylpyrrolidine (3×130 mL) to give compound 110.

### Reaction 5

### Preparation of Resin-Orn(NHBoc)-Sar-Thr-NHFmoc (111)

Commercially available Nα-(9-Fluorenylmethoxycarbonyl)-L-threonine (2.9 g), 2-(1H-Benzotriazol-yl)-1,1,3,3-tetramethyluronium tetrafluoroborate (TBTU, 2.7 g), HOBt (1.13 g) and diisopropylethylamine (DIPEA, 2.9 mL) as a solution in N-methylpyrrolidone (100 mL) was added to compound 110 (11 g). The mixture was shaken for 60 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×115 mL), methanol (3×115 mL), and again with N-methylpyrrolidine (3×115 mL) to give compound 111.

### Reaction 6

### Preparation of Resin-Orn(NHBoc)-Sar-Thr-NH₂ (112)

Compound III was agitated in 20% piperidine in N-methylpyrrolidine (110 mL) for 30 minutes. The reaction mixture was filtered through a glass sinter funnel, re-suspended in 20% piperidine in N-methylpyrrolidine (110 mL) and agitated for 30 minutes. The reaction mixture was filtered through a glass sinter funnel then the solid was washed with N-methylpyrrolidine (3×110 mL), methanol (3×110 mL), and again with N-methylpyrrolidine (3×110 mL) to give compound 112.

### Reaction 7 Preparation of Resin-Orn(HBoc)-Sar-Thr-Asp(OtBu)-NHFmoc (113)

Commercially available Nα-(9-Fluorenylmethoxycarbonyl)-L-aspartic acid β-tert-butyl ester, TBTU (2.7 g), HOBt (1.13 g) as a solution in N-methylpyrrolidone (100 mL) was added to compound 112 (11 g) followed by addition of diisopropylethylamine (DIPEA, 2.9 mL). The mixture was shaken for 60 minutes, filtered (through a glass sinter funnel and a few beads were tested for the presence of a free amine using the standard Kaiser test (vide supra). The Kaiser test gave a yellow color so the coupling was deemed complete. After filtration v the product bearing resin was washed with N-methylpyrrolidine (3×115 mL), methanol (3×115 mL), and again with N-methylpyrrolidine (3×115 mL) to give 113.

### Reaction 8

### Preparation of Resin-Orn(NHBoc)-Sar-Thr-Asp(OtBu)-NH₂ (114)

Compound 113.was agitated in 20% piperidine in N-methylpyrrolidine (115 mL) for 30 minutes. The reaction mixture was filtered through a glass sinter funnel, re-suspended in 20% piperidine in N-methylpyrrolidine (115 mL) and agitated for 30 minutes. The reaction mixture was filtered through a glass sinter funnel then the solid was washed with N-methylpyrrolidine (3×120 mL), methanol (3×120 mL), and again with N-methylpyrrolidine (3×120 mL) to give compound 114.

### Reaction 9

### Preparation of Resin-Orn(NHBoc)-Sar-Thr-Asp(OtBu)-DAsn HTrt)-NHFmoc (115)

Commercially available Nα-(9-Fluorenylmethoxycarbonyl)-D-asparagine (5.0 g), 2-(1H-Benzotriazol-yl)-1,1,3,3-tetramethyluronium tetrafluoroborate (TBTU, 2.7 g), HOBt (1.13 g) and diisopropylethylamine (DIPEA, 2.9 mL) as a solution in NMP (120 mL) was added to compound 114 (12 g). The mixture was shaken for 60 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×125 mL), methanol (3×125 mL), and again with N-methylpyrrolidine (3×125 mL) to give compound 115.

### Reaction 10

### Preparation of Resin-Orn(NHBoc)-Sar-Thr-Asp(OtBu)-DAsn(NHTrt)-NH₉ (116)

Compound 115 was agitated in 20% piperidine in N-methylpyrrolidine (130 mL) for 30 minutes. The reaction mixture was filtered through a glass sinter funnel, re-suspended in 20% piperidine in N-methylpyrrolidine (130 mL) and agitated for 30 minutes. The reaction mixture was filtered through a glass sinter funnel then the solid was washed with N-methylpyrrolidine (3×130 mL), methanol (3×130 mL), and again with N-methylpyrrolidine (3×130 mL) to give compound 116.

### Reaction 11

### Preparation of Resin-Orn(NHBoc)-Sar-Thr-Asp(OtBu)-DAsn(NHTrt)-Trp-NHFmoc (117)

Commercially available Nα-(9-Fluorenylmethoxycarbonyl)-L-tryptophan (3.57 g), 2-(1H-Benzotriazol-yl)-1,1,3,3-tetramethyluronium tetrafluoroborate (TBTU, 2.7 g), HOBt (1.13 g) and diisopropylethylamine (DIPEA, 2.9 mL) as a solution in N-methylpyrrolidone (130 mL) was added to compound 116 (13 g). The mixture was shaken for 60 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×135 mL), methanol (3×135 mL), and again with N-methylpyrrolidine (3×135 mL) to give compound 117.

### Reaction 12

### Preparation of Resin-Orn(NHBoc)-Sar-Thr-Asp(OtBu)-DAsn(NHTrt)-Trp-NH₂ (118)

Compound 117 was agitated in 20% piperidine in N-methylpyrrolidine (130 mL) for 30 minutes. The reaction mixture was filtered through a glass sinter funnel, re-suspended in 20% piperidine in N-methylpyrrolidine (130 mL) and agitated for 30 minutes. The reaction mixture was filtered through a glass sinter funnel then the solid was washed with N-methylpyrrolidine (3×130 mL), methanol (3×130 mL), and again with N-methylpyrrolidine (3×130 mL) to give compound 118.

### Reaction 13

### Preparation of Resin-Orn(NHBoc)-Sar-Thr-Asp(OtBu)-DAsn(NHTrt-Try-8-Methyldecanoic amide (105)

Commercially available 8-methyldecanoic acid (1.56 g), 2-(1H-Benzotriazol-yl)-1,1,3,3-tetramethyluronium tetrafluoroborate (TBTU, 2.7 g), diisopropylethylamine (DIPEA, 2.9 mL), and 1-hydroxy-benzotriazole (1.25 g) as a solution in N-methylpyrrolidine (120 mL) was added to compound 118 (13.8 g). The mixture was shaken for 18 hours, filtered through a glass sinter funnel, and the reaction was judged to be complete using the Kaiser Test (vide supra). The reaction mixture was filtered through a glass sinter funnel then the solid was washed with N-methylpyrrolidine (3×120 mL), methanol (3×120 mL), and again with N-methylpyrrolidine (3×120 mL) to give compound 105.

### EXAMPLE 1-25

### Synthesis of Peptide Resin Compound 119 Resin-Orn(NHBoc)-Sar-Thr-Asp(OtBu)-DGlu(OtBu)-Trp-8-Methyldecanoic amide (119)

### Reaction 1

### Preparation of Resin-Orn(NHBoc)-Sar-Thr-Asp(OtBu)-DGlu(OtBu)-NHFmoc. (120)

Commercially available Nα-(9-Fluorenylmethoxycarbonyl)-D-glutamic acid γ-t-butyl ester (2.29 g), TBTU (1.73 g), HOBt (0.73 g) and DIPEA (1.9 mL) as a solution in NMP (25 mL) were added to compound 114 (3.3 g). The mixture was shaken for three hours. The reaction mixture was filtered through a glass sinter funnel then the solid was washed with N-methylpyrrolidine (3×25 mL), methanol (3×25 mL), and again with N-methylpyrrolidine (3×25 mL) to give compound 120.

### Reaction 2

### Preparation of Resin-Orn(NHBoc)-Sar-Thr-Asp(OtBu)-DGlu(OtBu)-NH₂ (121)

Compound 120 was agitated in 20% piperidine in N-methylpyrrolidine (25 mL) for 30 minutes. The reaction mixture was filtered through a glass sinter funnel, re-suspended in 20% piperidine in N-methylpyrrolidine (25 mL) and agitated for 30 minutes. The reaction mixture was filtered through a glass sinter funnel then the solid was washed with N-methylpyrrolidine (3×30 mL), methanol (3×30 ml), and again with N-methylpyrrolidine (3×30 mL) to give compound 121.

### Reaction 3

### Preparation of Resin-Orn(NHBoc)-Sar-Thr-Asp(OtBu)-DGlu(OtBu)-Trp-NHFmoc (122)

Commercially available Nα-(9-Fluorenylmethoxycarbonyl)-L-tryptophan (2.30 g), 2-(1H-Benzotriazol-yl)-1,1,3,3-tetramethyluronium tetrafluoroborate (TBTU, 1.7 g), HOBt (0.73 g) and diisopropylethylamine (DIPEA, 1.9 mL) as a solution in N-methylpyrrolidone (25 mL) was added to compound 121 (3.5 g). The mixture was shaken for 60 minutes, filtered through a glass sinter funnel and a few beads were tested for the presence of a free amine using the standard Kaiser test (vide supra). The Kaiser test gave a yellow color so the coupling was deemed complete. After filtration through a glass sinter funnel the product bearing resin was washed with N-methylpyrrolidine (3×25 mL), methanol (3×25 mL), and again with N-methylpyrrolidine (3×25 mL) to give compound 122.

### Reaction 4

### Preparation of Resin-Orn(NHBoc)-Sar-Thr-Asp(OtBu)-DGlu(OtBu)-Trp-NH₂ (123)

Compound 122 was agitated in 20% piperidine in N-methylpyrrolidine (25 mL) for 30 minutes. The reaction mixture was filtered through a glass sinter funnel, re-suspended in 20% piperidine in N-methylpyrrolidine (25 mL) and agitated for 30 minutes. The reaction mixture was filtered through a glass sinter funnel then the solid was washed with N-methylpyrrolidine (3×30 mL), methanol (3×30 mL), and again with N-methylpyrrolidine (3×30 mL) to give compound 123.

### Reaction 5

### Preparation of Resin-Orn(NHBoc)-Sar-Thr-Asp(OtBu)-DGlu(OtBu)-Trp-8-Methyldecanoic amide (119)

Commercially available 8-methyldecanoic acid (0.50 g), 2-(1H-Benzotriazol-yl)-1,1,3,3-tetramethyluronium tetrafluoroborate (TBTU, 0.86 g), diisopropylethylamine (DIPEA, 0.94 mL), and 1-hydroxy-benzotriazole (0.35 g) as a solution in N-methylpyrrolidine (30 mL) was added to compound 123 (3.8 g). The mixture was shaken for 18 hours, filtered through a glass sinter funnel, and the reaction was judged to be complete using the Kaiser Test (vide supra). The reaction mixture was filtered through a glass sinter funnel then the solid was washed with N-methylpyrrolidine (3×36 mL), methanol (3×36 mL), and again with N-methylpyrrolidine (3×36 mL) to give compound 119.

### EXAMPLE 1-26

### Esterification and Cleavage of peptide Resin Compound 76

### Gly-Thr(OIleNHFmoc)-Asp(OtBu)-DAsn(NHTrt)-Trp-8-Methyldecanoic amide (126)

Commercially available Nα-(9-Fluorenylmethoxycarbonyl)-L-isoleucine (1.7 g), 4-dimethylaminopyridine (117 mg), and N-methyl-2-chloropyridinium iodide (1.23 g) were flushed well with argon then suspended in dichloromethane (20 mL). Triethylamine (76 μL) was added and the reaction mixture was stirred to give a homogeneous solution. Compound 76 (2.0 g) was added to the solution. The flask was flushed again with argon and then shaken for 14 hours. The resulting resin was then filtered through a glass sinter funnel and washed well with dichloromethane. The solid was suspended in dichloromethane (21 mL), 2,2,2-trifluoroethanol (7 mL), and acetic acid (7 mL), and shaken for 3 hours. The resin was filtered through a glass sinter funnel and evaporation of the filtrate gave the desired peptide 126 (285 mg) as a white solid.

### EXAMPLE 1-27

### Esterification and Cleavage of peptide Resin Compound 77

### Preparation of Gly-Thr(OIleNHFmoc)-Asp(OtBu)-DAsn(NHTrt)-Trp-Tridecanoic Amide (127)

Commercially available Nα-(9-Fluorenylmethoxycarbonyl)-L-isoleucine (1.48 g), 4-dimethylaminopyridine (102 mg), and N-methyl-2-chloropyridinium iodide (1.07 g) were flushed well with argon then suspended in dichloromethane (20 mL). Triethylamine (1.17 mL) was added and the reaction mixture was stirred to give a homogeneous solution. Compound 77 (1.75 g) was added to the solution. The flask was flushed again with argon and then shaken for 14 hours. The resulting resin was then filtered through a glass sinter funnel and washed well with dichloromethane. The solid was suspended in dichloromethane (15 mL), 2,2,2-trifluoroethanol (5 mL), and acetic acid (5 mL), and shaken for 4 hours. The resin was filtered through a glass sinter funnel and evaporation of the filtrate gave the desired peptide 127 (490 mg) as a white solid.

### EXAMPLE 1-28

### Esterification and Cleavage of Peptide Resin Compound 78

### Preparation of Gly-Thr(OIleNHFmoc)-Asp(OtBu)-DGlu(OtBu)-Trp-8-Methyldecanoic amide (128)

To compound 78 (5.9 g) was added a solution of commercially available Nα-(9-Fluorenylmethoxycarbonyl)-L-isoleucine (4.9 g), bromo-tris-pyrrolidinophosphonium hexafluorophosphate (PyBrOP, 6.5 g), and di-isopropylethylamine (7.3 mL), in dichloromethane (60 mL). Dimethylaminopyridine (25 mg) was added and the mixture was shaken for 2 hours. After 2 h, the mixture was filtered through a glass sinter funnel and washed with dichloromethane (3×60 mL) and the coupling procedure was repeated. The resulting resin was filtered through a glass sinter funnel, washed with dichloromethane (3×60 mL) and methanol (3×60 mL), and dried under diminished pressure over potassium hydroxide pellets. This dried resin was suspended in dichloromethane (27 mL), 2,2,2-trifluoroethanol (9 mL), and acetic acid (9 mL), and shaken for 3 hours. The resin was filtered through a glass sinter funnel and evaporation of the filtrate gave the desired peptide 128 (2.1 g) as a white solid.

### EXAMPLE 1-29

### Esterification and Cleavage of Peptide Resin Compound 83

### Preparation of Ala-Sar-Thr(OIleNHFmoc)-Asp(OtBu)-DAsn(ONHTrt)-Trp-8-Methyldecanoic amide (129)

To compound 83 (3.3 g) was added a solution of commercially available Nα-(9-Fluorenylmethoxycarbonyl)-L-isoleucine (3.2 g), bromo-tris-pyrrolidinophosphonium hexafluorophosphate (PyBrOP, 4.2 g), and Di-isopropylethylamine (4.7 mL), in dichloromethane (60 mL). Dimethylaminopyridine (23 mg), was added and the mixture was shaken for 2 hours. After 2 h, the mixture was filtered through a glass sinter funnel and washed with dichloromethane (3×30 mL) and the coupling procedure was repeated. The resulting resin was filtered through a glass sinter funnel, washed with dichloromethane (3×30 mL) and methanol (3×30 mL), and dried under diminished pressure over potassium hydroxide pellets. This dried resin was suspended in dichloromethane (24 mL), 2,2,2-trifluoroethanol (6 mL), and acetic acid (6 mL), and shaken for 3 hours. The resin was filtered through a glass sinter funnel and evaporation of the filtrate gave the desired peptide 129 (2.9 g) as a white solid.

### EXAMPLE 1-30

### Esterification and Cleavage of peptide Resin Compound 75

### Preparation of Ala-Sar-Thr(OIleNHAlloc)-Asp(OtBu)-DAsn(NHTrt)-Trp-Undecanoic amide (130)

Nα-(Allyloxycarbonyl)-L-isoleucine 124 (1.34 g, vide infra), 4-dimethylaminopyridine (15 mg), and N-methyl-2-chloropyridinium iodide (1.59 g) were flushed well with argon then suspended in dichloromethane (30 mL). Triethylamine (1.74 mL) was added and the reaction mixture was stirred to give a homogeneous solution. Compound 75 (1.25 g) was added to the solution. The flask was flushed again with argon and then shaken for 14 hours. The resulting resin was then filtered through a glass sinter funnel and washed well with dichloromethane. The solid was suspended in dichloromethane (12 mL), 2,2,2-trifluoroethanol (4 mL), and acetic acid (4 mL), and shaken for 3 hours. The resin was filtered through a glass sinter funnel and evaporation of the filtrate gave the desired peptide 130 (154 mg) as a white solid.

### EXAMPLE 1-31

### Esterification and Cleavage of Peptide Resin Compound 84

### Preparation of Ala-Sar-Thr(OIleNHFmoc)-Asp(OtBu)-DGlu(OtBu)-Trp-8-Methyldecanoic amide (131)

To compound 84 (4.8 g) was added a solution of commercially available Nα-(9-fluorenylmethoxycarbonyl)-L-isoleucine (3.2 g), bromo-tris-pyrrolidinophosphonium hexafluorophosphate (PyBrOP, 4.2 g), and Di-isopropylethylamine (4.7 mL), in dichloromethane (60 mL). Dimethylaminopyridine (23 mg) was added and the mixture was shaken for 2 hours. After 2 h, the mixture was filtered through a glass sinter funnel and washed with dichloromethane (3×30 mL) and the coupling procedure was repeated. The resulting resin was filtered through a glass sinter funnel, washed with dichloromethane (3×30 mL) and methanol (3×30 mL), and dried under diminished pressure over potassium hydroxide pellets. This dried resin was suspended in dichloromethane (24 mL), 2,2,2-trifluoroethanol (6 mL), and acetic acid (6 mL), and shaken for 3 hours. The resin was filtered through a glass sinter funnel and evaporation of the filtrate gave the desired peptide 131 (2.92 g) as a white solid.

### EXAMPLE 1-32

### Esterification and Cleavage of Peptide Resin Compound 89

### Preparation of Ala-Gly-Thr(OIleNHAlloc)-Asp(OtBu)-DAsn(NHTrt)-Trp-Undecanoic amide (132)

Nα-(Allyloxycarbonyl)-L-isoleucine 124 (1.34 g, vide infra), 4-dimethylaminopyridine (15 mg), and N-methyl-2-chloropyridinium iodide (1.59 g) were flushed well with argon then suspended in dichloromethane (30 mL). Triethylamine (1.74 mL) was added and the reaction mixture was stirred to give a homogeneous solution. Compound 89 (1.25 g) was added to the solution. The flask was flushed again with argon and then shaken for 14 hours. The resulting resin was then filtered through a glass sinter funnel and washed well with dichloromethane. The solid was suspended in dichloromethane (12 mL), 2,2,2-trifluoroethanol (4 mL), and acetic acid (4 mL), and shaken for 3 hours. The resin was filtered through a glass sinter funnel and evaporation of the filtrate gave the desired peptide 132 (147 mg) as a white solid.

### EXAMPLE 1-33

### Esterification and Cleavage of Peptide Resin Compound 100

### Preparation of Ala-Gly-Thr(OIleNHAlloc)-Asp(OtBu)-DGlu(OtBu)-Trp-Undecanoic amide (133)

Nα-(Allyloxycarbonyl)-L-isoleucine 124 (1.34 g, vide infra), 4-dimethylaminopyridine (15 mg), and N-methyl-2-chloropyridinium iodide (1.59 g) were flushed well with argon then suspended in dichloromethane (30 mL). Triethylamine (1.74 mL) was added and the reaction mixture was stirred to give a homogeneous solution. Compound 100 (1.25 g) was added to the solution. The flask was flushed again with argon and then shaken for 14 hours. The resulting resin was then filtered through a glass sinter funnel and washed well with dichloromethane. The solid was suspended in dichloromethane (12 mL), 2,2,2-trifluoroethanol (4 mL), and acetic acid (4 mL), and shaken for 3 hours. The resin was filtered through a glass sinter funnel and evaporation of the filtrate gave the desired peptide 133 (95 mg) as a white solid.

### EXAMPLE 1-34

### Esterification and Cleavage of Peptide Resin Compound 105

### Preparation of Orn(NHBoc)-Sar-Thr(OIleNHFmoc)-Asp(OtBu)-DAsn(NHTrt)-Trp-8-Methyldecanoic amide (134)

Commercially available Nα-(9-Fluorenylmethoxycarbonyl)-L-isoleucine (2.0 g), 4-dimethylaminopyridine (140 mg), and N-methyl-2-chloropyridinium iodide (1.46 g) were flushed well with argon then suspended in dichloromethane (20 mL). Triethylamine (1.6 mL) was added and the reaction mixture was stirred to give a homogeneous solution. Compound 105 (2.0 g) was added to the solution. The flask was flushed again with argon and then shaken for 14 hours. The resulting resin was then filtered through a glass sinter funnel and washed well with dichloromethane. The solid was suspended in dichloromethane (21 mL), 2,2,2-trifluoroethanol (7 mL), and acetic acid (7 mL), and shaken for 3 hours. The resin was filtered through a glass sinter funnel and evaporation of the filtrate gave the desired peptide 134 (890 mg) as a white solid.

### EXAMPLE 1-35

### Esterification and Cleavage of Peptide Resin Compound 119

### Preparation of Orn(NHBoc)-Sar-Thr(OIleNHFmoc)-Asp(OtBu)-DGlu(OtBu)-Trp-8-Methyldecanoic amide (135)

Commercially available Nα-(9-Fluorenylmethoxycarbonyl)-L-isoleucine (2.0 g), 4-dimethylaminopyridine (140 mg), and N-methyl-2-chloropyridinium iodide (1.46 g) were flushed well with argon then suspended in dichloromethane (20 mL). Triethylamine (1.6 mL) was added and the reaction mixture was stirred to give a homogeneous solution. Compound 119 (1.75 g) was added to the solution. The flask was flushed again with argon and then shaken for 14 hours. The resulting resin was then filtered through a glass sinter funnel and washed well with dichloromethane. The solid was suspended in dichloromethane (21 mL), 2,2,2-trifluoroethanol (7 mL), and acetic acid (7 mL), and shaken for 3 hours. The resin was filtered through a glass sinter funnel and evaporation of the filtrate gave the desired peptide 135 (761 mg) as a white solid.

### EXAMPLE 1-36

### Preparation of Compound C16

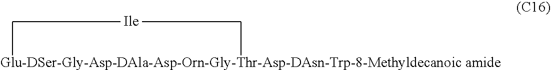

### Reaction 1

### Preparation of Resin-Glu((αOAllyl)-DSer(OtBu)-Gly-Asp(OtBu)-DAla-Asp(OtBu)-Orn(NHBoc)-Gly-Thr(OIleNHFmoc)-Asp(OtBu)-DAsn(NHTrt)-Trp-8-Methyldecanoic amide (137)

Hydroxy-benzotriazole (17 mg), benzotriazole-1-yl-oxy-tris-(dimethylamino)-phosphoniumhexafluorophosphonate (BOP, 55 mg), and diisopropylethylamine (22 μL), were added to a solution of compound 126 (174 mg) in dimethylformamide (3 mL), then compound 9 (300 mg) was added and the mixture then shaken for 17 hours. A portion of the resin was removed and the coupling was judged to be complete using the Kaiser Test (vide supra). The resin was filtered through a glass sinter funnel, washed with dichloromethane (3×3 mL) and dried under reduced pressure, yielding resin bound compound 137.

Reaction 2: Preparation of

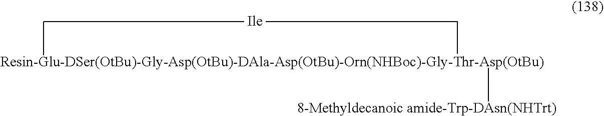

Compound 137 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) and dried under reduced pressure. The resin was washed with DMF:piperidine 4:1 (10 mL) for 4 hrs then filtered through a glass sinter funnel. The solid was washed with dimethylformamide (10 mL), and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 138.

Reaction 3: Preparation of compound (C16)

Dried compound 138 was suspended in dichloromethane, (2.5 mL) trifluoroacetic acid, (2.5 mL) ethanedithiol (125 μL), and triisopropylsilane(125 μL), and the reaction mixture was stirred for 4.5 hours at ambient temperature. The resin was filtered through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give the pure product C16 (3.7 mg).

### EXAMPLE 1-37

### Preparation of Compound C76

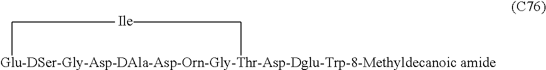

### Reaction 1

### Preparation of Resin-Glu(αOAllyl)-DSer(OtBu)-Gly-Asp(OtBu)-DAla-Asp(OtBu)-Orn(NHBoc)-Gly-Thr(OleNHFmoc)-Asp(OtBu)-DGlu(OtBu)-TrM-8-Methyldecanoic amide (140)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 128 (174 mg) in dimethylformamide (3 mL). Compound 9 (300 mg) was added and the mixture was shaken for 17 hours. A portion of the resin was removed and the coupling was judged to be complete using the Kaiser Test (vide supra). The resin was filtered through a glass sinter funnel, washed with dichloromethane (3×3 mL) and dried under reduced pressure, yielding resin bound compound 140.

Reaction 2: Preparation of

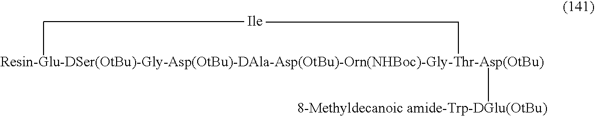

Compound 140 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hours then filtered through a glass sinter funnel. The solid was washed with dimethylformamide (10 mL), and dichloromethane (10 mL) then dried under reduced pressure. The resin was suspended in N-methylmorpholine (3 mL) then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 141.

Reaction 3: Preparation of compound (C76)

Dried compound 141 was suspended in dichloromethane, (2.5 mL) trifluoroacetic acid, (2.5 mL) ethanedithiol (125 μL), and triisopropylsilane (125 μL), and the reaction mixture was stirred for 4.5 hours at ambient temperature. The resin was filtered through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give the pure product C76 (9.0 mg).

### EXAMPLE 1-38

### Preparation of Compound C75

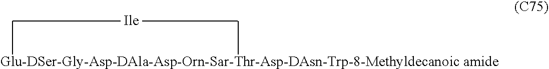

### Reaction 1

### Preparation of Resin-Gly(αOAllyl)-DSer(OtBu)-Gly-Asp(OtBu)-DAla-Asp(OtBu)-Orn(NHBoc)-Sar-Thr(OIleNHFmoc)-Asp(OtBu)-Trp-8-Methyldecanoic amide (143)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 134 (243 mg) in dimethylformamide (3 mL). Compound 21 (322 mg) was added and the mixture was shaken for 17 hours. A portion of the resin was removed and the coupling was judged to be complete using the Kaiser Test (vide supra). The resin was filtered through a glass sinter funnel, washed with dichloromethane (3×3 mL) and dried under reduced pressure, yielding compound 143.

Reaction 2: Preparation of

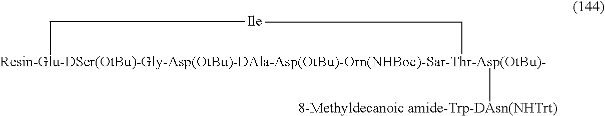

Compound 143 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel). The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 144.

Reaction 3: Preparation of compound (C75)

Dried compound 144 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered through a glass sinter funnel, washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C75 (8.1 mg).

### EXAMPLE 1-39 PREPARATION OF COMPOUND C74

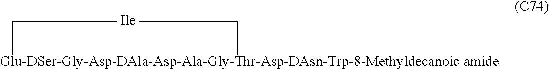

### Reaction 1

### Preparation of Resin-Glu(αOAllyl)-DSer(OtBu)-Gly-Asp(OtBu)-DAla-Asp(OtBu)-Ala-Gly-Thr(OIleNHFmoc)-Asp(OtBu)-DAsn(NHTrt)-Trp-8-Methyldecanoic amide (146)

Hydroxy-benzotriazole (27 mg), benzotriazole-1-yl-oxy-tris-(dimethylamino)-phosphoniumhexafluorophosphonate (BOP, 88.5 mg), and diisopropylethylamine (100 μL), were added to a solution of compound 126 (278 mg) in dimethylformamide (3 mL). Compound 38 (227 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 146.

### Reaction 2

### Preparation of compound (147)

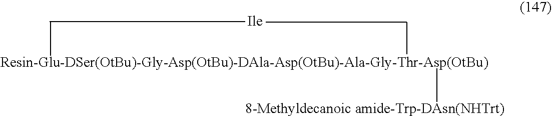

Compound 146 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 147.

### Reaction 3 Preparation of compound (C74)

Dried compound 147 was suspended in dichloromethane (2.5 mL), trifluoroacetic acid (2.5 mL), ethanedithiol (125 μL), and triisopropylsilane(125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C74 (3.9 mg).

### EXAMPLE 1-40

### Preparation of

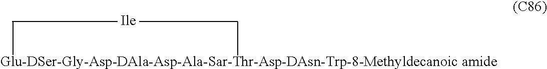

### Reaction 1

### Preparation of Resin-Glu(αOAllyl)-DSer(OtBu)-Gly-Asp(OtBu)-DAla-Asp(OtBu)-Ala-Sar-Thr(OIleNHFmoc)-Asp(OtBu)-DAsn(NHTrt)-Trp-8-Methyldecanoic amide (149)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 129 (228 mg) in dimethylformamide (3 mL). Compound 21 (280 mg) was added and the mixture was shaken for 17 hours. A portion of the resin was removed and the coupling was judged to be complete using the Kaiser Test(vide supra). The resin was filtered through a glass sinter funnel, washed with dichloromethane (3×3 mL) and dried under reduced pressure, yielding compound 149.

Reaction 2: Preparation of

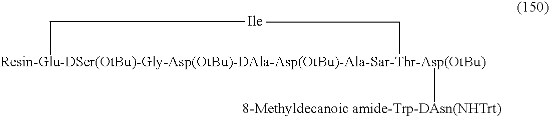

Compound 149 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel. The solid was washed with dimethylformamide (10 mL), dichloromethane (10 mL) and dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 7 hours, filtered through a glass sinter funnel, and washed well with N-methylmorpholine to give compound 150.

Reaction 3: Preparation of (C86)

Dried compound 150 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C86 (2.8 mg).

### EXAMPLE 1-41

### Preparation of Compound C79

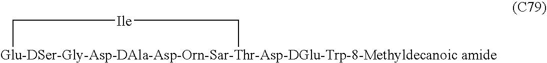

### Reaction 1

### Preparation of Resin-Glu(αOAllyl)-DSer(OtBu)-Gly-Asp(OtBu)-DAla-Asp(OtBu)-Orn(NHBoc)-Sar-Thr(OIleNHFmoc)-Asp(OtBu)-DGlu(OtBu)-Trp-8-Methyldecanoic amide (152)

Hydroxy-benzotriazole (20 mg), benzotriazole-1-yl-oxy-tris-(dimethylamino)-phosphoniumhexafluorophosphonate (BOP, 66 mg), and diisopropylethylamine (52 μL), were added to a solution of compound 135 (217 mg) in dimethylformamide (3 mL). Compound 21 (278 mg) was added and the mixture was shaken for 17 hours. A portion of the resin was removed and the coupling was judged to be complete using the Kaiser Test (vide supra). The resin was filtered through a glass sinter funnel, washed with dichloromethane (3×3 mL) and dried under reduced pressure, yielding resin bound compound 152.

Reaction 2: Preparation of

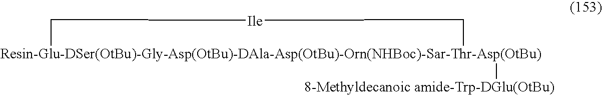

Compound 151 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel. The solid was washed with dimethylformamide (10 mL) and dichloromethane (10 mL) then dried under reduced pressure. The resin was suspended in N-methylmorpholine (3 mL) then 1-hydroxy-benzotriazole (135 mg) and 1,3-diisopropylcarbodiimide (157 μL) were added. The reaction was shaken for 7 hours, filtered through a glass sinter funnel, and washed well with N-methylmorpholine to give compound 153.

Reaction 3: Preparation of compound (C79)

Dried compound 153 was suspended in dichloromethane (2.5 mL), trifluoroacetic acid (2.5 mL), ethanedithiol (125 μL), and triisopropylsilane(125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C79 (1.5 mg).

### EXAMPLE 1-42

### Preparation of Compound C81

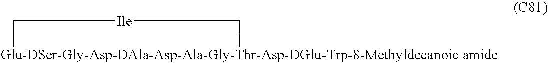

### Reaction 1

### Preparation of Resin-Glu(αOAllyl)-DSer(OtBu)-Gly-Asp(OtBu)-DAla-Asp(OtBu)-Ala-Gly-Thr(OIleNHFmoc)-Asp(OtBu)-DGlu(OtBu)-TrM-8-Methyldecanoic amide (155)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 128 (183 mg) in dimethylformamide (3 mL). Compound 38 (227 mg) was added and the mixture was shaken for 17 hours. A portion of the resin was removed and the coupling was judged to be complete using the Kaiser Test (vide supra). The resin was filtered through a glass sinter funnel, washed with dichloromethane (3×3 mL) and dried reduced pressure, yielding compound 155.

Reaction 2: Preparation of

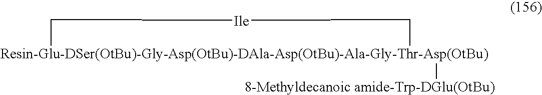

Compound 155 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 156.

Reaction 3: Preparation of (C81)

Dried compound 156 was suspended in dichloromethane (2.5 mL), trifluoroacetic acid (2.5 mL), ethanedithiol (125 μL), and triisopropylsilane(125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C81 (2.3 mg).

### EXAMPLE 1-43

### Preparation of Compound C80

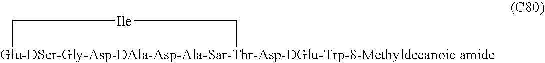

### Reaction 1

### Preparation of Resin-Glu(αOAllyl)-DSer(OtBu)-Gly-Asp(OtBu)-DAla-Asp(OtBu)-Ala-Sar-Thr(OIleNHFmoc)-Asp(OtBu)-DGlu(OtBu)-Trp-8-Methyldecanoic amide (158)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 131 (196 mg) in dimethylformamide (3 mL). Compound 21 (278 mg) was added and the mixture was shaken for 17 hours. A portion of the resin was removed and the coupling was judged to be complete using the Kaiser Test (vide supra) The resin was filtered through a glass sinter funnel, washed with dichloromethane (3×3 mL) and dried under reduced pressure, yielding compound 158.

Reaction 2: Preparation of

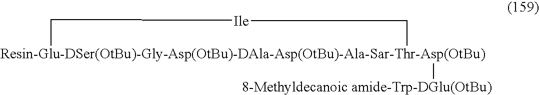

The resin 158 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 159.

Reaction 3: Preparation of compound (C80)

Dried compound 159 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C80 (6.2 mg).

### EXAMPLE 1-44

### Preparation of Compound C72

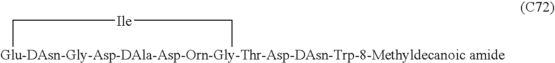

### Reaction 1

### Preparation of Resin-Glu(αOAllyl)-DAsn-Gly-Asp(OtBu)-DAla-Asp(OtBu)-Orn(NHBoc)-Gly-Thr(OIleNHFmoc)-Asp(OtBu)-DAsn(NHTrt)-Trp-8-Methyldecanoic amide (161)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 126 (274 mg) in dimethylformamide (3 mL). Compound 50 (303 mg) was added and the mixture then shaken for 17 hours. A portion of the resin was removed and the coupling was judged to be complete using the Kaiser Test (vide supra). The resin was filtered through a glass sinter funnel, washed with dichloromethane (3×3 mL) and dried under reduced pressure, yielding compound 161.

Reaction 2: Preparation of

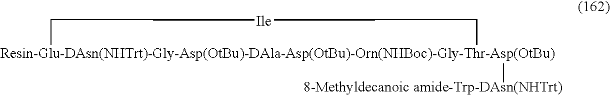

Compound 161 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel. The solid was washed with dimethylformamide (10 mL), dichloromethane (10 mL) and dried under reduced pressure. The resin was suspended in N-methylmorpholine (3 mL) and 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 162.

Reaction 3: Preparation of (C72)

Dried compound 162 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C72 (2.9 mg).

### EXAMPLE 1-45

### Preparation of C352

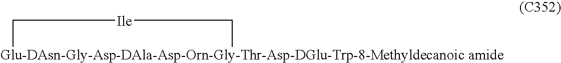

### Reaction 1

### Preparation of Resin-Glu(αOAllyl)-DAsn(NHTrt)-Gly-Asp(OtBu)-DAla-Asp(OtBu)-Orn(NHBoc)-Gly-Thr(OIleNHFmoc)-Asp(OtBu)-DGlu(OtBu)-Trp-8-Methyldecanoic amide (164)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 127 (183 mg) in dimethylformamide (2 mL). Compound 50 (265 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 164.

Reaction 2: Preparation of

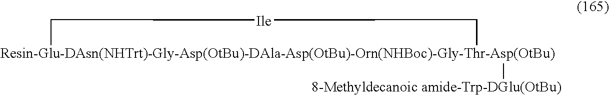

Compound 164 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 165.

Reaction 3: Preparation of compound (C352)

Dried compound 165 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C352 (4.7 mg).

### EXAMPLE 1-46

### Preparation of Compound C85

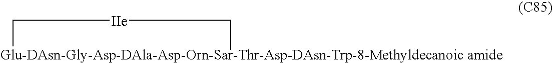

### Reaction 1

### Preparation of Resin-Glu(αOAllyl)-DAsn(NHTrt)-Gly-Asp(OtBu)-DAla-Asp(OtBu)-Orn(Boc)-Sar-Thr(OIleNHFmoc)-Asp(OtBu)-DAsn(NHTrt)-Trp-8-Methyldecanoic amide (167)

Hydroxy-benzotriazole (20 mg), benzotriazole-1-yl-oxy-tris-(dimethylamino)-phosphoniumhexafluorophosphonate (BOP, 66 mg), and diisopropylethylamine (30 μL), were added to a solution of compound 134 (248 mg) in dimethylformamide (3 mL). Compound 40 (238 mg) was added and the mixture was shaken for 17 hours. A portion of the resin was removed and the coupling was judged to be complete using the Kaiser Test(vide supra). The resin was filtered through a glass sinter funnel, washed with dichloromethane (3×3 mL) and dried under reduced pressure, yielding compound 167.

Reaction 2: Preparation of

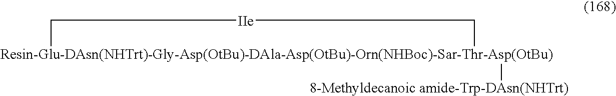

Compound 167 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel. The solid was washed with dimethylformamide (10 mL) and dichloromethane (10 mL) then dried under reduced pressure. The resin was suspended in N-methylmorpholine (3 mL) then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 168.

Reaction 3: Preparation of compound (C85)

Dried compound 168 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C85 (3.7 mg).

### EXAMPLE 1-47

### Preparation of Compound C353

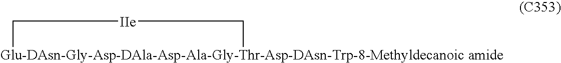

### Reaction 1

### Preparation of Resin-Glu(αOAllyl)-DAsn(NHTrt)-Gly-Asp(OtBu)-DAla-Asp(OtBu)-Ala-Gly-Thr(OIleNHFmoc)-Asp(OtBu)-DAsn(NHTrt)-Trp-8-Methyldecanoic amide (170)

Hydroxy-benzotriazole (20 mg), benzotriazole-1-yl-oxy-tris-(dimethylamino)-phosphoniumhexafluorophosphonate (BOP, 66 mg), and diisopropylethylamine (52 μL), were added to a solution of compound 126 (209 mg) in dimethylformamide (2 mL). Compound 52 (340 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 170.

Reaction 2: Preparation of

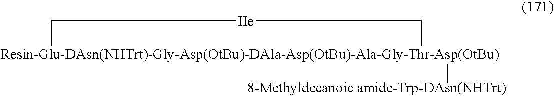

The resin 170 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel. The solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 then filtered through a glass sinter funnel. The solid was washed with dimethylformamide (10 mL) and dichloromethane (10 mL) then dried under reduced pressure. The resin was suspended in N-methylmorpholine (3 mL) then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 171.

Reaction 3: Preparation of compound (C353)

Dried compound 171 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C353 (6.8 mg).

### EXAMPLE 1-48

### Preparation of Compound C82

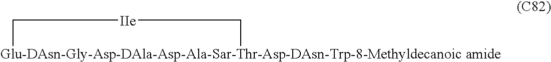

### Reaction 1

### Preparation of Resin-Glu(αOAllyl)-DAsn(NHTrt)-Gly-Asp(OtBu)-DAla-Asp(OtBu)-Ala-Sar-Thr(OIleNHFmoc)-Asp(OtBu)-DAsn(NHTrt)-Trp-8-Methyldecanoic amide (173)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 129 (221 mg) in dimethylformamide (3 mL). Compound 40 (238 mg) was added and the mixture was shaken for 17 hours. A portion of the resin was removed and the coupling was judged to be complete using the Kaiser Test (vide supra). The resin was filtered through a glass sinter funnel, washed with dichloromethane (3×3 mL) then dried under reduced pressure, yielding compound 173.

Reaction 2: Preparation of

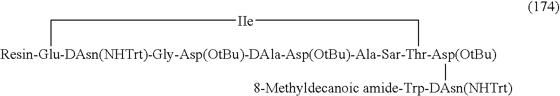

Compound 173 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered, through a glass sinter funnel, and washed well with N-methylmorpholine to give compound 174.

Reaction 3: Preparation (C82)

Dried compound 174 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C82 (3.8 mg).

### EXAMPLE 1-49

### Preparation of Compound C83

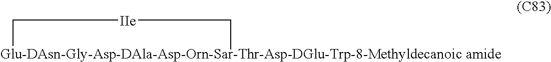

### Reaction 1

### Preparation of Resin-Glu(αOAllyl)-DAsn(NHTrt)-Gly-Asp(OtBu)-DAla-Asp(OtBu)-Orn(NHBoc)-Sar-Thr(OIleNHFmoc)-Asp(OtBu)-DGlu(OtBu)-Trp-8-Methyldecanoic amide (176)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 135 (221 mg) in dimethylformamide (3 mL). Compound 40 (238 mg) was added and the mixture was shaken for 17 hours. A portion of the resin was removed and the coupling was judged to be complete using the Kaiser Test(vide supra. The resin was filtered, through a glass sinter funnel, washed with dichloromethane (3×3 mL) and dried under reduced pressure, yielding compound 176.

Reaction 2: Preparation of

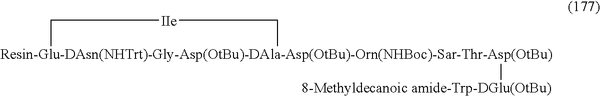

Compound 176 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 ml) for 4 hrs then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 177.

Reaction 3: Preparation of Compound (C83)

Dried compound 177 was suspended in dichloromethane (2.5 mL), trifluoroacetic acid (2.5 mL), ethanedithiol (125 μL), and triisopropylsilane(125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C83 (4.3 mg).

### EXAMPLE 1-50

### Preparation of Compound C84

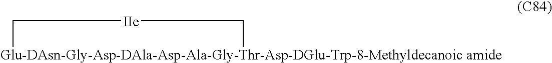

### Reaction 1

### Preparation of Resin-Glu(αOAllyl)-DAsn(NHTrt)-Gly-Asp(OtBu)-DAla-Asp(OtBu)-Ala-Gly-Thr(OIleNHFmoc)-Asp(OtBu)-DGlu(OtBu)-Trp-8-Methyldecanoic amide (179)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 128 (183 mg) in dimethylformamide (3 mL). Compound 52 (212 mg) was added and the mixture was shaken for 17 hours. A portion of the resin was removed and the coupling was judged to be complete using the Kaiser Test (vide supra). The resin was filtered through a glass sinter funnel, washed with dichloromethane (3×3 mL) and dried under reduced pressure, yielding compound 179.

Reaction 2: Preparation of

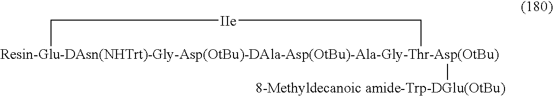

Compound 179 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF: piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 180.

Reaction 3: Preparation of compound (C84)

Dried compound 180 was suspended in dichloromethane (2.5 mL), trifluoroacetic acid (2.5 mL), ethanedithiol (125 μL), and triisopropylsilane (125 mL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C84 (6.6 mg).

### EXAMPLE 1-51

### Preparation of Compound C354

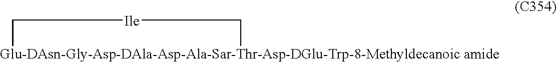

### Reaction 1

### Preparation of Resin-Glu(αOAllyl)-DAsn(NHTrt)-Gly-Asp(OtBu)-DAla-Asp(OtBu)-Ala-Sar-Thr(OIleNHFmoc)-Asp(OtBu)-DGlu(OtBu)-Trp-8-Methyldecanoic amide (182)

Hydroxy-benzotriazole (20 mg)₅ benzotriazole-1-yl-oxy-tris-(dimethylamino)-phosphoniumhexafluorophosphonate (BOP, 66 mg), and diisopropylethylamine (26 μL), were added to a solution of compound 131 (196 mg) in dimethylformamide (2 mL). Compound 40 (238 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 182.

Reaction 2: Preparation of

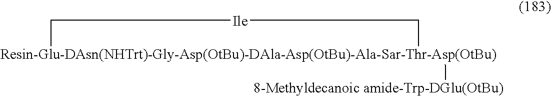

Compound 182 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hrs then filtered through a glass sinter funnel. The solid was washed with dimethylformamide (10 mL) and dichloromethane (10 mL) then dried under reduced pressure. The resin was suspended in N-methylmorpholine (3 mL) then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 183.

Reaction 3: Preparation of compound (C354)

Dried compound 183 was suspended in dichloromethane (2.5 mL), trifluoroacetic acid (2.5 mL), ethanedithiol (125 μL), and triisopropylsilane(125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C354 (4.7 mg).

### EXAMPLE 1-52

### Preparation of Compound C73

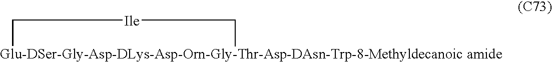

### Reaction 1

### Preparation of Resin-Glu(αOAllyl)-DSer(OtBu)-Gly-Asp(OtBu)-DLys(NHBoc)-Asp(OtBu)-Orn(NHBoc)-Gly-Thr(OIleNHFmoc)-Asp(OtBu)-DAsn(NHTrt)-Trp-8-Methyldecanoic amide (185)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 126 (298 mg) in dimethylformamide (3 mL). Compound 54 (312 mg) was added and the mixture was shaken for 17 hours. A portion of the resin was removed and the coupling was judged to be complete using the Kaiser Test (vide supra). The resin was filtered through a glass sinter funnel, washed with dichloromethane (3×3 mL) and dried under reduced pressure, yielding compound 185.

Reaction 2: Preparation of

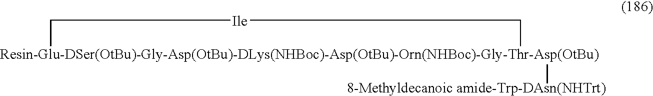

Compound 185 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 186.

Reaction 3: Preparation of compound (C73)

Dried compound 186 was suspended in dichloromethane (2.5 mL), trifluoroacetic acid (2.5 mL), ethanedithiol (125 mL), and triisopropylsilane(125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C73 (24.6 mg).

### EXAMPLE 1-53

### Preparation of Compound C355

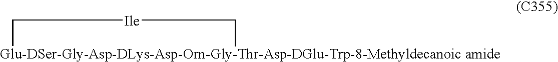

### Reaction 1

### Preparation of Resin-Glu(αOAllyl)-DSer(OtBu)-Gly-Asp(OtBu)-DLys(NHBoc)-Asp(OtBu)-Orn(NHBoc)-Gly-Thr(OIeNHFmoc)-Asp(OtBu)-DGlu(OtBu)u-Trp-8-Methyldecanoic amide (188)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 128 (183 mg) in dimethylformamide (2 mL). Compound 54 (322.6 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel), washed with dichloromethane (3×5 mL) and dried under reduced pressure, yielding compound 188.

Reaction 2: Preparation of

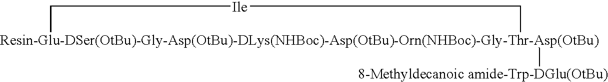

Compound 188 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 189.

Reaction 3: Preparation of (C355)

Dried compound 189 was suspended in dichloromethane (2.5 mL), trifluoroacetic acid (2.5 mL), ethanedithiol (125 μl), and triisopropylsilane (125 μl), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C355 (8.2 mg).

### EXAMPLE 1-54

### Preparation of Compound C356

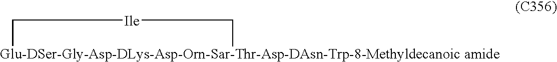

### Reaction 1

### Preparation of Resin-Glu(αOAllyl)-DSer(OtBu)-Gly-Asp(OtBu)-DLys(NHBoc)-Asp(OtBu)-Orn(NHBoc)-Sar-Thr(OIleNHFmoc)-Asp(OtBu)-DAsn(NHTrt)-Trp-8-Methyldecanoic amide (191)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 134 (243 mg) in dimethylformamide (2 mL). Compound 34 (263 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 191.

Reaction 2: Preparation of

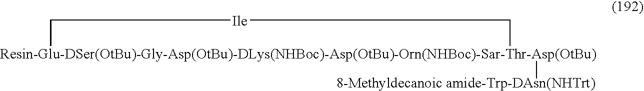

Compound 191 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 192.

Reaction 3: Preparation of (C356)

Dried compound 192 was suspended in dichloromethane (2.5 mL), trifluoroacetic acid (2.5 mL), ethanedithiol (125 μl), and triisopropylsilane(125 μl), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C356 (8.7 mg).

### EXAMPLE 1-55

### Preparation of Compound C 357

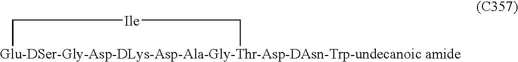

### Reaction 1

### Preparation of Resin-Glu(αOAllyl)-DSer(OtBu)-Gly-Asp(OtBu)-DLys(NHBoc)-Asp(OtBu)-Ala-Gly-Thr(OIleNHAlloc)-Asp(OtBu)-DAsn HTrt-Trp-undecanoic amide (194)

Hydroxy-benzotriazole (14 mg), benzotriazole-1-yl-oxy-tris-(dimethylamino)-phosphoniumhexafluorophosphonate (BOP, 44 mg), and diisopropylethylamine (20 μL), were added to a solution of compound 132 (147 mg) in dimethylformamide (3 mL). Compound 34 (200 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 mL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 194.

Reaction 2: Preparation of

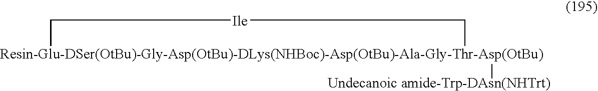

Compound 194 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 195.

Reaction 3: Preparation of (C357)

Dried compound 195 was suspended in dichloromethane (2.5 mL), trifluoroacetic acid (2.5 mL), ethanedithiol (125 μL), and triisopropylsilane(125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product (43 mg). The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C357 (4.5 mg).

### EXAMPLE 1-56

### Preparation of Compound C358

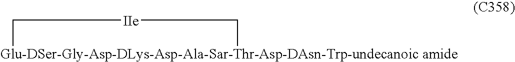

### Reaction 1

### Preparation of Resin-Glu(αOAllyl)-DSer(OtBu)-Gly-Asp(OtBu)-DLys(NHBoc)-Asp-Ala-Sar-Thr(OIleNHAlloc)-Asp(OtBu)-DAsn(NHTrt)-Trp-undecanoic amide (197)

Hydroxy-benzotriazole (14 mg), benzotriazole-1-yl-oxy-tris-(dimethylamino)-phosphoniumhexafluorophosphonate (BOP, 44 mg), and diisopropylethylamine (20 μL), were added to a solution of compound 130 (154 mg) in dimethylformamide (3 mL). Compound 34 (200 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 197.

Reaction 2: Preparation of

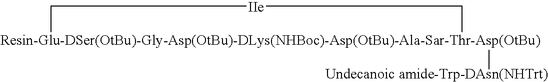

Compound 197 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 198.

Reaction 3: Preparation of compound (C358)

Dried compound 198 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel, washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C 358 (2.7 mg).

### EXAMPLE 1-57

### Preparation of Compound C359

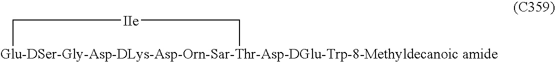

### Reaction 1

### Preparation of Resin-Glu(αOAllyl)-DSer(OtBu)-Gly-Asp(OtBu)-DLys(NHBoc)-Asp(OtBu)-Orn(NHBoc)-Sar-Thr OIeNHFmoc)-Asp(OtBu)-DGlu(OtBu)-Trp-8-Methyldecanoic amide (200)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 135 (217 mg) in dimethylformamide (2 mL). Compound 34 (263 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 200.

Reaction 2: Preparation of

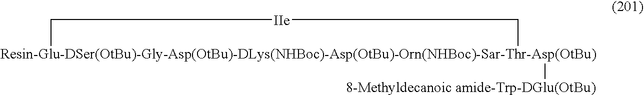

Compound 200 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 201.

Reaction 3: Preparation of (C359)

Dried compound 201 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C359 (4.7 mg).

### EXAMPLE 1-58

### Preparation of Compound C360

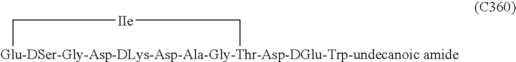

### Reaction 1

### Preparation of Resin-Glu(αOAllyl)-DSer(OtBu)-Gly-Asp(OtBu)-DLys(NHBoc)-Asp(OtBu)-Ala-Gly-Thr(OIleNHAlloc)-Asp(OtBu)-DGlu(OtBu)-Trp-undecanoic amide (203)

Hydroxy-benzotriazole (14 mg), benzotriazole-1-yl-oxy-tris-(dimethylamino)-phosphoniumhexafluorophosphonate (BOP, 44 mg), and diisopropylethylamine (20 μL), were added to a solution of compound 133 (95 mg) in dimethylformamide (3 mL). Compound 34 (200 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 203.

Reaction 2: Preparation of

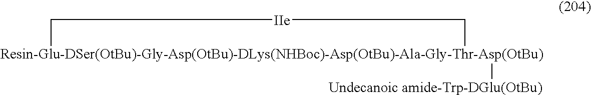

Compound 203 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 204.

Reaction 3: Preparation of compound (C360)

Dried compound 204 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C360 (3.4 mg).

### EXAMPLE 1-59

### Preparation of Compound C361

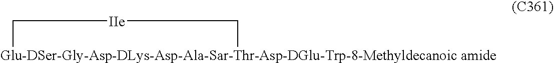

### Reaction 1

### Preparation of Resin-Glu(αOAllyl)-DSer(OtBu)-Gly-Asp(OtBu)-DLys(NHBoc)-Asp(OtBu)-Ala-Sar-Thr(OIleNHFmoc)-Asp(OtBu)-DGlu(OtBu)-Trp-8-Methyldecanoic amide (206)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 131 (196 mg) in dimethylformamide (2 mL). Compound 34 (270 mg) was added and the mixture was shaken for 17 hours. A portion of the resin was removed and the coupling was judged to be incomplete using the Kaiser Test(vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 206.

Reaction 2: Preparation of

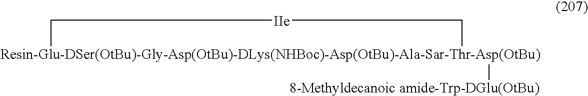

Compound 206 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF: piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 207.

Reaction 3: Preparation of compound (C361)

Dried compound 207 was suspended in dichloromethane (2.5 mL), trifluoroacetic acid (2.5 mL), ethanedithiol (125 μL), and triisopropylsilane(125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C361 (6.3 mg).

### EXAMPLE 1-60

### Preparation of Compound C77

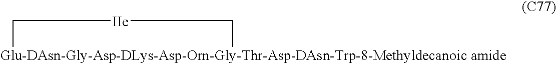

### Reaction 1

### Preparation of Resin-Glu(αOAllyl)-DAsn(NHTrt)-Gly-Asp(OtBu)-DLys(NHBoc)-Asp(OtBu)-Orn(NHBoc)-Gly-Thr(OIleNHFmoc)-Asp(OtBu)-DAsn(NHTrt)-Trp-8-Methyldecanoic amide (209)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 126 (243 mg) in dimethylformamide (3 mL). Compound 60 (217 mg) was added and the mixture was shaken for 17 hours. A portion of the resin was removed and the coupling was judged to be complete using the Kaiser Test (vide supra). The resin was filtered through a glass sinter funnel, washed with dichloromethane (3×3 mL) and dried under reduced pressure, yielding compound 209.

Reaction 2: Preparation of

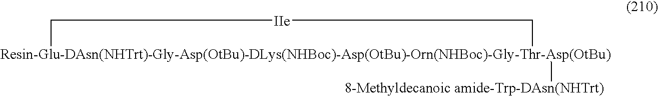

Compound 209 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 210.

Reaction 3: Preparation of compound (C77)

Dried compound 210 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C77 (3.8 mg).

### EXAMPLE 1-61

### Preparation of Compound C362

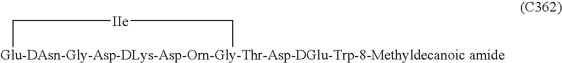

### Reaction 1

### Preparation of Resin-Glu(αOAllyl)-DAsn(NHTrt)-Gly-Asp(OtBu)-DLys(NHBoc)-Asp(OtBu)-Orn(NHBoc)-Gly-Thr(OIleNHFmoc)-Asp(OtBu)-DGlu(OtBu)-Trp-8-Methyldecanoic amide (212)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 128 (183 mg) in dimethylformamide (2 mL). Compound 60 (217 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 212.

Reaction 2: Preparation of

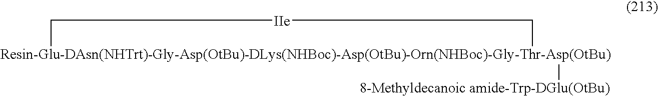

Compound 212 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 213.

Reaction 3: Preparation of compound (C362)

Dried compound 213 was suspended in dichloromethane (2.5 mL), trifluoroacetic acid (2.5 mL), ethanedithiol (125 μL), and triisopropylsilane(125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C362 (3.1 mg).

### EXAMPLE 1-62

### Preparation of Compound C363

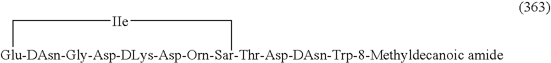

### Reaction 1

### Preparation of Resin-Glu(αOAllyl)-DAsn(NHTrt)-Gly-Asp(OtBu)-DLys(NHBoc)-Asp(OtBu)-Orn(NHBoc)-Sar-Thr(OIleNHFmoc)-Asp(OtBu)-DAsn(NHTrt)-Trp-8-Methyldecanoic amide (215)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 134 (342 mg) in dimethylformamide (2 mL). Compound 56 (416 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 mL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 215.

Reaction 2: Preparation of

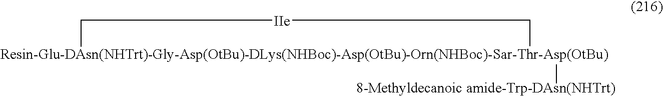

Compound 215 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 216.

Reaction 3: Preparation of compound (C363)

Dried compound 216 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C363 (6.1 mg).

### EXAMPLE 1-63

### Preparation of Compound C364

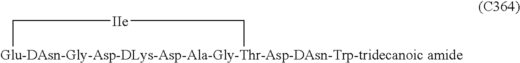

### Reaction 1

### Preparation of Resin-Glu(αOAllyl)-DAsn(NHTrt)-Gly-Asp(OtBu)-DLys(NHBoc)-Asp(OtBu)-Ala-Gly-Thr(OIleNHFmoc)-Asp(OtBu)-DAsn(NHTrt)-Trp-Tridecanoic amide (218)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 127 (146 mg) in dimethylformamide (3 mL). Compound 62 (171 mg) was added and the mixture was shaken for 17 hours. A portion of the resin was removed and the coupling was judged to be complete using the Kaiser Test (vide supra). The resin was filtered through a glass sinter funnel, washed with dichloromethane (3×3 mL) and dried under reduced pressure, yielding compound 218.

Reaction 2: Preparation of

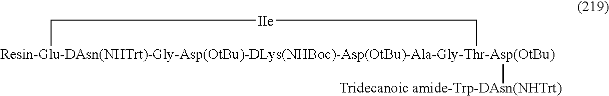

Compound 218 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF: piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 219.

Reaction 3: Preparation of compound (C364)

Dried compound 219 was suspended in dichloromethane (2.5 mL), trifluoroacetic acid (2.5 mL), ethanedithiol (125 μL), and triisopropylsilane(125 μL), and the reaction mixture was stirred for 4.5 hours at ambient temperature. The resin was filtered through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C364 (4.8 mg).

### EXAMPLE 1-64

### Preparation of Compound C365

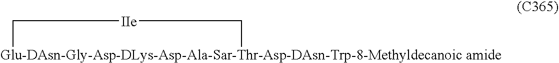

### Reaction 1

### Preparation of Resin-Glu(αOAllyl)-DAsn(NHTrt)-Gly-Asp(OtBu-DLys(NHBoc)-Asp(OtBu)-Ala-Sar-Thr(OIleNHFmoc)-Asp(OtBu)-DAsn(NHTrt)-Trp-8-Methyldecanoic amide (221)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μl), were added to a solution of compound 129 (195 mg) in dimethylformamide (2 mL). Compound 56 (400 mg) was added and the mixture was shaken for 17 hours. A portion of the resin was removed and the coupling was judged to be incomplete using the Kaiser Test (vide-supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel washed with dichloromethane (3×5 mL) and dried under reduced pressure, yielding compound 221.

Reaction 2: Preparation of

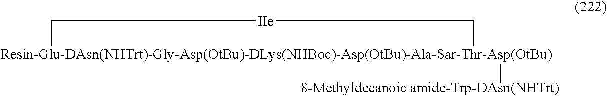

Compound 221 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF:piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 222.

Reaction 3: Preparation of compound (C365)

Dried compound 222 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C365 (10.2 mg).

### EXAMPLE 1-65

### Preparation of Compound C366

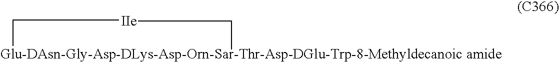

### Reaction 1

### Preparation of Resin-Glu(αOAllyl)-DAsn(NHTrt)-Gly-Asp(OtBu)-DLys(NHBoc)-Asp(OtBu)-Orn(NHBoc)-Sar-Thr(OIleNHFmoc)-Asp(OtBu)-DGlu(OtBu)-Trp-8-Methyldecanoic amide (224)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 135 (217 mg) in dimethylformamide (2 mL). Compound 56 (217 mg) was added and the mixture was shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 mL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 224.

Reaction 2: Preparation of

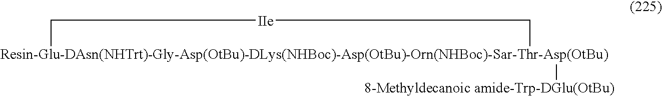

Compound 224 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF: piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 225.

Reaction 3: Preparation of Compound (C366)

Compound 225 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C366 (1.1 mg).

### EXAMPLE 1-66

### Preparation of Compound C367

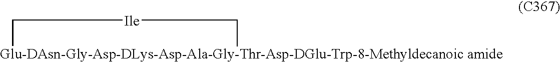

### Reaction 1

### Preparation of Resin-Glu(αOAllyl)-DAsn(NHTrt)-Gly-Asp(OtBu)-DLys(NHBoc)-Asp(OtBu)-Ala-Gly-Thr(OIleNHFmoc)-Asp(OtBu)-DGlu(OtBu)-Trp-8-Methyldecanoic amide (227)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 128 (183 mg) in dimethylformamide (3 mL). Compound 62 (294 mg) was added and the mixture was shaken for 17 hours. A portion of the resin was removed and the coupling was judged to be complete using the Kaiser Test (vide supra). The resin was filtered through a glass sinter funnel, washed with dichloromethane (3×3 mL) and dried under reduced pressure, yielding compound 227.

Reaction 2: Preparation of

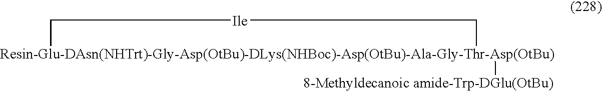

Compound 227 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF: piperidine 4:1 (10 mL) for 4 hours then filtered through a glass sinter funnel. The solid was washed with dimethylformamide (10 mL) and dichloromethane (10 mL) then dried under reduced pressure. The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered through a glass sinter funnel, and washed well with N-methylmorpholine to give compound 228.

Reaction 3: Preparation of (C367)

Dried compound 228 was suspended in dichloromethane (2.5 mL), trifluoroacetic acid (2.5 mL), ethanedithiol (125 μL), and triisopropylsilane(125 μL), and the reaction mixture was stirred for 4.5 hours at ambient temperature. The resin was filtered through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give product C366 (6.9 mg).

### EXAMPLE 1-67

### Preparation of Compound C368

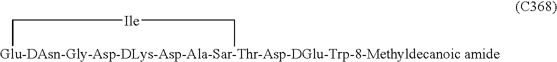

### Reaction 1

### Preparation of Resin-Glu(αOAllyl)-DAsn(NHTrt)-Gly-Asp(OtBu)-DLys(NHBoc)-Asp(OtBu)-Ala-Sar-Thr(OIleNHFmoc)-Asp(OtBu)-DGlu(OtBu)-Trp-8-Methyldecanoic amide (230)

Hydroxy-benzotriazole (20 mg), benzotriazole-1-yl-oxy-tris-(dimethylamino)-phosphoniumhexafluorophosphonate (BOP, 66 mg), and diisopropylethylamine (26 μL), were added to a solution of compound 131 (296 mg) in dimethylformamide (2 mL). Compound 56 (416 mg) was added and the mixture then shaken for 17 hours. A portion of the resin was removed and the coupling was judged to be incomplete using the Kaiser Test (vide supra). An additional portion of hydroxy-benzotriazole (20 mg), benzotriazole-1-yl-oxy-tris-(dimethylamino)-phosphoniumhexafluorophosphonate (BOP, 60 mg), and di-isopropylethylamine (30 μL), were added and the mixture was then shaken for 26 hours. Coupling was judged to be complete using the Kaiser Test so the resin was filtered through a glass sinter funnel, washed with dichloromethane (3×5 mL) and dried under reduced pressure, yielding compound 230.

Reaction 2: Preparation of

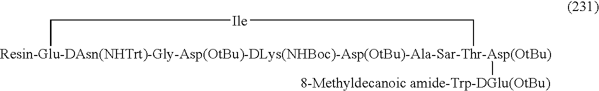

Compound 230 was placed under an argon atmosphere, and treated with a solution of tetrakis-(triphenylphosphine)palladium(0) (340 mg) in dichloromethane (9.25 mL), acetic acid (0.5 mL), and N-methylmorpholine (0.25 mL). The mixture was shaken for 4.5 hours at ambient temperature, filtered through a glass sinter funnel, and the solid was washed with 0.5% sodium thiocarbozoate in dimethylformamide (10 mL), 0.5% di-isopropylethylamine in dimethylformamide (10 mL), and dichloromethane (10 mL) then dried under reduced pressure. The resin was washed with DMF: piperidine 4:1 (10 mL) for 4 hours then filtered through a glass sinter funnel The solid was washed with dimethylformamide (10 mL) and dichloromethane (10 mL) then dried under reduced pressure The resin was suspended in N-methylmorpholine (3 mL), then 1-hydroxy-benzotriazole (135 mg) and 1,3-diisopropylcarbodiimide (157 μL) were added. The reaction was shaken for 17 hours, filtered, through a glass sinter funnel and washed well with N-methylmorpholine to give compound 231

Reaction 3: Preparation of compound (C368)

Dried compound 231 was suspended in dichloromethane (2.5 mL), trifluoroacetic acid (2.5 mL), ethanedithiol (125 μL), and triisopropylsilane (125 μL), and the reaction mixture was stirred for 4.5 hours at ambient temperature. The resin was filtered, through a glass sinter funnel washed with dichloromethane, and the combined filtrates were evaporated under reduced pressure. Crude product was then partitioned between diethyl ether (10 mL), and water (2.5 mL). The aqueous layer was freeze-dried to give crude product. The crude product was purified by reverse phase HPLC (C18 10 μM Jupiter column 250×21.2 mm) eluting with a gradient from 20% acetonitrile 0.5% formic acid: 80% water 0.5% formic acid to 80% acetonitrile 0.5% formic acid: 20% water 0.5% formic acid over 25 minutes. The product bearing fractions were combined and freeze-dried to give compound C368 (11.8 mg).

### EXAMPLE 1-68

### Stereoselective synthesis of 2S,3R-N-Fmoc-L-3-methyl-glutamic acid alpha allyl ester 232

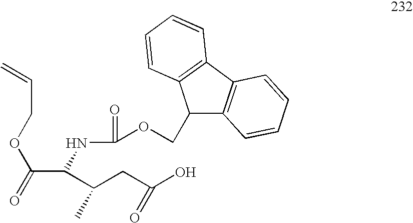

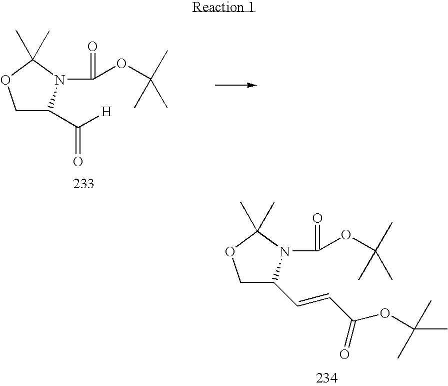

Tetra butyl ammonium iodide (39.4 g) was added to a solution of commercially available Gamer's aldehyde 233 (98 g) in 3M potassium carbonate (K₂CO₃, 100 mL) under a nitrogen atmosphere to give a heterogeneous solution. After 15 minutes tert-butyl-diethyl phosphonoacetate (130 g) was added and the reaction mixture was stirred vigorously for 18 hours. Water (500 ml) was added and the resultant mixture was extracted with methyl tert-butyl ether (MTBE, 3×250 mL). The combined organic fractions were combined, washed with saturated sodium chloride (1×250 mL), dried over magnesium sulfate (MgSO₄), filtered, and concentrated to give the crude product as a yellow oil. Purification by column chromatography on silica gel, eluting with ethyl acetate:hexane 1:9, gave the desired product 234 (95.3 g).

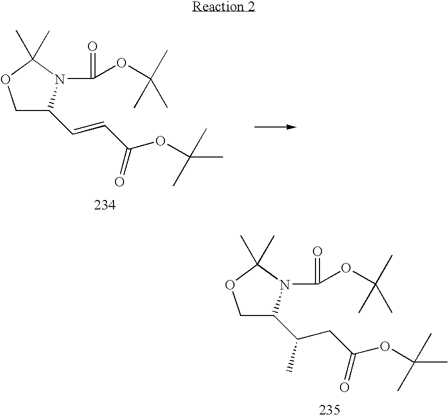

A solution of cuprous iodide (CuI, 137 g) in dry tetrahydrofuran (THF, 2250 mL) under a nitrogen atmosphere was cooled to −10° C. and stirred for 30 minutes. To this solution was added a 1.6 M solution of methyl lithium (MeLi) in diethyl ether (900 mL) such that the temperature remained below −10° C. The resultant mixture was stirred at −10° C. for 30 minutes then cooled to −78° C. and stirred for 45 minutes. Trimethyl silyl chloride TMSC1, (91 mL) was added such that the temperature remained below −78° C. then the reaction mixture was stirred for 15 minutes. A solution of the substrate ester 234 (85.45 g) in THF (250 ml) was added dropwise over one hour. The reaction mixture was stirred at −78° C. for one hour and allowed to warm to 40° C. before a quench solution of 90% saturated ammonium chloride (NH₄Cl): 10% ammonium hydroxide (NH₄OH, 1500 mL) was added slowly. The reaction mixture was stirred for 30 minutes and warmed to −30° C. before being worked up in 3 separate 1500 mL portions. Each portion was partitioned and the aqueous layer was extracted with MTBE (500 mL). The combined organic phases were filtered through celite and washed with the 90% saturated NH₄Cl:10% NH₄OH solution (4×400 mL), dried over sodium sulfate (Na₂SO₄), filtered, and concentrated to give the product. The volatiles were removed from the product under high vacuum to give the product 235 (85.45 g). Compound 235 was used without further purification.

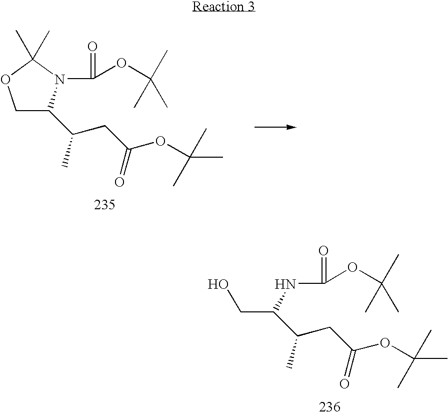

A solution of the oxazolidine 235 (70 g) in methanol (1800 mL) was cooled to 0° C. and stirred for one hour. Boron trifluoride acetic acid complex (BF₃₀.2HOAc, 450 mL) was added dropwise over two hours such that the internal temperature remained below 3° C. The reaction mixture was then quenched by the addition of 20% sodium bicarbonate (Na₂CO₃, 3000 mL) over two hours and the resultant solution was worked up in 5 separate 1000 mL portions. Each 1000 mL portion was extracted with dichloromethane (3×300 mL), the organic extracts were combined, washed with NaHCO₃, (1×300 mL) saturated sodium chloride (1×300 mL), dried over MgSO₄, filtered, and concentrated to give the crude product. The combined products were purified by column chromatography on silica gel, eluting with a gradient elution from 20% ethyl acetate:80% hexane to 50% ethyl acetate:50% hexane. Combining and evaporating the product bearing fractions gave the desired product 236 (36.3 g).

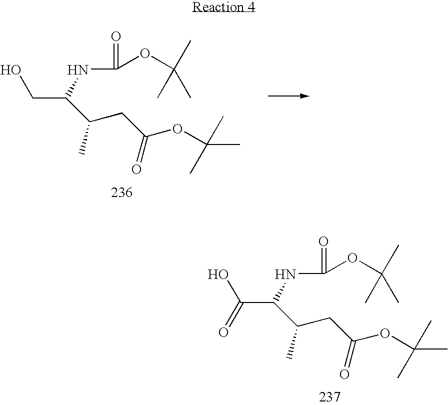

A solution of the alcohol 236 (24 g) in acetonitrile (238 mL) and water (29.7 mL) was cooled to 0° C., and periodic acid (52.2 g) was added in portions to maintain a temperature of 0° C. The reaction mixture was stirred at 0° C. for 45 minutes and chromium trioxide (CrO₃, 460 mg) was added. The reaction mixture was stirred for 15 minutes before being quenched by the slow addition of a 0.4 M dibasic sodium phosphate solution (Na₂HPO₄, 560 mL, pH 9.0). The resultant mixture was extracted with MBTE (4×300 mL), and the combined organic extracts were washed with saturated sodium chloride (1×250 mL), NaHCO₃ (1×250 mL), and saturated sodium chloride (1×250 mL). The organic portion was then dried over MgSO₄, filtered, and concentrated to give the crude product. Purification by preparative thin layer chromatography on silica gel, eluting with 20% ethyl acetate:80% hexane and extraction from silica gel with dichloromethane, gave the desired product 237 (15.32 g).

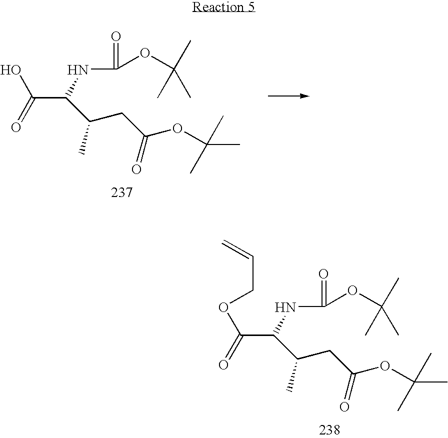

To a solution of the acid 237 (15.32 g) in N,N-dimethylformamide (DMF, 200 mL) was added potassium bicarbonate (KHCO₃, 9.66 g) and the resultant suspension was stirred for 15 minutes. A solution of allyl bromide (21 mL) in DMF (200 mL) was then added dropwise over 30 minutes and the reaction mixture was stirred for 19 hours. Water (500 mL) was added and the resultant mixture was extracted with ethyl acetate (5×200 mL), and the combined organic extracts were washed with water (2×200 mL), and saturated sodium chloride (1×200 mL). The organic portion was then dried over Na₂SO₄, filtered, and concentrated to give the crude product as a yellow oil. Purification by column chromatography on silica gel, eluting with ethyl acetate:hexane 1:4 gave the desired product 238 (9.2 g).

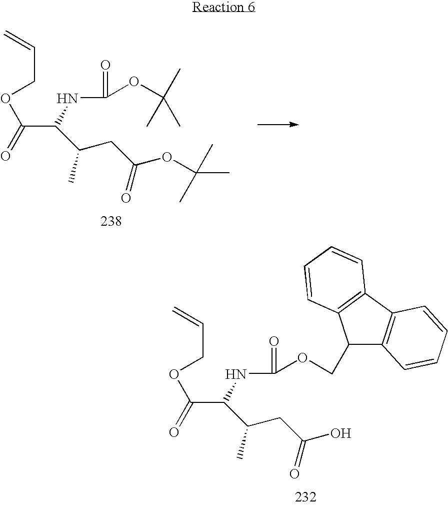

Trifluoroacetic acid (TFA, 25 mL) and triisopropyl silane (TIPS, 1 mL) was added to a solution of the ester 238 (9.2 g) in dichlromethane and the reaction mixture was stirred for 1 hour. The mixture was then concentrated under vacuum and the resultant residue was dissolved in hexane (100 mL) and re-evaporated three times. The residue was then dissolved in saturated NaHCO₃ (53 mL) and 1,4-dioxane (50 mL) and a solution of 9-Fluorenylmethoxycarbonyl-N-hydroxysuccinimide (FmocOSu, 9.52 g) in 1,4-dioxane (50 mL) was added dropwise over 30 minutes. During this time the reaction mixture became cloudy so a further portion of 1,4-dioxane (20 mL) added to give a heterogeneous solution that was stirred for a further 17 hours. The reaction mixture was filtered, and the residue was washed with 1,4-dioxane (50 mL). The combined organic fractions were evaporated and re-dissolved in ethyl acetate (250 mL) and acidified prior to washing with potassium sulfate (KHSO₄, 3×50 mL), and saturated sodium chloride (1×50 mL). The organic portion was then dried over Na₂SO₄, filtered, and concentrated to give the crude product. The product was purified by column chromatography on silica gel, using a gradient elution from 20% ethyl acetate:80% hexane to 40% ethyl acetate:60% hexane. Combining and evaporating the product bearing fractions gave the desired product 232 (6.32 g).

### EXAMPLE 1-69

### Stereoselective synthesis of 2S,3S-N-Fmoc-L-3-methyl-glutamic acid alpha allyl ester 239

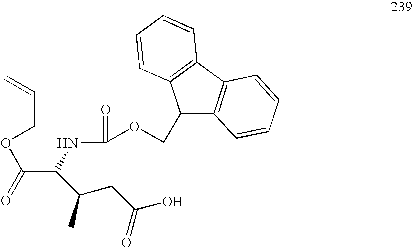

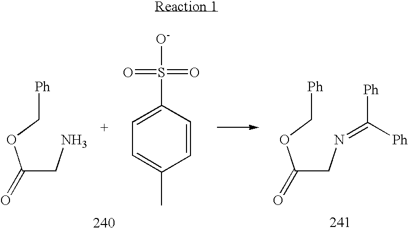

Glycine benzyl ester tosylate salt (6.75 g) was partitioned between dichloromethane (100 mL) and aqueous 10% w/v K₂CO₃ (100 mL). The aqueous portion was extracted with dichloromethane (2×50 mL), and the combined organic fractions were dried over MgSO₄, filtered and evaporated to a glassy solid (3.29 g). This solid was dissolved in dry dichloromethane (80 mL) and a solution of benzophenone imine (3.62 g) in dichloromethane (20 mL) was added. The resultant mixture was stirred at ambient temperature for 17 hours. The mixture was concentrated to an oil under vacuum, re-dissolved in ether (80 mL), and washed with water (2×40 mL). The organic layer was dried over MgSO₄, filtered and evaporated to give the crude product as a clear oil. Purification by recrystallization from warm ether/hexane gave pure 241 (3.82 g).

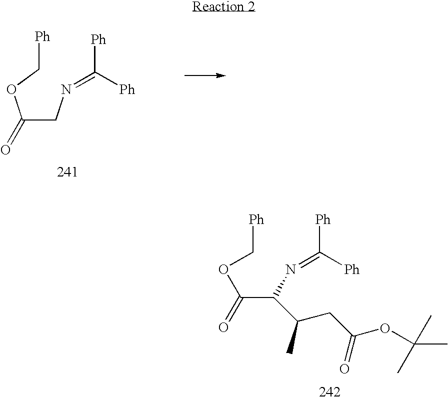

To a suspension of benzyl-N-(diphenylmethylene) glycinate 241 (5.7 g) and O-allyl-N-(9-anthracenylmethyl)cinchonidinium bromide (1.05 g) in dichloromethane (80 mL) cooled to −78° C. under a nitrogen atmosphere, was added cesium hydroxide (14.53 g). The mixture was stirred for 20 minutes and tert butyl crotonate (9.13 mL) was added dropwise so that the temperature remained at −78° C. After stirring at −78° C. for 2 hours the mixture was warmed to −50° C. for 30 minutes then the mixture was allowed to warm to ambient temperature over 2 hours. The mixture was then poured into diethyl ether (600 mL) and water (200 mL), partitioned, and the organic layer was washed with water (2×170 mL) and saturated sodium chloride (1×150 mL). The ether fraction was then dried over MgSO₄, filtered and evaporated to give the product 242 (4.46 g), which was used subsequently without further purification.

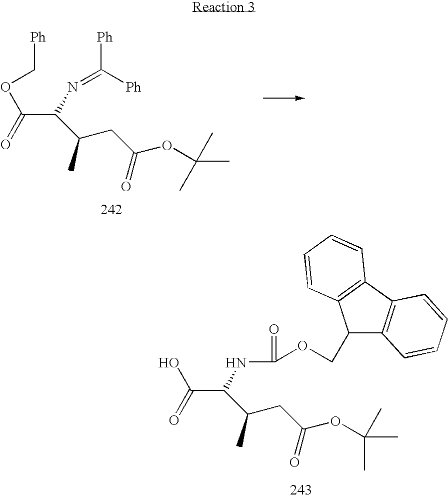

To a solution of the protected 3-methyl glutamate 64 (4.46 g) in tetrahydrofuran (250 mL) was added a solution of 10% w/v citric acid (120 mL) and the mixture was stirred for 17 hours. The solution was then concentrated under vacuum to remove the tetrahydrofuran and diethyl ether (100 mL) and 1N HCl (250 mL) were added. After partitioning, the aqueous layer was washed with diethyl ether (2×100 mL), basified to pH 14 by the addition of solid K₂CO₃, and extracted with ethyl acetate (4×100 mL). Acetic acid (3 mL), and 10% palladium on carbon (500 mg) were added to the combined ethyl acetate fractions and the resultant suspension was stirred under a hydrogen atmosphere for 16 hours. Methanol (300 mL) was added, and the reaction mixture was filtered through celite. The filtrate was evaporated to an oil, which was dissolved and evaporated first in ethyl acetate (300 mL) and then diethyl ether (300 mL) to give a white gel. This residue was dissolved in tetrahydrofuran (200 mL) and 10% w/v K₂CO₃ (100 mL), and 9-fluorenylmethoxycarbonyl-N-hydroxysuccinimide (5.83 g) was added. The reaction mixture was stirred for 22 hours, and concentrated under vacuum to remove the tetrahydrofuran. To the concentrated solution, diethyl ether (170 mL) and water (300 mL) were added. After partitioning, the aqueous layer was washed with diethyl ether (3×130 mL), acidified to pH 2 with concentrated HCl, and extracted with ethyl acetate (3×200 mL). The ethyl acetate fractions were then dried over MgSO₄, filtered and evaporated to give the product 243 (3.31 g), which was used subsequently without further purification.

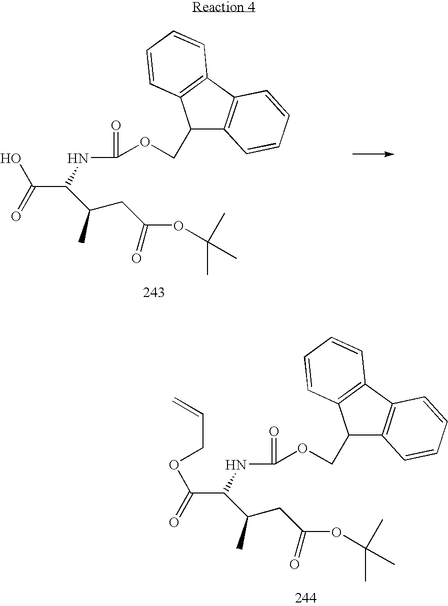

To a solution of 2S,3S-N-Fmoc-L-3-methyl-glutamic acid γ tert-butyl ester 243 (3.3 g) in dichloromethane (150 mL) was added N,N′diisopropylcarbodiimide polystyrene resin (10.8 g) and 4-dimethylaminopyridine (92 mg), and the reaction mixture was stirred for 5 minutes. Allyl alcohol (0.612 mL) was added, and the reaction mixture was stirred for a further 90 minutes. Filtration and evaporation of the solvent gave the desired diester 244 (2.02 g).

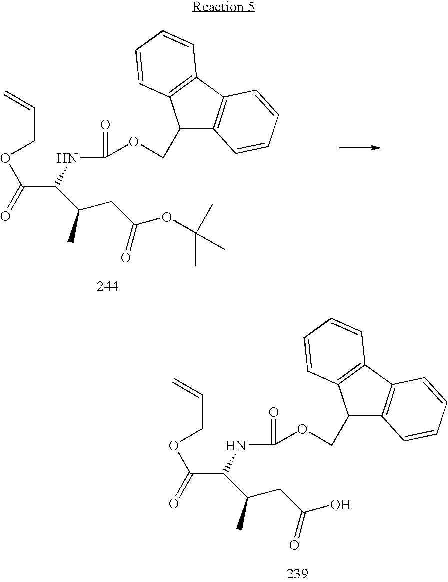

To a solution of the diester 244 in dichloromethane (42 mL), cooled to 0° C., was added triisopropylsilane (0.82 mL) and trifluoroacetic acid (4 mL). The reaction mixture was stirred at 0° C. for 10 minutes, warmed to ambient temperature, and stirred for 90 minutes. Hexane (600 mL) was added and the mixture was evaporated, the residue was dissolved in diethyl ether (150 mL) and 5% w/v K₂CO₃ (200 mL). The aqueous layer was washed with diethyl ether (2×80 mL), acidified to pH 2 with concentrated HCl, and extracted with ethyl acetate (3×100 mL). The ethyl acetate fractions were then dried over MgSO₄, filtered and evaporated to give the product 239 (1.48 g).

### EXAMPLE 1-70

### Preparation of 2S-N-Fmoc-L-3-O-(tert-butyldimethysilyl)-asparagine

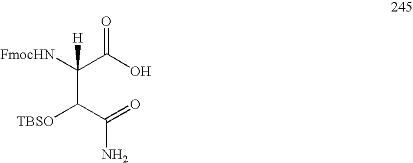

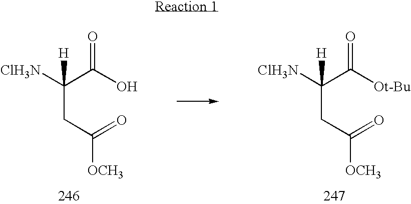

To a suspension of the commercially available aspartic acid ester 246 (2.75 g) in 70% perchloric acid (HClO₄, 3 mL) was added t-butyl acetic acid ester (100 mL). After 24 h, the solution was poured into saturated K₂CO₃ (200 mL). The resulting biphasic mixture was extracted with diethyl ether (3×100 mL) and the combined organic extract was washed with saturated K₂CO₃ (3×50 mL), dried over Na₂SO₄, filtered and concentrated under diminished pressure to give a clear colorless oil. The oil was then dissolved in cold (0° C.) diethyl ether (50 mL) and 1 N HCl in diethyl ether (15 mL) was added. After 20 min the solution was concentrated under diminished pressure to give 247 (2.0 g).

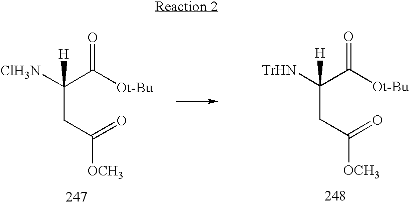

To a suspension of the diester 247 (4.78 g) in methyl tert-butyl ether (100 mL) was added saturated aqueous K₂CO₃ (100 mL). The resulting aqueous layer was washed with methyl tert-butyl ether (3×100 mL). The organic extract was combined and washed with saturated K₂CO₃ (2×100 mL), dried over Na₂SO₄, filtered and concentrated under diminished pressure. The resulting colorless oil was mixed with trityl chloride (5.57 g), CH₃CN (100 mL), and triethylamine (5.60 mL). After 16 h, the mixture was filtered and diluted with methyl tert-butyl ether (200 mL). The resulting organic extract was washed with 1N citric acid (3×100 mL), saturated NaHCO₃ (3×100 mL), saturated NaCl (3×100 mL), dried over Na₂SO₄, filtered, and concentrated under diminished pressure. Chromatography on base washed flash silica gel (20×4 cm) using 1:11 ethyl acetate-hexanes with 1% triethylamine present gave product 248 (7.12 g).

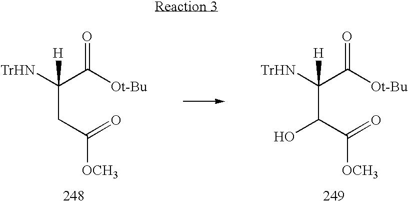

To a cooled (−78° C.) solution of 248 (2.22 g) in tetrahydrofuran (20 mL) was added 16.5 mL of 0.91 M potassium hexamethyldisilazane solution in tetrahydrofuran. After 1 h at −78° C., oxodiperoxy(pyridine)(1,3-dimethyl-3,4,5,6-tetrahydro-2(1H)-pyrimidinone)molybdenum (IV) (MOOPD, 3.68 g, obtained from STREM Chemicals, Inc; see Anderson, J C. and Smith, S C., 1990, Synlett 107-109 for the synthesis of this reagent) was added in portions. The mixture was stirred for 1 h at −78° C., allowed to warm up to −55° C., and stirred for an additional hour. The mixture was quenched with saturated aqueous Na₂SO₃ (20 mL) and warmed to room temperature. The resulting biphasic mixture was washed with methyl tert-butyl ether (3×100 mL) and the organic layers were combined and washed with 1 N citric acid (3×50 mL), saturated aqueous NaHCO₃ (3×50), and saturated aqueous NaCl (3×50), dried over Na₂SO₄, filtered, and concentrated under diminished pressure. Chromatography on flash silica gel (20×3 cm) using 1:5 ethyl acetate-hexanes gave product 249 (1.64 g).

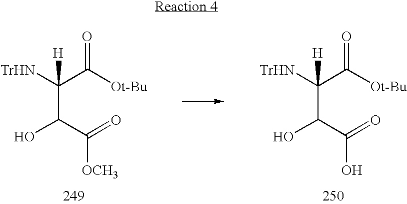

To a solution of 249 (650 mg) in 1:1 dioxane-water (50 mL) was added lithium hydroxide (507 mg). After 1 h the solution was washed with methyl tert-butyl ether (3×50 mL) and the resulting aqueous layer was acidified to pH ˜4 with 1N citric acid. The resulting solution was extracted with methyl tert-butyl ether (3×100 mL). The organic phase was washed with 1 N citric acid (3×100 mL), and saturated NaCl (3×100 mL), dried over Na₂SO₄, filtered, and concentrated under diminished pressure to give 250 (630 mg).

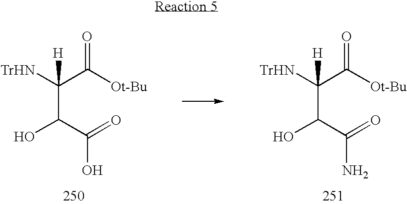

To a solution of 250 (650 mg), benzotriazol-1-yloxy-tris(dimethylamino)-phosphonium hexafluorophosphate (997 mg), and NH₄Cl (153 mg) in dimethylformamide (30 mL) was added diisopropylethylamine (0.78 mL). After 1 h, ethyl acetate (150 mL) was added and the resulting solution was washed with 10% K₂CO₃ (3×100 mL), water (3×100 mL), 1 N citric acid (3×100 mL), and saturated NaCl, dried over Na₂SO₄, filtered, and concentrated under diminished pressure to yield 251 (640 mg).

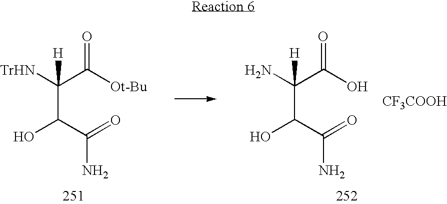

To a solution of 251 (1.91 g) in dichloromethane (5 mL) was added water (1 mL) followed by trifluoroacetic acid (10 mL). After 4 h, the solution was concentrated under diminished pressure and the remaining slurry was concentrated twice from toluene. The resulting solid was triturated with diethyl ether and the resulting solid was filtered and washed with diethyl ether. The resulting solid was dried under diminished pressure to give 252 (952 mg).

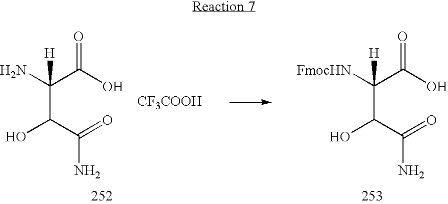

To a solution of a solution of 9-Fluorenylmethoxycarbonyl-N-hydroxysuccinimide (3.37 g) in 1-4-dioxane (50 mL). After 16 h, the resulting solution was diluted with aqueous 5% K₂CO₃ solution (25 mL) and extracted with diethyl ether (3×50 mL). The resulting aqueous extract was acidified to pH 2 with a 1 N HCl solution and diethyl ether (50 mL) was added. The resulting solid was partitioned between the acidic solution and diethyl ether. The solid was collected and washed with 1N HCl and diethyl ether to yield 253 (1.50 g).

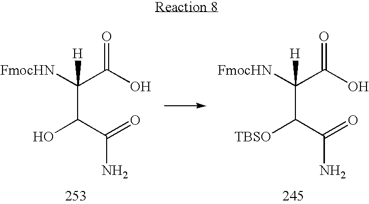

To a solution of 253 (370 mg) in dimethylformamide (10 mL) was added tert-butyldimethylsilyl chloride (300 mg), followed by imidazole (200 mg). After 8 h, the solution was diluted with ethyl acetate and washed with 1 N HCl (3×100 mL) and saturated sodium chloride, dried over Na₂SO₄, filtered, and concentrated under diminished pressure. Chromatography on flash silica gel (25×2 cm) using 19:1:0.1 CH₂Cl₂:MeOH:AcOH as eluent gave 245 (300 mg).

### EXAMPLE 1-71

### Preparation of 2S-N-Fmoc-L-(3-methoxy)-β-3-tert-butyl aspartic acid ester 254

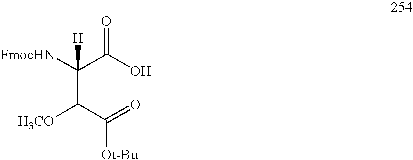

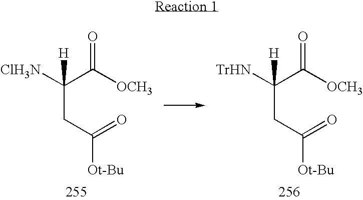

To a suspension of commercially available diester 255 (4.78 g) in methyl tert-butyl ether (100 mL) was added saturated aqueous K₂CO₃ (100 mL). The resulting aqueous layer was washed with methyl tert-butyl ether (3×100 mL). The organic extract was combined and washed with saturated K₂CO₃ (2×100 mL), dried over Na₂SO₄, filtered and concentrated under diminished pressure. The resulting colorless oil was dissolved in a solution of trityl chloride (5.57 g), and triethylamine (5.60 mL) in acetonitrile (100 mL). After 16 h, the mixture was filtered and diluted with methyl tert-butyl ether (200 mL). The resulting organic extract was washed with 1N citric acid (3×100 mL), saturated NaHCO₃ (3×100 mL), and saturated NaCl (3×100 mL), dried over Na₂SO₄, filtered, and concentrated under diminished pressure. Chromatography on base washed flash silica gel (20×4 cm) using 1:11 ethyl acetate-hexanes with 1% triethylamine present gave 256 (7.12 g).

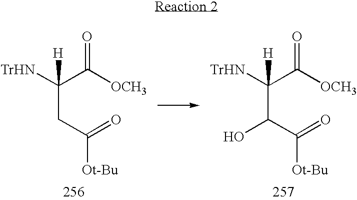

To a cooled (−78° C.) solution of 256 (2.22 g) in tetrahydrofuran (20 mL) was added 16.5 mL of 0.91 M potassium hexamethyldisilazane solution in tetrahydrofuran. After 1 h at −78° C., oxodiperoxy(pyridine)(1,3-dimethyl-3,4,5,6-tetrahydro-2(1H)-pyrimidinone) molybdenum (IV) (MoOPD, obtained from STREM Chemicals, Inc; 3.68 g, see Anderson, J C. and Smith, S C., 1990, Synlett 107-109 for the synthesis of this reagent) was added in portions. The mixture was stirred for 1 h at −78° C., allowed to warm up to −55° C. and stirred for an additional hour. The mixture was quenched with saturated aqueous Na₂SO₃ (20 mL) and warmed to room temperature. The resulting biphasic mixture was washed methyl tert-butyl ether (3×100 mL). The organic layers were combined and washed with 1 N citric acid (3×50 mL), saturated aqueous NaHCO₃ (3×50), and saturated aqueous NaCl (3×50), dried over Na₂SO₄, filtered, and concentrated under diminished pressure. Chromatography on flash silica gel (20×3 cm) using 1:5 ethyl acetate-hexanes gave 257 (1.64 g).

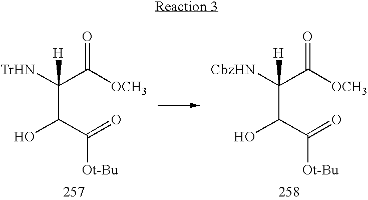

To a solution of 257 (4.61 g) in dichloromethane (100 mL) was added trifluoroacetic acid (2 mL). After 1 h, the resulting solution was concentrated under diminished pressure, and aqueous 5% K₂CO₃ (50 mL) was added followed by a solution of benzyloxycarbonyl-N-hydroxysuccinimide (2.49 g) in dioxane (50 mL). After 16 h, the resulting solution was extracted with ethyl acetate (3×100 mL). The organic extract was washed with 1 N citric acid (3×50 mL), saturated NaHCO₃ (3×50 mL) and NaCl (3×50 mL), dried over Na₂SO₄, filtered and concentrated under diminished pressure. Chromatography on flash silica gel (25×3 cm) using 1:3 ethyl acetate-hexanes gave 258 (2.01 g).

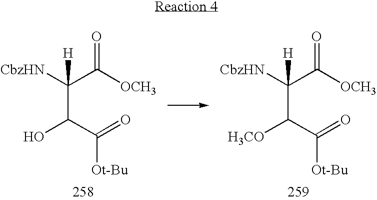

To a cold (0° C.) suspension of 258 (353 mg) and silver oxide (462 mg) in tetrahydrofuran (25 mL) was added iodomethane (0.62 mL). The mixture was allowed to warm up to room temperature over 4 h. After 48 h, the suspension was filtered through Celite and concentrated under diminished pressure. Chromatography on flash silica gel (25×2 cm) using 1:3 ethyl acetate-hexanes gave 259 (300 mg).

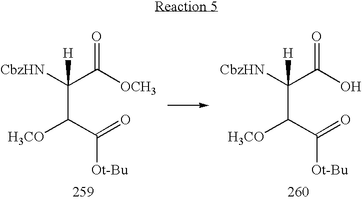

To a cold (0° C.) solution containing 259 (300 mg, 0.81 mmol) in 25 mL of dioxane was added 240 mg (10 mmol) of LiOH in 25 mL of water. After 1 h, the mixture was acidified to pH 4 with 1 N citric acid, and extracted with ether (3×50 mL). The resulting organic extract was washed with 1 N citric acid (3×50 mL), and saturated NaCl (3×30 mL) dried over Na₂SO₄, filtered and concentrated under diminished pressure to give 260 as a clear, colorless oil (280 mg).

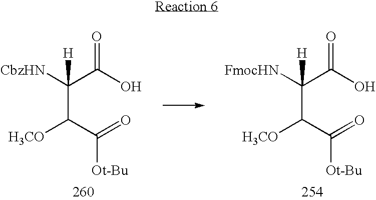

Compound 260 is converted to 254 by treatment of an ethyl acetate solution of 260 with 10% palladium on carbon, under a hydrogen atmosphere as previously described for the conversion of 242 to 243 followed by an amine protection as previously described for conversion of 252 to 253.

### EXAMPLE 1-72

### Preparation of Nα-(Allyloxycarbonyl)-isoleucine 124

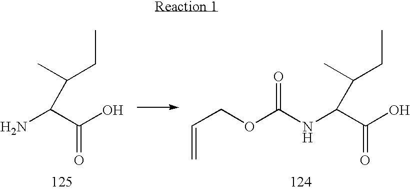

Commercially available Isoleucine (22 g) was added to a solution of allyloxycarbonyl oxysuccinimide (AllocOSu, 51 g) in tetrahydrofuran (150 mL). Ten percent K₂CO₃ aqueous solution (100 mL) was added to this suspension and the mixture was stirred for 17 hours before concentrating to approximately 120 ml under reduced pressure. The solution was added to 10% K₂CO₃ aqueous solution (100 mL) and water (200 ml) and washed with diethyl ether (4×150 mL). The aqueous portion was then acidified to pH 1 and extracted with dichloromethane (4×200 mL). Combined acidic dichloromethane washes were dried with anhydrous MgSO₄ and evaporated to crude product (38.1 g). Purification by column chromatography on silica gel, (eluting with dichloromethane/methanol gradient of 100% dichloromethane to dichloromethane: methanol 9:1) followed by evaporation of the solvent, gave the compound 124 as a yellow oil (36 g).

### EXAMPLE 2-1

### Construction of an S. Roseosporus-Based in-Trans Expression System for the Production of the Novel Biosynthetic Pathways

For the expression of the hybrid non-ribosomal polypeptide synthetase (NRPS) pathways, a version of the S. roseosporus high daptomycin-producing strain (NRRL 11379) that lacked all of the NRPS genes was constructed. The hybrid pathways were conjugated into this strain on BAC-based vectors which integrated site-specifically in a neutral site of the S. roseosporus genome at a φC31 attB site.

To delete all the proposed NRPS genes from S. roseosporus a deletion cassette was constructed that contained flanking DNA from upstream of dptEF and downstream of dptH (FIG. 2).

Flanking regions from upstream of dptEF (5′) and downstream of dptH (3′) were cloned around a selection cassette containing tsr (thiostrepton resistance) and cat (chloramphenicol resistance). The 5′ fragment was 1478 bp long and the 3′ fragment was 1862 bp long. These two fragments were cloned into a copy of pUC19 (New England Biolabs) that already contained the tsr and cat resistance cassettes to create the deletion cassette. This cassette was then transferred to a delivery plasmid called pRHB538 (Hosted, T. J. and Baltz, R. H., 1997, J. Bacteriol. 179(1): 180-6), which contains a temperature sensitive origin of replication and a dominant allele of rpsL (streptomycin sensitive). This plasmid was introduced into a S. roseosporus strain carrying a recessive rpsL allele that confers streptomycin resistance. This recombinant strain was then incubated overnight in a broth culture before the cells were spread on plates containing streptomycin plus thiostrepton and incubated at 39° C. Under these conditions only those strains that have exchanged the deletion cassette (containing tsr and cat) for the dptA-H locus via homologous recombination survived the selection; all other genotypes were eliminated.

PCR and Southern blots confirmed the genotype of the dptA-H deletion mutants. The PCR fragments were designed to be amplified from primers that lay outside the 5′ and 3′ flanking regions and inside the tsr and cat genes. In this way, the PCR products can only be formed when the cell has exchanged the deletion cassette for the dptA-H locus. The Southern blots provided further confirmation that dptA-H had been deleted and that no aberrant integrations or recombination had occurred around this locus. Once the dptA-H deletions were confirmed genetically they were then tested to see if they were true null mutants phenotypically. This strain was then designated S. roseosporus UA43 1.

### EXAMPLE 2-2

### Fermenting Streptomyces roseosporus

Spores of the Streptomyces roseosporus UA431 were harvested by suspending a 10 day old slant culture of medium A (2% irradiated oats (Quaker), 0.7% tryptone (Difco), 0.2% soya peptone (Sigma), 0.5% sodium chloride (BDH), 0.1% trace salts solution, 1.8% agar no. 2 (Lab M), 0.01% apramycin (Sigma)) in 5 mL 10% aqueous glycerol (BDH)). One mL of this suspension, in a 1.5 mL cryovial, comprises the starting material, which was retrieved from storage at −135° C. A pre-culture was produced by aseptically placing 0.3 mL of the starting material onto a slant of medium A and incubating for 9 days at 28° C.

A seed culture was generated by aseptically treating the pre-culture with 4 mL of a 0.1% Tween 80 (Sigma) solution and gently macerating the slope surface to generate a suspension of vegetative mycelium and spores. A two mL aliquot of this suspension was transferred into a 250 mL baffled flask containing 40 mL of nutrient solution S (1% D-glucose (BDH), 1.5% glycerol (BDH), 1.5% soya peptone (Sigma), 0.3% sodium chloride (BDH), 0.5% malt extract (Oxoid), 0.5% yeast extract (Lab M), 0.1% Junlon PW100 (Honeywell and Stein Ltd), 0.1% Tween 80 (Sigma), 4.6% MOPS (Sigma) adjusted to pH 7.0 and autoclaved)) and shaken at 240 rpm for 44 hours at 30° C.

Production cultures were generated by aseptically transferring 5% of the seed culture to baffled 250 mL flasks containing 50 mL medium P (1% glucose (BDH), 2% soluble starch (Sigma), 0.5% yeast extract (Difco), 0.5% casein (Sigma), 4.6% MOPS (Sigma) adjusted to pH 7 and autoclaved)) and shaken at 240 rpm for up to 7 days at 30° C.

### EXAMPLE 2-3

### Analysis of the A219 78C Lipopeptides from Fermentations of the Streptoimyces Roseosporus

Production cultures described in Example 2-2 were sampled for analysis by aseptically removing 2 mL of the whole culture and centrifuging for 10 minutes prior to analysis. Volumes up to 50 microlitres of the supernatant were analyzed to monitor for production of the native lipopeptides (A21978C) as produced by Streptomyces roseosporus. This analysis was performed at ambient temperature using a Waters Alliance 2690 HPLC system and a 996 PDA detector with a 4.6×50 mm Symmetry C8 3.5 μm column and a Phenomenex Security Guard C8 cartridge. The gradient initially holds at 90% water and 10% acetonitrile for 2.5 minutes, followed by a linear gradient over 6 minutes to 100% acetonitrile. The flow rate is 1.5 mL per minute and the gradient is buffered with 0.01% trifluoroacetic acid. By day 2 of the fermentation, production of three of the native lipopeptides, A21978C₁, A21978C₂ and A21978C₃, with UV/visible spectra identical to that of daptomycin, was evident, as shown by HPLC peaks with retention times of 5.62, 5.77 and 5.90 minutes (?max 223.8, 261.5 and 364.5 nm) under the analytical conditions stated. The lipopeptides then remained evident in the fermentation at each sample point during the 7-day period. Total yields of lipopeptides A21978C₁, A21978C₂ and A21978C₃ ranged from 10-20 mg per liter of fermentation material.

Liquid chromatography-mass spectrometry (LC-MS) analysis was performed on a Finnigan SSQ710c LC-MS system using electrospray ionization in positive ion mode, with a scan range of 200-2000 daltons and 2 second scans. Chromatographic separation was achieved on a Waters Symmetry C8 column (2.1×50 mm, 3.5 μm particle size) eluted with a linear water-acetonitrile gradient containing 0.01% formic acid, increasing from 10% to 100% acetonitrile over a period of six minutes after a initial delay of 0.5 minutes, then remaining at 100% acetonitrile for a further 3.5 minutes before re-equilibration. The flow rate was 0.35 mL/minute and the method was run at ambient temperature.

The identification of the three native lipopeptides was confirmed in the controls (S. roseosporus wild type), as indicated by molecular ions ([M+H]⁺) at m/z of 1634.7, 1648.7 and 1662.7, which is in agreement with the masses reported for the major A21978C lipopeptide factors A21978C₁, A21978C₂ and A21978C₃, respectively, produced by Streptomyces roseosporus (Debono et al., 1987, J. Antibiotics 40: 761-777). The UA431 mutants failed to produce any of the A21978C lipopeptides confirming that they were true null mutants.

### EXAMPLE 2-4

### Constructing pDA300 and Complementing the S. Roseosporus DptA-H Deletions

Unlike yeast and some naturally competent bacteria, linear DNA fragments do not readily transform Escherichia coli. This is in part due to the degradation of foreign DNA by intracellular exonucleases such as RecBCD (Lorenz, M. G., and Wackemagel, W., 1994, Microbiol. Rev. 58: 563). Traditionally, homologous recombination was either achieved by using mutant strains lacking RecBCD (Jasin, M., and Schimmel, P., 1984, J. Bacteriol 159: 783) or by delivering DNA with the help plasmid vectors that cannot replicate in the host under restrictive conditions (Link, A. J. et al., 1997, J. Bacteriol. 179: 6228). Recombination events remain rare and require kilobases of homology.

Recently, several laboratories have developed strains that take advantage of the bacteriophage λ-induced “hyper-recombination” state (Datsenko, K. A., and Wanner, B. L., 2000, Proc. Nat. Acad Sci U.S.A. 97: 6640; U.S. Pat. Nos. 6,355,412 and 6,509,156B; Yu, D., et al., 2000, Proc. Nat. Acad Sci U.S.A. 97: 5978). Recombination between DNA molecules with as little as 40-50 bp of identical sequence takes place even when using linear DNA. The λ Red genes (exo, bet and gam) cause the enhancement of the recombination rate. The λ-exonuclease and the β-protein are responsible for recombination through repair of double-strand breaks, whereas the gam gene product binds to the host RecBCD complex and inhibits its functions (Murphy, K., 1998, J. Bacteriol. 180: 2063). We refer to this technique as the “Red” system or Red-mediated recombination system.

Using the “Red” system a pDA300 (a truncated version of B12:03 A05 that contains only the dptA-H genes) was constructed. This plasmid was constructed from B 12:03 A05 (a BAC plasmid that contains all of the dpt biosynthetic gene cluster, which was isolated from a chromosomal library of S. roseosporus (Miao et al, 2005, Microbiology 151: 1507-1523), all of the genes upstream of dptA-H and all of genes downstream of dptA-H were deleted using homologous recombination via the Red-mediated recombination system. This was achieved by introducing B12:03 A05 into an E. coli strain carrying the Red genes on a plasmid (pKD78, Datsenko, K A., and Wanner, B L., 2000, Proc. Nat. Acad Sci U.S.A. 97: 6640). This strain was then transformed by PCR products for the tet resistance gene that were flanked by oligonucleotides with homology to either the upstream or downstream regions of the dpt cluster. Once constructed, pDA300 was introduced into UA431 by conjugation to create strain UA493. Plasmid pDA300 contains oriT from plasmid RK2 (Baltz, 1998, Trends in Microbiol. 6: 76-83 (1998), incorporated herein by reference in its entirety) for conjugation from E. coli to S. roseosporus. Plasmid pDA300 is introduced into S. roseosporus by conjugation from E. coli S17.1, or a strain containing a self-replicating plasmid RK2 (Id.). S. roseosporus UA493 was fermented and analyzed using the techniques described in Examples 2-2 and 2-3 respectively.

The identification of the three native lipopeptides was confirmed, as indicated by molecular ions ([M+H]⁺) at m/z of 1634.7, 1648.7 and 1662.7, which is in agreement with the masses reported for the major A21978C lipopeptide factors A21978C₁, A21978C₂ and A21978C₃, respectively, produced by Streptomyces roseosporus (Debono et al., 1987, J. Antibiotics 40: 761-777). This demonstrated that the pDA300 was able to successfully complement the dptA-H deletion to restore lipopeptide production in UA493.

### EXAMPLE 2-5

### Exchange of a Non-Ribosomalpeptide Synthetase (NRPS) Subunit for One that Catalyzes the Incorporation of different Amino Acid(s)

The gene that encodes the third subunit of the daptomycin NRPS (see FIG. 1) contains two modules that encode the specificity for incorporation of amino acids 12 (3-methyl-glutamic acid (3-MeGlu)) and 13 (L-kynurenine (L-Kyn)). The gene that encodes the third subunit for the biosynthesis of the cyclic lipopeptide CDA (Kempter et al, 1997, Angew. Chem. Int. Ed. Engl. 36: 498-501; Chong et al., 1998, Microbiology 144: 193-199; each of which is incorporated by reference herein in its entirety) also encodes the last two amino acids, in this case amino acids 10 (3-MeGlu) and 11 (L-tryptophan (L-Trp); FIG. 1). A derivative of daptomycin containing L-Trp instead of L-Kyn in position 13 was generated by deleting gene dptD, and by replacing it with the gene that encodes PS3 for CDA (Hojati et al., 2002, Chem. Biol. 9(11):1175-87). The vector pMF23 expressed the PS3 gene from a strong promoter (e.g., the ernEp* promoter; Baltz, 1998, Trends Microbiol. 6: 76-83, incorporated herein by reference in its entirety), and when introduced in to S. roseosporus via interspecies conjugation (Baltz, 1998, Trends Microbiol. 6: 76-83) before site-specifically inserting into a neutral site in the S. roseosporus genome, allowed cdaPS3 to complement the dptD mutation and resulted in the production of the altered daptomycin with L-Trp replacing L-Kyn to give compound C1, compound C2, and compound C3. The recombinant strain was fermented and the product(s) of the recombinant strain were analyzed by LC-MS as described in Examples 2-2 and 2-3. Similar experiments were performed where the dptD deletion was complemented by the gene that encodes the third subunit for the biosynthesis of the cyclic lipopeptide A54145 (pMF30 is a derivative of pHM11a that contains lptD expressed from ermEp* (Motamedi et al., 1995, Gene 160: 25-31) which also encodes the last two amino acids, in this case amino acids 12 (3-MeGlu) and 13(L-isoleucine (L-11e) or L-valine (L-Val)). Two derivatives of daptomycin containing either L-Ile or L-Val instead of L-Kyn in position 13 were generated by disrupting gene dptD, and by replacing it with the gene that encodes lptD for A54145 (compounds C4, C5, C6, C7, C8, C9).

Similar manipulations are performed for trans-complementation for other subunits, i.e. to generate a disruption or deletion in a subunit of the daptomycin biosynthetic gene cluster or the A54145 biosynthetic gene cluster, and then complement in trans by one or more natural or modified subunits from an NRPS (the latter can include trans-complementation by modified versions of daptomycin or A54145 biosynthetic gene cluster subunits). Trans-complementation between the NRPS subunits then leads to production of a novel nonribosomal peptide which can be analyzed for as described in previous examples.

To perform a trans-complementation experiment using portions of the daptomycin or A54145 biosynthetic gene cluster and the calcium dependent antibiotic (CDA) biosynthetic gene cluster, the set of daptomycin biosynthetic genes, or the set of daptomycin biosynthetic genes and accessory genes, such as those contained on the BAC clone B12:03 A05, are introduced by transformation or conjugation into other natural or engineered strains or species of actinomycetes. The recipients may be known producers of secondary metabolites or uncharacterized strains, or may be generated by recombinant techniques to carry biosynthetic pathways other than that for biosynthesis of daptomycin. The transformants or ex-conjugants are fermented in a variety of media and whole broth or extracts thereof are screened for either novel daptomycin-like compounds or biological activity against daptomycin-resistant tester organisms.

The complementation is often facilitated by inactivation of some of the subunit genes in the daptomycin or A54145 biosynthetic pathway (as is described above for the deletion of dptD and complementation by either cdaPS3 or lptD). Sequences encoding a subunit of the NRPS are deleted or replaced by a marker gene to form a modified NRPS biosynthetic pathway; this can be achieved either in the original producing strain (S. roseosporus for daptomycin, S. fradiae or S. refuineus for A54145, S. coelicolor for CDA) or plasmids carrying these biosynthetic pathways.

To produce the novel lipopeptide, homologous recombination across flanking DNA sequences was used to exchange the bulk of the coding region of dptD in pDA300 for a heterologous marker gene. To perform the homologous recombination, two oligonucleotides were designed to amplify the regions directly upstream (“5“fragment”) and downstream (“3′ fragment”) of dptD. The 5′ and 3′ fragments were amplified from chromosomal DNA of S. roseosporus using the following primer sets with 5′-terminal extensions in which unique restriction sites have been introduced (underlined):

5′ fragment (1122 bp):

(SEQ ID NO: 1)

5′ GCG AAG CTT CTG GTG GCG CAT CAC CTG G 3′

(SEQ ID NO: 2)

5′ GCT CTA GAT GGA AGT ATG TCC TCC ATC GC 3′
3′ fragment (1535 bp):

(SEQ ID NO: 3)

5′ CGG ATC CCG CCG GCA CCT GAC CC 3′

(SEQ ID NO: 4)

5′ CCG AAT TCC GCC TCC GAG TAC ATC GAG G 3′

The amplified fragments were cloned in succession into the corresponding unique sites in the multiple cloning site of pNEB 193 (New England Biolabs). The resulting construct, pSD002, was confirmed by restriction digest analysis for orientation, and by sequencing for the absence of errors in the portions generated by PCR. A SpeI fragment containing the marker gene, ermE (erythromycin resistance gene; see Hopwood, supra) was inserted into pSD002 at an XbaI site and verified by restriction digest analysis. The resulting plasmid, pSD005, thus includes a cassette composed of ermE flanked by DNA stretches homologous to DNA sequences upstream and downstream of dptD. Once inserted into the daptomycin biosynthetic gene cluster pathway by homologous recombination, this cassette would essentially replace all of dptD, except for the first 31 bp and the last 12 bp, with ernzE. The region comprising the replacement cassette was then subcloned into a vector (a cloning site-modified version of pRHB538; (Hosted and Baltz, 1997, J. Bacteriol. 179: 180-186) carrying a temperature-sensitive replication origin and rpsL (a gene conferring sensitivity to streptomycin) to create pSDO30, the final plasmid in the series for introduction into S. roseosporus.

The plasmid, pSDO30, was introduced into S. roseosporus by interspecies conjugation (Baltz, 1998, Trends Microbiol., 6: 76-83). Each plate was then flooded with 1 mL of water containing 1.25 mg of erythromycin, resulting in a final concentration of 50 μg/ml once the liquid was absorbed into the media. Erythromycin-resistant colonies arising on the transformation plate after 7 days were inoculated into 25 mL of TSB (Hopwood, supra) plus erythromycin and incubated at 30° C. for 48 hours. The mycelium was harvested, and 1/10th of the mycelial mass was macerated and transferred to a new aliquot of 25 mL TSB plus erythromycin. The resultant solution was then incubated at 40° C. to select against the temperature-sensitive replicon of pSDO30. After 48 hours, the mycelium was harvested by centrifugation, macerated and resuspended in a final volume of 2 mL TSB. This suspension (100 μL) was spread on SPMR plates (Babcock et al., 1988, J. Bacteriol. 170: 2802-2808) containing 50 μg/mL erythromycin and 30 μg/mL of streptomycin. Colonies that survived were screened and shown to have the correct genotype by PCR to identify strains such as S. roseosporus UA378, in which ermE had successfully replaced dptD. This mutant was then complemented in-trans by initially dptD, where dptD was expressed from the expression plasmid pHM11a (Motamedi H, et al., 1995, Gene 160(1): 25-31) under the control of the constitutive promoter ermnEp*.

Starting material of UA378 was regenerated by suspending a 10 day old slope culture of medium A (see “Practical Streptomyces Genetics” by Kieser T., et al., John Innes Foundation, Norwich, 2000, herein “Kieser”; 2% irradiate oats (Quaker), 0.7% tryptone (Difco), 0.2% soya peptone (Sigma), 0.5% sodium chloride (BDH), 0.1% trace salts solution, 1.8% agar no. 2 (Lab M), 0.01% apramycin (Sigma) in 5 mL 10% aqueous glycerol (BDH)). A 1.5 mL cryovial containing 1 mL of starting material was retrieved from storage at −135° C. and thawed rapidly. A pre-culture was produced by aseptically placing 0.3 mL of the starting material onto a slope of medium A and incubating for 9 days at 28° C. Material for inoculation of the seed culture was generated by aseptically treating the preculture with 4 mL of a 0.1% Tween 80 (Sigma) solution and gently macerating the slope surface to generate a suspension of vegetative mycelium and spores.

A seed culture was produced by aseptically placing 1 mL of the inoculation material into a 2 L baffled Erlenmeyer flask containing 250 mL of nutrient solution S (see Kieser, supra) shaken at 240 rpm for 2 days at 30° C.

A production culture was generated by aseptically transferring the seed culture to a 20 L fermenter containing 14 liters of nutrient solution P (see Kieser, supra). The production fermenter was stirred at 350 rpm, aerated at 0.5 vvm, and temperature controlled at 30° C. After 20 hours incubation a 50% (w/v) glucose solution was fed to the culture at 5 g/hr throughout the fermentation.

After 40 hours incubation, a 50:50 (w/w) blend of decanoic acid:methyl oleate (Sigma and Acros Organics, respectively) was fed to the fermenter at 0.5 g/hr for the remainder of fermentation. The culture was harvested after 112 hours, and the biomass was removed from the culture supernatant by batch processing through a bowl centrifuge.

The biomass from the 20 L fermentation was discarded and the clarified liquor was applied to an open glass column, packed with Mitsubushi HP20 resin (60×300 mm) and conditioned with methanol and water. Prior to elution, the column was washed with 2 L of water followed by 2 L of methanol/water (1:4). The column was then eluted with 2 L of methanol/water (4:1) followed by 1 L methanol, and collected as two separate fractions.

Liquid chromatography-mass spectroscopy (LC-MS) electrospray ionization (ESI) analysis indicated that both fractions contained the A21978C/CDA hybrid molecules, and the less complex methanol/water (4:1) fraction was processed further. This was evaporated under vacuum to an aqueous residue and then made up to 500 mL with water. It was then back extracted with 3×500 mL of ethyl acetate in a 2 L separating funnel, to give an aqueous and organic fraction. LC-MS (ESI) indicated that the hybrid molecules were absent from the organic phase and it was discarded. The aqueous fraction was lyophilized overnight.

The hybrid molecules were purified by preparative high performance liquid chromatography (HPLC) using a Waters Prep LC system and a Waters 40×200 mm Nova-Pak C18 60 Å 6 μm radially-compressed double cartridge with 40×10 mm guard. The freeze-dried material was dissolved in water and purified using a gradient method. This method held at 90% water and 10% acetonitrile for 2 minutes and was followed by a linear gradient over 13 minutes to 25% water and 75% acetonitrile. The flow was 55 mL/min and the whole gradient was buffered with 0.04% trifluoroacetic acid. Fractions were collected and analyzed by LC-MS on a Finnigan SSQ710c LC-MS system using electrospray ionisation (ESI) in positive ion mode, with a scan range of 200-2000 daltons and 2 second scans. Chromatographic separation for this LC-MS analysis was achieved on a Waters Symmetry C8 column (4.6×50 mm, 3.5 μm particle size) eluted with a linear water-acetonitrile gradient containing 0.01% formic acid, increasing from 10% to 100% acetonitrile over a period of six minutes after an initial delay of 0.5 minutes, then remaining at 100% acetonitrile for a further 3.5 minutes before re-equilibration. The flow rate was 1.5 mL/minute and the method was run at ambient temperature.

The analysis identified compound C1 and compound C2. Both fractions required further purification prior to NMR studies. Compound C1 was further purified using an isocratic method with 60% water and 40% acetoniirile buffered with 0.04% trifluoroacetic acid. Approximately 1.8 mg of material was isolated. Final purification of compound C2 used an isocratic method with 58% water and 42% acetonitrile buffered with 0.04% trifluoroacetic acid. Approximately 1.5 mg of material was isolated. The UV maxima and ESI-MS molecular ion information (doubly-charged ions observed in negative ion mode) for compound C1 and compound C2 are presented below:

| | Compound C1 | Compound C2 |
|---|---|---|
| ESI-MS (m/z) | 814 (M-2H)²⁻ | 821 (M-2H)²⁻ |
| UV-vis ?ₘₐₓ/nm | 221, 280, | 221, 280 |

### EXAMPLE 2-6

### Module Exchaniges Constructed at Positions 8 and 11 in DptBC

A plasmid carrying dptBC pKN24 was constructed by truncation of B12:03 A05 that carries daptomycin biosynthetic (dpt) gene cluster. The Red-mediated recombination system was employed to introduce linear PCR products of antibiotic resistance genes flanked by 45 bp sequences with homology to either upstream or downstream regions of the interested dpt genes (as described in Example 2-4). The upstream (5′) region of dptBC (pKN24-26) or dptD (pKN27) was deleted by the spec-ermEp* cassette that contains a spectinomycin resistant gene (spec) and strong, constitutively expressed ermEp*. This fragment was amplified using the primers Sp6Del-1-2 and dptBC-ermEp. The downstream (3′) region of dptBC (pKN24) was deleted by a beta-lactamase gene (amp, from pBR322). this fragment was amplified using primers GTC del2 and DptD-3′::amp.

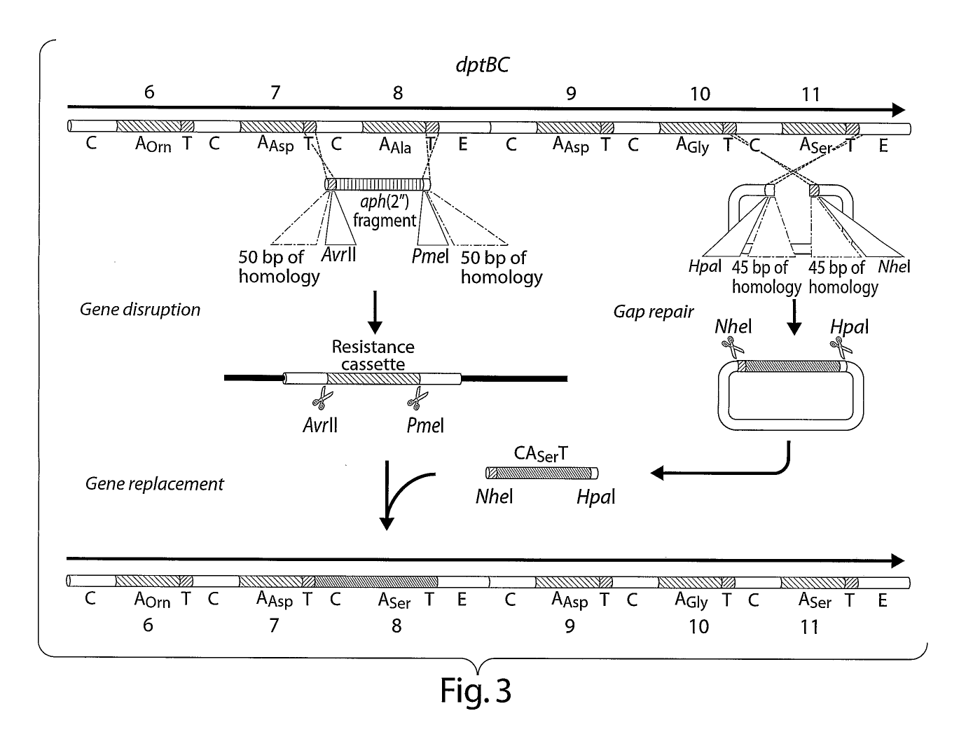

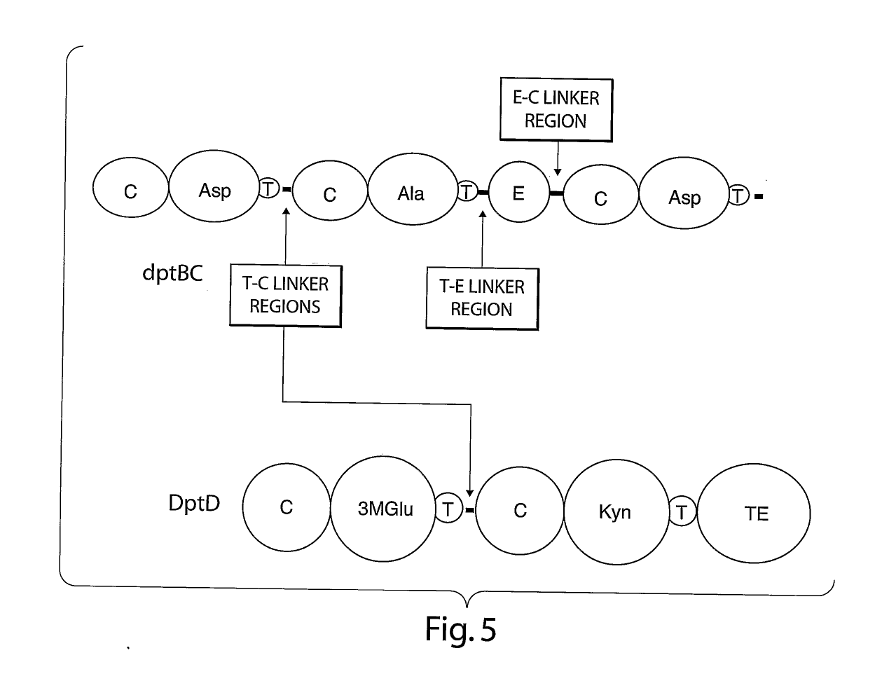

The selection cassette for the deletion of the CAT module was amplified with PCR primers that carry 50 bp of homology to the linker region of the module under investigation (see FIG. 5 for positions of linkers; International Patent Application Number WO 01/30985). When these PCR fragments were introduced into electro-competent cells that contained pKN24 (a truncated version of pDA300 that contains only dptBC, which is expressed from the constitutive promoter ermEp*, Bibb, M J. et al., 1985, Gene 38(1-3): 215-26) and induced Red-system, the resistance cassette was integrated site specifically at the target site in pDA300 by homologous recombination (FIG. 3).

5′ deletion, 3′ deletion
Sp6Del-1-2

(SEQ ID NO: 5)

5′-GCCAGCATGGAGCCGAACTGCCGGAACACCGCGTCCCGGTCCACCTG
TGTAGGCTGGAGCTGCTTC-3′,
GTC del2

(SEQ ID NO: 6)

5′-GCCGACTGGGAGTGGGTCAAGTGGCTGCCGCACGTGCTGGATCCGCA
TATGAATATCCTCCTTA-3′
dptBC-ermEp

(SEQ ID NO: 7)

5′-CCGAGACAGGCAGGATCTCCTCGACTACCTTCGACCGGCGGTTCATA
TG TCCGCCTCCTTTGGTCAC-3′,
DptD-3′::amp

(SEQ ID NO: 8)

5′-CATACTTCCTCTCACTCCGCTGCAGGAGGGACTGCTGTTCCACAGTG
TGTAGGCTGGAGCTGCTTC-3′

These cells were then selected for the presence of the tet resistance marker, and the resulting colonies were analyzed genetically to validate the construction of the appropriate deletion or disruption. Part of the primer design involves placing a restriction site within the linker region of interest (FIG. 3). Once the deletion BAC was verified, the selection cassette was excised using the unique restriction sites incorporated into the linker regions (FIG. 3 AvrII and PmeI).

The replacement modules (Serine; Alanine), were subcloned into pBR322 (Yanisch-Perron et al. 1985, Gene 33(1)-103-19; flanked by appropriate sites) again using the Red-mediated recombination. This technique is referred to as gap-filling, where the primers include the 50 bp overlap with the regions inside the linkers of the desired module (Lee, E C. et al., 2001, Genomics 73: 56). The primers were used to amplify a part of pBR322, including the origin of replication and apr to generate a linear fragment flanked by the regions of homology inside the desired module (Serine; Alanine). These PCR fragments were introduced into DH10B electro-competent E. coli cells containing pKN24 (see above) and pKD78 (Datsenko, K A., and Wanner, B L., 2000, Proc. Nat. Acad Sci U.S.A. 97: 6640). Once recombination has occurred through both regions of homology the module will have been transferred from the original vector to pBR322, converting the linear PCR fragment into a circular version that can replicate and be selected for (FIG. 3). It is preferred if the original vector that the module is cloned from has an F-plasmid origin of replication (as opposed to an origin of replication with a higher copy number).

The cloned modules are excised from pBR322 and ligated into the deleted versions of pKN24 using the compatible restriction sites introduced around the deletion. This produced 2 plasmids: 1) pDR2155 where the D-serine-11 of daptomycin had been replaced by D-alanine by module exchanges and 2) pDR2160 where D-alanine-8 of daptomycin had been replaced by D-serine. Both pDR2155 and pDR2160 were confirmed via PCR and sequencing.

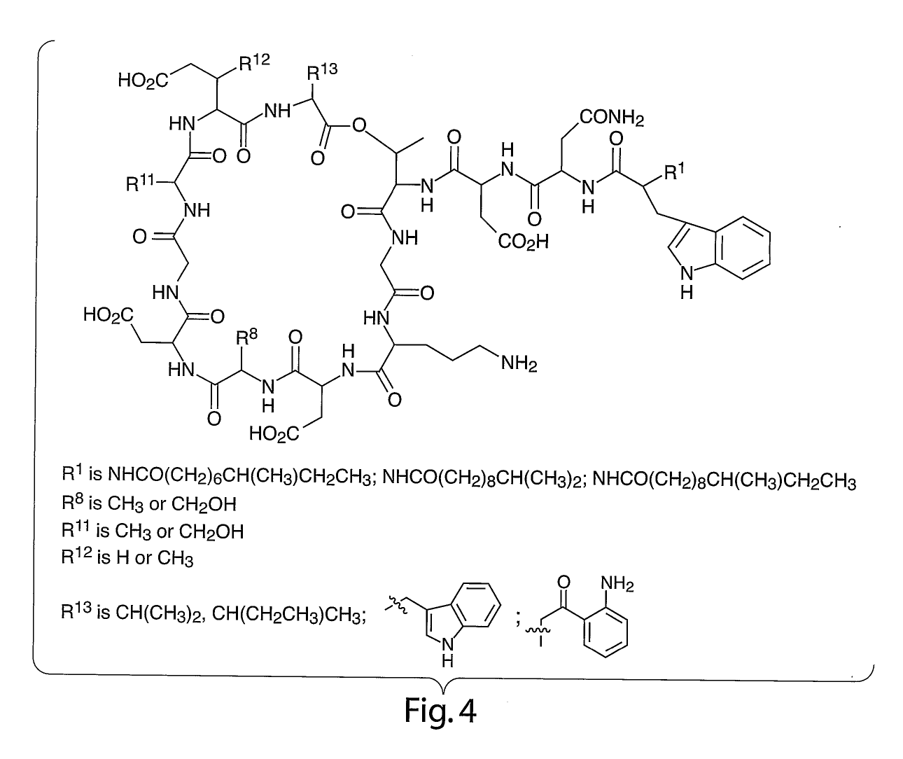

A suitable expression host was then constructed in S. roseosporus for these plasmids. A dptB-D mutant KN100 which contains a chromosomal deletion that removes dptBC, D was constructed using the techniques described in Example 2-1. Both pKN24 and pRB04 (a plasmid constructed in the vector pHM11a which expresses the dptD subunit under the control of eimnE* constitutive promoter) were added by interspecies conjugation to KN100 strains to create KN101. When fermented and analyzed under the conditions described in Examples 2-2 and 2-3, this strain was shown to produce the native lipopeptides A21978C, A21978C₂ and A21978C₃. Once the S. roseosporus KN100 strain had been validated, then a second derivative was created, KN156 (KN100 carrying pRB04). This strain was then used as the host for all module exchanges performed in dptBC. PB103 was constructed by addingpDR2155 to KN156. When fermented and analyzed under the conditions described in Examples 2-2 and 2-3, this strain was shown to produce compounds C46, C47 and C48 FIG. 4).

StrainPB118 was constructed by adding pDR2160 to KN156. When fermented and analyzed under the conditions described in Examples 2-2 and 2-3, this strain was shown to produce compounds C22, C23 and C24 (FIG. 4).

The recombinant strain described above, were fermented and analyzed under the conditions described in Example 2-2 and then analyzed using the techniques described in Example 2-3 (The data is summarized in Table VI).

Derivatives having Asn at the position 8 or 11 were prepared by module exchange using fusion sites TC (B) and TE (CAT). The Red-mediated recombination system was used to replace module 8 or 11 on pKN24 by gentamycin resistance gene (ahp2) (Chow J W, Kak V, You I, Kao S J, Petrin J, Clewell D B, Lerner S A, Miller G H, Shaw K J. 2001, Antimicrob. Agents Chemother. 45, 2691-2694) flanked by engineered AvrII and PmeI restriction sites. Since the DNA sequences of the module 8 and 11 are highly homologous, the same primer pair was used for deletion of the two modules at the linkers B and CAT.

A DNA fragment coding for an Asn module (B-CAT), the 11th module from A54145 NRPS was cloned by the gap-repair method. Gap-repair primers were used for PCR amplification of a portion of pBR322 including amp resistance gene and origin of replication to generate a linear fragment flanked by incorporated NheI and HpaI restriction sites and 45 bp with homology inside the desired module fragments. The linear PCR fragment was transformed into electro-competent E. coli carrying SF1:10D08 (a BAC clone of >100 kb DNA encoding parts of the A54145 biosynthetic gene cluster. (This clone was derived from a genomic BAC-based library of S. fradiae that was constructed using the protocols described in Miao et al., 2005, Microbiology 151:1507-1523. Clone BAC-P13 was isolated from the library using the protocols in Miao et al., 2005, Microbiology 151: 1507-1523) and tetR— pKD119 (Datsenko, K A., and Wanner, B L., 2000, Proc. Nat. Acad Sci U.S.A. 97: 6640) coding for the Red recombination system.) Once the Red-induced recombination occurs at both homologous regions, the Asn module was transferred from BAC-P13 to the linear pBR322 to generate a circular and replicated plasmid. Module fragments with correct sequences (as verified by sequencing) were excised by NheI and HpaI digestion and used for ligation with appropriate deleted pKN24 versions to generate hybrid plasmids.

Primers for deletion of module 8
pKN24-Mod8::Gen. B-CAT
8_B

(SEQ ID NO: 9)

TTGTTCGAGGCGCCGACGGTGAGCCGTTTGGAGCGGTTGCTGCGGGAGCG
CCTAGGACGTTGACACCATCGAATGG.
8_CAT-Pme

(SEQ ID NO: 10)

ACAATCTCAGCACCCCCCACCACACCAACCGCCCCAGCGTCCGAACCACG
TTTAAACCCTCATTCATCGGGCGAAAG
Primers for deletion of module 11
pKN24-Mod11::Gen. B-CAT
8_B

(SEQ ID NO: 11)

TTGTTCGAGGCGCCGACGGTGAGCCGTTTGGAGCGGTTGCTGCGGGAGCG
CCTAGGACGTTGACACCATCGAATGG
8_CAT-Pme

(SEQ ID NO: 12)

ACAATCTCAGCACCCCCCACCACACCAACCGCCCCAGCGTCCGAACCACG
TTTAAACCCTCATTCATCGGGCGAAAG
Primers for gap-repair of lptAsn11.
Lpt-N11-B-P13

(SEQ ID NO: 13)

TCGGGGCGCGGGTCGGCGGGGCGCAGCCGGGGTCCGGCCTCGCCC
GCTAGCTTCTTAGACGTCAGGTGGCAC
Lpt-N11-CAT-P14

(SEQ ID NO: 14)

CGCGACATCTTCGAACAGCGCACGCCCGCCGCCCTCGCCGGCCGC
GTTAACCGATACGCGAGCGAACGTGA

Plasmids were screened by PCR and restriction digests, plasmids with the correct genotype were then designated as pKN45 (D-Asn module inserted at position 8) and pKN47 (D-Asn module inserted at position 11). These two plasmids were then conjugated into the expression host KN156 (ΔdptBCD+pRB04). Exconjugants were selected on ASI plates containing apramycin (50 μg/mL) and recombinant strains selected from these plates were then fermented and analyzed using the protocols described in Example 2-2 and 2-3. Novel lipopeptides C189, C190 and C191 with molecular weights consistent with the insertion of Asn at position 8 in A21978C_{1,2,3} were detected by LC-MS from the fermentation broth of KN392 (see table VI). Novel lipopeptides C233, C234 and C235 with molecular weights consistent with the insertion of Asn at position 11 in A21978C_{1,2,3} were detected by LC-MS from the fermentation broth of KN404(see table VI).

TABLE VI
Data from module exchanges at position 8, 11

| | Replacement | | | |
|---|---|---|---|---|
| Dpt | amino acid | | | |
| amino | (source | 5′ | 3′ | |
| acid # | pathway) | linker | linker | Results |
| D-Ala-8 | D-Ser (dpt) | T-C^{#} | T-E^{#} | Lipopeptide with molecular |
| | | | | mass of 1650 (compound |
| | | | | C22), 1664 (compound |
| | | | | C23) and 1678 (compound |
| | | | | C24) detected. |
| D-Asn-8 | D-Ser (dpt) | T-C^{#} | T-E^{#} | Lipopeptide with molecular |
| | | | | mass of 1677.72 (compound |
| | | | | C189), 1691.75 (compound |
| | | | | C190) and 1705.78 |
| | | | | (compound C191) detected. |
| D-Ser-11 | Ala (dpt) | T-C^{#} | T-E^{#} | Lipopeptide with molecular |
| | | | | mass of 1618 (compound |
| | | | | C46), 1632 (compound |
| | | | | C47) and 1646 (compound |
| | | | | C48) detected. |
| D-Asn-11 | Ala (dpt) | T-C^{#} | T-E^{#} | Lipopeptide with molecular |
| | | | | mass of 1661.72 (compound |
| | | | | C243), 1675.75 (compound |
| | | | | C244) and 1689.78 |
| | | | | (compound C245) detected. |

(^{#}See FIG. 5 for positions of T-C and T-E)

Once the presence of the expected mass ions was confirmed PB103, PB118, KN392 and KN404 were fermented at large scale and compounds C22, C46, C189, C233 were purified using the techniques described in Example 2.5.

### EXAMPLE 2-7

### Module Exchanges at Position 13 in dptD

Module exchanges were constructed at position 13 in the dpt cluster to replace kynurenine. These constructs were made in the subunit expression plasmid pRB04 (a plasmid constructed in the vector pHM11a which expresses the dptD subunit under the control of ermE* constitutive promoter described in Example 2-5). A unique AvrII site was introduced inside the T-C linker. A second unique PmeI was introduced just downstream of the coding region of dptD. This allowed the terminal module for Kyn to be removed from dptD along with the thioesterase. Two replacement modules containing the domain arrangement CATTe were prepared as fragments flanked by AvrII and PmeI sites. The isoleucine and tryptophan modules were responsible for the incorporation of the terminal amino acids in the A54145 (Ile) and CDA (Trp) pathways. After cloning the replacement modules into the deleted pRB04 the hybrid constructs were introduced into a dptD deleted S. roseosporus, and fermentation and analysis were completed using the techniques described in Example 2-1. This data is summarized in Table VII.

TABLE VII
Data from module exchanges at position 13

| Replacement | | | | |
|---|---|---|---|---|
| amino acid | Dpt | | | |
| (source | amino | 5′ | 3′ | |
| pathway) | acid # | linker | linker | Results |
| Trp (CDA) | Kyn-13 | T-C^{#} | 3′ of | Lipopeptide with molecular mass |
| | | | dptD | of 1630 (compound C1), 1644 |
| | | | | (compound C2) and 1658 |
| | | | | (compound C3) detected. |
| Ile (A54145) | Kyn-13 | T-C^{#} | 3′ of | Lipopeptide with molecular mass |
| | | | dptD | of 1557 (compound C4), 1571 |
| | | | | (compound C5) and 1585 |
| | | | | (compound C6) detected. |

^{#}Linkers are defined in FIG. 5

### EXAMPLE 2-8

### Deleting the dptI Gene from Daptomycin NRPS Gene Cluster Results in the Production of Lipopeptides with Glutamate at Position 12

Sequence comparisons between the dptI, lptI and glmT genes suggested that dptI may play a role in the methylation of the glutamate in position 12 (the glmT gene product is believed to methylate the glutamate in a similar position in the related lipopeptide CDA; the lptI gene product is believed to methylate glutamate in the synthesis of A54145). To test this theory a deletion was created in the dptI gene in S. roseosporus UA431 containing pDA300. A deletion plasmid was constructed that contained 2x1kb fragments that flanked dptI upstream and downstream. These fragments were ligated in such a way that they would create an in-frame deletion of dptI. This cassette was cloned into pRHB538 (see Example 2-1) and introduced in S. roseosporus UA431/pDA300. Under the appropriate selection conditions (see Example 2-1) the deletion cassette was exchanged for the dptI gene on the chromosome, thus constructing an in-frame deletion of dptI. The genotype of this mutant was confirmed by PCR and Southern blots. This mutant was fermented and analyzed using the techniques described in Example 2-1. The results of this analysis were that these strains were only able to produce lipopeptides with masses of 1620, 1634 and 1648, which corresponded to the predicted masses for lipopeptides that contain glutamate at position 12 instead of 3-methyl-glutamate: compound C10, compound C11 and compound C12 respectively. From this data it was concluded that dptI plays a role in the methylation of glutamate during the synthesis of daptomycin.

### EXAMPLE 2-9

### Construction of a Combinatorial Library of Novel Lipopeptides from Recombinant Streptomyces Roseosporus

Successful module exchanges produced from Example 2-5 were further enhanced by combining pDR2155 and pDR2160 with subunits exchanges for dptD that could include lptD (the terminal subunit from the S. fradiae A54145 biosynthetic pathway that encodes for 3MeGlu and Ile/Val cloned in an expression plasmid, supra) and cdaPS3 (the terminal subunit from the S. coelicolor calcium dependent antibiotic, CDA, biosynthetic pathway that encodes for 3MeGlu and Trp cloned in an expression plasmid, supra). These combinations were further enhanced by being expressed in hosts that contain a dptI (a putative methyl-transferase involved in the methylation of glutamate at position 12 of daptomycin) deletion which will lead to the inclusion of glutamate at position 12 instead of 3-methyl-glutamate. One or more of the methods described above:

1. module exchanges to effect alterations at positions 8 and 11,

2. dptI deletion to effect alterations at position 12, and

3. subunit complementation to effect alterations at position 13 were combined to construct combinatorial libraries that contained 48 novel lipopeptides.

In addition to the construction of KN100 described in Example 2-5a second S. roseosporus mutant was constructed, designated KN125 (using the techniques described in Example 2-1) that contained a chromosomal deletion that removes dptBC, D, G, H, I, J. After KN125 was confirmed as a null mutant it was used to construct KN159 by adding pKN24 and pRB04 to KN125. When fermented and analyzed under the conditions described in Examples 2-2 and 2-3, this strain was shown to produce compounds C10, C11 and C12 which all lack the methyl group on glutamate 12 seen in A21978C.

Strain KN107 was constructed by adding pKN24 and pMF23 to KN100. When fermented and analyzed under the conditions described in Examples 2-2 and 2-3, this strain was shown to produce compounds C1, C2 and C3.

Strain KN110 was constructed by adding pKN24 and pMF30 to KN100. When fermented and analyzed under the conditions described in Examples 2-2 and 2-3, this strain was shown to produce compounds C4, C5, C6, C7, C8, and C9.

Strain KN160 was constructed by adding pKN24 and pMF23 to KN125. When fermented and analyzed under the conditions described in Examples 2-2 and 2-3, this strain was shown to produce compounds C13, C14 and C15.

Strain KN161 was constructed by adding pKN24 and pMF30 to KN125. When fermented and analyzed under the conditions described in Examples 2-2 and 2-3, this strain was shown to produce compounds C16, C17, C18, C19, C20, and C21.

The combinatorial approach described above, was then enhanced by the addition of the modified dptBC constructs in pDR155 and pDR2160. Strain PB105 was constructed by adding pDR2155 and pMF23 to KN100. When fermented and analyzed under the conditions described in Examples 2-2 and 2-3, this strain was shown to produce compounds C49, C50 and C51.

Strain PB108 was constructed by adding pDR2155 and pMF30 to KN100. When fermented and analyzed under the conditions described in Examples 2-2 and 2-3, this strain was shown to produce compounds C52, C53, C54, C55, C56, and C57.

Strain PB110 was constructed by adding pDR2155 and pRB04 to KN125. When fermented and analyzed under the conditions described in Examples 2-2 and 2-3, this strain was shown to produce compounds C58, C59 and C60.

Strain PB113 was constructed by adding pDR2155 and pMF23 to KN125. When fermented and analyzed under the conditions described in Examples 2-2 and 2-3, this strain was shown to produce compounds C61, C62 and C63.

Strain PB116 was constructed by adding pDR2155 and pMF30 to KN125. When fermented and analyzed under the conditions described in Examples 2-2 and 2-3, this strain was shown to produce compounds C64, C65, C66, C67, C68, and C69.

Strain PB120 was constructed by adding pDR2160 and pMF23 to KN100. When fermented and analyzed under the conditions described in Examples 2-2 and 2-3, this strain was shown to produce compounds C25, C26 and C27.

Strain PB123 was constructed by adding pDR2160 and pMF30 to KN100. When fermented and analyzed under the conditions described in Examples 2-2 and 2-3, this strain was shown to produce compounds C28, C29, C30, C31, C32, and C33.

Strain PB128 was constructed by adding pDR160 and pRB04 to KN125. When fermented and analyzed under the conditions described in Examples 2-2 and 2-3, this strain was shown to produce compounds C34, C35 and C36.

Strain PB130 was constructed by adding pDR2160 and pMF23 to KN125. When fermented and analyzed under the conditions described in Examples 2-2 and 2-3, this strain was shown to produce compounds C37, C38 and C39.

Strain PB131 was constructed by adding pDR2160 and pMF30 to KN125. When fermented and analyzed under the conditions described in Examples 2-2 and 2-3, this strain was shown to produce compounds C40, C41, C42, C43, C44, and C45

Strain KN393 was constructed by adding pKN45 and pMF23 to KN100. When fermented and analyzed under the conditions described in Examples 2-2 and 2-3, this strain was shown to produce compounds C198, C199 and C200.

Strain KN394 was constructed by adding pKN45 and pMF30 to KN100. When fermented and analyzed under the conditions described in Examples 2-2 and 2-3, this strain was shown to produce compounds C201, C202, C203, C210, C211, and C212.

Strain KN395 was constructed by adding pKN45 and pRB04 to KN125. When fermented and analyzed under the conditions described in Examples 2-2 and 2-3, this strain was shown to produce compounds C192 C193 and C194.

Strain KN396 was constructed by adding pKN45 and pMF23 to KN125. When fermented and analyzed under the conditions described in Examples 2-2 and 2-3, this strain was shown to produce compounds C195, C196 and C197.

Strain KN397 was constructed by adding pKN45 and pMF30 to KN125. When fermented and analyzed under the conditions described in Examples 2-2 and 2-3, this strain was shown to produce compounds C204, C205, C206, C207, C208, and C209.

Strain KN405 was constructed by adding pKN47 and pMF23 to KN100. When fermented and analyzed under the conditions described in Examples 2-2 and 2-3, this strain was shown to produce compounds C224, C225 and C226.

Strain KN406 was constructed by adding pKN47 and pMF30 to KN100. When fermented and analyzed under the conditions described in Examples 2-2 and 2-3, this strain was shown to produce compounds C221, C222, C223, C213, C214, and C215.

Strain KN407 was constructed by adding pKN47 and pRB04 to KN125. When fermented and analyzed under the conditions described in Examples 2-2 and 2-3, this strain was shown to produce compounds C230, C231 and C232.

Strain KN408 was constructed by adding pKN47 and pMF23 to KN125. When fermented and analyzed under the conditions described in Examples 2-2 and 2-3, this strain was shown to produce compounds C227, C228 and C229.

Strain KN409 was constructed by adding pKN47 and pMF30 to KN125. When fermented and analyzed under the conditions described in Examples 2-2 and 2-3, this strain was shown to produce compounds C72, C219, C220, C216, C217, and C218

### EXAMPLE 2-10 MODULE EXCHANGES CONSTRUCTED AT POSITIONS 2 THROUGH 4 in Daptomycin

Multiple module exchanges were performed in either dptA, dptBC or a combination of dptA and BC in order to complete these exchanges a new expression plasmid was needed as pKN24 only contained dptBC. The new vector, pKN18, was a truncated product of B12:03A05 (a BAC clone that contains entire daptomycin biosynthetic pathway, see Example 2-1) that was able to express both dptA and dptBC. Plasmid pKN18 was constructed by truncating B12:03A05 using the Red-mediated recombination (see Example 2-5) system through two sequential deletions of B12:03A05. The two deletions deleted all of genes upstream of dptR and all of the genes downstream of dptBC (pKN18 carries the locus dptR-drrAB-dptEFABC). Firstly, the upstream (5′) region of the locus (insert coordinate 0.552 kb-45,576 kb on B12:03A05, see table VI for primers) was deleted by spectinomycin resistance gene. The region downstream (3′) of dptBC (insert coordinate 91,093 kb-127,392 kb, see table V for primers) was deleted by amp gene.

In order to combine module exchanges in dptA,BC with subunit swaps for dptD (see Example 2-6) and peptide tailoring methyl transferase dptI (see Example 2-8) it was necessary to construct a new expression plasmid that could express the dptIJ genes in the dpt deletion host UA431 (see Example 2-1) with the modified pKN18 plasmids. In order to express a glutamate methyltransferase in UA431 (ΔdptE-J), pKN54, a plasmid that carries strong promoter permEp* and functions for integration on chromosome from phi-BT1 phage was constructed based on kanR pRT802 (Gregory, M. A.; Till, R.; Smith, M. C.; 2003. J. Bacteriol 185: 5320-5323.). The 1.8 kb BglII/SmaI fragment from pHM11a which carries ermEp* and a transcriptional terminator (.Integrative vectors for heterologous gene expression in Streptomyces spp. Motamedi, H; Shafiee, A; Cai, S J; 1995, Gene., 160: 25-31) was cloned at BamHI/EcoRV sites of pRT802 (Gregory, M. A.; Till, R.; Smith, M. C.; 2003. J. Bacteriol. 185: 5320-5323), which encodes for phi-BT1 integration system. The plasmid was multiplied in selective medium with kanamycin (50 μg/mL).

A DNA fragment coding for both dptI and dptJ was PCR amplified using B12:03A05 as the template. Two primers (with engineered restriction sites underlined) dptJ-C-HindIII: 5′-GGCGGAAGCTTACGGCACGGCAAGGCCGTTTC-3′ (SEQ ID NO: 15) and dptI-N-NdeI: 5′-GGCGGCATATGACCGTGCACGACTACCAC-3′ (SEQ ID NO: 16) were used for the PCR amplification. The PCR fragment was cloned on pKN54 at NdeI and HindIII sites to generate pKN55.

Finally, a series of expression hosts were created that would be used for the multi-modular exchanges described in this Example. KN576 was constructed by introducing pRB04 (expresses dptD from minicircle integration sites, see Example 2-6) into UA431 (ΔdptE-J). KN580 was constructed by introducing pRB04 (see Example 2-6) and pKN55 (dptIJ expressed from phi-BT1 integration sites) into UA431 (ΔdptE-J). KN577 was constructed by introducing pMF30 (expresses lptD from minicircle integration sites, see Example 2-6) into UA431 (ΔdptE-J). KN587 was constructed by introducing pMF30 (see Example 2-6) and pKN55 into UA431 (ΔdptE-J).

Sp6 del3

(SEQ ID NO: 17)

5′GCATCCGATGCAAGTGTGTCGCTGTCGACGGTGACCCTATAGTCGTGT
AGGCTGGAGCTGCTTC
Sp6 del4

(SEQ ID NO: 18)

5′-CCGAGGAAAAGAGGGAACGGGACAGGTCAGTGACCGGCGACCGTGCA
TATGAATATCCTCCTTA-3′
DptD-3′::amp

(SEQ ID NO: 19)

5′-CATACTTCCTCTCACTCCGCTGCAGGAGGGACTGCTGTTCCACAGTG
TGTAGGCTGGAGCTGCTTC-3′
GTC del2

(SEQ ID NO: 20)

5′-GCCGACTGGGAGTGGGTCAAGTGGCTGCCGCACGTGCTGGATCCGCA
TATGAATATCCTCCTTA-3′

Multi module exchanges were completed on pKN18 using the Red-mediated recombination system to change several amino acid residues on the daptomycin core simultaneously. First, the genR (Wohlleben, W. et al., 1989, Mol. Gen. Genet. 217: 202-208) gene was introduced into pKN18 to replace the DNA fragment coding for modules 2-3-4 (2-4), between the linker regions B and CAT (exchanges 2-4). The genR gene was then removed by AvrII/PmeI digest.

DNA fragments coding for modules 2-4, from the A54145 pathway were cloned onto pBR322 by the gap-repair method as described for single module exchange in Example 2-5. This fragment was excised by NheI and HpaI digests and ligated to the deleted pKN18 to generate pKN51 (carries D-Glu at position 2 and Asn at position 3 in daptomycin). This plasmid, pKN51, was introduced into expression hosts: KN576 to produce KN630, KN580 to produce KN631, KN577 to produce KN632 and KN587 to produce KN633 via recombination. These recombinant strains were fermented and analyzed using the techniques described in Example 2-2 and 2-3. The fermentation broth of KN633 was the only strain to contain mass ions consistent with the production of C259, C260, C261, C262, C263 and C264. LC/MS analysis of the fermentation broths from the other strains KN630, KN631 and KN632 did not reveal the presence of any novel lipopeptides.

Primers for deletion of
dpt2-4.
dpt-Asn2-Del-B:

(SEQ ID NO: 21)

GTTCGCCTTCCCCACCGTCGCCGGCCTTCTCCCGCTCCTGGACGACAA
CCTAGGTGTGTAGGCTGGAGCTGCTTCG
dpt-Thr4-Del-CAT:

(SEQ ID NO: 22)

TCAGGGCGCCGGTCGATCCTGGTCACAGGTGGCAGGGCGGTGCCGG
GTTTAAACCATATGAATATCCTCCTTA
Primers for gap repair cloning
lpt2-4
LptGlu2-Pickup-B:

(SEQ ID NO: 23)

5′ TCC GGG CGG GGC CGG ACG GGA CGG ACG TGG TCG TCC
GGC ACG GCC GCTAGCTTCTTAGACGTCAGGTGGCAC 3′
lpt-Thr4-pickup-CAT:

(SEQ ID NO: 24)

5′ TTC GAG GCG CCC ACG CCC GCC GCG CTG TCC CGG CGC
CTC GACACCGTTAAC CGATACGCGAGCGAACGTGA 3′

### EXAMPLE 2-11 MODULE EXCHANGES CONSTRUCTED AT POSITIONS 8 THROUGH 11 in Daptomycin

A daptomycin derivative containing 2 changes at positions 8 and 11 was generated using the Red-mediated recombination system as described in Example 2-5. Briefly, a DNA fragment coding for 4 modules (D-Ala8-Asp9-Gly10-D-Serl 1) was deleted from pKN24 by a gentamycin resistance gene franked by AvrII and PmeI restriction sites. The genR gene was then removed by AvrII/PmeI digest.

The corresponding DNA fragment coding for module 8-9-10-11 (D-Lys-Asp-Gly-D-Asn) from A54145 BAC-P13 that was subcloned on pBR322 by the gap-repair method (Example 2-5) was used for ligation with the deleted pKN24 to generate pKN50. pKN50 was introduced into KN156 (see Example 2-9, S. roseosporus ΔdptBC,D+pRB04 [a plasmid constructed in the vector pHM11a which expresses the dptD subunit under the control of ermE* constitutive promote, see Example 2-5]) to create KN410. KN410 was fermented and analyzed using the protocols described in Examples 2-2 and 2-3. Analysis of the LC/MS data showed the presence of mass ions that were consistent with the insertion of lysine at position 8 and asparagines at position 11 of A21978C_{1,2,3}. These compounds were designated C236, C237, C238.

Primers for deletion of module 8-11
8_B

(SEQ ID NO: 25)

TTGTTCGAGGCGCCGACGGTGAGCCGTTTGGAGCGGTTGCTGCGGGAGCG
CCTAGGACGTTGACACCATCGAATGG
11_SUE (deletion ended at the 3′ terminus
of dptBC)

(SEQ ID NO: 26)

CAGCTCGCTGATGATATGCTGACGCTCAATGCCGTTTGGCCTCCGACTAA
GTTTAAACCCTCATTCATCGGGCGAAAG
Primers for gap repair of module 8-11
lptK8-B-P13

(SEQ ID NO: 27)

GTCCTCCGACCGCGACATCCGTCGCAACGCGGGGCGGGTGTCAGGGCGG
GCTAGCTTCTTAGACGTCAGGTGGCAC
lpt-N11-SUE-P14 (cloned fragment extended to the
3′ terminus of lptBC)

(SEQ ID NO: 28)

CACCGAACTCGACCAGCTCGAAGCAGAGTGGAAGGCCGGCTGATG
GTTAACCGATACGCGAGCGAACGTGA

### EXAMPLE 2-12

### Construction of an S. Fradiae-Based in-Trans Expression System for the Production of Novel Lipopeptides

For the expression of the hybrid non-ribosomal polypeptide synthetase (NRPS) pathways, a version of the S. fradiae high A54145 factor-producing strain that lacked all of the NRPS and potential amino acid modification genes was constructed. Engineered modified pathways were conjugated into this strain on BAC-based vectors which integrated site-specifically in a neutral site of the S. fradiae genome at a φ31 attB site.

To delete all the proposed NRPS genes from S. fradiae a deletion cassette was constructed that contained flanking DNA from upstream of lptEF and downstream of lptI.

Flanking regions from upstream of lptEF (5′) and downstream of lptI (3′) were cloned around a selection cassette containing tsr and cat. The 5′ fragment was 3665 bp long and the 3′ fragment was 2004 bp long. These two fragments were cloned together with the tsr and cat resistance cassettes into a copy of the delivery plasmid called pRHB538 (Hosted, T J. and Baltz, R H., 1997, J. Bacteriol. 179(1): 180-6), which contains a temperature sensitive origin of replication and a dominant allele of rpsL (streptomycin sensitive). This plasmid was introduced into a S. fradiae strain carrying a recessive rpsL allele that confers streptomycin resistance. This recombinant strain was then incubated overnight in a broth culture at 39° C. before the cells were spread on plates containing streptomycin plus thiostrepton and incubated at 39° C. Under these conditions only those strains that have exchanged the deletion cassette (containing tsr and cat) for the lptE-I locus via homologous recombination survived the selection; all other genotypes were eliminated. This strain was then designated S. fradiae DA1187.

### EXAMPLE 2-13

### Fermenting S. Fradiae Strains

Mycelial glycerol stocks of the S. fradiae DA1187 stored at −80° C. were plated onto agar plates of medium R [10.3% sucrose (Sigma), 0.025% potassium sulfate (Sigma), 1.01% magnesium chloride hexahydrate (Sigma), 1% glucose (Sigma), 0.01% casamino acids (Difco), 0.5% yeast extract (Difco), 0.57% TES buffer (Sigma), 2.2% agar (MBI), 0.005% potassium phosphate (Sigma), 0.29% calcium chloride dihydrate (Sigma) and 0.07% sodium hydroxide (Sigma)] (see Kieser) and grown for 3-5 days at 30° C.

A starter culture was generated by gently macerating material from the agar plate surface to generate a suspension of vegetative mycelium and spores which was added to 8 ml of C medium [3% trypticase soy broth (Difco), 0.3% yeast extract (Difco), 0.2% magnesium sulfate (Sigma), 0.5% glucose and 0.4% maltose (Sigma), Hosted, T J., and Baltz, R H., 1996, Microbiology 142: 2803-2813] in a 50 ml culture tube with appropriate antibiotics. Starter cultures were shaken at 240 rpm for 24 to 36 hours at 30° C.

A one mL aliquot of this culture was transferred into a 125 mL baffled flask containing 25 mL of nutrient solution S (1% D-glucose (BDH), 1.5% glycerol (BDH), 1.5% soya peptone (Sigma), 0.3% sodium chloride (BDH), 0.5% malt extract (Oxoid), 0.5% yeast extract (Lab M), 0.1% Junlon PW100 (Honeywell and Stein Ltd), 0.1% Tween 80 (Sigma), 4.6% MOPS (Sigma) adjusted to pH 7.0 and autoclaved)) and shaken at 200 rpm for 24 to 36 hours at 30° C.

Production cultures were generated by aseptically transferring 5% of the seed culture to baffled 250 mL flasks containing 50 mL medium D (3% glucose (Sigma), 2.5% soybean flour (Arkady), 0.5% blackstrap molasses (DSM Bakeries), 0.06% ferric ammonium sulfate (Sigma), 0.79% L-isoleucine (Sigma) and 6% calcium carbonate (Sigma) adjusted to pH 7 and autoclaved, (Boeck et al., 1990, J. Antibiotics 43: 607-615.) and shaken at 200 rpm for up to 7 days at 30° C. The addition of L-isoleucine to medium D had been shown to increase the proportion of factors with isoleucine at position 13 (Ilel 3) and decrease the proportion of factors with valine at position 13 (Val13) (Boeck et al., 1990, J. Antibiotics 43: 607-615.). On occasion, fermentations were done in medium D without isoleucine and these fermentation broths had a mix of both Ilel 3 and Val13 factors.

### EXAMPLE 2-14

### Analysis of the A54145 Lipopeptides from Fermentations of the Streptomyces Fradiae

Production cultures described in Example 2-2 were sampled for analysis by aseptically removing 2 mL of the whole culture and centrifuging for 10 minutes prior to analysis. Volumes up to 50 microlitres of the supernatant were analyzed to monitor for production of the native lipopeptides (A21978C) as produced by Streptomyces roseosporus. This analysis was performed at ambient temperature using a Waters Alliance 2690 HPLC system and a 996 PDA detector with a 4.6×50 mm Symmetry C8 3.5 μm column and a Phenomenex Security Guard C8 cartridge. The gradient initially holds at 90% water and 10% acetonitrile for 2.5 minutes, followed by a linear gradient over 6 minutes to 100% acetonitrile. The flow rate is 1.5 mL per minute and the gradient is buffered with 0.01% trifluoroacetic acid. By day 2 of the fermentation, production of three of the native lipopeptides, A21978C₁, A21978C₂ and A21978C₃, with UV/visible spectra identical to that of daptomycin, was evident, as shown by HPLC peaks with retention times of 5.62, 5.77 and 5.90 minutes (?max 223.8, 261.5 and 364.5 nm) under the analytical conditions stated. The lipopeptides then remained evident in the fermentation at each sample point during the 7-day period. Total yields of lipopeptides A21978C₁, A21978C₂ and A21978C₃ ranged from 10-20 mg per liter of fermentation material.

Liquid chromatography-mass spectrometry (LC-MS) analysis was performed on a Finnigan SSQ710c LC-MS system using electrospray ionization in positive ion mode, with a scan range of 200-2000 daltons and 2 second scans. Chromatographic separation was achieved on a Waters Symmetry C8 column (2.1×50 mm, 3.5 μm particle size) eluted with a linear water-acetonitrile gradient containing 0.01% formic acid, increasing from 10% to 100% acetonitrile over a period of six minutes after a initial delay of 0.5 minutes, then remaining at 100% acetonitrile for a further 3.5 minutes before re-equilibration. The flow rate was 0.35 mL/minute and the method was run at ambient temperature.

The identification of the native A54145 lipopeptides was confirmed in the controls (S. fradiae wild type grown in medium D without isoleucine), as indicated by molecular ions ([M+H]⁺) at m/z of 1630.7, 1644.7, 1644.7, 1658.7, 1658.7, 1658.7, 1672.7, which is in agreement with the masses reported for the major A54145 lipopeptide factors F, A, A1, B1, B, D, E, respectively, produced by Streptomyces fradiae (Boeck et al., 1990, J. Antibiotics 43: 587-593). The DA1187 mutants failed to produce any of the A54145 lipopeptides in medium D with or without isoleucine confirming that they were true null mutants.

### EXAMPLE 2-15

### Constructing pDA2002 and complementing the S. fradiae lptE-I deletion

Using the “Red” system the Streptomyces integrative BAC vector pDA2002 (that contains the lpt biosynthetic gene cluster) was constructed. This plasmid was constructed from SF1:1 OD08 (an E. coli BAC plasmid that contains all of the lpt biosynthetic gene cluster as well as flanking DNA, which was isolated from a chromosomal library of S. fradiae). The Streptomyces integrative cassette; containing the phiC31 integrase and attP site, the oriT from plasmid RK2, and the apramycin (apr) resistance marker, was engineered by DNA cloning to have flanking DNA regions with identity to the backbone of the BAC vector and orf21 of the S. fradiae insert. The Streptomyces integrative cassette was inserted into the BAC vector and a region of the BAC insert was deleted using homologous recombination via the Red-mediated recombination system. This was achieved by introducing SF1:10D08 into an E. coli strain carrying the Red genes on a plasmid (pKD119, Datsenko, K A., and Wanner, B L., 2000, Proc. Nat Acad Sci U.S.A. 97: 6640). This strain was then transformed with a gel purified fragment containing the Streptomyces integrative cassette flanked by appropriate described regions of homology. These cells were then selected for both apr and cat resistance, and the resulting colonies were analyzed genetically to validate the insertion of the integration cassette and deletion of sequences upstream of orf21. Once constructed the plasmid pDA2002 was introduced into S. fradiae DA1187 by conjugation to create strain DA1116. Plasmid pDA2002 contains oriT from plasmid RK2 (Baltz, 1998, Trends in Microbiol. 6: 76-83 (1998), incorporated herein by reference in its entirety) for conjugation from E. coli to S. fradiae. Plasmid pDA2002 is introduced into S. fradiae by conjugation from E. coli S17.1, or a strain containing a self-replicating plasmid RK2 (Id.). S. fradiae DA1116 was fermented and analyzed using the techniques described in Examples 2-13 and 2-14 respectively.

The identification of the native A54145 lipopeptides was confirmed, as indicated by molecular ions ([M+H]⁺) at m/z of 1630.7, 1644.7, 1644.7, 1658.7, 1658.7, 1658.7, 1672.7, which is in agreement with the masses reported for the major A54145 lipopeptide factors F, A, A1, B1, B, D, E, respectively, produced by Streptomyces fradiae when grown in medium D without isoleucine (Boeck et al., 1990, J. Antibiotics 43: 587-593). When grown in medium D with isoleucine the native A54145 lipopeptides with Ile13 predominated, as indicated by molecular ions ([M+H]⁺) at m/z of 1644.7, 1644.7, 1658.7, 1658.7, 1658.7, 1672.7, which is in agreement with the masses reported for the major A54145 lipopeptide factors A, A1, B1, B, D, E, respectively, This demonstrated that the pDA2002 was able to successfully complement the lptE-I deletion to restore lipopeptide production in DA 1116.

### EXAMPLE 2-16

### Removal of Downstream Cluster Genes by Insertion of Terminator Cassette to Identify Putative Amino Acid Modification Genes

The terminator cassette was engineered to place the to terminator from lambda phage in front of the amp resistance gene. The terminator cassette (to terminator plus amp) was amplified with PCR primers that carry 40 to 50 bp of homology to potential amino acid modification genes and the end of the BAC vector. When these PCR fragments were introduced into electro-competent cells that contained pDA002 and an induced Red-system, the terminator was integrated site specifically in pDA2002 by homologous recombination. These cells were then selected for the presence of the amp resistance marker as well as the apr resistance marker, and the resulting colonies were analyzed genetically to validate the insertion of the terminator cassette and deletion of sequences to the end of the BAC vector.

The terminator cassette was amplified with PCR primers that would insert the terminator downstream of orf46 to create pDA2080. pDA2080 was conjugated into S. fradiae DA1187 to create the strain DA1339. When fermented and analyzed under the conditions described in Examples 2-13 and 2-14, this strain was shown to produce lipopeptides with molecular ions ([M+H]⁺) at m/z of 1644.7, 1644.7, 1658.7, 1658.7, 1658.7, 1672.7, which is in agreement with the masses reported for the major A54145 lipopeptide factors A, A1, B1, B, D, E, respectively, produced by Streptomyces fradiae (Boeck et al., 1990, J. Antibiotics 43: 587-593). This demonstrated that the pDA2080 was able to successfully complement the lptE-I deletion to restore lipopeptide production in DA1339 even with the terminator cassette inserted into the BAC.

The terminator cassette was amplified with PCR primers that would insert the terminator into the lptI gene (putative methyltransferase of glutamate 12, see 2-8) to create pDA2054. pDA2054 was conjugated into S. fradiae DA1187 to create the strain DA1312. When fermented and analyzed under the conditions described in Examples 2-13 and 2-14, DA1312 was shown to produce lipopeptides with molecular ions ([M+H]⁺) at m/z of 1644.7, 1644.7, and 1658.7. This is in agreement with the masses reported for the major A54145 lipopeptide factors A, A1, and D, respectively, produced by Streptomyces fradiae that have glutamic acid (Glu12) instead of 3-methyl-glutamic acid (mGlu12) at position 12 (Boeck et al., 1990, J. Antibiotics 43: 587-593). From this data it was concluded that lptI plays a role in the methylation of glutamic acid during the synthesis of A54145.

The terminator cassette was amplified with PCR primers that would insert the terminator into the lptL gene (putative oxygenase of asparagine 3) to create pDA2076. pDA2076 was conjugated into S. fradiae DA1187 to create the strain DA1336. When fermented and analyzed under the conditions described in Examples 2-13 and 2-14, DA1336 was shown to produce lipopeptides with molecular ions ([M+H]⁺) at m/z of 1628.7, 1628.7, and 1642.7. This is consistent with the masses of analogs of the Glu12 factors A, A1, and D, respectively, that would have asparagine (Asn3) instead of 3-hydroxy-asparagine (hAsn3) at position 3. DA1336 was shown to produce the Ile13 compounds C93, C94, and C95, although not identified under these fermentation conditions this strain would also have the potential to produce the factors with Val13 C144, C145, and C146. From this data it was concluded that lptL plays a role in the addition of a hydroxyl group to the asparagine at position 3 during the synthesis of A54145.

The terminator cassette was amplified with PCR primers that would insert the terminator into the lptK gene (putative O-methyltransferase involved in the methoxylation of aspartic acid at position 9) to create pDA2074. pDA2074 was conjugated into S. fradiae DA1187 to create the strain DA1333. When fermented and analyzed under the conditions described in Examples 2-13 and 2-14, DA1333 was shown to produce lipopeptides with molecular ions ([M+H]⁺) at m/z of 1614.7, 1614.7, and 1628.7. This is consistent with the masses of analogs of the Glu12 factors A, A1, and D, respectively, that would have 3-hydroxy-aspartic acid (hAsp9) instead of 3-methoxy-aspartic acid (moAsp) at position 9 and Asn3 instead of hAsn3. DA1333 was shown to produce the Ile13 compounds C102, C103, and C104, although not identified under these fermentation conditions this strain would also have the potential to produce the factors with Val13 C132, C133, and C134. From this data it was concluded that lptK plays a role in the methoxylation of hydroxyl-aspartic acid at position 9 during the synthesis of A54145.

The terminator cassette was amplified with PCR primers that would insert the terminator into the lptJ gene (putative syrP regulator) to create pDA2060. pDA2060 was conjugated into S. fradiae DA1187 to create the strain DA1327. When fermented and analyzed under the conditions described in Examples 2-13 and 2-14, DA1327 was shown to produce lipopeptides with molecular ions ([M+H]⁺) at m/z of 1598.7, 1598.7 and 1612.7. This is consistent with the masses of analogs of the Glu12 factors A, A1, and D, respectively, that would have aspartic acid (Asp9) instead of moAsp9 at and Asn3 instead of hAsn3. DA1327 was shown to produce the Ile13 compounds C105, C106, and C107, although not identified under these fermentation conditions this strain would also have the potential to produce the factors with Val13 C135, C136, and C137. From this data it was concluded that lptJ is not a regulator of A54145 biosynthesis but rather plays a role in the hydroxylation of aspartic acid at position 9 during the synthesis of A54145.

### EXAMPLE 2-17

### Constructing φBT1-Based Plasmids for Complementation Experiments

Expression of the modified A54145 biosynthetic pathways in the lptE-I mutant was achieved using an apr resistant BAC-based vector which integrated site-specifically in a neutral site of the S. fradiae genome at the 4C31 attB site. Further complementation of these strains would require the use of a compatible integration plasmid with a different selection marker. The φBT1-based vectors (Gregory et al. J. Bacteriol 2003: 5320-5323) with neomycin (neo) or hygromycin (hyg) resistance markers can integrate site-specifically in a neutral site of the S. fradiae genome at a φBT1 attB site. This can also be achieved in apr resistant strains already containing φC31-based BAC vectors integrated.

A φBT1 integrase-based Streptomyces integrative cassette, removed from MS82 (Gregory et al., 2003, J. Bacteriol: 5320-5323) and contains the 4BT1 integrase and attP site, the oriT from plasmid RK2, and the hyg resistance marker, was engineered by DNA cloning to have flanking DNA regions with identity to the backbone of the BAC vector and the S. fradiae insert. The φBT1 integrative cassette also contains the ermE* constitutive promoter which will drive expression of downstream genes. Homologous recombination between the BAC vector and the Streptomyces integrative cassette flanked by appropriate regions of homology was achieved by transforming the gel purified fragment into an induced E. coli strain carrying the SF1:10 D08 and the Red gene containing plasmid pKD119. These cells were then selected for both hyg and cat resistance and the resulting colonies were analyzed genetically to validate the insertion of the integration cassette and deletion of upstream sequences.

Using the “Red” system the Streptomyces integrative BAC vector pJR2012 was constructed. The φBT1 integrase-based Streptomyces integrative cassette was flanked by DNA regions with identity to the backbone of the BAC vector and lptK of the S. fradiae insert. Homologous recombination between the BAC vector and the Streptomyces integrative cassette was achieved by transforming into cells containing the SF1:10D08 BAC and an induced Red-system (See Example 2-5). The insertion of the φBT1 integrative cassette positions the ermE* constitutive promoter directly in front of lptK to ensure its expression as well as downstream genes remaining on the BAC vector.

Using the “Red” system the Streptomyces integrative BAC vector pJR2015 was constructed. The φBT1 integrase-based Streptomyces integrative cassette was flanked by DNA regions with identity to the backbone of the BAC vector and lptL of the S. fradiae insert. Homologous recombination between the BAC vector and the Streptomyces integrative cassette was achieved by transforming into cells containing the SF1:10D08 BAC and an induced Red-system. The insertion of the 4BT1 integrative cassette positions the ermE* constitutive promoter directly in front of lptL to ensure its expression as well as downstream genes remaining on the BAC vector.

The neo resistant φBT1 pRT802 plasmid was converted to an expression plasmid by the insertion of a cassette containing the ermE* constitutive promoter driving expression of a spectinomycin (spec) resistance marker flanked by the fd and t₀ terminators to create pDA2113. The PCR amplified expression cassette was inserted into the pRT802 plasmid digested with EcoRV and NotI. Complementation plasmids expressing lptI (mGlu12 methyltransferase) or lptL (hAsn3 oxygenase) together were generated by replacing the spec marker and to terminator in pDA2113 with PCR amplified biosynthetic gene and the cat terminator cassette (to terminator engineered in front of the cat resistance gene).

The neo resistant 4BT1 pDA2113 was digested with NdeI and HindIII to remove the spec marker and to terminator and ligated together with the PCR amplified lptI gene, digested with NdeI and XbaI and the cat terminator cassette, digested with XbaI and HindIII. The newly created pDA2129 has the lptI gene, that codes for Glu12 methyltransferase, under the control of the constitutive ermE* promoter.

The neo resistant 4BT1 pDA2113 was digested with NdeI and HindIII to remove the spec marker and to terminator and ligated together with the PCR amplified lptL gene, digested with NdeI and XbaI and the cat terminator cassette, digested with XbaI and HindIII. The newly created pDA2015 has the lptL gene, that codes for Asn3 oxygenase, under the control of the constitutive ermE* promoter.

### EXAMPLE 2-18

### Complementation of an S. Fradiae Mutant Strains Containing φC31 BACS with φBT1-Based Plasmids for the Production of Novel Lipopeptides

Once constructed the plasmid pJ012; containing IptK (Asp9 methoxylase), IptL (Asn3 oxygenase) under the control of the constitutive ermE* promoter and lptI (Glu12 methyltransferase), was introduced into S. fradiae DA1333 by conjugation to create strain DA1449. When fermented and analyzed under the conditions described in Examples 2-13 and 2-14, S. fradiae DA1449 was shown to produce lipopeptides with molecular ions ([M+H]⁺) at m/z of 1644.7, 1644.7, 1658.7, 1658.7, 1658.7, 1672.7, which is in agreement with the masses reported for the major A54145 lipopeptide factors A, A1, B1, B, D, E, respectively, produced by Streptomyces fradiae (Boeck et al., 1990, J. Antibiotics 43: 587-593). This demonstrated that the pJR2012 was able to successfully complement the DA1333 strain to restore lipopeptide production.

The plasmid pJR2012; containing lptK (hAsp9 methoxylase), lptL (Asn3 oxygenase) under the control of the constitutive ermE* promoter and lptI (Glu12 methyltransferase), was introduced into S. fradiae DA1327 by conjugation to create strain DA1553. When fermented and analyzed under the conditions described in Examples 2-13 and 2-14, S. fradiae DA1553 was shown to produce lipopeptides with molecular ions ([M+H]⁺) at m/z of 1628.7, 1628.7 and 1642.7. This is consistent with the masses of analogs of the mGlu12 factors B1, B, and E, respectively, that would have Asp9 instead of moAsp9. DA1553 was shown to produce the Ile13 compounds C114, C115, and C116, although not identified under these fermentation conditions this strain would also have the potential to produce the factors with Val13 C117, C118, and C119. This demonstrated that the putative LptK protein requires the presence of the lptJ protein to hydroxylate Asp9 before methoxylation can occur.

Once constructed the plasmid pJR2015; containing lptL (Asn3 oxygenase) under the control of the constitutive ermE* promoter and lptI (Glu12 methyltransferase), was introduced into S. fradiae DA1336 by conjugation to create strain DA1621. When fermented and analyzed under the conditions described in Examples 2-13 and 2-14, S. fradiae DA1621 was shown to produce lipopeptides with molecular ions ([M+H]⁺) at m/z of 1644.7, 1644.7, 1658.7, 1658.7, 1658.7, 1672.7, which is in agreement with the masses reported for the major A54145 lipopeptide factors A, A1, B1, B, D, E, respectively, produced by Streptomyces fradiae (Boeck et al., 1990, J. Antibiotics 43: 587-593). This demonstrated that the pJR2015 was able to successfully complement the DA1336 strain to restore lipopeptide production.

The plasmid pJR2015; containing IptL (Asn3 oxygenase) under the control of the constitutive ermE* promoter and Iptl (Glu12 methyltransferase), was introduced into S. fradiae DA1333 by conjugation to create strain DA1627. When fermented and analyzed under the conditions described in Examples 2-13 and 2-14, S. fradiae DA1627 was shown to produce lipopeptides with molecular ions ([M+H]⁺) at m/z of 1644.7, 1644.7 and 1658.7. This is consistent with the masses of analogs of the mGlu12 factors B1, B, and E, respectively, that would have hAsp9 instead of moAsp9. DA1627 was shown to produce the Ile13 compounds C111, C112, and C113, although not identified under these fermentation conditions this strain would also have the potential to produce the factors with Val13 C126, C127, and C128.

Once constructed the plasmid pDA2129; containing lptI (Glu12 methyltransferase) under the control of the constitutive ermE* promoter was introduced into S. fradiae DA613 by conjugation to create strain DA1491. When fermented and analyzed under the conditions described in Examples 2-13 and 2-14, S. fradiae DA1491 was shown to produce lipopeptides with molecular ions ([M+H]⁺) at m/z of 1644.7, 1644.7, 1658.7, 1658.7, 1658.7, 1672.7, which is in agreement with the masses reported for the major A54145 lipopeptide factors A, A1, B1, B, D, E, respectively, produced by Streptomyces fradiae (Boeck et al., 1990, J. Antibiotics 43: 587-593). This demonstrated that the pDA2129 was able to successfully complement the DA613 strain to restore production of Glu12 and mGlu12 lipopeptides.

The plasmid pDA2129; containing lptI (Glu12 methyltransferase) under the control of the constitutive ermE* promoter was introduced into S. fradiae DA1327 by conjugation to create strain DA1489. When fermented and analyzed under the conditions described in Examples 2-13 and 2-14, S. fradiae DA1489 was shown to produce lipopeptides with molecular ions ([M+H]⁺) at m/z of 1612.7, 1612.7 and 1626.7. This is consistent with the masses of analogs of the mGlu12 factors B1, B, and E, respectively, that would have Asp9 instead of moAsp9 and Asn3 instead of hAsn3. DA1489 was shown to produce the Ile13 compounds C108, C109, and C110, although not identified under these fermentation conditions this strain would also have the potential to produce the factors with Val13 C138, C139, and C140.

The plasmid pDA2129; containing lptI (Glu12 methyltransferase) under the control of the constitutive ermE* promoter was introduced into S. fradiae DA1327 by conjugation to create strain DA1489. Into this strain was added the plasmid pDA2076 to create DA2000 When fermented and analyzed under the conditions described in Examples 2-13 and 2-14, S. fradiae DA2000 was shown to produce lipopeptides with molecular ions ([M+H]⁺) at m/z of 1642.7, 1642.7 and 1656.7. This is consistent with the masses of analogs of the mGlu12 factors B1, B, and E, respectively, that would have Asn3 instead of hAsn3. DA1489 was shown to produce the Ile13 compounds C96, C97, and C98, although not identified under these fermentation conditions this strain would also have the potential to produce the factors with Val 13 C141, C142, and C143.

The plasmid pDA2129; containing lptI (Glu12 methyltransferase) under the control of the constitutive ermE* promoter was introduced into S. fradiae DA1333 by conjugation to create strain DA1459. When fermented and analyzed under the conditions described in Examples 2-13 and 2-14, S. fradiae DA1459 was shown to produce lipopeptides with molecular ions ([M+H]⁺) at m/z of 1628.7, 1628.7 and 1642.7. This is consistent with the masses of analogs of the mGlu12 factors B1, B, and E, respectively, that would have hAsp9 instead of moAsp9 and Asn3 instead of hAsn3. DA1489 was shown to produce the Ile13 compounds C99, C100, and C101, although not identified under these fermentation conditions this strain would also have the potential to produce the factors with Val13 C129, C130, and C131.

Once constructed the plasmid pDA2117; containing lptL (Asn3 oxygenase) under the control of the constitutive ermE* promoter was introduced into S. fradiae DA1336 by conjugation to create strain DA1470. When fermented and analyzed under the conditions described in Examples 2-13 and 2-14, S. fradiae DA1470 was shown to produce lipopeptides with molecular ions ([M+H]⁺) at m/z of 1644.7, 1644.7, and 1658.7 which is in agreement with the masses reported for the major A54145 lipopeptide factors A, A1, D, respectively, produced by Streptomyces fradiae that have Glu12 instead of mGlu12 (Boeck et al., 1990, J. Antibiotics 43: 587-593). This demonstrated that the pDA2117 was able to successfully complement the DA1336 strain to restore production of Glu12 lipopeptides.

The plasmid pDA2117; containing lptL (Asn3 oxygenase) under the control of the constitutive ermE* promoter was introduced into S. fradiae DA1327 by conjugation to create strain DA1484. When fermented and analyzed under the conditions described in Examples 2-13 and 2-14, S. fradiae DA1484 was shown to produce lipopeptides with molecular ions ([M+H]⁺) at m/z of 1614.7, 1614.7 and 1628.7. This is consistent with the masses of analogs of the Glu12 factors A, A1, and D, respectively, that would have Asp9 instead of moAsp9. DA1484 was shown to produce the Ile13 compounds C90, C91, and C92, although not identified under these fermentation conditions this strain would also have the potential to produce the factors with Val13 C120, C121, and C122.

The plasmid pDA2117; containing lptL (Asn3 oxygenase) under the control of the constitutive ermE* promoter was introduced into S. fradiae DA1333 by conjugation to create strain DA1453. When fermented and analyzed under the conditions described in Examples 2-13 and 2-14, S. fradiae DA1453 was shown to produce lipopeptides with molecular ions ([M+H]⁺) at m/z of 1630.7, 1630.7 and 1644.7. This is consistent with the masses of analogs of the Glu12 factors A, A1, and D, respectively, that would have hAsp9 instead of moAsp9. DA1453 was shown to produce the Ile13 compounds C87, C88, and C89, although not identified under these fermentation conditions this strain would also have the potential to produce the factors with Val13 C123, C124, and C125.

### EXAMPLE 2-20

### Module Exchanges Constructed at Positions 8 or 11 in A54145

Module exchanges to change either position 8 or 11 of A54145 core was done on plasmid pDA2054 (see Example 2-16, plasmid that is capable of expressing lptABCD—without PmeI site). This plasmid was able to restore A54145 biosynthesis in ΔlptE-I S. fradiae. The DNA fragment coding for module 8 or module 11 on pDA2054 was replaced by genR gene at the linker regions B and CAT, using the red-mediated recombination system described in Example 2-6 (see primers used below). Once the genR gene was inserted into pDA2054 replacing either the lysine CAT module at position 8 or the asparagines module at position 11 was removed by NheI/PmeI digest (module 8) or AvrII/PmeI (module 11). The modules that were used to replace either lysine or asparagines were cloned using the gap-repair technique described in Example 2-6 and could be removed from pBR322 using the restriction sites NheI/HpaI. These modules included DNA fragment coding for heterologous modules cloned from daptomycin dpt D-Ala 8, dpt D-Ser 11 or from A54145 lpt D-Asn11. All possible combinations of Ser, Ala and Asn at either positions 8 or 11 were constructed and designated as the following hybrid plasmids pKN56 (pDA2054 containing D-Ala8), pKN57 (pDA2054 containing D-Ser8), pKN58 (pDA2054 containing -D-Asn8), pKN59 (pDA2054 containing -D-Ala11) and pKN60 (pDA2054 containing -D-Ser11) were introduced into S. fradiae mutants DA1187 (ΔlptE-1, see Example 2-12) and DA740 (DA1187 plus plasmid pDA2129 which expresses lptI, see Example 2-18) to generate hybrid lipopeptides with 2 changes at positions 8 or 11 and 12.

Primers for deletion
lpt-D-Lys8
lpt-Del-Lys8-B-Nhe

(SEQ ID NO: 29)

5′-GTG TTC GAG GCC CGA ACG GTC GCC GCG CTG GCG GCC
CGG CTG CGG ACC GCGCT AGC TGTG TAG GCT GGA GCT GCT
TCG-3′
lpt-Lys8-CAT-II:

(SEQ ID NO 30)

5′-CGG CGA GAG CGG GGT CCT CGT CGC CTG CCG CGT CGG
TCC TGC GGG GTTTAAACCATATGAATATCCTCCTTA-3′
lpt-D-Asn11
lpt-Asn11-B.

(SEQ ID NO: 31)

5′-CGAGACACCGACCGTGGCCGGTCTCGCCGCCGCGCTCTCCGCGGCCC
TAGGTGTGTAGGCTGGAGCTGCTTCG-3′
lpt-Asn11-CAT.

(SEQ ID NO: 32)

5′-GTCCCGCGACCGCCGAGTACCTCGGTCGCCGGACCGCCGGGGCGCGG
TTTAAACCATATGAATATCCTCCTTA-3′
Primers for gap-repair cloning of both dpt-Ala8
and dpt-Ser11 modules
Ala/Ser-B-P13

(SEQ ID NO: 33)

5′-CGTCCGCTCCCGTGCCACCAGAGGCACCCGCACCCCCAAAGCCGAC
GCTAGCTTCTTAGACGTCAGGTGGCAC-3′
Ser-CAT-P14-II

(SEQ ID NO: 34)

5′-GTTCGGGATGTTTTCGAGGGCCGTACGGTACGTGCTCTGGCGGCTGT
GGTTAACCGATACGCGAGCGAACGTGA-3′

Strain KN707 was constructed by adding pKN56 to DA1187. When fermented and analyzed under the conditions described in Examples 2-13 and 2-14, this strain was shown to produce compounds C313, C314, C315, C316, C317 and C318.

Strain KN681 was constructed by adding pKN56 to DA740. When fermented and analyzed under the conditions described in Examples 2-13 and 2-14, this strain was shown to produce compounds C319, C320, C321, C322, C323 and C324.

Strain KN715 was constructed by adding pKN57 to DA1187. When fermented and analyzed under the conditions described in Examples 2-13 and 2-14, this strain was shown to produce compounds C292, C293, C294, C295, C296 and C297.

Strain KN689 was constructed by adding pKN57 to DA740. When fermented and analyzed under the conditions described in Examples 2-13 and 2-14, this strain was shown to produce compounds C289, C290, C291, C298, C299 and C300.

Strain KN723 was constructed by adding pKN58 to DA1187. When fermented and analyzed under the conditions described in Examples 2-13 and 2-14, this strain was shown to produce compounds C3 07, C308, C309, C310, C311 and C312.

Strain KN697 was constructed by adding pKN58 to DA740. When fermented and analyzed under the conditions described in Examples 2-13 and 2-14, this strain was shown to produce compounds C301, C302, C303, C304, C305 and C306.

Strain KN728 was constructed by adding pKN59 to DA1187. When fermented and analyzed under the conditions described in Examples 2-13 and 2-14, this strain was shown to produce compounds C334, C335, C336, C337, C338 and C339.

Strain KN701 was constructed by adding pKN59 to DA740. When fermented and analyzed under the conditions described in Examples 2-13 and 2-14, this strain was shown to produce compounds C328, C329, C330, C331, C332 and C333.

Strain KN730 was constructed by adding pKN60 to DA1187. When fermented and analyzed under the conditions described in Examples 2-13 and 2-14, this strain was shown to produce compounds C147, C148, C149, C325, C326 and C327.

Strain KN705 was constructed by adding pKN60 to DA740. When fermented and analyzed under the conditions described in Examples 2-13 and 2-14, this strain was shown to produce compounds C150, C151, C152, C153, C154 and C155.

### EXAMPLE 2-21

### Module Exchanges Constructed at Positions 2 Through 4 in A54145

The exchange of modules 2-4 in lptA (D-Glu-2/hAsn-3/Thr-4) was constructed on pDA2054 (this plasmid expresses lptABCD from a BAC vector, see Example 2-16 for its construction). pDA2054 was able to restore biosynthesis of the glutamate derivative of A54145 in the mutant DA1187 (see Example 2-15). The DNA fragment coding for modules 2-4 in lptA, on pDA2054 was replaced by the genR gene between the linker regions B and CAT (module exchange 2-4) using the red-mediated recombination system described in Example 2-6. The genR gene was flanked by restriction sites for NheI/PmeI which allowed its easy removal from the BAC vector through restriction digest. The replacement fragment was cloned into pBR322 from dptA using the gap-repair system described in Example 2-6 and was flanked by restriction sites for NheI/HpaI. This fragment from dptA encoded for modules 2-4 from daptomycin (D-Asn-2/Asp-3/Thr-4), this fragment was ligated to the 2-4 deleted version of pDA2054 to generate pKN61. Plasmid pKN61 was introduced into DA1187 and XH1000 (DA1189 carrying pKN55, see Example 2-10) to produce recombinant strains KN650 (DA1187 plus pKN651) and KN665 (XH1000 plus pKN61).

When fermented and analyzed under the conditions described in Examples 2-13 and 2-14, strain KN650 was shown to produce compounds C271, C272, C273, C274, C275 and C276.

When fermented and analyzed under the conditions described in Examples 2-13 and, 2-14, strain KN665 was shown to produce compounds C265, C266, C267, C268, C269 and C270.

Primers for deletion
Lpt2-4
lpt-Del-Glu2-B-Nhe:

(SEQ ID NO: 35)

CCG GTC CCC GAC CGT CGC CCG CCT CGC GGA GGA ACT
GGG CGA CGG GCTAGCTGTGTAGGCTGGAGCTGCTTCG
lptGlu2-CAT:

(SEQ ID NO: 36)

5′-CCT GCG GCG CGG GAC GCT CCG CGT CCG CGT CCG GTC
CGG CGG ACCGTTTAAACCATATGAATATCCTCCTTA-3′
Primers for gap repair cloning
Dpt2-4
dpt-Asn2-Pick-B:

(SEQ ID NO: 37)

AGGCGCTCCGGGCGCGGAGGCAGCGGCGGGGTGGTGTGCTGCCGTCCG
GCTAGCTTCTTAGACGTCAGGTGGCAC
dpt-Thr4-Pick-CAT:

(SEQ ID NO: 38)

CTCTTCGCCGCGCCCACGCCTGCCGGGCTCGCGACCGTACTGGCGGCC
GTTAACCGATACGCGAGCGAACGTGA

### EXAMPLE 2-22

### Module Exchanges Constructed at Positions 2 Through 8 in A54145

The exchange of modules 2-8 in lptA,B,C (D-Glu-2/hAsn-3/Thr-4/Sar-5/Ala-6/Asp-7/D-Lys-8) was constructed on pDA2054 (this plasmid expresses lptABCD from a BAC vector, see Example 2-16 for its construction). pDA2054 was able to restore biosynthesis of the glutamate derivative of A54145 in the mutant DA1187 (see Example 2-15). The DNA fragment coding for modules 2-8 in lptA,B,C, on pDA2054 was replaced by the genR gene between the linker regions B and CATE (module exchange 2-8) using the red-mediated recombination system described in Example 2-6. The genR gene was flanked by restriction sites for NheI/PmeI which allowed its easy removal from the BAC vector through restriction digest. The replacement fragment was cloned into pBR322 from dptA,BC using the gap-repair system described in Example 2-6 and was flanked by restriction sites for NheI/HpaI. This fragment from dptA,BC encoded for modules 2-8 from daptomycin (D-Asn-2/Asp-3/Thr-4/Gly-5/Orn-6/Asp-7/D-Ala-8), this fragment was ligated to the 2-8 deleted version of pDA2054 to generate pKN62. Plasmid pKN62 was introduced into DA1187 and XH1000 (DA1189 carrying pKN55, see Example 2-10) to produce recombinant strains KN653 (DA1187 plus pKN62) and KN669 (XH1000 plus pKN62).

When fermented and analyzed under the conditions described in Examples 2-13 and 2-14, strain KN653 was shown to produce compounds C283, C284, C285, C286, C287 and C288.

When fermented and analyzed under the conditions described in Examples 2-13 and 2-14, strain KN669 was shown to produce compounds C277, C278, C279, C280, C281 and C282.

Primers for deletion
Lpt2-8
lpt-Del-Glu2-B-Nhe:

(SEQ ID NO: 39)

CCG GTC CCC GAC CGT CGC CCG CCT CGC GGA GGA ACT
GGG CGA CGG GCTAGCTGTGTAGGCTGGAGCTGCTTCG
lpt-Lys8-CATE2-II:

(SEQ ID NO: 40)

5′GGGG GGC GAC CGG CAG GAT GTC CTC CAA GGC GGT GCC
GGT GCG GC GTTTAAACCATATGAATATCCTCCTTA 3′
Primers for gap repair cloning
Dpt2-8
dpt-Asn2-Pick-B:

(SEQ ID NO: 41)

AGGCGCTCCGGGCGCGGAGGCAGCGGCGGGGTGGTGTGCTGCCGTCCG
GCTAGCTTCTTAGACGTCAGGTGGCAC
Ala-CATE2-P14-II

(SEQ ID NO: 42)

CGACGTGACGCTGGTGGAAGTGAACCAGGTGGAGCTCGACCGTCTGCAGG
TTAACCGATACGCGAGCGAACGTGA

### EXAMPLE 2-23

### Deletion of Methylation in Sarcosine Module to Produce Glycine at Position 5 in A54145

The selection cassette for the deletion of the methylation domain within lptA5-Sar module was amplified with PCR primers that carry 50 bp of homology to the linker region of the domain under investigation (see FIG. 5 for positions of linkers). When these PCR fragments were introduced into electro-competent cells that contained pDA2054 (a truncated version of the lptBAC that contains the entire lpt pathway, see Example 2-16) and induced Red-system (see Example 2-6), the resistance cassette was integrated site specifically at the target site in pDA2054 by homologous recombination (FIG. 3).

These cells were then selected for the presence of the gent resistance marker, and the resulting colonies were analyzed genetically to validate the construction of the appropriate deletion or disruption. Part of the primer design involves placing a restriction site within the linker region of interest (PmeI and SwaI). (FIG. 3).

The BAC containing the gent deletion of the methylation domain was subsequently digested using the unique restriction sites PmeI and SwaI to excise the selection marker and religated, and the resulting colonies were analyzed genetically to validate the construction of the appropriate deletion of the methylation domain. The resulting clone was named pSD409.

pSD409 was added by interspecies conjugation from E. coli to DA1187 (a lptE-I deletion of S. fradiae, see Example 2-12) to create SD409. S. fradiae SD409 was fermented and analyzed using the techniques described in Examples 2-13 and 2-14 respectively.

The identification of lipopeptides was confirmed, as indicated by molecular ions ([M+H]⁺) at m/z of 1616.7 (C183), 1630.7 (C182) 1630.7 (C181) and 1644.7 (C180), which is in agreement with the masses reported for the major A54145 lipopeptides Streptomyces fradiae—14 m/z. Although not identified under these fermentation conditions this strain would also have the potential to produce the factors with Val13 C184, and C185.

### EXAMPLE 2-24. MODULE EXCHANGES CONSTRUCTED AT POSITION 2 IN A54145

Module exchanges were done on plasmid plpt-J14-P to replaced D-Glu-2 module by the module for D-Asn, D-Ser or D-Ala. The plasmid pDA2054 was able to restore biosynthesis of the glutamate derivative of A54145 in the mutant DA1187. The DNA fragment coding for module 2 on pDA2054 was first replaced by genR gene at the linker regions CAT (see Example 2-20). The genR gene was removed by NheI/PmeI digest and replaced by NheI/HpaI DNA fragment coding for lpt D-Asn11, (cloned by gap-repair method as described in Example 2-5 using the primers Lpt-N11-B-P13 and Lpt-N11-CAT-P14). This created the hybrid plasmid pXH2000 which was introduced into DA1189 and XU1000 (DA1189 carrying pKN55) to produce recombinant strains XH1001(DA1889 plus pXH2000) and XH1002 (XH1000 plus pXH2000). XH1001 and XH1002 were fermented and analyzed using the protocols described in Example 2-13 and 2-14. S. fradiae XH1001 was shown to produce lipopeptides with molecular ions ([M+H]⁺) at m/z of 1629.7, 1629.7 and 1643.7. This is consistent with the masses of analogs of the Glu12 factors A, A1, and D, respectively, that would have Asn-2. XH1001 was shown to produce the Ile13 compounds C343, C344, and C345. Although not identified under these fermentation conditions this strain would also have the potential to produce the factors with Val13 C346, C347, and C348. S. fradiae XH1002 was shown to produce lipopeptides with molecular ions ([M+H]⁺) at m/z of 1643.7, 1643.7 and 1657.7. This is consistent with the masses of analogs of the mGlu12 factors B, B1, and E, respectively, that would have Asn-2. XH1002 was shown to produce the Ile13 compounds C340, C341, and C342. Although not identified under these fermentation conditions this strain would also have the potential to produce the factors with Val13 C349, C350, and C351.

Primers for deletion of D-Glu-2 module
lptGlu2-B:

(SEQ ID NO: 43)

5′-CCG GTC CCC GAC CGT CGC CCG CCT CGC GGA GGA ACT
GGG CGA CGG CCTAGGTGTGTAGGCTGGAGCTGCTTCG-3′
lptGlu2-CAT:

(SEQ ID NO: 44)

5′-CCT GCG GCG CGG GAC GCT CCG CGT CCG CGT CCG GTC
CGG CGG ACCGTTTAAACCATATGAATATCCTCCTTA-3′
Primers for gap-repair of lpt-Asn-11.
Lpt-N11-B-P13

(SEQ ID NO: 45)

TCGGGGCGCGGGTCGGCGGGGCGCAGCCGGGGTCCGGCCTCGCCC
GCTAGCTTCTTAGACGTCAGGTGGCAC
Lpt-N11-CAT-P14

(SEQ ID NO: 46)

CGCGACATCTTCGAACAGCGCACGCCCGCCGCCCTCGCCGGCCGC
GTTAACCGATACGCGAGCGAACGTGA

### EXAMPLE 3-1

### Biological Activity

Compounds according to Formula I were tested for antimicrobial activity against a panel of organisms according to standard procedures described by the National Committee for Clinical Laboratory Standards (NCCLS document M7-A6, Vol. 23, Number 2, 2003) except that all testing was performed at 37° C. and under constant agitation at 200 rpm. Compounds were dissolved in either 100% dimethyl sulfoxide or water or 50:50 mix by volume of dimethyl sulfoxide and water depending upon the solubility of the compound and were diluted to the final reaction concentration (0.11 μg/mL-100 μg/mL) in microbial growth media. In all cases the final concentration of dimethyl sulfoxide incubated with cells is less than or equal to 1%. For minimum inhibitory concentration (MIC) calculations, 2-fold dilutions of compounds were added to wells of a microtiter plate containing 5×10⁴ bacteria cells in a final volume of 100 μL of media (Mueller-Hinton Broth supplemented with 50 mg/L Ca²⁺). The optical densities (OD) of the bacterial cells, which measures bacterial cell growth and proliferation, were measured using a commercial plate reader. The MIC value is defined as the lowest compound concentration inhibiting growth of the test organism. The MIC (in μg/ml) value of representative compounds of the present invention are listed in Table V.

TABLE V
Biological Activity of Compounds of Formula I

| | Assay Strain ^{###} | | | | | | | |
|---|---|---|---|---|---|---|---|---|
| # | SAU.42 | SAU.399 | SAU.278 | EFM.14 | EFM.384 | EFS.201 | EFS.312 | SPN.402 |
| C1 | ++ | ++ | + | ++ | + | ++ | + | ++ |
| C2 | +++ | +++ | + | ++ | + | ++ | + | +++ |
| C3 | +++ | +++ | ++ | ++ | ++ | +++ | + | +++ |
| C4 | ++ | ++ | + | + | + | + | + | ++ |
| C5 | ++ | ++ | + | ++ | + | ++ | + | ++ |
| C6 | +++ | +++ | + | ++ | + | ++ | + | ++ |
| C7 | ++ | ++ | + | + | + | + | + | ++ |
| C8 | ++ | ++ | + | ++ | + | + | + | ++ |
| C9 | +++ | ++ | + | ++ | + | ++ | + | ++ |
| C10 | ++ | ++ | + | ++ | + | ++ | + | ++ |
| C11 | ++ | ++ | + | ++ | + | ++ | + | ++ |
| C12 | ++ | ++ | + | ++ | ++ | ++ | + | |
| C16 | + | | | | | | | |
| C17 | + | | | | | | | |
| C18 | ++ | | | | | | | |
| C21 | + | | | | | | | |
| C22 | +++ | | | | | | | |
| C23 | +++ | | | | | | | |
| C24 | +++ | | | | | | | |
| C25 | ++ | | | | | | | |
| C26 | +++ | | | | | | | |
| C27 | +++ | | | | | | | |
| C37 | ++ | | | | | | | |
| C38 | ++ | | | | | | | |
| C39 | ++ | | | | | | | |
| C46 | +++ | +++ | ++ | +++ | ++ | +++ | + | |
| C47 | +++ | +++ | ++ | +++ | ++ | +++ | ++ | |
| C48 | +++ | +++ | +++ | +++ | ++ | +++ | ++ | |
| C49 | +++ | | | | | | | |
| C61 | ++ | | | | | | | |
| C62 | ++ | | | | | | | |
| C70 | + | | | | | | | |
| C71 | + | | | | | | | |
| C72 | + | | | | | | | |
| C73 | + | | | | | | | |
| C74 | + | | | | | | | |
| C75 | + | | | | | | | |
| C76 | + | | | | | | | |
| C77 | + | | | | | | | |
| C78 | + | | | | | | | |
| C79 | + | | | | | | | |
| C80 | + | | | | | | | |
| C81 | + | | | | | | | |
| C82 | + | | | | | | | |
| C83 | + | | | | | | | |
| C84 | + | | | | | | | |
| C85 | + | | | | | | | |
| C86 | + | | | | | | | |
| C87 | + | | | | | | | |
| C90 | + | | | | | | | |
| C93 | + | | | | | | | |
| C94 | + | | | | | | | |
| C95 | + | | | | | | | |
| C96 | ++ | | | | | | | |
| C97 | ++ | | | | | | | |
| C98 | ++ | | | | | | | |
| C103 | + | | | | | | | |
| C105 | + | | | | | | | |
| C108 | ++ | | | | | | | |
| C146 | + | | | | | | | |
| C153 | ++ | | | | | | | |
| C154 | + | | | | | | | |
| C155 | ++ | | | | | | | |
| C180 | + | | | | | | | |
| C189 | ++ | | | | | | | |
| C190 | ++ | | | | | | | |
| C191 | +++ | | | | | | | |
| C201 | + | | | | | | | |
| C202 | ++ | | | | | | | |
| C203 | ++ | | | | | | | |
| C204 | + | | | | | | | |
| C205 | + | | | | | | | |
| C206 | + | | | | | | | |
| C210 | + | | | | | | | |
| C211 | ++ | | | | | | | |
| C212 | ++ | | | | | | | |
| C233 | ++ | | | | | | | |
| C234 | ++ | | | | | | | |
| C235 | ++ | | | | | | | |
| C236 | ++ | | | | | | | |
| C237 | +++ | | | | | | | |
| C238 | ++ | | | | | | | |
| C325 | + | | | | | | | |
| C326 | + | | | | | | | |
| C327 | + | | | | | | | |
| C352 | + | | | | | | | |
| C353 | + | | | | | | | |
| C354 | + | | | | | | | |
| C355 | + | | | | | | | |
| C356 | + | | | | | | | |
| C357 | + | | | | | | | |
| C358 | + | | | | | | | |
| C359 | + | | | | | | | |
| C360 | + | | | | | | | |
| C361 | + | | | | | | | |
| C362 | + | | | | | | | |
| C363 | + | | | | | | | |
| C364 | + | | | | | | | |
| C365 | + | | | | | | | |
| C366 | + | | | | | | | |
| C367 | + | | | | | | | |
| C368 | + | | | | | | | |
| C369 | + | | | | | | | |

wherein:

| Strain # | Species | ATCC# | Strain description |
|---|---|---|---|
| SAU.42 | Staphylococcus | 29213 | NCCLS reference strain for broth microdilution MIC |
| | aureus | | assay obtained from the ATCC |
| SAU.399 | Staphylococcus | 43300 | NCCLS Methicillin and Oxacillin Resistant Clinical |
| | aureus | | Isolate obtained from the ATCC |
| SAU.278 | Staphylococcus | n/a | Daptomycin resistant mutant (D10)- liquid serial |
| | aureus | | passage mutant derived from parent S. aureus 42 |
| EFM.14 | Enterococcus | 6569 | FDA test organism in AOAC test for germicidal activity |
| | faecium | | obtained from the ATCC |
| EFM.384 | Enterococcus | n/a | Daptomycin resistant mutant (14-A)-liquid serial |
| | faecium | | passage mutant derived from parent E. faecium 14 |
| EFS.201 | Enterococcus | 49452 | Quality control strain for API products obtained from |
| | faecalis | | the ATCC |
| EFS.312 | Enterococcus | n/a | Daptomycin resistant mutant (EFA)- liquid serial |
| | faecalis | | passage mutant derived from parent E. faecalis 201 |
| SPN.402 | Streptococcus | 6303 | Public health Report 59: 449-468 (serotype 3) obtained |
| | pnuemoniae | | from the ATCC |

Wherein “+++” indicates that the compound has an MIC (μg/ml) of 1 μg/ml or less or an ED₅₀ of 1 mg/kg or less;

“++” indicates that the compound has an MIC (μg/ml) or ED₅₀ of greater than 1 μg/ml or 1 mg/kg, respectively but less than or equal to 10 μg/ml or ED₅₀ of 10 mg/kg, respectively; and

“+” indicates that the compound has an MIC (μg/ml) of greater than 10 μg/ml or an ED₅₀ of greater than 10 mg/kg.

### EXAMPLE 3-2

### In Vivo Activity

The mouse protection test is an industry standard for measuring the efficacy of a test compound in vivo (for examples of this model see Clement, J J. et al., 1994, Antimicrobial Agents and Chemotherapy 38 (5): 1071-1078). As exemplified below, this test is used to demonstrate the in vivo efficacy of the compounds of the present invention against bacteria.

The in vivo antibacterial activity is established by infecting female CD-I mice (Charles River Lab, MA) weighing 19-23 g intraperitoneally with Methicillin Resistant S. aureus (MRSA) inoculum. The inoculum is prepared from Methicillin Resistant S. aureus (ATCC 43300). The MRSA inoculum is cultured in Mueller-Hinton (MH) broth at 37° C. for 18 hours. The optical density at 600 nm (OD₆₀₀) is determined for a 1:10 dilution of the overnight culture. Bacteria (8×10⁸ cfu) is added to 20 ml of phosphate buffered saline (Sigma P-0261) containing 5% hog gastric mucin (Sigma M-2378). All animals are injected with 0.5 ml of the inoculum, equivalent to 2×10⁷ cfu/mouse, which is the dose causing ˜100% death of the animals without treatment.

The test compound is dissolved in 10.0 ml of saline solution to give a solution of 1 mg/ml (pH=7.0). This solution is serially diluted with vehicle by 4-fold (1.5 ml to 6.0 ml) to give 0.25, 0.063 and 0.016 mg/ml solutions. All the solutions are filtered with 0.2 μm Nalgene syringe filter. One hour after the bacterial inoculation, group 1 animals are subcutaneously (sc) injected with buffer (no test compound) and groups 2 to 5 were given test compound sc at 10.0, 2.5, 0.63, and 0.16 mg/kg, respectively. Group 6 animals receive test compound sc at 10 mg/kg (or the highest therapeutic dose of a given compound) only for monitoring acute toxicity. These injections are repeated once at 4 hours after the inoculation for the respective groups. The injection volume at each time is 10 ml per kilogram of body weight. The 50% protective dose (PD₅₀) is calculated on the basis of the number of mice surviving 7 days after inoculation.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings herein that certain changes and modifications may be made thereto without departing from the spirit or scope of the invention disclosed.

1-59. (canceled)

60. A composition comprising a compound of Formula F2: 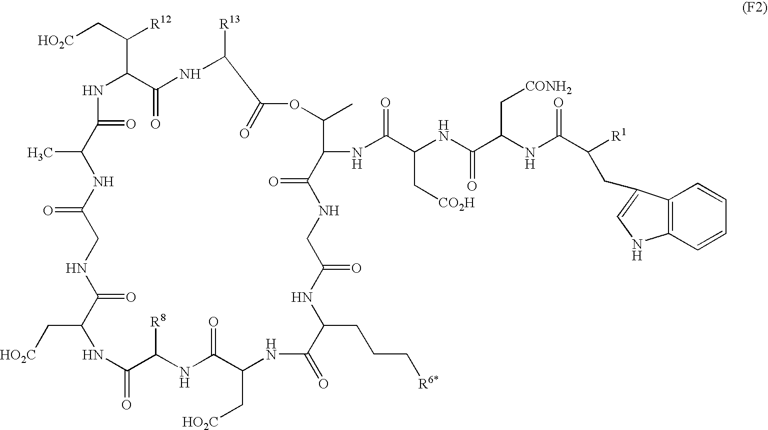
and salts thereof; wherein:
a) R⁸ is hydrogen, methyl, 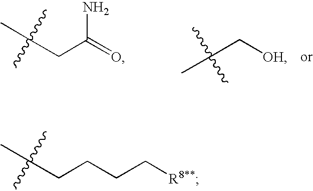
b) R¹² is H or CH₃;
c) R¹³ is CH(CH₃)₂, CH(CH₂CH₃)CH₃, 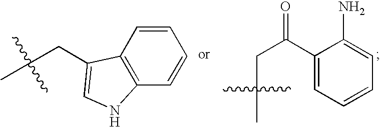
and
d) each of R¹, R⁶* and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

61. The compound of claim 60 wherein each of R⁶* and R⁸** is independently amino, NH-amino protecting group, or carbamoyl.

62. The compound of claim 61 wherein each of R⁶* and R⁸** is independently amino.

63. The compound of claim 60 wherein R¹ is amino, alkanoylamino, NH-amino protecting group.

64. The compound of claim 63 wherein R¹ is a C₁₀-C₁₃ alkanoylamino.

65. The compound of claim 64 wherein R¹ is

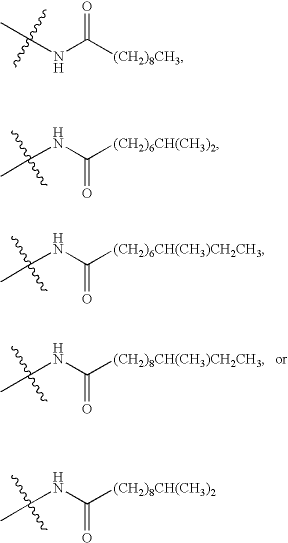

66. The compound of claim 60 selected from

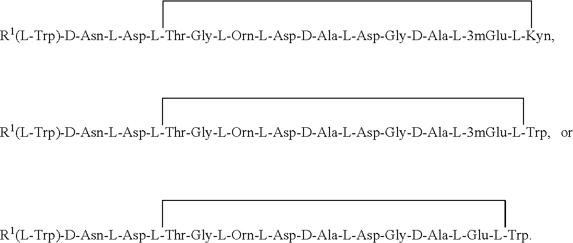

67. The compound of claim 60 selected from:

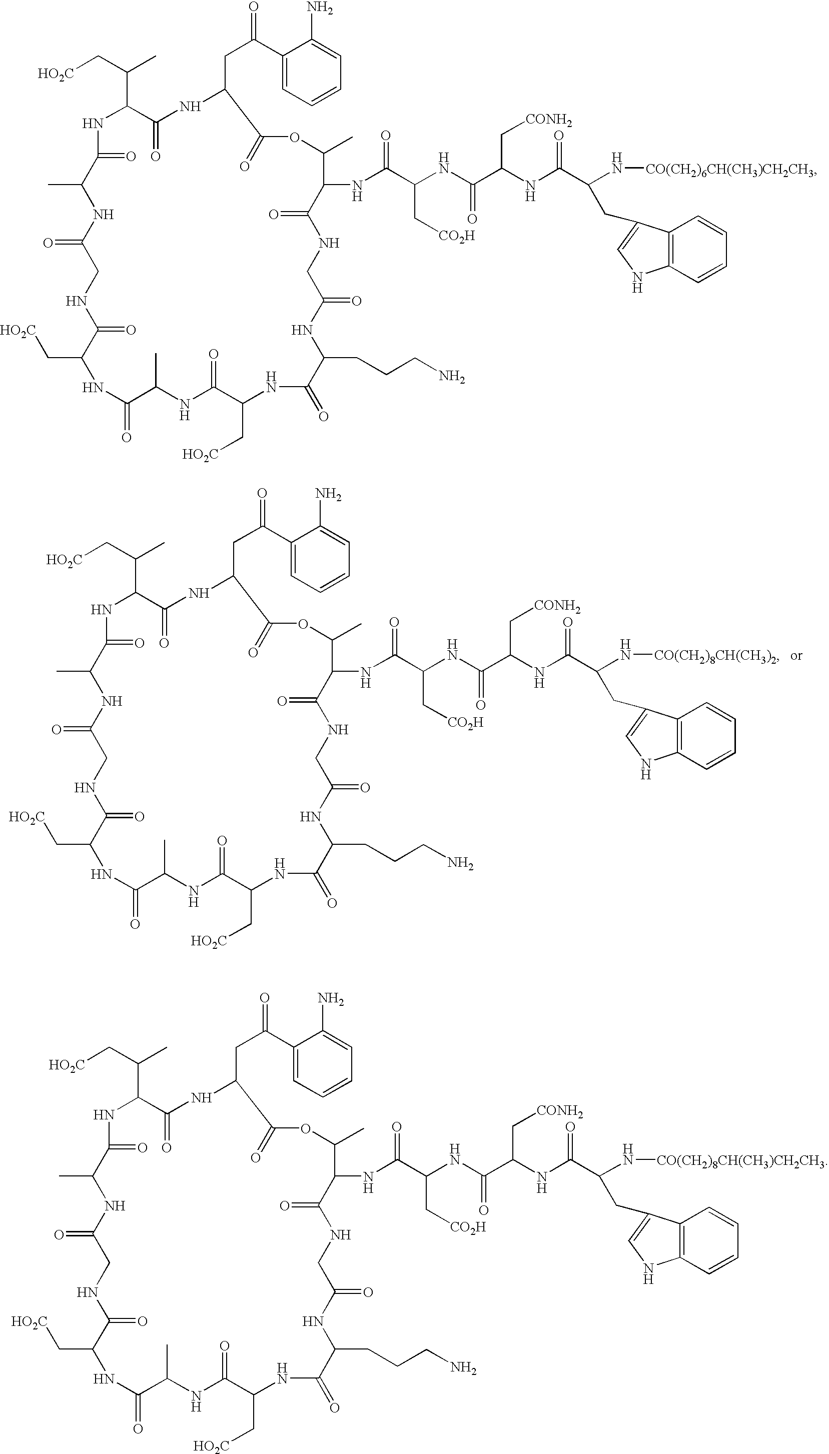

68. A composition comprising a compound of Formula F9: 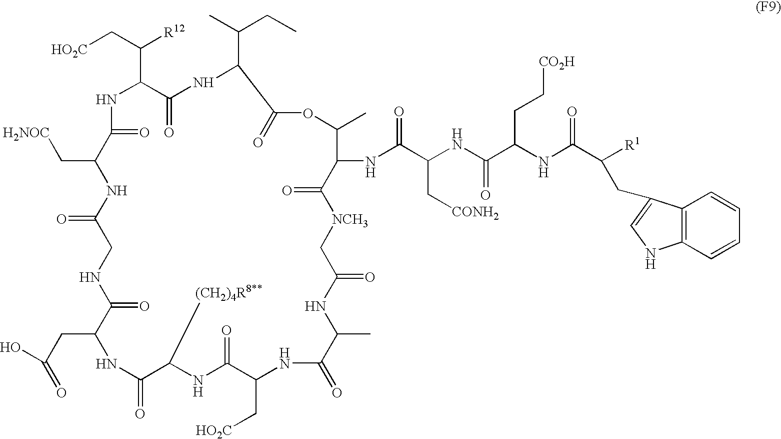
and salts thereof; wherein:
a) R¹² is H or CH₃; and
b) each of R¹, and R⁸** is independently amino, monosubstituted amino, disubstituted amino, NH-amino protecting group, acylamino, ureido, guanidino, carbamoyl, sulfonamino, thioacylamino, thioureido, iminoamino, or phosphonamino.

69. The compound of claim 68 wherein R⁸** is amino, NH-amino protecting group, or carbamoyl.

70. The compound of claim 69 wherein R⁸** is amino.

71. The compound of claim 68 wherein R¹ is amino, alkanoylamino, NH-amino protecting group.

72. The compound of claim 71 wherein R¹ is a C₁₀-C₁₃ alkanoylamino.

73. The compound of claim 72 wherein R¹ is

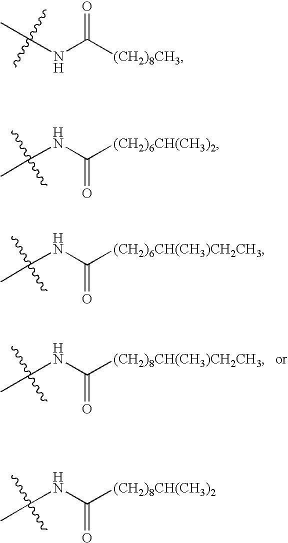

74. The compound of claim 68 wherein R¹² is CH₃.

75. The compound of claim 74 wherein R¹ is alkanoylamino.

76. The compound of claim 75 wherein R¹ is C₁₁-alkanoylamino.

77. The compound of claim 76 wherein R¹ is

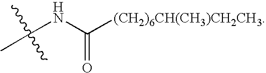

78. The compound of claim 68 selected from:

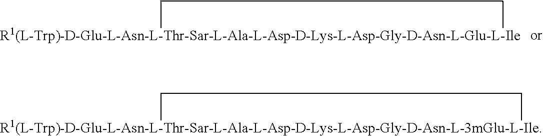

79. The compound of claim 68 selected from

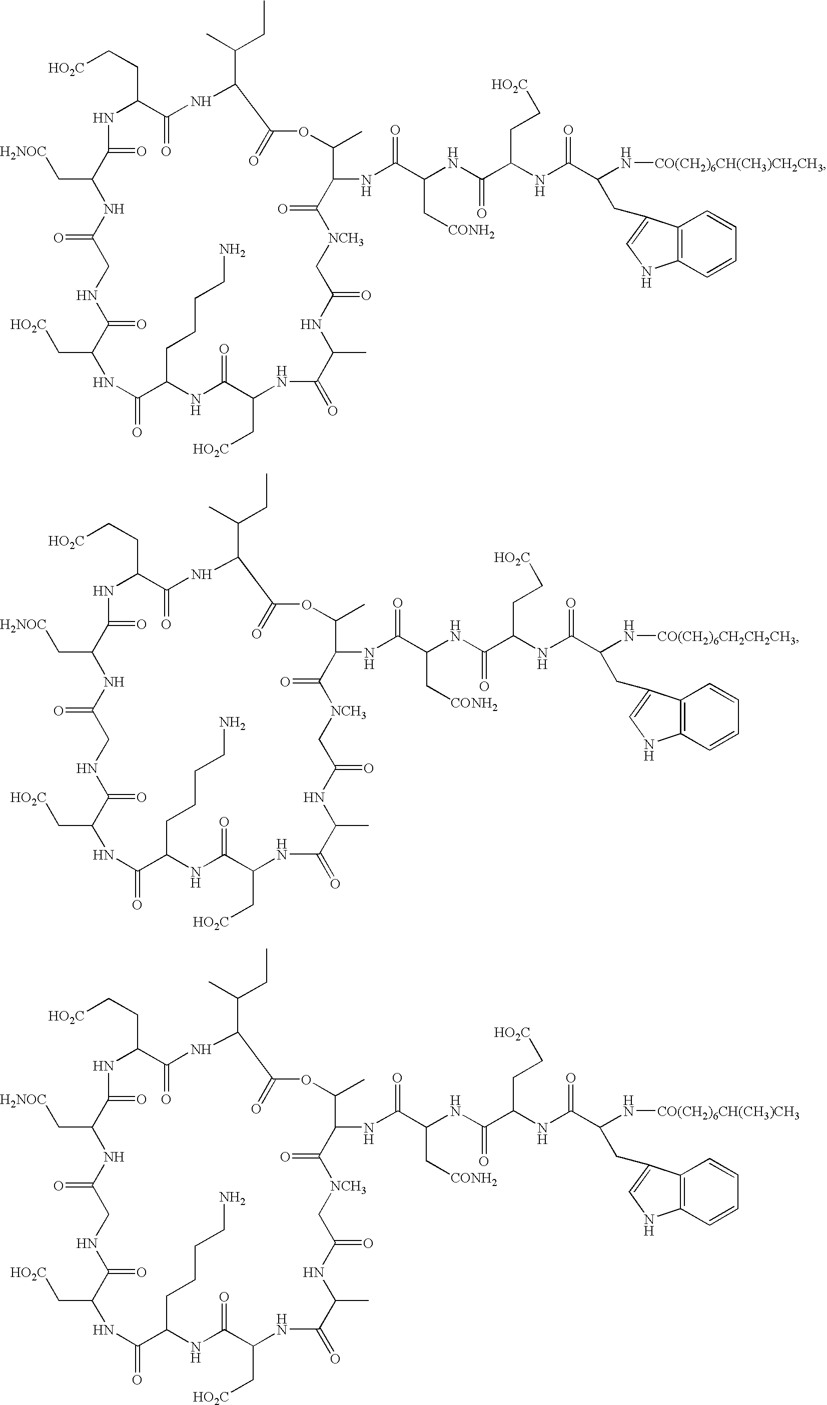

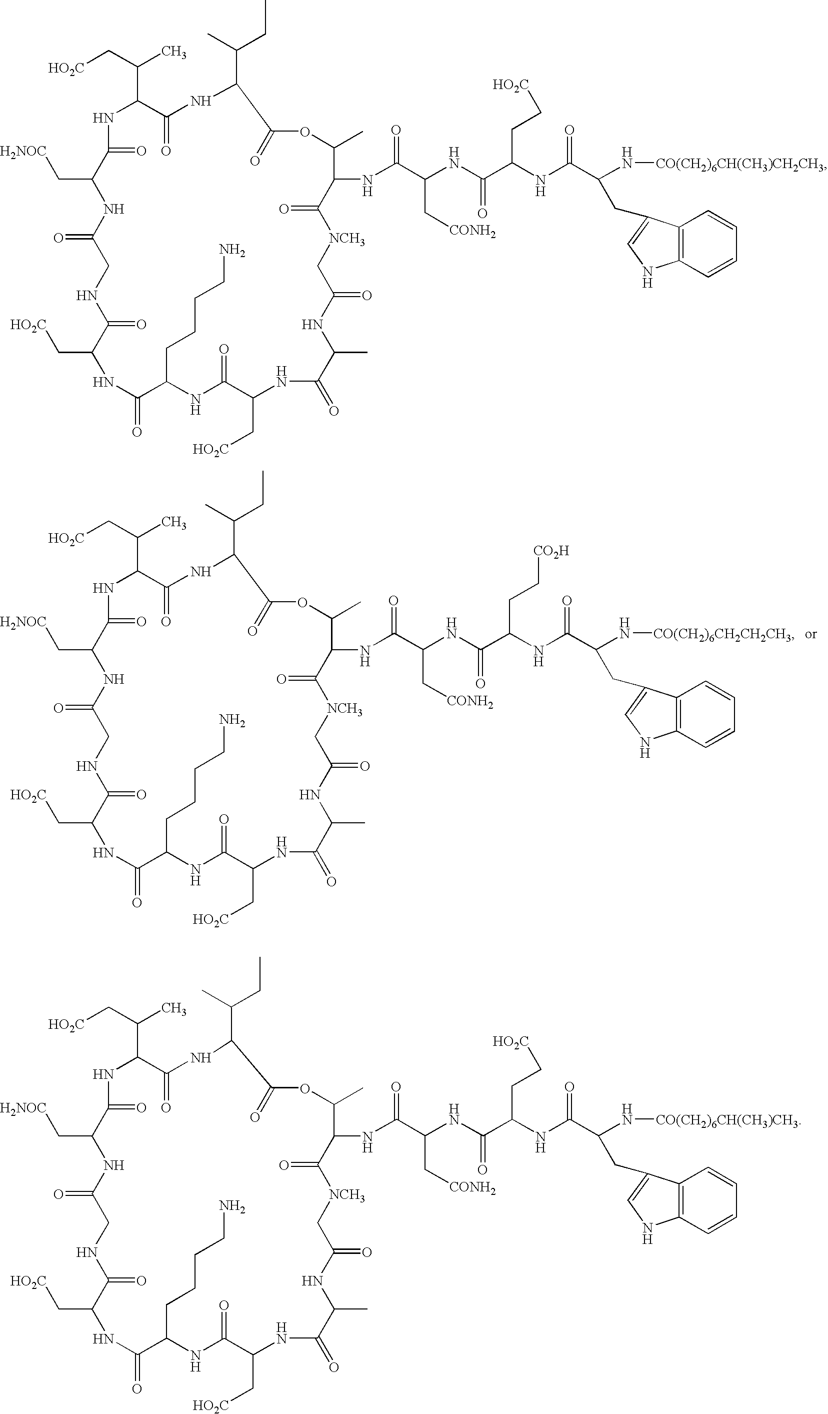

80. A composition comprising a compound of Formula F21 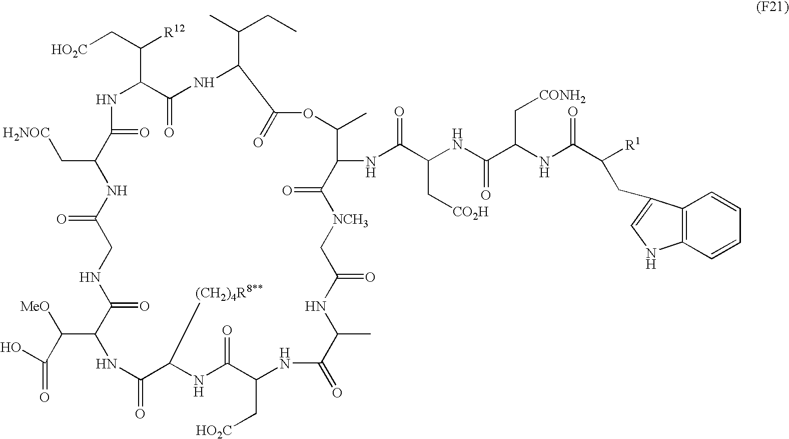
and salts thereof; wherein:
a) R¹ is 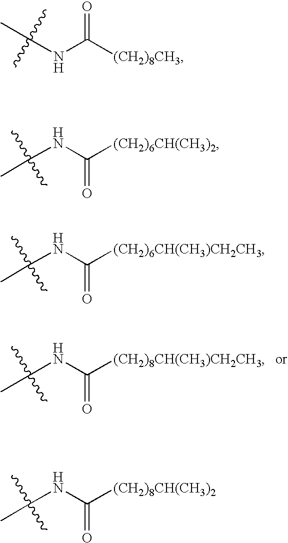
b) R¹² is H or CH₃, and
c) R⁸** is amino, monosubstituted amino, disubstituted amino, NH-amino protecting group, acylamino, ureido, guanidino, carbamoyl, sulfonamino, thioacylamino, thioureido, iminoamino, or phosphonamino.

81. The compound of claim 80 wherein R⁸** is amino, NH-amino protecting group, or carbamoyl.

82. The compound of claim 81 wherein R⁸** is amino.

83. The compound of claim 80 wherein R¹ is

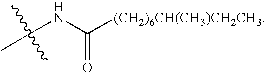

84. The compound of claim 81 wherein R¹ is

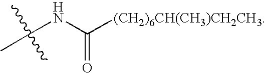

85. The compound of claim 80 wherein R¹² is methyl.

86. The compound of claim 84 wherein R¹² is methyl.

87. The compound of claim 80 selected from

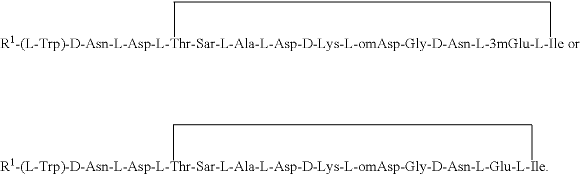

88. The compound of claim 80 selected from

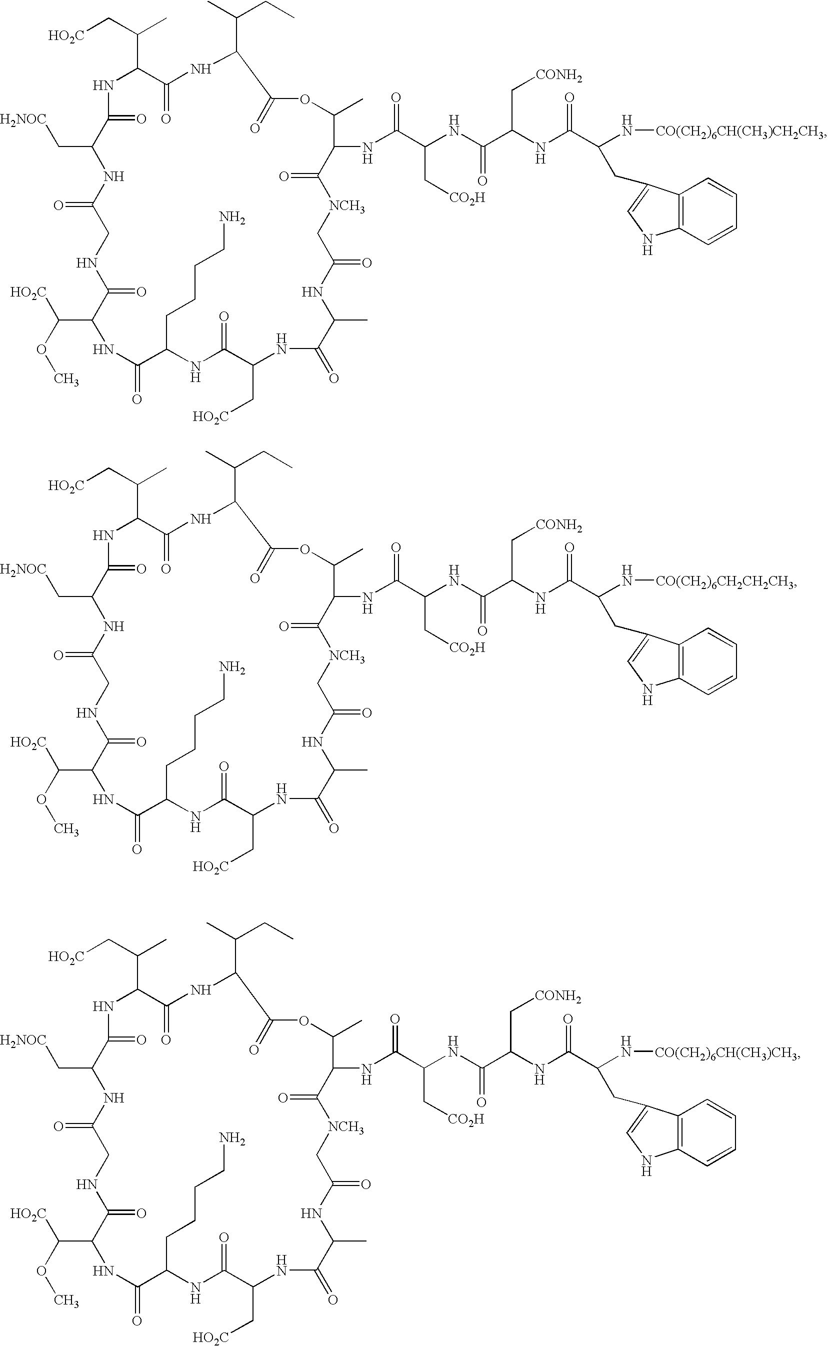

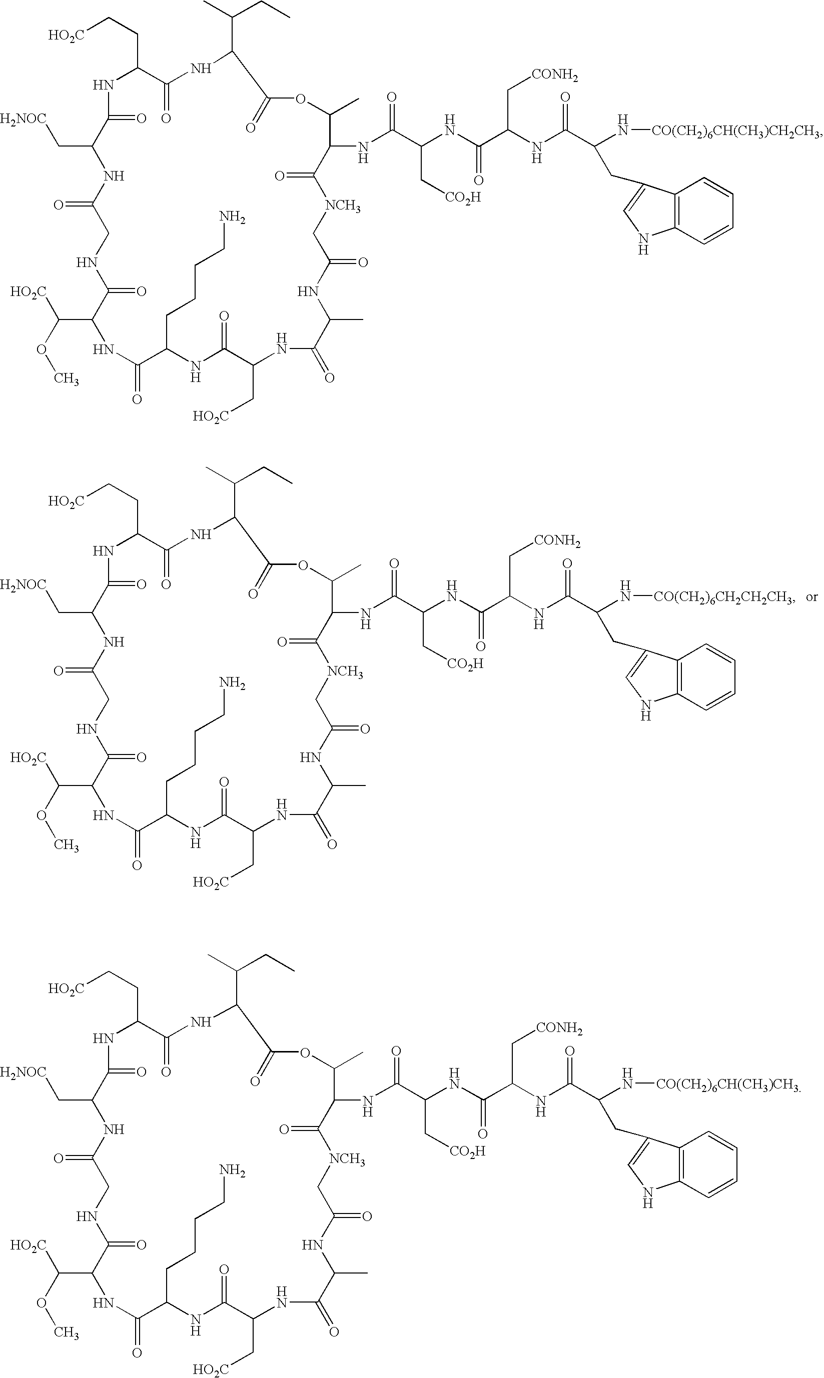

89. A pharmaceutical composition comprising a compound of claim 60 and a pharmaceutically acceptable carrier.

90. An antibacterial composition comprising a compound of claim 60 in an aqueous buffer.

91. A method of treating a bacterial infection in a subject, comprising administering a therapeutically-effective amount of the composition according to claim 60 to a subject in need thereof for a time and under conditions to ameliorate said bacterial infection.

92. Use of a composition according to claim 60 for the manufacture of a medicament for treating a bacterial infection in a subject.

93. A composition of claim 60 wherein the compound is present in an amount of about 80% to about 90% of the composition.

94. The composition according to claim 60 wherein the compound is present in about 90% of the composition.

95. The composition of claim 60 wherein the compound is present in greater than about 90% of the composition.

96. A pharmaceutical composition comprising a compound of claim 60 and a pharmaceutically acceptable carrier.

97. An antibacterial composition comprising a compound of claim 68 in an aqueous buffer.

98. A method of treating a bacterial infection in a subject, comprising administering a therapeutically-effective amount of the composition according to claim 68 to a subject in need thereof for a time and under conditions to ameliorate said bacterial infection.

99. Use of a composition according to claim 68 for the manufacture of a medicament for treating a bacterial infection in a subject.

100. A composition of claim 68 wherein the compound is present in an amount of about 80% to about 90% of the composition.

101. The composition according to claim 68 wherein the compound is present in about 90% of the composition.

102. The composition of claim 68 wherein the compound is present in greater than about 90% of the composition.


SEQUENCE LISTING (49 sequences)

SEQ ID NO: 1 - DNA, 28 bp, Artificial Sequence
  Description of Artificial Sequence Synthetic primer
gcgaagcttctggtggcgcatcacctgg

SEQ ID NO: 2 - DNA, 29 bp, Artificial Sequence
  Description of Artificial Sequence Synthetic primer
gctctagatggaagtatgtcctccatcgc

SEQ ID NO: 3 - DNA, 23 bp, Artificial Sequence
  Description of Artificial Sequence Synthetic primer
cggatcccgccggcacctgaccc

SEQ ID NO: 4 - DNA, 28 bp, Artificial Sequence
  Description of Artificial Sequence Synthetic primer
ccgaattccgcctccgagtacatcgagg

SEQ ID NO: 5 - DNA, 66 bp, Artificial Sequence
  Description of Artificial Sequence Synthetic primer
gccagcatggagccgaactgccggaacaccgcgtcccggtccacctgtgtaggctggagctgcttc

SEQ ID NO: 6 - DNA, 64 bp, Artificial Sequence
  Description of Artificial Sequence Synthetic primer
gccgactgggagtgggtcaagtggctgccgcacgtgctggatccgcatatgaatatcctcctta

SEQ ID NO: 7 - DNA, 67 bp, Artificial Sequence
  Description of Artificial Sequence Synthetic primer
ccgagacaggcaggatctcctcgactaccttcgaccggcggttcatatgtccgcctcctttggtcac

SEQ ID NO: 8 - DNA, 66 bp, Artificial Sequence
  Description of Artificial Sequence Synthetic primer
catacttcctctcactccgctgcaggagggactgctgttccacagtgtgtaggctggagctgcttc

SEQ ID NO: 9 - DNA, 76 bp, Artificial Sequence
  Description of Artificial Sequence Synthetic primer
ttgttcgaggcgccgacggtgagccgtttggagcggttgctgcgggagcgcctaggacgttgacaccatcgaatgg

SEQ ID NO: 10 - DNA, 77 bp, Artificial Sequence
  Description of Artificial Sequence Synthetic primer
acaatctcagcaccccccaccacaccaaccgccccagcgtccgaaccacgtttaaaccctcattcatcgggcgaaag

SEQ ID NO: 11 - DNA, 76 bp, Artificial Sequence; identical to SEQ ID NO: 9
  Description of Artificial Sequence Synthetic primer

SEQ ID NO: 12 - DNA, 77 bp, Artificial Sequence; identical to SEQ ID NO: 10
  Description of Artificial Sequence Synthetic primer

SEQ ID NO: 13 - DNA, 72 bp, Artificial Sequence
  Description of Artificial Sequence Synthetic primer
tcggggcgcgggtcggcggggcgcagccggggtccggcctcgcccgctagcttcttagacgtcaggtggcac

SEQ ID NO: 14 - DNA, 71 bp, Artificial Sequence
  Description of Artificial Sequence Synthetic primer
cgcgacatcttcgaacagcgcacgcccgccgccctcgccggccgcgttaaccgatacgcgagcgaacgtga

SEQ ID NO: 15 - DNA, 32 bp, Artificial Sequence
  Description of Artificial Sequence Synthetic primer
ggcggaagcttacggcacggcaaggccgtttc

SEQ ID NO: 16 - DNA, 29 bp, Artificial Sequence
  Description of Artificial Sequence Synthetic primer
ggcggcatatgaccgtgcacgactaccac

SEQ ID NO: 17 - DNA, 64 bp, Artificial Sequence
  Description of Artificial Sequence Synthetic primer
gcatccgatgcaagtgtgtcgctgtcgacggtgaccctatagtcgtgtaggctggagctgcttc

SEQ ID NO: 18 - DNA, 64 bp, Artificial Sequence
  Description of Artificial Sequence Synthetic primer
ccgaggaaaagagggaacgggacaggtcagtgaccggcgaccgtgcatatgaatatcctcctta

SEQ ID NO: 19 - DNA, 66 bp, Artificial Sequence; identical to SEQ ID NO: 8
  Description of Artificial Sequence Synthetic primer

SEQ ID NO: 20 - DNA, 64 bp, Artificial Sequence; identical to SEQ ID NO: 6
  Description of Artificial Sequence Synthetic primer

SEQ ID NO: 21 - DNA, 76 bp, Artificial Sequence
  Description of Artificial Sequence Synthetic primer
gttcgccttccccaccgtcgccggccttctcccgctcctggacgacaacctaggtgtgtaggctggagctgcttcg

SEQ ID NO: 22 - DNA, 73 bp, Artificial Sequence
  Description of Artificial Sequence Synthetic primer
tcagggcgccggtcgatcctggtcacaggtggcagggcggtgccgggtttaaaccatatgaatatcctcctta

SEQ ID NO: 23 - DNA, 72 bp, Artificial Sequence
  Description of Artificial Sequence Synthetic primer
tccgggcggggccggacgggacggacgtggtcgtccggcacggccgctagcttcttagacgtcaggtggcac

SEQ ID NO: 24 - DNA, 71 bp, Artificial Sequence
  Description of Artificial Sequence Synthetic primer
ttcgaggcgcccacgcccgccgcgctgtcccggcgcctcgacaccgttaaccgatacgcgagcgaacgtga

SEQ ID NO: 25 - DNA, 76 bp, Artificial Sequence; identical to SEQ ID NO: 9
  Description of Artificial Sequence Synthetic primer

SEQ ID NO: 26 - DNA, 78 bp, Artificial Sequence
  Description of Artificial Sequence Synthetic primer
cagctcgctgatgatatgctgacgctcaatgccgtttggcctccgactaagtttaaaccctcattcatcgggcgaaag

SEQ ID NO: 27 - DNA, 76 bp, Artificial Sequence
  Description of Artificial Sequence Synthetic primer
gtcctccgaccgcgacatccgtcgcaacgcggggcgggtgtcagggcgggctagcttcttagacgtcaggtggcac

SEQ ID NO: 28 - DNA, 71 bp, Artificial Sequence
  Description of Artificial Sequence Synthetic primer
caccgaactcgaccagctcgaagcagagtggaaggccggctgatggttaaccgatacgcgagcgaacgtga

SEQ ID NO: 29 - DNA, 78 bp, Artificial Sequence
  Description of Artificial Sequence Synthetic primer
gtgttcgaggcccgaacggtcgccgcgctggcggcccggctgcggaccgcgctagctgtgtaggctggagctgcttcg

SEQ ID NO: 30 - DNA, 72 bp, Artificial Sequence
  Description of Artificial Sequence Synthetic primer
cggcgagagcggggtcctcgtcgcctgccgcgtcggtcctgcggggtttaaaccatatgaatatcctcctta

SEQ ID NO: 31 - DNA, 73 bp, Artificial Sequence
  Description of Artificial Sequence Synthetic primer
cgagacaccgaccgtggccggtctcgccgccgcgctctccgcggccctaggtgtgtaggctggagctgcttcg

SEQ ID NO: 32 - DNA, 73 bp, Artificial Sequence
  Description of Artificial Sequence Synthetic primer
gtcccgcgaccgccgagtacctcggtcgccggaccgccggggcgcggtttaaaccatatgaatatcctcctta

SEQ ID NO: 33 - DNA, 73 bp, Artificial Sequence
  Description of Artificial Sequence Synthetic primer
cgtccgctcccgtgccaccagaggcacccgcacccccaaagccgacgctagcttcttagacgtcaggtggcac

SEQ ID NO: 34 - DNA, 74 bp, Artificial Sequence
  Description of Artificial Sequence Synthetic primer
gttcgggatgttttcgagggccgtacggtacgtgctctggcggctgtggttaaccgatacgcgagcgaacgtga

SEQ ID NO: 35 - DNA, 73 bp, Artificial Sequence
  Description of Artificial Sequence Synthetic primer
ccggtccccgaccgtcgcccgcctcgcggaggaactgggcgacgggctagctgtgtaggctggagctgcttcg

SEQ ID NO: 36 - DNA, 72 bp, Artificial Sequence
  Description of Artificial Sequence Synthetic primer
cctgcggcgcgggacgctccgcgtccgcgtccggtccggcggaccgtttaaaccatatgaatatcctcctta

SEQ ID NO: 37 - DNA, 75 bp, Artificial Sequence
  Description of Artificial Sequence Synthetic primer
aggcgctccgggcgcggaggcagcggcggggtggtgtgctgccgtccggctagcttcttagacgtcaggtggcac

SEQ ID NO: 38 - DNA, 74 bp, Artificial Sequence
  Description of Artificial Sequence Synthetic primer
ctcttcgccgcgcccacgcctgccgggctcgcgaccgtactggcggccgttaaccgatacgcgagcgaacgtga

SEQ ID NO: 39 - DNA, 73 bp, Artificial Sequence; identical to SEQ ID NO: 35
  Description of Artificial Sequence Synthetic primer

SEQ ID NO: 40 - DNA, 72 bp, Artificial Sequence
  Description of Artificial Sequence Synthetic primer
ggggggcgaccggcaggatgtcctccaaggcggtgccggtgcggcgtttaaaccatatgaatatcctcctta

SEQ ID NO: 41 - DNA, 75 bp, Artificial Sequence; identical to SEQ ID NO: 37
  Description of Artificial Sequence Synthetic primer

SEQ ID NO: 42 - DNA, 75 bp, Artificial Sequence
  Description of Artificial Sequence Synthetic primer
cgacgtgacgctggtggaagtgaaccaggtggagctcgaccgtctgcaggttaaccgatacgcgagcgaacgtga

SEQ ID NO: 43 - DNA, 73 bp, Artificial Sequence
  Description of Artificial Sequence Synthetic primer
ccggtccccgaccgtcgcccgcctcgcggaggaactgggcgacggcctaggtgtgtaggctggagctgcttcg

SEQ ID NO: 44 - DNA, 72 bp, Artificial Sequence; identical to SEQ ID NO: 36
  Description of Artificial Sequence Synthetic primer

SEQ ID NO: 45 - DNA, 72 bp, Artificial Sequence; identical to SEQ ID NO: 13
  Description of Artificial Sequence Synthetic primer

SEQ ID NO: 46 - DNA, 71 bp, Artificial Sequence; identical to SEQ ID NO: 14
  Description of Artificial Sequence Synthetic primer

SEQ ID NO: 47 - protein, 4 aa, Artificial Sequence
  Description of Artificial Sequence Synthetic peptide
  MOD_RES (2): Asp(OtBu)
  MOD_RES (3): Asn(NHTrt)
TDNW

SEQ ID NO: 48 - protein, 13 aa, Artificial Sequence
  Description of Artificial Sequence Synthetic peptide
  MOD_RES (3): 3-hydroxy-Asn
  MOD_RES (5): Sar
  MOD_RES (9): 3-methoxy-Asp
WNNTXADKDGNEI

SEQ ID NO: 49 - protein, 4 aa, Artificial Sequence
  Description of Artificial Sequence Synthetic peptide
  MOD_RES (4): Asp(OtBu)
AGTD